(19) United States
(12) Patent Application Publication
(10) Pub. No.: US 2014/0004565 A1
(43) Pub. Date: Jan. 2, 2014

(54) CELL-BASED BIOPROCESSING

(71) Applicants: Anthony Rossomando, Cambridge, MA (US); Muthiah Manoharan, Cambridge, MA (US); Gregory Hinkle, Cambridge, MA (US); Brian Bettencourt, Cambridge, MA (US); John M. Maraganora, Cambridge, MA (US); Stuart Pollard, Cambridge, MA (US); David Kocisko, Newton, MA (US); Shannon Hogan, Cambridge, MA (US)

(72) Inventors: Anthony Rossomando, Cambridge, MA (US); Muthiah Manoharan, Cambridge, MA (US); Gregory Hinkle, Cambridge, MA (US); Brian Bettencourt, Cambridge, MA (US); John M. Maraganora, Cambridge, MA (US); Stuart Pollard, Cambridge, MA (US); David Kocisko, Newton, MA (US); Shannon Hogan, Cambridge, MA (US)

(73) Assignee: Alnylam Pharmaceuticals, Inc., Cambridge, MA (US)

(21) Appl. No.: 13/380,182

(22) PCT Filed: Jul. 6, 2010
(86) PCT No.: PCT/US10/41106

§ 371 Date: Mar. 8, 2012

Related U.S. Application Data
(60) Provisional application No. 61/354,932, filed on Jun. 15, 2010.
(60) Provisional application No. 61/334,398, filed on May 13, 2010.
(60) Provisional application No. 61/319,578, filed on Mar. 31, 2010.
(60) Provisional application No. 61/319,589, filed on Mar. 31, 2010.
(60) Provisional application No. 61/293,980, filed on Jan. 11, 2010.
(60) Provisional application No. 61/267,419, filed on Dec. 7, 2009.
(60) Provisional application No. 61/244,868, filed on Sep. 22, 2009.
(60) Provisional application No. 61/223,370, filed on Jul. 6, 2009.

(51) Int. Cl. C07K 16/28

(52) U.S. Cl. CPC C07K 16/2887
USPC 435/70.3

(57) ABSTRACT

The invention provides compositions and methods for producing an immunogenic agent from a host cell. In various embodiments, the immunogenic agent is a polypeptide, an antigen, a virus particle, or a vaccine In one aspect, the invention provides for a method for producing an immunogenic agent from a host cell. The method generally comprises contacting the cell with a RNA effector molecule, a portion of which is complementary to a target gene, maintaining the cell in a large-scale bioreactor for a time sufficient to modulate expression of the target gene, wherein the modulation enhances production of the immunogenic agent from the cell, and isolating the immunogenic agent from the cell.

### RELATED APPLICATIONS

This application claims the benefit of U.S. Provisional Patent Application No. 61/319,578, filed Mar. 31, 2010, entitled CELL-BASED BIOPROCESSING, by Rossomando et al.; U.S. Provisional Patent Application No. 61/223,370, filed Jul. 6, 2009, entitled COMPOSITIONS AND METHODS FOR ENHANCING PRODUCTION OF A BIOLOGICAL PRODUCT, by Maraganore et al.; U.S. Provisional Patent Application No. 61/244,868, filed Sep. 22, 2009, entitled COMPOSITIONS AND METHODS FOR ENHANCING PRODUCTION OF A BIOLOGICAL PRODUCT, by Maraganore et al.; U.S. Provisional Patent Application No. 61/267,419, filed Dec. 7, 2009, entitled NOVEL LIPIDS AND COMPOSITIONS FOR THE DELIVERY OF THERAPEUTICS, by Manoharan et al., filed; U.S. Provisional Patent Application No. 61/334,398, filed May 13, 2010, entitled CHARGED LIPIDS AND COMPOSITIONS FOR NUCLEIC ACID DELIVERY, by Manoharan et al.; U.S. Provisional Patent Application No. 61/293,980, filed Jan. 11, 2010, entitled COMPOSITIONS AND METHODS FOR ENHANCING PRODUCTION OF A BIOLOGICAL PRODUCT, by Rossomando et al.; U.S. Provisional Patent Application No. 61/319,589, filed Mar. 31, 2010, entitled CELL-BASED BIOPROCESSING, by Rossomando et al.; and U.S. Provisional Patent Application No. 61/354,932, filed Jun. 15, 2010, entitled CHINESE HAMSTER OVARY (CHO) CELL TRANSCRIPTOME, CORRESPONDING SIRNAS AND USES THEREOF, by Rossomando et al.; each of which is incorporated fully herein by reference.

### REFERENCES TO TABLES AND SEQUENCES

The specification includes a Sequence Listing as part of the originally filed subject matter. The sequence listing for SEQ ID NOs 1 to 3,290,939 is provided herein in an electronic format on 4 compact discs (CD-R), labeled “CRF,” “COPY 1,” “COPY 2,” and “COPY 3,” as file name “51058077.TXT,” and is incorporated herein by reference in their entirety in to the present specification.

The instant application contains a “lengthy” Sequence Listing which has been submitted via CD-R in lieu of a printed paper copy, and is hereby incorporated by reference in its entirety. Said CD-R, recorded on Jul. 1, 2010, are labeled CRF, “Copy 1,” “Copy 2” and “Copy 3”, respectively, and each contains only one identical 774,635 KB file (51058077.TXT).

### FIELD OF THE INVENTION

The invention relates generally to the field of bioprocessing and more particularly to methods for producing an immunogenic agent in a host cell by contacting the cell with a RNA effector molecule capable of modulating expression of a target gene, wherein the modulation enhances production of the immunogenic agent. The invention also relates generally to transcriptomes, organized transcriptomes, and systems and methods using the transcriptomes for designing targeted modulation of immunogenic agent production in cells. The invention further relates to engineering cells and cell lines for more effective and efficient production of immunogenic agents. The invention also relates to molecules, compositions, cells, and kits useful for carrying out the methods and immunogenic agent produced by the methods.

### BACKGROUND

Cell culture techniques are used to manufacture a wide range of biological products, including biopharmaceuticals, biofuels, metabolites, vitamins, nutraceuticals, immunogenic agents and vaccines. A number of strategies have been developed to enhance productivity, yield, efficiency, and other aspects of cell culture bioprocesses in order to facilitate industrial scale production and meet applicable standards for product quality and consistency. Traditional strategies for optimizing cell culture bioprocesses involve adjusting physical and biochemical parameters, such as culture media (e.g., pH, nutrients) and conditions (e.g., temperature, duration), and selecting host cells having desirable phenotypes. Genetic approaches have also been developed for optimizing cell culture bioprocesses by introducing recombinant DNA into host cells, where the DNA encodes an exogenous protein that influences the production of an immunogenic agent, or regulates expression of an endogenous protein that influences production of the immunological agent. Such methods require costly and time-consuming laboratory manipulations, however, and can be incompatible with certain genes, proteins, host cells, and biological products including immunogenic agents. Accordingly, there is a need in the art for new genetic approaches for optimizing cell culture bioprocesses involving a wide range of host cells and biological products, such as immunogenic agents.

More recently, host cells for biological production have been modified to incorporate into their genome genes that express shRNAs for the silencing of genes that influence production of the biological product. In these cases, product yield has proven difficult to regulate, however, because of uncontrolled, unintended, expression of the shRNAs which compromises host cell viability. The process of incorporating shRNAs also requires cell engineering, which is time-consuming. Furthermore, uncontrolled expression ultimately leads to phenotypic changes and overtime the host cells carrying the genes for expressed shRNA lose their ability to produce biological product at any significant yield.

For example, Chinese hamster (Cricetulus griseus) ovary cells (CHO cells) have been used widely in various bioprocesses, yet relatively little is known about gene expression s in these cells; thus, targeted and intelligent modulation of bioprocesses in these cells cannot be done or designed readily. Accordingly, there is a need in the art for new genetic approaches for optimizing cell culture bioprocesses involving a wide range of host cells, including CHO cells, and immunogenic agents produced in these cells.

### SUMMARY

The invention is based at least in part on the surprising discovery that RNA effector molecules can be applied at low concentrations to cells in culture to effect potent, durable modulation of gene expression, such that the quality and quantity of an immunogenic agent produced by a host cell can be improved without the need for extensive cell line engineering. As such, in a first aspect, the invention provides compositions and methods for producing an immunogenic agent from a host cell. In various embodiments, the immunogenic agent is a polypeptide, a viral product, a virus particle, or a vaccine.

In one aspect, the invention provides for a method for producing an immunogenic agent from a host cell. The method generally comprises contacting the cell with a RNA effector molecule, a portion of which is complementary to a target gene, maintaining the cell in a large-scale bioreactor for a time sufficient to modulate expression of the target gene, wherein the modulation enhances production of the immunogenic agent from the cell, and isolating the immunogenic agent from the cell.

In one embodiment, the RNA effector molecule transiently modulates expression of the target gene. In another embodiment, the RNA effector molecule transiently inhibits expression of the target gene. In one embodiment, the RNA effector molecule can activate the target gene. In another embodiment, the RNA effector can inhibit the target gene.

In further embodiments, the host cell is an animal cell, a plant cell, an insect cell, or a fungal cell. In one embodiment, the animal cell is a mammalian cell. In a further embodiment, the mammalian cell is a human cell, a rodent cell, a canine cell, or a non-human primate cell. In a particular embodiment, the host cell is a cell derived from a CHO cell. In another embodiment, a host cell contains a transgene that encodes an immunogenic agent.

In one embodiment, the cell is contacted with a plurality of different RNA effector molecules. The plurality of RNA effector molecules can be used to modulate expression of a single target gene or multiple target genes.

In another embodiment, the composition is formulated for administration to cells according to a dosage regimen described herein, e.g., at a frequency of 6 hr, 12 hr, 24 hr, 36 hr, 48 hr, 72 hr, 84 hr, 96 hr, 108 hr, or more. In another embodiment, the administration of the composition can be maintained during one or more cell growth phases, e.g., lag phase, early log phase, mid-log phase, late-log phase, stationary phase, or death phase. In some of the embodiments, contacting a host cell with a RNA effector molecule (e.g., a dsRNA) occurs prior to, during or after the viral infection or vector inoculation to inhibit cellular and/or anti-viral processes that compromise the yield and quality of the immunogenic agent harvest.

In another embodiment, a composition containing two or more RNA effector molecules directed against separate target genes is used to enhance production of a immunogenic agent in cell culture by modulating expression of a first target gene and at least a second target gene in the cultured cells. In another embodiment, a composition containing two or more RNA effector molecules directed against the same target gene is used to enhance production of an immunogenic agent in cell culture by modulating expression of the target gene in cultured cells.

In another embodiment, a first RNA effector molecule is administered to a cultured cell, and then a second RNA effector molecule is administered to the cell (or vice versa). In a further embodiment, the first and second RNA effector molecules are administered to a cultured cell substantially simultaneously.

In one embodiment, the RNA effector molecule is added to the cell culture medium used to maintain the cells under conditions that permit production of an immunogenic agent. The RNA effector molecule can be added at different times or simultaneously. In one embodiment, one or more of the different RNA effector molecules are added by continuous infusion into the cell culture medium, for example, to maintain a continuous average percent inhibition or RNA effector molecule concentration. In another embodiment, one or more of the different RNA effector molecules are added by continuous infusion into the cell culture medium, for example, to maintain a minimum average percent inhibition or RNA effector molecule concentration. In one embodiment, the continuous infusion is administered at a rate to achieve a desired average percent inhibition for at least one target gene. In one embodiment, the continuous infusion is performed for a distinct period of time (which can be repeated), e.g., for 1 hr, 2 hr, 3 hr, 4 hr, 8 hr, 16 hr, 18 hr, 24 hr, 48 hr, 72 hr, or longer. When applying a plurality of different RNA effector molecules, each of the different RNA effector molecules can be added at the same frequency or different frequencies. Each of the different RNA effector molecules is added at the same concentration or at different concentrations. In some embodiments, the last contact of cells with a RNA effector molecule is at least 24 hr, 48 hr, 72 hr, 120 hr, or later, before isolation of the immunogenic agent or harvesting the supernatant.

Generally, the RNA effector molecule is added at a given concentration of less than or equal to 200 nM (e.g., 100 nM, 80 nM, 50 nM, 20 nM, 10 nM, 1 nM, or less). As described herein, low concentrations of RNA effector molecules can be used in large scale bioprocessing to efficiently modulate target genes. There are significant economic and commercial advantages (e.g., lower costs and easier removal) of using low concentrations of RNA effector molecules. Thus, in one embodiment, cells are contacted with a RNA effector molecule at a concentration of 100 nM or less, 50 nM or less, 20 nM or less, 10 nM or less, 5 nM or less, or 1 nM or less. In a particular embodiment, the one or more RNA effector molecules is administered into the cell culture medium at a final concentration of 1 nM at least once (e.g., at least two times, at least three times, at least four times, or more) during the growth phase and/or production phase.

In still another embodiment, the RNA effector molecule is added at a given starting concentration of each of the different RNA effector molecules (e.g., at 1 nM each), and further supplemented with continuous infusion of the RNA effector molecule.

In one embodiment, the RNA effector composition comprises a reagent that facilitates RNA effector molecule uptake, for example, an emulsion, a cationic lipid, a non-cationic lipid, a charged lipid, a liposome, an anionic lipid, a penetration enhancer, a transfection reagent or a modification to the RNA effector molecule for attachment, e.g., a ligand, a targeting moiety, a peptide, a lipophilic group, etc.

The RNA effector molecule to be contacted with the cell can be incorporated into a formulation that facilitates uptake and delivery into the cell. The one or more of the different RNA effector molecules can be added by contacting the cells with the RNA effector molecule and a reagent that facilitates RNA effector molecule uptake, for example, an emulsion, a cationic lipid, a non-cationic lipid, a charged lipid, a liposome, an anionic lipid, a penetration enhancer, a transfection reagent or a modification to the RNA effector molecule for attachment, e.g., a ligand, a targeting moiety, a peptide, a lipophilic group, etc.

In certain embodiments, a lipid formulation is used in a RNA effector molecule composition as a reagent that facilitates RNA effector molecule uptake. In certain embodiments, the lipid formulation can be a LNP formulation, a LNP01 formulation, a XTC-SNALP formulation, or a SNALP formulation as described herein. In related embodiments, the XTC-SNALP formulation is as follows: using 2,2-Dilinoleyl-4-dimethylaminoethyl-[1,3]-dioxolane (XTC) with XTC/DPPC/Cholesterol/PEG-cDMA in a ratio of 57.1/7.1/34.4/1.4 and a lipid:siRNA ratio of about 7. In still other related embodiments, the RNA effector molecule is a dsRNA and is formulated in a XTC-SNALP formulation as follows: using 2,2-Dilinoleyl-4-dimethylaminoethyl-[1,3]-dioxolane (XTC) with a XTC/DPPC/Cholesterol/PEG-cDMA in a ratio of 57.1/7.1/34.4/1.4 and a lipid:siRNA ratio of about 7. Alternatively, a RNA effector molecule such as those described herein can be formulated in a LNP09 formulation as follows: using XTC/DSPC/Chol/PEG2000-C14 in a ratio of 50/10/38.5/1.5 mol % and a lipid:siRNA ratio of about 11:1. In some embodiments, the RNA effector molecule is formulated in a LNP11 formulation as follows: using MC3/DSPC/Chol/PEG2000-C14 in a ratio of 50/10/38.5/1.5 mol % and a lipid:siRNA ratio of about 11:1. In still another embodiment, the RNA effector molecule is formulated in a LNP09 formulation or a LNP 11 formulation and reduces the target gene mRNA levels by about 85 to 90% at a dose of 0.3 mg/kg, relative to a PBS control group. In yet another embodiment, the RNA effector molecule is formulated in a LNP09 formulation or a LNP11 formulation and reduces the target gene mRNA levels by about 50% at a dose of 0.1 mg/kg, relative to a PBS control group. In yet another embodiment, the RNA effector molecule is formulated in a LNP09 formulation or a LNP11 formulation and reduces the target gene protein levels in a dose-dependent manner relative to a PBS control group as measured by a western blot. In yet another embodiment, the RNA effector molecule is formulated in a SNALP formulation as follows: using DlinDMA with a DLinDMA/DPPC/Cholesterol/PEG2000-cDMA in a ratio of 57.1/7.1/34.4/1.4 and a lipid:siRNA ratio of about 7.

In some embodiments, the lipid formulation comprises a lipid having the following formula:

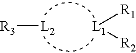

where R₁ and R₂ are each independently for each occurrence optionally substituted C₁₀-C₃₀ alkyl, optionally substituted C₁₀-C₃₀ alkoxy, optionally substituted C₁₀-C₃₀ alkenyl, optionally substituted C₁₀-C₃₀ alkenyloxy, optionally substituted C₁₀-C₃₀ alkynyl, optionally substituted C₁₀-C₃₀ alkynyloxy, or optionally substituted C₁₀-C₃₀ acyl;

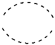

represents a connection between L₂ and L₁ which is:

(1) a single bond between one atom of L₂ and one atom of L₁, wherein
L₁ is C(Rₓ), O, S or N(Q);
L₂ is —CR₅R₆—, —O—, —S—, —N(Q)-, ═C(R₅)—, —C(O)N(Q)-, —C(O)O—, —N(Q)C(O)—, —OC(O)—, or —C(O)—;

(2) a double bond between one atom of L₂ and one atom of L₁; wherein

L₁ is C;
L₂ is —CR₅═, —N(Q)═, —N—, —O—N═, —N(Q)-N═, or —C(O)N(Q)-N═;

(3) a single bond between a first atom of L₂ and a first atom of L₁, and a single bond between a second atom of L₂ and the first atom of L₁, wherein
L₁ is C;
L₂ has the formula

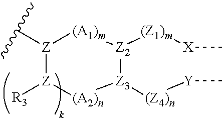

wherein
X is the first atom of L₂, Y is the second atom of L₂, - - - - - represents a single bond to the first atom of L₁, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
Z₁ and Z₄ are each, independently, —O—, —S—, —CH₂—, —CHR⁵—, or —CR⁵R⁵—;
Z₂ is CH or N;
Z₃ is CH or N;
or Z₂ and Z₃, taken together, are a single C atom;

A₁ and A₂ are each, independently, —O—, —S—, —CH₂—, —CHR⁵—, or —CR⁵R⁵—;
each Z is N, C(R₅), or C(R₃);
k is 0, 1, or 2;
each m, independently, is 0 to 5;
each n, independently, is 0 to 5;

where m and n taken together result in a 3, 4, 5, 6, 7 or 8 member ring;

(4) a single bond between a first atom of L₂ and a first atom of L₁, and a single bond between the first atom of L₂ and a second atom of L₁, wherein

(A) L₁ has the formula:

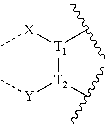

wherein
X is the first atom of L₁, Y is the second atom of L₁, - - - - - -represents a single bond to the first atom of L₂, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
T₁ is CH or N;
T₂ is CH or N;
or T₁ and T₂ taken together are C═C;
L₂ is CR₅; or

(B) L₁ has the formula:

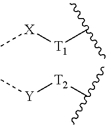

wherein

X is the first atom of L₁, Y is the second atom of L₁, - - - - -represents a single bond to the first atom of L₂, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
T₁ is —CR₅R₅—, —N(Q)-, —O—, or —S—;
T₂ is —CR₅R₅—, —N(Q)-, —O—, or —S—;
L₂ is CR₅ or N;

R₃ has the formula:

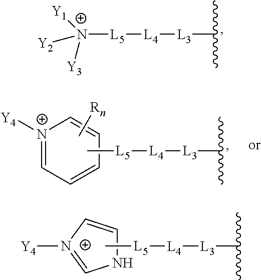

wherein

each of Y₁, Y₂, Y₃, and Y₄, independently, is alkyl, cycloalkyl, aryl, aralkyl, or alkynyl; or

any two of Y₁, Y₂, and Y₃ are taken together with the N atom to which they are attached to form a 3- to 8-member heterocycle; or

Y₁, Y₂, and Y₃ are all be taken together with the N atom to which they are attached to form a bicyclic 5- to 12-member heterocycle;

each Rₙ, independently, is H, halo, cyano, hydroxy, amino, alkyl, alkoxy, cycloalkyl, aryl, heteroaryl, or heterocyclyl;

L₃ is a bond, —N(Q)-, —O—, —S—, —(CR₅R₆)ₐ—, —C(O)—, or a combination of any two of these;

L₄ is a bond, —N(Q)-, —O—, —S—, —(CR₅R₆)ₐ—, —C(O)—, or a combination of any two of these;

L₅ is a bond, —N(Q)-, —O—, —S—, —(CR₅R₆)ₐ—, —C(O)—, or a combination of any two of these;

each occurrence of R₅ and R₆ is, independently, H, halo, cyano, hydroxy, amino, alkyl, alkoxy, cycloalkyl, aryl, heteroaryl, or heterocyclyl; or two R₅ groups on adjacent carbon atoms are taken together to form a double bond between their respective carbon atoms; or two R₅ groups on adjacent carbon atoms and two R₆ groups on the same adjacent carbon atoms are taken together to form a triple bond between their respective carbon atoms;

each a, independently, is 0, 1, 2, or 3;

wherein

an R₅ or R₆ substituent from any of L₃, L₄, or L₅ is optionally taken with an R₅ or R₆ substituent from any of L₃, L₄, or L₅ to form a 3- to 8-member cycloalkyl, heterocyclyl, aryl, or heteroaryl group; and

any one of Y₁, Y₂, or Y₃, is optionally taken together with an R₅ or R₆ group from any of L₃, L₄, and L₅, and atoms to which they are attached, to form a 3- to 8-member heterocyclyl group;

each Q, independently, is H, alkyl, acyl, cycloalkyl, alkenyl, alkynyl, aryl, heteroaryl or heterocyclyl; and

each Q₂, independently, is O, S, N(Q)(Q), alkyl or alkoxy.

In a particular embodiment, the formulation comprises a lipid containing a quaternary amine, such as those described herein (for example, Lipid H, Lipid K, Lipid L, Lipid M, Lipid P, and Lipid R). Thus, in some embodiments, the RNA effector molecule composition comprises a reagent that facilitates RNA effector molecule uptake which comprises “Lipid H”, “Lipid K”, “Lipid L”, “Lipid M”, “Lipid P”, or “Lipid R”, whose formulae are indicated as follows:

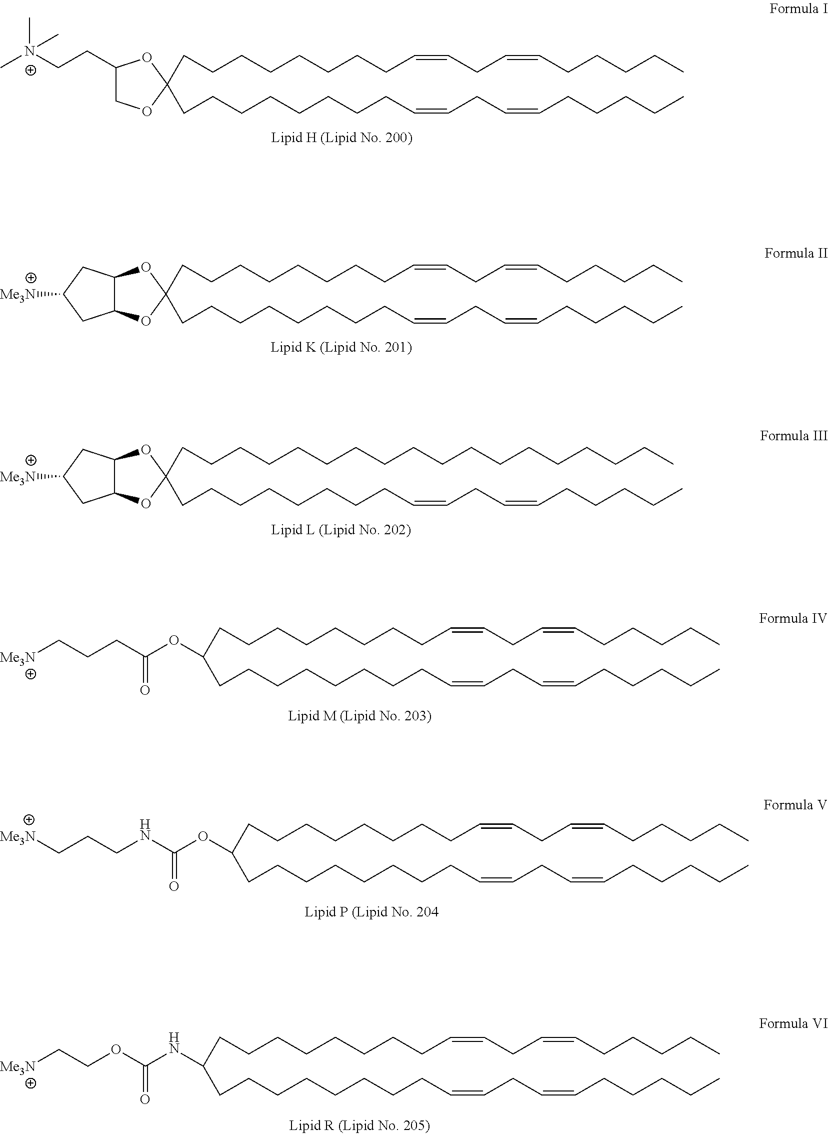

In embodiments in which the RNA effector molecule composition is formulated with a delivery facilitating agent, the composition can be in solution (e.g., a sterile solution, for example, packaged in a unit dosage form), or as a sterile lyophilized composition (pre-dosed, for example, in units for use in 1 L of cell culture media).

In another embodiment, the RNA effector molecule composition further comprises a growth medium (e.g., chemically defined media such as Biowhittaker® POWERCHO® medium (Lonza), HYCLONE PF CHO™ medium (Thermo Scientific), GIBCO® CD DG44 MEDIUM (Invitrogen, Carlsbad, Calif.), Medium M199 (Sigma-Aldrich), OPTIPRO™ SFM medium (Gibco), etc.). The RNA effector can be present in a concentration such that, when reconstituted in a medium, provides the desired concentration.

In still another embodiment, the RNA effector molecule composition further comprises an agent selected from the group consisting of essential amino acids (e.g., glutamine), 2-mercapto-ethanol, bovine serum albumin (BSA), lipid concentrate, cholesterol, catalase, insulin, human transferrin, superoxide dismutase, biotin, DL α-tocopherol acetate, DL α-tocopherol, vitamins (e.g., Vitamin A), choline chloride, D-calcium pantothenate, folic acid, Nicotinamide, pyridoxal hydrochloride, riboflavin, thiamine hydrochloride, i-Inositol, corticosterone, D-galactose, ethanolamine HCl, glutathione (reduced), L-carnitine HCl, linoleic acid, linolenic acid, progesterone, putrescine 2HCl, sodium selenite, T3 (triodo-I-thyronine), growth factors (e.g., EGF), iron, L-glutamine, L-alanyl-L-glutamine, sodium hypoxanthine, aminopterin and thymidine, arachidonic acid, ethyl alcohol 100%, myristic acid, oleic acid, palmitic acid, palmitoleic acid, PLURONIC F68® (Invitrogen), stearic acid 10, TWEEN 80® nonionic surfactant (Invitrogen), sodium pyruvate, and glucose.

In various embodiments, the RNA effector molecule can comprise siRNA, miRNA, dsRNA, saRNA, shRNA, piRNA, tkRNAi, eiRNA, pdRNA, a gapmer, an antagomir, or a ribozyme. In one embodiment the RNA effector molecule is not shRNA. In one embodiment the RNA effector molecule is a dsRNA.

In some embodiments, the RNA effector molecule is selected from a group of siRNAs, wherein the RNA effector molecule comprises sense strand and an antisense strand comprising at least 16 contiguous nucleotides (e.g., at least 17, at least 18, at least 19 nucleotides, etc.). In one embodiment, the antisense strand comprises at least 16 contiguous nucleotides. In one embodiment, the antisense strand comprises at least 17 contiguous nucleotides. In one embodiment, the antisense strand comprises at least 18 contiguous nucleotides. In one embodiment, the antisense strand comprises at least 19 contiguous nucleotides. In one embodiment, the antisense strand further comprises at least one deoxyribonucleotide. In one embodiment, the antisense strand further comprises at least two deoxyribonucleotides. In one embodiment, the antisense strand further comprises two deoxythymidine residues.

In some embodiments, the RNA effector molecule comprises an antisense strand of a double-stranded oligonucleotide in which the antisense strand comprises at least 16 contiguous nucleotides (e.g., 17, nucleotides, 18 nucleotides, or 19 nucleotides). In one embodiment, the antisense strand comprises at least 16 contiguous nucleotides. In one embodiment, the antisense strand comprises at least 17 contiguous nucleotides. In one embodiment, the antisense strand comprises at least 18 contiguous nucleotides. In one embodiment, the antisense strand comprises at least 19 contiguous nucleotides. In one embodiment, the antisense strand further comprises at least one deoxyribonucleotide. In one embodiment, the antisense strand further comprises at least two deoxyribonucleotides. In one embodiment, the antisense strand further comprises two deoxythymidine residues.

In some embodiments, the maintaining step further comprises monitoring at least one measurable parameter selected from the group consisting of cell density, medium pH, oxygen levels, glucose levels, lactic acid levels, temperature, and protein production.

In some embodiments, at least one measurable parameter can be monitored during production of an immunogenic agent, including any one of cell density, medium pH, oxygen levels, glucose levels, lactic acid levels, temperature, and protein production.

In further embodiments, the method further comprises administering to the host cell a second agent. The second agent can be a growth factor; an apoptosis inhibitor; a kinase inhibitor; a phosphatase inhibitor; a protease inhibitor; an inhibitor of pathogens (e.g., where a virus is the immunogenic agent, an agent that inhibits growth and/or propagation of other viruses or fungal or bacterial pathogens); or a histone demethylating agent. Where the virus being propagated is influenza, the second agent can be a protease that cleaves influenza hemagglutinin, such as pronase, thermolysin, subtilisin A, or a recombinant protease.

In another embodiment, a composition containing a RNA effector molecule described herein, e.g., a dsRNA directed against a host cell target gene, is administered to a cultured cell with a non-RNA agent useful for enhancing the production of an immunogenic agent by the cell. The non-RNA agent can be selected from the group consisting of: an antibiotic, an antimycotic, an antimetabolite (e.g., methotrexate), an antibody; a growth factor (e.g., insulin); an apoptosis inhibitor; a kinase inhibitor, such as a MAP kinase inhibitor, a CDK inhibitor, and/or a K252a; a phosphatase inhibitor, such as sodium vanadate and okadaic acid; a protease inhibitor; and a histone demethylating agent, such as 5-azacytidine.

In some embodiments, the immunogenic agent is a polypeptide and the target gene encodes a protein that affects post-translational modification in the host cell. In various embodiments, the post-translational modification can be protein glycosylation, protein deamidation, protein disulfide bond formation, methionine oxidation, protein pyroglutamation, protein folding, or protein secretion.

In additional embodiments, the target gene encodes a protein that affects a physiological process of the host cell. In various embodiments, the physiological process is apoptosis, cell cycle progression, cellular immune response, carbon metabolism or transport, lactate formation, RNAi uptake and/or efficacy, or actin dynamics.

In further embodiments, the target gene encodes a pro-oxidant enzyme, or a protein that affects cellular pH.

In another aspect, the invention provides a cultured eukaryotic cell containing at least one RNA effector molecule provided herein. The cell is a mammalian cell, such as a rodent cell, a canine cell, a non-human primate cell, or a human cell.

In another aspect, the invention provides a composition for enhancing production of an immunogenic agent in cell culture by modulating the expression of a target gene in a host cell. The composition typically includes one or more RNA effector molecules described herein and a suitable carrier or delivery vehicle, e.g., an acceptable carrier and/or a reagent that facilitates RNA effector molecule uptake. The RNA effector molecule composition can be formulated as suspension in aqueous, non-aqueous, or mixed media and can be formulated in a lipid or non-lipid formulation. The RNA effector molecule composition can be provided in a sterile solution or lyophilized (e.g., provided in discrete units by concentration and/or volume).

In another embodiment, a composition containing a RNA effector molecule described herein, e.g., a dsRNA directed against a host cell target gene, is administered to a cultured cell with a non-RNA agent useful for enhancing the production of an immunogenic agent by the cell.

In one embodiment, a vector is provided for modulating the expression of a target gene in a cultured cell, where the target gene encodes a protein that affects production of an immunogenic agent by the cell. In one embodiment, the vector includes at least one regulatory sequence operably linked to a nucleotide sequence that encodes at least one strand of a RNA effector molecule. In one embodiment, the RNA effector molecule is not encoded by a vector.

In another embodiment, the invention provides a cell containing a vector for inhibiting the expression of a target gene in a cell. The vector includes a regulatory sequence operably linked to a polynucleotide encoding at least one strand of a RNA effector molecule.

Still another aspect of the invention encompasses kits comprising RNA effector molecules described herein. In one embodiment, the kits comprise a RNA effector molecule that modulates expression of a target gene encoding a protein that affects production of the immunogenic agent. In another embodiment, the kits further comprise a modified cell line which expresses a RNA effector molecule which modulates expression of a protein that affects production of the immunogenic agent. The kits can also comprise instructions for carrying out methods provided herein.

In one embodiment, the kit further comprises suitable culture media for growing host cells and/or constructs (e.g., plasmid, viral, etc.) for introducing a nucleic acid sequence encoding a RNA effector molecule into host cells. In still another embodiment, the kits can further comprise reagents for detecting and/or purifying the immunogenic agent. Non-limiting examples of suitable reagents include PCR primers, polyclonal antibodies, monoclonal antibodies, affinity chromatography media, and the like.

In one embodiment, a kit comprises a RNA effector molecule that modulates expression of a target gene to inhibit expression of a latent, adventitious, or endogenous virus and thus affect production of the desired immunogenic agent. In another embodiment, a kit comprises a host cell that expresses a RNA effector molecule that modulates expression of latent, adventitious, or endogenous virus that affects production of the desired immunogenic agent. Such kits can also comprise instructions for carrying out methods provided herein. The kits can also include at least one reagent that facilitates RNA effector molecule-uptake, comprising a charged lipid, an emulsion, a liposome, a cationic or non-cationic lipid, an anionic lipid, a transfection reagent or a penetration enhancer. In a particular embodiment, the reagent that facilitates RNA effector molecule-uptake comprises a charged lipid.

Some embodiments of the present invention relate to initiating RNA interference in a host cell, during or after microbial inoculation or vector transduction, to inhibit expression of endogenous, latent or adventitious virus that can compromise the yield and/or quality of the harvested immunogenic agent. For example, an embodiment administers a siRNA, or, e.g., a shRNA in naked, conjugated or formulated form (e.g., lipid nanoparticle), that targets an endogenous, latent or adventitious virus pathway (e.g., ev loci of endogenous avian leukosis virus (ALV-E) in avian cells; endogenous type C retrovirus-like particle genomes in CHO cells; or the rep gene of porcine circovirus type 1 (PCV-1) in Vero cells), and thereby increases quality and/or yield of the desired immunogenic agent.

In some embodiments of the invention, simple naked (i.e., unconjugated) RNA effector molecules, or conjugated (e.g., directly conjugated to cholesterol or other targeting ligands) RNA effector molecules can be used. In another embodiment, plasmid- or viral vector-encoded RNA effector molecules for shRNA can be used.

In some embodiments of the invention, LNP or alternate polymer formulations are used. In some embodiments, the formulation includes an agent that facilitates RNA effector molecule-uptake, e.g., a charged lipid, an emulsion, a liposome, a cationic or non-cationic lipid, an anionic lipid, a transfection reagent or a penetration enhancer. In a particular embodiment, the reagent that facilitates RNA effector molecule-uptake comprises a charged lipid. In addition, the formulations can be co-formulated or incorporated into the infective seed or vectors themselves to facilitate delivery or stabilize RNAi materials to the relevant cell where the agent/vector can produce the desired immunogenic agent.

In particular embodiments, the target gene is associated with endogenous, adventitious or latent herpesviruses, polyomaviruses, hepadnaviruses, papillomaviruses, adenoviruses, poxviruses, bornaviruses, retroviruses, arenaviruses, orthomyxoviruses, paramyxoviruses, reoviruses, picornaviruses, flaviviruses, rabdoviruses, hantaviruses, circoviruses, or vesiviruses.

Particular endogenous and latent viruses that can be targeted by the methods of the present invention include Minute Virus of Mice (MVM), Murine leukemia/sarcoma (MLV), Circoviruses including porcine circovirus (PCV-1, PCV-2), Human herpesvirus 8 (HHV-8), arenavirus Lymphocytic choriomeningitis virus (LCMV), Lactate dehydrogenase virus (LDH or LDV), human species C adenoviruses, avian adeno-associated virus (AAV), primate endogenous retrovirus family K (ERV-K), and human endogenous retrovirus K (HERV-K).

Further regarding ERVs, in embodiments of the present invention the target genes of ERVs can be those of primate/human Class I Gamma ERVs pt01-Chr10r-17119458, pt01-Chr5-53871501, BaEV, GaLV, HERV-T, ERV-3, HERV-E, HERV-ADP, HERV-I, MER4like, HERV-FRD, HERV-W, HERVH-RTVLH2, HERVH-RGH2, HERV-Hconsensus, HERV-Fcl; primate/human Epsilon ERV hg15-chr3-152465283; primate/human Intermediate (epsilon-like) HERVL66; primate/human Class III Spuma-like ERVs HSRV, HFV, HERV-S, HERV-L, HERVL40, HERVL74; primate/human Delta ERVs HTLV-1, HTLV-2; primate/human Lenti ERVs HIV-1, HIV-2; primate/human Class II, Beta ERV MPMV, MMTV, HML1, HML2, HML3, HML4, HML7, HML8, HML5, HML10, HML6, or HML9.

In other embodiments of the present invention, the ERV is selected from rodent Class II, Beta ERV MMTV; rodent Class I Gamma ERV MLV; feline Class I Gamma ERV FLV; ungulate Class I Gamma ERV PERV; ungulate Delta ERV BLV; ungulate lentivirus Visna, EIAV; ungulate Class II, Beta ERV JSRV; avian Class III, Spuma-like ERVs gg01-chr7-7163462; gg01-chrU-52190725, gg01-Chr4-48130894; avian Alpha ERVs ALV, gg01-chr1-15168845; avian Intermediate Beta-like ERVs gg01-chr4-77338201; gg01-ChrU-163504869, gg01-chr7-5733782; Reptilian Intermediate Beta-like ERV Python-molurus; Fish Epsilon ERV WDSV; fish Intermediate (epsilon-like) ERV SnRV; Amphibian Epsilon ERV Xen1; Insect Errantivirus ERV Gypsy.

Other embodiments of the present invention target adventitious viruses of animal-origin, such as vesivirus, circovirus, hantaan virus, Marburg virus, SV40, SV20, Semliki Forest virus (SFV), simian virus 5 (sv5), lymphocytic choriomeningitis virus, feline sarcoma virus, porcine parvovirus, adenoassociated viruses (AAV), mouse hepatitis virus (MHV), murine leukemia virus (MuLV), pneumonia virus of mice (PVM), Theiler's encephalomyelitis virus (THEMV), murine minute virus (MMV or MVM), mouse adenovirus (MAV), mouse cytomegalovirus (MCMV), mouse rotavirus (EDIM), Kilham rat virus (KRV), Toolan's H-1 virus, Sendai virus (SeV, also know as murine parainfluenza virus type 1 or hemagglutinating virus of Japan (HVJ)), Parker's rat coronavirus (RCV or SDA), pseudorabies virus (PRV), reoviruses, Cache Valley virus, bovine viral diarrhoea virus, bovine parainfluenza virus type 3, bovine respiratory syncytial virus, bovine adenoviruses, bovine parvoviruses, bovine herpesvirus 1 (infectious bovine rhinotracheitis virus), other bovine herpesviruses, bovine reovirus, rabies virus, bluetongue viruses, bovine polyoma virus, bovine circovirus, and orthopoxviruses other than vaccinia, pseudocowpox virus (a widespread parapoxvirus that can infect humans), papillomavirus, herpesviruses, or leporipoxviruses.

Other embodiments target human-origin adventitious agents including HIV-1 and HIV-2; human T cell lymphotropic virus type I (HTLV-I) and HTLV-II; human hepatitis A, B, and C viruses; human cytomegalovirus; Epstein Barr virus (EBV or HHV-4); human herpesviruses 6, 7, and 8; human parvovirus B19; reoviruses; polyoma (JC/BK) viruses; SV40 virus; human coronaviruses; human papillomaviruses; influenza A, B, and C viruses; human enteroviruses; human parainfluenza viruses; and human respiratory syncytial virus.

Yet other embodiments of the present invention target host cell surface receptors or intracellular proteins to which endogenous, latent, or adventitious virus bind or which are required for viral replication. For example, in a particular embodiment, the target gene is a CHO cell MVM receptor gene, such as a gene associated with cellular sialic acid production.

In addition to the target genes associated with sialic acid, as described herein, yield and/or qualities of an immunogenic agent can be optimized by targeting genes associated with glycosylation in the host cell.

The hamster Gale gene encodes UDP-galactose-4-epimerase, e.g., CHO Gale transcript SEQ ID NO:5564, and can be targeted a RNA effector molecule comprising a sense strand and an antisense strand, one of which comprises at least 16 contiguous nucleotides (e.g., 17 nucleotides, 18 nucleotides, or 19 nucleotides) of the nucleotide sequence selected from the group consisting of SEQ ID NOs:1888656-1889007. In one embodiment, the antisense strand comprises at least 16 contiguous nucleotides of the nucleotide sequence selected from the group consisting of SEQ ID NOs:1888656-1889007. In another embodiment, one strand comprises at least 17 contiguous nucleotides of the nucleotide sequence selected from the group consisting of SEQ ID NOs:1888656-1889007. In another embodiment, one strand comprises at least 18 contiguous nucleotides of the nucleotide sequence selected from the group consisting of SEQ ID NOs:1888656-1889007. In another embodiment, one strand comprises at least 19 contiguous nucleotides of the nucleotide sequence selected from the group consisting of SEQ ID NOs:1888656-1889007. In a particular embodiment, the antisense strand comprises sequence of SEQ ID NOs:1888656-1889007, and further comprises at least one deoxyribonucleotide. In another particular embodiment, the antisense strand comprises sequence of SEQ ID NOs:1888656-1889007, and further comprises at least two deoxyribonucleotides. In another particular embodiment, the antisense strand comprises sequence of SEQ ID NOs:1888656-1889007, and further comprises at least two deoxythymidine residues. This enzyme enables the cell to process galactose by converting it to glucose, and vice versa.

UDP-galactose is used to build galactose-containing proteins and fats, which play critical roles in chemical signaling, building cellular structures, transporting molecules, and producing energy. Hamster GDP-mannose 4,6-dehydratase (GMDS) and can be targeted a RNA effector molecule comprising a sense strand and an antisense strand, one of which comprises at least 16 contiguous nucleotides (e.g., 17 nucleotides, 18 nucleotides, or 19 nucleotides) of the nucleotide sequence selected from the group consisting of SEQ ID NOs: 3152754-3152793. In one embodiment, the antisense strand comprises at least 16 contiguous nucleotides of the nucleotide sequence selected from the group consisting of SEQ ID NOs: 3152754-3152793. In another embodiment, one strand comprises at least 17 contiguous nucleotides of the nucleotide sequence selected from the group consisting of SEQ ID NOs: 3152754-3152793. In another embodiment, one strand comprises at least 18 contiguous nucleotides of the nucleotide sequence selected from the group consisting of SEQ ID NOs: 3152754-3152793. In another embodiment, one strand comprises at least 19 contiguous nucleotides of the nucleotide sequence selected from the group consisting of SEQ ID NOs: 3152754-3152793. In a particular embodiment, the antisense strand comprises sequence of SEQ ID NOs: 3152754-3152793, and further comprises at least one deoxyribonucleotide. In another particular embodiment, the antisense strand comprises sequence of SEQ ID NOs: 3152754-3152793, and further comprises at least two deoxyribonucleotides. In another particular embodiment, the antisense strand comprises sequence of SEQ ID NOs:3152754-3152793, and further comprises at least two deoxythymidine residues.

In various embodiments, the immunogenic agent is a polypeptide. The polypeptide can be a recombinant polypeptide or a polypeptide endogenous to the host cell. In some embodiments, the polypeptide is an antigen, a glycoprotein, a receptor, membrane protein, immune effector, binding protein, oncoprotein or proto-oncoprotein, or structural protein. In some embodiments, the polypeptide immunogenic agent is a vaccine or the immunogenic agent can be used in a vaccine.

The method of the invention also can include the steps of monitoring the growth, production and activation levels of the host cell culture, and as well as for varying the conditions of the host cell culture to maximize the growth, production and activation levels of the host cells and desired product, and for harvesting the immunogenic agent from the cell or culture, preparing a formulation with the harvested immunogenic agent, and for the treatment and/or the prevention of a disease by administering to a subject in need thereof a formulation obtained by the method.

In one embodiment, the host cell is administered a plurality of different RNA effector molecules to modulate expression of multiple target genes. The RNA effector molecules can be administered at different times or simultaneously, at the same frequency or different frequencies, at the same concentration or at different concentrations.

In another embodiment, the invention provides a composition for enhancing production of an immunogenic agent in a host cell by modulating the expression of a target gene in the cell. The composition typically includes one or more oligonuceotides, such as RNA effector molecules described herein, and a suitable carrier or delivery vehicle.

In additional embodiments, the target gene encodes a protein that affects a physiological process of the host cell. In various embodiments, the physiological process is apoptosis, cellular immunity, cell cycle progression, carbon metabolism or transport, lactate formation, or RNAi uptake and/or efficacy.

More specifically, in some embodiments the second target gene is a gene associated with host cell immune response, and the target gene encodes the host cell target selected from the group consisting of TLR3, TLR7, TLR21, RIG-1, LPGP2, RIG 1-like receptors, TRIM25, IFN-α, IFN-β, IFN-γ, MAVS, IFNAR1, IFNR2, STAT-1, STAT-2, STAT-3, STAT-4, JAK-1, JAK-2, JAK-3, IRF1, IRF2, IRF3, IRF4, IRF5, IRF6 IRF7, IRF8, IRF 9, IRF10, 2′,5′ oligoadenylate synthetase, RNaseL, dsRNA-dPKR, Mx, IFITM1, IFITM2, IFITM3, Proinflammatory cytokines, MYD88, TRIF, PKR, and a regulatory region of any of the foregoing.

In other specific embodiments, the second target gene is a gene associated with host cell viability, growth or cell cycle, and the target gene encodes the host cell target selected from the group consisting of Bax, Bak, LDHA, LDHB, BIK, BAD, BIM, HRK, BCLG, HR, NOXA, PUMA, BOK, BOO, BCLB, CASP2, CASP3, CASP6, CASP7, CASP8, CASP9, CASP10, BCL2, p53, APAF1, HSP70, TRAIL, BCL2L1, BCL2L13, BCL2L14, FASLG, DPF2, AIFM2, AIFM3, STK17A, APITD1, SIVA1, FAS, TGFβ2, TGFBR1, LOC378902, or BCL2A1, PUSL1, TPST1, WDR33, Nod2, MCT4, ACRC, AMELY, ATCAY, ANP32B, DEFA3, DHRS10, DOCK4, FAM106A, FKBP1B, IRF3, KBTBD8, KIAA0753, LPGAT1, MSMB, NFS1, NPIP, NPM3, SCGB2A1, SERPINB7, SLC16A4, SPTBN4, TMEM146, CDKN1B, CDKN2A, FOXO1, PTEN, FN1, CSKN2B, a miRNA antagonist, host sialidase, NEU2 sialidase 2, NEU3 sialidase 3, Dicer, ISRE, B4GalT1, B4GalT6, Cmas, Gne, SLC35A1, and a regulatory region of any of the foregoing.

In one aspect, the methods described herein relate to a method for improving the viability of a mammalian cell in culture, comprising: (a) contacting the cell with a plurality of different RNA effector molecules that permit inhibition of expression of Bax, Bak, and LDH; and (b) maintaining the cell for a time sufficient to inhibit expression of Bax, Bak, and LDH; wherein the inhibition of expression improves viability of the mammalian cell. In one embodiment of this aspect, the RNA effector molecule targeting BAX comprises a sense strand, and wherein at least one strand comprises at least 16 contiguous nucleotides (e.g., at least 17, at least 18, at least 19 nucleotides etc) of an oligonucleotide having a sequence selected from the group consisting of SEQ ID NOs:3152412-3152539, NOs:3152794-3152803, NOs:3023234-3023515, NOs:3154393-3154413, NOs:3154414-3154434, NOs:3154923-3154970, and NOs:3154971-3155018. In another embodiment of this aspect, the RNA effector molecule targeting BAK comprises a sense strand, and wherein at least one strand comprises at least 16 contiguous nucleotides (e.g., at least 17, at least 18, at least 19 nucleotides etc) of an oligonucleotide having a sequence selected from the group consisting of SEQ ID NOs:3152412-3152475, NOs:3152804-3152813, NOs:2259855-2260161, NOs:3154393-3154413, NOs:3154414-3154434, NOs:3154827-3154874, NOs:3154875-3154922 and sequences listed in Table 22. In another embodiment of this aspect, the RNA effector molecule targeting LDH comprises a sense strand, and wherein at least one strand comprises at least 16 contiguous nucleotides (e.g., at least 17, at least 18, at least 19 nucleotides etc) of an oligonucleotide having a sequence selected from the group consisting of SEQ ID NOs:3152540-3152603, NOs:3152814-3152823, NOs:1297283-1297604, NOs:3154553-3154578, NOs:3154579-3154604, NOs:3155589-3155635, and NOs:3155636-3155682.

In one aspect, the methods described herein provide a method for producing an immunogenic agent in a large scale host cell culture, comprising: (a) contacting a host cell in a large scale host cell culture with at least a first RNA effector molecule, a portion of which is complementary to at least one target gene of a host cell, (b) maintaining the host cell culture for a time sufficient to modulate expression of the at least one first target gene, wherein the modulation of expression improves production of an immunogenic agent in the host cell; (c) isolating the immunogenic agent from the host cell; wherein the large scale host cell culture is at least 1 Liter in size, and wherein the host cell is contacted with at least a first RNA effector molecule by addition of the RNA effector molecule to a culture medium of the large scale host cell culture such that the target gene expression is inhibited transiently.

Also provided herein in another aspect, are methods for producing an immunogenic agent in a large scale host cell culture, comprising: (a) contacting a host cell in a large scale host cell culture with at least a first RNA effector molecule, a portion of which is complementary to at least one target gene of a host cell, (b) maintaining the host cell culture for a time sufficient to modulate expression of the at least one first target gene, wherein the modulation of expression improves production of an immunogenic agent in the host cell; and (c) isolating the immunogenic agent from the host cell; wherein the host cell is contacted with at least a first RNA effector molecule by addition of the RNA effector molecule to a culture medium of the large scale host cell culture multiple times throughout production of the immunogenic agent such that the target gene expression is inhibited transiently.

In one embodiment of the aspects described herein, the host cell is contacted with the plurality of RNA effector molecules by addition of the RNA effector molecule to a culture medium of the large scale host cell culture such that the target gene expression is inhibited transiently.

In one embodiment of the aspects described herein, the host cell in the large scale host cell culture is contacted with a plurality of RNA effector molecules, wherein the plurality of RNA effector molecules modulate expression of at least one target gene, at least two target genes, or a plurality of target genes.

In another embodiment of the aspects described herein, the RNA effector molecule, or plurality of RNA effector molecules, comprises a double-stranded ribonucleic acid (dsRNA), wherein said dsRNA comprises at least two sequences that are complementary to each other and wherein a sense strand comprises a first sequence and an antisense strand comprises a second sequence comprising a region of complementarity which is substantially complementary to at least part of a target gene, and wherein said region of complementarity is 10 to 30 nucleotides in length.

In another embodiment of the aspects described herein, the contacting step is performed by continuous infusion of the RNA effector molecule, or plurality of RNA effector molecules, into the culture medium used for maintaining the host cell culture to produce the immunogenic agent.

In another embodiment of the aspects described herein, the modulation of expression is inhibition of expression, and wherein the inhibition is a partial inhibition.

In another embodiment of the aspects described herein, the partial inhibition is no greater than a percent inhibition selected from the group consisting of 10%, 15%, 20%, 25%, 30%, 35%, 40%, 45%, 50%, 55%, 60%, 65%, 70%, 75%, 80%, and 85%.

In another embodiment of the aspects described herein, the RNA effector molecule is contacted at a concentration of less than 100 nM.

In another embodiment of the aspects described herein, the RNA effector molecule is contacted at a concentration of less than 50 nM.

In some embodiments, at least one RNA effector molecule, a portion of which is complementary to the target gene, is a corresponding siRNA that comprises an antisense strand comprising at least 16 contiguous nucleotides of a nucleotide sequence, wherein the nucleotide sequence (SEQ ID NO) is referred to herein.

Also provided herein are compositions useful for enhancing production of an immunogenic agent. In one aspect, a composition is provided that comprises at least one RNA effector molecule, a portion of which is complementary to at least one target gene of a host cell, and a cell medium suitable for culturing the host cell, wherein the RNA effector molecule is capable of modulating expression of the target gene and the modulation of expression enhances production of an immunogenic agent, wherein the at least one RNA effector molecule is an siRNA that comprises an antisense strand comprising at least 16 contiguous nucleotides of a nucleotide sequence (SEQ ID NO) referred to herein.

Another aspect described herein provides a kit for enhancing production of an immunogenic agent by a cultured cell, comprising: (a) a substrate comprising one or more assay surfaces suitable for culturing the cell under conditions in which the immunogenic agent is produced; (b) one or more RNA effector molecules, wherein at least a portion of each RNA effector molecule is complementary to a target gene; and (c) a reagent for detecting the immunogenic agent or production thereof by the cell, wherein the one or more RNA effector molecules is an siRNA comprising an antisense strand that comprises at least 16 contiguous nucleotides of the nucleotide sequence (SEQ ID NO) referred to herein.

Also provided herein is a kit for optimizing production of an immunogenic agent by cultured cells, comprising: (a) a microarray substrate comprising a plurality of assay surfaces, the assay surfaces being suitable for culturing the cells under conditions in which the immunogenic agent is produced; (b) one or more RNA effector molecules, wherein at least a portion of each RNA effector molecule is complementary to a target gene; and (c) a reagent for detecting the effect of the one or more RNA effector molecules on production of the immunogenic agent, wherein the one or more RNA effector molecules is an siRNA comprising an antisense strand that comprises at least 16 contiguous nucleotides of a nucleotide sequence (SEQ ID NO) referred to herein.

In one embodiment, the invention provides for a host cell that contains at least one RNA effector molecule provided herein. The host cell can be derived from an insect, amphibian, fish, reptile, bird, mammal, or human, or can be a hybridoma cell. For example, the cell can be a human Namalwa Burkitt lymphoma cell (BLcl-kar-Namalwa), baby hamster kidney fibroblast (BHK), CHO cell, Murine myeloma cell (e.g., NS0, SP2/0), hybridoma cell, human embryonic kidney cell (293 HEK), human retina-derived cell (PER.C6® cells), insect cell line (Sf9, derived from pupal ovarian tissue of Spodoptera frugiperda; or Hi-5, derived from Trichoplusia ni egg cell homogenates), Madin-Darby canine kidney cell (MDCK), primary mouse brain cells or tissue, primary calf lymph cells or tissue, primary monkey kidney cell, embryonated chicken egg, primary chicken embryo fibroblast (CEF), Rhesus fetal lung cell (FRhL-2), Human fetal lung cell (WI-38, MRC-5), African green monkey kidney epithelial cell (e.g., Vero, CV-1), Rhesus monkey kidney cell (LLC-MK2), or yeast cell. In a particular embodiment, the cell is a MDCK cell.

Embodiments also provide compositions and methods for producing an immunogenic agent from a host cell, particularly from CHO cell, the methods comprising contacting the cell with a RNA effector molecule, such as one or more siRNA molecules targeting the CHO transcriptome transcripts, a portion of which is complementary to a target transcript, maintaining the cell in a bioreactor for a time sufficient to modulate expression of the target gene, wherein the modulation enhances production of the immunogenic agent from the cell, and isolating the immunogenic agent from the cell.

An advantage of the present invention is the ability to substantially increase the yield and/or purity of the immunogenic agents produced by the host cells, and thereby reduce production costs, or to significantly reduce development times. Improved manufacturing logistics have the follow-on effect of enhancing quality, as well as expanding immunogenic agent product supply.

The details of one or more embodiments of the invention are set forth in the description below. Other features, objects, and advantages of the invention will be apparent from the description and the drawings, and from the claim.

### DESCRIPTION OF THE DRAWINGS

FIGS. 1A and 1B: FIG. 1A is am immunoblot labeling the Bax protein in day 2 CHO—S cells. The expression of Bax correlates with the decrease in viability over time in CHO—S cell cultures. The expression of Bax correlates with the decrease in viability over time in CHO—S cell cultures. FIG. 1B is a graph depicting the growth curve for CHO—S cells showing cell viability, total cell number, and proportion of viable cells as a function of days in cell culture. Viability decreases sharply around day 6.

FIGS. 2A and 2B are graphs depicting concentration-dependent inhibition of expression of Bak (FIG. 2B) and Bax (FIG. 2A) in CHO cells by RNA effector molecules against hamster Bak and Bax genes (Tables 3 and 4, respectively). Each of the tested RNA effector molecules inhibited expression with an 1050 in the sub-nanomolar range, except for RNA effector molecule B2 against Bax, which inhibited expression with an 1050 in the low nanomolar range.

FIG. 3 is a graph showing concentration-dependent inhibition of expression of LDH (measured as LDH activity) in CHO cells by RNA effector molecules against the hamster lactate dehydrogenase (LDH) gene. Each of the tested RNA effector molecules inhibited expression with an 1050 in the sub-nanomolar range.

FIGS. 4A to 4D: RNA effector molecules against hamster lactate dehydrogenase (LDH) decrease levels of LDH-A mRNA (FIG. 4A), protein (FIG. 4B), and activity (FIG. 4C) in C2, C16 and C36 CHO cell lines relative to control cells. Inhibition of LDH significantly enhances productivity of the CHO cell lines (FIG. 4D).

FIG. 5A to 5B: FIG. 5A is a bar graph and FIG. 5B is a line graph, each showing the effect of RNA effector molecules against Bax/Bak and LDH on the viability of cultured CHO cells. siRNA (1 nM) were added to cultured cells at O-hr, 48-hr and 96-hr timepoints (arrows on curve) and cell viability was measured as the integral cell area (ICA) at day 5 (graph) and over time (curve). Control cells were treated with Stealth siRNA (scrambled control). Cells treated with siRNA against Bax/Bak and LDH exhibited enhanced viability relative to control cells at all time points measured.

FIG. 6 is a graph depicting that the addition of Bax/Bak/LDH siRNAs increases viable CHO cell density by at least 90%. Control cell (▪) and treated cell (▴) densities were measured daily until cell viability reached 50%. Integral cell areas (IGA) were determined (inset; control vs. Bax/Bak/LDH siRNA-treated). Arrows on x-axis indicate siRNA dosing days or nutrient feed days.

FIG. 7 is a graph depicting that the addition of Bax/Bak/LDH siRNAs increases percent viability of CHO by at least 50%. Percent viability of control cells (▪) and cells treated with Bax/Bak/LDH siRNAs (▴) were determined using Trypan Blue. The rate of apoptotic cell death was determined by measuring the slopes of each sample from day-5 until day-12 (inset; control vs. Bax/Bak/LDH siRNA-treated). Arrows on x-axis indicate siRNA dosing days.

FIG. 8 is a graph depicting that LDH enzyme activity is decreased in Bax/Bak/LDH siRNA-treated cells. Daily LDH activities were monitored in control-treated (▪) and Bax/Bak/LDH siRNA-treated cells (▴). Arrows on x-axis indicate siRNA dosing days.

FIG. 9 is a graph showing that lactate levels are lower in Bax/Bak/LDH siRNA-treated cell culture media compared to the control-treated cell media. Lactate levels in culture media were monitored daily in control siRNA-treated (▪) and Bax/Bak/LDH siRNA-treated (▴) cell cultures. Arrows on x-axis indicate siRNA dosing days.

FIG. 10 is a graph showing that glucose consumption in control siRNA-treated cells decreases following day 7 of the growth curve. Glucose levels from the Bax/Bak/LDH siRNA-treated cell media (▴) is significantly lower than the control siRNA-treated cell media (▪). Arrows along x-axis indicate nutrient feed days.

FIG. 11 is a graph showing that Bax/Bak/LDH siRNA-treated CHO cells have decreased Caspase 3 activity following log phase growth compared to control. Bax/Bak/LDH siRNA-treated cells demonstrate similar Caspase 3 activity to the control-siRNA-treated cells prior to day 6 but the following time points show higher Caspase activity in the control cells. A ratio (▴) between Caspase 3 activity in the Bax/Bak/LDH siRNA-treated cells and in control-treated cells shows a biphasic activity response.

FIG. 12 is a graph showing the percent inhibition of mRNA level following Bax, Bak, and LDH siRNA addition.

FIG. 13 is a graph depicting that Bax/Bak/LDH siRNA decreases CHO cell apoptosis death rate by ˜300%.

FIG. 14 is a graph depicting the viability and cell density of cell treated with Bax/Bak siRNA (1 nM each) compared to a control FITC-siRNA (1 nM).

FIGS. 15A and 15B: FIG. 15A is a graph depicting the cell density and viability ratio of cells treated with siRNA targeting Bax/Bak/LDH compared to control treated cells. FIG. 15B shows that Bax/Bak/LDH siRNA improves both CHO cell density and viability in a large scale, 1 L bioreactor.

FIG. 16 shows a diagrammatic view of a computer system according to one embodiment of the invention.

FIG. 17 shows a diagrammatic view of a computer system according to an alternative embodiment of the invention.

FIG. 18 presents a diagram of the data structures according to one embodiment of the invention.

FIG. 19 shows a flow diagram of a method according to one embodiment of the invention.

FIG. 20 is a graph showing expression levels (fluorometric units, y-axis) of GFP over time in days (X-axis) in control DG44 CHO cells treated with lipid RNAiMax and no siRNAs, at temperatures of 37° C. and 28° C., i.e. lipid treated control.

FIG. 21 is a graph showing expression levels (fluorometric units, y-axis) of GFP over time in days (X-axis) in control DG44 CHO cells not treated with lipid RNAiMax or siRNAs, at temperatures of 37° C. and 28° C., i.e untreated controls.

FIGS. 22A-22C are graphs showing the % inhibition of GFP expression (y-axis) in DG44 CHO cells by transiently transfected siRNAs against GFP at 37° C. and 28° C. over time in days (x-axis). FIG. 22A, 0.1 nM siRNA. FIG. 22B, 1.0 nM siRNA. FIG. 22C, 10 nM siRNA.

FIG. 23 is a bar graph showing relative % GFP signal knockdown (y-axis) using 9 uptake enhancing formulations compared to Lipofectamine RNAiMax, see Table 19, for the 9 formulations depicted on the x-axis.

FIG. 24 is a bar graph showing LDH activity (y axis) using K8 (formulation 4) at various concentrations was effective as an uptake enhancer of siRNA against LDH in DG44 cells in a 250 mL shake flask.

FIG. 25 is a bar graph showing LDH activity (y axis) using K8 (formulation 4), L8, and P8 formulations at various concentrations were effective as uptake enhancers of siRNA against LDH in DG44 in suspension.

FIGS. 26A-26B are graph showing cell density (FIG. 26A) or % cell viability (FIG. 26B) over time in suspension CHO cell 50 mL shake flasks using P8 formulation or commercial formulation RNAiMax at the recommended concentration. Lipid formulations were dosed onto cells at day 0.

FIG. 30 is a graph that shows when sing the P8 NDL an siRNA directed against Lactate Dehydrogenase (LDH) achieves 80%-90% knockdown of LDH activity for 6 days with a single 1 nM dose in a 1 L bioreactor.

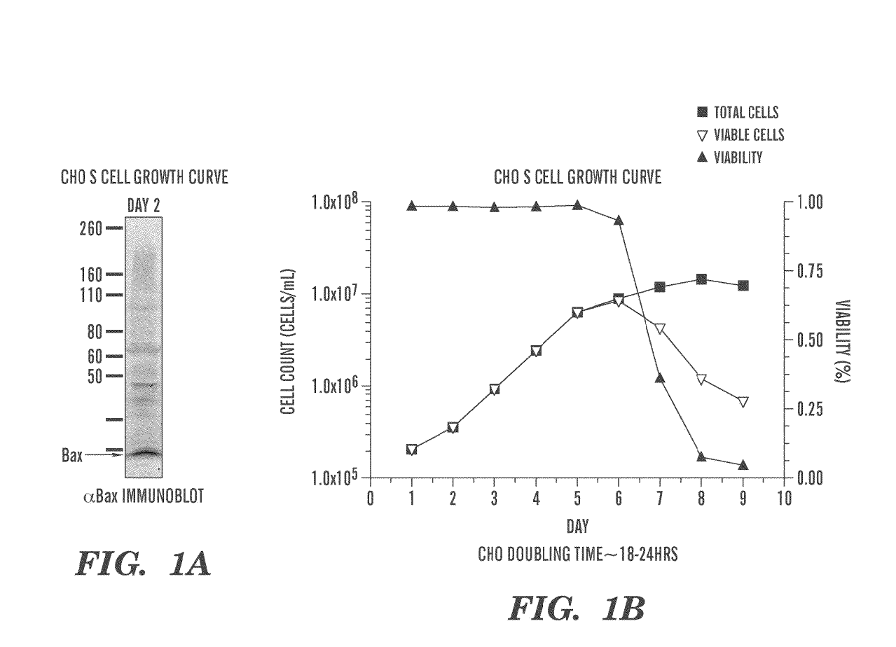

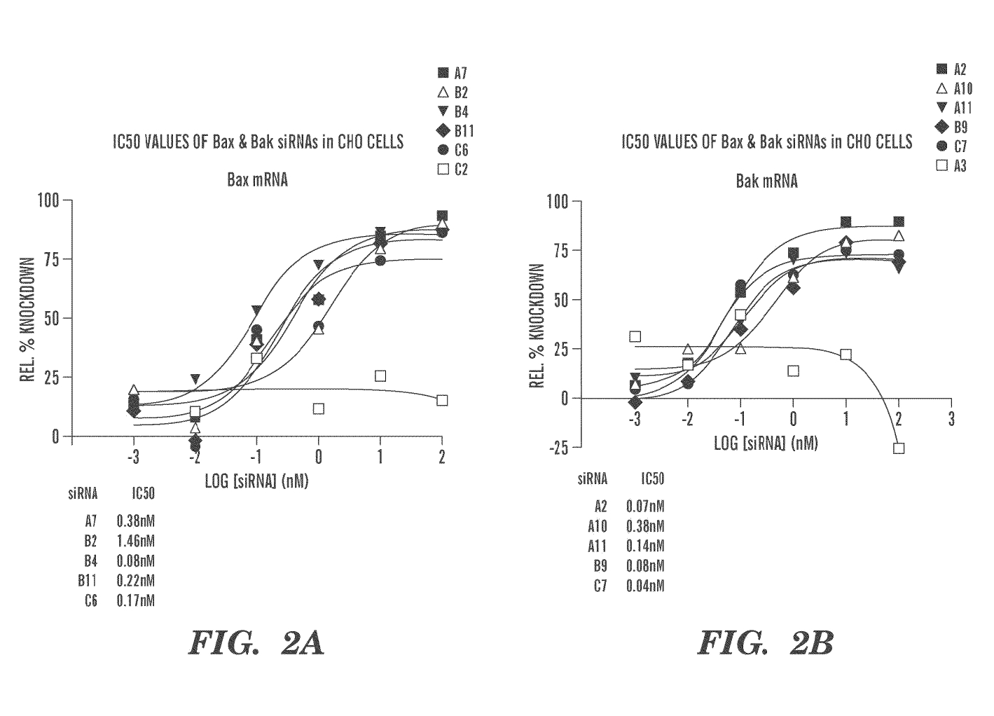

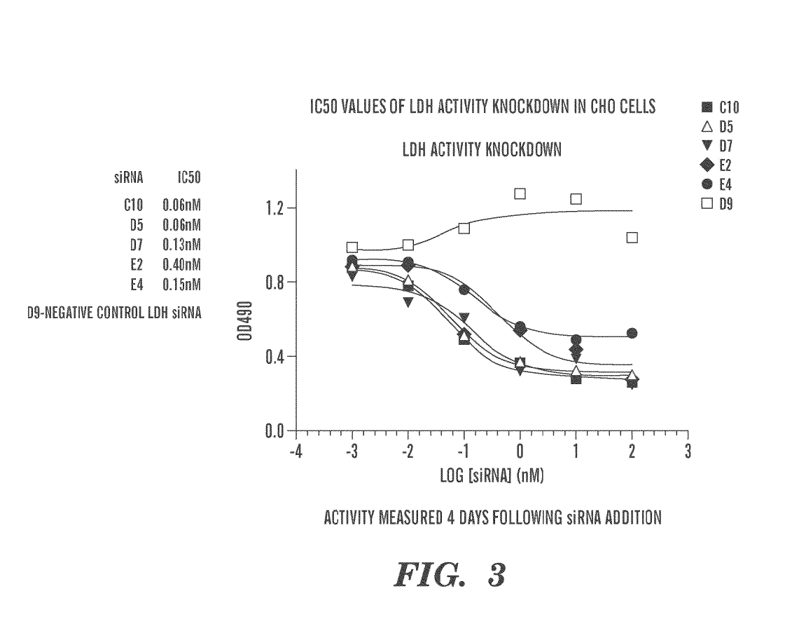

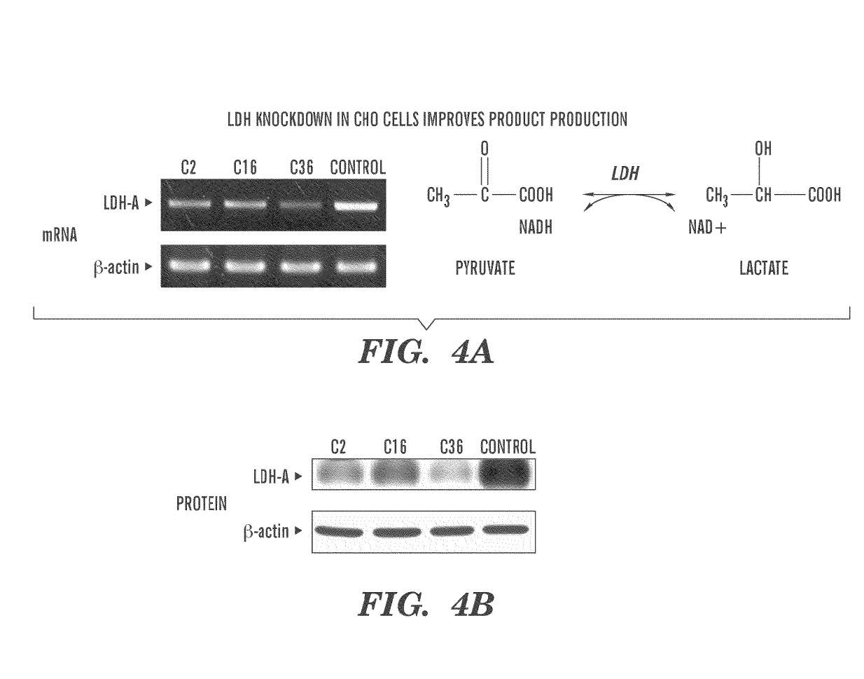

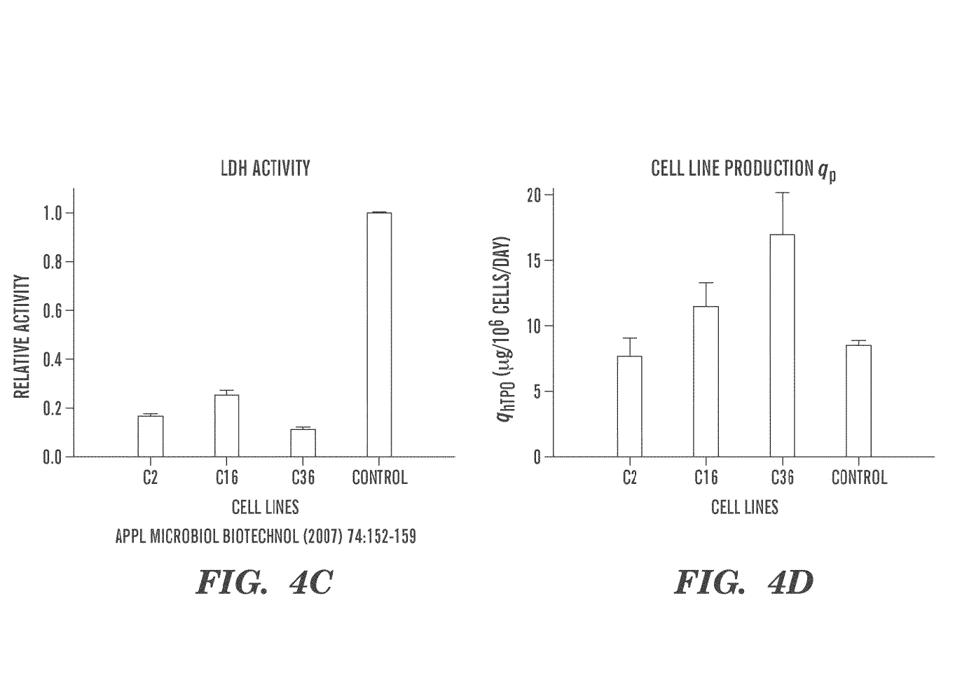

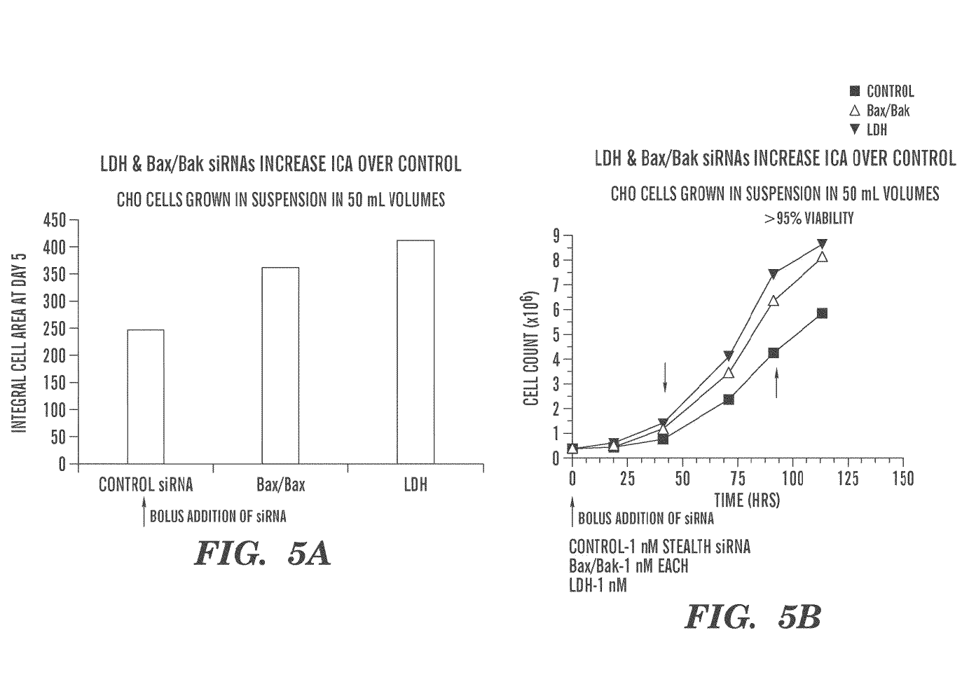

FIG. 28 is a graph that shows the results of a single dose of a 1 nM LDH siRNA formulated with P8 lipid on viable cell density and % LDH activity over an elapsed time of 6 days in 3 L and 40 L cultures.

FIG. 29 is a graph showing viable cell density and % viability (y-axis) over time in days after transfection of 40 L of DG44 cell culture using P8 as the transfection reagent.

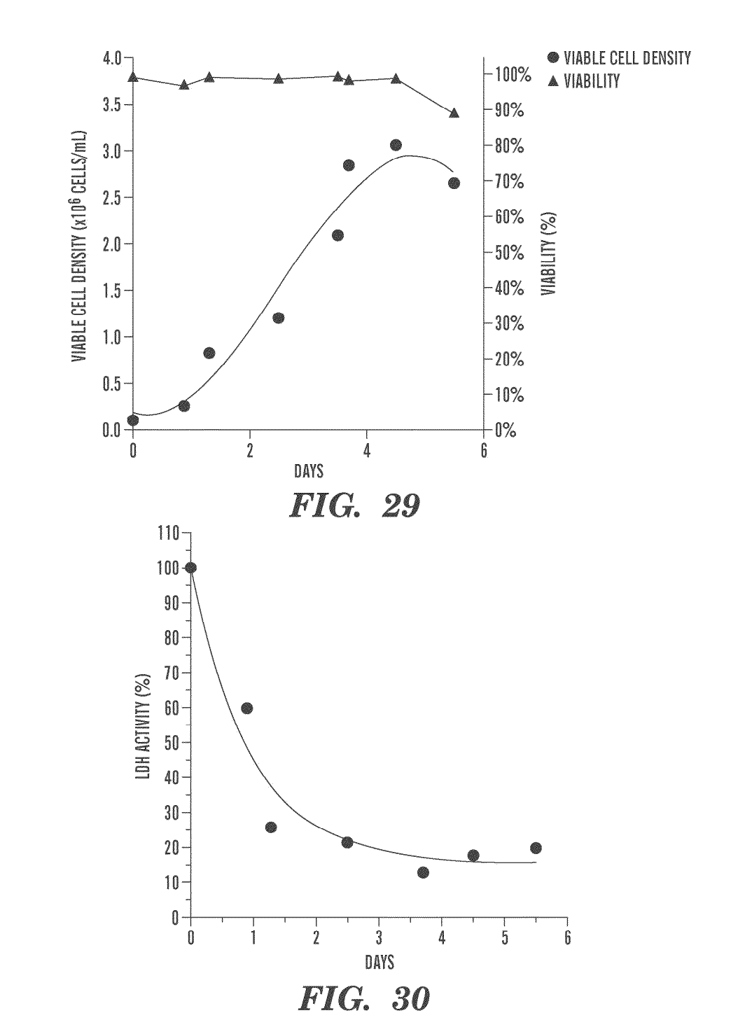

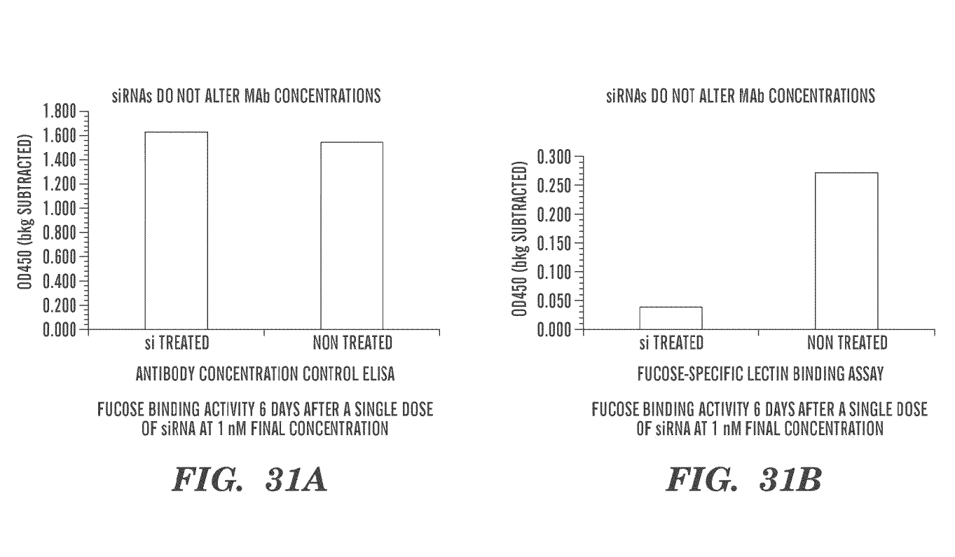

FIG. 30 is a graph showing reduction in % LDH activity over time in 40 L of DG44 cell culture and a single dose of siRNA at day 0.

FIGS. 31A and 31B are bar graphs of antibodies prepared from control cells of cells contacted with dsRNA targeting the fucosyltransferase (FUT8) and GDP-mannose 4,6-dehydratase (GMDS) genes. FIG. 31A is a graph that shows the concentration of antibody produced by these cells; FIG. 31B is a graph that shows that antibodies produced from the FUT8 and GMDS dsRNA treated cells have >85% reduced binding to fucose-specific lectin.

### DETAILED DESCRIPTION

The present invention is not limited to the particular methodology, protocols, and compositions, etc., described herein, as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents, oligonucleotide sequences identified by gene identification numbers, and other publications identified herein are expressly incorporated by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although human gene symbols are typically designated by upper-case letters, in the present specification the use of either upper-case or lower-case gene symbols may be used interchangeably and include both human or non-human species. Thus, for example, a reference in the specification to the gene or gene target “lactate dehydrogenase A” as “LDHA” (“Ldha” or “LdhA”), includes human and/or non-human (e.g., avian, rodent, canine) genes and gene targets. In other words, the upper-case or lower-case letters in a particular gene symbol do not limit the scope of the gene or gene target to human or non-human species. All gene identification numbers provided herein (GeneID) are those of the National Center for Biotechnology Information “Entrez Gene” web site unless identified otherwise.

The invention provides methods for producing an immunogenic agent in a host cell, the methods including the steps of contacting the cell with at least one RNA effector molecule, a portion of which is complementary to at least a portion of a target gene, maintaining the cell for a time sufficient to modulate expression of the target gene, wherein the modulation enhances production of the immunogenic agent, and recovering the immunogenic agent from the cell. The description provided herein discloses how to make and use RNA effector molecules to produce a immunogenic agent in a host cell according to methods provided herein. Also disclosed are cell culture reagents and compositions comprising the RNA effector molecules and kits for carrying out the disclosed methods.

### I. DEFINITIONS

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used herein, “immunogenic agent” refers to an agent used to stimulate the immune system of a subject, so that one or more functions of the immune system are increased and directed towards the immunogenic agent. An antigen or immunogen is intended to mean a molecule containing one or more epitopes that can stimulate a host immune system to make a secretory, humoral and/or cellular immune response specific to that antigen. Immunogenic agents can be used in the production of antibodies, both isolated polyclonal antibodies and monoclonal antibodies, using techniques known in the art Immunogenic agents include vaccines.

As used herein, “vaccine” refers to an agent used to stimulate the immune system of a subject so that protection is provided against an antigen not recognized as a self-antigen by the subject's immune system Immunization refers to the process of inducing a high level of antibody and/or cellular immune response in a subject, that is directed against a pathogen or antigen to which the organism has been exposed. Vaccines and immunogenic agents as used herein, refer to a subject's immune system: the anatomical features and mechanisms by which a subject produces antibodies and/or cellular immune responses against an antigenic material that invades the subject's cells or extra-cellular fluids. In the case of antibody production, the antibody so produced can belong to any of the immunological classes, such as immunoglobulins, A, D, E, G, or M. Vaccines that stimulate production of immunoglobulin A (IgA) are of interest, because IgA is the principal immunoglobulin of the secretory system in warm-blooded animals. Vaccines are likely to produce a broad range of other immune responses in addition to IgA formation, for example cellular and humoral immunity. Immune responses to antigens are well-studied and reported widely. See, e.g., Elgert, IMMUNOL. (Wiley Liss, Inc., 1996); Stites et al., BASIC & CLIN. IMMUNOL., (7th Ed., Appleton & Lange, 1991). By contrast, the phrase “immune response of the host cell” refers to the responses of unicellular host organisms to the presence of foreign bodies.

In the context of this invention, the term “oligonucleotide” or “nucleic acid molecule” encompasses not only nucleic acid molecules as expressed or found in nature, but also analogs and derivatives of nucleic acids comprising one or more ribo- or deoxyribo-nucleotide/nucleoside analogs or derivatives as described herein or as known in the art. Such modified or substituted oligonucleotides are often used over native forms because of properties such as, for example, enhanced cellular uptake, increased stability in the presence of nucleases, and the like, discussed further herein. A “nucleoside” includes a nucleoside base and a ribose sugar, and a “nucleotide” is a nucleoside with one, two or three phosphate moieties. The terms “nucleoside” and “nucleotide” can be considered to be equivalent as used herein. An oligonucleotide can be modified in the nucleobase structure or in the ribose-phosphate backbone structure, e.g., as described herein, including the modification of a RNA nucleotide into a DNA nucleotide. The molecules comprising nucleoside analogs or derivatives must retain the ability to form a duplex.

As non-limiting examples, an oligonucleotide can also include at least one modified nucleoside including but not limited to a 2′-O-methyl modified nucleoside, a nucleoside comprising a 5′ phosphorothioate group, a terminal nucleoside linked to a cholesterol derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. Alternatively, an oligonucleotide can comprise at least two modified nucleosides, at least 3, at least 4, at least 5, at least 6, at least 7, at least 8, at least 9, at least 10, at least 15, at least 20, or more, up to the entire length of the oligonucleotide. The modifications need not be the same for each of such a plurality of modified nucleosides in an oligonucleotide. When RNA effector molecule is double stranded, each strand can be independently modified as to number, type and/or location of the modified nucleosides. In one embodiment, modified oligonucleotides contemplated for use in methods and compositions described herein are peptide nucleic acids (PNAs) that have the ability to form the required duplex structure and that permit or mediate the specific degradation of a target RNA via a RISC pathway.

The terms “ribonucleoside”, “ribonucleotide”, “nucleotide”, or “deoxyribonucleotide” can also refer to a modified nucleotide, as further detailed herein, or a surrogate replacement moiety. A ribonucleotide comprising a thymine base is also referred to as 5-methyl uridine and a deoxyribonucleotide comprising a uracil base is also referred to as deoxy-Uridine in the art. Guanine, cytosine, adenine, thymine and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

Similarly, the skilled artisan will recognize that the term “RNA molecule” or “ribonucleic acid molecule” encompasses not only RNA molecules as expressed or found in nature, but also analogs and derivatives of RNA comprising one or more ribonucleotide or ribonucleoside analogs or derivatives as described herein or as known in the art. The terms “ribonucleoside” and “ribonucleotide” can be considered to be equivalent as used herein. The RNA can be modified in the nucleobase structure or in the ribose-phosphate backbone structure, e.g., as described herein.

In one aspect, a RNA effector molecule can include a deoxyribonucleoside residue. In such an instance, a RNA effector molecule agent can comprise one or more deoxynucleosides, including, for example, a deoxynucleoside overhang(s), or one or more deoxynucleosides within the double stranded portion of a dsRNA.

In some embodiments, a plurality of RNA effector molecules is used to modulate expression of one or more target genes. A “plurality” refers to at least 2 or more RNA effector molecules e.g., 3, 4, 5, 6, 7, 8, 9, 10, 11, 12, 13, 14, 15, 16, 17, 18, 19, 20, 30, 40, 50, 60, 80, 100 RNA effector molecules or more. “Plurality” can also refer to at least 2 or more target genes, e.g., 2, 3, 4, 5, 6, 7, 8, 9, 10, 11, 12, 13, 14, 15, 16, 17, 18, 19, 20, 30, 40, 50, 60, 70, 80, 90, 100 target genes or more.

As used herein the term “contacting a host cell” refers to the treatment of a host cell with an agent such that the agent is introduced into the cell. Typically the host cell is in culture, e.g., using at least one RNA effector molecule (e.g., a siRNA), often prepared in a composition comprising a delivery agent that facilitates RNA effector uptake into the cell e.g., to contact the cell in culture by adding the composition to the culture medium. In one embodiment the host cell is contacted with a vector that encodes a RNA effector molecule, e.g. an integrating or non-integrating vector. In one embodiment the cell is contacted with a vector that encodes a RNA effector molecule prior to culturing the host cell for immunogenic agent production, e.g., by transfection or transduction.

In one embodiment contacting a host cell does not include contacting the host cell with a vector that encodes a RNA effector molecule. In one embodiment, contacting a host cell does not include contacting a host cell with a vector the encodes a RNA effector molecule prior to culturing the host cell for immunogenic agent production, i.e., the cell is contacted with a RNA effector molecule only in cell growth culture, e.g., added to the host cell culture during the process of producing an immunogenic agent. For example, some embodiments of the present invention provide for contacting a host cell with a RNA effector molecule (e.g., a dsRNA) occurs prior to, during or after the viral infection or vector inoculation to inhibit cellular and anti-viral processes that compromise the yield and quality of the immunogenic agent harvest. The step of contacting a host cell in culture with a RNA effector molecule(s) can be repeated more than once (e.g., twice, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100× or more). In one embodiment, the cell is contacted such that the target gene is modulated only transiently, e.g., by addition of a RNA effector molecule composition to the cell culture medium used for the production of an immunogenic agent where the presence of the RNA effector molecules dissipates over time, i.e., the RNA effector molecule is not constitutively expressed in the cell.

“Introducing into a cell”, when referring to a RNA effector molecule, means facilitating or effecting uptake or absorption into the cell, as is understood by those skilled in the art. Absorption or uptake of a RNA effector molecule can occur through unaided diffusive or active cellular processes, or by auxiliary agents or devices. For example, introducing into a cell means contacting a host cell with at least one RNA effector molecule, or means the treatment of a cell with at least one RNA effector molecule and an agent that facilitates or effects uptake or absorption into the cell, often prepared in a composition comprising the RNA effector molecule and delivery agent that facilitates RNA effector molecule uptake (e.g., a transfection reagent, an emulsion, a cationic lipid, a non-cationic lipid, a charged lipid, a liposome, an anionic lipid, a penetration enhancer, or a modification to the RNA effector molecule to attach, e.g., a ligand, a targeting moiety, a peptide, a lipophillic group etc.). In vitro introduction into a cell includes methods known in the art such as electroporation and lipofection. Further approaches are described herein below or known in the art.

As used herein, a “RNA effector composition” includes an effective amount of a RNA effector molecule and an acceptable carrier. As used herein, “effective amount” refers to that amount of a RNA effector molecule effective to produce an effect (e.g., modulatory effect) on a bioprocess for the production of an immunogenic agent. In one embodiment, the RNA effector composition comprises a reagent that facilitates RNA effector molecule uptake (e.g., a transfection reagent, an emulsion, a cationic lipid, a non-cationic lipid, a charged lipid, a liposome, an anionic lipid, a penetration enhancer, or a modification to the RNA effector molecule to attach e.g., a ligand, a targeting moiety, a peptide, a lipophillic group, etc.)

The term “acceptable carrier” refers to a carrier for administration of a RNA effector molecule to cultured cells. Such carriers include, but are not limited to, saline, buffered saline, dextrose, water, glycerol, ethanol, and combinations thereof. In one embodiment the term “acceptable carrier” specifically excludes cell culture medium.

The term “expression” as used herein is intended to mean the transcription to a RNA and/or translation to one or more polypeptides from a target gene coding for the sequence of the RNA and/or the polypeptide.

As used herein, “target gene” refers to a gene that encodes a protein that affects one or more aspects of the production of an immunogenic agent by a host cell, such that modulating expression of the gene enhances production of an immunogenic agent. Target genes can be derived from the host cell, endogenous to the host cell (present in the host cell genome), transgenes (gene constructs inserted at ectopic sites in the host cell genome), or derived from a pathogen (e.g., a virus, fungus or bacterium) that is capable of infecting the host cell or the subject who will use the immunogenic agent or derivatives thereof (e.g., humans). Additionally, in some embodiments, a “target gene” refers to a gene that regulates expression of a nucleic acid (i.e., non-encoding genes) that affects one or more aspects of the production of an immunogenic agent by a cell, such that modulating expression of the gene enhances production of the immunogenic agent.

By “target gene RNA” or “target RNA” is meant RNA transcribed from the target gene. Hence, a target gene can be a coding region, a promoter region, a 3′ untranslated region (3′-UTR), and/or a 5′-UTR of the target gene.

A target gene RNA that encodes a polypeptide is more commonly known as messenger RNA (mRNA). Target genes can be derived from the host cell, latent in the host cell, endogenous to the host cell (present in the host cell genome), transgenes (gene constructs inserted at ectopic sites in the host cell genome), or derived from a pathogen (e.g., a virus, fungus or bacterium) which is capable of infecting either the host cell or the subject who will use the an immunogenic agent or derivatives or products thereof. In some embodiments, the target gene encodes a protein that affects one or more aspects of post-translational modification, e.g., peptide glycosylation, by a host cell. For example, modulating expression of a gene encoding a protein involved in post-translational processing enhances production of a polypeptide comprising at least one terminal mannose.

In some embodiments, the target gene encodes a non-coding RNA (ncRNA), such as an untranslated region. As used herein, a ncRNA refers to a target gene RNA that is not translated into a protein. The ncRNA can also be referred to as non-protein-coding RNA (npcRNA), non-messenger RNA (nmRNA), small non-messenger RNA (snmRNA), and functional RNA (fRNA) in the art. The target gene from which a ncRNA is transcribed as the end product is also referred to as a RNA gene or ncRNA gene. ncRNA genes include highly abundant and functionally important RNAs such as transfer RNA (tRNA) and ribosomal RNA (rRNA), as well as RNAs such as snoRNAs, microRNAs, siRNAs, and piRNAs. As used herein, a RNA effector molecule is said to target within a particular site of a RNA transcript if the RNA effector molecule promotes cleavage of the transcript anywhere within that particular site.

In some embodiments, the target gene is an endogenous gene of the host cell. For example, the target gene can encode the immunogenic agent or a portion thereof when the immunogenic agent is a polypeptide. The target gene can also encode a host cell protein that directly or indirectly affects one or more aspects of the production of the immunogenic agent. Examples of target genes that affect the production of polypeptides include genes encoding proteins involved in the secretion, folding or post-translational modification of polypeptides (e.g., glycosylation, deamidation, disulfide bond formation, methionine oxidation, or pyroglutamation); genes encoding proteins that influence a property or phenotype of the host cell (e.g., growth, viability, cellular pH, cell cycle progression, apoptosis, carbon metabolism or transport, lactate formation, cytoskeletal structure (e.g., actin dynamics), susceptibility to viral infection or RNAi uptake, activity, or efficacy); and genes encoding proteins that impair the production of an immunogenic agent by the host cell (e.g., a protein that binds or co-purifies with the immunogenic agent).

In some embodiments, the target gene encodes a host cell protein that indirectly affects the production of the immunogenic agent such that inhibiting expression of the target gene enhances production of the immunogenic agent. For example, the target gene can encode an abundantly expressed host cell protein that does not directly influence production of the immunogenic agent, but indirectly decreases its production, for example by utilizing cellular resources that could otherwise enhance production of the immunogenic agent. Target genes are discussed in more detail herein.

The term “modulates expression of” and the like, in so far as it refers to a target gene, herein refers to the modulation of expression of a target gene, as manifested by a change (e.g., an increase or a decrease) in the amount of target gene mRNA that can be isolated from or detected in a first cell or group of cells in which a target gene is transcribed and that has or have been treated such that the expression of a target gene is modulated, as compared to a second cell or group of cells substantially identical to the first cell or group of cells but that has or have not been so treated (control cells). The degree of modulation can be expressed in terms of:

$$\frac{( mRNA in control cells ) - ( mRNA in treated cells )}{( mRNA in control cells )} \cdot 100 \%$$

Alternatively, the degree of modulation can be given in terms of a parameter that is functionally linked to target gene expression, e.g., the amount of protein encoded by a target gene, or the number of cells displaying a certain phenotype, e.g., stabilization of microtubules. In principle, target gene modulation can be determined in any host cell expressing the target gene, either constitutively or by genomic engineering, and by any appropriate assay known in the art.

For example, in certain instances, expression of a target gene is inhibited. For example, expression of a target gene is inhibited by at least about 10%, 15%, 20%, 25%, 30%, 35%, 40%, 45%, or 50% by administration of a RNA effector molecule provided herein. In some embodiments, a target gene is inhibited by at least about 60%, 70%, or 80% by administration of a RNA effector molecule. In some embodiments, a target gene is inhibited by at least about 85%, 90%, or 95% or more by administration of a RNA effector molecule as described herein. In other instances, expression of a target gene is activated by at least about 10%, 20%, 25%, 50%, 100%, 200%, 400% or more by administration of a RNA effector molecule provided herein. In some embodiments, the modulation of expression is a partial inhibition. In some aspects, the partial inhibition is no greater than a percent inhibition selected from the group consisting of: 10%, 15%, 20%, 25%, 30%, 35%, 40%, 45%, 50%, 55%, 60%, 65%, 70%, 75%, 80%, and 85%.

As used herein, the term “RNA effector molecule” refers to an oligonucleotide agent capable of modulating the expression of a target gene, as defined herein, within a host cell, or a oligonucleotide agent capable of forming such an oligonucleotide, optionally, within a host cell (i.e., upon being introduced into a host cell). A portion of a RNA effector molecule is substantially complementary to at least a portion of the target gene, such as the coding region, the promoter region, the 3′ untranslated region (3′-UTR), and/or the 5′-UTR of the target gene.

The RNA effector molecules described herein generally have a first strand and a second strand, one of which is substantially complementary to at least a portion of the target gene and modulate expression of target genes by one or more of a variety of mechanisms, including but not limited to, Argonaute-mediated post-transcriptional cleavage of target gene mRNA transcripts (sometimes referred to in the art as RNAi) and/or other pre-transcriptional and pre-translational mechanisms.

RNA effector molecules can comprise a single strand or more than one strand, and can include, e.g., double stranded RNA (dsRNA), microRNA (miRNA), antisense RNA, promoter-directed RNA (pdRNA), Piwi-interacting RNA (piRNA), expressed interfering RNA (eiRNA), short hairpin RNA (shRNA), antagomirs, decoy RNA, DNA, plasmids, and aptamers. The RNA effector molecule can be single-stranded or double-stranded. A single-stranded RNA effector molecule can have double-stranded regions and a double-stranded RNA effector can have single-stranded regions.

The term “portion”, when used in reference to an oligonucleotide (e.g., a RNA effector molecule), refers to a portion of a RNA effector molecule having a desired length to effect complementary binding to a region of a target gene, or a desired length of a duplex region. For example, a “portion” or “region” refers to a nucleic acid sequence of at least 3, at least 4, at least 5, at least 6, at least 7, at least 8, at least 9, at least 10 or more nucleotides up to one nucleotide shorter than the entire RNA effector molecule. In some embodiments, the “region” or “portion” when used in reference to a RNA effector molecule includes nucleic acid sequence one nucleotide shorter than the entire nucleic acid sequence of a strand of a RNA effector molecule. One of skill in the art can vary the length of the “portion” that is complementary to the target gene or arranged in a duplex, such that a RNA effector molecule having desired characteristics (e.g., inhibition of a target gene or stability) is produced. Although not bound by theory, RNA effector molecules provided herein can modulate expression of target genes by one or more of a variety of mechanisms, including but not limited to, Argonaute-mediated post-transcriptional cleavage of target gene mRNA transcripts (sometimes referred to in the art as RNAi) and/or other pre-transcriptional and/or pre-translational mechanisms.

RNA effector molecules disclosed herein include a RNA strand (the antisense strand) having a region which is 30 nucleotides or less in length, e.g., 10 to 30 nucleotides in length, or 19 to 24 nucleotides in length, which region is substantially complementary to at least a portion of a target gene that affects one or more aspects of the production of an immunogenic agent, such as the yield, purity, homogeneity, biological activity, or stability of the immunogenic agent. The RNA effector molecules interact with RNA transcripts of target genes and mediate their selective degradation or otherwise prevent their translation.

The term “antisense strand” refers to the strand of a RNA effector molecule, e.g., a dsRNA, which includes a region that is substantially complementary to a target sequence. The term “region of complementarity” refers to the region on the antisense strand that is substantially complementary to a sequence, for example a target sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′ and/or 3′ terminus.

The term “sense strand” refers to the strand of a RNA effector molecule that includes a region that is substantially complementary to a region of the antisense strand as that term is defined herein.

As used herein, and unless otherwise indicated, the term “complementary”, when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as understood by the skilled artisan. “Complementary” sequences can also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but are not limited to, G:U Wobble or Hoogstein base pairing. Hybridization conditions can, for example, be stringent conditions, where stringent conditions can include 400 mM NaCl, 40 mM PIPES pH 6.4, 1 mM EDTA, 50° C. or 70° C., for 12 to 16 hours followed by washing. Other conditions, such as physiologically relevant conditions as can be encountered inside an organism, can apply. The skilled artisan will be able to determine the set of conditions most appropriate for a test of complementarity of two sequences in accordance with the ultimate application of the hybridized nucleotides.

The terms “complementary,” “fully complementary” and “substantially complementary” herein can be used with respect to the base matching between the sense strand and the antisense strand of a dsRNA, or between the antisense strand of a RNA effector molecule agent and a target sequence, as will be understood from the context of use. As used herein, an oligonucleotide that is “substantially complementary to at least part of” a target gene refers to an oligonucleotide that is substantially complementary to a contiguous portion of a target gene of interest (e.g., a mRNA encoded by a target gene, the target gene's promoter region or 3′ UTR, or ERV LTR). For example, an oligonucleotide is complementary to at least a part of a target mRNA if the sequence is substantially complementary to a non-interrupted portion of an mRNA encoded by a target gene.

Complementary sequences within a RNA effector molecule, e.g., within a dsRNA (a double-stranded ribonucleic acid) as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. Where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. Where two oligonucleotides are designed to form, upon hybridization, one or more single-stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

In some embodiments, the RNA effector molecule comprises a single-stranded oligonucleotide that interacts with and directs the cleavage of RNA transcripts of a target gene. For example, single stranded RNA effector molecules comprise a 5′ modification including one or more phosphate groups or analogs thereof to protect the effector molecule from nuclease degradation. The RNA effector molecule can be a single-stranded antisense nucleic acid having a nucleotide sequence that is complementary to at least a portion of a “sense” nucleic acid of a target gene, e.g., the coding strand of a double-stranded cDNA molecule or a RNA sequence, e.g., a pre-mRNA, mRNA, miRNA, or pre-miRNA. Accordingly, an antisense nucleic acid can form hydrogen bonds with a sense nucleic acid target. In an alternative embodiment, the RNA effector molecule comprises a duplex region of at least nine nucleotides in length.

Given a coding strand sequence (e.g., the sequence of a sense strand of a cDNA molecule), antisense nucleic acids can be designed according to the rules of Watson-Crick base pairing. The antisense nucleic acid can be complementary to a portion of the coding or noncoding region of a RNA, e.g., the region surrounding the translation start site of a pre-mRNA or mRNA, e.g., the 5′ UTR. An antisense oligonucleotide can be, for example, about 10 to 25 nucleotides in length (e.g., 10, 11, 12, 13, 14, 15, 16, 18, 19, 20, 21, 22, 23, or 24 nucleotides in length). In some embodiments, the antisense oligonucleotide comprises one or more modified nucleotides, e.g., phosphorothioate derivatives and/or acridine substituted nucleotides, designed to increase its biological stability of the molecule and/or the physical stability of the duplexes formed between the antisense and target nucleic acids. Antisense oligonucleotides can comprise ribonucleotides only, deoxyribonucleotides only (e.g., oligodeoxynucleotides), or both deoxyribonucleotides and ribonucleotides. For example, an antisense agent consisting only of ribonucleotides can hybridize to a complementary RNA and prevent access of the translation machinery to the target RNA transcript, thereby preventing protein synthesis. An antisense molecule including only deoxyribonucleotides, or deoxyribonucleotides and ribonucleotides, can hybridize to a complementary RNA and the RNA target can be subsequently cleaved by an enzyme, e.g., RNAse H, to prevent translation. The flanking RNA sequences can include 2′-O-methylated nucleotides, and phosphorothioate linkages, and the internal DNA sequence can include phosphorothioate internucleotide linkages. The internal DNA sequence is preferably at least five nucleotides in length when targeting by RNAseH activity is desired.

In some embodiments, RNA effector molecule is a double-stranded oligonucleotide. The term “double-stranded RNA” or “dsRNA”, as used herein, refers to an oligonucleotide molecule or complex of molecules having a hybridized duplex region that comprises two anti-parallel and substantially complementary nucleic acid strands, which will be referred to as having “sense” and “antisense” orientations with respect to a target RNA. Typically, region of complementarity is 30 nucleotides or less in length, generally, for example, 10 to 26 nucleotides in length, 18 to 25 nucleotides in length, or 19 to 24 nucleotides in length, inclusive. Upon contact with a cell expressing the target gene, the RNA effector molecule inhibits the expression of the target gene by at least 10% as assayed by, for example, a PCR or branched DNA (bDNA)-based method, or by a protein-based method, such as by protein immunoblot. Expression of a target gene in cell culture can be assayed by measuring target gene mRNA levels, e.g., by bDNA or TAQMAN® assay, or by measuring protein levels, e.g., by immunofluorescence analysis.

The duplex region can be of any length that permits specific degradation of a desired target RNA through a RISC pathway, but will typically range from 9 to 36 base pairs in length, e.g., 15 to 30 base pairs in length. More specifically, the duplex region can be of any length that permits specific degradation of a desired target RNA through a RISC pathway, but will typically range from 9 to 36 base pairs in length, e.g., 15 to 30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range there between, including, but not limited to 15 to 30 base pairs, 15 to 26 base pairs, 15 to 23 base pairs, 15 to 22 base pairs, 15 to 21 base pairs, 15 to 20 base pairs, 15 to 19 base pairs, 15 to 18 base pairs, 15 to 17 base pairs, 18 to 30 base pairs, 18 to 26 base pairs, 18 to 23 base pairs, 18 to 22 base pairs, 18 to 21 base pairs, 18 to 20 base pairs, 19 to 30 base pairs, 19 to 26 base pairs, 19 to 23 base pairs, 19 to 22 base pairs, 19 to 21 base pairs, 19 to 20 base pairs, 20 to 30 base pairs, 20 to 26 base pairs, 20 to 25 base pairs, 20 to 24 base pairs, 20 to 23 base pairs, 20 to 22 base pairs, 20 to 21 base pairs, 21 to 30 base pairs, 21 to 26 base pairs, 21 to 25 base pairs, 21 to 24 base pairs, 21 to 23 base pairs, or 21 to 22 base pairs, inclusive.

dsRNAs generated in the cell by processing with Dicer and similar enzymes are generally in the range of 19 to 22 base pairs in length. One strand of the duplex region of a dsDNA comprises a sequence that is substantially complementary to a region of a target RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (a “hairpin loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of a dsRNA are comprised by separate RNA molecules, those molecules need not, but can be covalently connected. Where the two strands are connected covalently by means other than a hairpin loop, the connecting structure is referred to as a “linker.” The term “sRNA effector molecule” is also used herein to refer to a dsRNA.

Described herein are RNA effector molecules that modulate expression of a target gene. In one embodiment, the RNA effector molecule agent includes double-stranded ribonucleic acid (dsRNA) molecules for inhibiting the expression of a target gene in a cell, where the dsRNA includes an antisense strand having a region of complementarity which is complementary to at least a part of a target gene formed in the expression of a target gene, and where the region of complementarity is 30 nucleotides or less in length, generally 10 to 24 nucleotides in length, and where the dsRNA, upon contact with an cell expressing the target gene, inhibits the expression of the target gene by at least 10% as assayed by, for example, a PCR, PERT, or bDNA-based method, or by a protein-based method, such as a protein immunoblot (e.g., a western blot). Expression of a target gene in an cell can be assayed by measuring target gene mRNA levels, e.g., by PERT, bDNA or TAQMAN® gene expression assay, or by measuring protein levels, e.g., by immunofluorescence analysis or quantitative protein immunoblot.

A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence, derived, for example, from the sequence of an mRNA formed during the expression of a target gene. The other strand (the sense strand) includes a region that is complementary to the antisense strand, such that the two strands hybridize and form a duplex structure when combined under suitable conditions. Generally, the duplex structure is, for example between 9 and 36, between 10 to 30 base pairs, between 18 and 25, between 19 and 24, or between 19 and 21 base pairs in length, inclusive. Similarly, the region of complementarity to the target sequence is, for example, between 10 and 30, between 18 and 25, between 19 and 24, or between 19 and 21 nucleotides in length, inclusive. In some embodiments, the dsRNA is between 10 and 20 nucleotides in length, inclusive, and in other embodiments, the dsRNA is between 25 and 30 nucleotides in length, inclusive. Thus, in one embodiment, to the extent that it becomes processed to a functional duplex of e.g., 15 to 30 base pairs that targets a desired RNA for cleavage, a RNA molecule or complex of RNA molecules having a duplex region greater than 30 base pairs is a dsRNA. As the ordinarily skilled person will recognize, the targeted region of a RNA targeted for cleavage will most often be part of a larger RNA molecule, often a mRNA molecule.

Where relevant, a “part” of a mRNA target is a contiguous sequence of a mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 10 nucleotides in length, such as from 15 to 30 nucleotides in length, inclusive.

The skilled person is well aware that dsRNAs having a duplex structure of between 20 and 23, but specifically 21, base pairs have been hailed as particularly effective in inducing RNA interference. Elbashir et al., 20 EMBO 6877-88 (2001). In the embodiments described above, by virtue of the nature of the oligonucleotide sequences, dsRNAs described herein can include at least one strand of a length of 21 nucleotides. It can be reasonably expected that shorter duplexes having one of the sequences minus only a few nucleotides on one or both ends can be similarly effective as compared to the dsRNAs described in detail. Hence, dsRNAs having a partial sequence of at least 10, 11, 12, 13, 14, 15, 16, 17, 18, 19, 20, or more contiguous nucleotides from a given sequence, and differing in their ability to inhibit the expression of a target gene by not more than 5%, 10%, 15%, 20%, 25%, or 30% inhibition from a dsRNA comprising the full sequence, are contemplated according to the invention.

The dsRNA can be synthesized by standard methods known in the art as further discussed below, e.g., by use of an automated DNA synthesizer, such as are commercially available from, for example, Biosearch Technologies (Novato, Calif.). In one embodiment, a target gene is a human target gene. In specific embodiments, the first sequence is a sense strand of a dsRNA that includes a sense sequence and the second sequence is a strand of a ds RNA that includes an antisense sequence. Alternative dsRNA agents that target elsewhere in the target sequence can readily be determined using the target sequence and the flanking target sequence. In this aspect, one of the two sequences is complementary to the other of the two sequences, with one of the sequences being substantially complementary to a sequence of an mRNA generated in the expression of a target gene. As such, in this aspect, a dsRNA will include two oligonucleotides, where one oligonucleotide is described as the sense strand and the second oligonucleotide is described as the antisense strand. As described elsewhere herein and as known in the art, the complementary sequences of a dsRNA can also be contained as self-complementary regions of a single nucleic acid molecule, as opposed to being on separate oligonucleotides.

A double-stranded oligonucleotide can include one or more single-stranded nucleotide overhangs. As used herein, the term “nucleotide overhang” refers to at least one unpaired nucleotide that protrudes from the terminus of a duplex structure of a double-stranded oligonucleotide, e.g., a dsRNA. For example, when a 3′-end of one strand of double-stranded oligonucleotide extends beyond the 5′-end of the other strand, or vice versa, there is a nucleotide overhang. A double-stranded oligonucleotide can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog. The overhang(s) can be on the sense strand, the antisense strand or any combination thereof. Furthermore, the nucleotide(s) of an overhang can be present on the 5′ end, 3′ end, or both ends of either an antisense or sense strand of a dsRNA.

In one embodiment, at least one end of a dsRNA has a single-stranded nucleotide overhang of 1 to 4, generally 1 or 2 nucleotides. dsRNAs having at least one nucleotide overhang have unexpectedly superior inhibitory properties relative to their blunt-ended counterparts. Moreover, the presence of a nucleotide overhang on only one strand, at one end of a dsRNA, strengthens the interference activity of the dsRNA, without affecting its overall stability. Such an overhang need not be a single nucleotide overhang; a dinucleotide overhang can also be present.

The antisense strand of a double-stranded oligonucleotide has a 1 to 10 nucleotide overhang at the 3′ end and/or the 5′ end, such as a double-stranded oligonucleotide having a 1 to 10 nucleotide overhang at the 3′ end and/or the 5′ end. One or more of the internucleoside linkages in the overhang can be replaced with a phosphorothioate. In some embodiments, the overhang comprises one or more deoxyribonucleoside or the overhang comprises one or more dT, e.g. the sequence 5′-dTdT-3′ or 5′-dTdTdT-3′. In some embodiments, overhang comprises the sequence 5′-dT*dT-3, wherein * is a phosphorothioate internucleoside linkage.

Without being bound theory, double-stranded oligonucleotides having at least one nucleotide overhang have unexpectedly superior inhibitory properties relative to their blunt-ended counterparts. Moreover, the presence of a nucleotide overhang on only one strand, at one end of a dsRNA, strengthens the interference activity of the double-stranded oligonucleotide, without affecting its overall stability.

dsRNA having only one overhang has proven particularly stable and effective in vivo, as well as in a variety of cells, cell culture media, blood, and serum. Generally, the single-stranded overhang is located at the 3′-terminal end of an antisense strand or, alternatively, at the 3′-terminal end of a sense strand. The dsRNA having an overhang on only one end will also have one blunt end, generally located at the 5′-end of the antisense strand. Such dsRNAs have superior stability and inhibitory activity, thus allowing administration at low dosages, i.e., less than 5 mg/kg body weight of the recipient per day. In one embodiment, the antisense strand of a dsRNA has a 1 to 10 nucleotide overhang at the 3′ end and/or the 5′ end. In one embodiment, the sense strand of a dsRNA has a 1 to 10 nucleotide overhang at the 3′ end and/or the 5′ end. In another embodiment, one or more of the nucleotides in the overhang is replaced with a nucleoside thiophosphate.

The terms “blunt” or “blunt ended” as used herein in reference to double-stranded oligonucleotide mean that there are no unpaired nucleotides or nucleotide analogs at a given terminal end of a double-stranded oligonucleotide, i.e., no nucleotide overhang. One or both ends of a double-stranded oligonucleotide can be blunt. Where both ends are blunt, the oligonucleotide is said to be double-blunt ended. To be clear, a “double-blunt ended” oligonucleotide is a double-stranded oligonucleotide that is blunt at both ends, i.e., no nucleotide overhang at either end of the molecule. Most often such a molecule will be double-stranded over its entire length. When only one end of is blunt, the oligonucleotide is said to be single-blunt ended. To be clear, a “single-blunt ended” oligonucleotide is a double-stranded oligonucleotide that is blunt at only one end, i.e., no nucleotide overhang at one end of the molecule. Generally, a single-blunt ended oligonucleotide is blunt ended at the 5′-end of sense stand.

A RNA effector molecule as described herein can contain one or more mismatches to the target sequence. For example, a RNA effector molecule as described herein contains no more than three mismatches. If the antisense strand of the RNA effector molecule contains mismatches to a target sequence, it is preferable that the area of mismatch not be located in the center of the region of complementarity. If the antisense strand of the RNA effector molecule contains mismatches to the target sequence, it is preferable that the mismatch be restricted to be within the last 5 nucleotides from either the 5′ or 3′ end of the region of complementarity. For example, for a 23-nucleotide RNA effector molecule agent RNA strand which is complementary to a region of a target gene, the RNA strand generally does not contain any mismatch within the central 13 nucleotides. The methods described herein, or methods known in the art, can be used to determine whether a RNA effector molecule containing a mismatch to a target sequence is effective in inhibiting the expression of a target gene. Consideration of the efficacy of RNA effector molecules with mismatches in inhibiting expression of a target gene is important, especially if the particular region of complementarity in a target gene is known to have polymorphic sequence variation within the population.

In some embodiments, the RNA effector molecule is a promoter-directed RNA (pdRNA) which is substantially complementary to at least a portion of a noncoding region of an mRNA transcript of a target gene. In one embodiment, the pdRNA is substantially complementary to at least a portion of the promoter region of a target gene mRNA at a site located upstream from the transcription start site, e.g., more than 100, more than 200, or more than 1,000 bases upstream from the transcription start site. In another embodiment, the pdRNA is substantially complementary to at least a portion of the 3′-UTR of a target gene mRNA transcript. In one embodiment, the pdRNA comprises dsRNA of 18-28 bases optionally having 3′ di- or tri-nucleotide overhangs on each strand. The dsRNA is substantially complementary to at least a portion of the promoter region or the 3′-UTR region of a target gene mRNA transcript. In another embodiment, the pdRNA comprises a gapmer consisting of a single stranded polynucleotide comprising a DNA sequence which is substantially complementary to at least a portion of the promoter or the 3′-UTR of a target gene mRNA transcript, and flanking the polynucleotide sequences (e.g., comprising the 5 terminal bases at each of the 5′ and 3′ ends of the gapmer) comprising one or more modified nucleotides, such as 2′ MOE, 2′OMe, or Locked Nucleic Acid bases (LNA), which protect the gapmer from cellular nucleases.

pdRNA can be used to selectively increase, decrease, or otherwise modulate expression of a target gene. Without being limited to theory, it is believed that pdRNAs modulate expression of target genes by binding to endogenous antisense RNA transcripts which overlap with noncoding regions of a target gene mRNA transcript, and recruiting Argonaute proteins (in the case of dsRNA) or host cell nucleases (e.g., RNase H) (in the case of gapmers) to selectively degrade the endogenous antisense RNAs. In some embodiments, the endogenous antisense RNA negatively regulates expression of the target gene and the pdRNA effector molecule activates expression of the target gene. Thus, in some embodiments, pdRNAs can be used to selectively activate the expression of a target gene by inhibiting the negative regulation of target gene expression by endogenous antisense RNA. Methods for identifying antisense transcripts encoded by promoter sequences of target genes and for making and using promoter-directed RNAs are known, see, e.g., WO 2009/046397.

In some embodiments, the RNA effector molecule comprises an aptamer which binds to a non-nucleic acid ligand, such as a small organic molecule or protein, e.g., a transcription or translation factor, and subsequently modifies (e.g., inhibits) activity. An aptamer can fold into a specific structure that directs the recognition of a targeted binding site on the non-nucleic acid ligand. Aptamers can contain any of the modifications described herein.

In some embodiments, the RNA effector molecule comprises an antagomir. Antagomirs are single stranded, double stranded, partially double stranded or hairpin structures that target a microRNA. An antagomir consists essentially of or comprises at least 10 or more contiguous nucleotides substantially complementary to an endogenous miRNA and more particularly a target sequence of an miRNA or pre-miRNA nucleotide sequence. Antagomirs preferably have a nucleotide sequence sufficiently complementary to a miRNA target sequence of about 12 to 25 nucleotides, such as about 15 to 23 nucleotides, to allow the antagomir to hybridize to the target sequence. More preferably, the target sequence differs by no more than 1, 2, or 3 nucleotides from the sequence of the antagomir. In some embodiments, the antagomir includes a non-nucleotide moiety, e.g., a cholesterol moiety, which can be attached, e.g., to the 3′ or 5′ end of the oligonucleotide agent.

In some embodiments, antagomirs are stabilized against nucleolytic degradation by the incorporation of a modification, e.g., a nucleotide modification. For example, in some embodiments, antagomirs contain a phosphorothioate comprising at least the first, second, and/or third internucleotide linkages at the 5′ or 3′ end of the nucleotide sequence. In further embodiments, antagomirs include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O -dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In some embodiments, antagomirs include at least one 2′-O-methyl-modified nucleotide.

In some embodiments, the RNA effector molecule is a promoter-directed RNA (pdRNA) which is substantially complementary to at least a portion of a noncoding region of an mRNA transcript of a target gene. The pdRNA can be substantially complementary to at least a portion of the promoter region of a target gene mRNA at a site located upstream from the transcription start site, e.g., more than 100, more than 200, or more than 1,000 bases upstream from the transcription start site. Also, the pdRNA can substantially complementary to at least a portion of the 3′-UTR of a target gene mRNA transcript. For example, the pdRNA comprises dsRNA of 18 to 28 bases optionally having 3′ di- or tri-nucleotide overhangs on each strand. The dsRNA is substantially complementary to at least a portion of the promoter region or the 3′-UTR region of a target gene mRNA transcript. In another embodiment, the pdRNA comprises a gapmer consisting of a single stranded polynucleotide comprising a DNA sequence which is substantially complementary to at least a portion of the promoter or the 3′-UTR of a target gene mRNA transcript, and flanking the polynucleotide sequences (e.g., comprising the 5 terminal bases at each of the 5′ and 3′ ends of the gapmer) comprising one or more modified nucleotides, such as 2′MOE, 2′OMe, or Locked Nucleic Acid bases (LNA), which protect the gapmer from cellular nucleases.

pdRNA can be used to selectively increase, decrease, or otherwise modulate expression of a target gene. Without being limited to theory, pdRNAs can modulate expression of target genes by binding to endogenous antisense RNA transcripts which overlap with noncoding regions of a target gene mRNA transcript, and recruiting Argonaute proteins (in the case of dsRNA) or host cell nucleases (e.g., RNase H) (in the case of gapmers) to selectively degrade the endogenous antisense RNAs. In some embodiments, the endogenous antisense RNA negatively regulates expression of the target gene and the pdRNA effector molecule activates expression of the target gene. Thus, in some embodiments, pdRNAs can be used to selectively activate the expression of a target gene by inhibiting the negative regulation of target gene expression by endogenous antisense RNA. Methods for identifying antisense transcripts encoded by promoter sequences of target genes and for making and using promoter-directed RNAs are known. See, e.g., WO 2009/046397.

Expressed interfering RNA (eiRNA) can be used to selectively increase, decrease, or otherwise modulate expression of a target gene. Typically, eiRNA, the dsRNA is expressed in the first transfected cell from an expression vector. In such a vector, the sense strand and the antisense strand of the dsRNA can be transcribed from the same nucleic acid sequence using e.g., two convergent promoters at either end of the nucleic acid sequence or separate promoters transcribing either a sense or antisense sequence. Alternatively, two plasmids can be cotransfected, with one of the plasmids designed to transcribe one strand of the dsRNA while the other is designed to transcribe the other strand. Methods for making and using eiRNA effector molecules are known in the art. See, e.g., WO 2006/033756; U.S. Patent Pubs. No. 2005/0239728 and No. 2006/0035344.

In some embodiments, the RNA effector molecule comprises a small single-stranded Piwi-interacting RNA (piRNA effector molecule) which is substantially complementary to at least a portion of a target gene, as defined herein, and which selectively binds to proteins of the Piwi or Aubergine subclasses of Argonaute proteins. Without being limited to a particular theory, it is believed that piRNA effector molecules interact with RNA transcripts of target genes and recruit Piwi and/or Aubergine proteins to form a ribonucleoprotein (RNP) complex that induces transcriptional and/or post-transcriptional gene silencing of target genes. A piRNA effector molecule can be about 10 to 50 nucleotides in length, about 25 to 39 nucleotides in length, or about 26 to 31 nucleotides in length. See, e.g., U.S. Patent Pub. No. 2009/0062228.

MicroRNAs are a highly conserved class of small RNA molecules that are transcribed from DNA in the genomes of plants and animals, but are not translated into protein. Pre-microRNAs are processed into miRNAs. Processed microRNAs are single stranded ˜17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. MicroRNAs cause post-transcriptional silencing of specific target genes, e.g., by inhibiting translation or initiating degradation of the targeted mRNA. In some embodiments, the miRNA is completely complementary with the target nucleic acid. In other embodiments, the miRNA has a region of noncomplementarity with the target nucleic acid, resulting in a “bulge” at the region of non-complementarity. In some embodiments, the region of noncomplementarity (the bulge) is flanked by regions of sufficient complementarity, e.g., complete complementarity, to allow duplex formation. For example, the regions of complementarity are at least 8 to 10 nucleotides long (e.g., 8, 9, or 10 nucleotides long).

miRNA can inhibit gene expression by, e.g., repressing translation, such as when the miRNA is not completely complementary to the target nucleic acid, or by causing target RNA degradation, when the miRNA binds its target with perfect or a high degree of complementarity. In further embodiments, the RNA effector molecule can include an oligonucleotide agent which targets an endogenous miRNA or pre-miRNA. For example, the RNA effector can target an endogenous miRNA which negatively regulates expression of a target gene, such that the RNA effector alleviates miRNA-based inhibition of the target gene. The oligonucleotide agent can include naturally occurring nucleobases, sugars, and covalent internucleotide (backbone) linkages and/or oligonucleotides having one or more non-naturally-occurring features that confer desirable properties, such as enhanced cellular uptake, enhanced affinity for the endogenous miRNA target, and/or increased stability in the presence of nucleases. In some embodiments, an oligonucleotide agent designed to bind to a specific endogenous miRNA has substantial complementarity, e.g., at least 70%, 80%, 90%, or 100% complementary, with at least 10, 20, or 25 or more bases of the target miRNA. Exemplary oligonucleotide agents that target miRNAs and pre-miRNAs are described, for example, in U.S. Patent Pubs. No. 20090317907, No. 20090298174, No. 20090291907, No. 20090291906, No. 20090286969, No. 20090236225, No. 20090221685, No. 20090203893, No. 20070049547, No. 20050261218, No. 20090275729, No. 20090043082, No. 20070287179, No. 20060212950, No. 20060166910, No. 20050227934, No. 20050222067, No. 20050221490, No. 20050221293, No. 20050182005, and No. 20050059005.

A miRNA or pre-miRNA can be 10 to 200 nucleotides in length, for example from 16 to 80 nucleotides in length. Mature miRNAs can have a length of 16 to 30 nucleotides, such as 21 to 25 nucleotides, particularly 21, 22, 23, 24, or 25 nucleotides in length. miRNA precursors can have a length of 70 to 100 nucleotides and can have a hairpin conformation. In some embodiments, miRNAs are generated in vivo from pre-miRNAs by the enzymes cDicer and Drosha. miRNAs or pre-miRNAs can be synthesized in vivo by a cell-based system or can be chemically synthesized. miRNAs can comprise modifications which impart one or more desired properties, such as superior stability, hybridization thermodynamics with a target nucleic acid, targeting to a particular tissue or cell-type, and/or cell permeability, e.g., by an endocytosis-dependent or -independent mechanism. Modifications can also increase sequence specificity, and consequently decrease off-site targeting.

In further embodiments, the RNA effector molecule can comprise an oligonucleotide agent which targets an endogenous miRNA or pre-miRNA. For example, the RNA effector can target an endogenous miRNA which negatively regulates expression of a target gene, such that the RNA effector alleviates miRNA-based inhibition of the target gene.

As used herein, the phrase “in the presence of at least one RNA effector molecule” encompasses exposure of the cell to a RNA effector molecule expressed within the cell, e.g., shRNA, or exposure by exogenous addition of the RNA effector molecule to the cell, e.g., delivery of the RNA effector molecule to the cell, optionally using an agent that facilitates uptake into the cell. A portion of a RNA effector molecule is substantially complementary to at least a portion of the target gene RNA, such as the coding region, the promoter region, the 3′untranslated region (3′-UTR), or a long terminal repeat (LTR) of the target gene RNA. RNA effector molecules disclosed herein include a RNA strand (the antisense strand) having a region which is 30 nucleotides or less in length, e.g., 10 to 200 nucleotides in length, or 19 to 24 nucleotides in length, which region is substantially complementary to at least a portion of a target gene which encodes a protein that affects one or more aspects of the production of a immunogenic agent, such as the yield, purity, homogeneity, biological activity, or stability of the immunogenic agent. A RNA effector molecule interacts with RNA transcripts of a target gene and mediates its selective degradation or otherwise prevents its translation. In various embodiments of the present invention, the RNA effector molecule is at least one gapmer, or siRNA, miRNA, dsRNA, saRNA, shRNA, piRNA, tkRNAi, eiRNA, pdRNA, antagomir, or ribozyme.

Double-stranded and single-stranded oligonucleotides that are effective in inducing RNA interference are also referred to as siRNA, RNAi agent, or iRNA agent, herein. These RNA interference inducing oligonucleotides associate with a cytoplasmic multi-protein complex known as RNAi-induced silencing complex (RISC). Without being bound by theory, RNA interference leads to Argonaute-mediated post-transcriptional cleavage of target gene mRNA transcripts. In many embodiments, single-stranded and double-stranded RNAi agents are sufficiently long that they can be cleaved by an endogenous molecule, e.g. by Dicer, to produce smaller oligonucleotides that can enter the RISC machinery and participate in RISC mediated cleavage of a target sequence, e.g., a target mRNA.

In some embodiments, the RNAs provided herein identify a site in a target transcript that is susceptible to RISC-mediated cleavage. As such, the present invention further features RNA effector molecules that target within one of such sequences. Such a RNA effector molecule will generally include at least 10 contiguous nucleotides from one of the sequences provided coupled to additional nucleotide sequences taken from the region contiguous to the selected sequence in a target gene.

The phrase “genome information” as used herein and throughout the claims and specification is meant to refer to sequence information from partial or entire genome of an organism, including protein coding and non-coding regions. These sequences are present every cell originating from the same organisms. As opposed to the transcriptome sequence information, genome information comprises not only coding regions, but also, for example, intronic sequences, promoter sequences, silencer sequences and enhancer sequences. Thus, the “genome information” can refer to, for example a human genome, a mouse genome, a rat genome. One can use complete genome information or partial genome information to add an additional dimension to the database sequences to increase the potential targets to modify with a RNA effector molecule.

The phrase “play a role” refers to any activity of a transcript or a protein in a molecular pathway known to a skilled artisan or identified elsewhere in this specification. Such pathways an cellular activities include, but are not limited to apoptosis, cell division, glycosylation, growth rate, a cellular productivity, a peak cell density, a sustained cell viability, a rate of ammonia production or consumption, or a rate of lactate production.

A “bioreactor”, as used herein, refers generally to any reaction vessel suitable for growing and maintaining host cells such that the host cells produce an immunogenic agent, and for recovering such immunogenic agent. Bioreactors described herein include cell culture systems of varying sizes, such as small culture flasks, Nunc multilayer cell factories, small high yield bioreactors (e.g., MiniPerm, INTEGRA-CELLine), spinner flasks, hollow fiber-WAVE bags (Wave Biotech, Tagelswangen, Switzerland), and industrial scale bioreactors. In some embodiments, the immunogenic agent is produced in a “large scale culture” bioreactor having a 1 L capacity or more, suitable for pharmaceutical or industrial scale production of immunogenic agents (e.g., a volume of at least 1 L, least 2 L, at least 5 L, at least 10 L, at least 25 L, at least 50 L, at least 100 L, or more, inclusive), often including means of monitoring pH, glucose, lactate, temperature, and/or other bioprocess parameters. In one embodiment, a large scale culture is at least 1 L in volume.

In one embodiment, a large scale culture is at least 2 L in volume. In one embodiment, a large scale culture is at least 5 L in volume. In one embodiment, a large scale culture is at least 25 L in volume. In one embodiment, a large scale culture is at least 40 L in volume. In one embodiment, a large scale culture is at least 50 L in volume. In one embodiment, a large scale culture is at least 100 L in volume.

A “host cell”, as used herein, is any cell, cell culture, cellular biomass or tissue, capable of being grown and maintained in cell culture under conditions allowing for production and recovery of useful quantities of an immunogenic agent, as defined herein. A host cell can be derived from a yeast, insect, amphibian, fish, reptile, bird, mammal or human, or can be a hybridoma cell. Host cells can be unmodified cells or cell lines, or cell lines which have been genetically modified (e.g., to facilitate production of an immunogenic agent). In some embodiments, the host cell is a cell line that has been modified to allow for growth under desired conditions, such as in serum-free media, in cell suspension culture, or in adherent cell culture. As used herein, “hamster” refers to Cricetulus griseus (Chinese hamster).

A mammalian host cell can be advantageous where the immunogenic agent is a mammalian recombinant polypeptide, particularly if the polypeptide is a biotherapeutic agent or is otherwise intended for administration to or consumption by humans. In some embodiments, the host cell is a CHO cell, which is a cell line used for the expression of many recombinant proteins. Additional mammalian cell lines used commonly for the expression of recombinant proteins include 293HEK cells, HeLa cells, COS cells, NIH/3T3 cells, Jurkat Cells, NSO cells. and HUVEC cells.

In one embodiment, the host cell is a Madin Darby canine kidney (MDCK) cell. MDCK cells are routinely used by those of skill in the art for virus/vaccine production.

In some embodiments, the host cell is a CHO cell derivative that has been modified genetically to facilitate production of recombinant proteins or other immunogenic agents. For example, various CHO cell strains have been developed which permit stable insertion of recombinant DNA into a specific gene or expression region of the cells, amplification of the inserted DNA, and selection of cells exhibiting high level expression of the recombinant protein. Examples of CHO cell derivatives useful in methods provided herein include, but are not limited to, CHO-K1 cells, CHO-DUKX, CHO-DUKX B1, CHO-DG44 cells, CHO—ICAM-1 cells, and CHO-h1FNγ cells. Methods for expressing recombinant proteins in CHO cells are known in the art and are described in, e.g., U.S. Pat. No. 4,816,567 and No. 5,981,214.

Examples of human cell lines useful in methods provided herein include the cell lines 293T. (embryonic kidney), 786-0 (renal), A498 (renal), A549 (alveolar basal epithelial), ACHN (renal), BT-549 (breast), BxPC-3 (pancreatic), CAKI-1 (renal), Capan-1 (pancreatic), CCRF-CEM (leukemia), COLO 205 (colon), DLD-1 (colon), DMS 114 (small cell lung), DU145 (prostate), EKVX (non-small cell lung), HCC-2998 (colon), HCT-15 (colon), HCT-116 (colon), HT29 (colon), HT-1080 (fibrosarcoma), HEK 293 (embryonic kidney), HeLa (cervical carcinoma), HepG2 (hepatocellular carcinoma), HL-60(TB) (leukemia), HOP-62 (non-small cell lung), HOP-92 (non-small cell lung), HS 578T. (breast), HT-29 (colon adenocarcinoma), IGR-OV1 (ovarian), IMR32 (neuroblastoma), Jurkat (T lymphocyte), K-562 (leukemia), KM12 (colon), KM20L2 (colon), LANS (neuroblastoma), LNCap.FGC (Caucasian prostate adenocarcinoma), LOX IMVI (melanoma), LXFL 529 (non-small cell lung), M14 (melanoma), M19-MEL (melanoma), MALME-3M (melanoma), MCFlOA (mammary epithelial), MCF7 (mammary), MDA-MB-453 (mammary epithelial), MDA-MB-468 (breast), MDA-MB-231 (breast), MDA-N (breast), MOLT-4 (leukemia), NCI/ADR-RES (ovarian), NCI-H226 (non-small cell lung), NCI—H23 (non-small cell lung), NCI—H322M (non-small cell lung), NCI—H460 (non-small cell lung), NCI—H522 (non-small cell lung), OVCAR-3 (ovarian), OVCAR-4 (ovarian), OVCAR-5 (ovarian), OVCAR-8 (ovarian), P388 (leukemia), P388/ADR (leukemia), PC-3 (prostate), PERC6® (E1-transformed embryonal retina), RPMI-7951 (melanoma), RPMI-8226 (leukemia), RXF 393 (renal), RXF-631 (renal), Saos-2 (bone), SF-268 (CNS), SF-295 (CNS), SF-539 (CNS), SHP-77 (small cell lung), SH-SY5Y (neuroblastoma), SK-OV-3 (breast), SK-MEL-2 (melanoma), SK-MEL-5 (melanoma), SK-MEL-28 (melanoma), SK-OV-3 (ovarian), SN12K1 (renal), SN12C (renal), SNB-19 (CNS), SNB-75 (CNS)SNB-78 (CNS), SR (leukemia), SW-620 (colon), T-47D (breast), THP-1 (monocyte-derived macrophages), TK-10 (renal), U87 (glioblastoma), U293 (kidney), U251 (CNS), UACC-257 (melanoma), UACC-62 (melanoma), UO-31 (renal), W138 (lung), and XF 498 (CNS).

Examples of non-human primate cell lines useful in methods provided herein include the cell lines monkey kidney (CVI-76), African green monkey kidney (VERO-76), green monkey fibroblast (COS-1), and monkey kidney (CVI) cells transformed by SV40 (COS-7). Additional mammalian cell lines are known to those of ordinary skill in the art and are catalogued at the American Type Culture Collection catalog (Manassas, Va.).

Additional examples of rodent cell lines useful in methods provided herein include the cell lines baby hamster kidney (BHK) (e.g., BHK21, BHK TK), mouse Sertoli (TM4), buffalo rat liver (BRL 3A), mouse mammary tumor (MMT), rat hepatoma (HTC), mouse myeloma (NS0), murine hybridoma (Sp2/0), mouse thymoma (EL4), murine embryonic (NIH/3T3, 3T3 Ll), rat myocardial (H9c2), mouse myoblast (C2C12), and mouse kidney (miMCD-3).

In some embodiments, the host cell is a multipotent stem cell or progenitor cell. Examples of multipotent cells useful in methods provided herein include murine embryonic stem (ES-D3) cells, human umbilical vein endothelial (HuVEC) cells, human umbilical artery smooth muscle (HuASMC) cells, human differentiated stem (HKB-II) cells, human mesenchymal stem (hMSC) cells, and induced pluripotent stem (iPS) cells.

In some embodiments, the host cell is a plant cell. Examples of plant cells that grow readily in culture include Arabidopsis thaliana (cress), Allium sativum (garlic) Taxus chinensis, T. cuspidata, T. baccata, T. brevifolia and T. mairei (yew), Catharanthus roseus (periwinkle), Nicotiana benthamiana (solanaceae), N. tabacum (tobacco) including tobacco cells lines such as NT-1 or BY-2 (NT-1 cells are available from ATCC, No. 74840, see also U.S. Pat. No. 6,140,075), Oryza sativa (rice), Lycopersicum esulentum (tomato), Medicago sativa (alfalfa), Glycine max (soybean), Medicago truncatula and M. sativa (clovers), Phaseolus vulgaris (bean), Solanum tuberosum (potato), Beta vulgaris (beet), Saccharum spp. (sugarcane), Tectona grandis (teak), Musa spp. (banana), Phyllostachys nigra (bamboo), Vitis vinifera and V. gamay (grape), Popuius alba (poplar), Elaeis guineensis (oil palm), Ulmus spp. (elm), Thalictrum minus (meadow rue), Tinospora cordifolia ( ), Vinca rosea (vinca), Sorghum spp., Lolium perenne (ryegrass), Cucumis sativus (cucumber), Asparagus officinalis, Brucea javanica (Yadanxi), Doritaenopsis and Phalaenopsis (orchids), Rubus chamaemorus (cloudberry), Coffea arabica, Triticum timopheevii (wheat), Actinidia deliciosa (kiwi), Typha latifolia (cattail), Azadirachta indica (neem), Uncaria tomentosa and U. guianensis (cat's claw), Platycodon grandiflorum (balloon flower), Calotropis gigantea (mikweed), Kosteletzkya virginica (mallow), Pyrus malus (apple), Papaver somniferum (opium poppy), Citrus ssp., Choisya ternata (mock orange), Galium mollugo (madder), Digitalis Janata and D. purpurea (foxglove), Stevia rebaudiana (sweetleaf), Stizolobium hassjoo (purselane), Panicum virgatum (switchgrass), Rudgea jasminoides, Panax quinquefolius (American ginseng), Cupressus macrocarpa and C. arizonica (cypress), Vetiveria zizanioides (vetiver grass), Withania somnifera (Indian ginseng), Vigna unguiculata (cowpea), Phyllanthus niruri (spurge), Pueraria tuberosa and P. lobata (kudzu), Glycyrrhiza echinata (liquorice), Cicer arietinum (chick pea), Silybum marianum (milk thistle), Callistemon citrinus (bottle brush tree), Astragalus chrysochlorus (cuckoo flower), Coronilla vaginalis, such as cell line 39 RAR (crown vetch), Salvia miltiorrhiza (red sage), Vigna radiata (mung bean), Gisekia pharmaceoides, Datura tatula and D. stramonium (devil's trumpet), and Zea mays spp. (maize/corn).

The plant cell cultures provided herein are not limited to any particular method for transforming plant cells. Technology for introducing DNA into plant cells is well-known to those of skill in the art. See, e.g., U.S. Patent Application Pub. No. 2010/0009449. Basic methods for delivering foreign DNA into plant cells have been described, including chemical methods (Graham & van der Eb, 54 Virol. 536-39 (1973); Zatloukal et al., 660 Ann. NY Acad. Sci. 136-53 (1992)); physical methods, including microinjection (Capeechi, 22 Cell 479-88 (1980), electroporation (Wong & Neumann, 107 Biochem. Biophys. Res. Commn. 584-87 (1982); Fromm et al., 82 PNAS 5824-28 (1985); U.S. Pat. No. 5,384,253), and the “gene gun” (Johnston & Tang, 43 Met. Cell. Biol. 353-65 (1994); Fynan et al., 90 PNAS 11478-82 (1993)); viral methods (Clapp, 20 Clin. Perinatol. 155-68 (1993); Lu et al., 178 J. Exp. Med. 2089-96 (1993); Eglitis & Anderson, 6 Biotechs. 608-14 (1988); Eglitis et al., 241 Avd. Exp. Med. Biol. 19-27 (1988); and receptor-mediated methods (Curiel et al., 88 PNAS 8850-54 (1991); Curiel et al., 3 Hum. Gen. Ther. 147-54 (1992); Wagner et al., 89 PNAS 6099-103 (1992). Transgenic plant is herein defined as a plant cell culture, plant cell line, plant tissue culture, lower plant, monocot plant cell culture, dicot plant cell culture, or progeny thereof derived from a transformed plant cell or protoplast, wherein the genome of the transformed plant contains foreign DNA, introduced by laboratory techniques, not originally present in a native, non-transgenic plant cell of the same species.

In some embodiments, the host cell is fungal, such as Sacharomyces cerevisiae, Pichia pastoris or P. methanolica, Rhizopus, Aspergillus, Scizosacchromyces pombe, Hansanuela polymorpha, or Kluyveromyces lactis. See, e.g., Petranovic & Vemuri, 144 J. Biotech. 204-11 (2009); Bollok et al., 3 Recent Pat. Biotech. 192-201 (2009); Takegawa et al., 53 Biotech. Appl. Biochem. 227-35 (2009); Chiba & Akeboshi, 32 Biol. Pharm. Bull. 786-95 (2009).

In some embodiments, the host cell is an insect cell, such as Sf9 cell line (derived from pupal ovarian tissue of Spodoptera frugiperda); Hi-5 (derived from Trichoplusia ni egg cell homogenates); or S2 cells (from Drosophila melanogaster).

In some embodiments, the host cells are suitable for growth in suspension cultures. Suspension-competent host cells are generally monodisperse or grow in loose aggregates without substantial aggregation. Suspension-competent host cells include cells that are suitable for suspension culture without adaptation or manipulation (e.g., hematopoietic cells, lymphoid cells) and cells that have been made suspension-competent by modification or adaptation of attachment-dependent cells (e.g., epithelial cells, fibroblasts).

In some embodiments, the host cell is an attachment dependent cell which is grown and maintained in adherent culture. Examples of human adherent cell lines useful in methods provided herein include the cell lines human neuroblastoma (SH-SY5Y, IMR32, and LANS), human cervical carcinoma (HeLa), human breast epithelial (MCFlOA), human embryonic kidney (293T), and human breast carcinoma (SK-BR3).

In some embodiments, the host cell is a cell line that has been modified to allow for growth under desired conditions, such as in serum-free media, in cell suspension culture, or in adherent cell culture. The host cell can be, for example, a human Namalwa Burkitt lymphoma cell (BLcl-kar-Namalwa), baby hamster kidney fibroblast (BHK), CHO cell, Murine myeloma cell (NS0, SP2/0), hybridoma cell, human embryonic kidney cell (293 HEK), human retina-derived cell (PER.C6® cells, U.S. Pat. No. 7,550,284), insect cell line (Sf9, derived from pupal ovarian tissue of Spodoptera frugiperda; or Hi-5, derived from Trichoplusia ni egg cell homogenates; see also U.S. Pat. No. 7,041,500), Madin-Darby canine kidney cell (MDCK), primary mouse brain cells or tissue, primary calf lymph cells or tissue, primary monkey kidney cells, embryonated hens' egg, primary chicken embryo fibroblast (CEF), Rhesus fetal lung cell (FRhL-2), Human fetal lung cell (WI-38, MRC-5), African green monkey kidney epithelial cell (Vero, CV-1), Rhesus monkey kidney cell (LLC-MK2), or yeast cell. Additional mammalian cell lines commonly used for the expression of recombinant proteins include, but are not limited to, HeLa cells, COS cells, NIH/3T3 cells, Jurkat Cells, and human umbilical vein endothelial cells (HUVEC) cells.

Host cells can be unmodified or genetically modified (e.g., a cell from a transgenic animal). For example, CEFs from transgenic chicken eggs can have one or more genes essential for the IFN pathway, e.g., interferon receptor, STAT1, etc., disrupted, i.e., a trangenic “knockout.” See, e.g., Sang, 12 Trends Biotech. 415 (1994); Perry et al., 2 Transgenic Res. 125 (1993); Stern, 212 Curr Top Micro. Immunol. 195-206 (1996); Shuman, 47 Experientia 897 (1991). Also, the cell can be modified to allow for growth under desired conditions, e.g., incubation at 30° C.

In some embodiments, the host cells are suitable for growth in suspension cultures. Suspension-competent host cells are generally monodisperse or grow in loose aggregates without substantial aggregation. Suspension-competent host cells include cells that are suitable for suspension culture without adaptation or manipulation (e.g., hematopoietic cells, lymphoid cells) and cells that have been made suspension-competent by modification or adaptation of attachment-dependent cells (e.g., epithelial cells, fibroblasts). In some embodiments, the host cell is an attachment dependent cell which is grown and maintained in adherent culture. In some embodiments, the host cell is contained in an egg, such as a fish, amphibian, or avian egg.

“Isolating immunogenic agent from the host cell” means at least one step in separating the immunogenic agent away from host cellular material, e.g., the host cell, host cell culture medium, host cellular biomass, or host tissue. Thus, isolating immunogenic agents that are secreted into, and ultimately harvested from, the host cell culture media are encompassed in the phrase “isolated from the host cell.” A useful quantity includes an amount, including an aliquot or sample, used to screen for or monitor production, including monitoring modulation of target gene expression.

The present invention provides for the production of immunogenic agents, including an antigen, antigenic polypeptide, a metabolite, an intermediate, a viral antigen, bacterial antigen, fungal antigen, parasite antigen, virus particle, defective virus, live attenuated virus, killed virus, or vaccine. Immunogenic agents can include any immunogenic substance capable of being produced by a host cell and recovered in useful quantities, including but not limited to, polypeptides, glycoproteins and “biologics” such as a a vaccine that is synthesized from living organisms or their products, and used as a preventive, or therapeutic agent. Thus, immunogenic agents can be used for a wide range of applications, including as biotherapeutic agents, vaccines, research or diagnostic reagents, and the like.

In some embodiments, the immunogenic agent is a polypeptide. The polypeptide can be a recombinant polypeptide or a polypeptide endogenous to the host cell. In some embodiments, the polypeptide is a glycoprotein and the host cell is a mammalian cell. Non-limiting examples of polypeptides that can be produced according to methods provided herein include receptors, membrane proteins, cytokines, chemokines, hormones, enzymes, growth factors, growth factor receptors, antibodies, antibody derivatives and other immune effectors, interleukins, interferons, erythropoietin, integrins, soluble major histocompatibility complex antigens, binding proteins, transcription factors, translation factors, oncoproteins or proto-oncoproteins, muscle proteins, myeloproteins, neuroactive proteins, tumor growth suppressors, structural proteins, and blood proteins (e.g., thrombin, serum albumin, Factor VII, Factor VIII, Factor IX, Factor X, Protein C, von Willebrand factor, etc.) to which an immune response is desired.

As used herein, a polypeptide encompasses glycoproteins or other polypeptides which have undergone post-translational modification, such as deamidation, glycosylation, and the like. In some embodiments, the immunogenic agent is an aberrantly glycosylated protein. For example, many cancer antigens are known to be aberrantly glycoylated, particularly involving fucosyl residues. Moriwaki & Miyoshi, 2 World J. Heparol., 151-61 (2010). Thus, in one embodiment, the production of a cancer antigen is enhanced by modulating expression of a target gene encoding a fucosyltransferase, such as FUT8 (for example, by contacting a host CHO cell by use of a corresponding RNA effector molecule comprising an an antisense strand comprising at least 16 contiguous nucleotides (e.g., at least 17, at least 18, at least 19 nucleotides) of an oligonucleotide nucleotide having a sequence selected from the group consisting of SEQ ID NOs:209841-210227). In a particular embodiment, methods are provided for enhancing production of a fucosylated immunogen (e.g., a recombinant cancer antigen) by contacting a cell (e.g., CHO cell) with one or more RNA effector molecules that comprise at least 16 contiguous nucleotides of a nucleotide sequence (e.g., at least 17, at least 18, at least 19 nucleotides or more) to modulate fucosylation of the biological product. For example, the cell can be contacted with one or more RNA effector molecules of SEQ ID NOs:3152714-3152753, wherein the contacting modulates expression of the CHO cell fucosyltransferase (FUT8).

In one embodiment, production of the immunogenic agent is enhanced by contacting the host cell with at least one RNA effector molecule against target genes selected from the group consisting of FUT8, TSTA3, and GMDS, e.g., to modulate fucosylation. In one embodiment, at least two RNA effector molecules against target genes selected from the group consisting of FUT8, TSTA3, and GMDS are used. In one aspect of these embodiments, the host cell can be further contacted with a RNA effector molecule that targets a gene that encodes a sialytransferase, e.g., CHO cell ST3 β-galactoside-2,3-sialyltransferase 1 (SEQ ID NO:2088), ST3 β-galactoside-2,3-sialyltransferase 4 (SEQ ID NO:2167), ST3 β-galactoside-2,3-sialyltransferase 3 (SEQ ID NO:3411), ST3 β-galactoside-2,3-sialyltransferase 5 (SEQ ID NO:3484), ST6 (—N-acetyl-neuraminyl-2,3-β-galactosyl-1,3)—N-acetylgalactosaminide-2,6-sialyltransferase 6 (SEQ ID NO:4186) or ST3 β-galactoside-2,3-sialyltransferase 2 (SEQ ID NO:4319). Targeting sialyltransferases can also be advantageous in the context of altering host cell membrane-associated sialic acid viral receptors, as discussed further herein.

In one embodiment the RNA effector molecule is an siRNA having a sequence selected from the group consisting of CHO cell ST3 β-galactoside α-2,3-sialyltransferase 1 (SEQ ID NOs:681105-681454), ST3 β-galactoside α-2,3-sialyltransferase 4 (SEQ ID NOs:707535-707870), ST3 β-galactoside α-2,3-sialyltransferase 3 (SEQ ID NOs:1131123-1131445), ST3 β galactoside α-2,3-sialyltransferase 5 (SEQ ID NOs:1155324-1155711), ST6 (α-N-acetyl-neuraminyl-2,3-β-galactosyl-1,3)-N-acetylgalactosaminide α-2,6-sialyltransferase 6 (SEQ ID NOs:1391079-1391449), or ST3 β-galactoside α-2,3-sialyltransferase 2 (SEQ ID NOs: 1435989-1436317).

In other embodiments, the immunogenic agent is an immunogenic viral, bacterial, allergen, fungal, parasite, protozoan, or recombinant protein derived from an expression vector.

Another example approach for producing viral-based vaccines involves the use of attenuated live virus vaccines, which are capable of replication but are not pathogenic, and, therefore, provide lasting immunity and afford greater protection against disease. The conventional methods for producing attenuated viruses involve the chance isolation of host range mutants, many of which are temperature sensitive, e.g., the virus is passaged through unnatural hosts, and progeny viruses which are immunogenic, yet not pathogenic, are selected. Efficient vaccine production requires the growth of large quantities of virus produced in high yields from a host system. Different types of virus require different growth conditions in order to obtain acceptable yields. The host in which the virus is grown is therefore of great significance. As a function of the virus type, a virus can be grown in embryonated eggs, primary tissue culture cells, or in established cell lines.

Thus, in some embodiments of the present invention, the immunogenic agent is a viral product, for example, naturally occurring viral strains, variants or mutants; mutagenized viruses (e.g., generated by exposure to mutagens, repeated passages and/or passage in non-permissive hosts), reassortants (in the case of segmented viral genomes), and/or genetically engineered viruses (e.g., using the “reverse genetics” techniques) having the desired phenotype. The viruses of these embodiments can be attenuated; i.e., they are infectious and can replicate in vivo, but generate low titers resulting in subclinical levels of infection that are generally non-pathogenic.

Additionally, the immunogenic agent of the present invention can be derived from an intracellular parasite against which production of an immunogenic agent can be enhanced using the compositions, cells, and/or methods of the present invention, e.g., using a RNA effector molecule. For example, alternative embodiments of the present invention provide for production of a bacterial immunogen in a eukaryotic cell. These bacteria include Shigella flexneri, Listeria monocytogenes, Rickettsiae tsutsugamushi, Rickettsiae rickettsiae, Mycobacterium leprae, Mycobacterium tuberculosis, Legionella pneumophila, Chlamydia ssp. Additional embodiments of the present invention provide for production of a protozoan immunogen in a eukaryotic cell. These protozoa include Plasmodium falciparum, Tripanosoma cruzi, and Leishmania donovani.

In some embodiments, the enhancement of production of an immunogenic agent is achieved by improving viability of the cells in culture. As used herein, the term “improving cell viability” refers to an increase in cell density (e.g., as assessed by a Trypan Blue exclusion assay) or a decrease in apoptosis (e.g., as assessed using a TUNEL assay) of at least 10% in the presence of a RNA effector molecule(s) compared with the cell density or apoptosis levels in the absence of such a treatment. In some embodiments, the increase in cell density or decrease in apoptosis in response to treatment with a RNA effector molecule(s) is at least 20%, at least 30%, at least 40%, at least 50%, at least 60%, at least 70%, at least 80%, at least 90%, at least 95%, or even 100% compared to untreated cells. In some embodiments, the increase in cell density in response to treatment with a RNA effector molecule(s) is at least 2-fold, at least 5-fold, at least 10-fold, at least 20-fold, at least 50-fold, at least 100-fold, at least 1000-fold or higher than the cell density in the absence of the RNA effector molecule(s).

“Bioprocessing” as used herein is an exemplary process for the industrial-scale production of an immunogenic agent (e.g., a recombinant antigenic polypeptide) in cell culture (e.g., in a mammalian host cell), that typically includes the following steps: (a) inoculating mammalian host cells (e.g., that comprises either a virus, or a transgene that encodes a recombinant antigenic polypeptide) into a seed culture vessel containing cell culture medium and propagating the cells to reach a minimum threshold cross-seeding density; (b) transferring the propagated seed culture cells, or a portion thereof, to a large-scale bioreactor; (c) propagating the large-scale culture under conditions allowing for rapid growth and cell division until the cells reach a predetermined density; (d) maintaining the culture under conditions that disfavor continued cell growth and/or host cell division and facilitate expression of the antigenic protein or virus particles.

Steps (a) to (c) of the above method generally comprise a “growth” phase, whereas step (d) generally comprises a “production” phase. In some embodiments, fed batch culture or continuous cell culture conditions are tailored to enhance growth and division of the host cells in the growth phase and to disfavor cell growth and/or division and facilitate expression of the immunogenic agent during the production phase. For example, in some embodiments, an immunogenic agent is expressed at levels of about 1 mg/L, about 2.5 mg/L, about 5 mg/L, about 1 g/L, about 5 g/L, about 15 g/L, or higher. The rate of cell growth and/or division can be modulated by varying culture conditions, such as temperature, pH, dissolved oxygen (dO₂) and the like. For example, suitable conditions for the growth phase can include a pH of between about pH 6.5 and pH 7.5, a temperature between about 30° C. to 38° C., and a dO₂ between about 5% to 90% saturation. In some embodiments, the expression of a heterologous protein can be enhanced in the production phase by inducing a temperature shift to a lower culture temperature (e.g., from about 37° C. to about 30° C.), increasing the concentration of solutes in the cell culture medium, or adding a toxin (e.g., sodium butyrate) to the cell culture medium. In some embodiments, the expression of a heterologous protein can be enhanced in the production phase by inducing a temperature shift to about 28° C., e.g., to increase protein expression in the absence of call division (see, e.g., Example 11). A variety of additional protocols and conditions for enhancing growth and/or protein expression during the production phase are known in the art.

The host cells can be cultured in a stirred tank bioreactor system in a fed batch culture process in which the host cells and culture medium are supplied to the bioreactor initially and additional culture nutrients are fed, continuously or in discrete increments, throughout the cell culture process. The fed batch culture process can be semi-continuous, wherein periodically whole culture (including cells and medium) is removed and replaced by fresh medium. Alternatively, a simple batch culture process can be used in which all components for cell culturing (including the cells and culture medium) are supplied to the culturing vessel at the start of the process. A continuous perfusion process can also be used, in which the cells are immobilized in the culture, e.g., by filtration, encapsulation, anchoring to microcarriers, or the like, and the supernatant is continuously removed from the culturing vessel and replaced with fresh medium during the process.

In one embodiment, after the production phase the immunogenic agent is recovered from the cell culture medium using various methods known in the art. For example, recovering a secreted heterologous protein typically involves removal of host cells and debris from the medium, for example, by centrifugation or filtration. In some cases, particularly if the immunogenic agent is a protein is not secreted, protein recovery can also be performed by lysing the cultured host cells, e.g., by mechanical shear, osmotic shock, or enzymatic treatment, to release the contents of the cells into the homogenate. The protein can then be separated from subcellular fragments, insoluble materials, and the like by differential centrifugation, filtration, affinity chromatography, hydrophobic interaction chromatography, ion-exchange chromatography, size exclusion chromatography, electrophoretic procedures (e.g., preparative isoelectric focusing (IEF)), ammonium sulfate precipitation, and the like. Procedures for recovering and purifying particular types of proteins are known in the art.

In some embodiments, it is desirable to adapt cells to serum free media and adapt adherent cells to cell growth in suspension. In some embodiments, cells are adapted to grow in serum-free medium. In one aspect of the invention, adaptation of cells is facilitated by increasing cell placisity by using a RNA effector molecule that targets genes involved in control of plasticity. For example, a RNA effector targeting cell cycle regulators (e.g., cyclin kinase and others described herein) (see, e.g., Table 13, that identifies example CHO cyclin kinase target genes and exemplary siRNAs (antisense strand)); histone and DNA methylases (see Tables 1-2, that identify example CHO target genes and exemplary siRNAs (anti-sense stand)); p53 (see Table 13, that identifies example CHO target genes and exemplary siRNAs (antisense strand); and stress response proteins for example, heat shock proteins (e.g., HSP90, etc.) (see Table 15, that identifies example CHO target genes and exemplary siRNAs (antisense strand)), and the like can be used. In one embodiment, a RNA effector targets a transcript that encodes transformation related protein p53 (CHO4957.1) comprising SEQ ID NO:4957. In one embodiment, the RNA effector molecule targeting p53 comprises at least 16 contiguous nucleotides (e.g., at least 17, at least 18, at least 19 nucleotides) of an oligonucleotide nucleotide having a sequence selected from the group consisting of SEQ ID NOs:1649857-1650157.

TABLE 2
Histone Deacetylase

| SEQ ID | | | Avg | siRNA |
|---|---|---|---|---|
| NO: | consL | Description | Cov | SEQ ID Nos: |
| 1754 | 2157 | histone deacetylase 6 | 10.782 | 567757-568119 |
| 1979 | 2085 | histone deacetylase 5 | 7.779 | 644628-644970 |
| 2337 | 1975 | histone deacetylase 1 | 59.419 | 765392-765715 |
| 2781 | 1861 | histone deacetylase 3 | 24.855 | 916015-916347 |
| 3049 | 1780 | histone deacetylase 7 | 2.965 | 1007551-1007926 |
| 3374 | 1701 | histone deacetylase 2 | 14.591 | 1118498-1118826 |
| 4712 | 1390 | histone deacetylase 4 | 1.236 | 1566324-1566700 |
| 5878 | 1129 | histone deacetylase 8 | 1.863 | 1972862-1973238 |

TABLE 3
Histone Demethylase

| SEQ | | | Avg | |
|---|---|---|---|---|
| ID NO: | consL | Description | Cov | siRNA SEQ ID NOs: |
| 8124 | 593 | jumonji C domain-containing histone | 0.097 | 2740320-2740607 |
| | | demethylase 1 homolog D (S. cerevisiae) | | |
| 3143 | 1759 | KDM1 lysine (K)-specific demethylase 6B | 0.901 | 1039895-1040219 |
| 3732 | 1616 | KDM3B lysine (K)-specific demethylase 3B | 1.408 | 1238921-1239289 |
| 1277 | 2344 | lysine (K)-specific demethylase 1 | 23.583 | 404752-404996 |
| 46 | 4190 | lysine (K)-specific demethylase 2A | 3.834 | 24130-24506 |
| 804 | 2588 | lysine (K)-specific demethylase 2B | 2.962 | 249009-249279 |
| 2238 | 2001 | lysine (K)-specific demethylase 3A | 2.287 | 731689-732019 |
| 5937 | 1116 | lysine (K)-specific demethylase 4A | 0.332 | 1994536-1994923 |
| 4730 | 1387 | lysine (K)-specific demethylase 4C | 0.743 | 1572325-1572714 |
| 3157560 | 3436 | lysine (K)-specific demethylase 5A | 0.649 | 3201397-3201496 |
| 4012 | 1547 | lysine (K)-specific demethylase 5B | 0.291 | 1332770-1333138 |
| 207 | 3330 | lysine (K)-specific demethylase 5C | 4.939 | 74541-74774 |

The terms “system”, “computing device”, and “computer-based system” refer to the computer hardware, associated software, and data storage devices used to analyze the information of the present invention. In one embodiment, the computer-based systems of the present invention comprises one or more central processing units (e.g., CPU, PAL, PLA, PGA), input means (e.g., keyboard, cursor control device, touch screen), output means (e.g., computer display, printer) and data storage devices (e.g., RAM, ROM, volatile and non-volatile memory devices, hard disk drives, network attached storage, optical storage devices, magnetic storage devices, solid state storage devices). As such, any convenient computer-based system can be employed in the present invention. Further, the computing device can included an embedded system based on a combination computing hardware and associated software or firmware.

A “processor” includes any hardware and/or software combination which can perform the functions under program control. For example, any processor herein can be a programmable digital microprocessor such as available in the form of an embedded system, a programmable controller, mainframe, server or personal computer (desktop or portable). Where the processor is selectively programmable, suitable programs, software or firmware can be communicated from a remote location to the processor, or previously saved in a computer program product (such as a portable or fixed computer readable storage medium, whether magnetic, optical or solid state device based). For example, a magnetic medium or optical disk can store the program or operating instructions and can be read and transferred to each processor at its corresponding station.

“Computer readable medium” as used herein refers to any storage or transmission medium that participates in providing instructions and/or data to a computer for execution and/or processing. Examples of storage media include floppy disks, magnetic media (tape, disk), UBS, optical media (CD-ROM, DVD, Blu-Ray), solid state media, a hard disk drive, a RAM, a ROM or integrated circuit, a magneto-optical disk, or a computer readable card such as a PCMCIA card and the like, whether or not such devices are internal or external to the computer. A file containing information can be “stored” on computer readable medium, where “storing” means recording information such that it is accessible and retrievable at a later date by a computer.

With respect to computer readable media, “permanent memory” or “non-volatile memory” refers to memory that is permanently stored on a data storage medium. Permanent memory is not erased by termination of the electrical supply to a computer or processor. A computer hard-drive, ROM, CD-ROM, floppy disk and DVD are all examples of permanent memory. Random Access Memory (RAM) is an example of non-permanent or volatile memory.

To “record” or “store” data, programming or other information on a computer readable medium refers to a process for storing information, using any convenient method. Any convenient data storage structure can be chosen, based on the means used to access the stored information.

A “memory” or “memory unit” refers to any device which can store information for subsequent retrieval by a processor, and can include magnetic or optical devices (such as a hard disk, floppy disk, CD, or DVD), or solid state memory devices (such as volatile or non-volatile RAM). A memory or memory unit can have more than one physical memory device of the same or different types (for example, a memory can have multiple memory devices such as multiple hard drives or multiple solid state memory devices or some combination of hard drives and solid state memory devices).

This application describes a variety of genes, transcripts, proteins, etc. using known names for the nucleic acid sequence. To the extent a specific sequence identifier is not cross-referenced to such a name, the artisan can readily do so by known means. For example, there are numerous searchable sites such as GeneCards.org (a collaborative searchable, integrated, database of human genes that provides concise genomic, transcriptomic, genetic, proteomic, functional and disease related information on all known and predicted human genes; database developed at the Crown Human Genome Center, Department of Molecular Genetics, the Weizmann Institute of Science), and publications that form the basis of such sites. One can readily use the name to locate the sequence and using such sequence cross-reference the Sequence No. used herein. Similarly, by looking for complementary sequences of at least 15 nucleic acids identify the corresponding siRNAs to such genes.

Throughout the specification, in some cases we have given the gene abbreviation or alias of the target gene and corresponding siRNA SEQ ID NOs for that gene. In some cases we have given the full gene name of the target gene, the corresponding SEQ ID NO. for the target gene (e.g., transcript sequence) as well as example siRNA SEQ ID NOs directed against the target gene. In various embodiments of the invention, the RNA effector molecule is a siRNA that comprises an antisense strand comprising at least 16 contiguous nucleotides of a siRNA nucleotide sequence of any of the siRNA sequences identified herein by SEQ ID NO., see, e.g., Tables 1-16, 21-25, 27-30, 31, 33, 35, 37, 39, 41, 43, 45, 47, 49, 50, 51-61, 64, 65 and 66.

It should be understood that the siRNAs identified by SEQ ID NO. are often referred to herein within a range of SEQ ID NOs, e.g., from SEQ ID NOs: 2480018-2480362. The range includes all SEQ ID NOs: within the range, e.g., SEQ ID NO: 2480018, SEQ ID NO:2480019, SEQ ID NO: 2480020, etc., all the way to SEQ ID NO: 2480362.

### II. ENHANCING BIOPROCESSING

The invention provides methods for enhancing the production of immunogenic agents using the RNA effector molecules described herein. The methods generally comprise contacting a cell with a RNA effector molecule, a portion of which is complementary to a target gene, and maintaining the cell in culture (e.g., a large-scale bioreactor) for a time sufficient to modulate expression of the target gene, wherein the modulation enhances production of the immunogenic agent from the cell, and isolating the immunogenic agent from the cell. The RNA effector molecule(s) can be added to the cell culture medium used to maintain the cells under conditions that permit production of an immunogenic agent, e.g., to provide transient modulation of the target gene thereby enhancing expression of the immunogen.

As known to those of skill in the art liposome mediated delivery of siRNA using lipid polynucleotide carriers is commonly used in research applications. As described in PCT publication WO 2009/012173, however, the use of lipid polynucleotide carriers, e.g., common liposome transfection reagents, has been found to be detrimental when used in bioprocessing of protein. Polynucleotide carriers have been reported to be toxic to host cells due to toxicity such that they impair the ability of host cells to produce the desired immunogenic agent on an industrial level. In addition, polynucleotide carriers have been observed to cause adverse and unwanted changes in the phenotype of host cells, e.g., CHO cells, compromising the ability of the host cells to produce the immunogenic agent of interest. Accordingly, the artisan would expect that the use of such polynucleotide carriers would hinder a cells ability to produce a desired protein.

Surprisingly, as described herein, RNA effector molecules (e.g., targeting BAX, BAK and/or LDH) can be delivered transiently to host cells in culture by using polynucleotide carriers (e.g., lipid formulated mediated delivery) during the bioprocessing procedure in large scale cultures (e.g., 1 L and 40 L) without detrimental effects on the cells, e.g., cell viability and density is maintained. Thus, large scale production of immunogenic agents can be done, on an industrial scale, using lipid reagents to facilitate RNA effector uptake in cells when they are in culture (e.g., suspension culture), for example, resulting in transient modulation of genes that increase protein production. It should be understood, however, that embodiments of the invention are not limited to delivery of RNA effector molecules by lipid formulation mediated delivery.

In one embodiment, the production of an immunogenic agent is enhanced by contacting cultured cells with a RNA effector molecule provided herein during the production phase to modulate expression of a target gene encoding a protein that affects protein expression, post-translational modification, folding, secretion, and/or other processes related to production and/or recovery of the immunogenic agent. In further embodiments, the production of an immunogenic agent is enhanced by contacting cultured cells with a RNA effector molecule that inhibits cell growth and/or cell division during the production phase.

In some embodiments, the production of an immunogenic agent in a cultured host cell is enhanced by contacting the cell with a RNA effector molecule which modulates expression of a protein of a contaminating virus, thus reducing the contaminant's infectivity and/or viral load in the host cell. In additional embodiments, production of an immunogenic agent in a cultured host cell is enhanced by contacting the cell with a RNA effector molecule which modulates expression of a host cell protein involved in viral infection, e.g., a cell membrane ligand, or viral reproduction, thus reducing the infectivity and/or load of contaminating viruses in the host cell.

In some embodiments, host cell target genes useful for modulation include those described in Table 1 as follows:

TABLE 1
Focused Immune Response Targets

| SEQ | | | Avg | siRNA SEQ |
|---|---|---|---|---|
| ID NO: | consL | Description | Cov | ID NOs: |
| 166 | 3461 | xenotropic and polytropic retrovirus receptor 1 | 0.95 | 62021-62362 |
| 680 | 2676 | polymerase (RNA) III (DNA directed) polypeptide E | 5.84 | 211082-211316 |
| 2455 | 1943 | host cell factor C1 | 2.096 | 805085-805458 |
| 2525 | 1927 | myxovirus (influenza virus) resistance 2 | 8.118 | 829145-829432 |
| 2543 | 1922 | beclin 1, autophagy related | 22.681 | 835365-835694 |
| 3179 | 1750 | polymerase (RNA) III (DNA directed) | 5.685 | 1052412-1052729 |
| | | polypeptide D | | |
| 3259 | 1732 | polymerase (RNA) III (DNA directed) polypeptide C | 15.023 | 1079448-1079786 |
| 3885 | 1577 | SWI/SNF related, matrix associated, actin dependent | 11.687 | 1290692-1291012 |
| | | regulator of chromatin, subfamily b, member 1 | | |
| 4201 | 1500 | eukaryotic translation initiation factor 2 α kinase 3 | 2.46 | 1396283-1396617 |
| 4256 | 1491 | polymerase (RNA) III (DNA directed) | 1.005 | 1414629-1414949 |
| | | polypeptide B | | |
| 4266 | 1488 | tumor susceptibility gene 101 | 23.4 | 1417992-1418306 |
| 4832 | 1362 | mitochondrial antiviral signaling protein | 1.615 | 1607184-1607527 |
| 5436 | 1229 | polymerase (RNA) III (DNA directed) | 0.45 | 1814931-1815240 |
| | | polypeptide F | | |
| 5608 | 1188 | caspase 12 | 0.856 | 1875252-1875646 |
| 5618 | 1187 | myeloid differentiation primary response gene 88 | 1.629 | 1878827-1879137 |
| 5799 | 1146 | lysosomal trafficking regulator | 0.206 | 1944185-1944541 |
| 5948 | 1114 | interferon regulatory factor 7 | 2.718 | 1998635-1999022 |
| 7260 | 823 | DEAD (Asp-Glu-Ala-Asp) box polypeptide 58 | 0.166 | 2454994-2455378 |
| 7439 | 778 | B-cell leukemia/lymphoma 2 | 0.149 | 2513854-2514170 |
| 7465 | 772 | zinc finger CCCH type, antiviral 1 | 0.346 | 2522447-2522771 |
| 7670 | 721 | myxovirus (influenza virus) resistance 1 | 0.687 | 2588615-2588951 |
| 7683 | 718 | toll-like receptor 3 | 0.226 | 2593179-2593525 |
| 7716 | 710 | polymerase (RNA) III (DNA directed) | 2.352 | 2604412-2604804 |
| | | polypeptide H | | |
| 7764 | 698 | polymerase (RNA) III (DNA directed) | 0.231 | 2620918-2621272 |
| | | polypeptide G | | |
| 7929 | 651 | interleukin 23, α subunit p19 | 0.852 | 2676772-2677097 |
| 8096 | 601 | barrier to autointegration factor 1 | 10.185 | 2731441-2731749 |
| 8245 | 562 | calcitonin gene-related peptide-receptor | 0.987 | 2778256-2778534 |
| | | component protein | | |
| 8318 | 541 | T-cell specific GTPase | 0.193 | 2802893-2803167 |
| 8531 | 490 | interleukin 15 | 1.901 | 2874576-2874952 |
| 9014 | 389 | polymerase (RNA) III (DNA directed) | 0.509 | 3021834-3022134 |
| | | polypeptide K | | |
| 9395 | 285 | 2′-5′ oligoadenylate synthetase 1B | 0.156 | 3108340-3108557 |
| 9402 | 282 | ISG15 ubiquitin-like modifier | 1.263 | 3109784-3109974 |
| 9724 | 148 | ATP-binding cassette, sub-family C | 0.096 | 3149990-3150001 |
| | | (CFTR/MRP), member 9 | | |
| 9741 | 139 | NLR family, pyrin domain containing 3 | 0.035 | 3150878-3150975 |
| 3157613 | 530 | radical S-adenosyl Met domain containing 2 | 0.148 | 3252217-3252316 |

In some embodiments, the enhancement of production of an immunogenic agent upon modulation of a target gene is detected by monitoring one or more measurable bioprocess parameters, such as a parameter selected from the group consisting of: cell density, pH, oxygen levels, glucose levels, lactic acid levels, temperature, and protein production. Protein production can be measured as specific productivity (SP) (the concentration of a product, such as a heterologously expressed polypeptide, in solution) and can be expressed as mg/L or g/L; in the alternative, specific productivity can be expressed as pg/cell/day. An increase in SP can refer to an absolute or relative increase in the concentration of a product produced under two defined set of conditions (e.g., when compared with controls not treated with RNA effector molecule(s)).

In some embodiments, the enhancement of production of an immunogenic agent, upon modulation of a target gene, is detected by monitoring one or more measurable bioprocess parameters, such as cell density, medium pH, oxygen levels, glucose levels, lactic acid levels, temperature, viral protein, or viral particle production. For example, protein production can be measured as specific productivity (SP) (the concentration of a product in solution) and can be expressed as mg/L or g/L; in the alternative, specific productivity can be expressed as pg/cell/day. An increase in SP can refer to an absolute or relative increase in the concentration of an immunogenic agent produced under two defined set of conditions. Alternatively, viral particle products can be titrated by well known plaque assays, measured as plaque forming units per mL (PFU/mL).

In some embodiments, RNA effector compositions include two or more RNA effector molecules, e.g., comprise two, three, four or more RNA effector molecules. In various embodiments, the two or more RNA effector molecules are capable of modulating expression of the same target gene and/or one or more additional target genes. Advantageously, certain compositions comprising multiple RNA effector molecules are more effective in enhancing production of an immunogenic agent, or one or more aspects of such production, than separate compositions comprising the individual RNA effector molecules.

In other embodiments, a plurality of different RNA effector molecules are contacted with the cell culture and permit modulation of one or more target genes. In one embodiment, at least one of the plurality of different RNA effector molecules is a RNA effector molecule that modulates expression of glutaminase, glutamine dehydrogenase, or LDH. In another embodiment, RNA effector molecules targeting Bax and Bak are co-administered to a cell culture during production of the immunogenic agent and can optionally contain at least one additional RNA effector molecule or agent. In another embodiment, a plurality of different RNA effector molecules is contacted with the cells in culture to permit modulation of Bax, Bak and LDH expression. In another embodiment, a plurality of different RNA effector molecules is contacted with the cells in culture to permit modulation of expression of Bax and Bak, as well as glutaminase and/or glutamine dehydrogenase.

When a plurality of different RNA effector molecules are used to modulate expression of one or more target genes the plurality of RNA effector molecules can be contacted with cells simultaneously or separately. In addition, each RNA effector molecule can have its own dosage regimen. For example, one can prepare a composition comprising a plurality of RNA effector molecules are contacted with a cell. Alternatively, one can administer one RNA effector molecule at a time to the cell culture. In this manner, one can easily tailor the average percent inhibition desired for each target gene by altering the frequency of administration of a particular RNA effector molecule. For example, strong inhibition (e.g., >80% inhibition) of lactate dehydrogenase (LDH) may not always be necessary to significantly improve production of an immunogenic agent and under some conditions it may be preferable to have some residual LDH activity. Thus, one may desire to contact a cell with a RNA effector molecule targeting LDH at a lower frequency (e.g., less often) or at a lower dosage (e.g., lower multiples over the IC₅₀) than the dosage for other RNA effector molecules. Contacting a cell with each RNA effector molecule separately can also prevent interactions between RNA effector molecules that can reduce efficiency of target gene modulation. For ease of use and to prevent potential contamination it may be preferred to administer a cocktail of different RNA effector molecules, thereby reducing the number of doses required and minimizing the chance of introducing a contaminant to the cell or cell culture.

In some embodiments, the production of an immunogenic agent is enhanced by contacting cultured cells with a RNA effector molecule provided herein during the growth phase to modulate expression of a target gene encoding a protein that affects cell growth, cell division, cell viability, apoptosis, nutrient handling, and/or other properties related to cell growth and/or division. In further embodiments, the production of a heterologous protein is enhanced by contacting cultured cells with a RNA effector molecule which transiently inhibits expression of the heterologous protein during the growth phase.

In yet further embodiments, the modulation of expression (e.g., inhibition) of a target gene by a RNA effector molecule can be alleviated by contacting the cell with second RNA effector molecule, wherein at least a portion of the second RNA effector molecule is complementary to a target gene encoding a protein that mediates RNAi in the host cell. For example, the modulation of expression of a target gene can be alleviated by contacting the cell with a RNA effector molecule that inhibits expression of an argonaute protein (e.g., Argonaute-2) or other component of the RNAi pathway of the cell. In one embodiment, the immunogenic agent is a recombinant protein and expression of the product is transiently inhibited by contacting the cell with a first RNA effector molecule targeted to the transgene encoding the immunogenic agent. The inhibition of expression of the immunogenic agent is then alleviated by contacting the host cell with a second RNA effector molecule targeted against a gene encoding a protein of the RNAi pathway of the cell.

Host Cell Immune Response

In additional embodiments, production of an immunogenic agent in a host cell is further enhanced by introducing a RNA effector molecule that modulates expression of a host cell protein involved in microbial infection or replication such that the infectivity, load, and/or production of the immunogenic agent is increased. Modulating a host cell immune response can also be beneficial in the production of certain immunogenic agents that are themselves involved in modulating the immune response (e.g., influenza and the like).

For example, several human, mammalian and avian viruses are introduced into and/or cultivated in cells for either virus production or heterologous protein expression (e.g., ultimately for vaccine production). Infection or transfection results in the accumulation of an immunogenic agent, such as live virus particles, which can be collected from either cells or cell media after a suitable incubation period. For example, the standard method of vaccine production consists of culturing cells, infecting with a live virus (e.g., rotavirus, influenza, yellow fever), incubation, harvesting of cells or cell media, downstream processing, and filling and finishing. For the classic inactived influenza vaccine, purification, inactivation, and stabilization of this harvested immunogenic agent yields vaccine product, which techniques are well known in the art.

Recombinant DNA technology and genetic engineering techniques, in theory, can afford a superior approach to producing an attenuated virus because specific mutations are deliberately engineered into the viral genome. The genetic alterations required for attenuation of viruses are not always predictable, however. In general, the attempts to use recombinant DNA technology to engineer viral vaccines have been directed to the production of subunit vaccines which contain only the protein subunits of the pathogen involved in the immune response, expressed in recombinant viral vectors such as vaccinia virus or baculovirus. More recently, recombinant DNA techniques have been utilized to produce herpes virus deletion mutants or polioviruses that mimic attenuated viruses found in nature or known host range mutants.

The yield of an immunogenic agent, such as an attenuated live influenza virus or an immunomodulatory polypeptide, made in a host cell can be adversely affected by the immune response of the host cell, e.g., the interferon response of the host cell in which the virus or viral vector is replicated. Additionally, the infected host cell(s) can become apoptotic before viral yield is maximized. Thus, although these attenuated viruses are immunogenic and non-pathogenic, they are often difficult to propagate in conventional cell substrates for the purposes of making vaccines. Hence, some embodiments of the present invention provide for compositions and methods using a RNA effector molecules to modulate the expression of adverse host cell responses and therefore increase yield. For example, some embodiments of the present invention relate to contacting a cell with a RNAi-based product siRNA prior to, during or after the viral or vector administration, to inhibit cellular and anti-viral processes that compromise the yield and quality of the product harvest.

The use of cell-based bioprocesses for the manufacture of immunogenic agents is enhanced, in some embodiments, by modulating expression of a target gene affecting the host cell's reaction to viral infection. This approach is useful where the immunogenic agent is viral or otherwise immunomodulatory, or where viral vectors are used to introduce heterologous proteins into the host cell.

For example, in some embodiments the target gene is a cell interferon protein or a protein associated with interferon signaling. In particular, the gene can be an interferon gene such as IFN-α(e.g., Gallus IFN-α, GeneID: 396398); IFN-β (e.g., Gallus IFN-β, GeneID: 554219); or IFN-γ (e.g., Gallus IFN-γ, GeneID: 396054). Thus, for example, IFN-β expression can be modulated by use of corresponding RNA effector molecule having an antisense strand comprising at least 16 contiguous nucleotides (e.g., at least 17, at least 18, at least 19 nucleotides) of an oligonucleotide nucleotide having a sequence selected from the group consisting of SEQ ID NOs:3156155-3156180 (Gallus, sense), SEQ ID NOs:3156181-3156206 (Gallus, antisense), SEQ ID NOs:3155493-3155540 (Canis, sense), SEQ ID NOs:3155445-3155492 (Canis, antisense), depending on the cultured cell.

Alternatively, the target gene can be an interferon receptor such as IFNAR1 (interferon α, β and ω receptor 1) (e.g., Gallus IFNAR1, GeneID: 395665), IFNAR2 (interferon α, β and ω receptor 2) (e.g., Gallus IFNAR2, GeneID: 395664), IFNGR1 (interferon γ receptor 1) (e.g., Gallus IFNGR1, GeneID: 421685) or IFNGR2 (interferon γ receptor 2 (interferon γ transducer 1)) (e.g., Gallus IFNGR2, GeneID: 418502). Thus, for example, IFNAR1 expression can be modulated by use of corresponding RNA effector molecule having an antisense strand comprising at least 16 contiguous nucleotides (e.g., at least 17, at least 18, at least 19 nucleotides) of an oligonucleotide nucleotide having a sequence selected from the group consisting of SEQ ID NOs:2436536-2436863 (CHO cell, antisense), SEQ ID NOs:3154605-3154633 (Gallus, sense), SEQ ID NOs:3154634-3154662 (Gallus, antisense), SEQ ID NOs:3155397-3155444 (Canis, sense), SEQ ID NOs:3155445-3155492 (Canis, antisense), depending on the cultured cell.

In some embodiments, the gene can be associated with interferon signaling such as STAT-1 (signal transducer and activator of transcription 1) (e.g., Gallus Stat1, GeneID: 424044), STAT-2, STAT-3 (e.g., Gallus Stat3, GeneID:420027), STAT-4 (e.g., Gallus Stat4, GeneID: 768406), STAT-5 (e.g., Gallus Stat5, GeneID: 395556; JAK-1 (Janus kinase 1) (e.g., Gallus Jak1, GeneID: 395681; JAK-2 (e.g., Gallus Jak2, GeneID: 374199), JAK-3 (e.g., Gallus Jak3, GeneID: 395845), IRF1 (interferon regulatory factor 1) (e.g., Gallus IRF1, GeneID: 396384), IRF2 (e.g., Gallus GeneID: 396115), IRF3, IRF4 (e.g., Gallus GeneID: 374179), IRF5 (e.g., Gallus GeneID: 430409), IRF6 (e.g., Gallus GeneID: 419863), IRF7 (e.g., Gallus GeneID: 396330), IRF8 (e.g., Gallus GeneID:396385), IRF 9, or IRF10 (e.g., Gallus GeneID: 395243).

Thus, for example, IRF3 expression can be modulated by use of corresponding RNA effector molecule having an antisense strand comprising at least 16 contiguous nucleotides (e.g., at least 17, at least 18, at least 19 nucleotides) of an oligonucleotide nucleotide having a sequence selected from the group consisting of SEQ ID NOs:1430473-1430786 (CHO cell, antisense), SEQ ID NOs:3288948-3289249 (Gallus, sense), SEQ ID NOs:3289250-3289551 (Gallus, antisense), SEQ ID NOs:3290142-3290445 (Canis, sense), SEQ ID NOs:320446-320749 (Canis, antisense), depending on the cultured cell.

Similarly, the target gene can encode an interferon-induced protein such as 2′,5′ oligoadenylate synthetases (2-50AS); an interferon-induced antiviral protein; RNaseL (ribonuclease L (2′,5′-oligoisoadenylate synthetase-dependent) (e.g., Gallus GeneID: 424410 (Silverman et al., 14 J. Interferon Res. 101-04 (1994)); dsRNA-dependent protein kinase (PKR) aka: eukaryotic translation initiation factor 2-α kinase 2 (EIF2AK2) (Li et al., 106 PNAS 16410-05 (2009)); Mx (MX1 myxovirus (influenza virus) resistance 1, interferon-inducible protein p78) (e.g., Gallus MX, GeneID: 395313; Haller et al., 9 Microbes Infect. 1636-43 (2007)); IFITM1, IFITM2, IFITM3 (Brass et al., 139 Cell 1243-54 (2009)); Proinflammatory cytokines; MYD88 (myeloid differentiation primary response gene) up-regulated upon viral challenge (e.g., Gallus Myd88, GeneID: 420420); or TRIF (toll-like receptor adaptor molecule 1) (e.g., Gallus TRIF, GeneID: 100008585), Hghighi et al., Clin. Vacc. Immunol. (Jan. 13, 2010).

Thus, for example, MX1 expression can be modulated by use of corresponding RNA effector molecule having an antisense strand comprising at least 16 contiguous nucleotides (e.g., at least 17, at least 18, at least 19 nucleotides) of an oligonucleotide nucleotide having a sequence selected from the group consisting of SEQ ID NOs:2588615-2588951 (CHO cell, antisense), SEQ ID NOs:326682-3286975 (Gallus, sense), SEQ ID NOs:3286976-3287269 (Gallus, antisense), SEQ ID NOs:3286132-3286406 (Canis, sense), SEQ ID NOs:3286407-3286681 (Canis, antisense), depending on the cultured cell.

Also, for example IFTM1 expression can be modulated by use of corresponding RNA effector molecule having an oligonucleotide strand comprising at least 16 contiguous nucleotides (e.g., at least 17, at least 18, at least 19 nucleotides) of an oligonucleotide nucleotide having a sequence selected from the group consisting of SEQ ID NOs:3155115-3155161 (Canis, sense), SEQ ID NOs:3155162-3155208 (Canis, antisense).

Additionally, IFITM2 expression can be modulated by use of corresponding RNA effector molecule having an oligonucleotide strand comprising at least 16 contiguous nucleotides (e.g., at least 17, at least 18, at least 19 nucleotides) of an oligonucleotide nucleotide having a sequence selected from the group consisting of SEQ ID NOs:3156587-3156633 (CHO cell, sense), SEQ ID NOs:3156634-3156680 (CHO cell, antisense), SEQ ID NOs:2685171-2685550 (CHO cell, antisense), SEQ ID NOs:3155209-3155255 (Canis, sense), SEQ ID NOs:3155256-3155302 (Canis, antisense), depending on the cultured cell.

Likewise, IFITM3 expression can be modulated by use of corresponding RNA effector molecule having an antisense strand comprising at least 16 contiguous nucleotides (e.g., at least 17, at least 18, at least 19 nucleotides) of an oligonucleotide having a sequence selected from the group consisting of SEQ ID NOs:3156681-3156727 (CHO cell, sense), SEQ ID NOs:3156728-3156774 (CHO cell, antisense), SEQ ID NOs:2696169-2696546 (CHO cell, antisense), SEQ ID NOs:3155303-3155349 (Canis, sense), SEQ ID NOs:3155350-3155350 (Canis, antisense), depending on the cultured cell.

Further regarding example interferon-induced expression, PKR (EIF2AK2) expression can be modulated by use of corresponding RNA effector molecule having an antisense strand comprising at least 16 contiguous nucleotides (e.g., at least 17, at least 18, at least 19 nucleotides) of an oligonucleotide nucleotide having a sequence selected from Tables 67 and 68, as follows:

TABLE 67
Example target PKR (EIF2AK2) oligonucletides

| Gallus PKR Sense | Gallus PKR Antisense |
|---|---|
| CCACUGAGUGAUUCAGCCU | AGGCUGAAUCACUCAGUGG |
| GGUACAGGCGUUGGUAAGA | UCUUACCAACGCCUGUACC |
| CAGGCGUUGGUAAGAGUAA | UUACUCUUACCAACGCCUG |
| GAAUGUGCAUACUUCGGAU | AUCCGAAGUAUGCACAUUC |
| CAUACUUCGGAUGUAGUGA | UCACUACAUCCGAAGUAUG |
| GACAUUGCAGCUAGUUGAU | AUCAACUAGCUGCAAUGUC |
| CAUUGCAGCUAGUUGAUUA | UAAUCAACUAGCUGCAAUG |
| CCACGCUCCAAUGUAUUCU | AGAAUACAUUGGAGCGUGG |
| GUAAUUAGUGGUCAUGUAU | AUACAUGACCACUAAUUAC |
| CAUGAACUCAGUAAUUCCU | AGGAAUUACUGAGUUCAUG |
| GAGUCAUGGGGUAUUACCU | AGGUAAUACCCCAUGACUC |
| GGUAUUACCUUUAAAGACU | AGUCUUUAAAGGUAAUACC |
| GAAAGACAUGUCCCUAUCU | AGAUAGGGACAUGUCUUUC |
| GAGCCUUCAAAUUGUCGGA | UCCGACAAUUUGAAGGCUC |
| GAGUAUUGGCACCUAAUUU | AAAUUAGGUGCCAAUACUC |
| GGUUUCGUCAGCAGUAUAA | UUAUACUGCUGACGAAACC |
| CUAUGCAAUCAAACGAGUU | AACUCGUUUGAUUGCAUAG |
| GUUAAUAAAUAGGAACGUA | UACGUUCCUAUUUAUUAAC |
| GCUCGCGAAUCUUGAACAU | AUGUUCAAGAUUCGCGAGC |
| CGCGAAUCUUGAACAUGAA | UUCAUGUUCAAGAUUCGCG |
| GAAUUCUAUCGUAGCUGUU | AACAGCUACGAUAGAAUUC |
| GAAUAUAUUCCUAUCAUAU | AUAUGAUAGGAAUAUAUUC |
| CUUUGGUCUCGUGACUUCU | AGAAGUCACGAGACCAAAG |
| CCCUCUGACUAAGAACCGA | UCGGUUCUUAGUCAGAGGG |
| GAGGAACACAGUCAUAUAU | AUAUAUGACUGUGUUCCUC |
| GAUAUGGAAAGGAAGUAGA | UCUACUUCCUUUCCAUAUC |
| GGUAUGGCAGGAUGUUAGA | UCUAACAUCCUGCCAUACC |
| CCAGGUACCCAUAAUCAAA | UUUGAUUAUGGGUACCUGG |
| GACAACUCGCAUAAAGCUU | AAGCUUUAUGCGAGUUGUC |
| CACUUCUUUUAGGUGAACU | AGUUCACCUAAAAGAAGUG |
| CCUUAAGUAUUUAGCUUUU | AAAAGCUAAAUACUUAAGG |
| GUUCUUCCUUAUAGGAACA | UGUUCCUAUAAGGAAGAAC |
| CAGGUAGGGUCCUCUUAAU | AUUAAGAGGACCCUACCUG |
| GUAGGGUCCUCUUAAUACA | UGUAUUAAGAGGACCCUAC |
| CUCCUAUACAGUACGGUUU | AAACCGUACUGUAUAGGAG |
| CUAUACAGUACGGUUUUAA | UUAAAACCGUACUGUAUAG |
| GUACGGUUUUAAUCGCCUA | UAGGCGAUUAAAACCGUAC |
| GGUUUUAAUCGCCUAUUAU | AUAAUAGGCGAUUAAAACC |
| GAUUAUAGGUGUACCUGAA | UUCAGGUACACCUAUAAUC |
| GUCAGCUCAACAUAAGGUA | UACCUUAUGUUGAGCUGAC |
| CUGAUUGACCGUUACUCUU | AAGAGUAACGGUCAAUCAG |
| GACCGUUACUCUUUGGUUA | UAACCAAAGAGUAACGGUC |
| CGUUACUCUUUGGUUAUAU | AUAUAACCAAAGAGUAACG |
| GGUUAUAUACUUAAGAGAU | AUCUCUUAAGUAUAUAACC |
| CUUAAGAGAUUUCUCGUUU | AAACGAGAAAUCUCUUAAG |
| GAUUUCUCGUUUGACUAAA | UUUAGUCAAACGAGAAAUC |
| CUCGUUUGACUAAAUAAGA | UCUUAUUUAGUCAAACGAG |

TABLE 68
Example target PKR (EIF2AK2) oligonucletides

| Canis PKR Sense | Canis PKR Antisense |
|---|---|
| CAGAAAGGUACUUAAGUAU | AUACUUAAGUACCUUUCUG |
| AGAAAGGUACUUAAGUAUA | UAUACUUAAGUACCUUUCU |
| AAAGGUACUUAAGUAUAAU | AUUAUACUUAAGUACCUUU |
| UACUUAAGUAUAAUGAACU | AGUUCAUUAUACUUAAGUA |
| AAGUAUAAUGAACUGUCUA | UAGACAGUUCAUUAUACUU |
| GGACCUGCACAUAACUUAA | UUAAGUUAUGUGCAGGUCC |
| ACUUAAGAUUUACAUUCCA | UGGAAUGUAAAUCUUAAGU |
| AGCCAAAUUAGCUCUUGAA | UUCAAGAGCUAAUUUGGCU |
| AAACAAGGCGGUUAGUUCU | AGAACUAACCGCCUUGUUU |
| UUAGAAGGCGUUGGGAAUU | AAUUCCCAACGCCUUCUAA |
| UAGAAGGCGUUGGGAAUUA | UAAUUCCCAACGCCUUCUA |
| AUUACAUAGGCCGUAUGAA | UUCAUACGGCCUAUGUAAU |
| UUACAUAGGCCGUAUGAAU | AUUCAUACGGCCUAUGUAA |
| UACAUAGGCCGUAUGAAUA | UAUUCAUACGGCCUAUGUA |
| GAAGGAACAACUAUCUGUA | UACAGAUAGUUGUUCCUUC |
| AGAAAGAUUUCAUUGCAGA | UCUGCAAUGAAAUCUUUCU |
| ACAUUUGGCUGCUAAAUUU | AAAUUUAGCAGCCAAAUGU |
| UUGCAUAUGAACAGAUACA | UGUAUCUGUUCAUAUGCAA |
| AUUGUAACAGGGACAAUGU | ACAUUGUCCCUGUUACAAU |
| CUCUGAGCAAUGCCAGAUA | UAUCUGGCAUUGCUCAGAG |
| ACACAGUGGAACUCAGGUU | AACCUGAGUUCCACUGUGU |
| GAAAUAGAACCAAUUGGCU | AGCCAAUUGGUUCUAUUUC |
| AAUAGAACCAAUUGGCUCA | UGAGCCAAUUGGUUCUAUU |
| GCUCAGGUGGAUAUGGUCA | UGACCAUAUCCACCUGAGC |
| GAUUUAUGUUAUUAAACGU | ACGUUUAAUAACAUAAAUC |
| UUUAUGUUAUUAAACGUGU | ACACGUUUAAUAACAUAAA |
| UAUGUUAUUAAACGUGUUA | UAACACGUUUAAUAACAUA |
| AUGUUAUUAAACGUGUUAA | UUAACACGUUUAAUAACAU |
| UGUUAUUAAACGUGUUAAA | UUUAACACGUUUAAUAACA |
| AAGGUAGAACGGGAAGUAA | UUACUUCCCGUUCUACCUU |
| AGCGCUUGAUCACGUAAAU | AUUUACGUGAUCAAGCGCU |
| GCGCUUGAUCACGUAAAUA | UAUUUACGUGAUCAAGCGC |
| CGCUUGAUCACGUAAAUAU | AUAUUUACGUGAUCAAGCG |
| AUCACGUAAAUAUCGUGCA | UGCACGAUAUUUACGUGAU |
| UAUCGUGCACUACCGUAGU | ACUACGGUAGUGCACGAUA |
| CCUUCAAGAACAACUAAGU | ACUUAGUUGUUCUUGAAGG |
| UCUGUGAUAAAGGAACAUU | AAUGUUCCUUUAUCACAGA |
| CAUUGGAGCAAUGGAUUGA | UCAAUCCAUUGCUCCAAUG |
| GGCUAAUUCUUGCAGAACU | AGUUCUGCAAGAAUUAGCC |
| UACAUAUGUCCCACUGUUU | AAACAGUGGGACAUAUGUA |
| CUAAGGGCUGGCAAGUUCU | AGAACUUGCCAGCCCUUAG |
| ACUUGAGCCCAUGAAACGA | UCGUUUCAUGGGCUCAAGU |
| GCCCAUGAAACGACCUAAU | AUUAGGUCGUUUCAUGGGC |
| CAUGAAACGACCUAAUGCA | UGCAUUAGGUCGUUUCAUG |
| GAAACGACCUAAUGCAUCU | AGAUGCAUUAGGUCGUUUC |
| AUAUUAGAGCCCUUCUAAA | UUUAGAAGGGCUCUAAUAU |
| UCUUCUAGGGUAUUUACCU | AGGUAAAUACCCUAGAAGA |

In another embodiment, the immunogenic agent is produced by a cell transfected with one or more retroviral vectors. Upon transfection with a first retroviral vector, expression of the retroviral vector Env and/or Gag molecule is transiently inhibited by contacting the cell with a first RNA effector molecule (i.e., targeting the env gene or gag gene), allowing more efficient transfection with a second retroviral vector. For example, a first retroviral vector can encode a first peptide and a second retroviral vector can encode a second peptide (such that the recombinant immunogenic agent contains both peptides). Additionally, the inhibition of expression can be alleviated by introducing into the cell an additionally RNA effector molecule targeted against a gene encoding a protein of the RNAi pathway.

In some embodiments, the target gene is a regulatory element or gene of an endogenous retrovirus (ERV) of the cell. For example, in particular embodiments the target gene can encode an ERV LTR, env protein, or gag protein. In some embodiments, the target gene is a gene of a latent virus such as a herpesvirus, adenovirus, vesivirus, or circovirus. In particular embodiments, for example, the target gene can encode a polypeptide or protein, such as a latent HSV glycoprotein D or PCV-1 Rep protein (described elsewhere herein). Provided herein in Table 64 are exemplary RNA effector molecules for targeting PCV-1:

TABLE 64
Duplexes targeting PCV-1 with modified nucleotides

| Duplex | | |
|---|---|---|
| No | Sense | Antisense |
| 1 | uAGAAAuAAGuGGuGGGAudTsdT | AAcACCcACCUCUuAUGGGdTsdT |
| 2 | AAuAAGuGGuGGGAuGGAudTsdT | uAAGGGUGAAcACCcACCUdTsdT |
| 3 | AuAAGuGGuGGGAuGGAuAdTsdT | UuAAGGGUGAAcACCcACCdTsdT |
| 4 | uAAGuGGuGGGAuGGAuAudTsdT | AUuAAGGGUGAAcACCcACdTsdT |
| 5 | GuGGuGGGAuGGAuAucAudTsdT | uAUuAAGGGUGAAcACCcAdTsdT |
| 6 | GGAuGGAuAucAuGGAGAAdTsdT | UuAUuAAGGGUGAAcACCCdTsdT |
| 7 | uGGAuAucAuGGAGAAGAAdTsdT | AAGCUCCCGuAUUUUGUUUdTsdT |
| 8 | AuAucAuGGAGAAGAAGuudTsdT | AAGGGAGAUUGGAAGCUCCdTsdT |
| 9 | ucAuGGAGAAGAAGuuGuudTsdT | UUCCUCUCCGcAAAcAAAAdTsdT |
| 10 | uGGAGAAGAAGuuGuuGuudTsdT | AAACCUUCCUCUCCGcAAAdTsdT |
| 11 | GGAGAAGAAGuuGuuGuuudTsdT | UUCcAAACCUUCCUCUCCGdTsdT |
| 12 | GAGAAGAAGuuGuuGuuuudTsdT | uACCCUCUUCcAAACCUUCdTsdT |
| 13 | AGAAGuuGuuGuuuuGGAudTsdT | UUCuACCCUCUUCcAAACCdTsdT |
| 14 | AGuuGuuGuuuuGGAuGAudTsdT | AAUUCGcAAACCCCUGGAGdTsdT |
| 15 | GuuGuuGuuuuGGAuGAuudTsdT | AAAUUCGcAAACCCCUGGAdTsdT |
| 16 | uuuuAuGGcuGGuuAccuudTsdT | uAGcAAAAUUCGcAAACCCdTsdT |
| 17 | uGGcuGGuuAccuuGGGAudTsdT | UUCUuAGcAAAAUUCGcAAdTsdT |
| 18 | cuGGuuAccuuGGGAuGAudTsdT | AAGUCUGCUUCUuAGcAAAdTsdT |
| 19 | GAGAcuGuGuGAccGGuAudTsdT | AAAGUCUGCUUCUuAGcAAdTsdT |
| 20 | cuGuGuGAccGGuAuccAudTsdT | AAAAGUCUGCUUCUuAGcAdTsdT |
| 21 | uGuGuGAccGGuAuccAuudTsdT | uAAAAGUCUGCUUCUuAGCdTsdT |
| 22 | ccGGuAuccAuuGAcuGuAdTsdT | UuAAAAGUCUGCUUCUuAGdTsdT |
| 23 | ccAuuGAcuGuAGAGAcuAdTsdT | UUcACCUUGUuAAAAGUCUdTsdT |
| 24 | GuAuuuuGAuuAccAGcAAdTsdT | uACcACUUcACCUUGUuAAdTsdT |
| 25 | uAuuuuGAuuAccAGcAAudTsdT | AuACcACUUcACCUUGUuAdTsdT |
| 26 | cAGGAAuGGuAcuccucAAdTsdT | AAuACcACUUcACCUUGUUdTsdT |
| 27 | cAGcuGuAGAAGcucucuAdTsdT | AAAuACcACUUcACCUUGUdTsdT |
| 28 | AGcuGuAGAAGcucucuAudTsdT | UUCGCUUUCUCGAUGUGGCdTsdT |
| 29 | uAucGGAGGAuuAcuAcuudTsdT | UUCCUUUCGCUUUCUCGAUdTsdT |
| 30 | AucGGAGGAuuAcuAcuuudTsdT | UuAUUCUGCUGGUCGGUUCdTsdT |
| 31 | GAGGAuuAcuAcuuuGcAAdTsdT | UUCUUuAUUCUGCUGGUCGdTsdT |
| 32 | AGGAuuAcuAcuuuGcAAudTsdT | uACUGcAGuAUUCUUuAUUdTsdT |
| 33 | cuAcuuuGcAAuuuuGGAAdTsdT | UuACUGcAGuAUUCUUuAUdTsdT |
| 34 | uuGGAAGAcuGcuGGAGAAdTsdT | UUuACUGcAGuAUUCUUuAdTsdT |
| 35 | AAGAcuGcuGGAGAAcAAudTsdT | AUGUGGCCUUCUUuACUGCdTsdT |
| 36 | AGAAcAAuccAcGGAGGuAdTsdT | uAUGUGGCCUUCUUuACUGdTsdT |
| 37 | AcccGAAGGccGAuuuGAAdTsdT | AAGuAUGUGGCCUUCUUuAdTsdT |
| 38 | uGcccuuuucccAuAuAAAdTsdT | uAAGuAUGUGGCCUUCUUUdTsdT |

In some embodiments, the target gene is an endogenous non-ERV gene. For example, the target gene can encode the immunogenic agent, or a portion thereof, when the immunogenic agent is a polypeptide.

Production of an immunogenic agent can also be enhanced by reducing the expression of a protein that binds to the immunogenic agent or its vector. For example, in producing a recombinant protein it can be advantageous to reduce or inhibit expression of a receptor/ligand produced by an ERV, so that its expression in the host cell does not inhibit super-infection by the recombinant vector. It is known to a skilled artisan that a receptor can be a cell surface receptor or an internal (e.g., nuclear) receptor. The expression of the binding partner can be modulated by contacting the host cell with a RNA effector molecule directed at the receptor gene according to methods described herein.

In additional embodiments, the target gene is a cell protein that mediates viral infectivity, such as TLR3 that detects dsRNA (e.g., Gallus TLR3, GeneID: 422720), TLR7 that detects ssRNA (e.g., Gallus TLR7, GeneID: 418638), TLR21, that recognizes unmethylated DNA with CpG motifs (e.g., Gallus T1r3, GeneID: 415623), RIG-1 involved with viral sensing (Myong et al., 323 Science 1070-74 (2009)); LPGP2 and other RIG-1-like receptors, which are positive regulators of viral sensing (Satoh et al., 107 PNAS 1261-62 (2010); Nakhaei et al., 2009); TRIM25 (e.g., Gallus Trim25, GeneID: 417401; Gack et al., 5 Cell Host Microb. 439-49 (2009)); or MAVS/VISA/IPS-1/Gardif (MAVS), which interacts with RIG-1 to initiate an antiviral signaling cascade (see Cui et al., 29 Mol. Cell. 169-79 (2008); Kawai et al., 6 Nat. Immunol. 981-88 (2005)).

Thus, for example, TLR3 expression can be modulated by use of corresponding RNA effector molecule(s) having an antisense strand comprising at least 16 contiguous nucleotides (e.g., at least 17, at least 18, at least 19 nucleotides) of an oligonucleotide nucleotide having a sequence selected from the group consisting of SEQ ID NOs:3156491-3156538 (CHO cell, sense), SEQ ID NOs:3156539-3156586 (CHO cell, antisense), SEQ ID NOs:2593179-2593525 (CHO cell, antisense), SEQ ID NOs:3155965-3156011 (Gallus, sense), SEQ ID NOs:3156012-3156058 (Gallus, antisense), SEQ ID NOs:315777-3155823 (Canis, sense) and SEQ ID NOs:3155824-3155870 (Canis, antisense), depending on the cultured cell.

Additionally, for example, MAVS expression can be modulated by use of corresponding RNA effector molecule(s) having an antisense strand comprising at least 16 contiguous nucleotides (e.g., at least 17, at least 18, at least 19 nucleotides) of an oligonucleotide nucleotide having a sequence selected from the group consisting of SEQ ID NOs:3156397-3156443 (CHO cell, sense), SEQ ID NOs:3156444-3156490 (CHO cell, antisense), SEQ ID NOs:1607184-1607527 (CHO cell, antisense), SEQ ID NOs:3286682-3286975 (Gallus, sense), SEQ ID NOs:3286976-3287269 (Gallus, antisense), SEQ ID NOs:3286132-3286406 (Canis, sense) and SEQ ID NOs:3286407-3286681 (Canis, antisense), depending on the cultured cell.

There are host cell proteins that impact viral replication in a specific fashion, yet the exact mechanisms for this activity is unresolved. For example, the suppression of the cellular protein casein kinase 2β (CSKN2B) increases influenza replication, protein production and viral titer. Marjuki et al., 3 J. Mol. Signal. 13 (2008). CSKN2B expression can be modulated by use of corresponding RNA effector molecule having an antisense strand comprising at least 16 contiguous nucleotides (e.g., at least 17, at least 18, at least 19 nucleotides) of an oligonucleotide nucleotide having a sequence selected from the group consisting of SEQ ID NOs:2634978-2635358 (CHO cell, antisense), SEQ ID NOs:3289552-3289846 (Gallus, sense), SEQ ID NOs:3289847-3290141 (Gallus, antisense), SEQ ID NOs:3288368-3288657 (Canis, sense), SEQ ID NOs:3288658-3288947 (Canis, antisense), depending on the cultured cell.

A composition, in alternative embodiments, can comprise one or more RNA effector molecules capable of modulating expression of one or multiple genes relating to a common biological process or property of the cell, for example the interferon signaling pathway including IFN, STAT proteins or other proteins in the JAK-STAT signaling pathway, IFNRA1 and/or IFNRA2. For example, viral infection results in swift innate response in infected cells against potential lytic infection, transformation and/or apoptosis, which is characterized by the production of IFNα and IFNβ. This signaling results in activation of IFN-stimulated genes (ISGs) that mediate the effects of IFN. IFN regulatory factor (IRFs) are family of nine cellular factors that bind to consensus IFN-stimulated response elements (ISREs) and induce other ISGs. See Kirshner et al., 79 J. Virol. 9320-24 (2005). The IFNs increase the expression of intrinsic proteins including TRIM5α, Fv, Mx1, eIF2α and 2′-5′ OAS, and induce apoptosis of virus-infected cells and cellular resistance to viral infection. Koyam et al., 43 Cytokine 336-41 (2008). Hence, a particular embodiment provides for a RNA effector molecule that targets a IFNRA1 gene. Other embodiments target one or more genes in the IFN signaling pathway.

Inhibition of IFN signaling responses can be determined by measuring the phosphorylated state of components of the IFN pathway following viral infection, e.g., IRF-3, which is phosphorylated in response to viral dsRNA. In response to type I IFN, Jak1 kinase and TyK2 kinase, subunits of the IFN receptor, STAT1, and STAT2 are rapidly tyrosine phosphorylated. Thus, in order to determine whether the RNA effector molecule inhibits IFN responses, cells can be contacted with the RNA effector molecule, and following viral infection, the cells are lysed. IFN pathway components, such as Jak1 kinase or TyK2 kinase, are immunoprecipitated from the infected cell lysates, using specific polyclonal sera or antibodies, and the tyrosine phosphorylated state of the kinase determined by immunoblot assays with an anti-phosphotyrosine antibody. See, e.g., Krishnan et al., 247 Eur. J. Biochem. 298-305 (1997). A decreased phosphorylated state of any of the components of the IFN pathway following infection with the virus indicates decreased IFN responses by the virus in response to the RNA effector molecule(s).

Efficacy of IFN signaling inhibition can also be determined by measuring the ability to bind specific DNA sequences or the translocation of transcription factors induced in response to viral infection, and RNA effector molecule treatment, e.g., targeting IRF3, STAT1, STAT2, etc. In particular, STAT 1 and STAT2 are phosphorylated and translocated from the cytoplasm to the nucleus in response to type I IFN. The ability to bind specific DNA sequences or the translocation of transcription factors can be measured by techniques known to skilled artisan, e.g., electromobility gel shift assays, cell staining, etc. Another approach to measuring inhibition of IFN induction determines whether an extract from the cell culture producing the desired viral product and contacted with a RNA effector molecule is capable of conferring protective activity against viral infection. More specifically, for example, cells are infected with the desired virus and contacted with a RNA effector. Approximately 15 to 20 hours post-infection, the cells or cell media are harvested and assayed for viral titer, or by quantitative product-enhanced reverse transcriptase (PERT) assay, immune assays, or in vivo challenge.

Host Cell Receptors

In some embodiments, the target gene is a host cell gene (endogenous) that encodes or is involved in the synthesis or regulation of a membrane receptor or other moiety. Modulating expression of the cell membrane can increase or decrease viral infection (e.g., by increasing or decreasing receptor expression), or can increase recovery of product that would otherwise adsorb to host cell membrane (by decreasing receptor expression).

For example, many viruses adhere to host cell-surface heparin, including PCV (Misinzo et al., 80 J. Virol. 3487-94 (2006); CMV (Compton et al., 193 Virology 834-41 (1993)); pseudorabies virus (Mettenleiter et al., 64 J. Virol. 278-86 (1990)); BHV-1 (Okazaki et al., 181 Virology 666-70 (1991)); swine vesicular disease virus (Escribano-Romero et al., 85 Gen. Virol. 653-63 (2004)); and HSV (WuDunn & Spear, 63 J. Virol. 52-58 (1989)). Additionally, enveloped viruses having infectivity associated with surface heparin binding include HIV-1 (Mondor et al., 72 J. Virol. 3623-34 (1998)); AAV-2 (Summerford & Samulski, 72 J. Virol. 1438-45 (1998)); equine arteritis virus (Asagoe et al., 59 J. Vet. Med. Sci. 727-28 (1997)); Venezuelan equine encephalitis virus (Bernard et al., 276 Virology 93-103 (2000)); Sindbis virus (Byrnes & Griffin, 72 J. Virol. 7349-56 (1998); Chung et al., 72 J. Virol. 1577-85 (1998)); swine fever virus (Hulst et al., 75 J. Virol. 9585-95 (2001)); porcine reproductive and respiratory syndrome virus (Jusa et al., 62 Res. Vet. Sci. 261-64 (1997)); and RSV (Krusat & Streckert, 142 Arch. Virol. 1247-54 (1997)). A number of non-enveloped virus associate with cell surface heparin as well. Some picornaviridae family members associate with cell-surface heparin, including, foot-and-mouth disease virus (FMDV) (binds in in vitro culture) (Fry et al., 18 EMBO J. 543-54 (1999); Jackson et al., 70 J. Virol. 5282-87 (1996)); coxsackie virus B3 (CVB3) (Zautner et al., 77 J. Virol. 10071-77 (2003)); Theiler's murine encephalomyelitis virus (Reddi & Lipton, 76 J. Virol. 8400-07 (2002)); and certain echovirus serotypes (Goodfellow et al., 75 J. Virol. 4918-21 (2001)).

Hence, in particular embodiments of the present invention, cellular expression of heparin can be modulated in order to decrease or increase viral adsorption to the host cell. For example, one or more RNA effector molecule(s) can target one or more genes associated with heparin synthesis or structure, such as epimerases, xylosyltransferases, galactosyltransferases, N-acetylglucosaminyl transferases, glucuronosyltransferases, or 2-O-sulfotransferases. See, e.g., Rostand & Esko, 65 Infect. Immun. 1-8 (1997).

In the instance where the expression of cell-surface heparin is increased, a RNA effector molecule can target genes associated with heparin degradation, such as genes encoding heparanase (hep) (e.g., mouse hep GeneID: 15442, mouse hep 2 GeneID: 545291, rat hep GeneID: 64537, rat hep 2 GeneID: 368128, human HEP GeneID: 10855, human HEP 2 GeneID: 60495, Xenopus hep GeneID: 100145320, wild pig Sus scrofa hep GeneID: 100271932, Gallus hep GeneID: 373981, Gallus hep 2 GeneID: 423834, dog hep GeneID: 608707, bovine hep GeneID: 8284471, Callithrix monkey hep GeneID: 100402671, Callithrix hep 2 GeneID: 100407598, P. troglodytes hep GeneID: 461206, rabbit hep GeneID: 100101601, Rhesus Macaque hep GeneID: 707583, or zebrafish hep GeneID: 563020). See Gingis-Velitski et al., 279 J. Biol. Chem. 44084-92 (2004).

Similarly, the infectivity of influenza virus is dependent on the presence of sialic acid on the cell surface (Pedroso et al., 1236 Biochim. Biophys. Acta 323-30 (1995), as is the infectivity of rotaviruses (Is a et al., 23 Glycoconjugate J. 27-37 (2006); Fukudome et al., 172 Virol. 196-205 (1989)), other reoviruses (Paul et al., 172 Virol. 382-85 (1989)), and bovine coronaviruses (Schulze & Herrler, 73 J. Gen. Virol. 901-06 (1992)). Additional host cell-surface receptors include VCAM1 for encephalomyocarditis virus (Huberm 68 J. Virol. 3453-58 (1994); integrin VLA-2 for Echovirus (Bergelson et al., 1718-20 (1992); and members of the immunoglobulin super-family for poliovirus (Mendelson et al., 56 Cell 855-65 (1989). As such, a RNA effector targeting a host sialidase gene can be used to modulate host cell infectivity.

Thus, in some embodiments the gene target includes a host cell gene involved in sialidase (see Wang et al., 10 BMC Genomics 512 (2009)). For example, because influenza binds to cell surface sialic acid residues, decreased sialidase can increase the rate of purification. Target genes include, for example, NEU2 sialidase 2 (cytosolic sialidase) (Gallus Neu2, GeneID: 430542); NEU3 sialidase 3 (membrane sialidase) (Gallus Neu3, GeneID: 68823); solute carrier family 35 (CMP-sialic acid transporter) member A1 (Slc35A1). Example RNA effector molecules targeting SCL35A1 can have the sequences provided in SEQ ID NOs:3154345-3154368 (Gallus, sense) and SEQ ID NOs:3154369-3154392 (Gallus, antisense); and for SCL35A2, SEQ ID NOs:464674-465055 (CHO cell, antisense). For UDP-N-acetylglucosamine 2-epimerase/N-acetylmannosamine kinase (Gne), example siRNAs include SEQ ID NOs:2073971-2074368 (CHO cell, antisense), SEQ ID NOs:3154297-3154320 (Gallus, sense) and SEQ ID NOs:3154321-3154344 (Gallus, antisense)); cytidine monophospho-N-acetylneuraminic acid synthetase (Cmas), example siRNAs shown in SEQ ID NOs:1633101-1633406 (CHO cell, antisense), SEQ ID NOs:3154249-3154272 (Gallus, sense) and SEQ ID NOs:3154273-3154296 (Gallus, antisense)); UDP-Gal:βGlcNAc β1,4-galactosyltransferase (B4GalT1), example siRNAs having sequences chosen from SEQ ID NOs:2528454-2528763 (CHO cell, antisense), SEQ ID NOs:3154153-3154176 (Gallus, sense) and SEQ ID NOs:3154177-3154200 (Gallus, antisense)); and UDP-Gal:βGlcNAc β1,4-galactosyltransferase, polypeptide 6 (B4GalT6), example siRNAs in SEQ ID NOs:1635173-1635561 (CHO cell, antisense), SEQ ID NOs:3154201-3154224 (Gallus, sense) and SEQ ID NOs:3154225-3154248 (Gallus, antisense).

Host Cell Viability

In some embodiments, the production of an immunogenic agent in a host cell is enhanced by introducing into the cell an additional RNA effector molecule that affects cell growth, cell division, cell viability, apoptosis, nutrient handling, and/or other properties related to cell growth and/or division within the cell. The target gene can also encode a host cell protein that directly or indirectly affects one or more aspects of the production of the immunogenic agent. Examples of target genes that affect the production of polypeptides include genes encoding proteins involved in the secretion, folding or post-translational modification of polypeptides (e.g., glycosylation, deamidation, disulfide bond formation, methionine oxidation, or pyroglutamation); genes encoding proteins that influence a property or phenotype of the host cell (e.g., growth, viability, cellular pH, cell cycle progression, apoptosis, carbon metabolism or transport, lactate formation, susceptibility to viral infection or RNAi uptake, activity or efficacy); and genes encoding proteins that impair the production of an immunogenic agent by the host cell (e.g., a protein that binds or co-purifies with the immunogenic agent).

In some embodiments of the invention, the target gene encodes a host cell protein that indirectly affects the production of an immunogenic agent such that inhibiting expression of the target gene enhances production of the immunogenic agent. For example, the target gene can encode an abundantly expressed host cell protein that does not influence directly production of the immunogenic agent, but indirectly decreases its production, for example by utilizing cellular resources that could otherwise enhance production of the immunogenic agent.

In some embodiments, Ago1 (Eukaryotic translation initiation factor 2C, 1); BLK (B lymphoid tyrosine kinase); CCNB3 (Cyclin B3); HILI (piwi-like 2 (Drosophila); HIWI1 (piwi-like 2 (Drosophila); HIWI2 (piwi-like 2 (Drosophila); HIWI3 (piwi-like 2 (Drosophila); is targeted using the methods and compositions described herein.

For optimal production of an immunogenic agent in cell-based bioprocesses described herein, it is desirable to maximize cell viability. Accordingly, in one embodiment, production of an immunogenic agent is enhanced by modulating expression of a cell protein that affects apoptosis or cell viability, such as Bax (BCL2-associated X protein), for example; Bak (BCL2-antagonist/killer 1) (e.g., Gallus Bak, GeneID: 419912), LDHA (lactate dehydrogenase A) (e.g., Gallus LdhA, GeneID: 396221), LDHB (e.g., Gallus LdhB, GeneID: 373997), BIK; BAD (SEQ ID NOs:3049436-3049721), BID (SEQ ID NOs:2582517-2582823), BIM, HRK, BCLG, HR, NOXA, PUMA (SEQ ID NOs:1712045-1712425), BOK (BCL2-related ovarian killer) (e.g., Mus musculus Bok, GeneID: 395445, Gallus Bok, GeneID: 995445, human BOK, GeneID: 666), BOO, BCLB, CASP2 (apoptosis-related cysteine peptidase 2) (e.g., Gallus Casp2, GeneID: 395857) (SEQ ID NOs:2718675-2719039), CASP3 (apoptosis-related cysteine peptidase) (e.g., Gallus Casp3, GeneID: 395476) (SEQ ID NOs:1924836-1925195), CASP6 (e.g., Gallus Casp6, GeneID: 395477 (SEQ ID NOs:2408466-2408843); CASP7 (e.g., Gallus, GeneID: 423901 (SEQ ID NOs:2301618-2301960); CASP8 (e.g., Gallus Casp8, GeneID: 395284, human CASP8 GeneID:841, M. musculus Casp8, GeneID: 12370, Canis Casp8, GeneID:488473) (SEQ ID NOs:2995593-2995870); CASP9 (e.g., Gallus Casp9, GeneID: 426970) (SEQ ID NOs:1412589-1412860), CASP10 (e.g., Gallus Casp10, GeneID: 424081), BCL2 (B-cell CLL/lymphoma 2) (e.g., Gallus Bcl2, GeneID: 396282), p53 (e.g., Gallus p53, GeneID: 396200) (SEQ ID NOs:1283506-1283867), APAF1, HSP70 (e.g., Gallus Hsp70, GeneID: 423504) (SEQ ID NOs:3147029-3147080); TRAIL (TRAIL-LIKE TNF-related apoptosis inducing ligand-like) (e.g., Gallus Trail, GeneID: 395283), BCL2L1 (BCL2-like 1) (e.g., Gallus Bcl2L₁, GeneID: 373954) BCL2L13 (BCL2-like 13 [apoptosis facilitator]) (e.g., Gallus Bcl2113, GeneID: 418163, human BCL2L₁₃, GeneID: 23786), BCL2L14 (BCL2-like 14 [apoptosis facilitator]) (e.g., allus Bcl2114, GeneID: 419096), FASLG (Fas ligand [TNF superfamily, member 6]) (e.g., Gallus Faslg, GeneID: 429064), DPF2 (D4, zinc and double PHD fingers family 2) (e.g., Gallus Dpf2, GeneID: 429064), AIFM2 (apoptosis-inducing factor mitochondrion-associated 2) (e.g., human AIFM2, GeneID: 84883, Gallus Aifm2, GeneID: 423720), AIFM3 (e.g., Gallus Aifm3, GeneID: 416999), STK17A (serine/threonine kinase 17a [apoptosis-inducing]) (e.g., Gallus Stk17A, GeneID: 420775), APITD1 (apoptosis-inducing, TAF9-like domain 1) (e.g., Gallus Apitd1, GeneID: 771417), SIVA1 (apoptosis-inducing factor) (e.g., Gallus Siva1, GeneID: 423493), FAS (TNF receptor superfamily member 6) (e.g., Gallus Fas, GeneID: 395274), TGFβ2 (transforming growth factor β2) (e.g., Gallus TgfB2, GeneID: 421352), TGFBR1 (transforming growth factor, β receptor I) (e.g., Gallus TgfR1, GeneID: 374094), LOC378902 (death domain-containing tumor necrosis factor receptor superfamily member 23) (Gallus GeneID: 378902), or BCL2A1 (BCL2-related protein A1) (e.g., Gallus Bcl2A1, GeneID: 395673). For example, the BAK protein is known to down-regulate cell apoptosis pathways. Suyama et al., S1 Nucl. Acids. Res. 207-08 (2001).

For example, LDHA expression can be modulated by use of a corresponding RNA effector molecule comprising an antisense strand comprising at least 16 contiguous nucleotides (e.g., at least 17, at least 18, at least 19 nucleotides) of the nucleotides in SEQ ID NOs:3154553-3154578 (Gallus, sense), SEQ ID NOs:3154579-3154604 (Gallus, antisense), SEQ ID NOs:3152540-3152603 (CHO cell), SEQ ID NOs:3152843-3152823 (CHO cell), SEQ ID NOs:1297283-1297604 (CHO cell, antisense), SEQ ID NOs:3155589-3155635 (Canis, sense), SEQ ID NOs:3154971-3155018 (Canis, antisense).

Further, for example, the Bak protein is known to down-regulate cell apoptosis pathways. Thus, RNA effector molecules that target Bak can be used to suppress apoptosis and increase product yield, and can comprise an antisense strand comprising at least 16 contiguous nucleotides (e.g., at least 17, at least 18, at least 19 nucleotides) of the nucleotides in SEQ ID NOs:3152412-3152475 (CHO cell), SEQ ID NOs:3152804-3152813), SEQ ID NOs:2259855-220161 (CHO cell, antisense), SEQ ID NOs:3154393-3154413 (Gallus, sense), SEQ ID NOs:3154414-3154434 (Gallus, antisense), SEQ ID NOs:3154827-3154874 (Canis, sense), SEQ ID NOs:3154875-3154922 (Canis, antisense). See also Suyama et al., 51 Nucl. Acids. Res. 207-08 (2001). A particular embodiment thus provides for a RNA effector molecule that targets the Bak gene. A particular embodiment thus provides for a RNA effector molecule that targets the BAK1 gene.

Similarly, Bax protein is known to down-regulate cell apoptosis pathways. Thus, RNA effector molecules that target chicken Bax can be used to suppress apoptosis and increase immunogen product yield, and can comprise an antisense strand comprising at least 16 contiguous nucleotides (e.g., at least 17, at least 18, at least 19 nucleotides) of the nucleotides in SEQ ID NOs:3154393-3154413 (Gallus, sense), SEQ ID NOs:315414-3154434 (Gallus, antisense), SEQ ID NOs:3152412-3152539 (CHO cell), SEQ ID NOs:3152794-3152803 (CHO cell), SEQ ID NOs:3023234-3023515 (CHO cell, antisense), SEQ ID NOs:3154923-3154970 (Canis, sense), and SEQ ID NOs:3154971-3155018 (Canis, antisense).

In some embodiments, administration of RNA effector molecule/s targeting at least one gene involved in apoptosis (e.g., Bak, Bax, caspases etc.) is followed by a administration of glucose to the cell culture medium in order to increase cell density and switch cells to a lactate utilization mode. In some embodiments the concentration of glucose is increased at least 2-fold, at least 3-fold, at least 4 fold, or at least 5-fold.

Another embodiment provides for a plurality of different RNA effector molecules is contacted with the cells in culture to permit modulation of Bax, Bak and LDH expression. In another embodiment, RNA effector molecules targeting Bax and Bak are co-administered to a cell culture during production of the immunogenic agent and can optionally contain at least one additional RNA effector molecule or agent.

Alternatively, one can administer one RNA effector molecule at a time to the cell culture. In this manner, one can easily tailor the average percent inhibition desired for each target gene by altering the frequency of administration of a particular RNA effector molecule. For example, >80% inhibition of lactate dehydrogenase (LDH) may not always be necessary to significantly improve production of an immunogenic agent and under some conditions may even be detrimental to cell viability. Thus, one may desire to contact a cell with a RNA effector molecule targeting LDH at a lower frequency (e.g., less often) than the frequency of contacting with the other RNA effector molecules (e.g., Bax/Bak). Alternatively, the cell can be contacted with a RNA effector molecule targeting LDH at a lower dosage (e.g., lower multiples over the IC₅₀) than the dosage for other RNA effector molecules (e.g., Bax/Bak). For ease of use and to prevent potential contamination it may be preferred to administer a cocktail of different RNA effector molecules, thereby reducing the number of doses required and minimizing the chance of introducing a contaminant to the cell culture.

The production of an immunogenic agent in cell-based bioprocesses described herein can also be optimized by targeting genes that have been identified through screens. These include, for example, PUSL1 (pseudouridylate synthase-like 1) (CHO-Pusl1: SEQ ID NO:3157237; siRNA SEQ ID NOs:3249217-3249316); TPST1 (tyrosylprotein sulfotransferase 1) (e.g., Gallus Tpst1, GeneID: 417546) (CHO TPST1: SEQ ID NO:2613, corresponding siRNAs: SEQ ID NOs:858808-859104), and WDR33 (WD repeat domain 33) (e.g., Gallus Wdr33, GeneID: 424753) (CHO: SEQ ID NO:3433, corresponding siRNAs: SEQ ID NOs:1138341-1138649) (Brass et al., 139 Cell 1243-54 (2009)); Nod2 (nucleotide-binding oligomerization domain containing 2) (CHO: SEQ ID NO:6858; siRNA SEQ ID NOs:2322123-2322429) (Sabbah et al., 10 Nat. Immunol. 1973-80 (2009)); MCT4 (solute carrier family 16, member 4 [monocarboxylic acid transporter 4]) (e.g., Gallus Mct4, GeneID: 395383), ACRC (acidic repeat containing) (e.g., Gallus AcrC, GeneID:422202), AMELY, ATCAY (cerebellar, Cayman type [caytaxin]) (e.g., Gallus Atcay, GeneID: 420094), ANP32B (acidic [leucine-rich] nuclear phosphoprotein 32 family member) (e.g., Gallus Anp32B, GeneID: 420087), DEFA3, DHRS10, DOCK4 (dedicator of cytokinesis 4) (e.g., Gallus Dock4, GeneID: 417779), FAM106A, FKBP1B (FK506 binding protein 1B) (e.g., human FKCB1B, GeneID: 2281, M. musculus Fkbp1b, GeneID: 14226, Gallus Fkbp1B, GeneID: 395254), IRF3, KBTBD8 (kelch repeat and BTB [POZ] domain containing 8) (e.g., Gallus Kbtbd8, GeneID: 416085), KIAA0753 (e.g., Gallus Kiaa0753, GeneID: 417681), LPGAT1 (lysophosphatidyl-glycerol acyltransferase 1) (e.g., Gallus Lpgat1, GeneID: 421375), MSMB (microseminoprotein β) (e.g., Gallus Msmb, GeneID: 423773), NFS1 (nitrogen fixation 1 homolog) (e.g., Gallus Nfs1, GeneID: 419133), NPIP, NPM3 (nucleophosmin/nucleoplasmin 3) (e.g., Gallus Npm3, GeneID: 770430), SCGB2A1, SERPINB7, SLC16A4 (solute carrier family 16, member 4 [monocarboxylic acid transporter 5]) (e.g., Gallus S1c16a4, GeneID: 419809), SPTBN4 (spectrin, β, non-erythrocytic 4) (e.g., Gallus SptBn4, GeneID: 430775), or TMEM146 (Krishnan et al., 2008).

Other target genes that can be affected to optimize immunogen production include genes associated with cell cycle and/or cell proliferation, such as CDKN1B (cyclin-dependent kinase inhibitor 1B, p27, kip1) (e.g., Gallus Cdkn1b, GeneID: 374106), a target for which a siRNA against p27kip1 induces proliferation (Kikuchi et al., 47 Invest. Opthalmol. 4803-09 (2006)); or FOX01, a target for which a siRNA induces aortic endothelial cell proliferation (Fosbrink et al., J. Biol. Chem. 19009-18 (2006). Thus, for example, in CEF or other chicken cells, the expression of CDKN2A, associated with cell division, can be modulated using a corresponding RNA effector molecule having an antisense strand comprising at least 16 contiguous nucleotides (e.g., at least 17, at least 18, at least 19 nucleotides) of an oligonucleotide nucleotide having a sequence selected from the group consisting of SEQ ID NOs:3154663-3154696 (Gallus, sense) and SEQ ID NOs:3154697-3154730 (Gallus, antisense).

Reactive oxygen species (ROS) are toxic to host cells and can mediate non-specific oxidation, degradation and/or cleavage and other structural modifications of the immunogenic agent that lead to increased heterogeneity, decreased biological activity, lower recoveries, and/or other impairments to of biologics produced by methods provided herein. Accordingly, production of an immunogenic agent is enhanced by modulating expression of a pro-oxidant enzyme, such as a CHO cell protein selected from the group consisting of: NAD(p)H oxidase, peroxidase such as a glutathione peroxidase (e.g., glutathione peroxidase 1, glutathione peroxidase 4, glutathione peroxidase 8 (putative), glutathione peroxidase 3, encoded by the oligonucleotides of SEQ ID NO:7213, NO:7582, NO:8011, and NO:9756, respectively (RNA effector molecules: SEQ ID NOs:2439217-2439612, NOs:2560559-2560895, NOs:2703865-2704225, NOs:3151589-3151685, respectively), myeloperoxidase, constitutive neuronal nitric oxide synthase (cnNOS), xanthine oxidase (XO) (SEQ ID NOs:374846-375216) and myeloperoxidase (MPO), 15-lipoxygenase-1 (SEQ ID NOs:2480018-2480362), NADPH cytochrome c reductase, NAPH cytochrome c reductase, NADH cytochrome b5 reductase (SEQ ID NOs:569460-569777, NOs:1261910-1262218, NOs:2195311-2195681, NOs:3146048-3146071, NOs:259827-260060, respectively), and cytochrome P4502E1.

Additionally, protein production can be enhanced by modulating expression of a protein that affects the cell cycle of host cells (e.g., CHO cells) such as a cyclin (e.g., cyclin M4, cyclin J, cyclin T2, cyclin-dependent kinase inhibitor 1A (P21), cyclin-dependent kinase inhibitor 1B, cyclin M3, cyclin-dependent kinase inhibitor 2B (p15, inhibits CDK4), cyclin E2, S100 calcium-binding protein A6 (calcyclin), cyclin-dependent kinase 5, regulatory subunit 1 (p35), cyclin T1, inhibitor of CDK, cyclin A1 interacting protein 1, by use of corresponding a RNA effector molecule comprising an an antisense strand comprising at least 16 contiguous nucleotides (e.g., at least 17, at least 18, at least 19 nucleotides) of an oligonucleotide nucleotide having a sequence selected from the group consisting of SEQ ID NOs:2447340-2447632, NOs:2463782-2464073, NOs:2466004-2466274, NOs:2659502-2659871, NOs:2731076-2731440, NOs:2748583-2748914, NOs:2895015 2895359, NOs:2904183-2904530, NOs:2966362-2966657, NOs:3088848-3089061, NOs:3107706-3107919, and NOs:3122589-3122734, respectively), or a cyclin dependent kinase (CDK). In some embodiments, the cyclin-dependent kinase is a CHO cell cyclin-dependent kinase selected from the group consisting of: CDK2 (SEQ ID NOs:1193336-1193684), CDK4 (SEQ ID NOs:1609522-1609852), P10 (SEQ ID NOs:3013998-3014274), P21 (SEQ ID NOs:2659502-2659871), P27 (SEQ ID NOs:2731076-2731440), p53, P57, p161NK4a, P14ARF, and CDK4 (SEQ ID NOs:1609522-1609852). For example, in various embodiments, the expression of one or more proteins that affect cell cycle progression can be transiently modulated during the growth and/or production phases of heterologous protein production in order to enhance expression and recovery of heterologous proteins.

In addition, production of excess ammonia in bioprocessing is a common problem in large scale cell culture. High ammonia concentrations result in reduced cell and product yields, depending on cell line and process conditions. Liberation of ammonia is thought to occur through the breakdown of glutamine to glutamate by glutaminase, and/or through the conversion of glutamate to a-ketoglutarate by glutamate dehydrogenase. Therefore, in one embodiment, biologics production can be enhanced by modulating expression of a protein that affects ammonia production, such as glutaminase or glutamate dehydrogenase. A particular embodiment provides for a RNA effector that targets CHO cell glutaminase having the transcript of SEQ ID NO:311 (CHO311.1). In one embodiment the RNA effector is a siRNA selected from SEQ ID NOs:105170-105438, which target glutaminase. In another embodiment, the RNA effector targets CHO cell glutamate dehydrogenase having SEQ ID NO:569 (CHO569.1). In one embodiment the RNA effector is a siRNA selected from SEQ ID NOs:177779-178010, which target CHO cell glutamate dehydrogenase 1.

It is known that production of lactic acid in cell cultures inhibits cell growth and influences metabolic pathways involved in glycolysis and glutaminolysis (Lao & Toth, 13 Biotech. Prog., 688-91 (1997)). The accumulation of lactate in cells is caused mainly by the incomplete oxidation of glucose to CO₂ and H₂O, in which most of the glucose is oxidized to pyruvate and finally converted to lactate by lactate dehydrogenase (LDH). The accumulation of lactic acid in cells is detrimental to achieving high cell density and viability. Accordingly, in one embodiment, immunogenic protein production is enhanced by modulating expression of a protein that affects lactate formation, such as lactate dehydrogenase A (LDHA). Hence, a particular embodiment provides for a RNA effector molecule that targets the LDHA 1 gene.

In some embodiments, glucose utilization of cells is manipulated by modulation expression of e.g., target genes Myc and AKT. In one embodiment the target gene is CHO myelocytomatosis oncogene comprising the sequence of SEQ ID NO:2185 (CHO2185.1). In one embodiment the RNA effector molecule is a siRNA having a sequence selected from SEQ ID NOs:713438-713745. In one embodiment the RNA effector molecule is a siRNA having a sequence selected from SEQ ID NOs:713438-713473. In one embodiment the target gene is CHO thymoma viral proto-oncogene-1 comprising the nucleotides of SEQ ID NO:1793 (CHO1793.1). In one embodiment the RNA effector molecule is a siRNA having a sequence selected from SEQ ID NOs:581286-581643. In one embodiment the RNA effector molecule is a siRNA having a sequence selected from SEQ ID NOs:581286-581334.

In one embodiment, a cell culture is treated as described herein with RNA effector molecules that permit modulation of Bax, Bak and LDH expression. In another embodiment, the RNA effector molecules targeting Bax, Bak and LDH can be administered in combination with one or more additional RNA effector molecules and/or agents. Provided herein is a cocktail of RNA effector molecules targeting Bax, Bak and LDH expression, which can optionally be combined with additional RNA effector molecules or other bioactive agents as described herein.

In some embodiments, production of an immunogenic agent is enhanced by modulating expression of a protein that affects cellular pH, such as LDH or lysosomal V-type ATPase.

In some embodiments, production of an immunogenic agent is enhanced by modulating expression of a protein that affects carbon metabolism or transport, such as GLUT1, for example, by contacting the cell with a RNA effector molecule wherein the RNA effector molecule comprises an antisense strand comprising at least 16 contiguous nucleotides (e.g., at least 17, at least 18, at least 19 nucleotides) of an oligonucleotide having the nucleotide sequence selected from the group consisting of SEQ ID NOs:438155-438490, GLUT2, GLUT3, GLUT4, PTEN (SEQ ID. NOs:6091-6940) (with a RNA effector molecule wherein the RNA effector molecule comprises an antisense strand comprising at least 16 contiguous nucleotides (e.g., at least 17, at least 18, at least 19 nucleotides) of the nucleotide sequence selected from the group consisting of SEQ ID NOs:69091-69404 (CHO cell, antisense), or LDH (with a RNA effector molecule wherein the RNA effector molecule comprises an antisense strand comprising at least 16 contiguous nucleotides (e.g., at least 17, at least 18, at least 19 nucleotides) of the oligonucleotide having a nucleotide sequence selected from the group consisting of SEQ ID NOs:1297283-1297604)—see also Table 10 with LDHs).

TABLE 4
GLUTS and PTEN

| SEQ ID | Transcript | | | Avg | siRNA SEQ |
|---|---|---|---|---|---|
| NO: | No. | consL | Description | Coverage | ID NOs: |
| 1375 | CHO1375.1 | 2298 | solute carrier family 2 (facilitated | 14.092 | 438155-438490 |
| | | | glucose transporter), member 1 | | |
| 6869 | CHO6869.1 | 910 | solute carrier family 2, (facilitated | 0.818 | 2325698-2325997 |
| | | | glucose transporter), member 8 | | |
| 7909 | CHO7909.1 | 656 | solute carrier family 2 (facilitated | 0.689 | 2669929-2670303 |
| | | | glucose transporter), member 13 | | |
| 189 | CHO189.1 | 3384 | PTEN (phosphatase and | 0.633 | 69091-69404 |
| | | | tensin homolog) | | |

In some embodiments, production of an immunogenic agent is enhanced by modulating expression of cofilin (for example a muscle cofilin 2, or non-muscle cofilin-1). In one embodiment, a cell with a RNA effector molecule wherein the RNA effector molecule comprises an antisense strand comprising at least 16 contiguous nucleotides (e.g., at least 17, at least 18, at least 19 nucleotides) of the oligonucleotide having a nucleotide sequence selected from the group consisting of SEQ ID NOs:435213-435610, targeting the CHO muscle cofilin 2 (SEQ ID NO:1366). In another embodiment, a cell with a RNA effector molecule wherein the RNA effector molecule comprises an antisense strand comprising at least 16 contiguous nucleotides (e.g., at least 17, at least 18, at least 19 nucleotides) of the oligonucleotide having a nucleotide sequence selected from the group consisting of SEQ ID NOs:1914036-1914356, targeting the CHO non-muscle cofilin 1 (SEQ ID NO:5716).

In some embodiments, production of an immunogenic agent is enhanced by modulating expression of a protein that affects uptake or efficacy of a RNA effector molecule in host cells, such as ApoE, Mannose/GalNAc-receptor, and Eri1. In various embodiments, the expression of one or more proteins that affects RNAi uptake or efficacy in cells is modulated according to a method provided herein concurrently with modulation of one or more additional target genes, such as a target gene described herein, in order to enhance the degree and/or extent of modulation of the one or more additional target genes.

In some embodiments, the production of an immunogenic agent is enhanced by inducing a stress response in the host cells which causes growth arrest and increased productivity. A stress response can be induced, e.g., by limiting nutrient availability, increasing solute concentrations, or low temperature or pH shift, and oxidative stress. Along with increased productivity, stress responses can also have adverse effects on protein folding and secretion. In some embodiments, such adverse effects are ameliorated by modulating the expression of a target gene encoding a stress response protein, such as a protein that affects protein folding and/or secretion described herein.

In some embodiments, production of an immunogenic agent is enhanced by modulating expression of a protein that affects cytoskeletal structure, e.g. altering the equilibrium between monomeric and filamentous actin. In one embodiment the target gene encodes cofilin and a RNA effector molecule inhibits expression of cofilin. In one embodiment, at least one RNA effector molecule increases expression of a target gene selected from the group consisting of: cytoplasmic actin capping protein (CapZ), Ezrin (VIL2), and Laminin A. See, e.g., Table 5, as follows:

TABLE 5
Example hamster genes and siRNAs (antisense strand) targeting Laminin and CapZ

| SEQ | | | Avg | siRNA SEQ |
|---|---|---|---|---|
| ID NO: | consL | Description | Cov | ID NOs: |
| 763 | 2614 | capping protein (actin filament) muscle Z-line, α 1 | 5.404 | 235917-236159 |
| 3104 | 1768 | capping protein (actin filament) muscle Z-line, α 2 | 15.011 | 1026343-1026702 |
| 3590 | 1647 | capping protein (actin filament) muscle Z-line, β | 60.716 | 1190654-1190998 |
| 5752 | 1156 | capping protein (actin filament), gelsolin-like | 62.723 | 1927144-1927507 |
| 1081 | 2436 | ezrin | 31.498 | 339220-339540 |
| 122 | 3653 | laminin, α 5 | 10.318 | 48814-49139 |
| 8777 | 444 | laminin, α 2 | 0.046 | 2954307-2954650 |
| 3157936 | 2200 | laminin, α 3 | 0.41 | 3160721-3160820 |

The modulation of expression (e.g., inhibition) of a target gene by a RNA effector molecule can be further alleviated by introducing a second RNA effector molecule, wherein at least a portion of the second RNA effector molecule is complementary to a target gene encoding a protein that mediates RNAi in the host cell. For example, the modulation of expression of a target gene can be alleviated by introducing into the cell a RNA effector molecule that inhibits expression of an Argonaute protein (e.g., argonaute-2) or other component of the RNAi pathway of the cell. In one embodiment, the immunogenic agent is transiently inhibited by contacting the cell with a first RNA effector molecule targeted to the immunogenic agent. The inhibition of expression of the immunogenic agent is then alleviated by introducing into the cell a second RNA effector molecule targeted against a gene encoding a protein of the RNAi pathway.

Additionally, the production of a desired immunogenic agent can be enhanced by introducing into the cell a RNA effector molecule during the production phase to modulate expression of a target gene encoding a protein that affects protein expression, post-translational modification, folding, secretion, and/or other processes related to production and/or recovery of the desired immunogenic agent. Alternatively, the production of an immunogenic agent is enhanced by introducing into the cell a RNA effector molecule which inhibits cell growth and/or cell division during the production phase.

Post-Translational Processing

Post-translational modifications can require additional bioprocess steps to separate modified and unmodified polypeptides, increasing costs and reducing efficiency of biologics production. Accordingly, in some embodiments, in production of a polypeptide agent in a cell is enhanced by modulating the expression of a target gene encoding a protein that affects post-translational modification. In additional embodiments, biologics production is enhanced by modulating the expression of a first target gene encoding a protein that affects a first post-translational modification, and modulating the expression of a second target gene encoding a protein that affects a second post-translational modification.

More specifically, proteins expressed in eukaryotic cells can undergo several post-translational modifications that can impair production and/or the structure, biological activity, stability, homogeneity, and/or other properties of the immunogenic agent. Many of these modifications occur spontaneously during cell growth and polypeptide expression and can occur at several sites, including the peptide backbone, the amino acid side-chains, and the amino and/or carboxyl termini of a given polypeptide. In addition, a given polypeptide can comprise several different types of modifications. For example, proteins expressed in avian and mammalian cells can be subject to acetylation, acylation, ADP-ribosylation, amidation, ubiquitination, methionine oxidation, disulfide bond formation, methylation, demethylation, sulfation, formation of cysteine, formation of pyroglutamate, formylation, gamma-carboxylation, hydroxylation, iodination, myristoylation, oxidation, proteolytic processing, phosphorylation, prenylation, racemization, glycosylation, gluconoylation, sequence mutations, N-terminal glutamine cyclization and deamidation, and asparagine deamidation. N-terminal asparagine deamidation can be reduced by contacting the cell with a RNA effector molecule targeting the N-terminal Asn amidase (encoded, for example, by SEQ ID NO:5950), wherein the RNA effector molecule comprises an antisense strand comprising at least 16 contiguous nucleotides (e.g., at least 17, at least 18, at least 19 nucleotides) of the oligonucleotide having a nucleotide sequence selected from the group consisting of SEQ ID NOs:1999410-1999756.

In some embodiments, immunogen production is enhanced by modulating expression of a target gene which encodes a protein involved in protein deamidation. Proteins can be deaminated via several pathways, including the cyclization and deamidation of N-terminal glutamine and deamidation of asparagine. Thus, in one embodiment, the protein involved in protein deamidation is N-terminal asparagine amidohydrolase. Protein deamidation can lead to altered structural properties, reduced potency, reduced biological activity, reduced efficacy, increased immunogenicity, and/or other undesirable properties and can be measured by several methods, including but not limited to, separations of proteins based on charge by, e.g., ion exchange chromatography, HPLC, isoelectric focusing, capillary electrophoresis, native gel electrophoresis, reversed-phase chromatography, hydrophobic interaction chromatography, affinity chromatography, mass spectrometry, or the use of L-isoaspartyl methyltransferase.

When the immunogenic agent comprises a glycoprotein, such as a viral product having viral surface membrane proteins or monoclonal antibody having glycosylated amino acid residues, biologics production can be enhanced by modulating expression of a target gene that encodes a protein involved in protein glycosylation. Glycosylation patterns are often important determinants of the structure and function of mammalian glycoproteins, and can influence the solubility, thermal stability, protease resistance, antigenicity, immunogenicity, serum half-life, stability, and biological activity of glycoproteins.

In various embodiments, the protein that affects glycosylation is selected from the group consisting of: dolichyl-diphosphooligosaccharide-protein glycosyltransferase (SEQ ID NOs:2742894-2743239), UDP glycosyltransferase, UDP-Gal:βGlcNAc beta 1,4-galactosyltransferase (SEQ ID NOs:851115-851489, NOs:1552461-1552728, NOs:1562813-1563108, and NOs:1635173-1635561), UDP-galactose-ceramide galactosyltransferase, fucosyltransferase (SEQ ID NOs:209841-210227), protein O-fucosyltransferase (SEQ ID NOs:916726-917035), N-acetylgalactosaminytransferase (SEQ ID NOs:57147-57422, NOs:65737-65999, NOs:1013002-1013376, NOs:1363583-1363970, NOs:1546609-1546999, NOs:1965217-1965613, NOs:2876241-2876595), particularly T4 (SEQ ID NOs:2876241-2876595), O-GlcNAc transferase (SEQ ID NOs:607012-607348), oligosaccharyl transferase (SEQ ID NOs:89738-90024, NOs:262368-262621), O-linked N-acetylglucosamine transferase, and α-galactosidase (SEQ ID NOs:1600968-1601288) and β-galactosidase (SEQ ID NOs:690601-690989).

In other embodiments. The protein that affects glycosylation is selected, for example, from Table 6, as follows:

TABLE 6
O-linked glycosylation

| SEQ | | | Avg | siRNA SEQ |
|---|---|---|---|---|
| ID NO: | consL | Description | Cov | ID NOs: |
| 150 | 3549 | UDP-N-acetyl--D-galactosamine:polypeptide | 11.757 | 57147-57422 |
| | | N-acetylgalactosaminyltransferase 1 | | |
| 178 | 3411 | UDP-N-acetyl--D-galactosamine:polypeptide | 22.835 | 65737-65999 |
| | | N-acetylgalactosaminyltransferase 2 | | |
| 1720 | 2167 | protein-O-mannosyltransferase 2 | 1.099 | 555946-556293 |
| 1869 | 2123 | O-linked N-acetylglucosamine (GlcNAc) transferase | 0.839 | 607012-607348 |
| | | (UDP-N-acetylglucosamine:polypeptide-N- | | |
| | | acetylglucosaminyl transferase) | | |
| 3065 | 1776 | UDP-N-acetyl--D-galactosamine:polypeptide | 1.546 | 1013002-1013376 |
| | | N-acetylgalactosaminyltransferase 10 | | |
| 4007 | 1548 | protein-O-mannosyltransferase 1 | 1.418 | 1331135-1331436 |
| 4654 | 1402 | UDP-N-acetyl--D-galactosamine: polypeptide | 0.782 | 1546609-1546999 |
| | | N-acetylgalactosaminyltransferase 7 | | |
| 5740 | 1158 | protein O-linked mannose β1,2-N- | 2.323 | 1922712-1923111 |
| | | acetylglucosaminyltransferase | | |
| 6857 | 913 | protein O-fucosyltransferase 1 | 0.441 | 2321807-2322122 |
| 258 | 3197 | STT3, subunit of the oligosaccharyltransferase | 25.073 | 89738-90024 |
| | | complex, homolog B (S. cerevisiae) | | |
| 1114 | 2420 | ribophorin II | 272.65 | 350422-350752 |
| 2417 | 1954 | mannoside acetylglucosaminyltransferase 2 | 5.098 | 792371-792746 |
| 2614 | 1903 | dolichyl-di-phosphooligosaccharide- | 179.1 | 859105-859389 |
| | | protein glycotransferase | | |
| 4441 | 1452 | dolichyl pyrophosphate phosphatase 1 | 2.663 | 1476398-1476763 |
| 4945 | 1339 | mannoside acetylglucosaminyltransferase 5 | 0.5 | 1645857-1646201 |
| 5594 | 1191 | mannoside acetylglucosaminyltransferase 1 | 3.072 | 1870192-1870557 |
| 5740 | 1158 | protein O-linked mannose β1,2-N- | 2.323 | 1922712-1923111 |
| | | acetylglucosaminyltransferase | | |
| 8007 | 632 | asparagine-linked glycosylation 6 homolog | 1.15 | 2702432-2702775 |
| | | (yeast, α-1,3,-glucosyltransferase) | | |
| 8404 | 518 | keratinocyte associated protein 2 | 6.913 | 2832647-2833030 |

In further embodiments, production of an immunogenic glycoprotein is enhanced by modulating expression of a sialidase or a sialytransferase enzyme. Terminal sialic acid residues of glycoproteins are particularly important determinants of glycoprotein solubility, thermal stability, resistance to protease attack, antigenicity, and specific activity. For example, when terminal sialic acid is removed from serum glycoproteins, the desilylated proteins have significantly decreased biological activity and lower circulatory half-lives relative to sialylated counterparts. The amount of sialic acid in a glycoprotein is the result of two opposing processes, i.e., the intracellular addition of sialic acid by sialyltransferases and the removal of sialic acid by sialidases. Thus, in some embodiments, production of a glycoprotein is enhanced by inhibiting expression of a sialidase and/or activating expression of a sialytransferase. Example sialyltransferase targets and exemplary siRNAs are found in Table 7, as follows:

TABLE 7
Example sialyltransferase targets

| SEQ | | | Avg | siRNA SEQ |
|---|---|---|---|---|
| ID NO: | consL | Description | Cov | ID NOs: |
| 2088 | 2048 | ST3 β-galactoside α-2,3-sialyltransferase 1 | 5.651 | 681105-681454 |
| 2167 | 2021 | ST3 β-galactoside α-2,3-sialyltransferase 4 | 13.01 | 707535-707870 |
| 3411 | 1689 | ST3 β-galactoside α-2,3-sialyltransferase 3 | 3.964 | 1131123-1131445 |
| 3484 | 1672 | ST3 β-galactoside α-2,3-sialyltransferase 5 | 21.148 | 1155324-1155711 |
| 4186 | 1504 | ST6 (α-N-acetyl-neuraminyl-2,3-β-galactosyl- | 5.237 | 1391079-1391449 |
| | | 1,3)-N-acetylgalactosaminide α-2,6- | | |
| | | sialyltransferase 6 | | |
| 4319 | 1476 | ST3 β-galactoside α-2,3-sialyltransferase 2 | 1.043 | 1435989-1436317 |
| 3157960 | 2282 | ST8 α-N-acetyl-neuraminide α-2,8- | 1.629 | 3246817-3246916 |
| | | sialyltransferase 4 | | |
| 3158211 | 343 | ST6 (α-N-acetyl-neuraminyl-2,3-β-galactosyl- | 0.282 | 3260605-3260704 |
| | | 1,3)-N-acetylgalactosaminide α-2,6- | | |
| | | sialyltransferase 4 | | |

In some embodiments, immunogenic agent production is enhanced by modulating expression of a glutaminyl cyclase which catalyzes the intramolecular cyclization of N-terminal glutamine residues into pyroglutamic acid, liberating ammonia (pyroglutamation). Glutaminyl cyclase modulation can be accomplished by contacting the cell with a RNA effector molecule targeting the glutaminyl cyclase gene (for example, hamster glutaminyl cyclase encoded by SEQ ID NO:5486), wherein the RNA effector molecule comprises an antisense strand comprising at least 16 contiguous nucleotides (e.g., at least 17, at least 18, at least 19 nucleotides) of the oligonucleotide having a nucleotide sequence selected from the group consisting of SEQ ID NOs:1832626-1832993.

In some embodiments, production of immunogenic agents containing disulfide bonds is enhanced by modulating expression of a protein that affects disulfide bond oxidation, reduction, and/or isomerization, such as protein disulfide isomerase or sulfhydryl oxidase. Disulfide bond formation can be particularly problematic for the production of multi-subunit proteins or peptides in eukaryotic cell culture. Examples of multi-subunit proteins or peptides include receptors, extracellular matrix proteins, immunomodulators, such as MHC proteins, full chain antibodies and antibody fragments, enzymes and membrane proteins.

In some embodiments, protein production is enhanced by modulating expression of a protein that affects methionine oxidation. Reactive oxygen species (ROS) can oxidize methionine (Met) to methionine sulfoxide (MetO), resulting in increased degradation and product heterogeneity, and reduced biological activity and stability. In some embodiments, the target gene encodes a methionine sulfoxide reductase, which catalyzes the reduction of MetO residues back to methionine. For example, wherein the CHO cell RNA effector molecule comprises an antisense strand comprising at least 16 contiguous nucleotides (e.g., at least 17, at least 18, at least 19 nucleotides) of the oligonucleotide having a nucleotide sequence selected from the group consisting of SEQ ID NOs:2044387-2044676, SEQ ID NOs:2557492-2557809, and SEQ ID NOs:3076104-3076309.

Immunogenic agents (including some live attenuated viruses) produced in cell culture on an industrial-scale are typically secreted by cultured cells and recovered and purified from the surrounding cell culture media. In general, the rate of protein production and the yield of recovered protein is directly related to the rate of protein folding and secretion by the host cells. For example, an accumulation of misfolded proteins in the endoplasmic reticulum (ER) of host cells can slow or stop secretion via the unfolded protein response (UPR) pathway. The UPR is triggered by stress-sensing proteins in the ER membrane which detect excess unfolded proteins. UPR activation leads to the upregulation of chaperone proteins (e.g., Bip) which bind to misfolded proteins and facilitate proper folding. UPR activation also upregulates the transcription factors XBP-1 (e.g., CHO cell SEQ ID NOs:187955-188152) and CHOP (e.g., CHO cell SEQ ID NOs:2813622-2813956). CHOP generally functions as a negative regulator of cell growth, differentiation and survival, and its upregulation via the UPR causes cell cycle arrest and increases the rate of protein folding and secretion to clear excess unfolded proteins from the cell. Hence, cell cycle can be promoted initially, then repressed during virus production phase to increase viral product yield. An increase the rate of immunogenic protein secretion by the host cells can be measured by, e.g., monitoring the amount of protein present in the culture media over time.

The present invention provides methods for enhancing the production of a secreted polypeptide in cultured eukaryotic host cells by modulating expression of a target gene which encodes a protein that affects protein secretion by the host cells. In some embodiments, the target gene encodes a protein of the UPR pathway, such as IRE1, PERK, ATF4 (CHO cell, SEQ ID NOs:1552067-1552460), ATF6 (CHO cell, SEQ ID NOs:570138-570498), eIF2a (CHO cell, SEQ ID NOs:1828122-1828492), GRP78 (CHO cell, SEQ ID NOs:292590-292837), GRP94 (CHO cell, SEQ ID NOs:180574-180954), calreticulin (CHO cell, SEQ ID NOs:895691-896051) or a variant thereof, or a protein that regulates the UPR pathway, such as a transcriptional control element (e.g., the cis-acting UPR element (UPRE)).

Other target genes involved in protein secretion are listed in Table 8, which identifies example hamster transcript target genes and exemplary siRNAs (antisense strand):

TABLE 8
Example Chinese hamster secretory pathway targets

| SEQ | | | Avg | |
|---|---|---|---|---|
| ID NO: | consL | Description | Cov | siRNA SEQ ID NOs: |
| 8 | 4838 | myosin VA | 2.412 | 12025-12278 |
| 584 | 2751 | transmembrane emp24-like trafficking | 22.212 | 182087-182337 |
| | | protein 10 (yeast) | | |
| 1448 | 2267 | glycyl-tRNA synthetase | 58.453 | 462911-463286 |
| 2119 | 2036 | ADP-ribosylation factor interacting protein 1 | 1.425 | 691369-691690 |
| 2236 | 2001 | MON1 homolog A (yeast) | 8.293 | 730977-731347 |
| 2859 | 1843 | retinoid X receptor | 3.715 | 942750-943051 |
| 3432 | 1685 | lipase maturation factor 1 | 6.857 | 1138015-1138340 |
| 4066 | 1533 | WD repeat domain 77 | 15.26 | 1350827-1351146 |
| 4826 | 1363 | N-acetylglucosamine-1-phosphate | 0.701 | 1605188-1605495 |
| | | transferase, and β subunits | | |
| 5380 | 1240 | K intermediate/small conductance Ca- | 8.029 | 1795510-1795838 |
| | | activated channel, subfamily N, member 4 | | |
| 5799 | 1146 | lysosomal trafficking regulator | 0.206 | 1944185-1944541 |
| 7480 | 768 | endoplasmic reticulum protein 29 | 24.355 | 2526951-2527343 |
| 8119 | 595 | serglycin | 9.946 | 2738723-2739031 |
| 3157722 | 251 | forkhead box A1 | 0.147 | 3261005-3261104 |

In some embodiments, the protein that affects protein secretion is a molecular chaperone selected from the group consisting of: Hsp40 (e.g., CHO cell SEQ ID NOs:677203-677558), HSP47 (e.g., CHO cell SEQ ID NOs:777036-777317), HSP60 (e.g., CHO cell SEQ ID NOs: 494743-495086), Hsp70 (e.g., CHO cell SEQ ID NOs:3147029-3147080), HSP90, HSP100, protein disulfide isomerase (e.g., CHO cell SEQ ID NOs:72748-72996), peptidyl prolyl isomerase (e.g., CHO cell SEQ ID NOs:38781-39067, NOs:1074139-1074475, NOs:1127061-1127426, NOs:1649170-1649515, NOs:2197146-2197532, NOs:2253978-2254373, NOs:2261765-2262058, NOs:2275330-2275633, NOs:2579547-2579908, and NOs:3115010-3115199), calnexin (e.g., CHO cell SEQ ID NOs:61559-61785), Erp57 (e.g., CHO cell SEQ ID NOs:774355-774677), and BAG-1.

In some embodiments, the protein that affects protein secretion is selected from the group consisting of: gamma-secretase, p115, a signal recognition particle (SRP) protein, secretin, and a kinase (e.g., MEK).

The production of immunogenic agents in cell culture can be negatively affected by proteins which have an affinity for the immunogenic agent or a molecule or factor that binds specifically to the immunogenic agent. For example, a number of heterologous proteins have been shown to bind the glycoproteins heparin and heparan sulfate at host cell surfaces. This can lead to the co-purification of heparin, heparan sulfate, and/or heparin/heparan sulfate-binding proteins with recombinant protein products, decreasing yield and reducing homogeneity, stability, biological activity, and/or other properties of the recovered proteins. Examples of heterologous proteins which have been shown to bind heparin and/or heparan sulfate include BMP3 (bone morphogenetic protein 3 or osteogenin), TNF-α, GDNF, TGF-β family members, and HGF. Therefore, in one embodiment, the production of a heterologous protein, such as BMP3, TNF-α, GDNF, TGF-β family members, or HGF, or another immunogenic agent in cultured host cells is enhanced by contacting the cells with a RNA effector molecule which modulates (e.g., inhibits) expression and/or production of heparin and/or heparan sulfate. In one embodiment, the level of heparin and/or heparan sulfate is reduced by modulating expression of a host cell enzyme involved in the production of heparin and/or heparan sulfate, such as a host cell xylotransferase (SEQ ID NOs:1554774-1555054).

In some embodiments, for example when in immunogenic agent is a viral particle, such as an influenza virus, target genes can include those involved in reducing sialic acid from the host cell surface, which reduces virus binding, and therefore increases recovery of the virus in cell culture media (i.e., less virus remains stuck on host cell membranes). These targets include: solute carrier family 35 (CMP-sialic acid transporter) member A1 (SLC35A1) (e.g., CHO gene inferred from M. muscuslus Slac35a1, GeneID:24060) (Gallus target gene sequences selected from SEQ ID NOs:3154345-3154368 and NOs:3154369-3154392) (CHO cell target gene sequences selected from SEQ ID NOs:464674-465055), solute carrier family 35 (UDP-galactose transporter), member A2 (SLC35A2) (e.g., CHO gene inferred from M. muscuslus Slc35a2, GeneID: 22232) UDP-N-acetylglucosamine 2-epimerase/N-acetylmannosamine kinase (GNE) (e.g., CHO gene inferred from M. muscuslus Gne, GeneID: 10090) (Gallus target gene sequences selected from SEQ ID NOs:3154297-3154320 and NOs:3154321-3154344) (CHO cell target gene sequences selected from SEQ ID NOs:2073971-2074368), cytidine monophospho-N-acetylneuraminic acid synthetase (Cmas) (e.g., CHO gene inferred from M. muscuslus Cmas, GeneID: 12764) (Gallus target gene sequences selected from SEQ ID NOs:3154249-3154272 and NOs:3154273-3154296) (CHO cell target gene sequences selected from SEQ ID NOs:1633101-1633406), UDP-Gal:βGlcNAc β1,4-galactosyltransferase (B4GalT1) (e.g., CHO gene inferred from M. muscuslus B4galT1, GeneID: 14595) (Gallus target gene sequences selected from SEQ ID NOs:3154153-3154176 and NOs:3154177-3154200) (CHO cell target gene sequences selected from SEQ ID NOs:2528454-2528763), and UDP-Gal:βGlcNAc β1,4-galactosyltransferase, polypeptide 6 (B4GalT6) (e.g., CHO gene inferred from M. muscuslus B4GalT6, GeneID: 56386) (Gallus target gene sequences selected from SEQ ID NOs:3154201-3154224 and NOs:3154225-3154248) (CHO cell target gene sequences selected from SEQ ID NOs:1635173-1635561).

Additional targets can include those involved in avian host sialidase (see Wang et al., 10 BMC Genomics 512 (2009)), because influenza binds to cell surface sialic acid residues, thus decreased sialidase can increase the rate of infection or purification: NEU2 sialidase 2 (cytosolic sialidase) (e.g., Gallus Neu2, GeneID: 430542) and NEU3 sialidase 3 (membrane sialidase) (e.g., Gallus Neu3, GeneID: 68823). Additional target genes include miRNA antagonists that can be used to determine if this is the basis of some viruses not growing well in cells, for example Dicer (dicer 1, ribonuclease type III) because knock-down of Dicer leads to a modest increase in the rate of infection (Matskevich et al., 88 J. Gen. Virol. 2627-35 (2007)); or ISRE (interferon-stimulated response element), as a decoy titrate TFs away from ISRE-containing promoters. Example genes and targets associated with sialidases (neuraminidases) are shown in Table 9, as follows:

TABLE 9
Example sialidases (neuraminidase)

| | | | Avg | siRNA |
|---|---|---|---|---|
| SEQ ID NO: | consL | Description | Coverage | SEQ ID NOs: |
| 4150 | 1513 | neuraminidase 1 | 11.083 | 1378888-1379212 |
| 4816 | 1365 | neuraminidase 2 | 6.612 | 1601657-1601952 |
| 7787 | 692 | neuraminidase 3 | 0.275 | 2628786-2629181 |

The use of bioprocesses for the manufacture of immunogenic agents at an industrial scale is often confounded by the presence of pathogens, such as active viral particles, and other adventitious agents (e.g., prions), often necessitating the use of expensive and time consuming steps for their detection, removal (e.g., viral filtration) and/or inactivation (e.g., heat treatment) to conform to regulatory procedures. Such problems can be exacerbated due to the difficulty in detecting and monitoring the presence of such viruses. Accordingly, in some embodiments, methods are provided for enhancing production of an immunogenic agent by modulating expression of a target gene affecting the susceptibility of a host cell to pathogenic infection. For example, in some embodiments, the target gene is a host cell protein that mediates viral infectivity, such as the transmembrane proteins XPR1 (e.g., CHO cell SEQ ID NOs:62021-62362), RDR, Flvcr, CCR5, CXCR4, CD4, Pit1, and Pit2 (e.g., CHO cell SEQ ID NOs:3068222-3068455).

Although a target sequence is generally 10 to 30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with a RNA effector molecule agent, mediate the best inhibition of target gene expression. Thus, although the sequences identified herein represent effective target sequences, it is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified herein, further optimization could be achieved by systematically either adding or removing nucleotides to generate longer or shorter sequences and testing those and sequences generated by walking a window of the longer or shorter size up or down the target RNA from that point. Coupling this approach to generating new candidate targets with testing for effectiveness of RNA effector molecules based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

### III. BIOCONTAMINATION

Cell lines used commonly in biotechnology manufacturing processes, such as CHO cells, have been demonstrated to produce retrovirus-like particles. Moreover, MMV (murine minute virus) contamination in a large-scale biologics manufacturing process has occurred, and was attributed to adventitious contamination of raw materials used in production. Consequently, international regulatory agencies require biologics manufacturers to employ a comprehensive viral clearance strategy, including characterization of cell lines and raw materials, employing robust viral inactivation and removal steps, and testing of process intermediates and final products. Multiple orthogonal steps, including chromatographic methods, physiochemical inactivation (e.g., low pH, solvent detergent), and size exclusion-based filtration, together yield cumulative inactivation and removal of viruses. See, e.g., Marques et al., 25 Biotech. Prog. 483-91 (2009); Khan et al., 52 Biotech. Appl. Biochem. 293-301 (2009). Viral clearance and clearance validation are some of the most time-consuming and revenue-eating activities in bioprocessing: Downstream processing accounts for about 70% of the total biomanufacturing cost. Chochois et al., 36 Bioprocess Intl. (June, 2009). Downstream bioprocessing filter products, alone, cost biotechnology and vaccine makers more than $1 billion annually.

Thus, in further embodiments, production is enhanced by introducing into the cell a RNA effector molecule that inhibits expression of viral proteins in host cells. More specifically, for example, latent DNA viruses (such as herpesviruses) and endogenous retroviruses (ERVs), or retroviral elements are likely present in all vertebrates. Endogenous retroviral sequences are an integral part of eukaryotic genomes, and although the majority of these sequences are defective, some can produce infectious virus, either spontaneously or upon long-term culture. ERV virus production can also be induced upon treatment with various chemical or other agents that can be part of the normal production system. Additionally, although many endogenous retroviruses do not readily re-infect their own cells, they can infect other species in vitro and in vivo. For example, two of three subgroups of pig ERVs (PERVs), can infect human cells in vitro.

There are at least twenty-six distinct groups of human endogenous retroviruses (HERVs); and bird, mouse, cat, and pig harbor replication-competent ERVs that are capable of interacting with related exogenous virus. Retrovirus-induced tumorigenesis can involve the generation of a novel pathogenic virus by recombination between replication-competent and -defective sequences and/or activation of a cellular oncogene by a long terminal repeat (LTR) due to upstream or downstream insertion of retrovirus sequences. Thus, the activation of an endogenous, infectious retrovirus in a cell substrate that is used for the production of biologics is an important safety concern, especially in the case of live, viral vaccines, where minimal purification and inactivation steps are used in order to preserve high vaccine potency.

Adventitious viruses represent a major risk associated with the use of cell-substrate derived biologicals, including vaccines for human use. The possibility for viral contamination exists in primary cultures and established cultures, as well as Master Cell Banks, end-of-production cells, and bulk harvest fluids. For example, this is a major obstacle to the use of neoplastic-immortalized cells for which the mechanism of transformation is unknown, because these could have a higher risk of containing oncogenic viruses. Extensive testing for the presence of potential extraneous agents is therefore required to ensure the safety of the vaccines. The most common scenarios for adventitious viral contamination of biologics include bovine viral diarrhoea virus in foetal bovine serum; porcine parvovirus in porcine substrates; and murine minute virus, reovirus, vesivirus and Cache Valley virus in CHO cell-derived bulk harvests. The three last-named viral entities are believed to be introduced via bovine serum used during the manufacturing process (during scale-up or during the entire process).

During the production of live attenuated viral vaccines, removal of contaminating viral particles, nucleic acid, or proteins is problematic because any antiviral approach must leave the viral product intact and immunogenic. Indeed, endogenous avian viral particles have been found in commercially released human measles and mumps vaccines derived from chicken embryo fibroblasts. Moreover, endogenous viral proteins, particularly envelop proteins, often inhibit the efficiency of recombinant viral vectors used in creating transformed cell lines. Further, endogenous virus can aggravate the immune response of the host cell, often triggered during viral production or retroviral transduction. Hence, there remains a need for techniques that inhibit adventitious, latent and endogenous viral activity, and thus increase purity and yield of immunogenic agents produced in cells.

The present invention provides for enhancing production of an immunogenic agent by introducing into the cell a RNA effector molecule to modulate expression of a target gene, optionally encoding a protein, that is involved with the expression of an adventitious, latent or endogenous virus. Thus, in some embodiments, the production of an immunogenic agent in a host cell is enhanced by introducing into the cell a RNA effector molecule that inhibits expression of a latent or endogenous viral protein such that the infectivity and/or load of the desired immunogenic agent in the cell is increased.

For example, a particular advantage of cell-culture based inactivated influenza virus or influenza viral antigens is the absence of egg-specific proteins that might trigger an allergic reaction against egg proteins. Therefore, the use according to the invention is especially suitable for the prophylaxis of influenza virus infections, particularly in populations that constitute higher-risk groups, such as asthmatics, those with allergies, and also people with suppressed immune systems and the elderly.

The cultivation conditions under which a virus strain is grown in cell culture also are of great significance with respect to achieving an acceptably high yield of the strain. In order to maximize the yield of a desired virus strain, both the host system and the cultivation conditions must be adapted specifically to provide an environment that is advantageous for the production of a desired virus strain. Many viruses are restricted to very specific host systems, some of which are very inefficient with regard to virus yields. Some of the mammalian cells which are used as viral host systems produce virus at high yields, but the tumorigenic nature of such cells invokes regulatory constraints against their use for vaccine production.

The problems arising from the use of serum in cell culture and/or protein additives derived from an animal or human source added to the culture medium, e.g., the varying quality and composition of different batches and the risk of contamination with mycoplasma, viruses or BSE-agent, are well-known. In general, serum or serum-derived substances like albumin, transferrin or insulin can contain unwanted agents that can contaminate the culture and the immunogenic agents produced therefrom. Furthermore, human serum derived additives have to be tested for all known viruses, like hepatitis or HIV, which can be transmitted by serum. Bovine serum and products derived therefrom, for example trypsin, bear the risk of bovine spongiform encephalitis-contamination. In addition, all serum-derived products can be contaminated by still unknown agents. Therefore, cells and culture conditions that do not require serum or other serum derived compounds are being pursued.

For example, the production of smallpox vaccine, modified vaccinia virus Ankara (MVA) is amplified in cell cultures of primary or secondary chicken embryo fibroblasts (CEF). The CEF are obtained from embryos of chicken eggs that have been incubated for 10 to 12 days, from which the cells are then dissociated and purified. These primary CEF cells can either be used directly or after one further cell passage as secondary CEF cells. Subsequently, the primary or secondary CEF cells are infected with the MVA. For the amplification of MVA the infected cells are incubated for 2 to 3 days at 37° C. See, e.g., Meyer et al., 72 J. Gen. Virol. 1031-38 (1991); Sutter et al., 12 Vaccine 1032-40 (1994). Many pox viruses replicate efficiently in CEF incubated at temperatures below 37° C., such as 30° C. See U.S. Pat. No. 6,924,137.

The use of established mammalian cell lines, such as Madin-Darby canine kidney (MDCK) line, has been successful in replicating some viral strains. Nevertheless, a number of virus strains will not replicate in the MDCK line. In addition, fears over possible adverse effects associated with employing cells with a tumorigenic potential for human vaccine production have precluded the use of MDCK, a highly transformed cell line, in this context.

Other attempts at developing alternative vaccine production methods have been undertaken. U.S. Pat. No. 4,783,411 discusses a method for preparing influenza vaccines in goldfish cell cultures. The virus particles for infecting the goldfish cell cultures, after their establishment, were obtained from chicken embryo cultures or from infected CD-I strain mice. The virus is passaged at least twice in the goldfish cell cultures, resulting in an attenuated influenza virus which can be used as a live vaccine. Additionally, African green monkey kidney epithelial cells (Vero) and chicken embryo cells (CEC) have been adapted to grow and produce influenza virus and recombinant influenza proteins in serum-free, protein-free media. See WO 96/015231.

Although the use of protein and serum free media limits the risk from adventitious virus contamination, it does not address the continued risk posed by latent viruses or endogenous retroviruses that exist in cell banks. The activation of an endogenous, infectious retrovirus in a cell substrate that is used for the production of biologics is an important safety concern, especially in the case of live, viral vaccines, where there are minimal purification and inactivation steps in order to preserve high vaccine potency.

In some embodiments, an RNA effector molecule targeting a vesivirus can be used with the methods and compositions described herein. Exemplary RNA effector molecules that target vesivirus are include, but are not limited to, those in Table 63 below:

TABLE 63
Duplexes targeting vesivirus with
modified nucleotides

| Duplex | Sense/ | |
|---|---|---|
| No | Antisense | Sequence |
| 1 | S | cuGuGGcAAGAcuAcucuudTsdT |
| | AS | AAGAGuAGUCUUGCcAcAGdTsdT |
| 2 | S | ccuAcAcAGGcAAcGAGGudTsdT |
| | AS | ACCUCGUUGCCUGUGuAGGdTsdT |
| 3 | S | GAAucAAAuuucAcAGAAudTsdT |
| | AS | AUUCUGUGAAAUUUGAUUCdTsdT |
| 4 | S | GAGuuGcGAccuGuGGAuAdTsdT |
| | AS | uAUCcAcAGGUCGcAACUCdTsdT |
| 5 | S | cAAGuGGGAuucAAcucAAdTsdT |
| | AS | UUGAGUUGAAUCCcACUUGdTsdT |
| 6 | S | GGAAcAucuAcGAuuAcAudTsdT |
| | AS | AUGuAAUCGuAGAUGUUCCdTsdT |
| 7 | S | GGcAAGAcuAcucuuGcuudTsdT |
| | AS | AAGcAAGAGuAGUCUUGCCdTsdT |
| 8 | S | cAGGcAAcGAGGuGuGcAudTsdT |
| | AS | AUGcAcACCUCGUUGCCUGdTsdT |
| 9 | S | GuuGAGAuGGuAAAuAcAAdTsdT |
| | AS | UUGuAUUuACcAUCUcAACdTsdT |
| 10 | S | GcuAAGAGAAGAcucAuuudTsdT |
| | AS | AAAUGAGUCUUCUCUuAGCdTsdT |
| 11 | S | cAAccAccAAAcGuAAcAAdTsdT |
| | AS | UUGUuACGUUUGGUGGUUGdTsdT |
| 12 | S | cAuGuucAccuAuGGuGAudTsdT |
| | AS | AUcACcAuAGGUGAAcAUGdTsdT |
| 13 | S | cAAGAcuAcucuuGcuuAudTsdT |
| | AS | AuAAGcAAGAGuAGUCUUGdTsdT |
| 14 | S | GcAucAuuGAuGAAuucGAdTsdT |
| | AS | UCGAAUUcAUcAAUGAUGCdTsdT |
| 15 | S | GGAAAGGuGuucuccuccAdTsdT |
| | AS | UGGAGGAGAAcACCUUUCCdTsdT |
| 16 | S | GAuGuuucuGAuGccAuuAdTsdT |
| | AS | uAAUGGcAUcAGAAAcAUCdTsdT |
| 17 | S | GcuGuuGcuAcGcuuucuudTsdT |
| | AS | AAGAAAGCGuAGcAAcAGCdTsdT |
| 18 | S | GuGAuGAuGGcGuGuAcAudTsdT |
| | AS | AUGuAcACGCcAUcAUcACdTsdT |
| 19 | S | cuAcucuuGcuuAuGccAudTsdT |
| | AS | AUGGcAuAAGcAAGAGuAGdTsdT |
| 20 | S | cGAcucuAAuccGGAAucAdTsdT |
| | AS | UGAUUCCGGAUuAGAGUCGdTsdT |
| 21 | S | ccuccAAAuAcGuGAuuAudTsdT |
| | AS | AuAAUcACGuAUUUGGAGGdTsdT |
| 22 | S | cuGAuGccAuuAuGucuAudTsdT |
| | AS | AuAGAcAuAAUGGcAUcAGdTsdT |
| 23 | S | GGuAuGccAcuAAccucuAdTsdT |
| | AS | uAGAGGUuAGUGGcAuACCdTsdT |
| 24 | S | GcGuGuAcAucGuAccAAAdTsdT |
| | AS | UUUGGuACGAUGuAcACGCdTsdT |
| 25 | S | cuucuGuucucAAucucAAdTsdT |
| | AS | UUGAGAUUGAGAAcAGAAGdTsdT |
| 26 | S | GAcucuAAuccGGAAucAAdTsdT |
| | AS | UUGAUUCCGGAUuAGAGUCdTsdT |
| 27 | S | cAAAuAcGuGAuuAuGAcAdTsdT |
| | AS | UGUcAuAAUcACGuAUUUGdTsdT |
| 28 | S | GcAuGAAuucGGcuucAuudTsdT |
| | AS | AAUGAAGCCGAAUUcAUGCdTsdT |
| 29 | S | cGuGuAcAucGuAccAAAudTsdT |
| | AS | AUUUGGuACGAUGuAcACGdTsdT |
| 30 | S | cuGuucucAAucucAAuAudTsdT |
| | AS | AuAUUGAGAUUGAGAAcAGdTsdT |
| 31 | S | cucuAAuccGGAAucAAAudTsdT |
| | AS | AUUUGAUUCCGGAUuAGAGdTsdT |
| 32 | S | cGuGAuuAuGAcAucAAAudTsdT |
| | AS | AUUUGAUGUcAuAAUcACGdTsdT |
| 33 | S | GuAccGcAAGGGAAuGcAudTsdT |
| | AS | AUGcAUUCCCUUGCGGuACdTsdT |
| 34 | S | cAAccAcuGccucuuAGuudTsdT |
| | AS | AACuAAGAGGcAGUGGUUGdTsdT |
| 35 | S | cuGuuAuGccuAAuGucuudTsdT |
| | AS | AAGAcAUuAGGcAuAAcAGdTsdT |
| 36 | S | cAAuAuuGAccAccAcGAudTsdT |
| | AS | AUCGUGGUGGUcAAuAUUGdTsdT |
| 37 | S | cGGAAucAAAuuucAcAGAdTsdT |
| | AS | UCUGUGAAAUUUGAUUCCGdTsdT |
| 38 | S | GuGAuuAuGAcAucAAAuAdTsdT |
| | AS | uAUUUGAUGUcAuAAUcACdTsdT |
| 39 | S | cAAGGGAAuGcAucGGuAudTsdT |
| | AS | AuACCGAUGcAUUCCCUUGdTsdT |
| 40 | S | GGGuGuGcAcucAuccAAudTsdT |
| | AS | AUUGGAUGAGUGcAcACCCdTsdT |
| 41 | S | cuuucuuccuAuGGAcuAAdTsdT |
| | AS | UuAGUCcAuAGGAAGAAAGdTsdT |
| 42 | S | cAcGAuGccuAcAcAGGcAdTsdT |
| | AS | UGCCUGUGuAGGcAUCGUGdTsdT |
| 43 | S | GGAAucAAAuuucAcAGAAdTsdT |
| | AS | UUCUGUGAAAUUUGAUUCCdTsdT |
| 44 | S | GAuuAuGAcAucAAAuAAudTsdT |
| | AS | AUuAUUUGAUGUcAuAAUCdTsdT |
| 45 | S | GcAucGGuAuuGcGuuGAudTsdT |
| | AS | AUcAACGcAAuACCGAUGCdTsdT |
| 46 | S | GGAGAAGGGuGuuGAuGuudTsdT |
| | AS | AAcAUcAAcACCCUUCUCCdTsdT |
| 47 | S | GcGcuucuuGAcAGAAAuudTsdT |
| | AS | AAUUUCUGUcAAGAAGCGCdTsdT |

Endogenous Retrovirus

Retroviruses replicate by reverse transcription, mediated by a RNA-dependent DNA polymerase (reverse transcriptase), encoded by the viral pol gene. Retroviruses also carry at least two additional genes: the gag gene encodes the proteins of the viral skeleton, matrix, nucleocapsid, and capsid; the env gene encodes the envelope glycoproteins. Additionally, retroviral transcription is regulated by promoter regions or “enhancers” situated in highly repeated regions (LTRs) which are present at both ends of the retroviral genome.

During the infection of a cell, reverse transcriptase makes a DNA copy of the RNA genome; this copy can then integrate into the host cell genome. Retroviruses can infect germ cells or embryos at an early stage and be transmitted by vertical Mendelian transmission. These endogenous retroviruses (ERVs) can degenerate during generations of the host organism and lose their initial properties. Some ERVs conserve all or part of their properties or of the properties of their constituent motifs, or acquire novel functional properties having an advantage for the host organism. These retroviral sequences can also undergo, over the generations, discrete modifications which will be able to trigger some of their potential and generate or promote pathological processes.

Human endogenous retroviral sequences (HERVs) represent a substantial part of the human genome. These retroviral regions exist in several forms: complete endogenous retroviral structures combining gag, pol and env motifs, flanked by repeat nucleic sequences which exhibit a significant analogy with the LTR-gag-pol-env-LTR structure of infectious retroviruses; truncated retroviral sequences, for example the retrotransposons lack their env domain; and the retroposons that lack the env and LTR regions. ERVs capable of shedding virus particles are often called type C ERVs.

Important ERVs include human teratocarcinoma retrovirus (HTDV), or HERV-K, an endogenous retrovirus known to produce viral particles from endogenous provirus. Lower et al., 68 J. Gen. Virol. 2807-15 (1987); Mold et al., 4 J. Biomed. Sci. 78082 (2005). HERV-R is another important ERV, because it has been found to be expressed in many tissues, including the adrenal cortex and various adrenal tumors such as cortical adenomas and pheochromocytomas. Katsumata et al., 66 Pathobiology 209-15 (1998). Murine leukemia virus (MLV) is another important ERV, that produces infective virus particles in rodent-derived cell culture upon induction. Khan & Sears, 106 Devel. Biol. 387-92 (2001). Indeed, cell culture changes that significantly alter the metabolic state of the cells and/or rates of protein expression (e.g., pH, temperature shifts, sodium butyrate addition) measurably increased the rate of endogenous retroviral synthesis in CHO cells. Brorson et al., 80 Biotech. Bioengin. 257-67 (2002).

An on-line database, called HERVd—Human Endogenous Retrovirus Database (NAR Molecular Biology Database Collection entry number 0495), has been compiled from the human genome nucleotide sequences, obtained mostly in the various ongoing Human Genome Projects. This provides a relatively simple and fast environment for screening HERVs, and makes it possible to continuously improve classification and characterization of retroviral families. The HERVd database now contains retroviruses from more than 90% of the human genome. Additionally, ERV sequences can be obtained readily through the National Institutes of Health's on-line “Entrez Gene” site.

Further regarding ERVs, embodiments of the present invention target at least one gene or LTR of primate/human Class I Gamma ERVs pt01-Chr10r-17119458, pt01-Chr5-53871501, BaEV, GaLV, HERV-T, HERV-R (HERV-3, ERV3 env gene, GeneID: 2086), HERV-E (ERVE1, GeneID: 85314), HERV-ADP, HERV-I, MER4like, HERV-FRD (ERVFRD1, Env protein, GeneID: 405754; P. troglodytes Env protein, GeneID: 471856; Rattus norvegicus Herv-frd Env polyprotein, GeneID: 290348), HERV-W (ERVWE2, ERV-W, env(C7), member 2, P. troglodytes, GeneID: 100190905; HERVWEL ERV-W, env(C7), member 1, GeneID: 30816), HERV-H(HHLA1, HERV-H LTR-associating protein 1, GeneID:10086, P. troglodytes GeneID: 736282; Hhla1, mouse GeneID: 654498; HHLA2, HERV-H LTR-associating protein 2, GeneID: 11148; HHLA3, HERV-H LTR-associating protein 3, GeneID: 11147; Xenopus hhla2, GeneID:734131), HERVH-RTVLH2, HERVH-RGH2, HERV-Hconsensus, HERV-Fcl; primate/human Epsilon endogenous retrovirus hg15-chr3-152465283; primate/human Intermediate (epsilon-like) HERVL66; primate/human Class III Spuma-like ERVs HSRV, HFV, HERV-S, HERV-L, HERVL40, HERVL74; primate/human Delta ERV HTLV-1, HTLV-2; primate/human Lenti ERV (lentivirus) HIV-1, HIV-2; primate/human Class II, Beta ERVs MPMV, MMTV, HML1, HML2, HML3, HML4, HML7, HML8, HML5, HML10, HML6, HML9, human teratocarcinoma-derived retrovirus (HTDV/HERV-K), or HERV-V (HERV-V1 Enyl, GeneID: 147664; HERV-V2, HSV2, GeneID: 100271846).

Additional primate ERV genes that can be targeted by the methods of the present invention include LOC471586 (similar to ERV-BabFcenv provirus ancestral Env polyprotein, P. troglodytes GeneID: 471586), LOC470639 (similar to ERV-BabFcenv provirus ancestral Env polyprotein, P. troglodytes GeneID: 470639); LOC100138322 (similar to HERV-K_{—}7p22.1 provirus ancestral Pol protein, Bos taurus GeneID: 10013822; LOC110138431 (similar to HERV-K_{—}1q22 provirus ancestral Pol protein, B. taurus GeneID: 100138431; LOC100137757 (similar to HERV-K_{—}6q14.1 provirus ancestral Gag-Pol polyprotein, B. taurus GeneID: 100137757); LOC100141085 (similar to HERV-K_{—}8p23.1 provirus ancestral Pol protein, B. taurus GeneID: 100141085); LOC100138106 (similar to HERV-F(c)1_Xq21.33 provirus ancestral Gag polyprotein, B. taurus GeneID: LOC100138106); LOC100140731 (similar to HERV-W_{—}3q26.32 provirus ancestral Gag polyprotein B. taurus, GeneID: 100140731); LOC100139657 (similar to HERV-W_{—}3q26.32 provirus ancestral Gag polyprotein B. taurus GeneID: 100139657).

In other embodiments of the present invention, the ERV is rodent Class II, Beta ERV mouse mammary tumor (MMTV, GeneID: 2828729; MMTVgp7, GeneID: 1491863; MMTV env GeneID: 1491862; MMTVgp1, GeneID: 1724724; MMTVgp2, GeneID: 1724723; MMTV pol GeneID: 1491865; MMTV pro, GeneID: 1491865; MMTV gag, GeneID: 1491864); rodent Class I Gamma ERV MLV (Mlv1, mouse GeneID: 108317); feline Class I Gamma ERV FLV; ungulate Class I Gamma ERV PERV; ungulate Delta ERV BLV; ungulate lentivirus Visna, EIAV; ungulate Class II, Beta ERV JSRV; avian Class III, Spuma-like ERVs gg01-chr7-7163462; gg01-chrU-52190725, gg01-Chr4-48130894; avian Alpha ERVs ALV (ALV pol GeneID: 1491910; ALV p2, GeneID: 1491909; ALV p10, GeneID: 1491908; ALV env, GeneID: 1491907; ALV transmembrane protein, tm, GeneID: 1491906; ALV trans-acting factor, GeneID: 1491911), gg01-chr1-15168845; avian Intermediate Beta-like ERVs gg01-chr4-77338201; gg01-ChrU-163504869, gg01-chr7-5733782; Reptilian Intermediate Beta-like ERV Python-molurus; Fish Epsilon ERV WDSV; fish Intermediate (epsilon-like) ERV SnRV; Amphibian Epsilon ERV Xen1; Insect Errantivirus ERV Gypsy; or Tyl in Saccharomyces cerevisiae, yeast ORF161 (ERV-1-like protein, Ectocarpus siliculosus virus 1, GeneID: 920716).

Further regarding ERVs, as noted herein the HERV-K ERVs are particularly relevant because they can be activated by a variety of stimuli. Hence, aspects of the present invention target genes of the HERV-K family, including HERV-K3, GeneID: 2088; HERV-K2, GeneID: 2087; HERV-K_{—}11q22.1 provirus ancestral Pol protein, GeneID: 100133495; HERV-K7, GeneID: 449619; HERV-K6, GeneID: 64006; HERV-K(1), ERVK4, GeneID: 60359; and HERV-K(II), ERVK5, GeneID: 60358; LOC100133495 (HERV-K_{—}11q22.1 provirus ancestral Pol protein, GeneID: 100133495).

As described herein, in particular aspects of the present invention the target gene is an ERV env gene, for example eERV family W, env(C7), member 1 (ERVWE1), GeneID: 30816; LOC147664 (HERV-V 1 or EnvV1), GeneID: 147664; HERV-FRD provirus Env polyprotein (ERVFRDE1), GeneID: 405754 and GeneID: 471856; ERV sequence K, 6 (ERVK6 or HERV-K108), GeneID: 64006; ERV sequence 3 envelope protein (ERV3), GeneID: 2086 and GeneID: 100190893; ALV Env protein, GeneID: 1491907, or the Env protein of HERV-K18.

In a particular embodiment, the expression of HERV-K Enyl can be modulated by use of a corresponding RNA effector molecule having an antisense strand comprising at least 16 contiguous nucleotides (e.g., at least 17, at least 18, at least 19 nucleotides) of an oligonucleotide nucleotide having a sequence selected from the group consisting of SEQ ID NOs:3287270-3287569 (sense) and SEQ ID NOs:3287570-3287869 (antisense).

In addition to targeting ERV genes and regulatory sequences, some embodiments of the present invention target ERV receptors. For example, human solute carrier family 1 (neutral amino acid transporter), member 5 (SLC1A5, GeneID: 6510) is a receptor for Simian type D retrovirus and feline endogenous RD-114 virus. Solute carrier family 1 (glutamate/neutral amino acid transporter), member 4 (Slc1a4, GeneID: 55963) and member 5 (Slc1a5, GeneID: 20514) are mouse versions of related proteins. Human solute carrier family 1 (glutamate/neutral amino acid transporter), member 4 (SLC1A4, GeneID: 6509), is used as receptor by HERV-W Env glycoprotein. Thus, inhibition of cellular viral receptors can decrease receptor interference, latent, endogenous or adventitious viral infection, and thus increase the production of immunogenic agent in the cell.

Latent Virus

Bornaviruses are genus of non-segmented, negative-sense, non-retroviral RNA viruses that establish persistent infection in the cell nucleus. Elements homologous to the bornavirus nucleoprotein (N) gene exist in the genomes of several mammalian species, and produce mRNA that encodes endogenous Borna-like N (EBLN) elements. Horie et al., 463 Nature 84-87 (2010). Hence, in some embodiments of the invention, the target gene is a bornaviral gene.

Latent DNA viruses that can be targeted by the methods of the present invention include adenoviruses. For example, species of C serotype adenovirus can establish latent infection in human tissues. See Garnett et al., 83 J. Virol. 2417-28 (2000). Avian adenovirus and adenovirus-associated virus (AAV) proteins have been produced by specific-pathogen-free chicks, indicating that avian AAV can exist as a latent infection in the germ line of chickens. Sadasiv et al., 33 Avian Dis. 125-33 (1989); see also Katano et al., 36 Biotechniq. 676-80 (2004). In some embodiments of the invention, the target gene is a latent DNA virus. For example, the target gene can be the latent membrane protein (LMP)-2A from HHV-4 (EBV), GeneID: 3783751, which protein also transactivates the Env protein of HERV-K18.

Circoviridae are DNA viruses that exhibit a latent phase. Porcine circoviridae type 1 (PCV 1) was found to have contaminated Vero cell banks from which rotavirus vaccine was made, causing a temporary FDA hold on administration of the vaccine. Assoc. Press, March 23 (2010). The genomes of PCV1 virus are provided herein are PCV1 AY193712.1 (SEQ ID NO:3154148), PCV1 EF533941.1 (SEQ ID NO:3154149), PCV1 FJ475129.2 (SEQ ID NO:3154150), PCV1 GU371908.1 (SEQ ID NO:3154151), and PCV1 GU722334.1 (SEQ ID NO:3154152).

An embodiment of the present invention provides for a RNA effector molecule that inhibits a PCV1 rep or cap gene. The rep gene of PCV1 is indispensable for replication of viral DNA. Mankertz & Hillenbrand, 279 Virol. 429-38 (2001). In a particular embodiment, the expression of PCV 1 Rep protein can be modulated by use of a corresponding RNA effector molecule having an antisense strand comprising at least 16 contiguous nucleotides (e.g., at least 17, at least 18, at least 19 nucleotides) of an oligonucleotide nucleotide having a sequence selected from the group consisting of SEQ ID NOs:3152824-3153485 (sense), SEQ ID NOs:3153486-3154147 (antisense), and the tables provided herein.

In another particular embodiment, the expression of PCV1 Cap protein can be modulated by use of a corresponding RNA effector molecule having an antisense strand comprising at least 16 contiguous nucleotides (e.g., at least 17, at least 18, at least 19 nucleotides) of an oligonucleotide nucleotide having a sequence selected from the group consisting of SEQ ID NOs:3154731-3154778 (sense), SEQ ID NOs:3154778-3154826 (antisense), and the tables provided herein.

Adventitious Virus

As used herein an “adventitious virus” or “adventitious viral agent” refers to a virus contaminant present within a immunogenic agent, including, for example, vaccines, cell lines and other cell-derived products. Regarding vaccine products, for example, exogenous, adventitious ALV was found in commercial Marek's Disease vaccines propagated in CEF or DEF cell cultures by different manufacturers. Moreover, some of these vaccines were also contaminated with endogenous ALV. Fadly et al., 50 Avian Diseases 380-85 (2006); Zavala & Cheng, 50 Avian Diseases 209-15 (2006).

Other embodiments of the present invention target the genes of adventitious animal viruses, including vesivirus, porcine circovirus, lymphocytic choriomeningitis virus, porcine parvovirus, adenoassociated viruses, reoviruses, rabies virus, papillomavirus, herpesviruses, leporipoxviruses, and leukosis virus (ALV), hantaan virus, Marburg virus, SV40, SV20, Semliki Forest virus (SFV), simian virus 5 (sv5), feline sarcoma virus, porcine parvovirus, adenoassociated viruses (AAV), mouse hepatitis virus (MHV), Moloney murine leukemia virus (MoMLV or MMLV, gag protein GeneID: 1491870), murine leukemia virus (MuLV), pneumonia virus of mice (PVM), Theiler's encephalomyelitis virus (THEMV), murine minute virus (MMV or MVM, GeneID: 2828495, vp1, GeneID: 148592; vp, GeneID: 1489591; ns1, GeneID: 1489590), mouse adenovirus (MAV), mouse cytomegalovirus (MCMV), mouse rotavirus (EDIM), Kilham rat virus (KRV), Toolan's H-1 virus, Sendai virus (SeV, also known as murine parainfluenza virus type 1 or hemagglutinating virus of Japan (HVJ)), rat coronavirus (RCV or sialodacryoadenitis virus (SDA)), pseudorabies virus (PRV), Cache Valley virus, bovine diarrhea virus, bovine parainfluenza virus type 3, bovine respiratory syncytial virus, bovine adenoviruses, bovine parvoviruses, bovine herpesvirus 1 (infectious bovine rhinotracheitis virus), other bovine herpesviruses, bovine reovirus, other bovine herpesviruses, bovine reovirus, bluetongue viruses, bovine polyoma virus, bovine circovirus, and orthopoxviruses other than vaccinia, pseudocowpox virus (a widespread parapoxvirus that can infect humans), papillomavirus, herpesviruses, leporipoxviruses, or exogenous retroviruses.

In a particular embodiment, the expression of MMLV Gag protein can be modulated by use of a corresponding RNA effector molecule having an antisense strand comprising at least 16 contiguous nucleotides (e.g., at least 17, at least 18, at least 19 nucleotides) of an oligonucleotide nucleotide having a sequence selected from the group consisting of SEQ ID NOs:3287870-3288118: (sense) and SEQ ID NOs:3288119-3288367 (antisense).

In a particular embodiment, the expression of vesivirus can be modulated by use of a corresponding RNA effector molecule having an antisense strand comprising at least 16 contiguous nucleotides (e.g., at least 17, at least 18, at least 19 nucleotides) of an oligonucleotide nucleotide having a sequence selected from the group consisting of SEQ ID NOs: 3152604-3152713 and the tables provided herein.

Other embodiments target human-origin adventitious agents including HIV-1 and HIV-2; human T cell lymphotropic virus type I (HTLV-I) and HTLV-II; human hepatitis A, B, and C viruses; human cytomegalovirus (CMV); EBV; HHV 6, 7, and 8; human parvovirus B19; reoviruses; polyoma (JC/BK) viruses; SV40 virus; human coronaviruses; human papillomaviruses; influenza A, B, and C viruses; various human enteroviruses; human parainfluenza viruses; and human respiratory syncytial virus.

Parvoviridae are single-stranded DNA viruses with genomes of about 4 to 5 kilobases. This family includes: Dependovirus such as human helper-dependent adeno-associated virus (AAV) serotypes 1 to 8, autonomous avian parvoviruse, and adeno associated viruses (AAV 1-8); Erythrovirus such as bovine, chipmunk, and autonomous primate parvoviruses, including human parvoviruses B19 (the cause of Fifth disease) and V9; and Parvovirus that includes parvoviruses of other animals and rodents, carnivores, and pigs, including MVM. These parvoviruses can infect several cell types and have been described in clinical samples. AAVs, in particular, have been implicated in decreased replication, propagation, and growth of other virus.

MVM gains cell entry by deploying a lipolytic enzyme, phospholipase A2 (PLA2), that is expressed at the N-terminus of virion protein 1 (VP1, also called MMVgp3), the MVM minor coat protein, GeneID: 1489592. Farr et al., 102 PNAS 17148-53 (2005). Other MVM targets can be chosen from MVM VP (also called MMVgp2), GeneID: 1489591; and MVM non-structural, initiator protein (NS1, also called MMVgp1), GeneID: 1489590. In a particular embodiment, the expression of MVM NS2 protein can be modulated by use of a corresponding RNA effector molecule having an antisense strand comprising at least 16 contiguous nucleotides (e.g., at least 17, at least 18, at least 19 nucleotides) of an oligonucleotide nucleotide having a sequence selected from the group consisting of SEQ ID NOs:3285524-3285827 (sense) and SEQ ID NOs:3285828-3286131 (antisense).

Polyomaviruses are double-stranded DNA viruses that can infect, for example, humans, primates, rodents, rabbits, and birds. Polyomaviruses (PyV) include SV40, JC and BK viruses, Murine pneumonotropic virus, hamster PyV, murine PyV virus, and Lymphotropic papovavirus (LPV, the African green monkey papovavirus). The sequences for these viruses are available via GenBank. See also U.S. Patent Pub. No. 2009/0220937. Because of their tumorigenic and oncogenic potential, it is important to eliminate these viruses in cell substrates used for vaccine production.

Papillomaviridae contains more that 150 known species representing varying host-specificity and sequence homology. They have been identified in mammals (humans, simians, bovines, canines, ovines) and in birds. Majority of the human Papillomaviruses (HPVs), including all HPV types traditionally called genital and mucosal HPVs belong to supergroup A. Within supergroup A, there are 11 groups; the most medically important of these are the human Papillomaviruses HPV 16, HPV 18, HPV 31, HPV 45, HPV 11, HPV 6 and HPV 2. Each of these has been reported as “high risk” viruses in the medical literature.

Exogenous retroviruses are known to cause various malignant and non-malignant diseases in animals over a wide range of species. These viruses infect most known animals and rodents. Examples include Deltaretroidvirus (HTLV-1, -2, -3, and -4, STLV-1, -2, and -3), Gammaretrovirus (MLV, PERV), Alpharetrovirus (Avian leucosis virus and Avian endogenous virus), and HIV 1 and 2.

Other viral families which are potential adventitious contaminants for which embodiments of the present invention are directed include: Bunyaviridae (LCMV, hantavirus), Herpesviridae (Human herpesviruses 1 through 8, Bovine herpesvirus, Canine herpesvirus and Simian cytomegalovirus), Hepadnaviridae (Hepatitis B virus), Hepeviridae (Hepatitis E virus), Deltavirus (Hepatitis delta virus), Adenoviridae (Human adenoviruses A-F and murine adenovirus), Coronaviridae, Flaviviridae (Bovine viral diarrhea virus, TBE, Yellow fever virus, Dengue viruses 1-4, WNV and hepatitis C virus), Orthomyxoviridae (influenza), Paramyxoviridae (parainfluenza, mumps, measles, RSV, Pneumonia virus of mice, Sendai virus, and Simian parainfluenza virus 5), Togaviridae (Western equine encephalomyelitis virus, rubella), Picornaviridae (Poliovirus types 1-13, coxsackie B, echovirus, rhinovirus, Human hepatitis A, Human coxsackievirus, Human cardiovirus, Human rhinovirus and Bovine rhinovirus), Reoviridae (Mouse rotavirus, reovirus type 3 and Colorado tick fever virus), and Rhabdoviridae (vesicular stomatitis virus).

For example, mouse and hamster cell banks used to make immunogenic agents can be infected with viruses known to be pathogenic to human. Mouse cell banks can carry lymphocytic choriomeningitis virus (LCM), sendai virus, hantaan virus, and/or lactic dehydrogenase virus; hampster cell banks can carry LCM, sendai virus, and/or reovirus type 3. Indeed, commercially available monoclonal antibodies produced from transgenic mouse-derived cells are tested for virus including LCM, Ectromelia (MEV), mouse encephalomyelitis virus (GDVII), Hantaan, MVM, mouse adenovirus (MAV), mouse hepatitis (MHV), pneumonia virus of mice (PVM), Polyoma, Reovirus type 3 (REO-3), Sendai (SeV), virus of epizootic diarrhea of infant mice (EDIM), mouse cytomegalovirus (MCMV), papovavirus K, and LDVH viruses; Thymic Agent virus; bovine virus diarrhea (BVD), infectious bovine rhinotracheitis (IBR), respiratory parainfluenza-3 (PI-3), papillomavirus (BPV) and adenovirus-3 (BAV-3) viruses; and caprine (goat) adenovirus (CAV), herpesvirus (CHV), and arthritis encephalitis virus (CAEV) viruses. See Geigert, CHALLENGE OF CMC REGULATORY COMPLIANCE FOR BIOPHARMACEUTICALS, 109-11 (Springer, New York, N.Y., 2004); BLA reference No. 98-9912, Centocor, Infliximab Detailed Product Review (1997); BioProcessing J. (Fall, 2009).

In some embodiments, the production of an immunogenic agent in a host cell is enhanced by introducing into the cell an additional RNA effector molecule that affects cell growth, cell division, cell viability, apoptosis, the immune response of the cells, nutrient handling, and/or other properties related to cell growth and/or division within the cell. In further embodiments, production is enhanced by introducing into the cell a RNA effector molecule that transiently inhibits expression of immunogenic agents during the growth phase.

### IV. TRANSCRIPTOME

Embodiments of the present invention also provide for a set of transcripts that are expressed in CHO cells, also called “the CHO cell transcriptome”, and further provides siRNA molecules designed to target any one of the transcripts of the CHO cell transcriptome. Uses of the transcriptome in a form of an organized CHO cell transcript sequence database for selecting and designing CHO cell modulating effector RNAs are also provided in the form or methods and systems. Other embodiments further provide a selection of siRNAs targeted against each of the transcripts in the CHO transcriptome, and uses thereof for engineering or modifying CHO cells, for example, for improved production of biomolecules. Accordingly, particular embodiments provide modified CHO cells.

A set of transcripts that were discovered in CHO cells pooled under different conditions, including early-, mid- and late-log phase cells, that were grown in standard conditions under chemically defined media at 37° C. The transcripts are set forth in the tables herein, and in the corresponding sequences (SEQ ID files).

The discovery of the CHO transcriptome is useful for specifically modifying one or more cellular processes in the CHO cell, for example, for the production of biomolecules in such cells. For example, based on the known expressed transcripts, one can modulate apoptosis regulating genes, cell cycle genes, DNA amplification (DHFR) regulating genes, virus gene production regulating genes, e.g., in the case of viral promoters that are used to drive biomolecule production in the cells, glycosylation-associated genes, carbon metabolism regulating genes, prooxidant enzyme encoding genes. By modulating the known expressed genes or transcripts one can further modulate protein folding, methionine oxidation, protein pyroglutamation, disulfide bond formation, protein secretion, cell viability, specific productivity of cell, nutrient requirements, internal cell pH.

Methods for modulating production of an immunogenic agent in a host cell, particularly in a CHO cell, are provided, the methods comprising the steps of contacting the cell with a RNA effector molecule, a portion of which is complementary to at least a portion of a target gene, maintaining the cell in a bioreactor for a time sufficient to modulate expression of the target gene, wherein the modulation enhances production of the immunogenic agent and recovering the immunogenic agent from the cell.

The present disclosure includes the nucleic acid sequences of the transcripts of the CHO transcriptome, the proteins the transcripts are translated into, and some of the pathways in which the transcribed proteins play a role. The description also sets forth a compilation of siRNA molecules as RNA effector molecules designed to target the sequences of the transcriptome. Systems, including computer assisted systems, and methods, including computer assisted methods, for selecting appropriate RNA effector molecules to modulate gene expression in a cell, particularly in a CHO cell, based on the known transcriptome transcript sequences are also described.

CHO Cell Transcriptome:

We have discovered a defined set of transcripts expressed in a CHO cell. The defined set of transcripts in referred to herein as a “transcriptome”. The transcript name, at least one pathway in which the transcript plays a role, an associated SEQ ID NO(s), and corresponding exemplary siRNA molecule SEQ ID NOs are set forth in any of the tables described herein including, for example, Tables 1-16, 21, 23, 24, 27-30, 52-61, 65 or 66. The sequences of the transcripts in the CHO cell transcriptome are set forth in the associated SEQ ID NOs:1-9771 and SEQ ID NOs:3157149-3158420.

Thus, in one embodiment, the invention provides a Chinese hamster ovary (CHO) cell transcriptome comprising a selection or a compilation of transcripts having SEQ ID NOs:1-9771. In some embodiments, the CHO transcriptome consists essentially of a selection or a compilation of transcripts having SEQ ID NOs:1-9771. In some embodiments, the CHO cell transcriptome consists of a selection or a compilation of transcripts having SEQ ID NOs:1-9771.

In some embodiments, the invention provides at least one siRNA directed to any one of the CHO cell transcriptome transcript set forth in any of the tables presented herein, see e.g., Tables 1-16, 21-25, 27-30, 52-61, 65 or 66. In some embodiments, the siRNA is selected from the group of siRNAs set forth in Tables 1-16, 21-31, 33, 35, 37, 39, 41, 43, 45, 47, 49, 50, 51-61, 63-65 or 66. In some embodiments, not all transcript SEQ ID NOs are present in the tables described herein. In some embodiments, the RNA effector molecule comprises an antisense strand comprising at least 16 contiguous nucleotides (e.g., at least 17, at least 18, at least 19 nucleotides) of the nucleotide sequence selected from the group consisting of SEQ ID NOs:9772-3152399 and SEQ ID NOs:3161121-3176783. Additional targets that can be modulated for improved quality/quantity of expression are set forth herein. Provided herein are CHO transcripts, i.e. SEQ ID NO's 1-9771 and SEQ ID NOs:3157149-3158420. These transcripts can be assigned to an encoded protein name and categorized into functional groups. One can readily determine functional groups to classify a transcript to by homology to sequences known to have a particular function. In one embodiment one uses a known functional domain and looks for homology of at least 40%, 50%, 60%, 70%, 75%, 80%, 85%, 90%, 95%. See for example Tables 10-16, which correlate the SEQ ID NO transcript with a description of encoded protein and function, e.g., cell cycle/cell division transcripts of Table 13. Exemplary categories that transcripts can be grouped are described throughout the application and include, e.g., transcripts (i.e., target genes) that encode for proteins involved in apoptosis, cell cycle genes, DNA amplification (DHFR), glycosylation, carbon metabolism, prooxidant enzymes, protein folding, methionine oxidation, protein pyroglutamation, disulfide bond formation, protein secretion, immune response, cell nutrient requirements, and shutting down RNA Interference. For the transcripts disclosed herein whose function is not specifically recited herein, one of skill in the art can easily compare (using known algorithms and programs) the transcript sequences of SEQ ID NOs:1-9771 and SEQ ID NOs:3157149-3158420 to sequence information of transcripts found in any of various organisms and assign function and/or protein encoded name as described above. For example, one of skill in the art can use the sequence information described herein to predict protein function using any prediction methods, algorithms, and/or resources and applications found on the world wide web, as reviewed in any of Freitas et al., 7 IEEE/ACM Transactions on Computational Biology and Bioinformatics (TCBB) 172-82 (2010); Rentzscha & Orengoa, 27 Trends in Biotech. 210-19 (2009); Lowenstein et al., 10 Genome Biol. 207 (2009) or Friedberg, 7 Briefings in Bioinformatics 225-42 (2006). Alternatively, the transcript sequences can be compared to a partial or entire genome of an organism (genome information), including protein coding and non-coding regions.

One can silence target transcripts using siRNA, such as set forth in SEQ ID NOs:9772-3152399 and SEQ ID NOs:3161121-3176783. The particular siRNA can readily be matched to its corresponding target by looking for a transcript containing a complimentary sequence that is at 90% complementary. Well known algorithms can be used to determine appropriate RNA effector molecules for targeting the transcripts identified herein. For example, one of skill in the art can use the sequence information described herein to determine appropriate RNA sequences for targeting the transcripts described herein, and for preventing/promoting an immune response to those RNA sequences, using any prediction methods, algorithms, and/or resources and applications found on the world wide web, as reviewed in, or as described in, Pappas et al., 12 Exp. Op. Therapeutic Targets 115-27 (2008); Kurreck et al., 2009, 48 Angewandte Chemie 1378-98 (2009); Gredell et al., 16 Engin. Cell Funct. by RNA Interference in Cell Engin. 175-94 (2009); PCT/US2005/044662 (Jun. 15, 2006); PCT/US2009/039937 (Oct. 15, 2009); or PCT/US2009/051648 (Jan. 28, 2010).

Thus, the system described herein (i.e., to select for a sequence of at least one RNA effector molecule that is suitable for modulating protein expression in a cell) can be used to identify both the CHO transcript sequence and the RNA effector molecules (e.g., siRNAs) that can be used to modulate any particular function in the host cell. A CHO transcript is assigned function and/or encoded protein name when the transcript sequence has at least 50%, at least 60%, at least 70%, at least 80%, or at least 90% sequence identity to a transcript of an organism whose function and protein name is known

### Systems and Methods for Selecting RNA Effector Molecules:

Based on the known CHO transcriptome, we have developed methods and systems for selecting RNA effector molecules to affect the cells through manipulating cellular processes, for example, to improve production of biomolecules in the cells.

Accordingly, the present embodiments provide databases and system comprising and using the CHO transcriptome sequences and optionally also an organized compilation of the CHO transcriptome outlining at least one functional aspect of each of the transcript, such as the transcripts role in a particular cellular process or pathway, and the corresponding siRNAs to allow design and selection of targets and effector RNA molecules for optimization of biological processes, particularly in the CHO cells.

Functional aspects of transcripts relate to their role in, for example apoptosis, cell cycle, DNA amplification (DHFR), virus gene production, e.g., in the case of viral promoters that are used to drive biomolecule production in the cells, glycosylation, carbon metabolism, prooxidant enzymes, protein folding, methionine oxidation, protein pyroglutamation, disulfide bond formation, protein secretion, cell viability, specific productivity of cell, nutrient requirements, internal cell pH. Other cellular processes are known to a skilled artisan, and can be found, for example, at the Gene Ontology database available through the world wide web.

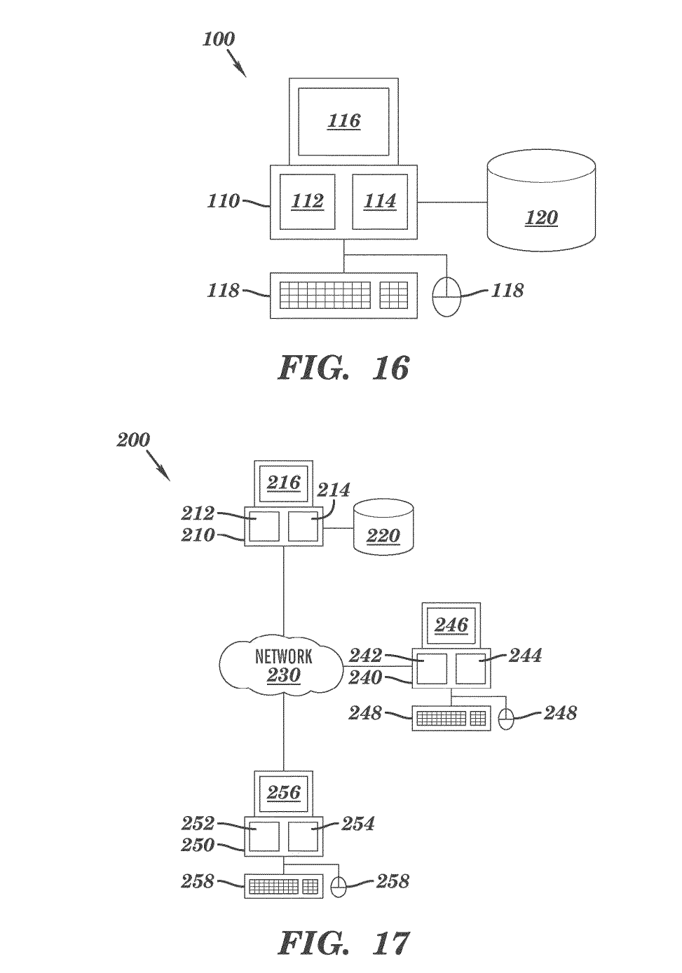

Accordingly as shown in FIG. 16, the invention provides a system 100 for selecting a sequence of at least one RNA effector molecule suitable for modulating protein expression in a cell, the system comprising: a computing device 110, having a processor 112 and associated memory 114, and a database 120 comprising at least one cell transcriptome information, the information comprising, a sequence for each transcript of the transcriptome, and optionally, a name of the transcript, and a pathway the transcript plays a role; and at least one RNA effector molecule information, the information comprising at least the sequence of the RNA effector molecule and optionally target specificity of the RNA effector molecule, wherein each RNA effector molecule is designed to match at least one or more sequences in the at least one cell transcriptome; a computer program, stored in memory 114, executed by the computing device 110 and configured to receive from a user via a user input device 118, parameters comprising a cell type selection, a target organism selection, a cellular pathway selection, a cross-reactivity selection, a target gene name and/or sequence selection, and optionally a method of delivery selection comprising either in vivo or in vitro delivery options; and further optionally user address information; a first module configured to check the parameters against the sequences in the database for a matching combination of the parameters and transcriptome transcript sequences; and a second module to display a selected sequence of at least one RNA effector molecule suitable for modulating protein expression in the cell.

The computing device 110 and associated programs stored in memory 114 can be adapted and configured to provide a user interface, such as a graphical user interface which allows the user to input search target parameters, for example, using one or more drop down menus or structured or free form text input, and selects the appropriate parameters for finding an appropriate target in the desired cell. For example, if a user wishes to find a target for modulating carbon metabolism in a CHO cell, the user identifies the target cell as “CHO”, and pathway as “carbon metabolism”, and the server performs a search through the database that would identify, e.g., transcripts for Gluts, PTEN and LDH genes and matches them with the appropriate siRNA molecules from the siRNA database part. This output information can be presented to the user on a computer display 116 or other output device, such as a printer.

The system can be a stand-alone system or an internet-based system, wherein the computations and selection of effector RNA molecules is performed in same or different locations. As shown in FIG. 16, the transcriptome information can be stored in database 120 and accessed by computing device 110. As used herein, the term database includes any organization of data regardless of whether it is structured or unstructured that allows retrieval of the information requested. The database can be a flat file or set of flat files stored in memory, one or more tables stored in memory, a set of discrete data elements stored in memory. The database can also include any well known database program that allows a user to directly or indirectly (through another program) access the data. Examples of these include MICROSOFT® ACCESS®, and ORACLE® database and MYSQL® open source database.

In an alternative embodiment of the invention shown in FIG. 17, the system 200 can be a network based system. The system 200 can include a server system 210 and one or more client systems 240 and 250 connected to a network 230, such as a private user network or Ethernet, or the Internet. The server system 210 and client systems 240 and 250 can be computing devices as described herein. Server system 210 can include one or more processors 212 and associated memory 214 and one or more computer programs or software adapted and configured to control the operations and functions of the server system 210. The Server system 210 can include one or more network interfaces for connecting via wire or wirelessly to the network 230. Examples of server systems include computer servers based on INTEL® and AMD microprocessor architectures available from Hewlett-Packard Development Co., LP; DELL; and APPLE® Inc.

Client systems 240 and 250 can include one or more processors 242 and 252 and associated memory 244 and 254 and one or more computer programs or software adapted and configured to control the operations and functions of the client systems 240 and 250. The client systems 240 and 250 can include one or more network interfaces for connecting via wire or wirelessly to the network 230. Examples of client systems include desktop and portable computers based on INTEL® and AMD microprocessor architectures available from Hewlett-Packard Development Co., LP; DELL; and Apple Inc., and smaller network enabled, handheld devices such as a personal digital assistant (PDA) (e.g., DROID®, HTC Corp.) smartphone (e.g., BLACKBERRY® smartphone, Research In Motion, Ltd.), iPod®, iPad™ and iPhone® devices (APPLE® Inc.).

In accordance with one embodiment, the server system 210 is a web server, for example based in Internet Information Services (11S) for Windows® or .NET FRAMEWORK products (MICROSOFT® Corp.), or Apache open-source HTTP server (Apache Software Foundation), and uses a web-based application accessed by a remote client system via the Internet to search the database of transcriptome information to identify RNA effector molecules that can be suitable for modulating protein expression in a cell. The system can include or be connected to a fulfillment system that allows a user to select and purchase desired quantities of the identified RNA effector molecules to be delivered to the user.

One can also provide a system by selling a software to be run by a computer, wherein the databases and algorithms matching the parameters with sequence information and other information are provided to the user. The user can then either synthesize the effector RNA molecules or separately order them from a third party provider.

In some embodiments, the system further comprises a storage module for storing the at least one RNA effector molecule in a container, wherein if there are two or more RNA effector molecules, each RNA effector molecule is stored in a separate container, and a robotic handling module, which upon selection of the matching combination, selects a matching container, and optionally adds to the container additives based on a user selection for in vivo or in vitro delivery, and optionally further packages the container comprising the matching RNA effector molecule to be sent to the user address. Exemplary additives that can be added to the siRNA or a mixture of siRNAs are set forth herein.

The storage module can be a refrigerated module linked to the system components.

The system can also be linked to a nucleic acid or other biomolecule synthesizer.

The robotic handling module can be any system that can retrieve, and optionally mix components from the storage module, and or the biomolecule synthesizer, and optionally package the container(s). The robotic handling module can comprise one or more parts functioning based upon the commands from the system. The robotic handling module can be in the same or different location as where the computations are performed.

In some embodiments, the system further comprises genome information of the cell, wherein by a user selection, the RNA effector molecules can be matched to target genomic sequences, comprising promoters, enhancers, introns and exons present in the genome.

In some embodiments of the invention, the system can include hardware components or systems of hardware components and software components that carry out specific tasks (such as managing input and output of information, processing information, etc.) of the system and can be carried out by the execution of software applications on and across the one or more computing devices that make up the system. The present inventions can include any convenient type of computing device, e.g., such as a server, main-frame computer, a work station, etc. Where more than one computing device is present, each device can be connected via any convenient type of communications interconnect, herein referred to as a network, using well know interconnection technologies including, for example, Ethernet (wired or wireless—“WiFi”), BLUETOOTH® technology, ZIGBEE® wireless technology, AT&T™ 3 G network, or SPRINT™ 3 G or 3 G/4 G networks. Where more than one computing device is used, the devices can be co-located or they can be physically separated. Various operating systems can be employed on any of the computing devices, where representative operating systems include MICROSOFT® WINDOWS® operating system, MACOS™ operating system software (APPLE® Inc.), SOLARIS® operating system (Oracle Corp.), Linux (Linux Online, Inc.), UNIX® server systems and OS/400 software (IBM Corp.), ANDROID™ (Sprint), Chrome OS (Google Inc.), and others. The functional elements of system can also be implemented in accordance with a variety of software facilitators, platforms, or other convenient method.

Items of data can be “linked” to one another in a memory when the same data input (for example, filename or directory name or search term) retrieves the linked items (in a same file or not) or an input of one or more of the linked items retrieves one or more of the others.

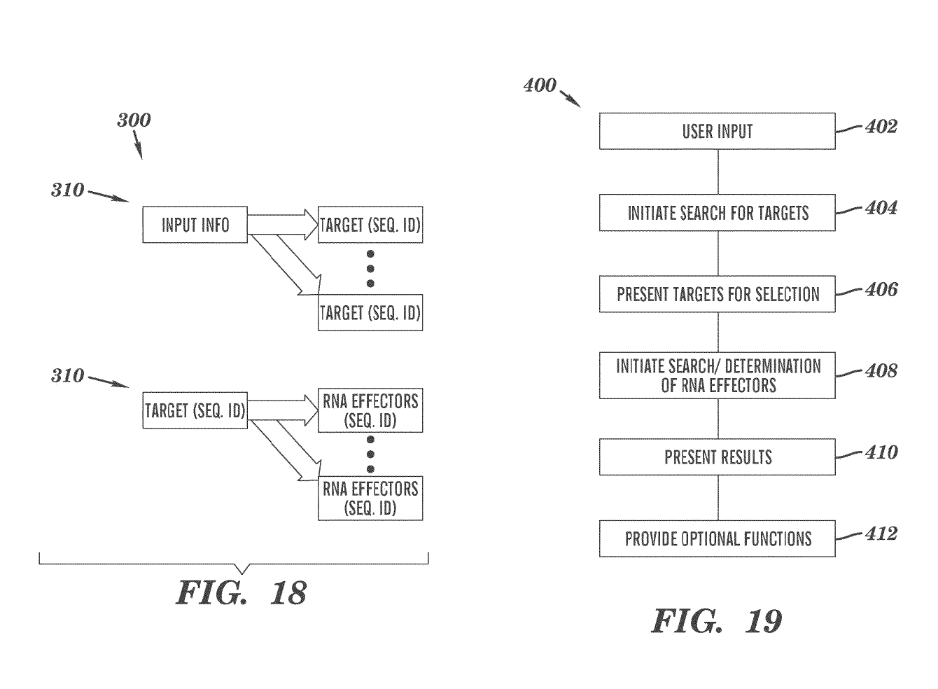

FIG. 18 shows a diagrammatic view of the data structure according to one embodiment of the invention. In this embodiment, input field terms can be linked to Target RNA, such as by their associated sequence ID in the database and in accordance with the invention, executing a software module to search for one or more of the input field terms returns one or more sequence IDs of the Target. In addition, each Target RNA can be linked to one or more RNA effector molecules, such as by their associated sequence ID and in accordance with the invention, the for each Target identified, a software module can be executed to perform a subsequent search for some or all of Targets identified can return one or more sequence IDs for desired RNA effector molecules and return a listing of the RNA effector molecules and their sequence IDs.

Alternatively, for each target identified, a software module can be executed that implements one or more well known algorithms for determining the desired RNA effector molecules and return a listing of the RNA effector molecules and their sequence IDs.

FIG. 19 shows a flow chart of the method for identifying RNA effector molecules according to one embodiment of the invention. The method 400 includes presenting the user with an input screen 402 that allows the user to input the desired parameters for finding the Target in the desired cell. The input can be free form text or one or more drop-down boxes allowing the user to select predefined terms. At step 404, the user selects the appropriate user interface element, for example a “search” button and the system searches the database for Targets associated with the input parameters. At step 406, the user can be presented with a list of Targets, each associated with a check box and the user can select or unselect the check box associated with each target to further refine their search. At step 408, the user selects the appropriate user interface element, for example a “search” button and the system can search the database for RNA effector molecules associated with the input targets and/or use well know algorithms to determine RNA effector molecules associated with the input targets. The system can, for example, search for RNA effector molecules and if, none are found, use the well know algorithms to determine appropriate RNA effector molecules. Subsequently, the determined molecules can be added to the database and appear in subsequent searches. Alternatively, even where RNA effector molecules are found, the system can, in addition, use the well know algorithms to determine additional appropriate RNA effector molecules. At step 410, the user can be provided with optional functions such as ordering the reported RNA effector molecule from information found in the database. For example, online procurement can be provided as described in U.S. Patent Application Pub. No. 2005/0240352.

In one example of the system and the method of using the system, a person, such as a customer, is experiencing problems in protein production using a cell line. The problem can be, e.g., in post translational modification of the protein, such as in glycosylation, e.g., too much fucosylation, and/or another process, such as too much lactic acid buildup or too low yield.

The system of the invention allows the user to input parameters, such as the problem or multiple problems they are experiencing (too low cell growth rate or too much fucosylation) and/or a target gene, or transcript or multiple target genes or transcripts that they wish to modulate, such as FUT8, GMDS, and/or TSTA3, into the user interface.

The system takes the parameters and matches them with sequence data and RNA effector molecule data and delivers suggested RNA effector molecule(s) to the customer. For example, the system can match the problem to a cellular pathway, such as glycosylation, with transcripts known to play a role in glycosylation, and then matches the RNA effector molecules targeting these sequences and delivers, e.g. a list of siRNA sequences with which the customer can experiment.

If the customer wishes to receive one or more of the sequences, the customer can order or instruct the system to synthesize and/or send the appropriate nucleic acids to the customer-defined location. The system can also send instructions to a nucleotide synthesis system to make the sequences. The synthesizer can be in the same or in a remote location from the other system parts. The system can also select ready-made sequences from a storage location and provide packaging information so that the appropriate molecules can be sent to the customer-defined location. If the customer wishes to obtain different mixtures of the RNA effector molecules, such can be defined prior to submitting the final order and then the system will instruct the robotic component to mix the appropriate RNA effector molecules, such as siRNA duplexes, e.g, comprising an antisense and sense strand, in one vial or tube or other container.

We have further discovered a set of siRNA molecules that target at least one of the transcripts in the CHO cell transcriptome. Table 1 also sets forth a set of siRNA molecules that target the transcripts in the CHO cell transcriptome.

Thus, for example, methods are provided herein for enhancing production of a recombinant antibody or a portion or derivative thereof by contacting a cell, such as a CHO cell, with one or more RNA effector molecules that permit modulation of fucosylation of the recombinant antibody or portion or derivative thereof. For example, SEQ ID NOs:3152714-3152753, can be contacted with a cell to modulate expression of the fucosyltransferase (FUT8). In another embodiment, a cell is contacted with one or more RNA effector molecules wherein the contacting modulates expression of a GDP0mannose 4,6-dehydratase (GMDS) (encoded, for example, by SEQ ID NO:5069). A RNA effector molecule targeting GMDS can comprise an antisense strand comprising at least 16 contiguous nucleotides (e.g., at least 17, at least 18, at least 19 nucleotides) of the oligonucleotide having a nucleotide sequence selected from the group consisting of SEQ ID NOs:1688202-1688519.

In another embodiment, a cell is contacted with one or more RNA effector molecules wherein the contacting modulates expression of a gene encoding GDP-4-keto-6-deoxy-D-mannose epimerase-reductase (encoded by TSTA3), (encoded, for example, by SEQ ID NO:5505). A RNA effector molecule targeting TSTA3 can comprise an antisense strand comprising at least 16 contiguous nucleotides (e.g., at least 17, at least 18, at least 19 nucleotides) of an oligonucleotide molecule selected from the group consisting of SEQ ID NOs:1839578-1839937. In still another embodiment, a cell is contacted with a plurality of RNA effector molecules targeting the expression of more than one of FUT8, GMDS, and TSTA3.

Reduced sialic content of antibodies is believed to further increase ADCC. Therefore, in still another embodiment, a cell is contacted with one or more RNA effector molecules wherein the contacting modulates expression of a sialyltransferase. The sialyltransferase activity in a cell can be modulated by contacting the cell with a RNA effector molecule targeting at least one sialyltransferase gene. Table 7 lists some sialyltransferases that can be modulated, as well as the RNA effector molecules targeting sialyltransferases.

The RNA effector molecules targeting the sialyltransferases comprises an antisense strand comprising at least 16 contiguous nucleotides (e.g., at least 17, at least 18, at least 19 nucleotides) of the oligonucleotide having a nucleotide sequence of the SEQ ID NOs presented above (i.e., SEQ ID NOs:681105-681454, NOs:707535-707870, NOs:1131123-1131445, NOs:1155324-1155711, NOs:1391079-1391449, NOs:1435989-1436317).

In still another embodiment, a cell is contacted with at least one RNA effector molecule targeting one of FUT8, GMDS, and TSTA3, and another RNA effector molecule targeting one sialyltransferase. In a particular embodiment, a cell is contacted with RNA effector molecules targeting FUT8 and ST6 (α-N-acetyl-neuraminyl-2,3-β-galactosyl-1,3)-N-acetylgalactosaminide α-2,6-sialyltransferase 6.

Embodiments of the present invention modulated the activity of a transcript or a protein in a molecular pathway known to a skilled artisan or identified elsewhere in this specification. Such molecular pathways an cellular activities include, but are not limited to apoptosis, cell division, glycosylation, growth rate, a cellular productivity, a peak cell density, a sustained cell viability, a rate of ammonia production or consumption, or a rate of lactate production. Tables 10 to 16 identify example targets based on their function or role that they play in a cell:

TABLE 10
Lactate production (Chinese hamster)

| | | | Avg | siRNA |
|---|---|---|---|---|
| SEQ ID NO: | consL | Description | Cov | SEQ ID NOs: |
| 3905 | 1573 | lactate dehydrogenase A | 1,468.00 | 1297283-1297604 |
| 8572 | 481 | lactate dehydrogenase C | 0.619 | 2887819-2888178 |
| 9187 | 343 | lactate dehydrogenase A-like 6B | 0.235 | 3064087-3064357 |
| 9600 | 207 | lactate dehydrogenase B | 0.216 | 3140011-3140113 |

TABLE 11
Proteases and Proteolysis related (Chinese hamster)

| SEQ | | | Avg | siRNA SEQ |
|---|---|---|---|---|
| ID NO: | consL | Description | Cov | ID NOs: |
| 6 | 5005 | carboxypeptidase D | 5.679 | 11367-11661 |
| 23 | 4373 | insulin degrading enzyme | 24.134 | 16605-16843 |
| 151 | 3548 | disintegrin & metallopeptidase domain 10 | 14.497 | 57423-57713 |
| 282 | 3138 | YME1-like 1 (S. cerevisiae) | 5.064 | 96707-96922 |
| 351 | 3031 | SUMO/sentrin specific peptidase 6 | 10.532 | 116231-116447 |
| 360 | 3012 | bone morphogenetic protein 1 | 14.594 | 118879-119164 |
| 367 | 3002 | dipeptidylpeptidase 8 | 4.382 | 120799-121136 |
| 450 | 2894 | tripeptidyl peptidase II | 4.093 | 144491-144745 |
| 462 | 2883 | nardilysin, N-Arg dibasic convertase, | 23.889 | 147663-147880 |
| | | NRD convertase 1 | | |
| 483 | 2861 | calpain 2 | 35.121 | 153383-153617 |
| 544 | 2789 | N-ethylmaleimide sensitive fusion protein | 30.345 | 170769-171035 |
| 557 | 2776 | disintegrin & metallopeptidase domain 9 | 15.711 | 174168-174399 |
| | | (meltrin γ) | | |
| 582 | 2754 | Zn metallopeptidase, STE24 homolog | 5.717 | 181477-181863 |
| | | (S. cerevisiae) | | |
| 677 | 2678 | AE binding protein 1 | 54.178 | 210228-210444 |
| 816 | 2577 | disintegrin and metallopeptidase domain 23 | 0.593 | 252647-252954 |
| 821 | 2575 | a disintegrin and metallopeptidase domain | 11.757 | 254091-254472 |
| | | 15 (metargidin) | | |
| 940 | 2519 | SUMO/sentrin specific peptidase 2 | 3.997 | 292258-292589 |
| 1012 | 2474 | membrane-bound transcription factor | 14.435 | 316272-316622 |
| | | peptidase, site 1 | | |
| 1064 | 2446 | lon peptidase 1, mitochondrial | 39.647 | 333731-334048 |
| 1108 | 2423 | AFG3(ATPase family gene 3)-like 2 (yeast) | 17.55 | 348153-348484 |
| 1137 | 2407 | acylpeptide hydrolase | 16.618 | 358347-358692 |
| 1153 | 2401 | calpain 10 | 2.795 | 363875-364249 |
| 1194 | 2384 | disintegrin-like & metallopeptidase (reprolysin | 8.75 | 377552-377859 |
| | | type) with thrombospondin type 1 motif, 7 | | |
| 1330 | 2323 | complement component 1, r subcomponent | 62.586 | 422509-422751 |
| 1331 | 2323 | pitrilysin metallepetidase 1 | 16.737 | 422752-423147 |
| 1365 | 2304 | X-prolyl aminopeptidase (aminopeptidase P) 1 | 34.448 | 434820-435212 |
| 1367 | 2303 | neurolysin (metallopeptidase M3 family) | 4.852 | 435611-435974 |
| 1423 | 2276 | plasminogen activator, tissue | 2.837 | 454515-454869 |
| 1462 | 2261 | SUMO/sentrin specific peptidase 3 | 7.248 | 467735-468057 |
| 1488 | 2250 | furin (paired basic aa cleaving enzyme) | 14.282 | 476518-476914 |
| 1554 | 2228 | SUMO/sentrin specific peptidase 5 | 1.726 | 498550-498878 |
| 1597 | 2208 | aminopeptidase puromycin sensitive | 4.993 | 513207-513606 |
| 1601 | 2208 | complement component 1, s subcomponent | 7.355 | 514675-514999 |
| 1703 | 2174 | endoplasmic reticulum aminopeptidase 1 | 16.062 | 550016-550337 |
| 1828 | 2136 | matrix metallopeptidase 9 | 16.328 | 593202-593492 |
| 1832 | 2133 | endoplasmic reticulum metallopeptidase 1 | 3.502 | 594506-594744 |
| 1861 | 2124 | spastic paraplegia 7 homolog (human) | 8.718 | 604347-604631 |
| 1980 | 2085 | complement component 1, r subcomponent B | 28.837 | 644971-645023 |
| 1989 | 2082 | thimet oligopeptidase 1 | 27.953 | 647877-648172 |
| 2005 | 2076 | beta-site APP cleaving enzyme 1 | 3.234 | 653217-653567 |
| 2034 | 2066 | intraflagellar transport 52 homolog | 44.311 | 662569-662878 |
| | | (Chlamydomonas) | | |
| 2060 | 2056 | dihydrolipoamide dehydrogenase | 39.837 | 671424-671769 |
| 2086 | 2048 | methionyl aminopeptidase 1 | 16.104 | 680457-680813 |
| 2093 | 2046 | cathepsin A | 183.096 | 682818-683174 |
| 2109 | 2041 | disintegrin-like & metallopeptidase (reprolysin | 0.788 | 687923-688239 |
| | | type) with thrombospondin type 1 motif, 1 | | |
| 2352 | 1970 | ATP/GTP binding protein-like 5 | 1.205 | 770448-770765 |
| 2369 | 1965 | cathepsin D | 167.968 | 776029-776328 |
| 2370 | 1965 | methionine aminopeptidase 2 | 19.432 | 776329-776680 |
| 2440 | 1946 | arginyl aminopeptidase (aminopeptidase B) | 9.264 | 800159-800460 |
| 2473 | 1940 | prolyl endopeptidase-like | 2.435 | 811154-811532 |
| 2521 | 1929 | dipeptidylpeptidase 9 | 4.703 | 827728-828118 |
| 2529 | 1926 | AFG3 (ATPase family gene 3)-like 1 (yeast) | 8.094 | 830536-830879 |
| 2549 | 1920 | leukotriene A4 hydrolase | 13.262 | 837346-837737 |
| 2627 | 1901 | tubulointerstitial nephritis antigen-like 1 | 471.915 | 863337-863698 |
| 2688 | 1887 | prolylcarboxypeptidase (angiotensinase C) | 4.268 | 884238-884577 |
| 2726 | 1875 | CNDP dipeptidase 2 (metallopeptidase | 17.92 | 897182-897473 |
| | | M20 family) | | |
| 2802 | 1857 | legumain | 105.23 | 923229-923566 |
| 2867 | 1840 | cereblon | 1.831 | 945414-945728 |
| 2888 | 1834 | cathepsin F | 27.16 | 952584-952981 |
| 2902 | 1830 | proprotein convertase subtilisin/kexin type 7 | 5.151 | 957525-957819 |
| 2940 | 1818 | OMA1 homolog, zinc metallopeptidase | 10.717 | 970455-970848 |
| | | (S. cerevisiae) | | |
| 2957 | 1814 | disintegrin & metallopeptidase domain 22 | 6.245 | 976428-976826 |
| 2962 | 1812 | bleomycin hydrolase | 21.221 | 978233-978617 |
| 3044 | 1781 | leucine aminopeptidase 3 | 53.967 | 1005879-1006172 |
| 3119 | 1765 | prolyl endopeptidase | 20.21 | 1031521-1031842 |
| 3129 | 1763 | matrix metallopeptidase 3 | 44.776 | 1034832-1035193 |
| 3175 | 1751 | disintegrin & metallopeptidase domain 8 | 3.157 | 1051064-1051435 |
| 3296 | 1720 | suppression of tumorigenicity 14 | 2.378 | 1092011-1092357 |
| | | (colon carcinoma) | | |
| 3347 | 1706 | LON peptidase N-terminal domain & ring | 1.265 | 1109135-1109435 |
| | | finger 3 | | |
| 3515 | 1666 | calpain 7 | 1.488 | 1165709-1166037 |
| 3553 | 1656 | peptidase (mitochondrial processing) | 16.51 | 1178516-1178823 |
| 3565 | 1652 | HtrA serine peptidase 1 | 42.699 | 1182505-1182824 |
| 3660 | 1631 | aspartyl aminopeptidase | 12.181 | 1214496-1214794 |
| 3685 | 1627 | HtrA serine peptidase 2 | 11.095 | 1222907-1223252 |
| 3696 | 1623 | intraflagellar transport 88 homolog | 1.53 | 1226651-1227010 |
| | | (Chlamydomonas) | | |
| 3770 | 1607 | a disintegrin and metallopeptidase | 0.371 | 1251949-1252245 |
| | | domain 12 (meltrin) | | |
| 3795 | 1599 | ubiquinol-cytochrome c reductase core | 109.161 | 1260523-1260890 |
| | | protein 1 | | |
| 3809 | 1594 | matrix metallopeptidase 10 | 43.632 | 1265238-1265630 |
| 3832 | 1589 | matrix metallopeptidase 14 | 5.689 | 1272953-1273286 |
| | | (membrane-inserted) | | |
| 3875 | 1579 | peptidase (mitochondrial processing) β | 37.799 | 1287161-1287545 |
| 3936 | 1565 | predicted gene 5077 | 4.951 | 1307451-1307521 |
| 3940 | 1564 | dipeptidylpeptidase 7 | 40.962 | 1308543-1308899 |
| 3951 | 1562 | phosphatidylinositol glycan anchor | 26.236 | 1312259-1312656 |
| | | biosynthesis, class K | | |
| 4040 | 1540 | cathepsin B | 122.173 | 1342187-1342544 |
| 4112 | 1521 | leucyl/cystinyl aminopeptidase | 0.363 | 1366088-1366414 |
| 4134 | 1516 | mitochondrial intermediate peptidase | 1.762 | 1373601-1373949 |
| 4136 | 1515 | calpain 1 | 1.667 | 1374276-1374636 |
| 4234 | 1494 | WAP, FS, Ig, KU, and NTR- | 1.307 | 1407418-1407713 |
| | | containing protein 1 | | |
| 4250 | 1492 | caspase 9 | 1.769 | 1412589-1412860 |
| 4282 | 1485 | matrix metallopeptidase 12 | 15.393 | 1423446-1423812 |
| 4320 | 1476 | peptidase D | 6.708 | 1436318-1436664 |
| 4345 | 1471 | procollagen C-endopeptidase | 38.334 | 1444649-1444973 |
| | | enhancer protein | | |
| 4515 | 1433 | ceroid lipofuscinosis, neuronal 3, juvenile | 2.904 | 1500552-1500853 |
| | | (Batten, Spielmeyer-Vogt disease) | | |
| 4548 | 1426 | ubiquinol cytochrome c reductase core protein 2 | 74.045 | 1511637-1511998 |
| 4736 | 1385 | cathepsin L | 394.561 | 1574335-1574708 |
| 4999 | 1324 | aminoacylase 1 | 16.465 | 1664426-1664734 |
| 5080 | 1303 | protease, serine, 36 | 0.737 | 1691971-1692344 |
| 5266 | 1267 | tripeptidyl peptidase I | 0.706 | 1755385-1755682 |
| 5334 | 1251 | O-sialoglycoprotein endopeptidase-like 1 | 1.425 | 1778801-1779170 |
| 5395 | 1238 | SUMO/sentrin specific peptidase 8 | 1.488 | 1800688-1801060 |
| 5486 | 1216 | glutaminyl-peptide cyclotransferase-like | 2.05 | 1832626-1832993 |
| 5520 | 1207 | carboxypeptidase X 1 (M14 family) | 0.795 | 1844883-1845160 |
| 5529 | 1205 | glutamyl aminopeptidase | 0.69 | 1847806-1848189 |
| 5550 | 1200 | disintegrin & metallopeptidase domain 17 | 1.374 | 1855220-1855596 |
| 5578 | 1195 | proteasome (prosome, macropain) type 1 | 94.105 | 1864684-1865015 |
| 5608 | 1188 | caspase 12 | 0.856 | 1875252-1875646 |
| 5663 | 1175 | CASP8 and FADD-like apoptosis regulator | 4.448 | 1894743-1895132 |
| 5712 | 1164 | ATP/GTP binding protein 1 | 0.455 | 1912461-1912860 |
| 5746 | 1157 | caspase 3 | 11.813 | 1924836-1925195 |
| 5760 | 1154 | archaelysin family metallopeptidase 2 | 3.826 | 1930073-1930404 |
| 5792 | 1147 | matrix metallopeptidase 13 | 0.724 | 1941794-1942151 |
| 5854 | 1136 | caspase 1 | 2.306 | 1964106-1964500 |
| 5905 | 1123 | RAB23, member RAS oncogene family | 1.099 | 1982920-1983307 |
| 5940 | 1116 | cathepsin H | 23.003 | 1995676-1996039 |
| 5976 | 1108 | SEC11 homolog A (S. cerevisiae) | 44.235 | 2008739-2009125 |
| 6015 | 1099 | proteasome (prosome, macropain) 26S | 63.204 | 2022843-2023145 |
| | | subunit, non-ATPase, 8 | | |
| 6033 | 1095 | protease, serine 27 | 3.375 | 2029351-2029692 |
| 6044 | 1093 | proteasome (prosome, macropain) type 4 | 77.041 | 2033365-2033746 |
| 6101 | 1080 | matrix metallopeptidase 23 | 2.487 | 2053947-2054295 |
| 6154 | 1068 | cathepsin Z | 400.641 | 2073581-2073970 |
| 6247 | 1047 | ceroid-lipofuscinosis, neuronal 6 | 3.41 | 2107037-2107394 |
| 6327 | 1029 | calpain 5 | 2.411 | 2135026-2135381 |
| 6344 | 1025 | C2 calcium-dependent domain containing 3 | 0.136 | 2141185-2141522 |
| 6512 | 985 | proteasome (prosome, macropain) type 5 | 77.333 | 2200953-2201317 |
| 6552 | 976 | endothelin converting enzyme 2 | 2.313 | 2215190-2215580 |
| 6611 | 966 | proteasome (prosome, macropain) type 3 | 3.156 | 2236096-2236486 |
| 6656 | 957 | proteasome (prosome, macropain) type 6 | 42.616 | 2251849-2252237 |
| 6686 | 950 | apoptotic peptidase activating factor 1 | 0.325 | 2262408-2262743 |
| 6745 | 936 | proteasome (prosome, macropain) β type 8 | 32.531 | 2282619-2282981 |
| | | (large multifunctional peptidase 7) | | |
| 6769 | 933 | proteasome (prosome, macropain) β type 10 | 3.428 | 2291135-2291518 |
| 6798 | 926 | caspase 7 | 0.436 | 2301618-2301960 |
| 6818 | 920 | proteasome (prosome, macropain) β type 7 | 44.299 | 2308285-2308647 |
| 6848 | 914 | proteasome (prosome, macropain) β type 4 | 25.753 | 2318721-2319092 |
| 6967 | 888 | proteasome (prosome, macropain) β type 1 | 101.582 | 2357085-2357484 |
| 6999 | 880 | caseinolytic peptidase, ATP-dependent, | 23.993 | 2368027-2368394 |
| | | proteolytic subunit homolog (E. coli) | | |
| 7109 | 858 | matrix metallopeptidase 19 | 0.305 | 2404764-2405144 |
| 7120 | 855 | caspase 6 | 4.965 | 2408466-2408843 |
| 7300 | 811 | proteasome (prosome, macropain) type 7 | 52.239 | 2467566-2467883 |
| 7433 | 780 | proteasome (prosome, macropain) β type 5 | 25.65 | 2511900-2512253 |
| 7532 | 756 | cathepsin O | 0.321 | 2544359-2544680 |
| 7563 | 747 | proteasome (prosome, macropain) type 2 | 6.117 | 2554532-2554886 |
| 7620 | 734 | proteasome (prosome, macropain) β type 3 | 8.915 | 2572635-2572964 |
| 7721 | 709 | aurora kinase A interacting protein 1 | 9.974 | 2606127-2606501 |
| 7782 | 693 | ATP/GTP binding protein-like 3 | 0.407 | 2627002-2627350 |
| 7940 | 648 | matrix metallopeptidase 17 | 0.224 | 2680510-2680844 |
| 7948 | 646 | pyroglutamyl-peptidase I | 0.831 | 2683195-2683515 |
| 7979 | 638 | protease, serine, 8 (prostasin) | 0.479 | 2693206-2693562 |
| 8026 | 624 | CASP2 and RIPK1 domain containing | 1.176 | 2709036-2709355 |
| | | adaptor with death domain | | |
| 8056 | 612 | caspase 2 | 1.166 | 2718675-2719039 |
| 8255 | 558 | matrix metallopeptidase 24 | 6.978 | 2781318-2781710 |
| 8290 | 549 | proteasome (prosome, macropain) β type 2 | 3.953 | 2793443-2793832 |
| 8352 | 535 | IMP1 inner mitochondrial membrane | 6.039 | 2814696-2815033 |
| | | peptidase-like (S. cerevisiae) | | |
| 8440 | 510 | disintegrin-like & metallopeptidase (reprolysin | 0.139 | 2845165-2845528 |
| | | type) with thrombospondin type 1 motif, 4 | | |
| 8466 | 504 | proteasome (prosome, macropain) β type 6 | 1.2 | 2853165-2853489 |
| 8547 | 487 | small optic lobes homolog (Drosophila) | 0.173 | 2879932-2880319 |
| 8577 | 481 | calpain 11 | 0.187 | 2889008-2889328 |
| 8597 | 477 | mannan-binding lectin serine peptidase 2 | 0.156 | 2896069-2896411 |
| 8653 | 465 | membrane-bound transcription factor | 0.105 | 2915060-2915410 |
| | | peptidase, site 2 | | |
| 8917 | 414 | caspase 8 | 0.2 | 2995593-2995870 |
| 8935 | 409 | carboxypeptidase N, polypeptide 1 | 0.233 | 3000705-3001032 |
| 8980 | 398 | disintegrin & metallopeptidase domain 19 | 0.707 | 3012906-3013172 |
| | | (meltrin β) | | |
| 9067 | 373 | proteasome (prosome, macropain) subunit, β | 0.464 | 3035689-3035987 |
| | | type 9 (large multifunctional peptidase 2) | | |
| 9119 | 360 | SUMO1/sentrin specific peptidase 1 | 0.104 | 3048694-3048900 |
| 9253 | 329 | phosphate regulating gene with homologies to | 0.053 | 3078631-3078850 |
| | | endopeptidases on the X chromosome | | |
| | | (hypophosphatemia, vitamin D resistant rickets) | | |
| 9290 | 319 | carboxypeptidase B2 (plasma) | 0.216 | 3086591-3086854 |
| 9365 | 296 | cathepsin W | 0.241 | 3102885-3103082 |
| 9403 | 282 | RIKEN cDNA 4930486L24 gene | 0.203 | 3109975-3110173 |
| 9412 | 278 | cDNA sequence BC039632 | 0.114 | 3111726-3111929 |
| 9418 | 275 | IMP2 inner mitochondrial membrane | 0.242 | 3112815-3113006 |
| | | peptidase-like (S. cerevisiae) | | |
| 9498 | 244 | calpain 12 | 0.103 | 3126461-3126617 |
| 9517 | 238 | mucosa associated lymphoid tissue | 0.359 | 3129264-3129311 |
| | | lymphoma translocation gene 1 | | |
| 9529 | 234 | disintegrin & metallopeptidase domain 1a | 0.077 | 3130955-3131114 |
| 9574 | 215 | SUMO1/sentrin specific peptidase 7 | 0.045 | 3137116-3137276 |
| 9627 | 195 | cathepsin 8 | 0.092 | 3142354-3142386 |
| 9644 | 188 | proteasome (prosome, macropain) β type, 11 | 0.052 | 3143952-3143972 |
| 9647 | 187 | disintegrin & metallopeptidase domain 28 | 0.137 | 3144200-3144221 |
| 9669 | 175 | methionine aminopeptidase-like 1 | 0.139 | 3146223-3146337 |
| 3157186 | 770 | SEC11 homolog C (S. cerevisiae) | 22.702 | 3178484-3178583 |
| 3157231 | 468 | macrophage stimulating 1 (hepatocyte | 0.205 | 3240817-3240916 |
| | | growth factor-like) | | |
| 3157254 | 428 | transferrin receptor 2 | 0.148 | 3252917-3253016 |
| 3157343 | 370 | predicted gene 1019 | 0.391 | 3193971-3194070 |
| 3157354 | 430 | cathepsin K | 0.29 | 3278249-3278348 |
| 3157355 | 419 | calpain 8 | 0.461 | 3258905-3259004 |
| 3157374 | 287 | carnosine dipeptidase 1 (metallopeptidase | 0.102 | 3245017-3245116 |
| | | M20 family) | | |
| 3157412 | 788 | dipeptidylpeptidase 10 | 0.189 | 3248617-3248716 |
| 3157448 | 1697 | folate hydrolase | 1.451 | 3185871-3185970 |
| 3157520 | 492 | complement component 1, r subcomponent-like | 0.264 | 3224791-3224890 |
| 3157628 | 194 | disintegrin & metallopeptidase domain 33 | 0.061 | 3206058-3206157 |
| 3157660 | 369 | echinoderm microtubule associated protein | 0.16 | 3266705-3266804 |
| | | like 2 | | |
| 3157845 | 837 | mast cell protease 8 | 4.869 | 3206558-3206657 |
| 3157898 | 422 | disintegrin-like & metallopeptidase | 0.115 | 3193471-3193570 |
| | | (reprolysin type) with thrombospondin | | |
| | | type 1 motif, 15 | | |
| 3157899 | 306 | napsin A aspartic peptidase | 0.207 | 3240917-3241016 |
| 3157906 | 387 | cathepsin S | 0.283 | 3272096-3272195 |
| 3157949 | 477 | protein C | 0.42 | 3271796-3271895 |
| 3158015 | 396 | mast cell protease 4 | 0.405 | 3210058-3210157 |
| 3158034 | 923 | HtrA serine peptidase 3 | 0.583 | 3258505-3258604 |
| 3158065 | 1746 | WD repeat domain 7 | 1.717 | 3273496-3273595 |
| 3158090 | 371 | secernin 2 | 0.243 | 3163021-3163120 |
| 3158135 | 418 | mannan-binding lectin serine peptidase 1 | 0.152 | 3282249-3282348 |
| 3158156 | 463 | NA | 0.451 | 3181384-3181483 |
| 3158177 | 415 | NA | 0.13 | 3231817-3231916 |
| 3158199 | 521 | hepatocyte growth factor | 0.226 | 3253417-3253516 |
| 3158201 | 416 | matrix metallopeptidase 21 | 0.224 | 3195471-3195570 |
| 3158231 | 385 | matrix metallopeptidase 16 | 0.085 | 3174184-3174283 |
| 3158246 | 338 | coagulation factor VII | 0.181 | 3207558-3207657 |
| 3158294 | 648 | matrix metallopeptidase 2 | 0.413 | 3214291-3214390 |
| 3158365 | 431 | complement component factor i | 0.209 | 3178584-3178683 |
| 3158378 | 492 | alanyl (membrane) aminopeptidase | 0.144 | 3228717-3228816 |

TABLE 12
Extracellular Space; External Region (Chinese hamster)

| SEQ | | | Avg | siRNA SEQ |
|---|---|---|---|---|
| ID NO: | consL | Description | Cov | ID NOs: |
| 7 | 4892 | collagen, type IV, 2 | 29.59 | 11662-12024 |
| 10 | 4667 | collagen, type V, 1 | 22.034 | 12499-12766 |
| 40 | 4217 | collagen, type IV, 1 | 71.884 | 22106-22419 |
| 53 | 4076 | laminin B1 subunit 1 | 72.723 | 26303-26608 |
| 68 | 3989 | laminin, γ 1 | 8.547 | 31249-31602 |
| 72 | 3984 | nidogen 1 | 31.556 | 32592-32943 |
| 98 | 3777 | neural cell adhesion molecule 1 | 1.452 | 41193-41507 |
| 99 | 3776 | inter-(globulin) inhibitor H5 | 3.94 | 41508-41833 |
| 106 | 3741 | latent TGF β binding protein 1 | 15.581 | 43659-44014 |
| 122 | 3653 | laminin, 5 | 10.318 | 48814-49139 |
| 150 | 3549 | UDP-N-acetyl--D-galactosamine:polypeptide | 11.757 | 57147-57422 |
| | | N-acetylgalactosaminyl transferase 1 | | |
| 168 | 3455 | activated leukocyte cell adhesion molecule | 11.813 | 62634-62891 |
| 178 | 3411 | UDP-N-acetyl--D-galactosamine:polypeptide | 22.835 | 65737-65999 |
| | | N-acetylgalactosaminyl transferase 2 | | |
| 188 | 3385 | fibronectin 1 | 39.064 | 68761-69090 |
| 228 | 3262 | collagen, type XII, 1 | 0.842 | 80671-81033 |
| 266 | 3179 | vascular endothelial growth factor A | 18.713 | 92246-92594 |
| 296 | 3122 | calumenin | 31.456 | 101047-101312 |
| 331 | 3068 | collagen, type XVI, 1 | 16.307 | 110363-110636 |
| 373 | 2991 | CD44 antigen | 11.502 | 122703-122982 |
| 374 | 2990 | ring finger and SPRY domain containing 1 | 5.312 | 122983-123259 |
| 392 | 2965 | lysyl oxidase-like 4 | 3.371 | 128072-128461 |
| 428 | 2922 | coiled-coil domain containing 80 | 7.726 | 138093-138362 |
| 435 | 2913 | low density lipoprotein receptor-related protein | 3.732 | 140196-140578 |
| | | 8, apolipoprotein e receptor | | |
| 546 | 2787 | DnaJ (Hsp40) homolog, subfamily C, member 10 | 22.023 | 171304-171555 |
| 557 | 2776 | disintegrin & metallopeptidase domain 9 | 15.711 | 174168-174399 |
| | | (meltrin γ) | | |
| 602 | 2739 | lysyl oxidase-like 3 | 1.964 | 187446-187711 |
| 655 | 2695 | perlecan (heparan sulfate proteoglycan 2) | 13.274 | 203335-203554 |
| 677 | 2678 | AE binding protein 1 | 54.178 | 210228-210444 |
| 679 | 2677 | collagen, type VI, 1 | 34.848 | 210698-211081 |
| 703 | 2663 | RIKEN cDNA 2610507B11 gene | 20.912 | 217294-217526 |
| 704 | 2662 | serine (or cysteine) peptidase inhibitor, | 33.405 | 217527-217924 |
| | | clade E, member 1 | | |
| 726 | 2641 | collagen, type VI, 2 | 42.145 | 224615-225009 |
| 798 | 2590 | collagen & calcium binding EGF domains 1 | 2.683 | 246931-247299 |
| 816 | 2577 | disintegrin & metallopeptidase domain 23 | 0.593 | 252647-252954 |
| 885 | 2543 | platelet-derived growth factor, C polypeptide | 3.586 | 273882-274243 |
| 941 | 2519 | heat shock protein 5 | 729.81 | 292590-292837 |
| 956 | 2506 | integrin 5 (fibronectin receptor) | 13.308 | 297403-297671 |
| 968 | 2500 | acid phosphatase-like 2 | 10.599 | 301329-301569 |
| 971 | 2499 | WNT1 inducible signaling pathway protein 1 | 3.327 | 302229-302482 |
| 986 | 2492 | thrombospondin 1 | 2.743 | 307445-307775 |
| 1014 | 2473 | tissue inhibitor of metalloproteinase 2 | 22.337 | 317000-317395 |
| 1034 | 2463 | sema domain, immunoglobulin domain (Ig), | 15.39 | 323916-324170 |
| | | short basic domain, secreted, (semaphorin) 3B | | |
| 1059 | 2448 | glypican 6 | 2.853 | 332251-332483 |
| 1079 | 2437 | thrombospondin 3 | 16.07 | 338433-338822 |
| 1149 | 2404 | MAM domain containing 2 | 23.86 | 362422-362815 |
| 1194 | 2384 | disintegrin-like & metallopeptidase (reprolysin | 8.75 | 377552-377859 |
| | | type) with thrombospondin type 1 motif, 7 | | |
| 1216 | 2374 | integrin V | 0.85 | 384630-384864 |
| 1274 | 2346 | quiescin Q6 sulfhydryl oxidase 1 | 17.49 | 403798-404029 |
| 1307 | 2332 | laminin, β 2 | 3.856 | 414909-415222 |
| 1382 | 2293 | CD276 antigen | 2.822 | 440554-440858 |
| 1408 | 2285 | TBC1 domain family, member 15 | 5.501 | 449214-449575 |
| 1423 | 2276 | plasminogen activator, tissue | 2.837 | 454515-454869 |
| 1424 | 2276 | connective tissue growth factor | 6.301 | 454870-455117 |
| 1529 | 2238 | interleukin 6 signal transducer | 1.155 | 490131-490451 |
| 1583 | 2214 | cleft lip & palate associated transmembrane | 6.218 | 508317-508686 |
| | | protein 1 | | |
| 1587 | 2213 | collagen, type XXVII, 1 | 0.476 | 509761-510121 |
| 1662 | 2187 | ecto-NOX disulfide-thiol exchanger 2 | 1.262 | 536177-536522 |
| 1681 | 2181 | brain derived neurotrophic factor | 1.421 | 542519-542783 |
| 1694 | 2176 | toll-like receptor 2 | 12.95 | 547130-547467 |
| 1700 | 2175 | transforming growth factor, β receptor II | 17.68 | 549106-549395 |
| 1713 | 2171 | lysyl oxidase-like 1 | 27.43 | 553603-553837 |
| 1723 | 2167 | prosaposin | 159.42 | 556999-557313 |
| 1728 | 2165 | leprecan 1 | 29.15 | 558793-559105 |
| 1785 | 2150 | tuftelin 1 | 6.024 | 578466-578777 |
| 1792 | 2147 | family with sequence similarity 108, member B | 12.36 | 580929-581285 |
| 1801 | 2142 | biglycan | 335.92 | 584020-584336 |
| 1828 | 2136 | matrix metallopeptidase 9 | 16.328 | 593202-593492 |
| 1831 | 2134 | dystroglycan 1 | 3.205 | 594147-594505 |
| 1841 | 2131 | glypican 1 | 9.404 | 597502-597879 |
| 1843 | 2130 | lysosomal-associated membrane protein 1 | 239.94 | 598208-598530 |
| 1865 | 2124 | secreted acidic cysteine rich glycoprotein | 240.27 | 605640-606011 |
| 1902 | 2112 | olfactomedin-like 2B | 15.33 | 618054-618379 |
| 1934 | 2100 | heparin-binding EGF-like growth factor | 10.18 | 629091-629425 |
| 1990 | 2082 | protein S ( ) | 10.73 | 648173-648463 |
| 2065 | 2055 | integrin FG-GAP repeat containing 1 | 7.636 | 673176-673566 |
| 2088 | 2048 | ST3 β-galactoside-2,3-sialyltransferase 1 | 5.651 | 681105-681454 |
| 2109 | 2041 | disintegrin-like & metallopeptidase (reprolysin | 0.788 | 687923-688239 |
| | | type) with thrombospondin type 1 motif, 1 | | |
| 2140 | 2029 | colony stimulating factor 1 (macrophage) | 2.182 | 698431-698749 |
| 2440 | 1946 | arginyl aminopeptidase (aminopeptidase B) | 9.264 | 800159-800460 |
| 2474 | 1940 | epiregulin | 9.501 | 811533-811821 |
| 2477 | 1938 | complement component factor h | 1.484 | 812520-812875 |
| 2542 | 1922 | selenoprotein P, plasma, 1 | 49.03 | 835040-835364 |
| 2618 | 1903 | granulin | 165.89 | 860464-860761 |
| 2627 | 1901 | tubulointerstitial nephritis antigen-like 1 | 471.92 | 863337-863698 |
| 2667 | 1890 | family with sequence similarity 20, member C | 2.956 | 876909-877243 |
| 2698 | 1885 | insulin-like growth factor binding protein 4 | 45.48 | 887505-887820 |
| 2719 | 1879 | extracellular matrix protein 1 | 8.456 | 894665-894972 |
| 2722 | 1877 | calreticulin | 630.60 | 895691-896051 |
| 2724 | 1876 | cysteine rich protein 61 | 28.82 | 896415-896793 |
| 2755 | 1869 | tenascin XB | 0.354 | 907253-907581 |
| 2774 | 1862 | glucose-fructose oxidoreductase domain | 4.766 | 913662-913993 |
| | | containing 2 | | |
| 2782 | 1861 | procollagen C-endopeptidase enhancer 2 | 46.63 | 916348-916725 |
| 2820 | 1853 | biotinidase | 6.907 | 929397-929702 |
| 2866 | 1840 | milk fat globule-EGF factor 8 protein | 184.99 | 945014-945413 |
| 2890 | 1833 | coiled-coil domain containing 126 | 9.438 | 953382-953706 |
| 2960 | 1813 | elastin microfibril interfacer 1 | 5.563 | 977509-977878 |
| 2980 | 1806 | galactoside-binding lectin soluble 3 | 90.44 | 984430-984814 |
| 3067 | 1775 | fibroblast growth factor 7 | 1.663 | 1013719-1014044 |
| 3118 | 1765 | glucose phosphate isomerase 1 | 16.66 | 1031173-1031520 |
| 3129 | 1763 | matrix metallopeptidase 3 | 44.78 | 1034832-1035193 |
| 3242 | 1737 | arylsulfatase J | 2.374 | 1073837-1074138 |
| 3284 | 1723 | sushi-repeat-containing protein, X-linked 2 | 13.969 | 1087870-1088253 |
| 3296 | 1720 | suppression of tumorigenicity 14 | 2.378 | 1092011-1092357 |
| | | (colon carcinoma) | | |
| 3297 | 1719 | four jointed box 1 (Drosophila) | 3.328 | 1092358-1092750 |
| 3318 | 1714 | ependymin related protein 1 (zebrafish) | 16.418 | 1099390-1099766 |
| 3349 | 1706 | RIKEN cDNA 4930503L19 gene | 2.085 | 1109779-1110086 |
| 3370 | 1701 | mannosidase 2, B2 | 6.356 | 1116936-1117323 |
| 3410 | 1689 | neuroblastoma, suppression of tumorigenicity 1 | 73.672 | 1130855-1131122 |
| 3509 | 1667 | heparanase | 5.741 | 1163608-1163901 |
| 3517 | 1666 | aarF domain containing kinase 1 | 1.499 | 1166401-1166741 |
| 3565 | 1652 | HtrA serine peptidase 1 | 42.699 | 1182505-1182824 |
| 3639 | 1636 | chitinase domain containing 1 | 16.866 | 1207474-1207818 |
| 3673 | 1628 | corneodesmosin | 3.545 | 1218944-1219310 |
| 3727 | 1618 | vascular endothelial growth factor C | 10.284 | 1237351-1237686 |
| 3749 | 1612 | ADAMTS-like 4 | 2.67 | 1244700-1245081 |
| 3783 | 1602 | epidermal growth factor-containing fibulin-like | 6.911 | 1256425-1256734 |
| | | extracellular matrix protein 1 | | |
| 3809 | 1594 | matrix metallopeptidase 10 | 43.632 | 1265238-1265630 |
| 3879 | 1578 | aldolase A, fructose-bisphosphate | 476.31 | 1288654-1288987 |
| 3926 | 1567 | clusterin | 40.878 | 1304084-1304407 |
| 4064 | 1533 | phospholipid transfer protein | 39.57 | 1350158-1350474 |
| 4109 | 1521 | glycosylphosphatidylinositol specific | 0.591 | 1365026-1365348 |
| | | phospholipase D1 | | |
| 4177 | 1507 | RIKEN cDNA A130022J15 gene | 1.007 | 1387950-1388266 |
| 4188 | 1504 | EGF-containing fibulin-like extracellular | 45.43 | 1391741-1392104 |
| | | matrix protein 2 | | |
| 4234 | 1494 | WAP, FS, Ig, KU, & NTR-containing protein 1 | 1.307 | 1407418-1407713 |
| 4240 | 1493 | complement factor properdin | 2.075 | 1409395-1409692 |
| 4245 | 1492 | Ser (or Cys) peptidase inhibitor, clade I, member 1 | 0.687 | 1410934-1411281 |
| 4280 | 1485 | glutathione reductase | 6.516 | 1422793-1423122 |
| 4282 | 1485 | matrix metallopeptidase 12 | 15.393 | 1423446-1423812 |
| 4319 | 1476 | ST3 beta-galactoside-2,3-sialyltransferase 2 | 1.043 | 1435989-1436317 |
| 4345 | 1471 | procollagen C-endopeptidase enhancer protein | 38.334 | 1444649-1444973 |
| 4362 | 1468 | serum amyloid A-like 1 | 2.535 | 1450214-1450482 |
| 4405 | 1458 | tsukushin | 2.692 | 1464641-1464971 |
| 4410 | 1457 | sodium channel, nonvoltage-gated 1 | 0.749 | 1466293-1466624 |
| 4417 | 1456 | ADP-dependent glucokinase | 1.872 | 1468606-1468902 |
| 4513 | 1433 | leukemia inhibitory factor | 2.095 | 1499872-1500182 |
| 4538 | 1428 | RIKEN cDNA 3110057O12 gene | 0.612 | 1508213-1508566 |
| 4576 | 1420 | CD109 antigen | 0.579 | 1521122-1521452 |
| 4614 | 1413 | family with sequence similarity 3, member A | 24.923 | 1533979-1534266 |
| 4627 | 1408 | parathyroid hormone-like peptide | 4.769 | 1537818-1538138 |
| 4767 | 1376 | serine (or cysteine) peptidase inhibitor, | 20.015 | 1584786-1585074 |
| | | clade F, member 1 | | |
| 4772 | 1374 | annexin A2 | 701.66 | 1586334-1586631 |
| 4801 | 1368 | cysteine-rich with EGF-like domains 2 | 53.263 | 1596381-1596717 |
| 4834 | 1362 | hedgehog interacting protein-like 1 | 1.94 | 1607854-1608237 |
| 4843 | 1359 | laminin, γ 2 | 0.673 | 1610932-1611257 |
| 4846 | 1358 | family with sequence similarity 108, member A | 22.48 | 1611921-1612236 |
| 4847 | 1358 | secreted phosphoprotein 1 | 200.26 | 1612237-1612512 |
| 4878 | 1352 | C1q and tumor necrosis factor related protein 4 | 48.396 | 1622523-1622869 |
| 4923 | 1344 | Von Willebrand factor homolog | 0.168 | 1638235-1638612 |
| 4959 | 1336 | paraoxonase 2 | 17.99 | 1650552-1650935 |
| 4965 | 1332 | collagen, type III, 1 | 0.44 | 1652715-1653073 |
| 4993 | 1326 | collagen, type XVIII, 1 | 0.529 | 1662476-1662775 |
| 4995 | 1325 | Norrie disease (pseudoglioma) (human) | 2.955 | 1663144-1663508 |
| 5017 | 1320 | olfactomedin-like 3 | 1.465 | 1670554-1670828 |
| 5071 | 1308 | endonuclease domain containing 1 | 1.415 | 1688826-1689139 |
| 5100 | 1301 | sema domain, immunoglobulin domain (Ig), | 0.608 | 1698838-1699148 |
| | | short basic domain, secreted, (semaphorin) 3E | | |
| 5102 | 1300 | complement component (3b/4b) receptor 1-like | 36.058 | 1699537-1699891 |
| 5103 | 1300 | histocompatibility 2, D region locus 1 | 14.507 | 1699892-1699970 |
| 5145 | 1290 | dehydrogenase/reductase (SDR family) | 3.209 | 1714013-1714298 |
| | | member 13 | | |
| 5151 | 1288 | cytokine receptor-like factor 1 | 35.42 | 1715952-1716278 |
| 5183 | 1283 | acid phosphatase 6, lysophosphatidic | 4.044 | 1727109-1727397 |
| 5231 | 1274 | latent transforming growth factor β binding | 0.288 | 1743609-1743996 |
| | | protein 2 | | |
| 5233 | 1274 | histocompatibility 2, K1, K region | 12.62 | 1744314-1744510 |
| 5244 | 1272 | interleukin 4 receptor, | 1.087 | 1748021-1748398 |
| 5265 | 1268 | interleukin 33 | 27.994 | 1755091-1755384 |
| 5270 | 1267 | zona pellucida binding protein 2 | 8.813 | 1756658-1757006 |
| 5275 | 1265 | family with sequence similarity 3, member C | 6.069 | 1758518-1758803 |
| 5357 | 1245 | transforming growth factor, β 1 | 13.689 | 1787146-1787456 |
| 5390 | 1239 | N-acetylglucosamine-1-phosphotransferase, | 11.34 | 1799084-1799470 |
| | | γ subunit | | |
| 5400 | 1237 | cartilage associated protein | 24.359 | 1802419-1802805 |
| 5421 | 1232 | intercellular adhesion molecule 1 | 3.334 | 1809854-1810180 |
| 5428 | 1230 | calsyntenin 1 | 0.828 | 1812199-1812578 |
| 5435 | 1229 | meteorin, glial cell differentiation regulator-like | 5.487 | 1814631-1814930 |
| 5450 | 1225 | wingless-related MMTV integration site 7B | 0.932 | 1819882-1820264 |
| 5519 | 1207 | glucose-fructose oxidoreductase domain | 0.479 | 1844526-1844882 |
| | | containing 1 | | |
| 5520 | 1207 | carboxypeptidase X 1 (M14 family) | 0.795 | 1844883-1845160 |
| 5529 | 1205 | glutamyl aminopeptidase | 0.69 | 1847806-1848189 |
| 5537 | 1202 | angiopoietin-like 4 | 0.987 | 1850651-1851035 |
| 5550 | 1200 | a disintegrin and metallopeptidase domain 17 | 1.374 | 1855220-1855596 |
| 5556 | 1199 | dickkopf homolog 3 (Xenopus laevis) | 1.782 | 1857147-1857502 |
| 5644 | 1179 | complement component 3 | 0.472 | 1888266-1888655 |
| 5682 | 1170 | transforming growth factor, β receptor III | 6.658 | 1901807-1902171 |
| 5694 | 1168 | vascular endothelial growth factor B | 11.401 | 1906017-1906367 |
| 5710 | 1164 | decorin | 1.4 | 1911705-1912079 |
| 5716 | 1164 | cofilin 1, non-muscle | 107.83 | 1914036-1914356 |
| 5718 | 1163 | lysyl oxidase-like 2 | 0.322 | 1914742-1915076 |
| 5735 | 1160 | thioredoxin domain containing 16 | 0.533 | 1920932-1921309 |
| 5752 | 1156 | capping protein (actin filament), gelsolin-like | 62.723 | 1927144-1927507 |
| 5783 | 1148 | lectin, galactose binding, soluble 9 | 12.269 | 1938395-1938769 |
| 5792 | 1147 | matrix metallopeptidase 13 | 0.724 | 1941794-1942151 |
| 5800 | 1145 | multiple coagulation factor deficiency 2 | 5.202 | 1944542-1944919 |
| 5810 | 1144 | Kazal-type serine peptidase inhibitor domain 1 | 37.259 | 1948146-1948458 |
| 5841 | 1138 | collagen, type V, 2 | 0.225 | 1959286-1959679 |
| 5854 | 1136 | caspase 1 | 2.306 | 1964106-1964500 |
| 5872 | 1132 | γ-glutamyl hydrolase | 9.842 | 1970781-1971062 |
| 5964 | 1111 | colony stimulating factor 3 (granulocyte) | 2.413 | 2004485-2004820 |
| 5967 | 1110 | cellular repressor of E1A-stimulated genes 1 | 3.396 | 2005583-2005881 |
| 6004 | 1100 | RIKEN cDNA 1600012H06 gene | 1.469 | 2018789-2019169 |
| 6033 | 1095 | protease, serine 27 | 3.375 | 2029351-2029692 |
| 6059 | 1090 | torsin family 2, member A | 4.118 | 2038737-2039067 |
| 6069 | 1087 | DDRGK domain containing 1 | 25.9 | 2042411-2042776 |
| 6177 | 1063 | dehydrogenase/reductase (SDR family) member 11 | 3.811 | 2081334-2081729 |
| 6185 | 1062 | aminoacyl tRNA synthetase complex- | 33.092 | 2084323-2084687 |
| | | interacting multifunctional protein 1 | | |
| 6208 | 1056 | coiled-coil domain containing 134 | 4.556 | 2092810-2093167 |
| 6234 | 1050 | plasminogen activator, urokinase receptor | 78.786 | 2102477-2102872 |
| 6237 | 1049 | phospholipase A2, group XV | 1.496 | 2103576-2103969 |
| 6273 | 1039 | nerve growth factor | 9.393 | 2115896-2116286 |
| 6276 | 1038 | wingless-related MMTV integration site 4 | 32.674 | 2116955-2117340 |
| 6296 | 1034 | kelch-like 11 (Drosophila) | 0.425 | 2124257-2124635 |
| 6328 | 1028 | hydroxysteroid (17-β) dehydrogenase 11 | 4.421 | 2135382-2135767 |
| 6334 | 1028 | chemokine (C—X—C motif) ligand 12 | 0.641 | 2137589-2137972 |
| 6363 | 1021 | netrin 4 | 3.366 | 2148005-2148402 |
| 6385 | 1017 | follistatin | 0.853 | 2155919-2156270 |
| 6412 | 1009 | GLI pathogenesis-related 2 | 2.074 | 2165641-2165996 |
| 6457 | 998 | ecto-NOX disulfide-thiol exchanger 1 | 3.002 | 2181525-2181862 |
| 6493 | 989 | collagen, type VII, 1 | 0.344 | 2194670-2194969 |
| 6627 | 964 | meteorin, glial cell differentiation regulator | 3.641 | 2241580-2241948 |
| 6665 | 955 | hyaluronic acid binding protein 4 | 2.739 | 2255107-2255429 |
| 6773 | 932 | inhibin β-B | 1.597 | 2292605-2292959 |
| 6787 | 928 | wingless-related MMTV integration site 5B | 0.458 | 2297590-2297892 |
| 6816 | 921 | peroxidasin homolog (Drosophila) | 0.334 | 2307638-2308007 |
| 6819 | 920 | integrin 2b | 0.686 | 2308648-2308928 |
| 6830 | 918 | interleukin 19 | 4.282 | 2312386-2312719 |
| 6900 | 903 | phospholipase A2, group XIIA | 11.576 | 2335117-2335473 |
| 6950 | 893 | angiogenic factor with G patch and FHA | 0.281 | 2351743-2352058 |
| | | domains 1 | | |
| 6964 | 889 | Niemann Pick type C2 | 40.486 | 2356243-2356636 |
| 6974 | 887 | apolipoprotein A-I binding protein | 13.178 | 2359569-2359941 |
| 7015 | 877 | TNF (ligand) superfamily, member 12 | 4.328 | 2373485-2373776 |
| 7019 | 876 | Cys rich transmembrane BMP regulator 1 | 0.287 | 2374809-2375187 |
| | | (chordin like) | | |
| 7021 | 875 | matrilin 4 | 7.832 | 2375566-2375930 |
| 7022 | 875 | artemin | 2.794 | 2375931-2376296 |
| 7109 | 858 | matrix metallopeptidase 19 | 0.305 | 2404764-2405144 |
| 7125 | 853 | profilin 1 | 11.177 | 2410108-2410492 |
| 7126 | 852 | vasohibin 1 | 0.138 | 2410493-2410795 |
| 7142 | 849 | Parkinson disease 7 domain containing 1 | 1.935 | 2415737-2416107 |
| 7156 | 846 | intercellular adhesion molecule 4, Landsteiner- | 5.958 | 2420123-2420515 |
| | | Wiener blood group | | |
| 7158 | 845 | c-fos induced growth factor | 3.445 | 2420809-2421101 |
| 7185 | 839 | leucine-rich repeats and calponin homology | 0.206 | 2429731-2430059 |
| | | (CH) domain containing 3 | | |
| 7192 | 839 | VGF nerve growth factor inducible | 0.371 | 2432094-2432431 |
| 7199 | 838 | transforming growth factor, β 3 | 1.124 | 2434410-2434754 |
| 7223 | 833 | chemokine (C—X—C motif) ligand 1 | 3.826 | 2442608-2443003 |
| 7234 | 830 | WNT1 inducible signaling pathway protein 2 | 1.032 | 2446311-2446606 |
| 7259 | 824 | leucine-rich repeat LGI family, member 4 | 0.356 | 2454637-2454993 |
| 7279 | 817 | follistatin-like 1 | 0.406 | 2460885-2461283 |
| 7305 | 810 | tissue factor pathway inhibitor | 4.848 | 2469295-2469576 |
| 7328 | 804 | inhibin | 0.548 | 2477026-2477404 |
| 7360 | 796 | placental specific protein 1 | 2.395 | 2487553-2487920 |
| 7380 | 793 | stromal cell derived factor 2 | 6.558 | 2494318-2494652 |
| 7450 | 775 | FMS-like tyrosine kinase 3 ligand | 4.868 | 2517516-2517899 |
| 7454 | 774 | platelet derived growth factor, | 4.859 | 2518844-2519200 |
| 7469 | 770 | CD1d1 antigen | 0.505 | 2523514-2523656 |
| 7475 | 769 | tissue inhibitor of metalloproteinase 1 | 42.275 | 2525246-2525550 |
| 7484 | 767 | UDP-Gal:betaGlcNAc β 1,4- | 0.387 | 2528454-2528763 |
| | | galactosyltransferase, polypeptide 1 | | |
| 7624 | 733 | sodium channel, nonvoltage-gated 1 β | 0.301 | 2574019-2574393 |
| 7628 | 732 | proline-rich Gla (G-carboxyglutamic acid) | 1.115 | 2575046-2575364 |
| | | polypeptide 2 | | |
| 7658 | 724 | hyaluronan and proteoglycan link protein 4 | 0.319 | 2584861-2585169 |
| 7676 | 720 | chemokine (C-C motif) ligand 2 | 14.55 | 2590794-2591157 |
| 7707 | 713 | intelectin 1 (galactofuranose binding) | 1.888 | 2601763-2602070 |
| 7726 | 708 | interleukin 17F | 3.058 | 2607930-2608234 |
| 7758 | 700 | bone morphogenetic protein 2 | 0.343 | 2618776-2619161 |
| 7770 | 697 | olfactomedin 2 | 0.593 | 2622919-2623236 |
| 7789 | 692 | collagen, type VIII, 1 | 0.136 | 2629576-2629946 |
| 7810 | 688 | mesencephalic astrocyte-derived | 3.849 | 2636612-2636951 |
| | | neurotrophic factor | | |
| 7820 | 685 | integrin X | 0.229 | 2639993-2640227 |
| 7827 | 683 | versican | 0.055 | 2642303-2642596 |
| 7874 | 666 | CD1d2 antigen | 0.935 | 2658252-2658336 |
| 7903 | 658 | interleukin 1 receptor accessory protein | 0.254 | 2667913-2668256 |
| 7929 | 651 | interleukin 23, subunit p19 | 0.852 | 2676772-2677097 |
| 7935 | 649 | follistatin-like 3 | 0.427 | 2678648-2679041 |
| 7938 | 649 | stanniocalcin 2 | 0.821 | 2679803-2680201 |
| 7940 | 648 | matrix metallopeptidase 17 | 0.224 | 2680510-2680844 |
| 7947 | 646 | wingless-type MMTV integration site 9A | 0.20 | 2682871-2683194 |
| 7979 | 638 | protease, serine, 8 (prostasin) | 0.479 | 2693206-2693562 |
| 8062 | 610 | fibroblast growth factor 18 | 1.273 | 2720721-2721030 |
| 8066 | 610 | ribonuclease, RNase A family 4 | 9.649 | 2721991-2722365 |
| 8108 | 598 | thymosin, β 4, X chromosome | 24.043 | 2734875-2735269 |
| 8119 | 595 | serglycin | 9.946 | 2738723-2739031 |
| 8138 | 590 | RIKEN cDNA 1700040I03 gene | 2.322 | 2744620-2744956 |
| 8146 | 588 | cardiotrophin-like cytokine factor 1 | 1.757 | 2747178-2747573 |
| 8167 | 584 | agouti related protein | 1.444 | 2753704-2754040 |
| 8218 | 570 | interleukin 18 | 2.856 | 2769797-2770097 |
| 8226 | 568 | DNA segment, Chr 17, Wayne State | 3.239 | 2772236-2772535 |
| | | University 104, expressed | | |
| 8244 | 562 | interleukin 1 receptor-like 1 | 0.299 | 2777898-2778255 |
| 8255 | 558 | matrix metallopeptidase 24 | 6.978 | 2781318-2781710 |
| 8257 | 558 | elastin microfibril interfacer 3 | 0.17 | 2782095-2782379 |
| 8303 | 547 | C1q and tumor necrosis factor related protein 1 | 0.218 | 2797989-2798315 |
| 8304 | 546 | macrophage migration inhibitory factor | 43.469 | 2798316-2798434 |
| 8332 | 540 | twisted gastrulation homolog 1 (Drosophila) | 0.318 | 2807636-2808031 |
| 8345 | 536 | Fas (TNF receptor superfamily member 6) | 0.501 | 2812206-2812506 |
| 8385 | 524 | natriuretic peptide precursor type B | 2.217 | 2825789-2826134 |
| 8387 | 523 | suprabasin | 2.479 | 2826504-2826901 |
| 8394 | 521 | cystatin C | 17.163 | 2828994-2829393 |
| 8410 | 516 | sema domain, immunoglobulin domain (Ig), | 0.212 | 2834784-2835155 |
| | | short basic domain, secreted, (semaphorin) 3C | | |
| 8440 | 510 | a disintegrin-like and metallopeptidase | 0.139 | 2845165-2845528 |
| | | (reprolysin type) with thrombospondin type 1 | | |
| | | motif, 4 | | |
| 8500 | 495 | natriuretic peptide precursor type A | 1.563 | 2864212-2864568 |
| 8504 | 494 | chemokine (C—X—C motif) ligand 10 | 1.586 | 2865648-2866015 |
| 8531 | 490 | interleukin 15 | 1.901 | 2874576-2874952 |
| 8553 | 485 | interleukin 11 | 0.384 | 2881854-2882091 |
| 8560 | 485 | retinoic acid receptor responder (tazarotene | 0.687 | 2883778-2884132 |
| | | induced) 2 | | |
| 8581 | 480 | lectin, galactose binding, soluble 1 | 282.39 | 2890379-2890745 |
| 8597 | 477 | mannan-binding lectin serine peptidase 2 | 0.156 | 2896069-2896411 |
| 8647 | 467 | RIKEN cDNA 2300009A05 gene | 0.768 | 2912945-2913330 |
| 8696 | 459 | CSF 2 (granulocyte-macrophage) | 1.109 | 2928757-2929061 |
| 8697 | 459 | interleukin 18 binding protein | 1.553 | 2929062-2929418 |
| 8698 | 459 | prenylcysteine oxidase 1 like | 0.228 | 2929419-2929743 |
| 8708 | 456 | apolipoprotein O-like | 0.456 | 2932503-2932836 |
| 8713 | 455 | neuron derived neurotrophic factor | 1.137 | 2933997-2934318 |
| 8746 | 450 | TNF receptor superfamily, member 4 | 0.392 | 2944708-2945036 |
| 8753 | 449 | sparc/osteonectin, cwcv & kazal-like domains | 0.172 | 2946657-2946988 |
| | | proteoglycan 1 | | |
| 8756 | 449 | integrin 1 | 0.15 | 2947656-2948022 |
| 8777 | 444 | laminin, 2 | 0.046 | 2954307-2954650 |
| 8784 | 443 | thyroglobulin | 0.076 | 2956549-2956869 |
| 8821 | 437 | apolipoprotein M | 0.598 | 2967624-2967944 |
| 8871 | 423 | spondin 2, extracellular matrix protein | 0.189 | 2982359-2982686 |
| 8876 | 422 | elastin microfibril interfacer 2 | 0.11 | 2983901-2984203 |
| 8916 | 414 | anti-Mullerian hormone | 0.248 | 2995308-2995592 |
| 8935 | 409 | carboxypeptidase N, polypeptide 1 | 0.233 | 3000705-3001032 |
| 8945 | 407 | insulin-like growth factor binding protein 6 | 0.548 | 3003421-3003704 |
| 9021 | 387 | hemopexin | 0.262 | 3023816-3024122 |
| 9063 | 374 | periostin, osteoblast specific factor | 0.118 | 3034618-3034877 |
| 9064 | 373 | complement component 8, γ polypeptide | 0.685 | 3034878-3035143 |
| 9079 | 370 | neuregulin 3 | 0.146 | 3038641-3038935 |
| 9116 | 361 | RIKEN cDNA 1190002N15 gene | 0.094 | 3047961-3048223 |
| 9120 | 360 | adrenomedullin | 0.331 | 3048901-3049164 |
| 9131 | 357 | apolipoprotein A-II | 1.494 | 3051648-3051933 |
| 9136 | 356 | nonagouti | 0.963 | 3052970-3053198 |
| 9151 | 352 | TNF receptor superfamily, member 22 | 0.691 | 3056380-3056639 |
| 9164 | 348 | TNF (ligand) superfamily, member 11 | 0.157 | 3058993-3059213 |
| 9185 | 344 | Serine (or Cys) peptidase inhibitor, clade C | 0.158 | 3063585-3063840 |
| | | (antithrombin), member 1 | | |
| 9207 | 339 | RIKEN cDNA A430110N23 gene | 0.132 | 3068647-3068843 |
| 9212 | 339 | canopy 4 homolog (zebrafish) | 0.335 | 3069460-3069696 |
| 9230 | 335 | regenerating islet-derived 3 γ | 0.43 | 3073532-3073815 |
| 9244 | 331 | arylsulfatase K | 0.177 | 3076784-3077031 |
| 9267 | 324 | cerebral dopamine neurotrophic factor | 0.109 | 3081521-3081786 |
| 9274 | 322 | bone morphogenetic protein 6 | 0.219 | 3083187-3083415 |
| 9290 | 319 | carboxypeptidase B2 (plasma) | 0.216 | 3086591-3086854 |
| 9293 | 318 | deoxyribonuclease 1-like 2 | 0.409 | 3087405-3087662 |
| 9295 | 318 | apolipoprotein H | 0.493 | 3087876-3088127 |
| 9307 | 312 | growth hormone receptor | 0.289 | 3090523-3090733 |
| 9325 | 307 | transglutaminase 4 (prostate) | 0.112 | 3094562-3094802 |
| 9363 | 296 | oncostatin M | 0.135 | 3102482-3102721 |
| 9366 | 295 | osteomodulin | 0.169 | 3103083-3103312 |
| 9367 | 295 | Fc receptor, IgG, low affinity IIb | 0.189 | 3103313-3103351 |
| 9368 | 295 | DAN domain family, member 5 | 0.189 | 3103352-3103518 |
| 9375 | 293 | antigen p97 (melanoma associated) identified | 0.073 | 3104582-3104752 |
| | | by mAbs 133.2 and 96.5 | | |
| 9394 | 285 | carboxylesterase 7 | 0.166 | 3108135-3108339 |
| 9402 | 282 | ISG15 ubiquitin-like modifier | 1.263 | 3109784-3109974 |
| 9403 | 282 | RIKEN cDNA 4930486L24 gene | 0.203 | 3109975-3110173 |
| 9404 | 281 | transmembrane protein 25 | 0.122 | 3110174-3110389 |
| 9412 | 278 | cDNA sequence BC039632 | 0.114 | 3111726-3111929 |
| 9431 | 270 | GLI pathogenesis-related 1 (glioma) | 0.512 | 3115200-3115432 |
| 9461 | 260 | carbonic anhydrase 15 | 0.231 | 3120401-3120588 |
| 9518 | 237 | cytotoxic T lymphocyte-associated protein 2 | 0.174 | 3129312-3129456 |
| 9536 | 233 | laminin γ 3 | 0.04 | 3131997-3132159 |
| 9560 | 222 | RIKEN cDNA 1110058L19 gene | 0.33 | 3135368-3135519 |
| 9593 | 210 | family with sequence similarity 20, member B | 0.05 | 3139182-3139331 |
| 9604 | 205 | sparc/osteonectin, cwcv and kazal-like | 0.313 | 3140413-3140532 |
| | | domains proteoglycan 2 | | |
| 9611 | 202 | chemokine (C-C motif) ligand 9 | 0.268 | 3141032-3141071 |
| 9654 | 185 | cerebellin 3 precursor protein | 0.051 | 3144853-3144886 |
| 9673 | 174 | cellular repressor of E1A-stimulated genes 2 | 0.11 | 3146685-3146736 |
| 9694 | 166 | histocompatibility 2, M region locus 3 | 0.309 | NA-NA |
| 9720 | 149 | chemokine (C—X—C motif) ligand 3 | 0.148 | 3149776-3149850 |
| 9740 | 139 | β cellulin, epidermal growth factor family | 0.073 | 3150839-3150877 |
| | | member | | |
| 9742 | 139 | hyaluronoglucosaminidase 1 | 0.064 | 3150976-3151021 |
| 9756 | 131 | glutathione peroxidase 3 | 0.087 | 3151589-3151685 |
| 3157149 | 488 | tectorin β | 0.18 | 3161121-3161220 |
| 3157152 | 479 | angiogenin, ribonuclease, RNase A family, 5 | 0.895 | 3217891-3217990 |
| 3157165 | 234 | surfactant associated protein D | 0.176 | 3266005-3266104 |
| 3157173 | 1664 | transcobalamin 2 | 5.78 | 3266205-3266304 |
| 3157204 | 1498 | NA | 0.661 | 3239917-3240016 |
| 3157207 | 463 | epiphycan | 0.269 | 3166484-3166583 |
| 3157217 | 384 | thrombospondin, type I, domain containing 4 | 0.044 | 3224191-3224290 |
| 3157225 | 705 | renalase, FAD-dependent amine oxidase | 2.03 | 3245517-3245616 |
| 3157231 | 468 | macrophage stimulating 1 (hepatocyte growth | 0.205 | 3240817-3240916 |
| | | factor-like) | | |
| 3157234 | 711 | neuregulin 4 | 1.009 | 3219591-3219690 |
| 3157276 | 1883 | cell adhesion molecule w/ homology to L1CAM | 0.289 | 3252517-3252616 |
| 3157279 | 427 | ectonucleotide pyrophosphatase/ | 0.153 | 3182184-3182283 |
| | | phosphodiesterase 3 | | |
| 3157283 | 323 | NA | 0.115 | 3279349-3279448 |
| 3157286 | 416 | C1q-like 3 | 0.132 | 3208858-3208957 |
| 3157290 | 388 | carbonic anhydrase 11 | 0.267 | 3238117-3238216 |
| 3157305 | 665 | angiomotin | 0.309 | 3173084-3173183 |
| 3157331 | 711 | isthmin 1 homolog (zebrafish) | 0.244 | 3172584-3172683 |
| 3157343 | 370 | predicted gene 1019 | 0.391 | 3193971-3194070 |
| 3157352 | 311 | killer cell lectin-like receptor, subfamily D, | 0.43 | 3221191-3221290 |
| | | member 1 | | |
| 3157362 | 1350 | immunoglobulin superfamily containing | 2.61 | 3279049-3279148 |
| | | leucine-rich repeat | | |
| 3157366 | 450 | angiotensinogen (serpin peptidase inhibitor, | 0.242 | 3260305-3260404 |
| | | clade A, member 8) | | |
| 3157368 | 373 | interleukin 16 | 0.075 | 3232717-3232816 |
| 3157372 | 584 | lipase, family member N | 0.389 | 3192971-3193070 |
| 3157373 | 339 | angiopoietin 4 | 0.222 | 3239317-3239416 |
| 3157414 | 285 | glycine receptor, β subunit | 0.096 | 3187971-3188070 |
| 3157415 | 568 | integrin 6 | 0.213 | 3201597-3201696 |
| 3157422 | 1431 | G protein-coupled receptor 125 | 1.185 | 3236817-3236916 |
| 3157455 | 494 | dehydrogenase/reductase (SDR family) member 7C | 0.832 | 3255005-3255104 |
| 3157459 | 250 | chemokine (C-C motif) ligand 11 | 0.299 | 3199071-3199170 |
| 3157475 | 403 | paraoxonase 3 | 0.226 | 3268005-3268104 |
| 3157481 | 804 | follistatin-like 4 | 0.303 | 3183884-3183983 |
| 3157491 | 639 | G protein-coupled receptor 98 | 0.033 | 3188771-3188870 |
| 3157500 | 458 | seizure related gene 6 | 0.114 | 3189371-3189470 |
| 3157503 | 787 | pentraxin related gene | 1.801 | 3175884-3175983 |
| 3157510 | 700 | secretory leukocyte peptidase inhibitor | 7.778 | 3248817-3248916 |
| 3157516 | 361 | roundabout homolog 4 (Drosophila) | 0.098 | 3164884-3164983 |
| 3157520 | 492 | complement component 1, r subcomponent-like | 0.264 | 3224791-3224890 |
| 3157537 | 234 | mucin 13, epithelial transmembrane | 0.08 | 3203297-3203396 |
| 3157558 | 742 | chemokine (C-C motif) ligand 7 | 6.395 | 3279849-3279948 |
| 3157590 | 520 | interleukin 13 receptor, 2 | 0.336 | 3213558-3213657 |
| 3157601 | 267 | fukutin related protein | 0.095 | 3212358-3212457 |
| 3157619 | 289 | fin bud initiation factor homolog (zebrafish) | 0.14 | 3185471-3185570 |
| 3157676 | 961 | extracellular matrix protein 2, female organ | 0.343 | 3256205-3256304 |
| | | and adipocyte specific | | |
| 3157717 | 366 | Fras1 related extracellular matrix protein 1 | 0.039 | 3271296-3271395 |
| 3157721 | 413 | EGF-like module containing, mucin-like, | 0.249 | 3218391-3218490 |
| | | hormone receptor-like sequence 1 | | |
| 3157729 | 356 | tectorin | 0.049 | 3257705-3257804 |
| 3157760 | 967 | interleukin 7 receptor | 0.428 | 3216691-3216790 |
| 3157775 | 648 | multiple EGF-like-domains 6 | 0.147 | 3174384-3174483 |
| 3157796 | 402 | secreted phosphoprotein 2 | 0.468 | 3270196-3270295 |
| 3157845 | 837 | mast cell protease 8 | 4.869 | 3206558-3206657 |
| 3157850 | 577 | collagen, type XV, 1 | 0.108 | 3250617-3250716 |
| 3157858 | 323 | apolipoprotein E | 0.255 | 3172384-3172483 |
| 3157868 | 306 | cathelicidin antimicrobial peptide | 0.513 | 3234517-3234616 |
| 3157885 | 1542 | sema domain, immunoglobulin domain (Ig), | 0.705 | 3168184-3168283 |
| | | short basic domain, secreted, (semaphorin) 3A | | |
| 3157898 | 422 | disintegrin-like and metallopeptidase (reprolysin | 0.115 | 3193471-3193570 |
| | | type) with thrombospondin type 1 motif, 15 | | |
| 3157902 | 1558 | fibrillin 1 | 0.197 | 3211258-3211357 |
| 3157936 | 2200 | laminin, 3 | 0.41 | 3160721-3160820 |
| 3157937 | 697 | collagen, type XVII, 1 | 0.131 | 3163384-3163483 |
| 3157938 | 372 | secretagogin, EF-hand calcium binding protein | 0.26 | 3258005-3258104 |
| 3157949 | 477 | protein C | 0.42 | 3271796-3271895 |
| 3157974 | 2507 | thrombospondin 2 | 1.595 | 3265805-3265904 |
| 3157977 | 1031 | interleukin 7 | 0.642 | 3242917-3243016 |
| 3158019 | 362 | ABO blood group (transferase A, 1-3-N- | 0.204 | 3185571-3185670 |
| | | acetylgalactosaminyltransferase, transferase B, | | |
| | | 1-3-galactosyltransferase) | | |
| 3158024 | 541 | immunoglobulin superfamily, member 10 | 0.078 | 3194171-3194270 |
| 3158034 | 923 | HtrA serine peptidase 3 | 0.583 | 3258505-3258604 |
| 3158038 | 176 | Fc receptor, IgE, high affinity I, γpolypeptide | 0.258 | 3201197-3201296 |
| 3158050 | 435 | lumican | 0.209 | 3262905-3263004 |
| 3158075 | 480 | potassium inwardly-rectifying channel, | 0.297 | 3169184-3169283 |
| | | subfamily J, member 3 | | |
| 3158077 | 496 | fibulin 5 | 0.198 | 3239017-3239116 |
| 3158079 | 282 | expressed sequence AI462493 | 0.577 | 3210858-3210957 |
| 3158107 | 484 | scavenger receptor cysteine rich domain | 0.181 | 3161821-3161920 |
| | | containing, group B (4 domains) | | |
| 3158135 | 418 | mannan-binding lectin serine peptidase 1 | 0.152 | 3282249-3282348 |
| 3158185 | 485 | interleukin 1 family, member 9 | 2.527 | 3241217-3241316 |
| 3158191 | 197 | dermatopontin | 0.125 | 3210958-3211057 |
| 3158201 | 416 | matrix metallopeptidase 21 | 0.224 | 3195471-3195570 |
| 3158209 | 1954 | fibroblast growth factor receptor 2 | 2.109 | 3207458-3207557 |
| 3158212 | 2457 | RIKEN cDNA 1300010F03 gene | 0.56 | 3182084-3182183 |
| 3158227 | 235 | bactericidal/permeability-increasing protein-like 2 | 0.101 | 3160521-3160620 |
| 3158236 | 1428 | R-spondin 3 homolog (Xenopus laevis) | 0.883 | 3261305-3261404 |
| 3158246 | 338 | coagulation factor VII | 0.181 | 3207558-3207657 |
| 3158249 | 442 | amylase 1, salivary | 0.247 | 3203097-3203196 |
| 3158274 | 393 | C-type lectin domain family 18, member A | 0.214 | 3219791-3219890 |
| 3158294 | 648 | matrix metallopeptidase 2 | 0.413 | 3214291-3214390 |
| 3158295 | 426 | stratifin | 0.681 | 3216091-3216190 |
| 3158307 | 369 | placental growth factor | 0.923 | 3227817-3227916 |
| 3158309 | 408 | adiponectin, C1Q and collagen domain containing | 0.331 | 3225317-3225416 |
| 3158310 | 262 | neuropeptide B | 0.483 | 3278149-3278248 |
| 3158331 | 982 | NEL-like 1 (chicken) | 0.565 | 3163221-3163320 |
| 3158365 | 431 | complement component factor i | 0.209 | 3178584-3178683 |
| 3158373 | 246 | pyroglutamylated RFamide peptide | 0.172 | 3209458-3209557 |
| 3158381 | 762 | CD24a antigen | 0.906 | 3245917-3246016 |
| 3158387 | 364 | ladinin | 0.193 | 3193271-3193370 |
| 3158415 | 552 | growth differentiation factor 11 | 0.45 | 3178384-3178483 |
| 3158419 | 1567 | NA | 1.244 | 3273596-3273695 |

TABLE 13
Cell cycle/Cell Division (Chinese hamster)

| SEQ | | | Avg | siRNA |
|---|---|---|---|---|
| ID NO: | consL | Description | Cov | SEQ ID NOs: |
| 1 | 7293 | ubiquitin specific peptidase 9, X chromosome | 6.127 | 9772-10147 |
| 19 | 4458 | platelet-activating factor acetylhydrolase, | 4.915 | 15430-15711 |
| | | isoform 1b, subunit 1 | | |
| 25 | 4353 | PDS5, regulator of cohesion maintenance, | 2.006 | 17099-17460 |
| | | homolog B (S. cerevisiae) | | |
| 81 | 3902 | integrin β 1 (fibronectin receptor β) | 126.69 | 35564-35891 |
| 126 | 3635 | E2F transcription factor 3 | 7.133 | 50121-50455 |
| 146 | 3553 | microtubule-actin crosslinking factor 1 | 3.329 | 56027-56372 |
| 149 | 3549 | stromal antigen 1 | 5.503 | 56906-57146 |
| 189 | 3384 | phosphatase and tensin homolog | 0.633 | 69091-69404 |
| 214 | 3308 | microtubule-associated protein, RP/EB | 9.685 | 76455-76767 |
| | | family, member 2 | | |
| 236 | 3232 | non-SMC condensin II complex, subunit D3 | 5.339 | 83095-83338 |
| 239 | 3230 | septin 11 | 14.203 | 83878-84130 |
| 266 | 3179 | vascular endothelial growth factor A | 18.713 | 92246-92594 |
| 287 | 3132 | splicing factor 1 | 10.149 | 98068-98328 |
| 304 | 3108 | Nipped-B homolog (Drosophila) | 1.896 | 103144-103477 |
| 317 | 3089 | cytoskeleton associated protein 5 | 5.989 | 106729-106971 |
| 345 | 3034 | glycogen synthase kinase 3 β | 0.647 | 114424-114743 |
| 375 | 2989 | RAD21 homolog (S. pombe) | 34.322 | 123260-123508 |
| 378 | 2983 | tousled-like kinase 1 | 3.811 | 124295-124551 |
| 382 | 2979 | breakpoint cluster region | 3.754 | 125289-125540 |
| 384 | 2977 | transcriptional regulator, SIN3A (yeast) | 3.56 | 125791-126119 |
| 426 | 2925 | stromal antigen 2 | 1.018 | 137619-137852 |
| 431 | 2919 | Tia1 cytotoxic granule-associated RNA | 12.569 | 139041-139241 |
| | | binding protein-like 1 | | |
| 432 | 2919 | cyclin D1 | 18.856 | 139242-139629 |
| 451 | 2894 | kinetochore associated 1 | 2.501 | 144746-145029 |
| 477 | 2865 | spindlin 1 | 18.581 | 151421-151677 |
| 486 | 2857 | anaphase promoting complex subunit 1 | 2.309 | 154085-154328 |
| 510 | 2835 | calcium/calmodulin-dependent protein kinase II γ | 4.887 | 161048-161267 |
| 528 | 2814 | spastin | 4.005 | 166072-166288 |
| 540 | 2799 | signal transducer & activator of transcription 5B | 1.323 | 169415-169753 |
| 549 | 2785 | AT hook containing transcription factor 1 | 2.992 | 172063-172296 |
| 573 | 2763 | calmodulin 1 | 15.152 | 178775-179029 |
| 589 | 2746 | nuclear protein in the AT region | 2.695 | 183475-183690 |
| 644 | 2703 | mitogen-activated protein kinase 6 | 18.977 | 200294-200550 |
| 658 | 2692 | structural maintenace of chromosomes 3 | 18.331 | 204131-204513 |
| 662 | 2689 | calcium/calmodulin-dependent protein | 5.415 | 205498-205717 |
| | | kinase II, δ | | |
| 689 | 2670 | budding uninhibited by benzimidazoles 1 | 3.768 | 213750-213996 |
| | | homolog (S. cerevisiae) | | |
| 745 | 2630 | minichromosome maintenance deficient 6 | 38.269 | 230817-231043 |
| | | (MIS5 homolog, yeast) | | |
| 800 | 2590 | TAF1 RNA polymerase II, TATA box | 1.877 | 247696-248086 |
| | | binding protein (TBP)-associated factor | | |
| 811 | 2582 | ajuba | 12.735 | 251195-251502 |
| 825 | 2573 | amyloid β (A4) precursor protein | 165.22 | 255412-255644 |
| 838 | 2566 | anaphase promoting complex subunit 4 | 10.429 | 259583-259826 |
| 866 | 2552 | timeless homolog (Drosophila) | 1.453 | 267981-268365 |
| 873 | 2550 | cyclin G associated kinase | 4.774 | 270072-270372 |
| 885 | 2543 | platelet-derived growth factor, C polypeptide | 3.586 | 273882-274243 |
| 889 | 2543 | katanin p80 (WD40-containing) subunit B 1 | 12.112 | 275290-275634 |
| 891 | 2542 | RB1-inducible coiled-coil 1 | 2.069 | 275944-276175 |
| 898 | 2540 | kinesin family member 20B | 10.559 | 278267-278603 |
| 899 | 2538 | transformation related protein 53 binding | 2.893 | 278604-278960 |
| | | protein 2 | | |
| 905 | 2536 | ADP-ribosylation factor-like 8B | 2.122 | 280457-280707 |
| 913 | 2532 | proteaseome (prosome, macropain) 28 subunit, 3 | 21.397 | 283197-283568 |
| 965 | 2501 | ubiquitin specific peptidase 16 | 11.237 | 300334-300663 |
| 990 | 2488 | ubiquitin-conjugating enzyme E2I | 38.98 | 308789-309160 |
| 1006 | 2477 | large tumor suppressor 2 | 3.379 | 314156-314545 |
| 1009 | 2476 | transcription factor Dp 2 | 2.614 | 315253-315631 |
| 1051 | 2450 | anaphase-promoting complex subunit 5 | 60.895 | 329249-329648 |
| 1053 | 2449 | polycystic kidney disease 1 homolog | 1.249 | 330038-330429 |
| 1062 | 2447 | septin 2 | 12.767 | 333080-333462 |
| 1068 | 2441 | chromatin assembly factor 1, subunit | 6.127 | 334746-335135 |
| | | A (p150) | | |
| 1070 | 2440 | promyelocytic leukemia | 1.141 | 335490-335874 |
| 1082 | 2434 | tousled-like kinase 2 (Arabidopsis) | 5.586 | 339541-339778 |
| 1091 | 2431 | ligase I, DNA, ATP-dependent | 14.03 | 342515-342854 |
| 1102 | 2427 | CTF18, chromosome transmission fidelity | 3.974 | 346257-346598 |
| | | factor 18 homolog (S. cerevisiae) | | |
| 1103 | 2426 | dystonin | 1.863 | 346599-346975 |
| 1188 | 2387 | WEE 1 homolog 1 (S. pombe) | 5.458 | 375593-375982 |
| 1208 | 2379 | CDC 14 cell division cycle 14 homolog A | 2.141 | 381807-382191 |
| | | (S. cerevisiae) | | |
| 1247 | 2359 | microtubule-associated protein, RP/EB | 18.63 | 394632-394981 |
| | | family, member 1 | | |
| 1255 | 2354 | centrosomal protein 110 | 0.814 | 397494-397774 |
| 1261 | 2353 | ligase III, DNA, ATP-dependent | 1.44 | 399254-399624 |
| 1321 | 2325 | beta-transducin repeat containing protein | 2.152 | 419725-419957 |
| 1327 | 2324 | centrosomal protein 55 | 19.363 | 421520-421872 |
| 1329 | 2323 | adenomatosis polyposis coli | 0.997 | 422123-422508 |
| 1341 | 2318 | cell division cycle 73, Paf1/RNA polymerase II | 3.662 | 426333-426720 |
| | | complex component, homolog (S. cerevisiae) | | |
| 1353 | 2311 | centrosomal protein 63 | 8.32 | 430642-430998 |
| 1354 | 2311 | high mobility group box 1 | 4.567 | 430999-431370 |
| 1369 | 2302 | protein phosphatase 1, catalytic subunit, | 113.24 | 436277-436523 |
| | | γ isoform | | |
| 1403 | 2287 | structural maintenance of chromosomes 1A | 13.394 | 447520-447805 |
| 1425 | 2276 | minichromosome maintenance deficient 5, | 20.01 | 455118-455499 |
| | | cell division cycle 46 (S. cerevisiae) | | |
| 1438 | 2270 | cysteine and glycine-rich protein 2 | 2.431 | 459534-459931 |
| | | binding protein | | |
| 1505 | 2243 | growth arrest-specific 2 like 1 | 14.15 | 482255-482606 |
| 1523 | 2239 | TSPY-like 2 | 4.364 | 487980-488352 |
| 1532 | 2236 | CDC 16 cell division cycle 16 homolog | 61.55 | 491125-491521 |
| | | (S. cerevisiae) | | |
| 1537 | 2234 | anaphase promoting complex subunit 2 | 8.972 | 492880-493248 |
| 1542 | 2232 | Jun oncogene | 5.841 | 494469-494742 |
| 1554 | 2228 | SUMO/sentrin specific peptidase 5 | 1.726 | 498550-498878 |
| 1557 | 2227 | annexin A11 | 55.57 | 499580-499921 |
| 1560 | 2227 | SET domain containing (lysine | 16.79 | 500465-500805 |
| | | methyltransferase) 8 | | |
| 1562 | 2226 | small G protein signaling modulator 3 | 9.371 | 501162-501548 |
| 1565 | 2224 | ZW10 homolog (Drosophila), centromere/ | 12.63 | 502292-502621 |
| | | kinetochore protein | | |
| 1571 | 2221 | RAD 17 homolog (S. pombe) | 7.172 | 504416-504768 |
| 1582 | 2214 | family with sequence similarity 83, member D | 12.85 | 508106-508316 |
| 1593 | 2210 | rho/rac guanine nucleotide exchange factor (GEF) 2 | 3.451 | 511846-512237 |
| 1608 | 2206 | minichromosome maintenance deficient 3 | 24.19 | 517207-517557 |
| | | (S. cerevisiae) | | |
| 1638 | 2194 | polo-like kinase 2 (Drosophila) | 4.793 | 527681-527996 |
| 1706 | 2173 | catalase | 18.084 | 551058-551444 |
| 1716 | 2169 | cyclin G2 | 4.918 | 554595-554969 |
| 1724 | 2167 | E4F transcription factor 1 | 4.358 | 557314-557678 |
| 1726 | 2166 | cyclin I | 14.85 | 558041-558430 |
| 1741 | 2160 | non-SMC condensin I complex, subunit D2 | 12.081 | 563227-563611 |
| 1743 | 2159 | polymerase (DNA directed) sigma | 11.13 | 563897-564261 |
| 1744 | 2159 | RIKEN cDNA 2400003C14 gene | 16.24 | 564262-564570 |
| 1746 | 2159 | transformation/transcription domain- | 0.661 | 564955-565345 |
| | | associated protein | | |
| 1749 | 2158 | minichromosome maintenance deficient 7 | 52.55 | 566044-566427 |
| | | (S. cerevisiae) | | |
| 1750 | 2158 | retinoblastoma 1 | 1.741 | 566428-566760 |
| 1758 | 2157 | protein phosphatase 1G (formerly 2C), Mg- | 65.51 | 569118-569459 |
| | | dependent, γ isoform | | |
| 1767 | 2154 | programmed cell death 6 interacting protein | 24.67 | 572196-572546 |
| 1822 | 2137 | polo-like kinase 1 (Drosophila) | 42.62 | 591133-591528 |
| 1829 | 2135 | amyloid β (A4) precursor protein-binding, | 13.93 | 593493-593882 |
| | | family B, member 1 | | |
| 1837 | 2132 | polycystic kidney disease 2 | 2.329 | 596164-596507 |
| 1838 | 2132 | proviral integration site 3 | 16.75 | 596508-596892 |
| 1849 | 2128 | NIMA (never in mitosis gene a)-related | 11.135 | 600327-600624 |
| | | expressed kinase 6 | | |
| 1856 | 2126 | SEH1-like (S. cerevisiae) | 6.521 | 602767-603120 |
| 1860 | 2124 | cyclin G1 | 3.56 | 603997-604346 |
| 1874 | 2121 | NIMA (never in mitosis gene a)-related | 5.452 | 608758-609143 |
| | | expressed kinase 9 | | |
| 1882 | 2118 | ubiquitin-like modifier activating enzyme 3 | 26.578 | 611535-611917 |
| 1897 | 2113 | RIKEN cDNA 2010005J08 gene | 3.915 | 616258-616623 |
| 1910 | 2110 | macrophage erythroblast attacher | 48.23 | 620748-621108 |
| 1939 | 2098 | leucine zipper, putative tumor suppressor 2 | 14.19 | 630655-630915 |
| 1944 | 2097 | cell division cycle 42 homolog (S. cerevisiae) | 189.61 | 632324-632630 |
| 1972 | 2086 | protein phosphatase 1, catalytic subunit, | 1.708 | 642111-642462 |
| | | β isoform | | |
| 2029 | 2068 | heat shock protein 8 | 891.02 | 660889-661277 |
| 2078 | 2050 | cyclin F | 3.468 | 677909-678208 |
| 2094 | 2045 | polo-like kinase 3 (Drosophila) | 7.762 | 683175-683550 |
| 2105 | 2042 | CD2-associated protein | 0.744 | 686855-687170 |
| 2111 | 2040 | cyclin D binding myb-like transcription | 1.893 | 688585-688896 |
| | | factor 1 | | |
| 2121 | 2035 | Fanconi anemia, complementation group D2 | 1.038 | 691993-692390 |
| 2131 | 2032 | minichromosome maintenance deficient 2 | 14.00 | 695280-695591 |
| | | mitotin (S. cerevisiae) | | |
| 2139 | 2030 | multiple endocrine neoplasia 1 | 2.911 | 698091-698430 |
| 2182 | 2017 | inhibitor of growth family, member 1 | 6.197 | 712451-712798 |
| 2235 | 2001 | septin 7 | 3.112 | 730587-730976 |
| 2257 | 1993 | cell division cycle 27 homolog (S. cerevisiae) | 0.583 | 738313-738671 |
| 2283 | 1987 | MAP-kinase activating death domain | 1.589 | 747015-747324 |
| 2293 | 1985 | adaptor protein, phosphotyrosine interaction, | 0.781 | 750597-750920 |
| | | PH domain and leucine zipper containing 1 | | |
| 2297 | 1984 | protein phosphatase 3, catalytic subunit, isoform | 4.715 | 751950-752267 |
| 2346 | 1973 | calmodulin 3 | 14.01 | 768392-768693 |
| 2378 | 1963 | ubiquitin-like, containing PHD & RING | 7.038 | 778921-779204 |
| | | finger domains 2 | | |
| 2379 | 1963 | protein regulator of cytokinesis 1 | 14.63 | 779205-779513 |
| 2381 | 1963 | retinoblastoma binding protein 8 | 4.133 | 779852-780237 |
| 2416 | 1954 | kinesin family member C1 | 16.34 | 792040-792370 |
| 2426 | 1951 | adaptor protein, phosphotyrosine interaction, | 2.172 | 795330-795651 |
| | | PH domain and leucine zipper containing 2 | | |
| 2430 | 1949 | anillin, actin binding protein | 2.848 | 796726-797054 |
| 2441 | 1946 | CLIP associating protein 2 | 1.013 | 800461-800731 |
| 2455 | 1943 | host cell factor C1 | 2.096 | 805085-805458 |
| 2471 | 1940 | mutS homolog 2 (E. coli) | 6.134 | 810424-810813 |
| 2474 | 1940 | epiregulin | 9.501 | 811533-811821 |
| 2505 | 1931 | septin 8 | 0.895 | 822293-822664 |
| 2513 | 1930 | DnaJ (Hsp40) homolog, subfamily C, | 34.4 | 825067-825402 |
| | | member 2 | | |
| 2515 | 1929 | Cbp/p300-interacting transactivator, with | 22.655 | 825796-826120 |
| | | Glu/Asp-rich carboxy-terminal domain, 2 | | |
| 2531 | 1925 | NDC80 homolog, kinetochore complex | 20.308 | 831233-831608 |
| | | component (S. cerevisiae) | | |
| 2534 | 1925 | signal-induced proliferation associated gene 1 | 3.696 | 832257-832632 |
| 2547 | 1921 | cell division cycle and apoptosis regulator 1 | 1.757 | 836705-837044 |
| 2562 | 1916 | septin 5 | 22.256 | 841871-842174 |
| 2569 | 1914 | cyclin-dependent kinase 7 (homolog of | 1.788 | 844194-844512 |
| | | Xenopus MO15 cdk-activating kinase) | | |
| 2582 | 1911 | non-SMC condensin I complex, subunit H | 14.505 | 848672-848987 |
| 2583 | 1910 | inner centromere protein | 4.499 | 848988-849386 |
| 2586 | 1910 | par-3 partitioning defective 3 homolog B | 0.422 | 850130-850455 |
| | | (C. elegans) | | |
| 2593 | 1909 | BTG3 associated nuclear protein | 4.134 | 852510-852846 |
| 2595 | 1909 | DBF4 homolog (S. cerevisiae) | 8.657 | 853157-853542 |
| 2608 | 1906 | E2F transcription factor 1 | 7.007 | 857154-857487 |
| 2621 | 1902 | Rac GTPase-activating protein 1 | 19.316 | 861408-861766 |
| 2634 | 1899 | ubiquitin specific peptidase 22 | 1.692 | 865729-866104 |
| 2644 | 1897 | protein phosphatase 2 (formerly 2A), | 46.955 | 869071-869380 |
| | | catalytic subunit, isoform | | |
| 2691 | 1887 | growth arrest specific 2 | 2.282 | 885284-885579 |
| 2693 | 1886 | ring finger protein 2 | 1.202 | 885899-886287 |
| 2707 | 1882 | fizzy/cell division cycle 20 related 1 | 24.719 | 890466-890779 |
| | | (Drosophila) | | |
| 2728 | 1875 | STE20-related kinase adaptor | 12.387 | 897852-898184 |
| 2745 | 1872 | mitotic arrest deficient 1-like 1 | 4.132 | 903571-903958 |
| 2781 | 1861 | histone deacetylase 3 | 24.855 | 916015-916347 |
| 2792 | 1859 | Mdm2, transformed 3T3 cell double minute | 1.49 | 919781-920087 |
| | | p53 binding protein | | |
| 2793 | 1858 | non-SMC condensin II complex, subunit G2 | 2.181 | 920088-920444 |
| 2809 | 1855 | cell division cycle 25 homolog A (S. pombe) | 1.851 | 925695-926050 |
| 2817 | 1854 | regulator of chromosome condensation | 4.485 | 928459-928777 |
| | | (RCC1) and BTB (POZ) domain containing | | |
| | | protein 1 | | |
| 2834 | 1851 | neuroblastoma ras oncogene | 2.46 | 934198-934494 |
| 2844 | 1847 | large tumor suppressor | 0.394 | 937654-937969 |
| 2848 | 1847 | RAD9 homolog (S. pombe) | 13.395 | 938950-939251 |
| 2896 | 1832 | centromere protein E | 1.871 | 955437-955745 |
| 2904 | 1829 | breast cancer 1 | 7.497 | 958124-958436 |
| 2910 | 1827 | cyclin D2 | 1.579 | 960077-960401 |
| 2925 | 1823 | cell division cycle 45 homolog | 5.32 | 965312-965711 |
| | | (S. cerevisiae-like) | | |
| 2968 | 1810 | E2F transcription factor 6 | 4.213 | 980320-980709 |
| 2971 | 1808 | E2F transcription factor 4 | 11.352 | 981429-981759 |
| 2984 | 1804 | Jun-B oncogene | 63.645 | 985798-986175 |
| 3006 | 1794 | retinoblastoma binding protein 4 | 5.65 | 993294-993657 |
| 3033 | 1784 | 3-phosphoglycerate dehydrogenase | 126.19 | 1002179-1002496 |
| 3034 | 1784 | cell division cycle 20 homolog (S. cerevisiae) | 79.792 | 1002497-1002849 |
| 3039 | 1783 | vacuolar protein sorting 4b (yeast) | 2.342 | 1004233-1004573 |
| 3051 | 1779 | suppressor of variegation 3-9 homolog 1 | 2.513 | 1008311-1008610 |
| | | (Drosophila) | | |
| 3066 | 1776 | mitogen-activated protein kinase 3 | 37.586 | 1013377-1013718 |
| 3067 | 1775 | fibroblast growth factor 7 | 1.663 | 1013719-1014044 |
| 3081 | 1772 | septin 6 | 16.844 | 1018327-1018620 |
| 3110 | 1766 | protein kinase, membrane associated tyrosine/ | 10.224 | 1028441-1028755 |
| | | threonine 1 | | |
| 3145 | 1758 | cyclin D 3 | 23.86 | 1040554-1040910 |
| 3149 | 1757 | retinoblastoma-like 2 | 1.946 | 1041915-1042243 |
| 3152 | 1756 | lin-9 homolog (C. elegans) | 0.83 | 1042878-1043200 |
| 3161 | 1755 | E2F transcription factor 8 | 1.759 | 1046151-1046504 |
| 3171 | 1752 | chromatin assembly factor 1, subunit B (p60) | 14.978 | 1049710-1050012 |
| 3177 | 1750 | CDC23 (cell division cycle 23, yeast | 2.323 | 1051775-1052083 |
| | | homolog) | | |
| 3214 | 1742 | RAD50 interactor 1 | 2.415 | 1064421-1064789 |
| 3215 | 1742 | c-abl oncogene 1, receptor tyrosine kinase | 0.436 | 1064790-1065134 |
| 3238 | 1738 | high mobility group AT-hook 2 | 0.823 | 1072519-1072837 |
| 3256 | 1733 | potassium channel tetramerisation domain | 2.201 | 1078388-1078757 |
| | | containing 11 | | |
| 3283 | 1723 | protein phosphatase 1D magnesium- | 2.77 | 1087491-1087869 |
| | | dependent, δ isoform | | |
| 3289 | 1721 | menage a trois 1 | 12.96 | 1089606-1089959 |
| 3301 | 1718 | peripheral myelin protein 22 | 9.401 | 1093771-1094161 |
| 3306 | 1717 | CLIP associating protein 1 | 0.948 | 1095379-1095748 |
| 3338 | 1709 | NEDD8 activating enzyme E1 subunit 1 | 9.826 | 1106097-1106429 |
| 3390 | 1696 | cell division cycle 2-like 1 | 17.014 | 1124002-1124331 |
| 3419 | 1688 | bladder cancer associated protein homolog | 4.537 | 1133723-1134082 |
| | | (human) | | |
| 3426 | 1687 | regulator of chromosome condensation 1 | 4.314 | 1136021-1136304 |
| 3474 | 1673 | cyclin A2 | 5.366 | 1151948-1152332 |
| 3505 | 1668 | katanin p60 (ATPase-containing) subunit A1 | 32.182 | 1162218-1162611 |
| 3551 | 1656 | RIKEN cDNA B230120H23 gene | 0.667 | 1177903-1178190 |
| 3559 | 1654 | SKI-like | 1.243 | 1180446-1180768 |
| 3574 | 1650 | cell division cycle 6 homolog (S. cerevisiae) | 2.478 | 1185367-1185759 |
| 3577 | 1650 | cell division cycle 25 homolog B (S. pombe) | 1.866 | 1186395-1186715 |
| 3583 | 1649 | checkpoint kinase 1 homolog (S. pombe) | 3.146 | 1188354-1188736 |
| 3598 | 1645 | cyclin-dependent kinase 2 | 16.205 | 1193336-1193684 |
| 3604 | 1644 | excision repair cross-complementing rodent | 3.307 | 1195379-1195725 |
| | | repair deficiency complementation group 6 - like | | |
| 3605 | 1644 | vacuolar protein sorting 24 (yeast) | 5.661 | 1195726-1196052 |
| 3652 | 1633 | minichromosome maintenance deficient 8 | 2.747 | 1211842-1212151 |
| | | (S. cerevisiae) | | |
| 3699 | 1623 | transforming, acidic coiled-coil containing | 13.073 | 1227651-1228044 |
| | | protein 3 | | |
| 3705 | 1622 | seven in absentia 2 | 1.664 | 1229814-1230210 |
| 3727 | 1618 | vascular endothelial growth factor C | 10.284 | 1237351-1237686 |
| 3736 | 1616 | cullin 7 | 1.583 | 1240268-1240610 |
| 3743 | 1614 | thioredoxin interacting protein | 5.1 | 1242664-1242964 |
| 3761 | 1609 | ataxia telangiectasia mutated homolog | 0.181 | 1248864-1249255 |
| | | (human) | | |
| 3768 | 1607 | protein (peptidyl-prolyl cis/trans isomerase) | 5.639 | 1251267-1251627 |
| | | NIMA-interacting 1 | | |
| 3773 | 1605 | inhibitor of growth family, member 4 | 12.81 | 1252896-1253239 |
| 3787 | 1601 | transcription factor Dp 1 | 6.434 | 1257788-1258139 |
| 3792 | 1600 | salt inducible kinase 1 | 0.413 | 1259549-1259840 |
| 3804 | 1596 | RIKEN cDNA 6720463M24 gene | 2.973 | 1263541-1263924 |
| 3828 | 1591 | cyclin K | 1.622 | 1271584-1271845 |
| 3855 | 1584 | activating transcription factor 5 | 9.537 | 1280625-1280989 |
| 3865 | 1582 | nuclear autoantigenic sperm protein | 31.057 | 1283868-1284213 |
| | | (histone-binding) | | |
| 3885 | 1577 | SWI/SNF related, matrix associated, actin | 11.687 | 1290692-1291012 |
| | | dependent regulator of chromatin, subfamily b, | | |
| | | member 1 | | |
| 3907 | 1573 | Zwilch, kinetochore associated, homolog | 1.026 | 1297895-1298179 |
| | | (Drosophila) | | |
| 3910 | 1572 | cyclin B1 | 25.641 | 1298863-1299236 |
| 3913 | 1571 | signal transducer & activator of transcription 5 A | 1.268 | 1299843-1300222 |
| 3921 | 1568 | zinc finger protein 369 | 5.039 | 1302401-1302734 |
| 3969 | 1558 | chromatin modifying protein 1A | 7.377 | 1318357-1318651 |
| 4008 | 1547 | Fanconi anemia, complementation group I | 1.721 | 1331437-1331720 |
| 4010 | 1547 | septin 9 | 27.144 | 1332075-1332392 |
| 4016 | 1545 | aryl-hydrocarbon receptor | 0.43 | 1334066-1334367 |
| 4023 | 1544 | Wilms' tumour 1-associating protein | 2.862 | 1336382-1336718 |
| 4069 | 1531 | ubiquitin-like, containing PHD & RING | 6.026 | 1351856-1352193 |
| | | finger domains, 1 | | |
| 4071 | 1530 | NIMA-related expressed kinase 2 | 2.858 | 1352509-1352861 |
| 4090 | 1525 | zinc finger, C3HC type 1 | 17.029 | 1358571-1358886 |
| 4097 | 1523 | RuvB-like protein 1 | 55.736 | 1360967-1361271 |
| 4103 | 1522 | HAUS augmin-like complex, subunit 4 | 20.991 | 1362890-1363204 |
| 4140 | 1514 | E2F transcription factor 5 | 2.277 | 1375653-1375938 |
| 4154 | 1511 | transformed mouse 3T3 cell double minute 2 | 3.215 | 1380172-1380483 |
| 4156 | 1511 | EP300 interacting inhibitor of differentiation 1 | 21.285 | 1380867-1381243 |
| 4160 | 1510 | fibronectin type 3 & SPRY domain- | 2.066 | 1382212-1382607 |
| | | containing protein | | |
| 4171 | 1508 | casein kinase 2, prime polypeptide | 16.889 | 1385888-1386249 |
| 4193 | 1502 | mitogen-activated protein kinase 1 | 15.004 | 1393467-1393856 |
| 4199 | 1500 | cytoskeleton associated protein 2 | 1.674 | 1395624-1396011 |
| 4233 | 1494 | protein phosphatase 6, catalytic subunit | 9.673 | 1407109-1407417 |
| 4255 | 1491 | budding uninhibited by benzimidazoles 1 | 2.264 | 1414236-1414628 |
| | | homolog, β (S. cerevisiae) | | |
| 4266 | 1488 | tumor susceptibility gene 101 | 23.4 | 1417992-1418306 |
| 4268 | 1487 | STE20-related kinase adaptor β | 1.082 | 1418669-1418996 |
| 4290 | 1482 | mutL homolog 1 (E. coli) | 5.514 | 1426359-1426686 |
| 4304 | 1480 | KH domain containing, RNA binding, signal | 4.254 | 1431183-1431494 |
| | | transduction associated 1 | | |
| 4339 | 1472 | helicase, lymphoid specific | 0.521 | 1442541-1442877 |
| 4380 | 1463 | pelota homolog (Drosophila) | 13.919 | 1456293-1456635 |
| 4414 | 1456 | cyclin-dependent kinase 5 | 3.895 | 1467595-1467925 |
| 4476 | 1442 | ring finger protein 8 | 3.436 | 1488202-1488477 |
| 4480 | 1441 | cyclin B2 | 64.86 | 1489394-1489722 |
| 4491 | 1439 | ADP-ribosylation factor-like 8A | 11.733 | 1492911-1493304 |
| 4537 | 1428 | dual specificity phosphatase 1 | 8.225 | 1507891-1508212 |
| 4554 | 1425 | growth arrest and DNA-damage-inducible, β | 3.26 | 1513621-1513922 |
| | | interacting protein 1 | | |
| 4632 | 1407 | cell division cycle 7 (S. cerevisiae) | 2.07 | 1539427-1539781 |
| 4685 | 1394 | annexin A1 | 186.99 | 1557035-1557427 |
| 4702 | 1391 | chromatin licensing and DNA replication factor 1 | 5.76 | 1563109-1563436 |
| 4728 | 1387 | acidic (leucine-rich) nuclear phosphoprotein | 140.45 | 1571589-1571985 |
| | | 32 family, member B | | |
| 4729 | 1387 | regulator of chromosome condensation 2 | 9.39 | 1571986-1572324 |
| 4732 | 1386 | sirtuin 2 (silent mating type information | 9.325 | 1573015-1573411 |
| | | regulation 2, homolog) (S. cerevisiae) | | |
| 4747 | 1383 | seven in absentia 1A | 1.166 | 1578078-1578382 |
| 4775 | 1373 | ecotropic viral integration site 5 | 1.536 | 1587335-1587660 |
| 4777 | 1373 | zinc finger protein 830 | 2.475 | 1587986-1588305 |
| 4792 | 1371 | protein phosphatase 1, catalytic subunit, | 294.16 | 1593376-1593702 |
| | | isoform | | |
| 4811 | 1366 | coiled-coil domain containing 99 | 1.214 | 1599899-1600288 |
| 4839 | 1360 | cyclin-dependent kinase 4 | 100.24 | 1609522-1609852 |
| 4882 | 1350 | nuclear factor of activated T-cells, | 0.584 | 1623871-1624266 |
| | | cytoplasmic, calcineurin-dependent 1 | | |
| 4893 | 1348 | vacuolar protein sorting 4a (yeast) | 1.43 | 1627799-1628173 |
| 4897 | 1348 | anaphase promoting complex subunit 7 | 3.347 | 1629252-1629559 |
| 4957 | 1336 | transformation related p53 | 6.608 | 1649857-1650157 |
| 4969 | 1332 | TGFβ-regulated gene 1 | 16.316 | 1654114-1654473 |
| 4976 | 1330 | nucleoporin 214 | 0.854 | 1656631-1657026 |
| 4978 | 1330 | homeo box B4 | 1.659 | 1657427-1657747 |
| 5039 | 1316 | S-phase kinase-associated protein 2 (p45) | 0.814 | 1678054-1678363 |
| 5104 | 1300 | nuclear distribution gene C homolog | 102.46 | 1699971-1700369 |
| | | (Aspergillus) | | |
| 5201 | 1281 | cyclin D-type binding-protein 1 | 14.126 | 1733399-1733721 |
| 5208 | 1279 | nucleolar and spindle associated protein 1 | 2.386 | 1735724-1736042 |
| 5221 | 1275 | growth arrest and DNA-damage-inducible 45 β | 21.495 | 1740423-1740753 |
| 5268 | 1267 | F-box protein 5 | 1.752 | 1756030-1756337 |
| 5277 | 1265 | COP9 (constitutive photomorphogenic) | 22.12 | 1759189-1759545 |
| | | homolog, subunit 5 (Arabidopsis) | | |
| 5287 | 1263 | nucleophosmin 1 | 155.72 | 1762731-1763125 |
| 5319 | 1255 | chromatin modifying protein 1B | 4.81 | 1773630-1773932 |
| 5357 | 1245 | TGFβ1 | 13.689 | 1787146-1787456 |
| 5370 | 1243 | HAUS augmin-like complex, subunit 7 | 59.234 | 1791926-1792280 |
| 5373 | 1242 | H2A histone family, member X | 35.377 | 1792920-1793310 |
| 5389 | 1239 | high mobility group 20 B | 18.123 | 1798703-1799083 |
| 5399 | 1238 | RAN, member RAS Oncogene family | 61.23 | 1802120-1802418 |
| 5401 | 1237 | nucleoporin 37 | 8.371 | 1802806-1803091 |
| 5443 | 1227 | CHK2 checkpoint homolog (S. pombe) | 1.749 | 1817364-1817648 |
| 5448 | 1226 | RIKEN cDNA F630043A04 gene | 2.085 | 1819143-1819511 |
| 5459 | 1223 | BRCA2 and CDKN1A interacting protein | 22.32 | 1823218-1823604 |
| 5476 | 1218 | cell division cycle 123 homolog | 19.04 | 1829272-1829545 |
| | | (S. cerevisiae) | | |
| 5513 | 1209 | NIMA-related expressed kinase 1 | 0.751 | 1842362-1842733 |
| 5531 | 1204 | DNA cross-link repair 1A, PSO2 homolog | 0.722 | 1848560-1848902 |
| | | (S. cerevisiae) | | |
| 5560 | 1198 | forkhead box N3 | 0.714 | 1858644-1859006 |
| 5569 | 1196 | nibrin | 0.874 | 1861722-1862120 |
| 5580 | 1194 | cell division cycle 2 homolog A (S. pombe) | 43.513 | 1865374-1865693 |
| 5609 | 1188 | F-box protein 31 | 1.331 | 1875647-1875991 |
| 5636 | 1182 | mitogen-activated protein kinase 7 | 1.049 | 1885325-1885696 |
| 5653 | 1178 | apoptosis antagonizing transcription factor | 19.78 | 1891250-1891647 |
| 5667 | 1173 | reprimo, TP53 dependent G2 arrest mediator | 2.891 | 1896225-1896560 |
| | | candidate | | |
| 5676 | 1171 | cell growth regulator with ring finger domain 1 | 8.143 | 1899574-1899946 |
| 5694 | 1168 | vascular endothelial growth factor B | 11.40 | 1906017-1906367 |
| 5698 | 1166 | aurora kinase A | 16.86 | 1907469-1907831 |
| 5701 | 1166 | telomeric repeat binding factor 1 | 2.789 | 1908582-1908967 |
| 5729 | 1161 | MAD2 mitotic arrest deficient-like 2 (yeast) | 23.02 | 1918682-1919036 |
| 5746 | 1157 | caspase 3 | 11.813 | 1924836-1925195 |
| 5773 | 1151 | protein tyrosine phosphatase 4a1 | 0.279 | 1934685-1935079 |
| 5774 | 1151 | centrobin, centrosomal BRCA2 interacting | 1.021 | 1935080-1935410 |
| | | protein | | |
| 5787 | 1148 | mitochondrial tumor suppressor 1 | 0.27 | 1939909-1940301 |
| 5828 | 1140 | growth arrest and DNA-damage-inducible 45 | 21.77 | 1954514-1954899 |
| 5833 | 1139 | cyclin H | 10.28 | 1956302-1956671 |
| 5869 | 1132 | cyclin-dependent kinase inhibitor 2C (p18, | 13.76 | 1969649-1970047 |
| | | inhibits CDK4) | | |
| 5877 | 1129 | E2F transcription factor 7 | 0.593 | 1972492-1972861 |
| 5881 | 1129 | mediator of DNA damage checkpoint 1 | 0.237 | 1974024-1974400 |
| 5882 | 1129 | calmodulin 2 | 263.81 | 1974401-1974748 |
| 5899 | 1124 | cyclin E1 | 1.228 | 1980613-1981009 |
| 5902 | 1124 | cell cycle related kinase | 3.686 | 1981792-1982170 |
| 5927 | 1118 | cyclin-dependent kinase inhibitor 2D (p19, | 10.528 | 1990790-1991181 |
| | | inhibits CDK4) | | |
| 5933 | 1117 | thioredoxin-like 4A | 29.973 | 1993063-1993439 |
| 5997 | 1101 | NUF2, NDC80 kinetochore complex | 1.166 | 2016390-2016751 |
| | | component, homolog (S. cerevisiae) | | |
| 6008 | 1100 | DSN1, MIND kinetochore complex | 2.499 | 2020156-2020546 |
| | | component, homolog (S. cerevisiae) | | |
| 6049 | 1092 | RIKEN cDNA 2610002M06 gene | 2.988 | 2035042-2035392 |
| 6060 | 1089 | cell division cycle associated 8 | 7.204 | 2039068-2039461 |
| 6065 | 1088 | asp (abnormal spindle)-like, microcephaly | 0.54 | 2040964-2041345 |
| | | associated (Drosophila) | | |
| 6084 | 1083 | bridging integrator 3 | 4.997 | 2047682-2048036 |
| 6119 | 1075 | ankyrin repeat domain 54 | 3.785 | 2060520-2060872 |
| 6130 | 1072 | proline/serine-rich coiled-coil 1 | 1.861 | 2064611-2064994 |
| 6141 | 1070 | aurora kinase B | 6.311 | 2068620-2068994 |
| 6153 | 1068 | max binding protein | 0.824 | 2073201-2073580 |
| 6173 | 1064 | CDK2 (cyclin-dependent kinase 2)- | 21.227 | 2079920-2080306 |
| | | associated protein 1 | | |
| 6246 | 1047 | CDK5 and Abl enzyme substrate 1 | 0.472 | 2106649-2107036 |
| 6309 | 1031 | CDK5 and Abl enzyme substrate 2 | 3.54 | 2128521-2128907 |
| 6318 | 1030 | centrin 2 | 4.69 | 2131765-2132103 |
| 6434 | 1005 | telomeric repeat binding factor 2 | 1.302 | 2173556-2173867 |
| 6480 | 992 | cyclin-dependent kinase 6 | 1.042 | 2189891-2190242 |
| 6534 | 981 | discs, large (Drosophila) homolog- | 3.759 | 2208846-2209155 |
| | | associated protein 5 | | |
| 6553 | 976 | RIKEN cDNA 2810433K01 gene | 2.289 | 2215581-2215976 |
| 6574 | 973 | checkpoint w/forkhead & ring finger domains | 0.59 | 2222821-2223198 |
| 6581 | 971 | HAUS augmin-like complex, subunit 1 | 5.105 | 2225453-2225779 |
| 6647 | 960 | Bmi1 polycomb ring finger oncogene | 0.42 | 2248765-2249118 |
| 6664 | 956 | par-6 (partitioning defective 6,) homolog | 1.905 | 2254717-2255106 |
| | | (C. elegans) | | |
| 6669 | 955 | ras homolog gene family, member U | 0.296 | 2256530-2256915 |
| 6678 | 952 | BCL2-antagonist/killer 1 | 3.0 | 2259855-2260161 |
| 6713 | 943 | centrosomal protein 250 | 0.433 | 2271720-2272085 |
| 6714 | 942 | centromere protein O | 0.733 | 2272086-2272464 |
| 6729 | 939 | kinesin family member 11 | 1.155 | 2277436-2277733 |
| 6782 | 929 | nuclear distribution gene E homolog 1 | 7.884 | 2295836-2296146 |
| | | (A. nidulans) | | |
| 6812 | 922 | forkhead box O4 | 1.102 | 2306279-2306609 |
| 6827 | 918 | protein kinase inhibitor | 0.376 | 2311372-2311759 |
| 6833 | 917 | septin 3 | 0.248 | 2313405-2313686 |
| 6882 | 908 | aurora kinase C | 14.22 | 2329723-2330035 |
| 6898 | 903 | spindle assembly 6 homolog (C. elegans) | 0.224 | 2334515-2334801 |
| 6909 | 902 | septin 10 | 4.725 | 2337987-2338293 |
| 6952 | 892 | timeless interacting protein | 1.598 | 2352399-2352710 |
| 7003 | 880 | neural precursor cell expressed, | 0.33 | 2369333-2369684 |
| | | developmentally down-regulated gene 1 | | |
| 7010 | 877 | proteasome (prosome, macropain) assembly | 9.024 | 2371767-2372110 |
| | | chaperone 2 | | |
| 7115 | 858 | centromere protein H | 4.014 | 2406674-2407073 |
| 7126 | 852 | vasohibin 1 | 0.138 | 2410493-2410795 |
| 7151 | 847 | germ cell-specific gene 2 | 0.717 | 2418878-2419222 |
| 7158 | 845 | c-fos induced growth factor | 3.445 | 2420809-2421101 |
| 7159 | 845 | MAD2 mitotic arrest deficient-like 1 (yeast) | 8.32 | 2421102-2421467 |
| 7175 | 841 | baculoviral IAP repeat-containing 5 | 0.966 | 2426437-2426713 |
| 7199 | 838 | TGFβ3 | 1.124 | 2434410-2434754 |
| 7208 | 836 | Leu rich repeat & coiled-coil domain containing 1 | 0.248 | 2437517-2437910 |
| 7216 | 834 | suppressor of variegation 3-9 homolog 2 | 0.735 | 2440171-2440490 |
| | | (Drosophila) | | |
| 7224 | 833 | NIMA (never in mitosis gene a)-related | 0.325 | 2443004-2443301 |
| | | expressed kinase 4 | | |
| 7228 | 832 | cell division cycle 25 homolog C (S. pombe) | 1.85 | 2444341-2444625 |
| 7249 | 826 | RIKEN cDNA 4922501C03 gene | 0.438 | 2451461-2451761 |
| 7283 | 816 | ribosomal protein S6 | 18.875 | 2462245-2462567 |
| 7291 | 813 | HAUS augmin-like complex, subunit 2 | 3.496 | 2464662-2464966 |
| 7330 | 803 | MAD2L1 binding protein | 3.685 | 2477746-2478077 |
| 7365 | 796 | cDNA sequence BC023882 | 0.603 | 2489301-2489640 |
| 7405 | 788 | cell division cycle associated 3 | 10.276 | 2502489-2502808 |
| 7439 | 778 | B-cell leukemia/lymphoma 2 | 0.149 | 2513854-2514170 |
| 7444 | 776 | cell division cycle associated 2 | 0.252 | 2515580-2515941 |
| 7454 | 774 | platelet derived growth factor, | 4.859 | 2518844-2519200 |
| 7551 | 749 | expressed sequence C79407 | 0.187 | 2550344-2550743 |
| 7554 | 749 | enhancer of rudimentary homolog | 3.142 | 2551508-2551815 |
| | | (Drosophila) | | |
| 7565 | 747 | CDC28 protein kinase 1b | 22.475 | 2555228-2555394 |
| 7603 | 738 | SPC24, NDC80 kinetochore complex | 1.038 | 2567431-2567712 |
| | | component, homolog (S. cerevisiae) | | |
| 7630 | 732 | serine/threonine kinase 11 | 0.449 | 2575716-2576017 |
| 7633 | 731 | anaphase promoting complex subunit 10 | 0.787 | 2576622-2576942 |
| 7674 | 720 | malignant T cell amplified sequence 1 | 1.817 | 2590027-2590422 |
| 7720 | 710 | arginine vasopressin-induced 1 | 19.275 | 2605845-2606126 |
| 7756 | 700 | Rap1 interacting factor 1 homolog (yeast) | 0.083 | 2618117-2618471 |
| 7781 | 693 | proviral integration site 1 | 0.392 | 2626615-2627001 |
| 7795 | 691 | pituitary tumor-transforming gene 1 | 3.612 | 2631430-2631804 |
| 7803 | 689 | breast cancer 2 | 0.07 | 2634236-2634594 |
| 7838 | 679 | par-6 (partitioning defective 6) homolog β | 0.235 | 2645826-2646140 |
| | | (C. elegans) | | |
| 7841 | 678 | NIMA (never in mitosis gene a)-related | 0.716 | 2646895-2647246 |
| | | expressed kinase 3 | | |
| 7864 | 669 | amyloid beta (A4) precursor protein-binding, | 0.212 | 2654750-2655139 |
| | | family B, member 2 | | |
| 7879 | 666 | cyclin-dependent kinase inhibitor 1A (P21) | 3.252 | 2659502-2659871 |
| 7888 | 664 | StAR-related lipid transfer (START) domain | 0.12 | 2662630-2662978 |
| | | containing 13 | | |
| 7899 | 659 | ADP-ribosylation factor-like 3 | 2.999 | 2666529-2666853 |
| 7957 | 644 | RIKEN cDNA 2810452K22 gene | 4.522 | 2686201-2686541 |
| 8038 | 618 | polyamine-modulated factor 1 | 5.098 | 2712675-2712999 |
| 8046 | 615 | cell division cycle associated 5 | 1.215 | 2715194-2715557 |
| 8079 | 606 | ADP-ribosylation factor-like 2 | 4.584 | 2726337-2726723 |
| 8095 | 601 | cyclin-dependent kinase inhibitor 1B | 0.381 | 2731076-2731440 |
| 8100 | 601 | E2F transcription factor 2 | 0.204 | 2732428-2732782 |
| 8123 | 594 | citron | 0.131 | 2740025-2740319 |
| 8155 | 587 | sphingomyelin phosphodiesterase 3, neutral | 0.179 | 2750331-2750645 |
| 8174 | 583 | mitochondrial ribosomal protein L41 | 0.749 | 2755819-2756155 |
| 8176 | 583 | dynactin 3 | 1.37 | 2756466-2756744 |
| 8209 | 573 | CDC28 protein kinase regulatory subunit 2 | 0.994 | 2767357-2767753 |
| 8220 | 569 | geminin | 1.653 | 2770484-2770876 |
| 8281 | 552 | ubiquitin-conjugating enzyme E2C | 2.402 | 2790466-2790755 |
| 8283 | 551 | SPC25, NDC80 kinetochore complex | 4.035 | 2791059-2791454 |
| | | component, homolog (S. cerevisiae) | | |
| 8300 | 548 | MIS12 homolog (yeast) | 0.199 | 2796988-2797361 |
| 8308 | 544 | NSL1, MIND kinetochore complex | 0.644 | 2799438-2799722 |
| | | component, homolog (S. cerevisiae) | | |
| 8335 | 539 | par-3 (partitioning defective 3) homolog | 0.154 | 2808716-2809107 |
| | | (C. elegans) | | |
| 8348 | 536 | myeloid leukemia factor 1 | 0.454 | 2813229-2813621 |
| 8349 | 535 | DNA-damage inducible transcript 3 | 4.982 | 2813622-2813956 |
| 8378 | 526 | RIKEN cDNA 2610039C10 gene | 2.336 | 2823614-2823897 |
| 8390 | 522 | RAD50 homolog (S. cerevisiae) | 0.102 | 2827676-2828022 |
| 8393 | 522 | proline rich 5 (renal) | 0.462 | 2828643-2828993 |
| 8398 | 520 | cell division cycle 26 | 6.939 | 2830505-2830878 |
| 8402 | 519 | ciliary rootlet coiled-coil, rootletin | 0.102 | 2831925-2832268 |
| 8429 | 512 | ligase IV, DNA, ATP-dependent | 0.41 | 2841502-2841815 |
| 8594 | 478 | cyclin-dependent kinase inhibitor 2B (p15, | 2.07 | 2895015-2895359 |
| | | inhibits CDK4) | | |
| 8620 | 473 | cyclin E2 | 0.159 | 2904183-2904530 |
| 8643 | 468 | RIKEN cDNA 9130404D08 gene | 0.284 | 2912093-2912444 |
| 8680 | 462 | 4HAUS augmin-like complex, subunit 8 | 1.099 | 2923769-2924049 |
| 8765 | 447 | tet oncogene family member 2 | 0.115 | 2950714-2950987 |
| 8776 | 445 | TAF10 RNA polymerase II, TATA box | 1.068 | 2953967-2954306 |
| | | binding protein (TBP)-associated factor | | |
| 8808 | 439 | centrin 3 | 1.05 | 2963461-2963764 |
| 8817 | 437 | S100 calcium binding protein A6 (calcyclin) | 10.781 | 2966362-2966657 |
| 8877 | 422 | thioredoxin-like 4B | 0.561 | 2984204-2984528 |
| 8905 | 416 | K(lysine) acetyltransferase 2B | 0.097 | 2992304-2992627 |
| 8932 | 409 | SAC3 domain containing 1 | 0.452 | 2999825-3000109 |
| 8933 | 409 | ZW10 interactor | 0.203 | 3000110-3000428 |
| 8954 | 403 | junction-mediating and regulatory protein | 0.09 | 3005715-3006035 |
| 8964 | 401 | establishment of cohesion 1 homolog 2 | 0.202 | 3008545-3008860 |
| | | (S. cerevisiae) | | |
| 9019 | 388 | BCL2-associated X protein | 1.131 | 3023234-3023515 |
| 9107 | 363 | growth arrest-specific 2 like 3 | 0.094 | 3045621-3045858 |
| 9152 | 352 | anaphase promoting complex subunit 13 | 0.808 | 3056640-3056878 |
| 9161 | 349 | RAB GTPase activating protein 1 | 0.083 | 3058415-3058689 |
| 9360 | 297 | structural maintenance of chromosomes 4 | 0.073 | 3101933-3102112 |
| 9392 | 286 | cyclin T1 | 0.131 | 3107706-3107919 |
| 9409 | 280 | anaphase promoting complex subunit 11 | 2.158 | 3111178-3111374 |
| 9434 | 270 | growth arrest specific 1 | 0.093 | 3115802-3115978 |
| 9477 | 255 | shugoshin-like 1 (S. pombe) | 0.109 | 3123096-3123285 |
| 9504 | 241 | protein tyrosine phosphatase, receptor type, V | 0.04 | 3127383-3127553 |
| 9512 | 239 | G protein-coupled receptor 132 | 0.098 | 3128448-3128607 |
| 9665 | 179 | regulator of G-protein signaling 2 | 0.059 | 3145905-3146047 |
| 9740 | 139 | betacellulin, EGF family member | 0.073 | 3150839-3150877 |
| 3157247 | 594 | endoplasmic reticulum to nucleus signaling 1 | 0.18 | 3179284-3179383 |
| 3157294 | 353 | family with sequence similarity 33, member A | 0.31 | 3266905-3267004 |
| 3157319 | 402 | HAUS augmin-like complex, subunit 5 | 0.183 | 3232617-3232716 |
| 3157349 | 680 | NA | 7.561 | 3271596-3271695 |
| 3157464 | 268 | TMF1-regulated nuclear protein 1 | 0.324 | 3280149-3280248 |
| 3157487 | 308 | NIMA (never in mitosis gene a)-related | 0.493 | 3167184-3167283 |
| | | expressed kinase 11 | | |
| 3157523 | 803 | centromere protein V | 3.696 | 3267205-3267304 |
| 3157530 | 446 | adenylate kinase 1 | 0.22 | 3212658-3212757 |
| 3157631 | 3098 | establishment of cohesion 1 homolog 1 | 4.952 | 3198571-3198670 |
| | | (S. cerevisiae) | | |
| 3157646 | 403 | hepatic nuclear factor 4, | 0.092 | 3262105-3262204 |
| 3157712 | 3480 | structural maintenance of chromosomes 2 | 1.498 | 3189471-3189570 |
| 3157780 | 1357 | PEST proteolytic signal containing | 2.21 | 3191171-3191270 |
| | | nuclear protein | | |
| 3157798 | 765 | speedy homolog A (Xenopus) | 0.756 | 3226217-3226316 |
| 3157809 | 1876 | NA | 0.817 | 3262505-3262604 |
| 3157812 | 168 | sestrin 2 | 0.071 | 3260905-3261004 |
| 3157837 | 1573 | caspase 8 associated protein 2 | 0.5 | 3184971-3185070 |
| 3157862 | 2352 | retinoblastoma-like 1 (p107) | 2.545 | 3265505-3265604 |
| 3157928 | 393 | NA | 0.096 | 3213958-3214057 |
| 3157931 | 840 | podoplanin | 8.076 | 3202997-3203096 |
| 3157962 | 4088 | NA | 15.347 | 3158921-3159020 |
| 3157993 | 162 | epidermal growth factor receptor | 0.048 | 3166784-3166883 |
| 3158035 | 383 | septin 1 | 0.279 | 3259205-3259304 |
| 3158037 | 432 | phospholipase A2, group XVI | 0.124 | 3230917-3231016 |
| 3158121 | 3735 | p53-inducible nuclear protein 1 | 2.567 | 3197071-3197170 |
| 3158132 | 612 | RIKEN cDNA 4632434I11 gene | 0.26 | 3275096-3275195 |
| 3158184 | 776 | calcium/calmodulin-dependent protein kinase II | 0.183 | 3257905-3258004 |
| 3158209 | 1954 | fibroblast growth factor receptor 2 | 2.109 | 3207458-3207557 |
| 3158213 | 203 | deleted in bladder cancer 1 (human) | 0.113 | 3194971-3195070 |
| 3158218 | 4830 | NA | 2.47 | 3259005-3259104 |
| 3158295 | 426 | stratifin | 0.681 | 3216091-3216190 |
| 3158307 | 369 | placental growth factor | 0.923 | 3227817-3227916 |
| 3158328 | 1531 | RAB11 family interacting protein 3 (class II) | 0.975 | 3221991-3222090 |

TABLE 14
Apoptosis (Chinese hamster)

| SEQ | | | Avg | siRNA |
|---|---|---|---|---|
| ID NO: | consL | Description | Cov | SEQ ID NOs: |
| 16 | 4536 | homeodomain interacting protein kinase 1 | 5.166 | 14439-14801 |
| 21 | 4379 | feminization 1 homolog b (C. elegans) | 5.83 | 15971-16283 |
| 31 | 4290 | nuclear receptor subfamily 3, | 6.926 | 19057-19428 |
| | | group C, member 1 | | |
| 44 | 4201 | SH3-domain kinase binding protein 1 | 6.615 | 23443-23756 |
| 73 | 3972 | cell adhesion molecule 1 | 13.147 | 32944-33332 |
| 102 | 3754 | neurofibromatosis 1 | 1.523 | 42422-42742 |
| 104 | 3746 | PHD finger protein 17 | 2.772 | 43019-43313 |
| 111 | 3699 | intersectin 1 (SH3 domain protein 1A) | 3.481 | 45218-45546 |
| 131 | 3611 | mitogen-activated protein kinase 9 | 5.629 | 51635-51907 |
| 170 | 3445 | RING1 and YY1 binding protein | 15.89 | 63269-63644 |
| 189 | 3384 | phosphatase and tensin homolog | 0.633 | 69091-69404 |
| 199 | 3345 | protein kinase C, | 2.25 | 72112-72439 |
| 204 | 3339 | sphingosine phosphate lyase 1 | 2.842 | 73601-73949 |
| 205 | 3337 | unc-5 homolog B (C. elegans) | 15.951 | 73950-74213 |
| 218 | 3290 | alanyl-tRNA synthetase | 25.07 | 77662-77970 |
| 243 | 3224 | Fas-associated factor 1 | 10.626 | 85018-85295 |
| 266 | 3179 | vascular endothelial growth factor A | 18.713 | 92246-92594 |
| 272 | 3152 | Rho-associated coiled-coil containing | 3.17 | 94052-94292 |
| | | protein kinase 1 | | |
| 279 | 3139 | methyl CpG binding protein 2 | 1.23 | 95910-96141 |
| 293 | 3127 | SAFB-like, transcription modulator | 10.672 | 100152-100477 |
| 300 | 3115 | nischarin | 3.465 | 102105-102309 |
| 345 | 3034 | glycogen synthase kinase 3 β | 0.647 | 114424-114743 |
| 366 | 3003 | cullin 1 | 25.78 | 120499-120798 |
| 375 | 2989 | RAD21 homolog (S. pombe) | 34.322 | 123260-123508 |
| 384 | 2977 | transcriptional regulator, SIN3A (yeast) | 3.56 | 125791-126119 |
| 386 | 2976 | cytotoxic granule-associated RNA binding | 1.496 | 126356-126593 |
| | | protein 1 | | |
| 390 | 2967 | tumor necrosis factor receptor | 22.566 | 127481-127779 |
| | | superfamily, member 21 | | |
| 394 | 2960 | apoptosis inhibitor 5 | 2.055 | 128748-129043 |
| 419 | 2931 | dedicator of cytokinesis 1 | 4.621 | 135539-135925 |
| 431 | 2919 | Tia1 cytotoxic granule-associated RNA | 12.569 | 139041-139241 |
| | | binding protein-like 1 | | |
| 434 | 2913 | mitogen-activated protein kinase kinase | 2.174 | 139905-140195 |
| | | kinase 7 | | |
| 511 | 2835 | hypoxia inducible factor 1, subunit | 6.799 | 161268-161478 |
| 525 | 2821 | BCL2-like 13 (apoptosis facilitator) | 7.089 | 165351-165590 |
| 540 | 2799 | signal transducer and activator of | 1.323 | 169415-169753 |
| | | transcription 5B | | |
| 543 | 2791 | Janus kinase 2 | 4.149 | 170408-170768 |
| 562 | 2773 | uveal autoantigen with coiled-coil | 14.96 | 175535-175851 |
| | | domains and ankyrin repeats | | |
| 609 | 2735 | activity-dependent neuroprotective protein | 6.52 | 189603-189839 |
| 621 | 2718 | catenin | 30.996 | 192742-193116 |
| 670 | 2686 | nuclear factor of kappa light polypeptide | 4.948 | 208013-208351 |
| | | gene enhancer in B-cells 1, p105 | | |
| 710 | 2658 | TNF receptor-associated factor 3 | 1.284 | 219648-219935 |
| 729 | 2640 | homeodomain interacting protein kinase 3 | 0.784 | 225660-225908 |
| 732 | 2639 | transforming growth factor, β receptor I | 4.064 | 226652-227037 |
| 825 | 2573 | amyloid β (A4) precursor protein | 165.22 | 255412-255644 |
| 867 | 2552 | sphingomyelin synthase 1 | 9.259 | 268366-268630 |
| 891 | 2542 | RB1-inducible coiled-coil 1 | 2.069 | 275944-276175 |
| 899 | 2538 | p53-binding protein 2 | 2.893 | 278604-278960 |
| 901 | 2538 | zinc finger matrin type 3 | 0.701 | 279250-279506 |
| 913 | 2532 | proteaseome (prosome, macropain) 28 | 21.397 | 283197-283568 |
| | | subunit, 3 | | |
| 933 | 2523 | phosphatidylinositol 3-kinase, catalytic, | 1.26 | 290027-290396 |
| | | polypeptide | | |
| 994 | 2485 | Tax1 (human T-cell leukemia virus type I) | 26.472 | 310231-310562 |
| | | binding protein 1 | | |
| 1001 | 2480 | myeloid cell leukemia sequence 1 | 11.498 | 312684-312913 |
| 1046 | 2453 | TNF receptor-associated factor 7 | 17.763 | 327682-328074 |
| 1070 | 2440 | promyelocytic leukemia | 1.141 | 335490-335874 |
| 1096 | 2428 | synovial apoptosis inhibitor 1, synoviolin | 3.957 | 344178-344523 |
| 1116 | 2418 | mutS homolog 6 (E. coli) | 11.162 | 350996-351268 |
| 1121 | 2417 | ubiquitin-conjugating enzyme | 3.951 | 352601-352956 |
| | | E2Z (putative) | | |
| 1230 | 2367 | mitogen-activated protein kinase 8 | 0.908 | 388975-389185 |
| 1237 | 2364 | rabaptin, RAB GTPase binding effector | 1.86 | 391313-391594 |
| | | protein 1 | | |
| 1285 | 2341 | D4, zinc and double PHD fingers family 2 | 14.055 | 407477-407781 |
| 1286 | 2340 | RNA binding motif protein 5 | 6.953 | 407782-408116 |
| 1329 | 2323 | adenomatosis polyposis coli | 0.997 | 422123-422508 |
| 1340 | 2318 | GRAM domain containing 4 | 3.878 | 426012-426332 |
| 1381 | 2294 | vac 14 homolog (S. cerevisiae) | 7.275 | 440226-440553 |
| 1386 | 2293 | serine incorporator 3 | 64.3 | 441950-442265 |
| 1398 | 2290 | phosphatidylinositol 3-kinase, regulatory | 0.933 | 445880-446276 |
| | | subunit, polypeptide 1 (p85) | | |
| 1430 | 2274 | ring finger protein 216 | 2.663 | 456856-457171 |
| 1458 | 2262 | Alstrom syndrome 1 homolog (human) | 0.712 | 466342-466731 |
| 1468 | 2259 | HLA-B-associated transcript 3 | 18.8 | 469774-470120 |
| 1469 | 2258 | RIKEN cDNA 5730403B10 gene | 2.351 | 470121-470460 |
| 1491 | 2250 | BCL2-like 2 | 6.539 | 477629-477999 |
| 1520 | 2239 | optic atrophy 1 homolog (human) | 2.52 | 487010-487405 |
| 1547 | 2230 | mitogen-activated protein kinase 8 | 5.814 | 496124-496454 |
| | | interacting protein 1 | | |
| 1561 | 2227 | autophagy/beclin 1 regulator 1 | 1.709 | 500806-501161 |
| 1572 | 2221 | glutaminyl-tRNA synthetase | 17.276 | 504769-505049 |
| 1596 | 2209 | Kruppel-like factor 11 | 2.24 | 512866-513206 |
| 1610 | 2205 | ankyrin 2, brain | 0.639 | 517928-518264 |
| 1615 | 2204 | interleukin-1 receptor-associated kinase 2 | 7.953 | 519606-519900 |
| 1617 | 2203 | BCL2/adenovirus E1B interacting protein | 4.764 | 520293-520639 |
| | | 3-like | | |
| 1623 | 2201 | v-raf-leukemia viral Oncogene 1 | 11.737 | 522454-522805 |
| 1625 | 2201 | carbohydrate sulfotransferase 11 | 1.436 | 523162-523531 |
| 1640 | 2194 | p21 protein (Cdc42/Rac)-activated kinase 2 | 12.908 | 528351-528713 |
| 1670 | 2185 | GATA zinc finger domain containing 2A | 6.186 | 538783-539093 |
| 1681 | 2181 | brain derived neurotrophic factor | 1.421 | 542519-542783 |
| 1745 | 2159 | huntingtin interacting protein 1 | 2.993 | 564571-564954 |
| 1767 | 2154 | programmed cell death 6 | 24.67 | 572196-572546 |
| | | interacting protein | | |
| 1793 | 2146 | thymoma viral proto-oncogene 1 | 55.121 | 581286-581643 |
| 1807 | 2140 | prion protein | 10.293 | 586022-586407 |
| 1814 | 2138 | autophagy-related 7 (yeast) | 3.031 | 588504-588828 |
| 1828 | 2136 | matrix metallopeptidase 9 | 16.33 | 593202-593492 |
| 1829 | 2135 | amyloid beta (A4) precursor protein- | 13.93 | 593493-593882 |
| | | binding, family B, member 1 | | |
| 1849 | 2128 | NIMA (never in mitosis gene a)-related | 11.135 | 600327-600624 |
| | | expressed kinase 6 | | |
| 1866 | 2123 | huntingtin | 0.879 | 606012-606402 |
| 1913 | 2108 | apoptosis-inducing factor, mitochondrion- | 114.54 | 621815-622188 |
| | | associated 1 | | |
| 1925 | 2104 | DnaJ (Hsp40) homolog, subfamily A, | 15.15 | 625909-626254 |
| | | member 3 | | |
| 1943 | 2097 | chromodomain helicase DNA binding | 3.526 | 631928-632323 |
| | | protein 8 | | |
| 1963 | 2088 | tumor necrosis factor receptor | 0.748 | 638890-639228 |
| | | superfamily, member 1b | | |
| 1967 | 2087 | serum/glucocorticoid regulated kinase 1 | 4.001 | 640401-640729 |
| 1971 | 2086 | Scl/Tal1 interrupting locus | 0.813 | 641737-642110 |
| 1974 | 2086 | lymphotoxin B receptor | 20.795 | 642821-643161 |
| 1985 | 2083 | serine/threonine kinase 4 | 2.64 | 646540-646922 |
| 2003 | 2079 | X-ray repair complementing defective | 5.752 | 652584-652920 |
| | | repair in CHO cells 5 | | |
| 2017 | 2073 | myocyte enhancer factor 2D | 8.208 | 657055-657357 |
| 2021 | 2072 | B-cell translocation gene 2, | 5.326 | 658375-658645 |
| | | anti-proliferative | | |
| 2024 | 2071 | K(lysine) acetyltransferase 2A | 4.934 | 659254-659597 |
| 2040 | 2064 | STE20-like kinase (yeast) | 10.306 | 664580-664973 |
| 2050 | 2061 | engulfment and cell motility 2, ced-12 | 7.176 | 668000-668354 |
| | | homolog (C. elegans) | | |
| 2054 | 2059 | phosphoprotein enriched in astrocytes 15A | 14.429 | 669292-669690 |
| 2080 | 2049 | CLPTM1-like | 89.279 | 678524-678834 |
| 2083 | 2049 | ADP-ribosylation factor 6 | 4.368 | 679540-679784 |
| 2124 | 2034 | ras homolog gene family, member A | 135.612 | 693012-693333 |
| 2139 | 2030 | multiple endocrine neoplasia 1 | 2.911 | 698091-698430 |
| 2185 | 2015 | myelocytomatosis oncogene | 119.45 | 713438-713745 |
| 2193 | 2012 | THO complex 1 | 2.149 | 716160-716525 |
| 2199 | 2011 | autophagy-related 5 (yeast) | 7.623 | 718183-718508 |
| 2227 | 2003 | smoothened homolog (Drosophila) | 2.634 | 727770-728086 |
| 2230 | 2002 | BCL2-like 1 | 9.446 | 728838-729216 |
| 2242 | 1999 | sequestosome 1 | 51.17 | 733070-733459 |
| 2252 | 1996 | mitogen-activated protein kinase 5 | 1.378 | 736639-737018 |
| 2283 | 1987 | MAP-kinase activating death domain | 1.589 | 747015-747324 |
| 2284 | 1987 | TNF receptor associated factor 4 | 7.889 | 747325-747659 |
| 2305 | 1982 | thymoma viral proto-oncogene 1 | 21.705 | 754612-754878 |
| | | interacting protein | | |
| 2364 | 1967 | protein disulfide isomerase associated 3 | 173.82 | 774355-774677 |
| 2367 | 1966 | TSC22 domain family, member 3 | 5.809 | 775361-775690 |
| 2400 | 1959 | phosphofurin acidic cluster sorting protein 2 | 2.811 | 786380-786716 |
| 2403 | 1958 | DnaJ (Hsp40) homolog, subfamily C, | 5.417 | 787385-787676 |
| | | member 5 | | |
| 2450 | 1945 | receptor (TNFRSF)-interacting serine- | 0.965 | 803414-803712 |
| | | threonine kinase 1 | | |
| 2471 | 1940 | mutS homolog 2 (E. coli) | 6.134 | 810424-810813 |
| 2496 | 1934 | Kv channel interacting | 1.03 | 819220-819569 |
| | | protein 3, calsenilin | | |
| 2515 | 1929 | Cbp/p300-interacting transactivator, with | 22.655 | 825796-826120 |
| | | Glu/Asp-rich carboxy-terminal domain, 2 | | |
| 2547 | 1921 | cell division cycle and apoptosis regulator 1 | 1.757 | 836705-837044 |
| 2599 | 1908 | tripartite motif-containing 39 | 1.032 | 854385-854718 |
| 2608 | 1906 | E2F transcription factor 1 | 7.007 | 857154-857487 |
| 2660 | 1891 | TGFβ-regulated gene 4 | 10.934 | 874486-874847 |
| 2668 | 1890 | apoptotic chromatin condensation inducer 1 | 3.906 | 877244-877643 |
| 2670 | 1890 | BCL2-associated athanogene 3 | 5.061 | 878043-878361 |
| 2691 | 1887 | growth arrest specific 2 | 2.282 | 885284-885579 |
| 2749 | 1871 | protein phosphatase 1, regulatory | 2.369 | 905145-905540 |
| | | (inhibitor) subunit 13B | | |
| 2790 | 1859 | excision repair cross-complementing rodent | 1.408 | 919056-919386 |
| | | repair deficiency, complementation group 2 | | |
| 2811 | 1854 | retinoic acid receptor, gamma | 2.638 | 926437-926742 |
| 2815 | 1854 | serine/threonine kinase 3 (Ste20, | 4.084 | 927749-928072 |
| | | yeast homolog) | | |
| 2831 | 1851 | aldehyde dehydrogenase family 1, | 40.058 | 933071-933460 |
| | | subfamily A1 | | |
| 2838 | 1850 | catenin, beta like 1 | 20.124 | 935528-935906 |
| 2848 | 1847 | RAD9 homolog (S. pombe) | 13.395 | 938950-939251 |
| 2904 | 1829 | breast cancer 1 | 7.497 | 958124-958436 |
| 2965 | 1810 | protein kinase, DNA activated, | 0.793 | 979242-979576 |
| | | catalytic polypeptide | | |
| 3042 | 1782 | sphingosine-1-phosphate phosphatase 1 | 3.922 | 1005199-1005578 |
| 3054 | 1777 | death effector domain-containing | 1.323 | 1009277-1009659 |
| 3073 | 1775 | vanin 1 | 20.503 | 1015567-1015901 |
| 3078 | 1773 | zinc finger CCCH type containing 12A | 3.152 | 1017198-1017589 |
| 3094 | 1769 | TRAF3 interacting protein 2 | 4.391 | 1022836-1023187 |
| 3096 | 1769 | MKL (megakaryoblastic | 1.041 | 1023531-1023903 |
| | | leukemia)/myocardin-like 1 | | |
| 3234 | 1738 | FAST kinase domains 5 | 2.617 | 1071097-1071485 |
| 3268 | 1729 | B-cell leukemia/lymphoma 6 | 8.467 | 1082762-1083124 |
| 3273 | 1726 | tyrosine 3-monooxygenase/tryptophan 5- | 62.681 | 1084449-1084755 |
| | | monooxygenase activation protein, eta | | |
| | | polypeptide | | |
| 3275 | 1726 | ubiquitin-conjugating enzyme E2B, RAD6 | 13.78 | 1085017-1085315 |
| | | homology (S. cerevisiae) | | |
| 3289 | 1721 | menage a trois 1 | 12.96 | 1089606-1089959 |
| 3300 | 1718 | TNF receptor-associated factor 5 | 3.925 | 1093396-1093770 |
| 3324 | 1712 | poly-U binding splicing factor 60 | 14.514 | 1101460-1101740 |
| 3326 | 1711 | RIKEN cDNA 1200009F10 gene | 3.501 | 1102067-1102381 |
| 3338 | 1709 | NEDD8 activating enzyme E1 subunit 1 | 9.826 | 1106097-1106429 |
| 3342 | 1708 | phosphatidylinositol glycan anchor | 24.872 | 1107410-1107750 |
| | | biosynthesis, class T | | |
| 3358 | 1704 | DNA-damage regulated autophagy | 2.146 | 1112807-1113187 |
| | | modulator 1 | | |
| 3382 | 1699 | major facilitator superfamily domain | 17.753 | 1121263-1121574 |
| | | containing 10 | | |
| 3390 | 1696 | cell division cycle 2-like 1 | 17.014 | 1124002-1124331 |
| 3419 | 1688 | bladder cancer associated protein | 4.537 | 1133723-1134082 |
| | | homolog (human) | | |
| 3448 | 1680 | family with sequence similarity 188, | 2.812 | 1143475-1143791 |
| | | member A | | |
| 3499 | 1670 | SAP30 binding protein | 3.008 | 1160338-1160643 |
| 3524 | 1664 | integral membrane protein 2B | 103.29 | 1168940-1169261 |
| 3540 | 1661 | superoxide dismutase 2, mitochondrial | 2.559 | 1174163-1174529 |
| 3559 | 1654 | SKI-like | 1.243 | 1180446-1180768 |
| 3651 | 1633 | FK506 binding protein 8 | 53.498 | 1211464-1211841 |
| 3654 | 1632 | glutamate-cysteine ligase, catalytic subunit | 12.64 | 1212479-1212769 |
| 3685 | 1627 | HtrA serine peptidase 2 | 11.095 | 1222907-1223252 |
| 3692 | 1625 | family with sequence similarity 82, | 4.761 | 1225295-1225616 |
| | | member A2 | | |
| 3693 | 1624 | BCL2-associated athanogene 5 | 26.647 | 1225617-1225987 |
| 3695 | 1623 | pleiomorphic adenoma gene-like 2 | 0.74 | 1226344-1226650 |
| 3705 | 1622 | seven in absentia 2 | 1.664 | 1229814-1230210 |
| 3710 | 1621 | voltage-dependent anion channel 1 | 35.606 | 1231561-1231854 |
| 3736 | 1616 | cullin 7 | 1.583 | 1240268-1240610 |
| 3749 | 1612 | ADAMTS-like 4 | 2.67 | 1244700-1245081 |
| 3761 | 1609 | ataxia telangiectasia mutated | 0.181 | 1248864-1249255 |
| | | homolog (human) | | |
| 3776 | 1605 | death associated protein 3 | 18.724 | 1253963-1254317 |
| 3787 | 1601 | transcription factor Dp 1 | 6.434 | 1257788-1258139 |
| 3806 | 1595 | adenosine deaminase | 19.88 | 1264324-1264663 |
| 3837 | 1587 | modulator of apoptosis 1 | 2.395 | 1274626-1274921 |
| 3855 | 1584 | activating transcription factor 5 | 9.537 | 1280625-1280989 |
| 3913 | 1571 | signal transducer and activator of | 1.268 | 1299843-1300222 |
| | | transcription 5A | | |
| 3926 | 1567 | clusterin | 40.878 | 1304084-1304407 |
| 3983 | 1553 | RAS p21 protein activator 1 | 0.463 | 1323060-1323449 |
| 3994 | 1550 | caspase recruitment domain family, | 3.045 | 1326706-1327065 |
| | | member 10 | | |
| 4014 | 1545 | protein phosphatase 2 (formerly 2A), | 82.162 | 1333415-1333732 |
| | | catalytic subunit, beta isoform | | |
| 4041 | 1540 | presenilin 1 | 3.007 | 1342545-1342881 |
| 4052 | 1537 | BCL2-associated athanogene 4 | 0.353 | 1346314-1346657 |
| 4085 | 1525 | RELT tumor necrosis factor receptor | 2.067 | 1356880-1357195 |
| 4090 | 1525 | zinc finger, C3HC type 1 | 17.03 | 1358571-1358886 |
| 4106 | 1522 | TNF receptor-associated factor 2 | 7.2 | 1363971-1364287 |
| 4128 | 1517 | programmed cell death 11 | 1.078 | 1371711-1372000 |
| 4152 | 1512 | cytokine induced apoptosis inhibitor 1 | 6.495 | 1379554-1379805 |
| 4165 | 1510 | nuclear receptor subfamily 4, group A, | 3.433 | 1383906-1384203 |
| | | member 1 | | |
| 4166 | 1509 | bifunctional apoptosis regulator | 2.213 | 1384204-1384477 |
| 4199 | 1500 | cytoskeleton associated protein 2 | 1.674 | 1395624-1396011 |
| 4201 | 1500 | eukaryotic translation initiation factor 2 | 2.46 | 1396283-1396617 |
| | | alpha kinase 3 | | |
| 4202 | 1500 | intraflagellar transport 57 homolog | 4.102 | 1396618-1396929 |
| | | (Chlamydomonas) | | |
| 4247 | 1492 | B-cell receptor-associated protein 29 | 2.19 | 1411569-1411898 |
| 4250 | 1492 | caspase 9 | 1.769 | 1412589-1412860 |
| 4252 | 1491 | RRN3 RNA polymerase I transcription | 2.225 | 1413234-1413535 |
| | | factor homolog (yeast) | | |
| 4255 | 1491 | budding uninhibited by benzimidazoles 1 | 2.264 | 1414236-1414628 |
| | | homolog, β(S. cerevisiae) | | |
| 4268 | 1487 | STE20-related kinase adaptor beta | 1.082 | 1418669-1418996 |
| 4275 | 1486 | FAST kinase domains 2 | 4.522 | 1421149-1421474 |
| 4290 | 1482 | mutL homolog 1 (E. coli) | 5.514 | 1426359-1426686 |
| 4322 | 1476 | phosphatidylinositol 3-kinase, regulatory | 3.629 | 1436979-1437294 |
| | | subunit, polypeptide 2 (p85 beta) | | |
| 4325 | 1476 | eukaryotic translation elongation factor 1 2 | 3.269 | 1437945-1438305 |
| 4327 | 1475 | Notch gene homolog 2 (Drosophila) | 0.347 | 1438663-1438970 |
| 4339 | 1472 | helicase, lymphoid specific | 0.521 | 1442541-1442877 |
| 4348 | 1470 | Ras-related GTP binding A | 46.31 | 1445616-1445968 |
| 4379 | 1464 | SH3-domain GRB2-like B1 (endophilin) | 13.153 | 1455957-1456292 |
| 4383 | 1463 | tripartite motif-containing 35 | 1.003 | 1457309-1457624 |
| 4414 | 1456 | cyclin-dependent kinase 5 | 3.895 | 1467595-1467925 |
| 4421 | 1455 | ring finger protein 34 | 7.18 | 1469632-1469965 |
| 4433 | 1453 | reticulon 4 | 53.172 | 1473726-1474051 |
| 4434 | 1453 | protein kinase, interferon inducible double | 5.527 | 1474052-1474353 |
| | | stranded RNA dependent activator | | |
| 4461 | 1446 | DNA-damage-inducible transcript 4 | 3.353 | 1483293-1483590 |
| 4478 | 1441 | CCAAT/enhancer binding protein (C/EBP), β | 11.321 | 1488766-1489110 |
| 4504 | 1435 | polycomb group ring finger 2 | 3.603 | 1496993-1497354 |
| 4515 | 1433 | ceroid lipofuscinosis, neuronal 3, juvenile | 2.904 | 1500552-1500853 |
| | | (Batten, Spielmeyer-Vogt disease) | | |
| 4525 | 1431 | GATA binding protein 6 | 1.073 | 1503752-1504126 |
| 4568 | 1422 | WW domain-containing oxidoreductase | 2.113 | 1518412-1518773 |
| 4594 | 1416 | transmembrane BAX inhibitor motif | 16.969 | 1527288-1527665 |
| | | containing 6 | | |
| 4606 | 1414 | cold shock domain protein A | 171.461 | 1531366-1531649 |
| 4642 | 1405 | shisa homolog 5 (Xenopus laevis) | 11.181 | 1542721-1542999 |
| 4668 | 1399 | testis expressed gene 261 | 22.005 | 1551436-1551738 |
| 4682 | 1396 | protein phosphatase 1, regulatory | 1.002 | 1556095-1556385 |
| | | (inhibitor) subunit 13 like | | |
| 4705 | 1391 | Pleckstrin homology-like domain, family | 17.062 | 1564100-1564401 |
| | | A, member 3 | | |
| 4744 | 1384 | fibroblast growth factor receptor 1 | 0.422 | 1577052-1577365 |
| 4747 | 1383 | seven in absentia 1A | 1.166 | 1578078-1578382 |
| 4764 | 1376 | jumonji domain containing 6 | 14.926 | 1583786-1584134 |
| 4831 | 1362 | integrator complex subunit 1 | 1.012 | 1606795-1607183 |
| 4859 | 1355 | myocyte enhancer factor 2A | 0.685 | 1616416-1616715 |
| 4904 | 1346 | FAST kinase domains 3 | 2.435 | 1631671-1632058 |
| 4905 | 1346 | mitochondrial carrier homolog 1 (C. elegans) | 54.765 | 1632059-1632447 |
| 4912 | 1345 | v-Ki-ras2 Kirsten rat sarcoma viral | 3.151 | 1634477-1634773 |
| | | oncogene homolog | | |
| 4929 | 1342 | tectonic family member 3 | 1.25 | 1640228-1640526 |
| 4941 | 1339 | catenin (cadherin associated protein), β1 | 0.495 | 1644372-1644747 |
| 4944 | 1339 | B-cell leukemia/lymphoma 10 | 9.013 | 1645462-1645856 |
| 4952 | 1337 | tribbles homolog 3 (Drosophila) | 7.419 | 1648199-1648515 |
| 4953 | 1337 | mitochondrial ubiquitin ligase activator of | 2.065 | 1648516-1648850 |
| | | NFKB 1 | | |
| 4957 | 1336 | transformation related protein 53 | 6.608 | 1649857-1650157 |
| 4963 | 1334 | amyloid β(A4) precursor protein-binding, | 1.378 | 1651979-1652331 |
| | | family B, member 3 | | |
| 4993 | 1326 | collagen, type XVIII, 1 | 0.529 | 1662476-1662775 |
| 5016 | 1320 | RIKEN cDNA 4930453N24 gene | 3.737 | 1670187-1670553 |
| 5046 | 1313 | deoxyribonuclease II | 31.897 | 1680451-1680725 |
| 5047 | 1313 | estrogen receptor-binding fragment- | 21.721 | 1680726-1681024 |
| | | associated gene 9 | | |
| 5056 | 1311 | BCL2-associated athanogene 1 | 11.445 | 1683576-1683895 |
| 5079 | 1304 | baculoviral IAP repeat-containing 3 | 7.2 | 1691584-1691970 |
| 5081 | 1303 | family with sequence similarity 176, | 3.606 | 1692345-1692702 |
| | | member A | | |
| 5111 | 1298 | brain & reproductive organ-expressed protein | 57.864 | 1702336-1702627 |
| 5112 | 1297 | tumor necrosis factor, -induced protein 8 | 5.97 | 1702628-1702979 |
| 5120 | 1295 | eukaryotic translation initiation factor 5A | 661.40 | 1705373-1705736 |
| 5131 | 1292 | presenilin 2 | 2.55 | 1709139-1709525 |
| 5139 | 1291 | BCL2 binding component 3 | 1.503 | 1712045-1712425 |
| 5140 | 1291 | WD repeat domain 92 | 1.995 | 1712426-1712738 |
| 5168 | 1285 | sphingosine kinase 2 | 1.151 | 1721818-1722158 |
| 5174 | 1285 | death inducer-obliterator 1 | 1.104 | 1723982-1724350 |
| 5221 | 1275 | growth arrest & DNA-damage-inducible 45 β | 21.495 | 1740423-1740753 |
| 5222 | 1275 | BCL2/adenovirus E1B interacting protein 3 | 9.252 | 1740754-1741152 |
| 5260 | 1269 | receptor (TNFRSF)-interacting serine- | 2.702 | 1753377-1753673 |
| | | threonine kinase 2 | | |
| 5295 | 1259 | nuclear receptor subfamily 4, group A, | 0.73 | 1765734-1766070 |
| | | member 2 | | |
| 5318 | 1255 | DNA fragmentation factor, beta subunit | 1.315 | 1773243-1773629 |
| 5328 | 1252 | rhotekin | 2.833 | 1776824-1777199 |
| 5357 | 1245 | transforming growth factor, beta 1 | 13.689 | 1787146-1787456 |
| 5379 | 1240 | baculoviral IAP repeat-containing 2 | 1.473 | 1795149-1795509 |
| 5479 | 1217 | cytochrome c, somatic | 5.321 | 1830214-1830597 |
| 5501 | 1212 | microphthalmia-associated transcription factor | 0.4 | 1838060-1838448 |
| 5506 | 1211 | craniofacial development protein 1 | 17.159 | 1839938-1840310 |
| 5512 | 1210 | pleckstrin homology-like domain, family | 6.85 | 1841998-1842361 |
| | | A, member 1 | | |
| 5532 | 1204 | B-cell receptor-associated protein 31 | 116.56 | 1848903-1849265 |
| 5537 | 1202 | angiopoietin-like 4 | 0.987 | 1850651-1851035 |
| 5550 | 1200 | disintegrin & metallopeptidase domain 17 | 1.374 | 1855220-1855596 |
| 5567 | 1197 | X-linked inhibitor of apoptosis | 0.297 | 1861051-1861417 |
| 5568 | 1197 | ring finger protein 130 | 5.397 | 1861418-1861721 |
| 5589 | 1192 | Werner syndrome homolog (human) | 0.711 | 1868474-1868871 |
| 5608 | 1188 | caspase 12 | 0.856 | 1875252-1875646 |
| 5633 | 1182 | Harvey rat sarcoma virus oncogene 1 | 4.391 | 1884273-1884616 |
| 5636 | 1182 | mitogen-activated protein kinase 7 | 1.049 | 1885325-1885696 |
| 5641 | 1180 | death effector domain-containing DNA | 1.463 | 1887105-1887480 |
| | | binding protein 2 | | |
| 5649 | 1178 | STAM binding protein | 2.283 | 1889758-1890088 |
| 5663 | 1175 | CASP8 & FADD-like apoptosis regulator | 4.448 | 1894743-1895132 |
| 5671 | 1173 | programmed cell death 7 | 1.96 | 1897662-1898025 |
| 5711 | 1164 | leucine-rich & death domain containing | 2.507 | 1912080-1912460 |
| 5746 | 1157 | caspase 3 | 11.813 | 1924836-1925195 |
| 5771 | 1151 | TNFRSF1A-associated via death domain | 11.061 | 1934043-1934332 |
| 5775 | 1151 | cell death-inducing DFFA-like effector c | 55.287 | 1935411-1935807 |
| 5791 | 1147 | microtubule-associated protein 1S | 6.328 | 1941401-1941793 |
| 5844 | 1137 | BCL2-like 11 (apoptosis facilitator) | 0.584 | 1960442-1960764 |
| 5854 | 1136 | caspase 1 | 2.306 | 1964106-1964500 |
| 5862 | 1133 | zinc finger, DHHC domain containing 16 | 4.4 | 1967129-1967439 |
| 5883 | 1129 | X-ray repair complementing defective | 6.458 | 1974749-1975138 |
| | | repair in CHO cells 4 | | |
| 5906 | 1123 | sphingosine kinase 1 | 15.987 | 1983308-1983651 |
| 5931 | 1117 | Fas death domain-associated protein | 2.242 | 1992296-1992675 |
| 5946 | 1115 | diablo homolog (Drosophila) | 10.353 | 1997873-1998247 |
| 5968 | 1110 | amiloride-sensitive cation channel 1, | 1.444 | 2005882-2006234 |
| | | neuronal (degenerin) | | |
| 5987 | 1104 | ceroid-lipofuscinosis, neuronal 8 | 0.372 | 2012719-2013104 |
| 6050 | 1092 | Sp110 nuclear body protein | 2.119 | 2035393-2035780 |
| 6051 | 1092 | phosducin-like 3 | 12.75 | 2035781-2036142 |
| 6055 | 1091 | LPS-induced TN factor | 4.202 | 2037259-2037644 |
| 6056 | 1091 | programmed cell death 6 | 53.378 | 2037645-2037947 |
| 6153 | 1068 | max binding protein | 0.824 | 2073201-2073580 |
| 6165 | 1066 | G2/M-phase specific E3 ubiquitin ligase | 0.358 | 2077605-2078002 |
| 6185 | 1062 | aminoacyl tRNA synthetase complex- | 33.092 | 2084323-2084687 |
| | | interacting multifunctional protein 1 | | |
| 6223 | 1053 | glutamate-Cys ligase, modifier subunit | 22.216 | 2098389-2098782 |
| 6239 | 1049 | myocyte enhancer factor 2C | 0.524 | 2104360-2104732 |
| 6252 | 1045 | TM2 domain containing 1 | 2.155 | 2108754-2109139 |
| 6273 | 1039 | nerve growth factor | 9.393 | 2115896-2116286 |
| 6295 | 1034 | forkheadbox C1 | 0.44 | 2123858-2124256 |
| 6305 | 1031 | dual-specificity tyrosine-(Y)- | 0.52 | 2127434-2127800 |
| | | phosphorylation regulated kinase 2 | | |
| 6312 | 1030 | programmed cell death 2 | 6.216 | 2129627-2130022 |
| 6369 | 1020 | programmed cell death 4 | 0.953 | 2150178-2150561 |
| 6396 | 1013 | DNA fragmentation factor, alpha subunit | 2.45 | 2159909-2160294 |
| 6445 | 1003 | aminoacyl tRNA synthetase complex- | 12.784 | 2177473-2177849 |
| | | interacting multifunctional protein 2 | | |
| 6481 | 991 | polymerase (DNA directed), beta | 1.632 | 2190243-2190641 |
| 6522 | 984 | endonuclease G | 23.832 | 2204505-2204896 |
| 6557 | 975 | B-cell CLL/lymphoma 7C | 6.467 | 2216903-2217199 |
| 6572 | 973 | transcription factor 7, T-cell specific | 1.383 | 2222093-2222467 |
| 6624 | 964 | tumor necrosis factor receptor | 58.921 | 2240612-2240962 |
| | | superfamily, member 12a | | |
| 6644 | 960 | sema domain, transmembrane domain (TM), | 0.16 | 2247664-2248042 |
| | | and cytoplasmic domain, (semaphorin) 6A | | |
| 6647 | 960 | Bmi1 polycomb ring finger oncogene | 0.42 | 2248765-2249118 |
| 6678 | 952 | BCL2-antagonist/killer 1 | 3 | 2259855-2260161 |
| 6686 | 950 | apoptotic peptidase activating factor 1 | 0.325 | 2262408-2262743 |
| 6710 | 944 | BCL2/adenovirus E1B interacting protein 2 | 15.617 | 2270556-2270934 |
| 6736 | 938 | TNF receptor-associated factor 1 | 1.035 | 2279878-2280163 |
| 6786 | 928 | steroid receptor RNA activator 1 | 9.006 | 2297190-2297589 |
| 6798 | 926 | caspase 7 | 0.436 | 2301618-2301960 |
| 6804 | 924 | GLI-Kruppel family member GLI2 | 0.489 | 2303659-2303992 |
| 6806 | 924 | purine-nucleoside Phosphorylase 1 | 10.99 | 2304356-2304474 |
| 6807 | 923 | tumor necrosis factor receptor | 2.901 | 2304475-2304854 |
| | | superfamily, member 1a | | |
| 6813 | 922 | TNF, -induced protein 3 | 0.517 | 2306610-2306966 |
| 6830 | 918 | interleukin 19 | 4.282 | 2312386-2312719 |
| 6858 | 913 | nucleotide-binding oligomerization | 0.461 | 2322123-2322429 |
| | | domain containing 2 | | |
| 6866 | 911 | GLI-Kruppel family member GLI3 | 1.434 | 2324663-2324995 |
| 6958 | 890 | BCL2-like 12 (proline rich) | 18.291 | 2354097-2354391 |
| 6975 | 887 | yippee-like 3 (Drosophila) | 1.989 | 2359942-2360263 |
| 7010 | 877 | proteasome (prosome, macropain) | 9.024 | 2371767-2372110 |
| | | assembly chaperone 2 | | |
| 7015 | 877 | TNF (ligand) superfamily, member 12 | 4.328 | 2373485-2373776 |
| 7067 | 866 | HIV-1 tat interactive protein 2, homolog | 7.75 | 2391070-2391405 |
| | | (human) | | |
| 7082 | 863 | Pleckstrin homology domain containing, | 2.804 | 2395849-2396175 |
| | | family F (with FYVE domain) member 1 | | |
| 7092 | 861 | sirtuin 1 (silent mating type information | 0.22 | 2399178-2399470 |
| | | regulation 2, homolog) 1 (S. cerevisiae) | | |
| 7120 | 855 | caspase 6 | 4.965 | 2408466-2408843 |
| 7124 | 853 | homeodomain interacting protein kinase 2 | 0.328 | 2409808-2410107 |
| 7144 | 849 | serum/glucocorticoid regulated kinase 3 | 0.553 | 2416403-2416787 |
| 7167 | 843 | fibroblast growth factor receptor 3 | 0.243 | 2423777-2424112 |
| 7175 | 841 | baculoviral IAP repeat-containing 5 | 0.966 | 2426437-2426713 |
| 7187 | 839 | nucleotide-binding oligomerization | 0.746 | 2430407-2430803 |
| | | domain containing 1 | | |
| 7196 | 838 | transformation related protein 63 | 0.317 | 2433439-2433750 |
| 7199 | 838 | transforming growth factor, beta 3 | 1.124 | 2434410-2434754 |
| 7209 | 836 | ras homolog gene family, member B | 0.721 | 2437911-2438277 |
| 7213 | 835 | glutathione peroxidase 1 | 10.976 | 2439217-2439612 |
| 7244 | 828 | cysteine-serine-rich nuclear protein 2 | 0.278 | 2449829-2450156 |
| 7283 | 816 | ribosomal protein S6 | 18.875 | 2462245-2462567 |
| 7297 | 811 | TNF receptor-associated factor 6 | 1.188 | 2466579-2466938 |
| 7320 | 807 | C1D nuclear receptor co-repressor | 0.376 | 2474231-2474564 |
| 7349 | 800 | nucleolar protein 3 (apoptosis repressor | 2.282 | 2484064-2484342 |
| | | with CARD domain) | | |
| 7374 | 794 | ceroid-lipofuscinosis, neuronal 5 | 1.261 | 2492286-2492578 |
| 7418 | 785 | myeloid differentiation primary response | 2.514 | 2506840-2507215 |
| | | gene 116 | | |
| 7424 | 782 | RIKEN cDNA 1110007C09 gene | 3.301 | 2508903-2509183 |
| 7426 | 782 | engulfment and cell motility 1, ced-12 | 0.528 | 2509515-2509793 |
| | | homolog (C. elegans) | | |
| 7439 | 778 | B-cell leukemia/lymphoma 2 | 0.149 | 2513854-2514170 |
| 7484 | 767 | UDP-Gal: βGlcNAc β1,4- | 0.387 | 2528454-2528763 |
| | | galactosyltransferase, polypeptide 1 | | |
| 7498 | 765 | sodium channel, voltage-gated, type II, 1 | 0.184 | 2533197-2533494 |
| 7504 | 763 | interferon activated gene 204 | 9.678 | 2535305-2535372 |
| 7506 | 762 | apoptosis enhancing nuclease | 1.126 | 2535692-2536051 |
| 7528 | 756 | transmembrane protein 85 | 25.649 | 2543334-2543651 |
| 7579 | 744 | etoposide induced 2.4 mRNA | 0.629 | 2559503-2559877 |
| 7584 | 743 | apoptosis-inducing factor, mitochondrion- | 0.88 | 2561258-2561555 |
| | | associated 2 | | |
| 7596 | 740 | tumor protein, translationally-controlled 1 | 10.23 | 2565288-2565685 |
| 7631 | 732 | methyl-CpG binding domain protein 4 | 0.522 | 2576018-2576339 |
| 7651 | 725 | BH3 interacting domain death agonist | 13.705 | 2582517-2582823 |
| 7664 | 723 | distal-less homeobox 1 | 0.37 | 2586625-2586998 |
| 7672 | 721 | xeroderma pigmentosum, | 1.337 | 2589348-2589735 |
| | | complementation group A | | |
| 7675 | 720 | eukaryotic translation elongation factor 1 ε1 | 1.403 | 2590423-2590793 |
| 7689 | 717 | BCL2/adenovirus E1B interacting protein 1 | 1.953 | 2595342-2595666 |
| 7749 | 702 | peroxiredoxin 2 | 15.903 | 2616024-2616366 |
| 7784 | 693 | Ser/Thr kinase 17b (apoptosis-inducing) | 0.603 | 2627742-2628087 |
| 7794 | 691 | giant axonal neuropathy | 0.587 | 2631132-2631429 |
| 7803 | 689 | breast cancer 2 | 0.07 | 2634236-2634594 |
| 7864 | 669 | amyloid beta (A4) precursor protein- | 0.212 | 2654750-2655139 |
| | | binding, family B, member 2 | | |
| 7879 | 666 | cyclin-dependent kinase inhibitor 1A (P21) | 3.252 | 2659502-2659871 |
| 7880 | 666 | protein phosphatase 1F (PP2C domain | 2.902 | 2659872-2660259 |
| | | containing) | | |
| 7924 | 652 | excision repair cross-complementing rodent | 21.84 | 2675071-2675432 |
| | | repair deficiency, complementation group 1 | | |
| 7978 | 639 | BCL2 modifying factor | 0.17 | 2692923-2693205 |
| 8013 | 630 | TCF3 (E2A) fusion partner | 4.464 | 2704602-2704917 |
| 8026 | 624 | CASP2 and RIPK1 domain containing | 1.176 | 2709036-2709355 |
| | | adaptor with death domain | | |
| 8030 | 623 | ring finger and FYVE like domain | 0.192 | 2710236-2710629 |
| | | containing protein | | |
| 8056 | 612 | caspase 2 | 1.166 | 2718675-2719039 |
| 8065 | 610 | testis expressed gene 11 | 0.205 | 2721707-2721990 |
| 8095 | 601 | cyclin-dependent kinase inhibitor 1B | 0.381 | 2731076-2731440 |
| 8100 | 601 | E2F transcription factor 2 | 0.204 | 2732428-2732782 |
| 8116 | 595 | inhibitor of DNA binding 1 | 1.398 | 2737742-2738071 |
| 8119 | 595 | serglycin | 9.946 | 2738723-2739031 |
| 8133 | 591 | defender against cell death 1 | 4.551 | 2742894-2743239 |
| 8174 | 583 | mitochondrial ribosomal protein L41 | 0.749 | 2755819-2756155 |
| 8191 | 580 | RIKEN cDNA 2810002N01 gene | 1.368 | 2761213-2761609 |
| 8218 | 570 | interleukin 18 | 2.856 | 2769797-2770097 |
| 8241 | 562 | BCL2-associated athanogene 2 | 1.083 | 2776948-2777283 |
| 8282 | 551 | programmed cell death 5 | 3.991 | 2790756-2791058 |
| 8328 | 540 | FAST kinase domains 1 | 0.298 | 2806153-2806512 |
| 8345 | 536 | Fas (TNF receptor superfamily member 6) | 0.501 | 2812206-2812506 |
| 8349 | 535 | DNA-damage inducible transcript 3 | 4.982 | 2813622-2813956 |
| 8369 | 530 | superoxide dismutase 1, soluble | 9.577 | 2820605-2820925 |
| 8381 | 524 | nuclear protein 1 | 26.14 | 2824647-2825002 |
| 8386 | 523 | NADH dehydrogenase (ubiquinone) 1 | 1.74 | 2826135-2826503 |
| | | subcomplex, 13 | | |
| 8429 | 512 | ligase IV, DNA, ATP-dependent | 0.41 | 2841502-2841815 |
| 8473 | 502 | programmed cell death 10 | 0.375 | 2855519-2855901 |
| 8508 | 493 | serine (or cysteine) peptidase inhibitor, | 0.146 | 2867128-2867490 |
| | | clade B, member 9 | | |
| 8543 | 488 | NLR family, apoptosis inhibitory protein 1 | 0.091 | 2878738-2879123 |
| 8562 | 484 | calcium and integrin binding 1 (calmyrin) | 2.049 | 2884444-2884809 |
| 8595 | 478 | death-associated protein | 6.602 | 2895360-2895710 |
| 8608 | 475 | BCL2-interacting killer | 1.02 | 2899985-2900289 |
| 8633 | 470 | SIVA1, apoptosis-inducing factor | 2.357 | 2908717-2909086 |
| 8662 | 464 | death-associated protein kinase 3 | 0.33 | 2918007-2918383 |
| 8746 | 450 | tumor necrosis factor receptor | 0.392 | 2944708-2945036 |
| | | superfamily, member 4 | | |
| 8762 | 448 | RIKEN cDNA 1700020C11 gene | 0.321 | 2949726-2950061 |
| 8776 | 445 | TAF10 RNA polymerase II, TATA box | 1.068 | 2953967-2954306 |
| | | binding protein (TBP)-associated factor | | |
| 8785 | 442 | zinc finger protein 346 | 0.244 | 2956870-2957191 |
| 8833 | 434 | tumor necrosis factor (ligand) superfamily, | 0.089 | 2971279-2971604 |
| | | member 10 | | |
| 8911 | 415 | vitamin D receptor | 0.096 | 2993954-2994263 |
| 8917 | 414 | caspase 8 | 0.2 | 2995593-2995870 |
| 8946 | 407 | G protein-coupled receptor kinase 1 | 0.1 | 3003705-3003945 |
| 8950 | 405 | baculoviral IAP repeat-containing 6 | 0.047 | 3004660-3004919 |
| 8954 | 403 | junction-mediating and regulatory protein | 0.09 | 3005715-3006035 |
| 8970 | 400 | nuclear factor of kappa light polypeptide | 0.2 | 3010046-3010308 |
| | | gene enhancer in B-cells inhibitor, delta | | |
| 8989 | 396 | nudix (nucleoside diphosphate linked | 0.696 | 3015481-3015727 |
| | | moiety X)-type motif 2 | | |
| 8998 | 393 | BCL2-associated transcription factor 1 | 0.506 | 3017654-3017919 |
| 9019 | 388 | BCL2-associated X protein | 1.131 | 3023234-3023515 |
| 9047 | 379 | cell death-inducing DNA fragmentation | 0.326 | 3030361-3030636 |
| | | factor, alpha subunit-like effector B | | |
| 9061 | 375 | X-ray repair complementing defective | 0.116 | 3034073-3034352 |
| | | repair in Chinese hamster cells 2 | | |
| 9110 | 362 | PRKC, apoptosis, WT1, regulator | 0.2 | 3046374-3046623 |
| 9122 | 360 | BCL2-associated agonist of cell death | 0.429 | 3049436-3049721 |
| 9125 | 359 | ring finger protein 7 | 0.318 | 3050245-3050522 |
| 9151 | 352 | tumor necrosis factor receptor | 0.691 | 3056380-3056639 |
| | | superfamily, member 22 | | |
| 9168 | 347 | ribonucleotide reductase M2 B | 0.11 | 3059844-3060049 |
| | | (TP53 inducible) | | |
| 9232 | 334 | apoptosis-associated tyrosine kinase | 0.065 | 3074031-3074270 |
| 9276 | 322 | purine rich element binding protein B | 0.763 | 3083608-3083822 |
| 9291 | 319 | TP53 regulated inhibitor of apoptosis 1 | 3.404 | 3086855-3087130 |
| 9321 | 307 | cysteine-serine-rich nuclear protein 1 | 0.109 | 3093672-3093894 |
| 9351 | 299 | caspase recruitment domain family, | 0.076 | 3100085-3100282 |
| | | member 14 | | |
| 9363 | 296 | oncostatin M | 0.135 | 3102482-3102721 |
| 9386 | 291 | BCL2/adenovirus E1B 19 kD interacting | 0.168 | 3106657-3106876 |
| | | protein like | | |
| 9434 | 270 | growth arrest specific 1 | 0.093 | 3115802-3115978 |
| 9436 | 269 | Fas apoptotic inhibitory molecule | 0.408 | 3116150-3116343 |
| 9440 | 160 | NLR family, apoptosis inhibitory protein 5 | 0.618 | 3116945-3116985 |
| 9464 | 259 | DEAD (Asp-Glu-Ala-Asp) box | 0.096 | 3120923-3121116 |
| | | polypeptide 20 | | |
| 9466 | 258 | post-GPI attachment to proteins 2 | 0.218 | 3121170-3121374 |
| 9473 | 256 | engulfment and cell motility 3, ced-12 | 0.119 | 3122400-3122588 |
| | | homolog (C. elegans) | | |
| 9504 | 241 | protein Tyr phosphatase, receptor type, V | 0.04 | 3127383-3127553 |
| 9508 | 240 | fission 1 (mitochondrial outer membrane) | 0.298 | 3127965-3128127 |
| | | homolog (yeast) | | |
| 9516 | 238 | nerve growth factor receptor (TNFRSF16) | 0.256 | 3129086-3129263 |
| | | associated protein 1 | | |
| 9517 | 238 | mucosa associated lymphoid tissue | 0.359 | 3129264-3129311 |
| | | lymphoma translocation gene 1 | | |
| 9526 | 234 | NUAK family, SNF1-like kinase, 2 | 0.077 | 3130443-3130616 |
| 9547 | 229 | Ras association (RalGDS/AF-6) domain | 0.163 | 3133777-3133906 |
| | | family member 5 | | |
| 9576 | 215 | tumor necrosis factor receptor | 0.089 | 3137352-3137413 |
| | | superfamily, member 10b | | |
| 9587 | 211 | tensin 4 | 0.089 | 3138556-3138633 |
| 9679 | 173 | heat shock protein 1B | 0.091 | 3147029-3147080 |
| 9740 | 139 | betacellulin, epidermal growth factor | 0.073 | 3150839-3150877 |
| | | family member | | |
| 9741 | 139 | NLR family, pyrin domain containing 3 | 0.035 | 3150878-3150975 |
| 3157184 | 1487 | retinoic acid receptor, beta | 1.024 | 3177484-3177583 |
| 3157219 | 274 | eyes absent 1 homolog (Drosophila) | 0.064 | 3260105-3260204 |
| 3157247 | 594 | endoplasmic reticulum (ER) to nucleus | 0.18 | 3179284-3179383 |
| | | signalling 1 | | |
| 3157277 | 397 | cell death-inducing DNA fragmentation | 0.341 | 3274796-3274895 |
| | | factor, -like effector A | | |
| 3157296 | 3494 | RNA binding motif protein 25 | 4.319 | 3267605-3267704 |
| 3157366 | 450 | angiotensinogen (serpin peptidase | 0.242 | 3260305-3260404 |
| | | inhibitor, clade A, member 8) | | |
| 3157479 | 733 | ELL associated factor 2 | 0.451 | 3264005-3264104 |
| 3157505 | 644 | crystallin, alpha B | 0.99 | 3280749-3280848 |
| 3157518 | 901 | ectodysplasin A2 isoform receptor | 0.239 | 3181584-3181683 |
| 3157545 | 387 | death-associated protein kinase 2 | 0.216 | 3254417-3254516 |
| 3157559 | 371 | XIAP associated factor 1 | 0.143 | 3203397-3203496 |
| 3157562 | 1064 | NLR family, pyrin domain containing 1A | 0.283 | 3194871-3194970 |
| 3157570 | 321 | relaxin/insulin-like family peptide receptor 2 | 0.161 | 3227917-3228016 |
| 3157594 | 236 | LIM homeobox transcription factor 1 beta | 0.194 | 3202097-3202196 |
| 3157643 | 549 | zinc finger CCCH type containing 8 | 0.373 | 3219691-3219790 |
| 3157762 | 794 | APAF1 interacting protein | 6.754 | 3227717-3227816 |
| 3157765 | 1398 | twist homolog 1 (Drosophila) | 3.622 | 3160121-3160220 |
| 3157772 | 2098 | RIKEN cDNA 2610301G19 gene | 2.281 | 3173184-3173283 |
| 3157807 | 762 | src homology 2 domain-containing | 0.391 | 3186971-3187070 |
| | | transforming protein B | | |
| 3157837 | 1573 | caspase 8 associated protein 2 | 0.5 | 3184971-3185070 |
| 3157885 | 1542 | sema domain, Immunoglobulin domain | 0.705 | 3168184-3168283 |
| | | (Ig), short basic domain, secreted, | | |
| | | (semaphorin) 3A | | |
| 3157890 | 1059 | angiotensin II receptor, type 2 | 0.668 | 3167484-3167583 |
| 3157913 | 2302 | topoisomerase I binding, arginine/serine- | 2.01 | 3173284-3173383 |
| | | rich | | |
| 3157926 | 837 | NA | 0.212 | 3253017-3253116 |
| 3157949 | 477 | protein C | 0.42 | 3271796-3271895 |
| 3157952 | 795 | homeobox, msh-like 1 | 0.717 | 3279749-3279848 |
| 3157977 | 1031 | interleukin 7 | 0.642 | 3242917-3243016 |
| 3157980 | 428 | phospholipase C, gamma 2 | 0.099 | 3169484-3169583 |
| 3157993 | 162 | epidermal growth factor receptor | 0.048 | 3166784-3166883 |
| 3158019 | 362 | ABO blood group (transferase A, 1-3-N- | 0.204 | 3185571-3185670 |
| | | acetylgalactosaminyltransferase, transferase B, | | |
| | | 1-3-galactosyltransferase) | | |
| 3158038 | 176 | Fc receptor, IgE, high affinity I, γpolypeptide | 0.258 | 3201197-3201296 |
| 3158091 | 478 | NLR family, CARD domain containing 4 | 0.179 | 3216191-3216290 |
| 3158094 | 886 | forkhead box O3 | 0.446 | 3175484-3175583 |
| 3158120 | 566 | gasdermin A | 0.477 | 3209058-3209157 |
| 3158121 | 3735 | transformation related protein 53 inducible | 2.567 | 3197071-3197170 |
| | | nuclear protein 1 | | |
| 3158129 | 525 | protein Tyr phosphatase, receptor type, F | 0.136 | 3255205-3255304 |
| 3158132 | 612 | RIKEN cDNA 4632434I11 gene | 0.26 | 3275096-3275195 |
| 3158149 | 629 | Src homology 2 domain containing F | 0.416 | 3221791-3221890 |
| 3158154 | 347 | microtubule-associated protein tau | 0.08 | 3245217-3245316 |
| 3158175 | 190 | excision repair cross-complementing rodent | 0.023 | 3230317-3230416 |
| | | repair deficiency, complementation group 6 | | |
| 3158199 | 521 | hepatocyte growth factor | 0.226 | 3253417-3253516 |
| 3158202 | 2263 | GULP, engulfment adaptor PTB domain | 3.671 | 3167784-3167883 |
| | | containing 1 | | |
| 3158294 | 648 | matrix metallopeptidase 2 | 0.413 | 3214291-3214390 |
| 3158322 | 490 | NLR family, apoptosis inhibitory protein 2 | 0.102 | 3179584-3179683 |
| 3158324 | 937 | apoptosis, caspase activation inhibitor | 1.828 | 3272696-3272795 |
| 3158331 | 982 | NEL-like 1 (chicken) | 0.565 | 3163221-3163320 |
| 3158359 | 394 | angiotensin II receptor, type 1a | 0.175 | 3213058-3213157 |
| 3158381 | 762 | CD24a antigen | 0.906 | 3245917-3246016 |

TABLE 15
Protein folding (Chinese hamster)

| SEQ | | | Avg | siRNA |
|---|---|---|---|---|
| ID NO: | consL | Description | Cov | SEQ ID NOs: |
| 91 | 3840 | peptidyl-prolyl isomerase G (cyclophilin G) | 10.266 | 38781-39067 |
| 164 | 3470 | calnexin | 23.27 | 61559-61785 |
| 218 | 3290 | alanyl-tRNA synthetase | 25.07 | 77662-77970 |
| 412 | 2946 | DnaJ (Hsp40) homolog, subfamily C, | 7.271 | 133746-134002 |
| | | member 14 | | |
| 476 | 2865 | heat shock 105 kDa/110 kDa protein 1 | 19.863 | 151195-151420 |
| 546 | 2787 | DnaJ (Hsp40) homolog, subfamily C, | 22.023 | 171304-171555 |
| | | member 10 | | |
| 579 | 2758 | heat shock protein 90, beta (Grp94), | 606.207 | 180574-180954 |
| | | member 1 | | |
| 594 | 2744 | heat shock protein 90, alpha (cytosolic), | 93.844 | 184698-184927 |
| | | class A member 1 | | |
| 827 | 2572 | heat shock protein 9 | 28.56 | 255926-256325 |
| 893 | 2541 | DnaJ (Hsp40) homolog, subfamily A, | 15.853 | 276519-276904 |
| | | member 2 | | |
| 977 | 2496 | heat shock protein 90 alpha (cytosolic), | 609.471 | 304274-304591 |
| | | class B member 1 | | |
| 1048 | 2451 | RAN binding protein 2 | 3.802 | 328313-328601 |
| 1078 | 2437 | ERO1-like (S. cerevisiae) | 6.094 | 338047-338432 |
| 1097 | 2428 | sarcolemma associated protein | 1.377 | 344524-344917 |
| 1254 | 2355 | expressed sequence C80913 | 4.935 | 397171-397493 |
| 1384 | 2293 | TNF receptor-associated protein 1 | 66.179 | 441242-441639 |
| 1543 | 2232 | heat shock protein 1 (chaperonin) | 134.366 | 494743-495086 |
| 1679 | 2181 | FK506 binding protein 4 | 66.756 | 541802-542184 |
| 1925 | 2104 | DnaJ (Hsp40) homolog, subfamily A, | 15.15 | 625909-626254 |
| | | member 3 | | |
| 1932 | 2102 | DnaJ (Hsp40) homolog, subfamily A, | 18.764 | 628385-628725 |
| | | member 1 | | |
| 1948 | 2092 | t-complex protein 1 | 67.336 | 633771-634149 |
| 1960 | 2089 | DnaJ (Hsp40) homolog, subfamily C, | 1.225 | 637892-638209 |
| | | member 16 | | |
| 2029 | 2068 | heat shock protein 8 | 891.015 | 660889-661277 |
| 2076 | 2052 | DnaJ (Hsp40) homolog, subfamily B, | 9.75 | 677203-677558 |
| | | member 1 | | |
| 2198 | 2012 | FK506 binding protein 9 | 6.327 | 717817-718182 |
| 2403 | 1958 | DnaJ (Hsp40) homolog, subfamily C, | 5.417 | 787385-787676 |
| | | member 5 | | |
| 2408 | 1957 | chaperonin containing Tcp1, subunit 3 (γ) | 229.706 | 789130-789474 |
| 2502 | 1933 | chaperonin containing Tcp1, subunit 2 (β) | 197.327 | 821357-821658 |
| 2610 | 1905 | FK506 binding protein 10 | 11.722 | 857806-858195 |
| 2671 | 1890 | chaperonin containing Tcp1, 4 (δ) | 106.158 | 878362-878726 |
| 2722 | 1877 | calreticulin | 630.596 | 895691-896051 |
| 2995 | 1797 | chaperonin containing Tcp1, 6a (zeta) | 101.293 | 989555-989847 |
| 3064 | 1776 | chaperonin containing Tcp1, 7 (eta) | 197.813 | 1012622-1013001 |
| 3202 | 1747 | chaperonin containing Tcp1, 8 (theta) | 46.504 | 1060416-1060692 |
| 3243 | 1737 | peptidylprolyl isomerase (cyclophilin)-like 4 | 2.479 | 1074139-1074475 |
| 3263 | 1730 | tubulin-specific chaperone E | 13.488 | 1080945-1081272 |
| 3269 | 1729 | chaperonin containing Tcp1, subunit 5 (ε) | 174.058 | 1083125-1083449 |
| 3276 | 1726 | peptidylprolyl isomerase domain and WD | 1.901 | 1085316-1085607 |
| | | repeat containing 1 | | |
| 3399 | 1693 | peptidylprolyl isomerase (cyclophilin)-like 2 | 8.8 | 1127061-1127426 |
| 3651 | 1633 | FK506 binding protein 8 | 53.498 | 1211464-1211841 |
| 3768 | 1607 | protein (peptidyl-prolyl cis/trans | 5.639 | 1251267-1251627 |
| | | isomerase) NIMA-interacting 1 | | |
| 3893 | 1575 | FK506 binding protein 1a | 280.554 | 1293334-1293698 |
| 4000 | 1549 | von Hippel-Lindau binding protein 1 | 35.144 | 1328790-1329108 |
| 4159 | 1510 | DnaJ (Hsp40) homolog, subfamily C, | 3.933 | 1381932-1382211 |
| | | member 1 | | |
| 4267 | 1487 | STIP1 homology and U-Box containing | 36.452 | 1418307-1418668 |
| | | protein 1 | | |
| 4379 | 1464 | SH3-domain GRB2-like B1 (endophilin) | 13.153 | 1455957-1456292 |
| 4393 | 1460 | caseinolytic peptidase X (E. coli) | 1.978 | 1460653-1461024 |
| 4429 | 1454 | GrpE-like 1, mitochondrial | 12.051 | 1472389-1472681 |
| 4545 | 1426 | GrpE-like 2, mitochondrial | 1.493 | 1510687-1510976 |
| 4697 | 1393 | torsin family 1, member A (torsin A) | 20.451 | 1561330-1561725 |
| 4955 | 1336 | peptidylprolyl isomerase D (cyclophilin D) | 17.796 | 1649170-1649515 |
| 5149 | 1289 | DnaJ (Hsp40) homolog, subfamily B, | 0.929 | 1715305-1715623 |
| | | member 9 | | |
| 5217 | 1277 | FK506 binding protein 5 | 0.441 | 1738906-1739301 |
| 5227 | 1274 | selenoprotein | 61.456 | 1742333-1742644 |
| 5347 | 1248 | DnaJ (Hsp40) homolog, subfamily B, | 3.209 | 1783440-1783810 |
| | | member 12 | | |
| 5350 | 1247 | DnaJ (Hsp40) homolog, subfamily B, | 17.061 | 1784585-1784897 |
| | | member 11 | | |
| 5405 | 1236 | DnaJ (Hsp40) homolog, subfamily B, | 1.568 | 1804161-1804465 |
| | | member 4 | | |
| 5852 | 1136 | aryl-hydrocarbon receptor-interacting protein | 21.695 | 1963346-1963707 |
| 5965 | 1111 | natural killer tumor recognition sequence | 0.378 | 2004821-2005182 |
| 6059 | 1090 | torsin family 2, member A | 4.118 | 2038737-2039067 |
| 6183 | 1062 | FK506 binding protein 14 | 2.059 | 2083548-2083925 |
| 6388 | 1016 | serologically defined colon cancer | 3.1 | 2157023-2157404 |
| | | antigen 10 | | |
| 6631 | 962 | DnaJ (Hsp40) homolog, subfamily C, | 1.346 | 2243108-2243387 |
| | | member 17 | | |
| 6640 | 960 | calreticulin 3 | 3.271 | 2246344-2246668 |
| 6648 | 959 | DnaJ (Hsp40) homolog, subfamily C, | 1.456 | 2249119-2249439 |
| | | member 30 | | |
| 6662 | 956 | peptidylprolyl isomerase C | 21.193 | 2253978-2254373 |
| 6684 | 951 | peptidylprolyl isomerase B | 30.861 | 2261765-2262058 |
| 6723 | 941 | peptidylprolyl isomerase E (cyclophilin E) | 11.137 | 2275330-2275633 |
| 7277 | 817 | DnaJ (Hsp40) homolog, subfamily C, | 0.36 | 2460206-2460591 |
| | | member 18 | | |
| 7348 | 800 | DnaJ (Hsp40) homolog, subfamily C, | 4.236 | 2483678-2484063 |
| | | member 4 | | |
| 7499 | 764 | FK506 binding protein 11 | 6.2 | 2533495-2533867 |
| 7597 | 740 | prefoldin 2 | 8.764 | 2565686-2566071 |
| 7599 | 740 | FK506 binding protein 7 | 1.092 | 2566115-2566476 |
| 7642 | 729 | peptidylprolyl isomerase A | 86.046 | 2579547-2579908 |
| 7643 | 729 | FK506 binding protein 3 | 38.663 | 2579909-2580256 |
| 7889 | 664 | ubiquitously expressed transcript | 1.147 | 2662979-2663371 |
| 8128 | 593 | DnaJ (Hsp40) homolog, subfamily B, | 0.47 | 2741242-2741540 |
| | | member 5 | | |
| 8339 | 538 | FK506 binding protein 2 | 5.81 | 2810112-2810427 |
| 8366 | 531 | prefoldin 5 | 2.394 | 2819456-2819825 |
| 8398 | 520 | cell division cycle 26 | 6.939 | 2830505-2830878 |
| 8405 | 517 | heat shock protein 1 (chaperonin 10) | 4.477 | 2833031-2833420 |
| 8480 | 501 | peptidylprolyl isomerase (cyclophilin)-like 1 | 0.94 | 2857424-2857802 |
| 8689 | 461 | prefoldin 1 | 2.791 | 2926689-2926987 |
| 8788 | 442 | tetratricopeptide repeat domain 9C | 0.133 | 2957757-2958131 |
| 8881 | 421 | protein (peptidyl-prolyl cis/trans isomerase) | 1.474 | 2985485-2985777 |
| | | NIMA-interacting, 4 (parvulin) | | |
| 8886 | 420 | H2-K region expressed gene 2 | 4.724 | 2986944-2987208 |
| 8901 | 416 | RIKEN cDNA A830007P12 gene | 0.129 | 2991112-2991407 |
| 8963 | 401 | FK506 binding protein 1b | 0.504 | 3008274-3008544 |
| 9430 | 271 | peptidyl prolyl isomerase H | 0.124 | 3115010-3115199 |
| 3157256 | 387 | peptidylprolyl isomerase (cyclophilin)-like 3 | 1.624 | 3262205-3262304 |
| 3157418 | 441 | DnaJ (Hsp40) homolog, subfamily B, | 0.238 | 3228617-3228716 |
| | | member 14 | | |
| 3157499 | 462 | FK506 binding protein 6 | 0.331 | 3177284-3177383 |
| 3157505 | 644 | crystallin, alpha B | 0.99 | 3280749-3280848 |
| 3157831 | 1176 | FK506 binding protein 15 | 0.713 | 3167384-3167483 |
| 3157871 | 528 | DnaJ (Hsp40) homolog, subfamily A, | 0.656 | 3215391-3215490 |
| | | member 4 | | |
| 3158190 | 691 | histocompatibility 2, class II, locus Mb2 | 2.388 | 3199171-3199196 |
| 3158259 | 974 | histocompatibility 2, class II, locus Mb1 | 2.25 | 3256605-3256704 |
| 3158293 | 407 | chaperonin containing Tcp1, 6b (zeta) | 0.228 | 3166684-3166783 |

TABLE 16
Immune response (Chinese hamster)

| SEQ | | | Avg | siRNA |
|---|---|---|---|---|
| ID NO: | consL | Description | Cov | SEQ ID NOs: |
| 73 | 3972 | cell adhesion molecule 1 | 13.147 | 32944-33332 |
| 78 | 3935 | strawberry notch homolog 2 (Drosophila) | 39.592 | 34611-34972 |
| 440 | 2902 | toll interacting protein | 9.02 | 141719-141960 |
| 680 | 2676 | polymerase (RNA) III (DNA directed) | 5.84 | 211082-211316 |
| | | polypeptide E | | |
| 1175 | 2393 | inositol polyphosphate phosphatase-like 1 | 3.628 | 371083-371386 |
| 1299 | 2335 | toll-like receptor 4 | 2.692 | 412131-412513 |
| 1330 | 2323 | complement component 1, r subcomponent | 62.586 | 422509-422751 |
| 1382 | 2293 | CD276 antigen | 2.822 | 440554-440858 |
| 1440 | 2270 | TANK-binding kinase 1 | 3.946 | 460287-460685 |
| 1490 | 2250 | transcription factor E3 | 4.882 | 477308-477628 |
| 1601 | 2208 | complement component 1, s subcomponent | 7.355 | 514675-514999 |
| 1694 | 2176 | toll-like receptor 2 | 12.948 | 547130-547467 |
| 1703 | 2174 | endoplasmic reticulum aminopeptidase 1 | 16.062 | 550016-550337 |
| 1718 | 2169 | MAD homolog 3 (Drosophila) | 1.913 | 555364-555694 |
| 1873 | 2121 | protein kinase C, delta | 15.233 | 608454-608757 |
| 1885 | 2116 | interleukin-1 receptor-associated kinase 1 | 6.896 | 612534-612817 |
| 1980 | 2085 | complement component 1, r subcomponent B | 28.837 | 644971-645023 |
| 2234 | 2001 | signal transducer and activator of | 2.945 | 730267-730586 |
| | | transcription 6 | | |
| 2242 | 1999 | sequestosome 1 | 51.17 | 733070-733459 |
| 2471 | 1940 | mutS homolog 2 (E. coli) | 6.134 | 810424-810813 |
| 2474 | 1940 | epiregulin | 9.501 | 811533-811821 |
| 2477 | 1938 | complement component factor h | 1.484 | 812520-812875 |
| 2520 | 1929 | drebrin-like | 40.69 | 827385-827727 |
| 2525 | 1927 | myxovirus (influenza virus) resistance 2 | 8.118 | 829145-829432 |
| 2627 | 1901 | tubulointerstitial nephritis antigen-like 1 | 471.92 | 863337-863698 |
| 2876 | 1838 | transporter 2, ATP-binding cassette, | 14.82 | 948495-948800 |
| | | subfamily B (MDR/TAP) | | |
| 3073 | 1775 | vanin 1 | 20.50 | 1015567-1015901 |
| 3094 | 1769 | TRAF3 interacting protein 2 | 4.391 | 1022836-1023187 |
| 3179 | 1750 | polymerase (RNA) III (DNA directed) | 5.685 | 1052412-1052729 |
| | | polypeptide D | | |
| 3259 | 1732 | polymerase (RNA) III (DNA directed) | 15.023 | 1079448-1079786 |
| | | polypeptide C | | |
| 3268 | 1729 | B-cell leukemia/lymphoma 6 | 8.467 | 1082762-1083124 |
| 3603 | 1645 | Fc receptor, IgG, alpha chain transporter | 84.176 | 1195070-1195378 |
| 3771 | 1606 | ectonucleotide | 1.076 | 1252246-1252538 |
| | | pyrophosphatase/phosphodiesterase 1 | | |
| 3936 | 1565 | predicted gene 5077 | 4.951 | 1307451-1307521 |
| 4041 | 1540 | presenilin 1 | 3.007 | 1342545-1342881 |
| 4063 | 1533 | transporter 1, ATP-binding cassette, | 4.595 | 1349852-1350157 |
| | | subfamily B (MDR/TAP) | | |
| 4126 | 1519 | avian reticuloendotheliosis viral (v-rel) | 4.305 | 1371109-1371427 |
| | | oncogene related B | | |
| 4240 | 1493 | complement factor properdin | 2.075 | 1409395-1409692 |
| 4256 | 1491 | polymerase (RNA) III (DNA directed) | 1.005 | 1414629-1414949 |
| | | polypeptide B | | |
| 4290 | 1482 | mutL homolog 1 (E. coli) | 5.514 | 1426359-1426686 |
| 4513 | 1433 | leukemia inhibitory factor | 2.095 | 1499872-1500182 |
| 4578 | 1419 | 2′-5′ oligoadenylate synthetase-like 2 | 1.78 | 1521814-1522122 |
| 4619 | 1411 | major facilitator superfamily domain | 1.657 | 1535249-1535610 |
| | | containing 6 | | |
| 4662 | 1400 | transcription factor EB | 2.445 | 1549445-1549837 |
| 4780 | 1372 | CCAAT/enhancer binding protein (C/EBP), γ | 0.522 | 1588969-1589358 |
| 4832 | 1362 | mitochondrial antiviral signaling protein | 1.615 | 1607184-1607527 |
| 4944 | 1339 | B-cell leukemia/lymphoma 10 | 9.013 | 1645462-1645856 |
| 4957 | 1336 | transformation related protein 53 | 6.608 | 1649857-1650157 |
| 5102 | 1300 | complement component (3b/4b) receptor 1-like | 36.058 | 1699537-1699891 |
| 5103 | 1300 | histocompatibility 2, D region locus 1 | 14.507 | 1699892-1699970 |
| 5114 | 1296 | ECSIT homolog (Drosophila) | 34.83 | 1703363-1703719 |
| 5131 | 1292 | presenilin 2 | 2.55 | 1709139-1709525 |
| 5154 | 1287 | solute carrier family 11 (proton-coupled | 2.617 | 1716973-1717346 |
| | | divalent metal ion transporters), member 1 | | |
| 5189 | 1282 | OTU domain, ubiquitin aldehyde binding 1 | 6.598 | 1729190-1729552 |
| 5233 | 1274 | histocompatibility 2, K1, K region | 12.62 | 1744314-1744510 |
| 5244 | 1272 | interleukin 4 receptor, alpha | 1.087 | 1748021-1748398 |
| 5260 | 1269 | receptor (TNFRSF)-interacting serine- | 2.702 | 1753377-1753673 |
| | | threonine kinase 2 | | |
| 5436 | 1229 | polymerase (RNA) III (DNA directed) | 0.45 | 1814931-1815240 |
| | | polypeptide F | | |
| 5532 | 1204 | B-cell receptor-associated protein 31 | 116.56 | 1848903-1849265 |
| 5598 | 1190 | parathymosin | 27.95 | 1871721-1872006 |
| 5618 | 1187 | myeloid differentiation primary response | 1.629 | 1878827-1879137 |
| | | gene 88 | | |
| 5644 | 1179 | complement component 3 | 0.472 | 1888266-1888655 |
| 5825 | 1141 | ORAI calcium release-activated calcium | 3.196 | 1953406-1953799 |
| | | modulator 1 | | |
| 5948 | 1114 | interferon regulatory factor 7 | 2.718 | 1998635-1999022 |
| 5964 | 1111 | colony stimulating factor 3 (granulocyte) | 2.413 | 2004485-2004820 |
| 6050 | 1092 | Sp110 nuclear body protein | 2.119 | 2035393-2035780 |
| 6073 | 1086 | histocompatibility 2, Q region locus 10 | 6.325 | 2043884-2044062 |
| 6124 | 1073 | linker for activation of T cells | 2.661 | 2062427-2062767 |
| 6240 | 1048 | canopy 3 homolog (zebrafish) | 15.161 | 2104733-2105122 |
| 6334 | 1028 | chemokine (C-X-C motif) ligand 12 | 0.641 | 2137589-2137972 |
| 6418 | 1008 | histocompatibility 2, T region locus 23 | 35.314 | 2167964-2168216 |
| 6454 | 999 | toll-interleukin 1 receptor (TIR) domain- | 0.575 | 2180459-2180745 |
| | | containing adaptor protein | | |
| 6507 | 986 | acid phosphatase 5, tartrate resistant | 9.561 | 2199344-2199734 |
| 6550 | 978 | Nedd4 family interacting protein 1 | 41.452 | 2214566-2214874 |
| 6615 | 966 | histocompatibility 2, Q region locus 7 | 6.966 | 2237589-2237640 |
| 6647 | 960 | Bmi1 polycomb ring finger oncogene | 0.42 | 2248765-2249118 |
| 6745 | 936 | proteasome (prosome, macropain) subunit, β | 32.531 | 2282619-2282981 |
| | | type 8 (large multifunctional peptidase 7) | | |
| 6858 | 913 | nucleotide-binding oligomerization domain | 0.461 | 2322123-2322429 |
| | | containing 2 | | |
| 6916 | 900 | membrane-associated ring finger (C3HC4) 8 | 0.75 | 2340263-2340589 |
| 7015 | 877 | tumor necrosis factor (ligand) superfamily, | 4.328 | 2373485-2373776 |
| | | member 12 | | |
| 7039 | 872 | Fc receptor, IgG, low affinity III | 2.956 | 2381692-2381999 |
| 7128 | 852 | SAM domain and HD domain, 1 | 0.214 | 2411159-2411550 |
| 7135 | 850 | DNA cross-link repair 1C, PSO2 homolog | 0.264 | 2413358-2413664 |
| | | (S. cerevisiae) | | |
| 7223 | 833 | chemokine (C-X-C motif) ligand 1 | 3.826 | 2442608-2443003 |
| 7260 | 823 | DEAD (Asp-Glu-Ala-Asp) box polypeptide 58 | 0.166 | 2454994-2455378 |
| 7283 | 816 | ribosomal protein S6 | 18.875 | 2462245-2462567 |
| 7297 | 811 | TNF receptor-associated factor 6 | 1.188 | 2466579-2466938 |
| 7469 | 770 | CD1d1 antigen | 0.505 | 2523514-2523656 |
| 7586 | 743 | phosphoprotein associated with | 0.439 | 2561944-2562307 |
| | | glycosphingolipid microdomains 1 | | |
| 7670 | 721 | myxovirus (influenza virus) resistance 1 | 0.687 | 2588615-2588951 |
| 7676 | 720 | chemokine (C-C motif) ligand 2 | 14.55 | 2590794-2591157 |
| 7683 | 718 | toll-like receptor 3 | 0.226 | 2593179-2593525 |
| 7716 | 710 | polymerase (RNA) III (DNA directed) | 2.352 | 2604412-2604804 |
| | | polypeptide H | | |
| 7754 | 701 | hemochromatosis | 0.638 | 2617430-2617793 |
| 7764 | 698 | polymerase (RNA) III (DNA directed) | 0.231 | 2620918-2621272 |
| | | polypeptide G | | |
| 7874 | 666 | CD1d2 antigen | 0.935 | 2658252-2658336 |
| 7903 | 658 | interleukin 1 receptor accessory protein | 0.254 | 2667913-2668256 |
| 7929 | 651 | interleukin 23, alpha subunit p19 | 0.852 | 2676772-2677097 |
| 7943 | 647 | proteasome maturation protein | 19.088 | 2681546-2681896 |
| 8097 | 601 | histocompatibility 2, Q region locus 2 | 1.764 | 2731750-2731823 |
| 8129 | 592 | exonuclease 1 | 0.312 | 2741541-2741842 |
| 8218 | 570 | interleukin 18 | 2.856 | 2769797-2770097 |
| 8244 | 562 | interleukin 1 receptor-like 1 | 0.299 | 2777898-2778255 |
| 8245 | 562 | calcitonin gene-related peptide-receptor | 0.987 | 2778256-2778534 |
| | | component protein | | |
| 8304 | 546 | macrophage migration inhibitory factor | 43.469 | 2798316-2798434 |
| 8312 | 543 | immunoglobulin joining chain | 0.441 | 2800818-2801142 |
| 8318 | 541 | T-cell specific GTPase | 0.193 | 2802893-2803167 |
| 8345 | 536 | Fas (TNF receptor superfamily member 6) | 0.501 | 2812206-2812506 |
| 8495 | 496 | SH2B adaptor protein 2 | 0.174 | 2862373-2862711 |
| 8504 | 494 | chemokine (C-X-C motif) ligand 10 | 1.586 | 2865648-2866015 |
| 8531 | 490 | interleukin 15 | 1.901 | 2874576-2874952 |
| 8597 | 477 | mannan-binding lectin serine peptidase 2 | 0.156 | 2896069-2896411 |
| 8616 | 474 | Src-like-adaptor 2 | 1.772 | 2902824-2903199 |
| 8663 | 464 | chemokine (C-C motif) receptor 7 | 0.236 | 2918384-2918739 |
| 8696 | 459 | CSF 2 (granulocyte-macrophage) | 1.109 | 2928757-2929061 |
| 8719 | 455 | histocompatibility 28 | 0.469 | 2936057-2936444 |
| 8794 | 441 | histocompatibility 2, Q region locus 1 | 1.023 | 2959862-2959912 |
| 8812 | 439 | TNF (ligand) superfamily, member 9 | 11.755 | 2964694-2965039 |
| 8833 | 434 | TNF (ligand) superfamily, member 10 | 0.089 | 2971279-2971604 |
| 8871 | 423 | spondin 2, extracellular matrix protein | 0.189 | 2982359-2982686 |
| 9014 | 389 | polymerase (RNA) III (DNA directed) | 0.509 | 3021834-3022134 |
| | | polypeptide K | | |
| 9021 | 387 | hemopexin | 0.262 | 3023816-3024122 |
| 9064 | 373 | complement component 8, | 0.685 | 3034878-3035143 |
| | | gamma polypeptide | | |
| 9067 | 373 | proteasome (prosome, macropain), (3 type 9 | 0.464 | 3035689-3035987 |
| | | (large multifunctional peptidase 2) | | |
| 9135 | 356 | interleukin 1 receptor, type I | 0.507 | 3052757-3052969 |
| 9164 | 348 | TNF (ligand) superfamily, member 11 | 0.157 | 3058993-3059213 |
| 9204 | 341 | POU domain, class 2, transcription factor 2 | 0.107 | 3068222-3068455 |
| 9363 | 296 | oncostatin M | 0.135 | 3102482-3102721 |
| 9367 | 295 | Fc receptor, IgG, low affinity IIb | 0.189 | 3103313-3103351 |
| 9389 | 289 | TNF (ligand) superfamily, member 4 | 0.18 | 3107093-3107318 |
| 9395 | 285 | 2′-5′ oligoadenylate synthetase 1B | 0.156 | 3108340-3108557 |
| 9517 | 238 | mucosa associated lymphoid tissue | 0.359 | 3129264-3129311 |
| | | lymphoma translocation gene 1 | | |
| 9611 | 202 | chemokine (C-C motif) ligand 9 | 0.268 | 3141032-3141071 |
| 9624 | 196 | toll-like receptor 13 | 0.061 | 3142028-3142161 |
| 9667 | 178 | chemokine (C-C motif) receptor 2 | 0.144 | 3146072-3146098 |
| 9670 | 175 | histocompatibility 2, Q region locus 8 | 0.302 | 3146338-3146451 |
| 9720 | 149 | chemokine (C-X-C motif) ligand 3 | 0.148 | 3149776-3149850 |
| 3157279 | 427 | ectonucleotide pyrophosphatase/ | 0.153 | 3182184-3182283 |
| | | phosphodiesterase 3 | | |
| 3157459 | 250 | chemokine (C-C motif) ligand 11 | 0.299 | 3199071-3199170 |
| 3157520 | 492 | complement component 1, r subcomponent-like | 0.264 | 3224791-3224890 |
| 3157558 | 742 | chemokine (C-C motif) ligand 7 | 6.395 | 3279849-3279948 |
| 3157639 | 212 | spleen tyrosine kinase | 0.041 | 3259705-3259804 |
| 3157663 | 437 | interleukin 1 receptor-like 2 | 0.224 | 3176584-3176683 |
| 3157759 | 437 | toll-like receptor 1 | 0.309 | 3203997-3204096 |
| 3157859 | 973 | Casitas B-lineage lymphoma b | 0.221 | 3172084-3172183 |
| 3157977 | 1031 | interleukin 7 | 0.642 | 3242917-3243016 |
| 3158027 | 1030 | akirin 2 | 2.138 | 3188971-3189070 |
| 3158038 | 176 | Fc receptor, IgE, high affinity I, γ polypeptide | 0.258 | 3201197-3201296 |
| 3158135 | 418 | mannan-binding lectin serine peptidase 1 | 0.152 | 3282249-3282348 |
| 3158169 | 681 | TBK1 binding protein 1 | 0.203 | 3175184-3175283 |
| 3158197 | 284 | MHC, class I-related | 0.114 | 3163484-3163583 |
| 3158259 | 974 | histocompatibility 2, class II, locus Mb1 | 2.25 | 3256605-3256704 |
| 3158365 | 431 | complement component factor i | 0.209 | 3178584-3178683 |
| 3158381 | 762 | CD24a antigen | 0.906 | 3245917-3246016 |

### V. RNA EFFECTOR MODIFICATION

In some embodiments of the present invention, an oligonucleotide (e.g., a RNA effector molecule) is chemically modified to enhance stability or other beneficial characteristics. In one embodiment the RNA effector molecule is not chemically modified.

Oligonucleotides can be modified to prevent rapid degradation of the oligonucleotides by endo- and exo-nucleases and avoid undesirable off-target effects. The nucleic acids featured in the invention can be synthesized and/or modified by methods well established in the art, such as those described in CURRENT PROTOCOLS IN NUCLEIC ACID CHEMISTRY (Beaucage et al., eds., John Wiley & Sons, Inc., NY). Modifications include, for example, (a) end modifications, e.g., 5′ end modifications (phosphorylation, conjugation, inverted linkages, etc.), or 3′ end modifications (conjugation, DNA nucleotides, inverted linkages, etc.); (b) base modifications, e.g., replacement with stabilizing bases, destabilizing bases, or bases that base pair with an expanded repertoire of partners, removal of bases (abasic nucleotides), or conjugated bases; (c) sugar modifications (e.g., at the 2′ position or 4′ position) or replacement of the sugar; as well as (d) internucleoside linkage modifications, including modification or replacement of the phosphodiester linkages. Specific examples of oligonucleotide compounds useful in this invention include, but are not limited to RNAs containing modified backbones or no natural internucleoside linkages. RNAs having modified backbones include, among others, those that do not have a phosphorus atom in the backbone. Specific examples of oligonucleotide compounds useful in this invention include, but are not limited to oligonucleotides containing modified or non-natural internucleoside linkages. Oligonucleotides having modified internucleoside linkages include, among others, those that do not have a phosphorus atom in the internucleoside linkage.

For the purposes of this specification, and as sometimes referenced in the art, modified oligonucleotides that do not have a phosphorus atom in their internucleoside linkage(s) can also be considered to be oligonucleosides. In particular embodiments, the modified oligonucleotides will have a phosphorus atom in its internucleoside linkage(s). For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified internucleoside linkages include (e.g., RNA backbones) include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative patents that teach the preparation of the above phosphorus-containing linkages include, but are not limited to, U.S. Pat. No. 3,687,808; No. 4,469,863; No. 4,476,301; No. 5,023,243; No. 5,177,195; No. 5,188,897; No. 5,264,423; No. 5,276,019; No. 5,278,302; No. 5,286,717; No. 5,321,131; No. 5,399,676; No. 5,405,939; No. 5,453,496; No. 5,455,233; No. 5,466,677; No. 5,476,925; No. 5,519,126; No. 5,536,821; No. 5,541,316; No. 5,550,111; No. 5,563,253; No. 5,571,799; No. 5,587,361; No. 5,625,050; No. 6,028,188; No. 6,124,445; No. 6,160,109; No. 6,169,170; No. 6,172,209; No. 6,239,265; No. 6,277,603; No. 6,326,199; No. 6,346,614; No. 6,444,423; No. 6,531,590; No. 6,534,639; No. 6,608,035; No. 6,683,167; No. 6,858,715; No. 6,867,294; No. 6,878,805; No. 7,015,315; No. 7,041,816; No. 7,273,933; No. 7,321,029; and No. RE39464.

Modified oligonucleotide internucleoside linkages (e.g., RNA backbones) that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative patents that teach the preparation of the above oligonucleosides include, but are not limited to, U.S. Pat. No. 5,034,506; No. 5,166,315; No. 5,185,444; No. 5,214,134; No. 5,216,141; No. 5,235,033; No. 5,64,562; No. 5,264,564; No. 5,405,938; No. 5,434,257; No. 5,466,677; No. 5,470,967; No. 5,489,677; No. 5,541,307; No. 5,561,225; No. 5,596,086; No. 5,602,240; No. 5,608,046; No. 5,610,289; No. 5,618,704; No. 5,623,070; No. 5,663,312; No. 5,633,360; No. 5,677,437; and No. 5,677,439.

In other modified oligonucleotides suitable or contemplated for use in RNA effector molecules, both the sugar and the internucleoside linkage, i.e., the backbone, of the nucleotide units are replaced with novel groups. The base units are maintained for hybridization with an appropriate nucleic acid target compound. One such oligomeric compound, a RNA mimetic that has been shown to have excellent hybridization properties, is referred to as a peptide nucleic acid (PNA). In PNA compounds, the sugar backbone of a RNA is replaced with an amide containing backbone, in particular an aminoethylglycine backbone. The nucleobases are retained and are bound directly or indirectly to aza nitrogen atoms of the amide portion of the backbone. Representative patents that teach the preparation of PNA compounds include, but are not limited to, U.S. Pat. No. 5,539,082; No. 5,714,331; and No. 5,719,262. Further teaching of PNA compounds can be found, for example, in Nielsen et al., 254 Science 1497-1500 (1991).

Some embodiments featured in the invention include oligonucleotides with phosphorothioate internucleoside linkages and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester internucleoside linkage is represented as —O—P—O—CH₂—] (see U.S. Pat. No. 5,489,677), and amide backbones (see U.S. Pat. No. 5,602,240). In some embodiments, the oligonucleotides featured herein have morpholino backbone structures (see U.S. Pat. No. 5,034,506).

Modified oligonucleotides can also contain one or more substituted sugar moieties. The RNA effector molecules, e.g., dsRNAs, featured herein can include one of the following at the 2′ position: H (deoxyribose); OH (ribose); F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to 10, inclusive. In some embodiments, oligonucleotides include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a RNA cleaving group, a reporter group, an intercalator, a group for improving the pharmacokinetic properties of an oligonucleotide (e.g., a RNA effector molecule), or a group for improving the pharmacodynamic properties of an oligonucleotide (e.g., a RNA effector molecule), and other substituents having similar properties. In some embodiments, the modification includes a 2′-methoxyethoxy (2′-β-CH₂CH₂OCH₃, also known as 2′-O-(2-methoxyethyl) or 2′-MOE) (Martin et al., 78 Helv. Chim. Acta 486-504 (1995)), i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the oligonucleotide, particularly the 3′ position of the sugar on the 3′ terminal nucleotide or in 2′-5′ linked oligonucleotide and the 5′ position of 5′ terminal nucleotide. Oligonucleotides can also have sugar mimetics such as cyclobutyl moieties in place of the pentofuranosyl sugar. Representative patents that teach the preparation of such modified sugar structures include, but are not limited to, U.S. Pat. No. 4,981,957; No. 5,118,800; No. 5,319,080; No. 5,359,044; No. 5,393,878; No. 5,446,137; No. 5,466,786; No. 5,514,785; No. 5,519,134; No. 5,567,811; No. 5,576,427; No. 5,591,722; No. 5,597,909; No. 5,610,300; No. 5,627,053; No. 5,639,873; No. 5,646,265; No. 5,658,873; No. 5,670,633; and No. 5,700,920, certain of which are commonly owned with the instant application.

An oligonucleotide (e.g., a RNA effector molecule) can also include nucleobase (often referred to in the art simply as “base”) modifications or substitutions. As used herein, “unmodified” or “natural” nucleobases include the purine bases adenine (A) and guanine (G), and the pyrimidine bases cytosine (C) and uracil (U). Modified nucleobases include other synthetic and natural nucleobases such as as inosine, xanthine, hypoxanthine, nubularine, isoguanisine, tubercidine, 2-(halo)adenine, 2-(alkyl)adenine, 2-(propyl)adenine, 2 (amino)adenine, 2-(aminoalkyll)adenine, 2 (aminopropyl)adenine, 2 (methylthio) N6 (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl)adenine, 8-(thiol)adenine, N6-(isopentyl)adenine, N6 (methyl)adenine, N6, N6 (dimethyl)adenine, 2-(alkyl)guanine, 2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N (methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, (halo)cytosine, 5 (methyl)cytosine, 5 (propynyl)cytosine, 5 (propynyl)cytosine, (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, 5 (methyl) 2,4 (dithio)uracil, 5 (methylaminomethyl)-2,4 (dithio)uracil, 5 (2-aminopropyl)uracil, 5-(alkyl)uracil, 5-(alkynyl)uracil, 5-(allylamino)uracil, 5 (aminoallyl)uracil, (aminoalkyl)uracil, 5 (guanidiniumalkyl)uracil, 5 (1,3-diazole-1-alkyl)uracil, 5-(cyanoalkyl)uracil, 5-(dialkylaminoalkyl)uracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil, 4 (thio)pseudouraci-1,2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 (aminocarbonylethylenyl)-2,4-(dithio)pseudouracil, 1 (aminoalkylaminocarbonylethylenyl)-pseudouracil, 1 (aminoalkylamino-carbonylethylenyl)-2(thio)-pseudouracil, 1 (aminoalkylaminocarbonylethylenyl)-4 (thio)pseudouracil, 1 (aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenzyl, tetracenyl, pentacenyl, difluorotolyl, 4-(fluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino)purine, 5 substituted pyrimidines, N2-substituted purines, N6-substituted purines, 06-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Modified nucleobases also include natural bases that comprise conjugated moieties, e.g., a ligand.

Further nucleobases include those disclosed in U.S. Pat. No. 3,687,808; MODIFIED NUCLEOSIDES BIOCHEM., BIOTECH. & MEDICINE (Herdewijn, ed., Wiley-VCH, 2008); WO 2009/120878; CONCISE ENCYCLOPEDIA OF POLYMER SCIENCE & ENGIN. 858-59 (Kroschwitz ed., John Wiley & Sons, 1990); Englisch et al., 30 Angewandte Chemie, Intl. Ed. 613 (1991); Sanghvi, 15 DsRNA RESEARCH & APPLICATIONS 289-302 (Crooke & Lebleu, eds., CRC Press, Boca Raton, Fla., 1993). Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, at 276-78), and are exemplary base substitutions, even more particularly when combined with 2′-O-methoxyethyl sugar modifications.

Representative patents that teach the preparation of certain of the above noted modified nucleobases as well as other modified nucleobases include, but are not limited to, the above noted U.S. Pat. No. 3,687,808; No. 4,845,205; No. 5,130,30; No. 5,134,066; No. 5,175,273; No. 5,367,066; No. 5,432,272; No. 5,457,191No. 5,457,187; No. 5,459,255; No. 5,484,908; No. 5,502,177; No. 5,525,711; No. 5,552,540; No. 5,587,469; No. 5,594,121, No. 5,596,091; No. 5,614,617; No. 5,681,941; No. 6,015,886; No. 6,147,200; No. 6,166,197; No. 6,222,025; No. 6,235,887; No. 6,380,368; No. 6,528,640; No. 6,639,062; No. 6,617,438; No. 7,045,610; No. 7,427,672; and No. 7,495,088; and No. 5,750,692.

The oligonucleotides can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to oligonucleotide molecules has been shown to increase oligonucleotide molecule stability in serum, and to reduce off-target effects. Elmen et al., 33 Nucl. Acids Res. 439-47 (2005); Mook et al., 6 Mol. Cancer. Ther. 833-43 (2007); Grunweller et al., 31 Nucl. Acids Res. 3185-93 (2003); U.S. Pat. No. 6,268,490; No. 6,670,461; No. 6,794,499; No. 6,998,484; No. 7,053,207; No. 7,084,125; and No. 7,399,845.

In certain instances, the oligonucleotides of a RNA effector molecule can be modified by a non-ligand group. A number of non-ligand molecules have been conjugated to oligonucleotides in order to enhance the activity, cellular distribution or cellular uptake of the oligonucleotides, and procedures for performing such conjugations are available in the scientific literature. Such non-ligand moieties have included lipid moieties, such as cholesterol (Kubo et al., 365 Biochem. Biophys. Res. Comm. 54-61 (2007)); Letsinger et al., 86 PNAS 6553 (1989)); cholic acid (Manoharan et al., 1994); a thioether, e.g., hexyl-5-tritylthiol (Manoharan et al., 1992; Manoharan et al., 1993); a thiocholesterol (Oberhauser et al., 1992); an aliphatic chain, e.g., dodecandiol or undecyl residues (Saison-Behmoaras et al., 1991; Kabanov et al., 259 FEBS Lett. 327 (1990); Svinarchuk et al., 75 Biochimie 75 (1993)); a phospholipid, e.g., di-hexadecyl-rac-glycerol or triethylammonium 1,2-di-O-hexadecyl-rac-glycero-3-H-phosphonate (Manoharan et al., 1995); Shea et al., 18 Nucl. Acids Res. 3777 (1990)); a polyamine or a polyethylene glycol chain (Manoharan et al., Nucleosides & Nucleotides, 1995); or adamantane acetic acid (Manoharan et al., Tetrahedron Lett., 1995); a palmityl moiety (Mishra et al., 1995); or an octadecylamine or hexylamino-carbonyl-oxycholesterol moiety (Crooke et al., 1996). Representative United States patents that teach the preparation of such RNA conjugates have been listed herein. Typical conjugation protocols involve the synthesis of an oligonucleotide bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

Nucleic acid sequences of exemplary RNA effector molecules are represented below using standard nomenclature, and specifically the abbreviations of Table 17, as follows:

TABLE 17
Abbreviations of nucleotide monomers used
in nucleic acid sequence representation.

| Abbreviation | Nucleotide(s) |
|---|---|
| A | adenosine |
| C | cytidine |
| G | guanosine |
| T | thymidine |
| U | uridine |
| N | any nucleotide (G, A, C, T or U) |
| a | 2′-O-methyladenosine |
| c | 2′-O-methylcytidine |
| g | 2′-O-methylguanosine |
| u | 2′-O-methyluridine |
| dT | 2′-deoxythymidine |
| s | phosphorothioate linkage |

These monomers, when present in an oligonucleotide, are mutually linked by 5′-3′-phosphodiester bonds.

Ligands

Another modification of the oligonucleotides (e.g., of a RNA effector molecule) featured in the invention involves chemically linking to the oligonucleotide one or more ligands, moieties or conjugates that enhance the activity, cellular distribution or cellular uptake of the oligonucleotide. Such moieties include but are not limited to lipid moieties such as a cholesterol moiety (Letsinger et al., 86 PNAS 6553-56 (1989); cholic acid (Manoharan et al., 4 Biorg. Med. Chem. Let. 1053-60 (1994)); a thioether, e.g., beryl-5-tritylthiol (Manoharan et al., 660 Ann. NY Acad. Sci. 306309 (1992); Manoharan et al., 3 Biorg. Med. Chem. Let. 2765-70 (1993)); a thiocholesterol (Oberhauser et al., 20 Nucl. Acids Res. 533-38 (1992)); an aliphatic chain, e.g., dodecandiol or undecyl residues (Saison-Behmoaras et al., 10 EMBO J. 1111-18 (1991); Kabanov et al., 259 FEBS Lett. 327-30 (1990); Svinarchuk et al., 75 Biochimie 49-54 (1993)); a phospholipid, e.g., di-hexadecyl-rac-glycerol or triethyl-ammonium 1,2-di-O-hexadecyl-rac-glycero-3-phosphonate (Manoharan et al., 36 Tetrahedron Lett. 3651-54 (1995); Shea et al., 18 Nucl. Acids Res. 3777-83 (1990)); a polyamine or a polyethylene glycol chain (Manoharan et al., 14 Nucleosides & Nucleotides 969-73 (1995)); or adamantane acetic acid (Manoharan et al., Tetrahedron Lett., 1995); a palmityl moiety (Mishra et al., 1264 Biochim. Biophys. Acta 229-37 (1995)); or an octadecylamine or hexylamino-carbonyloxycholesterol moiety (Crooke et al., 227 J. Pharmacol. Exp. Ther. 923-37 (1996)).

In one embodiment, a ligand alters the distribution, targeting or lifetime of a RNA effector molecule agent into which it is incorporated. In some embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Ideally, ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example polyamines include polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an -helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic molecules, e.g, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu³⁺ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the RNA effector molecule agent into the cell, for example, by disrupting the cell's cytoskeleton, e.g., by disrupting the cell's microtubules, microfilaments, and/or intermediate filaments. The drug can be, for example, taxol, vincristine, vinblastine, cytochalasin, nocodazole, japlakinolide, latrunculin A, phalloidin, swinholide A, indanocine, or myoservin.

An example ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, Naproxen or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the embryo. A lipid or lipid-based ligand that binds to HSA less strongly can be used to target the conjugate to the kidney. For example, the lipid based ligand binds HSA, or it binds HSA with a sufficient affinity such that the conjugate will be distributed to a non-kidney tissue but also be reversible. Alternatively, the lipid-based ligand binds HSA weakly or not at all, such that the conjugate will be distributed to the kidney. Other moieties that target to kidney cells can also be used in place of or in addition to the lipid-based ligand.

In another aspect, the ligand is a moiety, e.g., a vitamin, that is taken up by an embryonic cell, e.g., a proliferating cell. Exemplary vitamins include vitamin A, E, and K. Other exemplary vitamins include are B vitamin, e.g., folic acid, B12, riboflavin, biotin, pyridoxal or other vitamins or nutrients taken up by embryonic cells. Also included are HSA and low density lipoproteins.

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent can be an α-helical agent, and can include a lipophilic and a lipophobic phase.

The ligand can be a peptide or peptidomimetic. A peptidomimetic (also referred to herein as an oligopeptidomimetic) is a molecule capable of folding into a defined 3-dimensional structure similar to a natural peptide. The attachment of peptide and peptidomimetics to RNA effector molecule agents can affect pharmacokinetic distribution of the RNA effector molecule, such as by enhancing cellular recognition and absorption. The peptide or peptidomimetic moiety can be about 5 to 50 amino acids long, e.g., about 5, 10, 15, 20, 25, 30, 35, 40, 45, or 50 amino acids long (see Table 18, for example).

TABLE 18
Exemplary Cell Permeation Peptides

| Cell Permeation | | SEQ ID | |
|---|---|---|---|
| Peptide | Amino acid Sequence | NO: | Reference |
| Penetratin | RQIKIWFQNRRMKWKK | 3284943 | Derossi et al., 269 J. |
| | | | Biol. Chem. 10444 |
| | | | (1994) |
| Tat fragment | GRKKRRQRRRPPQC | 3284944 | Vives et al., 272 J. |
| (48-60) | | | Biol. Chem. 16010 |
| | | | (1997) |
| Signal | GALFLGWLGAAGSTMGAWSQPKKKRKV | 3284945 | Chaloin et al., 243 |
| Sequence-based | | | Biochem. Biophys. |
| peptide | | | Res. Commun 601 |
| | | | (1998) |
| PVEC | LLIILRRRIRKQAHAHSK | 3284946 | Elmquist et al., 269 |
| | | | Exp. Cell Res. 237 |
| | | | (2001) |
| Transportan | GWTLNSAGYLLKINLKALAALAKKIL | 3284947 | Pooga et al., 12 |
| | | | FASEB J. 67 (1998) |
| Amphiphilic | KLALKLALKALKAALKLA | 3284948 | Oehlke et al., 2 Mol. |
| model peptide | | | Ther. 339 (2000) |
| Arg₉ | RRRRRRRRR | 3284949 | Mitchell et al., 56 J. |
| | | | Pept. Res. 318 (2000) |
| Bacterial cell | KFFKFFKFFK | 3284950 | |
| wall permeating | | | |
| LL-37 | LLGDFFRKSKEKIGKEFKRIVQRIKDFLRN | 3284951 | |
| | LVPRTES | | |
| Cecropin P1 | SWLSKTAKKLENSAKKRISEGIAIAIQGGP | 3284952 | |
| | R | | |
| α-defensin | ACYCRIPACIAGERRYGTCIYQGRLWAFC | 3284953 | |
| | C | | |
| b-defensin | DHYNCVSSGGQCLYSACPIFTKIQGTCYR | 3284954 | |
| | GKAKCCK | | |
| Bactenecin | RKCRIVVIRVCR | 3284955 | |
| PR-39 | RRRPRPPYLPRPRPPPFFPPRLPPRIPPGFPP | 3284956 | |
| | RFPPRFPGKR-NH₂ | | |
| Indolicidin | ILPWKWPWWPWRR-NH₂ | 3284957 | |

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO:3284958) An RFGF analogue (e.g., amino acid sequence AALLPVLLAAP (SEQ ID NO:3284959) containing a hydrophobic MTS can also be a targeting moiety. The peptide moiety can be a “delivery” peptide that carres large polar molecules including peptides, oligonucleotides, and protein across cell membranes. For example, sequences from the HIV Tat protein (GRKKRRQRRRPPQ [SEQ ID NO:3284960]) and the Drosophila antennapedia protein (RQIKIWFQNRRMKWKK [SEQ ID NO:284961]) can function as delivery peptides. A peptide or peptidomimetic can be encoded by a random sequence of DNA, such as a peptide identified from a phage-display library, or one-bead-one-compound (OBOC) combinatorial library. Lam et al., 354 Nature 82-84 (1991). The peptide or peptidomimetic can be tethered to a dsRNA agent via an incorporated monomer unit is a cell targeting peptide such as an arginine-glycine-aspartic acid (RGD)-peptide, or RGD mimic. As noted, the peptide moieties can have a structural modification, such as to increase stability or direct conformational properties. Any of the structural modifications described herein can be utilized.

An RGD peptide moiety can be used to target a tumor cell, such as an endothelial tumor cell or a breast cancer tumor cell. Zitzmann et al., 62 Cancer Res. 5139-43 (2002). An RGD peptide can facilitate targeting of an dsRNA agent to tumors of a variety of other tissues, including the lung, kidney, spleen, or liver. Aoki et al., 8 Cancer Gene Ther. 783-87 (2001). Preferably, the RGD peptide will facilitate targeting of a RNA effector molecule agent to the kidney. The RGD peptide can be linear or cyclic, and can be modified, e.g., glycosylated or methylated to facilitate targeting to specific tissues. For example, a glycosylated RGD peptide can deliver a RNA effector molecule agent to a tumor cell expressing αVβ3. Haubner et al., 42 J. Nucl. Med. 326-36 (2001).

A “cell permeation peptide” is capable of permeating a cell. It can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen. Simeoni et al., 31 Nucl. Acids Res. 2717-24 (2003).

Representative patents that teach the preparation of oligonucleotide conjugates include, but are not limited to, U.S. Pat. No. 4,828,979; No. 4,948,882; No. 5,218,105; No. 5,525,465; No. 5,541,313; No. 5,545,730; No. 5,552,538; No. 5,578,717, No. 5,580,731; No. 5,591,584; No. 5,109,124; No. 5,118,802; No. 5,138,045; No. 5,414,077; No. 5,486,603; No. 5,512,439; No. 5,578,718; No. 5,608,046; No. 4,587,044; No. 4,605,735; No. 4,667,025; No. 4,762,779; No. 4,789,737; No. 4,824,941; No. 4,835,263; No. 4,876,335; No. 4,904,582; No. 4,958,013; No. 5,082,830; No. 5,112,963; No. 5,214,136; No. 5,082,830; No. 5,112,963; No. 5,214,136; No. 5,245,022; No. 5,254,469; No. 5,258,506; No. 5,262,536; No. 5,272,250; No. 5,292,873; No. 5,317,098; No. 5,371,241, No. 5,391,723; No. 5,416,203, No. 5,451,463; No. 5,510,475; No. 5,512,667; No. 5,514,785; No. 5,565,552; No. 5,567,810; No. 5,574,142; No. 5,585,481; No. 5,587,371; No. 5,595,726; No. 5,597,696; No. 5,599,923; No. 5,599,928; No. 5,688,941; No. 6,294,664; No. 6,320,017; No. 6,576,752; No. 6,783,931; No. 6,900,297; and No. 7,037,646.

It is not necessary for all positions in a given compound to be uniformly modified, and in fact more than one of the aforementioned modifications can be incorporated in a single compound or even at a single nucleoside within an oligonucleotide. The present invention also includes oligonucleotide molecule compounds which are chimeric compounds. “Chimeric” RNA effector molecule compounds or “chimeras,” in the context of this invention, are oligonucleotide compounds, such as dsRNAs, that contain two or more chemically distinct regions, each made up of at least one monomer unit, i.e., a nucleotide in the case of a dsRNA compound. These RNA effector molecules typically contain at least one region wherein the RNA is modified so as to confer upon the RNA effector molecule increased resistance to nuclease degradation, increased cellular uptake, and/or increased binding affinity for the target nucleic acid. An additional region of the oligonucleotide can serve as a substrate for enzymes capable of cleaving RNA:DNA or RNA:RNA hybrids. By way of example, RNase H is a cellular endonuclease which cleaves the RNA strand of a RNA:DNA duplex. Activation of RNase H, therefore, results in cleavage of the RNA target, thereby greatly enhancing the efficiency of RNA effector molecule inhibition of gene expression. Consequently, comparable results can often be obtained with shorter RNA effector molecules when chimeric dsRNAs are used, compared to phosphorothioate deoxydsRNAs hybridizing to the same target region. Cleavage of the oligonucleotide can be routinely detected by gel electrophoresis and, if necessary, associated nucleic acid hybridization techniques known in the art.

### VI. INTRODUCTION/DELIVERY OF RNA EFFECTOR MOLECULES

The delivery of an oligonucleotide (e.g., a RNA effector molecule) to cells according to methods provided herein can be achieved in a number of different ways. For example, delivery can be performed directly by administering a composition comprising a RNA effector molecule, e.g., a dsRNA, into cell culture. Alternatively, delivery can be performed indirectly by administering into the cell one or more vectors that encode and direct the expression of the RNA effector molecule. These alternatives are discussed further herein.

In some embodiments, the RNA effector molecule is a siRNA or shRNA effector molecule introduced into a cell by introducing into the cell an invasive bacterium containing one or more siRNA or shRNA effector molecules or DNA encoding one or more siRNA or shRNA effector molecules (a process sometimes referred to as transkingdom RNAi (tkRNAi)). The invasive bacterium can be an attenuated strain of Listeria, Shigella, Salmonella, E. coli, or Bifidobacteriae, or a non-invasive bacterium that has been genetically modified to increase its invasive properties, e.g., by introducing one or more genes that enable invasive bacteria to access the cytoplasm of cells. Examples of such cytoplasm-targeting genes include listeriolysin 0 of Listeria and the invasin protein of Yersinia pseudotuberculosis. Methods for delivering RNA effector molecules to animal cells to induce transkingdom RNAi (tkRNAi) are known in the art. See, e.g., U.S. Patent Pubs. No. 2008/0311081 and No. 2009/0123426. In one embodiment, the RNA effector molecule is a siRNA molecule. In one embodiment, the RNA effector molecule is not a shRNA molecule.

As noted herein, oligonucleotides can be modified to prevent rapid degradation of the dsRNA by endo- and exo-nucleases and avoid undesirable off-target effects. For example, RNA effector molecules can be modified by chemical conjugation to lipophilic groups such as cholesterol to enhance cellular uptake and prevent degradation. In one embodiment, the RNA effector molecule is not modified by chemical conjugation to a lipophilic group, e.g., cholesterol.

In an alternative embodiment, RNA effector molecules can be delivered using a drug delivery system such as a nanoparticle, a dendrimer, a polymer, a liposome, or a cationic delivery system. Positively charged cationic delivery systems facilitate binding of a RNA effector molecule (negatively charged) and also enhance interactions at the negatively charged cell membrane to permit efficient cellular uptake. Cationic lipids, dendrimers, or polymers can either be bound to RNA effector molecules, or induced to form a vesicle or micelle that encases the RNA effector molecule. See, e.g., Kim et al., 129 J. Contr. Release 107-16 (2008). Methods for making and using cationic-RNA effector molecule complexes are well within the abilities of those skilled in the art. See e.g., Sorensen et al 327 J. Mol. Biol. 761-66 (2003); Verma et al., 9 Clin. Cancer Res. 1291-1300 (2003); Arnold et al., 25 J. Hypertens. 197-205 (2007).

Where the RNA effector molecule is a double-stranded molecule, such as a small interfering RNA (siRNA), comprising a sense strand and an antisense strand, the sense strand and antisense strand can be separately and temporally exposed to a cell, cell lysates, tissue, or cell culture. The phrase “separately and temporally” refers to the introduction of each strand of a double-stranded RNA effector molecule to a cell, cell lysates, tissue or cell culture in a single-stranded form, e.g., in the form of a non-annealed mixture of both strands or as separate, i.e., unmixed, preparations of each strand. In some embodiments, there is a time interval between the introduction of each strand which can range from seconds to several minutes to about an hour or more, e.g., 12 hr, 24 hr, 48 hr, 72 hr, 84 hr, 96 hr, or 108 hr, or more. Separate and temporal administration can be performed with canonical or non-canonical RNA effector molecules.

It is also contemplated herein that a plurality of RNA effector molecules are administered in a separate and temporal manner. Thus, each of a plurality of RNA effector molecules can be administered at a separate time or at a different frequency interval to achieve the desired average percent inhibition for the target gene. For example, RNA effector molecules targeting Bak can be administered more frequently than RNA effector molecule targeting LDH, as the expression of Bak recovers faster following treatment with a Bak RNA effector molecule. In one embodiment, the RNA effector molecules are added at a concentration from approximately 0.01 nM to 200 nM. In another embodiment, the RNA effector molecules are added at an amount of approximately 50 molecules per cell up to and including 500,000 molecules per cell. In another embodiment, the RNA effector molecules are added at a concentration from about 0.1 fmol/10⁶ cells to about 1 pmol/10⁶ cells.

In another aspect, a RNA effector molecule for modulating expression of a target gene can be expressed from transcription units inserted into DNA or RNA vectors. See, e.g., Couture et al., 12 TIG 5-10 (1996); WO 00/22113; WO 00/22114; U.S. Pat. No. 6,054,299. Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extra chromosomal plasmid. Gassmann, et al., 92 PNAS 1292 (1995).

The individual strand or strands of a RNA effector molecule can be transcribed from a promoter on an expression vector. Where two separate strands are to be expressed to generate, for example, a dsRNA, two separate expression vectors can be co-introduced (e.g., by transfection or infection) into a target cell. Alternatively each individual strand of a dsRNA can be transcribed by promoters both of which are located on the same expression plasmid. In one embodiment, a dsRNA is expressed as an inverted repeat joined by a linker polynucleotide sequence such that the dsRNA has a stem and loop structure.

RNA effector molecule expression vectors are generally DNA plasmids or viral vectors. Expression vectors compatible with eukaryotic cells, such as those compatible with vertebrate cells, insect cells, or yeast cells can be used to produce recombinant constructs for the expression of a RNA effector molecule as described herein. Eukaryotic cell expression vectors are well known in the art and are available from a number of commercial sources. Typically, such vectors are provided containing convenient restriction sites for insertion of the desired nucleic acid segment. RNA effector molecule expressing vectors can be delivered directly to target cells using standard transfection and transduction methods.

RNA effector molecule expression plasmids can be transfected into target cells as a complex with cationic lipid carriers (e.g., OLIGOFECTAMINE™ transfection reagent) or non-cationic lipid-based carriers (e.g., TRANSIT-TKO® transfection reagent, Minis Bio LLC, Madison, Wis.). Multiple lipid transfections for RNA effector molecule-mediated knockdowns targeting different regions of a target RNA over a period of a week or more are also contemplated by the invention. Successful introduction of vectors into host cells can be monitored using various known methods. For example, transient transfection can be signaled with a reporter, such as a fluorescent marker, such as Green Fluorescent Protein (GFP). Stable transfection of cells ex vivo can be ensured using markers that provide the transfected cell with resistance to specific environmental factors (e.g., antibiotics and drugs), such as hygromycin B resistance. RNA effector molecule expression plasmids can be transfected into target cells as a complex with cationic lipid carriers (e.g., OLIGOFECTAMINE™ reagent) or non-cationic lipid-based carriers (e.g., TRANSIT-TKO® transfection reagent). Multiple lipid transfections for RNA effector molecule-mediated knockdowns targeting different regions of a target RNA over a period of a week or more are also contemplated by the invention. Successful introduction of vectors into host cells can be monitored using various known methods. For example, transient transfection can be signaled with a reporter, such as a fluorescent marker, such as GFP. Stable transfection of cells ex vivo can be ensured using markers that provide the transfected cell with resistance to specific environmental factors (e.g., antibiotics and drugs), such as hygromycin B resistance.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g., EPV and EBV vectors. Constructs for the recombinant expression of a RNA effector molecule will generally require regulatory elements, e.g., promoters, enhancers, etc., to ensure the expression of the RNA effector molecule in target cells. Other aspects to consider for vectors and constructs are further described herein.

Vectors useful for the delivery of a RNA effector molecule will include regulatory elements (promoter, enhancer, etc.) sufficient for expression of the RNA effector molecule in the desired target cell or tissue. The regulatory elements can be chosen to provide either constitutive or regulated/inducible expression.

Expression of the RNA effector molecule can be precisely regulated, for example, by using an inducible regulatory sequence that is sensitive to certain physiological regulators, e.g., glucose levels. Docherty et al., 8 FASEB J. 20-24 (1994). Such inducible expression systems, suitable for the control of dsRNA expression in cells include, for example, regulation by ecdysone, estrogen, progesterone, tetracycline, chemical inducers of dimerization, and isopropyl-β-D1-thiogalactopyranoside (IPTG). A person skilled in the art would be able to choose the appropriate regulatory/promoter sequence based on the intended use of the RNA effector molecule transgene.

In a specific embodiment, viral vectors that contain nucleic acid sequences encoding a RNA effector molecule can be used. For example, a retroviral vector can be used. See Miller et al., 217 Meth. Enzymol. 581-99 (1993); U.S. Pat. No. 6,949,242. Retroviral vectors contain the components necessary for the correct packaging of the viral genome and integration into the host cell DNA. The nucleic acid sequences encoding a RNA effector molecule are cloned into one or more vectors, which facilitates delivery of the nucleic acid into a cell. More detail about retroviral vectors can be found, for example, in Boesen et al., 6 Biotherapy 291-302 (1994), which describes the use of a retroviral vector to deliver the mdr1 gene to hematopoietic stem cells in order to make the stem cells more resistant to chemotherapy. Other references illustrating the use of retroviral vectors in gene therapy include Clowes et al., 93 J. Clin. Invest. 644-651 (1994); Kiem et al., 83 Blood 1467-73 (1994); Salmons & Gunzberg, 4 Human Gene Ther. 129-11 (1993); Grossman & Wilson, 3 Curr. Opin. Genetics Devel. 110-14 (1993). Lentiviral vectors contemplated for use include, for example, the HIV based vectors described in U.S. Pat. No. 6,143,520; No. 5,665,557; and No. 5,981,276.

It should be noted, as discussed herein, that host cell-surface receptors for retroviral entry can be inhabited by ERV Env proteins (virus interference). See Miller, 93 PNAS 11407-13 (1996). The retroviral envelope (Env) protein mediates the binding of virus particles to their cellular receptors, enabling virus entry: the first step in a new replication cycle. If an ERV is expressed in a cell, re-infection by a related exogenous retrovirus is prevented through interference (also called receptor interference): the Env protein of an ERV that is inserted into the cell membrane will interfere with the corresponding exogenous virus by receptor competition. This protects the cell from being overloaded with retroviruses. For example, enJSRVs can block the entry of exogenous JSRVs because they all utilize the cellular hyaluronidase-2 as a receptor. Spencer et al., 77 J. Virol. 5749-53 (2003). It is noteworthy that defective ERVs are no less interfering. Two enJSRVs, enJS56A1 and enJSRV-20, contain a mutant Gag polyprotein that can interfere with the late stage replication of exogenous JSRVs. Arnaud et al., 2 PLoS e170 (2007). Thus, interference between defective and replication-competent retroviruses provides an important mechanism of ERV copy number control. Receptor interference by ERV-expressed Env molecules (e.g., expressed by the HERV-H family) can hinder transfection or re-infection of cells intended to produce recombinant proteins. Such effects can explain low copy number or low expression in retroviral vector-mediated recombinant host cells, such as host cells transfected with two retroviral vectors, each encoding a single, complementary antibody chain. Hence, a target gene of the present embodiments that inhibits expression of ERV Env protein(s) provides for increasing retroviral vector multiplicity in host cells and increased yield of immunogenic agent.

Adenoviruses are also contemplated for use in delivery of RNA effector molecules. A suitable AV vector for expressing a RNA effector molecule featured in the invention, a method for constructing the recombinant AV vector, and a method for delivering the vector into target cells, are described in Xia et al., 20 Nat. Biotech. 1006-10 (2002).

Use of Adeno-associated virus (AAV) vectors is also contemplated (Walsh et al., 204 Proc. Soc. Exp. Biol. Med. 289-300 (1993); U.S. Pat. No. 5,436,146. In one embodiment, the RNA effector molecule can be expressed as two separate, complementary single-stranded RNA molecules from a recombinant AAV vector having, for example, either the U6 or H1 RNA promoters, or the cytomegalovirus (CMV) promoter. Suitable AAV vectors for expressing the dsRNA featured in the invention, methods for constructing the recombinant AV vector, and methods for delivering the vectors into target cells are described in Samulski et al., 61 J. Virol. 3096-101 (1987); Fisher et al., 70 J. Virol, 70: 520-32 (1996); Samulski et al., 63 J. Virol. 3822-26 (1989); U.S. Pat. No. 5,252,479 and No. 5,139,941; WO 94/13788; WO 93/24641.

Another viral vector is a pox virus such as a vaccinia virus, for example an attenuated vaccinia such as Modified Virus Ankara (MVA) or NYVAC, an avipox such as fowl pox or canary pox.

The tropism of viral vectors can be modified by pseudotyping the vectors with envelope proteins or other surface antigens from other viruses, or by substituting different viral capsid proteins, as appropriate. For example, lentiviral vectors can be pseudotyped with surface proteins from vesicular stomatitis virus (VSV), rabies, Ebola, Mokola, Baculovirus, and the like. Mononegavirales, e.g., VSV or respiratory syncytial virus (RSV) can be pseudotyped with Baculovirus. U.S. Pat. No. 7,041,489. AAV vectors can be made to target different cells by engineering the vectors to express different capsid protein serotypes. See, e.g., Rabinowitz et al., 76 J. Virol. 791-801 (2002).

In one embodiment, the invention provides compositions containing a RNA effector molecule, as described herein, and an acceptable carrier. The composition containing the RNA effector molecule is useful for enhancing the production of an immunogenic agent by a cell by modulating the expression or activity of a target gene in the cell. Such compositions are formulated based on the mode of delivery. Provided herein are exemplary RNA effector molecules useful in improving the production of an immunogenic agent. In one embodiment, the RNA effector molecule in the composition is a siRNA. Alternatively, the RNA effector molecule in the composition is not a siRNA.

In another embodiment, a composition is provided herein comprising a plurality of RNA effector molecules that permit inhibition of expression of an immune response pathway and a cellular process; such as INFRA1 or IFNB genes, and PTEN, BAK, FN1 or LDHA genes. The composition can optionally be combined (or administered) with at least one additional RNA effector molecule targeting an additional cellular process including, but not limited to: carbon metabolism and transport, apoptosis, RNAi uptake and/or efficiency, reactive oxygen species production, cell cycle control, protein folding, pyroglutamation protein modification, deamidase, glycosylation, disulfide bond formation, protein secretion, gene amplification, viral replication, viral infection, viral particle release, control of pH, and protein production.

In one embodiment, the compositions described herein comprise a plurality of RNA effector molecules. In one embodiment of this aspect, each of the plurality of RNA effector molecules is provided at a different concentration. In another embodiment of this aspect, each of the plurality of RNA effector molecules is provided at the same concentration. In another embodiment of this aspect, at least two of the plurality of RNA effector molecules are provided at the same concentration, while at least one other RNA effector molecule in the plurality is provided at a different concentration. It is appreciated one of skill in the art that a variety of combinations of RNA effector molecules and concentrations can be provided to a cell in culture to produce the desired effects described herein.

In one embodiment, a first RNA effector molecule is administered to a cultured cell, and then a second RNA effector molecule is administered to the cell (or vice versa). In a further embodiment, the first and second RNA effector molecules are administered to a cultured cell substantially simultaneously.

In another embodiment, a composition containing a RNA effector molecule described herein, e.g., a dsRNA directed against a host cell target gene, is administered to a cultured cell with a non-RNA agent useful for enhancing the production of an immunogenic by the cell.

The compositions featured herein are administered in amounts sufficient to inhibit expression of target genes. In general, a suitable dose of RNA effector molecule will be in the range of 0.001 to 200.0 milligrams per unit volume per day. In another embodiment, the RNA effector molecule is provided in the range of 0.001 nM to 200 mM per day, generally in the range of 0.1 nM to 500 nM, inclusive. For example, the dsRNA can be administered at 0.01 nM, 0.05 nM, 0.1 nM, 0.5 nM, 0.75 nM, 1 nM, 1.5 nM, 2 nM, 3 nM, 10 nM, 20 nM, 30 nM, 40 nM, 50 nM, 100 nM, 200 nM, 400 nM, or 500 nM per single dose.

The composition can be administered once daily, or the RNA effector molecule can be administered as two, three, or more sub-doses at appropriate intervals throughout the day or delivery through a controlled release formulation. In that case, the RNA effector molecule contained in each sub-dose must be correspondingly smaller in order to achieve the total daily dosage. The dosage unit can also be compounded for delivery over several days, e.g., using a conventional sustained release formulation, which provides sustained release of the RNA effector molecule over a several-day-period. Sustained release formulations are well known in the art and are particularly useful for delivery of agents to a particular site, such as could be used with the agents of the present invention. It should be noted that when administering a plurality of RNA effector molecules, one should consider that the total dose of RNA effector molecules will be higher than when each is administered alone. For example, administration of three RNA effector molecules each at 1 nM (e.g., for effective inhibition of target gene expression) will necessarily result in a total dose of 3 nM to the cell. One of skill in the art can modify the necessary amount of each RNA effector molecule to produce effective inhibition of each target gene while preventing any unwanted toxic effects to the embryo resulting from high concentrations of either the RNA effector molecules or delivery agent.

The effect of a single dose on target gene transcript levels can be long-lasting, such that subsequent doses are administered at not more than 3-, 4-, or 5-day intervals, or at not more than 1-, 2-, 3-, or 4-week intervals.

In one embodiment, the administration of the RNA effector molecule is ceased at least 6 hr, at least 12 hr, at least 18 hr, at least 36 hr, at least 48 hr, at least 60 hr, at least 72 hr, at least 96 hr, or at least 120 hr, or at least 1 week, before isolation of the immunogenic agent. Thus in one embodiment, contacting a host cell (e.g. in a large scale host cell culture) with a RNA effector molecule is complete at least 6 hr, at least 12 hr, at least 18 hr, at least 36 hr, at least 48 hr, at least 60 hr, at least 72 hr, at least 96 hr, or at least 120 hr, or at least 1 week, before isolation of the immunogenic agent.

It is also noted that, in certain embodiments, it can be beneficial to contact the cells in culture with a RNA effector molecule such that a constant number (or at least a minimum number) of RNA effector molecules per each cell is maintained. Maintaining the levels of the RNA effector molecule as such can ensure that modulation of target gene expression is maintained even at high cell densities.

Alternatively, the amount of a RNA effector molecule can be administered according to the cell density. In such embodiments, the RNA effector molecule(s) is added at a concentration of at least 0.01 fmol/10⁶ cells, at least 0.1 fmol/10⁶ cells, at least 0.5 fmol/10⁶ cells, at least 0.75 fmol/10⁶ cells, at least 1 fmol/10⁶ cells, at least 2 fmol/10⁶ cells, at least 5 fmol/10⁶ cells, at least 10 fmol/10⁶ cells, at least 20 fmol/10⁶ cells, at least 30 fmol/10⁶ cells, at least 40 fmol/10⁶ cells, at least 50 fmol/10⁶ cells, at least 60 fmol/10⁶ cells, at least 100 fmol/10⁶ cells, at least 200 fmol/10⁶ cells, at least 300 fmol/10⁶ cells, at least 400 fmol/10⁶ cells, at least 500 fmol/10⁶ cells, at least 700 fmol/10⁶ cells, at least 800 fmol/10⁶ cells, at least 900 fmol/10⁶ cells, or at least 1 pmol/10⁶ cells, or more.

In an alternate embodiment, the RNA effector molecule is administered at a dose of at least 10 molecules per cell, at least 20 molecules per cell (molecules/cell), at least 30 molecules/cell, at least 40 molecules/cell, at least 50 molecules/cell, at least 60 molecules/cell, at least 70 molecules/cell, at least 80 molecules/cell, at least 90 molecules/cell at least 100 molecules/cell, at least 200 molecules/cell, at least 300 molecules/cell, at least 400 molecules/cell, at least 500 molecules/cell, at least 600 molecules/cell, at least 700 molecules/cell, at least 800 molecules/cell, at least 900 molecules/cell, at least 1000 molecules/cell, at least 2000 molecules/cell, at least 5000 molecules/cell or more, inclusive.

In some embodiments, the RNA effector molecule is administered at a dose within the range of 10-100 molecules/cell, 10-90 molecules/cell, 10-80 molecules/cell, 10-70 molecules/cell, 10-60 molecules/cell, 10-50 molecules/cell, 10-40 molecules/cell, 10-30 molecules/cell, 10-20 molecules/cell, 90-100 molecules/cell, 80-100 molecules/cell, 70-100 molecules/cell, 60-100 molecules/cell, 50-100 molecules/cell, 40-100 molecules/cell, 30-100 molecules/cell, 20-100 molecules/cell, 30-60 molecules/cell, 30-50 molecules/cell, 40-50 molecules/cell, 40-60 molecules/cell, or any range there between.

In one embodiment of the methods described herein, the RNA effector molecule is provided to the cells in a continuous infusion. The continuous infusion can be initiated at day zero (e.g., the first day of cell culture or day of inoculation with a RNA effector molecule) or can be initiated at any time period during the immunogen production process. Similarly, the continuous infusion can be stopped at any time point during the immunogenic agent production process. Thus, the infusion of a RNA effector molecule or composition can be provided and/or removed at a particular phase of cell growth, a window of time in the production process, or at any other desired time point. The continuous infusion can also be provided to achieve a “desired average percent inhibition” for a target gene, as that term is used herein.

In one embodiment, a continuous infusion can be used following an initial bolus administration of a RNA effector molecule to a cell culture. In this embodiment, the continuous infusion maintains the concentration of RNA effector molecule above a minimum level over a desired period of time. The continuous infusion can be delivered at a rate of 0.03 pmol/L of culture/hour to 3 pmol/L of culture/hour, for example, at 0.03 pmol/L/hr, 0.05 pmol/L/hr, 0.08 pmol/L/hr, 0.1 pmol/L/hr, 0.2 pmol/L/hr, 0.3 pmol/L/hr, 0.5 pmol/L/hr, 1.0 pmol/L/hr, 2 pmol/L/hr, or 3 pmol/L/hr, or any value there between.

In one embodiment, the RNA effector molecule is administered as a sterile aqueous solution. In one embodiment, the RNA effector molecule is formulated in a non-lipid formulation. In another embodiment, the RNA effector molecule is formulated in a cationic or non-cationic lipid formulation. In still another embodiment, the RNA effector molecule is formulated in a cell medium suitable for culturing a host cell (e.g., a serum-free medium). In one embodiment, an initial concentration of RNA effector molecule(s) is supplemented with a continuous infusion of the RNA effector molecule to maintain modulation of expression of a target gene. In another embodiment, the RNA effector molecule is applied to cells in culture at a particular stage of cell growth (e.g., early log phase) in a bolus dosage to achieve a certain concentration (e.g., 1 nM), and provided with a continuous infusion of the RNA effector molecule.

The RNA effector molecule(s) can be administered once daily, or the RNA effector molecule treatment can be repeated (e.g., two, three, or more doses) by adding the composition to the culture medium at appropriate intervals/frequencies throughout the production of the immunogenic agent. As used herein the term “frequency” refers to the interval at which transfection of the cell culture occurs and can be optimized by one of skill in the art to maintain the desired level of inhibition for each target gene. In one embodiment, RNA effector molecules are contacted with cells in culture at a frequency of every 48 hours. In other embodiments, the RNA effector molecules are administered at a frequency of e.g., every 4 hr, every 6 hr, every 12 hr, every 18 hr, every 24 hr, every 36 hr, every 72 hr, every 84 hr, every 96 hr, every 5 days, every 7 days, every 10 days, every 14 days, every 3 weeks, or more during the production of the immunogenic agent. The frequency can also vary, such that the interval between each dose is different (e.g., first interval 36 hr; second interval 48 hr; third interval 72 hr, etc).

The term “frequency” can be similarly applied to nutrient feeding of a cell culture during the production of an immunogenic agent. The frequency of treatment with RNA effector molecule(s) and nutrient feeding need not be the same. To be clear, nutrients can be added at the time of RNA effector treatment or at an alternate time. The frequency of nutrient feeding can be a shorter interval or a longer interval tha RNA effector molecule treatment. For example, the dose of RNA effector molecule can be applied at a 48-hour-interval while nutrient feeding can be applied at a 24-hour-interval. During the entire length of the interval for producing the immunogenic product (e.g., 3 weeks) there can be more doses of nutrients than RNA effector molecules or less doses of nutrients than RNA effector molecules. Alternatively, the amount of treatments with RNA effector molecule(s) is equal to that of nutrient feedings.

The frequency of RNA effector molecule treatment can be optimized to maintain an “average percent inhibition” of a particular target gene. As used herein, the term “desired average percent inhibition” refers to the average degree of inhibition of target gene expression over time that is necessary to produce the desired effect and which is below the degree of inhibition that produces any unwanted or negative effects. For example, the desired inhibition of Bax/Bak is typically >80% inhibition to effect a decrease in apoptosis, while the desired average inhibition of LDH can be less (e.g., 70%) as high degrees of LDH average inhibition (e.g., 90%) decrease cell viability. In some embodiments, the desired average percent inhibition is at least 20%, at least 30%, at least 40%, at least 50%, at least 60%, at least 70%, at least 80%, at least 85%, at least 90%, at least 95%, at least 99%, or even 100% (i.e., absent). One of skill in the art can use routine cell death assays to determine the upper limit for desired percent inhibition (e.g., level of inhibition that produces unwanted effects). One of skill in the art can also use methods to detect target gene expression (e.g., PERT) to determine an amount of a RNA effector molecule that produces gene modulation. See Zhang et al., 102 Biotech. Bioeng. 1438-47 (2009). The percent inhibition is described herein as an average value over time, since the amount of inhibition is dynamic and can fluctuate slightly between doses of the RNA effector molecule.

In one embodiment of the methods described herein, the RNA effector molecule is added to the culture medium of the cells in culture. The methods described herein can be applied to any size of cell culture flask and/or bioreactor. For example, the methods can be applied in bioreactors or cell cultures of 1 L, 3 L, 5 L, 10 L, 15 L, 40 L, 100 L, 500 L, 1000 L, 2000 L, 3000 L, 4000 L, 5000 L or larger. In some embodiments, the cell culture size can range from 0.01 L to 5000 L, from 0.1 L to 5000 L, from 1 L to 5000 L, from 5 L to 5000 L, from 40 L to 5000 L, from 100 L-5000 L, from 500 L to 5000 L, from 1000-5000 L, from 2000-5000 L, from 3000-5000 L, from 4000-5000 L, from 4500-5000 L, from 0.01 L to 1000 L, from 0.01-500 L, from 0.01-100 L, from 0.01-40 L, from 15-2000 L, from 40-1000 L, from 100-500 L, from 200-400 L, or any integer there between.

The RNA effector molecule(s) can be added during any phase of cell growth including, but not limited to, lag phase, stationary phase, early log phase, mid-log phase, late-log phase, exponential phase, or death phase. It is preferred that the cells are contacted with the RNA effector molecules prior to their entry into the death phase. In some embodiments, such as when targeting an apoptotic pathway, it may be desired to contact the cell in an earlier growth phase such as the lag phase, early log phase, mid-log phase or late-log phase (e.g., Bax/Bak inhibition). In other embodiments, it may be desired or acceptable to inhibit target gene expression at a later phase in the cell growth cycle (e.g., late-log phase or stationary phase), for example when growth-limiting products such as lactate are formed (e.g., LDH inhibition).

Compositions

Compositions for enhancing production of an immunogenic agent in cell culture by modulating the expression of a target gene in a host cell are also provided.

In one embodiment, the invention provides compositions containing a RNA effector molecule, as described herein, and an acceptable carrier. The composition containing the RNA effector molecule is useful for enhancing the production of an immunogenic agent by a cell by modulating the expression or activity of a target gene in the cell. Such compositions are formulated based on the mode of delivery. Provided herein are exemplary RNA effector molecules useful in improving the production of an immunogenic agent. In one embodiment, the RNA effector molecule in the composition is a siRNA. Alternatively, the RNA effector molecule in the composition is not a siRNA.

The RNA effector molecule compositions of the invention can be formulated as suspension in aqueous, non-aqueous, or mixed media and can be formulated in a lipid or non-lipid formulations, e.g., as described herein (see, e.g., the instant specification under section headings: ligand, lipid/oligonucleotide complexes, emulsions, surfactants, penetration enhancers, and additional carriers).

In one embodiment, the composition comprises at least one RNA effector molecule and a reagent that facilitates RNA effector molecule uptake, for example, an emulsion, a cationic lipid, a non-cationic lipid, a charged lipid, a liposome, an anionic lipid, a penetration enhancer, a transfection reagent or a modification to the RNA effector molecule for attachment, e.g., a ligand, a targeting moiety, a peptide, a lipophillic group, etc.

In some embodiments, the RNA effector molecule composition comprises a reagent that facilitates RNA effector molecule uptake which comprises “Lipid H” also known as lipid No. 200, “Lipid K” also known as lipid No. 201 or K8; “Lipid L” also known as lipid No. 202 or L8; “Lipid M” also known as lipid No. 203; “Lipid P” also known as lipid No. 204 or P8; or “Lipid R” also known as lipid No. 205, whose formulas are indicated as follows:

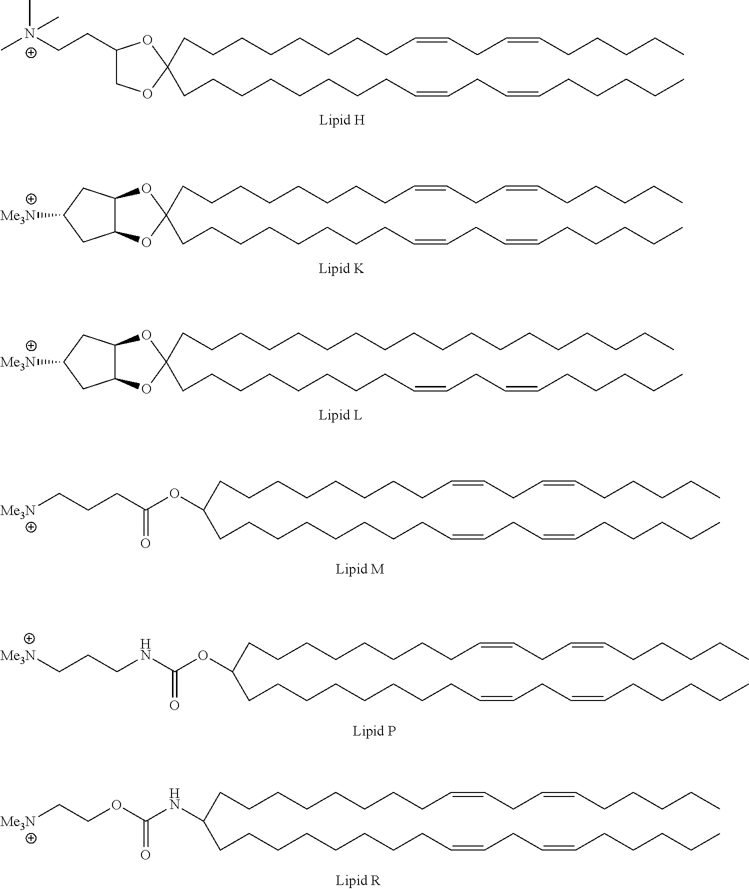

In another embodiment, the composition comprising a RNA effector molecule further comprises a growth medium, e.g. suitable for growth of the host cell. In one embodiment, the growth medium is a chemically defined media such as Biowhittaker® POWERCHO® (Lonza, Basel, Switzerland), HYCLONE PF CHO™ (Thermo Scientific, Fisher Scientific), GISCO®CD DG44 (Invitrogen, Carlsbad, Calif.), Medium M199 (Sigma-Aldrich), OPTIPRO™ SFM (Gibco), etc.). The RNA effector is ideally present in a concentration such that, when reconstituted, provides the optimal formulation.

In still another embodiment, the RNA effector molecule composition comprises a growth media supplement, e.g. an agent selected from the group consisting of essential amino acids (e.g., glutamine), 2-mercapto-ethanol, bovine serum albumin (BSA), lipid concentrate, cholesterol, catalase, insulin, human transferrin, superoxide dismutase, biotin, DL α-tocopherol acetate, DL α-tocopherol, vitamins (e.g., Vitamin A (acetate), choline chloride, D-calcium pantothenate, folic acid, Nicotinamide, pyridoxal hydrochloride, riboflavin, thiamine hydrochloride, i-Inositol), corticosterone, D-galactose, ethanolamine HCl, glutathione (reduced), L-carnitine HCl, linoleic acid, linolenic acid, progesterone, putrescine 2HCl, sodium selenite, T3 (triodo-1-thyronine), growth factors (e.g., EGF), iron, L-glutamine, L-alanyl-L-glutamine, sodium hypoxanthine, aminopterin and thymidine, arachidonic acid, ethyl Alcohol 100%, myristic acid, oleic acid, palmitic acid, almitoleic acid, pluronic F-68® (Invitrogen, Carlsbad, Calif.), stearic acid 10, TWEEN 80® nonionic surfactant (Invitrogen), sodium pyruvate, and glucose.

The RNA effector molecule composition can be provided in a sterile solution or lyophilized. In one embodiment the composition is packaged in discrete units by concentration and/or volume, e.g. to supply RNA effector molecule suitable for administration at various frequencies of administration and dosages, e.g. frequencies and dosages described herein.

In one embodiment, the composition is formulated for administration to cells according to a dosage regimen described herein, e.g., at a frequency of 6 hr, 12 hr, 24 hr, 36 hr, 48 hr, 72 hr, 84 hr, 96 hr, 108 hr, or more. Alternatively the composition is formulated at a dosage for continuous infusion.

Compositions containing two or more RNA effector molecules directed against separate target genes are also provided. The compositions can be used to enhance production of an immunogenic agent in cell culture by modulating expression of a first target gene and at least a second target gene in the cultured cells. In another embodiment, compositions containing two or more RNA effector molecules directed against the same target gene are provided.

Lipid/Oligonucleotide Complexes

In some embodiments, a reagent that facilitates RNA effector molecule uptake comprises a charged lipid, an emulsion, a liposome, a cationic or non-cationic lipid, an anionic lipid, a transfection reagent or a penetration enhancer as described herein. In one embodiment, the reagent that facilitates RNA effector molecule uptake used herein comprises a charged lipid as described in U.S. Application Ser. No. 61/267,419, filed 7 Dec. 2009.

The oligonucleotides of the present invention can be encapsulated within liposomes or can form complexes thereto, in particular to cationic liposomes. Alternatively, RNA effector molecules can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-20 alkyl ester (e.g., isopropylmyristate IPM), monoglyceride, diglyceride, or acceptable salts thereof.

In one embodiment, the RNA effector molecules are fully encapsulated in the lipid formulation (e.g., to form a SPLP, pSPLP, SNALP, or other nucleic acid-lipid particle). The term “SNALP” refers to a stable nucleic acid-lipid particle: a vesicle of lipids coating a reduced aqueous interior comprising a nucleic acid such as a RNA effector molecule or a plasmid from which a RNA effector molecule is transcribed. SNALPs are described, e.g., in U.S. Patent Pubs. No. 2006/0240093, No. 2007/0135372; No. 2009/0291131; U.S. patent application Ser. No. 12/343,342; No. 12/424,367. The term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SPLPs include “pSPLP,” which include an encapsulated condensing agent-nucleic acid complex as set forth in WO 00/03683. The particles in this embodiment typically have a mean diameter of about 50 nm to about 150 nm, or about 60 nm to about 130 nm, or about 70 nm to about 110 nm, or typically about 70 nm to about 90 nm, inclusive, and are substantially nontoxic. In addition, the nucleic acids when present in the nucleic acid-lipid particles of the present invention are resistant in aqueous solution to degradation with a nuclease. Nucleic acid-lipid particles and their method of preparation are reported in, e.g., U.S. Pat. No. 5,976,567; No. 5,981,501; No. 6,534,484; No. 6,586,410; No. 6,815,432; and WO 96/40964.

The lipid to drug ratio (mass/mass ratio) (e.g., lipid to dsRNA ratio) can be in ranges of from about 1:1 to about 50:1, from about 1:1 to about 25:1, from about 3:1 to about 15:1, from about 4:1 to about 10:1, from about 5:1 to about 9:1, or about 6:1 to about 9:1, inclusive.

A cationic lipid of the formulation can comprise at least one protonatable group having a pKa of from 4 to 15. The cationic lipid can be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA), 2,2-Dilinoleyl-4-dimethylaminoethyl-[1,3]-dioxolane, or a mixture thereof. The cationic lipid can comprise from about 20 mol % to about 70 mol %, inclusive, or about 40 mol % to about 60 mol %, inclusive, of the total lipid present in the particle. In one embodiment, cationic lipid can be further conjugated to a ligand.

A non-cationic lipid can be an anionic lipid or a neutral lipid, such as distearoyl-phosphatidylcholine (DSPC), dioleoylphosphatidylcholine (DOPC), dipalmitoyl-phosphatidylcholine (DPPC), dioleoylphosphatidylglycerol (DOPG), dipalmitoyl-phosphatidylglycerol (DPPG), dioleoyl-phosphatidylethanolamine (DOPE), palmitoyloleoyl-phosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE),16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be from about 5 mol % to about 90 mol %, inclusive, of about 10 mol %, to about 58 mol %, inclusive, if cholesterol is included, of the total lipid present in the particle.

The lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA can be, for example, a PEG-dilauryloxypropyl (C12), a PEG-dimyristyloxypropyl (C14), a PEG-dipalmityloxypropyl (C16), or a PEG-distearyloxypropyl (C18). The lipid that prevents aggregation of particles can be from 0 mol % to about 20 mol % or about 2 mol % of the total lipid present in the particle. In one embodiment, PEG lipid can be further conjugated to a ligand.

In some embodiments, the nucleic acid-lipid particle further includes a steroid such as, cholesterol at, e.g., about 10 mol % to about 60 mol %, inclusive, or about 48 mol % of the total lipid present in the particle.

In one embodiment, the lipid particle comprises a steroid, a PEG lipid and a cationic lipid of formula (I):

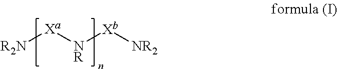

wherein each Xa and Xb, for each occurrence, is independently C₁₋₆ alkylene;

n is 0, 1, 2, 3, 4, or 5; each R is independently H,

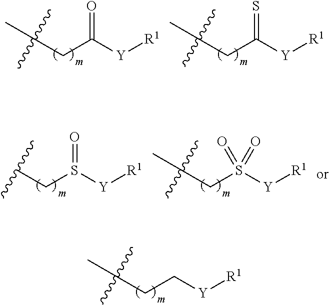

m is 0, 1, 2, 3 or 4; Y is absent, O, NR², or S; R¹ is alkyl alkenyl or alkynyl; each of which is optionally substituted with one or more substituents; and R² is H, alkyl alkenyl or alkynyl; each of which is optionally substituted each of which is optionally substituted with one or more substituents.

In one example, the lipidoid ND98.4HCl(MW 1487) (Formula 2), Cholesterol (Sigma-Aldrich), and PEG-Ceramide C16 (Avanti Polar Lipids) can be used to prepare lipid RNA effector molecule nanoparticles (e.g., LNP01 particles). Stock solutions of each in ethanol can be prepared as follows: ND98, 133 mg/mL; Cholesterol, 25 mg/mL; PEG-Ceramide C16, 100 mg/mL. The ND98, Cholesterol, and PEG-Ceramide C16 stock solutions can then be combined, for example, in a 42:48:10 molar ratio. The combined lipid solution can be mixed with aqueous RNA effector molecule (e.g., in sodium acetate pH 5) such that the final ethanol concentration is about 35% to 45% and the final sodium acetate concentration is about 100 mM to 300 mM, inclusive. Lipid RNA effector molecule nanoparticles typically form spontaneously upon mixing. Depending on the desired particle size distribution, the resultant nanoparticle mixture can be extruded through a polycarbonate membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. Buffer can be exchanged with, for example, phosphate buffered saline (PBS) at about pH 7, e.g., about pH 6.9, about pH 7.0, about pH 7.1, about pH 7.2, about pH 7.3, or about pH 7.4.

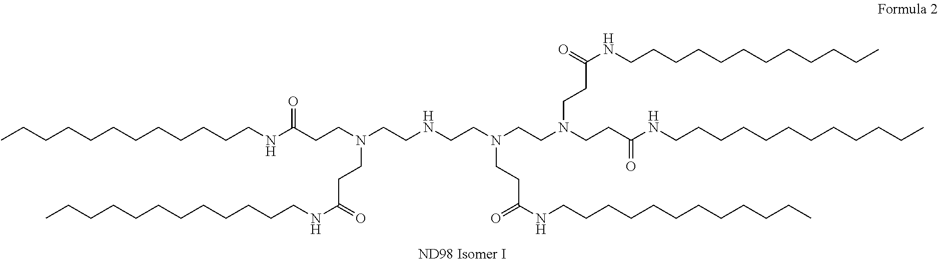 [SMILES: CCCCCCCCCCCCNC(=O)CCN(CCNCCN(CCC(=O)NCCCCCCCCCCCC)CCN(CCC(=O)NCCCCCCCCCCCC)CCC(=O)NCCCCCCCCCCCC)CCC(=O)NCCCCCCCCCCCC]

LNP01 formulations are described elsewhere, e.g., WO 2008/042973.

In one embodiment, the reagent that facilitates RNA effector molecule uptake used herein comprises a charged lipid as described in U.S. Application Ser. No. 61/267,419, filed 7 Dec. 2009, and U.S. Application Ser. No. 61/334,398, filed 13 May 2010. In various embodiments, the RNA effector molecule composition described herein comprises “Lipid H” also known as lipid No. 200, “Lipid K” also known as lipid No. 201 or K8; “Lipid L” also known as lipid No. 202 or L8; “Lipid M” also known as lipid No. 203; “Lipid P” also known as lipid No. 204 or P8; or “Lipid R” also known as lipid No. 205, whose formulas are indicated as follows:

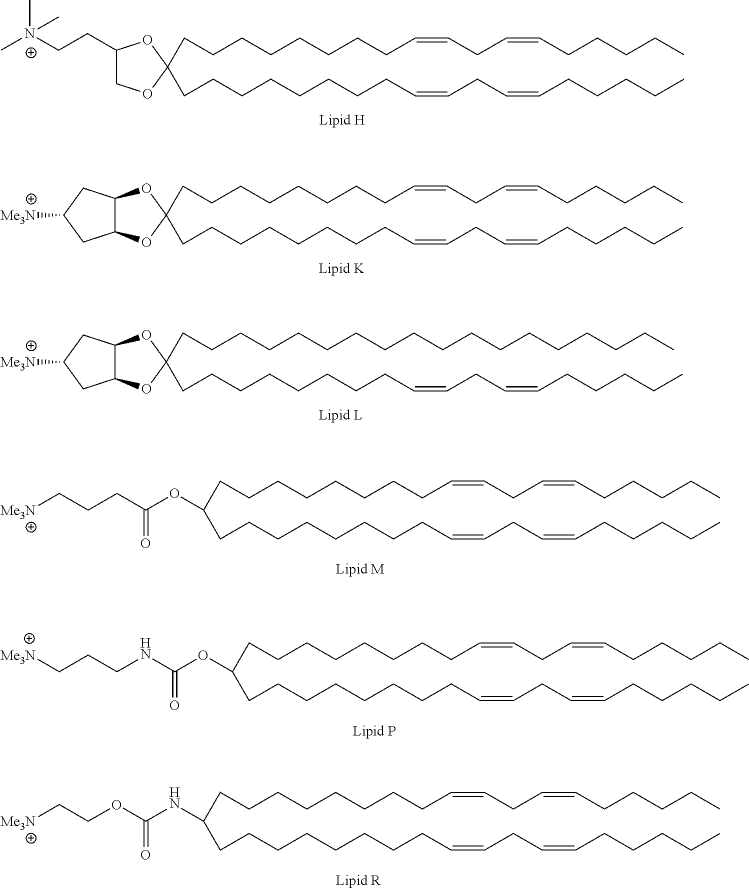

TABLE 19
Example lipid formulations

| Formulation | Cationic Lipid | Cationic Lipid | DOPE | Cholesterol |
|---|---|---|---|---|
| Number | Number | Mol % | % | % |
| 1 | 200 (Lipid H) | 48.08 | 51.92 | — |
| 2 | 200 (Lipid H) | 47.94 | 47.06 | 5 |
| 3 | 201 (Lipid K) | 45.56 | 54.44 | — |
| 4 (K8) | 201 (Lipid K) | 47.94 | 47.06 | 5 |
| 5 (L8) | 202 (Lipid L) | 47.94 | 47.06 | 5 |
| 6 | 203 (Lipid M) | 53.01 | 44.49 | 2.5 |
| 7 | 203 (Lipid M) | 47.94 | 47.06 | 5 |
| 8 (P8) | 204 (Lipid P) | 47.94 | 47.06 | 5 |
| 9 | 205 (Lipid R) | 47.94 | 47.06 | 5 |

In another embodiment, the RNA effector molecule composition described herein further comprises a lipid formulation comprising a lipid selected from the group consisting of Lipid H, Lipid K, Lipid L, Lipid M, Lipid P, and Lipid R, and further comprises a neutral lipid and a sterol. In particular embodiments, the lipid formulation comprises between approximately 25 mol %-100 mol % of the lipid. In another embodiment, the lipid formulation comprises between 0 mol %-50 mol % cholesterol. In still another embodiment, the lipid formulation comprises between 30 mol %-65 mol % of a neutral lipid. In particular embodiments, the lipid formulation comprises the relative mol % of the components as listed in Table 20 as follows:

TABLE 20
Example lipid formulae

| Series | Lipid (Mol %) | DOPE | Chol |
|---|---|---|---|
| 1 | 45.56 | 54.44 | 0 |
| 2 | 48.08 | 51.92 | 0 |
| 3 | 50.60 | 49.40 | 0 |
| 4 | 53.10 | 46.90 | 0 |
| 5 | 52.73 | 37.27 | 10 |
| 6 | 52.92 | 42.08 | 5 |
| 7 | 53.01 | 44.49 | 2.5 |
| 8 | 47.94 | 47.06 | 5 |

Additional exemplary lipid-siRNA formulations are as shown in Table 69, as follows:

TABLE 69
Lipid-siRNA formulations

| | | cationic lipid/non-cationic | |
|---|---|---|---|
| | | lipid/cholesterol/PEG-lipid | |
| | | conjugate | |
| | Cationic Lipid | Lipid:siRNA ratio | Process |
| SNALP | 1,2-Dilinolenyloxy-N,N- | DLinDMA/DPPC/Cholesterol/PEG-cDMA | |
| | dimethylaminopropane (DLinDMA) | (57.1/7.1/34.4/1.4) | |
| | | lipid:siRNA~7:1 | |
| SNALP-XTC | 2,2-Dilinoleyl-4-dimethylaminoethyl-[1,3]- | XTC/DPPC/Cholesterol/PEG-cDMA | |
| | dioxolane (XTC) | 57.1/7.1/34.4/1.4 | |
| | | lipid:siRNA~7:1 | |
| LNP05 | 2,2-Dilinoleyl-4-dimethylaminoethyl-[1,3]- | XTC/DSPC/Cholesterol/PEG-DMG | Extrusion |
| | dioxolane (XTC) | 57.5/7.5/31.5/3.5 | |
| | | lipid:siRNA~6:1 | |
| LNP06 | 2,2-Dilinoleyl-4-dimethylaminoethyl-[1,3]- | XTC/DSPC/Cholesterol/PEG-DMG | Extrusion |
| | dioxolane (XTC) | 57.5/7.5/31.5/3.5 | |
| | | lipid:siRNA~11:1 | |
| LNP07 | 2,2-Dilinoleyl-4-dimethylaminoethyl-[1,3]- | XTC/DSPC/Cholesterol/PEG-DMG | In-line |
| | dioxolane (XTC) | 60/7.5/31/1.5, | mixing |
| | | lipid:siRNA~6:1 | |
| LNP08 | 2,2-Dilinoleyl-4-dimethylaminoethyl-[1,3]- | XTC/DSPC/Cholesterol/PEG-DMG | In-line |
| | dioxolane (XTC) | 60/7.5/31/1.5, | mixing |
| | | lipid:siRNA~11:1 | |
| LNP09 | 2,2-Dilinoleyl-4-dimethylaminoethyl-[1,3]- | XTC/DSPC/Cholesterol/PEG-DMG | In-line |
| | dioxolane (XTC) | 50/10/38.5/1.5 | mixing |
| | | Lipid:siRNA 10:1 | |
| LNP10 | (3aR,5s,6aS)-N,N-dimethyl-2,2-di((9Z,12Z)- | ALN100/DSPC/Cholesterol/PEG-DMG | In-line |
| | octadeca-9,12-dienyl)tetrahydro-3aH- | 50/10/38.5/1.5 | mixing |
| | cyclopenta[d][1,3]dioxol-5-amine (ALN100) | Lipid:siRNA 10:1 | |
| LNP11 | (6Z,9Z,28Z,31Z)-heptatriaconta-6,9,28,31- | MC-3/DSPC/Cholesterol/PEG-DMG | In-line |
| | tetraen-19-yl 4-(dimethylamino)butanoate | 50/10/38.5/1.5 | mixing |
| | (MC3) | Lipid:siRNA 10:1 | |
| LNP12 | 1,1′-(2-(4-(2-((2-(bis(2- | Tech G1/DSPC/Cholesterol/PEG-DMG | In-line |
| | hydroxydodecyl)amino)ethyl)(2- | 50/10/38.5/1.5 | mixing |
| | hydroxydodecyl)amino)ethyl)piperazin-1- | Lipid:siRNA 10:1 | |
| | yl)ethylazanediyl)didodecan-2-ol (Tech G1) | | |

LNP09 formulations and XTC comprising formulations are described, e.g., in U.S. Provisional Ser. No. 61/239,686, filed Sep. 3, 2009. LNP11 formulations and MC3 comprising formulations are described, e.g., in U.S. Provisional Ser. No. 61/244,834, filed Sep. 22, 2009. LNP12 formulations and TechG1 comprising formulations are described, e.g., in U.S. Provisional Ser. No. 61/175,770, filed May 5, 2009.

Formulations prepared by either the standard or extrusion-free method can be characterized in similar manners. For example, formulations are typically characterized by visual inspection. They should be whitish translucent solutions free from aggregates or sediment. Particle size and particle size distribution of lipid-nanoparticles can be measured by light scattering using, for example, a Malvern Zetasizer Nano ZS (Malvern, Pa.). Particles should be about 20 nm to 300 nm, such as 40 nm to 100 nm in size. The particle size distribution should be unimodal. The total RNA effector molecule concentration in the formulation, as well as the entrapped fraction, is estimated using a dye exclusion assay. A sample of the formulated RNA effector molecule can be incubated with a RNA-binding dye, such as Ribogreen (Molecular Probes) in the presence or absence of a formulation disrupting surfactant, e.g., 0.5% Triton-X100. The total RNA effector molecule in the formulation can be determined by the signal from the sample containing the surfactant, relative to a standard curve. The entrapped fraction is determined by subtracting the “free” RNA effector molecule content (as measured by the signal in the absence of surfactant) from the total RNA effector molecule content. Percent entrapped RNA effector molecule is typically >85%. For lipid nanoparticle formulation, the particle size is at least 30 nm, at least 40 nm, at least 50 nm, at least 60 nm, at least 70 nm, at least 80 nm, at least 90 nm, at least 100 nm, at least 110 nm, or at least 120 nm. The suitable range is typically about at least 50 nm to about at least 110 nm, about at least 60 nm to about at least 100 nm, or about at least 80 nm to about at least 90 nm, inclusive.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo. In order to cross intact cell membranes, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include: liposomes obtained from natural phospholipids are biocompatible and biodegradable; liposomes can incorporate a wide range of water and lipid soluble drugs; and liposomes can protect encapsulated drugs in their internal compartments from metabolism and degradation. See, e.g., Wang et al., DRUG DELIV. PRINCIPLES & APPL. (John Wiley & Sons, Hoboken, N.J., 2005); Rosoff, 1988. Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent can act. Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Liposomes fall into two broad classes. Cationic liposomes are positively charged liposomes which interact with the negatively charged polynucleotide molecules to form a stable complex. The positively charged polynucleotide/liposome complex binds to the negatively charged cell surface and is internalized in an endosome. Due to the acidic pH within the endosome, the liposomes are ruptured, releasing their contents into the cell cytoplasm. Wang et al., 147 Biochem. Biophys. Res. Commun, 980-85 (1987).

Liposomes which are pH-sensitive or negatively-charged, entrap polynucleotide rather than complex with it. Because both the polynucleotide and the lipid are similarly charged, repulsion rather than complex formation occurs. Nevertheless, some polynucleotide is entrapped within the aqueous interior of these liposomes. pH-sensitive liposomes have been used to deliver DNA encoding the thymidine kinase gene to cell monolayers in culture. Expression of the exogenous gene was detected in the target cells. Zhou et al., 19 J. Controlled Rel. 269-74 (1992).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside GM1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES). Allen et al., 223 FEBS Lett. 42 (1987); Wu et al., 53 Cancer Res. 3765 (1993).

Various liposomes comprising one or more glycolipids are known in the art. Papahadjopoulos et al. (507 Ann. N.Y. Acad. Sci. 64 (1987)), reported the ability of monosialoganglioside GM1, galactocerebroside sulfate and phosphatidylinositol to improve blood half-lives of liposomes. These findings were expounded upon by Gabizon et al. (85 PNAS 6949 (1988)). U.S. Pat. No. 4,837,028 and WO 88/04924, both to Allen et al., disclose liposomes comprising (1) sphingomyelin and (2) the ganglioside GM1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (53 Bull. Chem. Soc. Jpn. 2778 (1980)) described liposomes comprising a nonionic detergent, 2C1215G, that contains a PEG moiety. Illum et al. (167 FEBS Lett. 79 (1984)), noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. No. 4,426,330 and No. 4,534,899). In addition, antibodies can be conjugated to a polyakylene derivatized liposome (see e.g., U.S. Application Pub. No. 2008/0014255). Klibanov et al. (268 FEBS Lett. 235 (1990)), described experiments demonstrating that liposomes comprising phosphatidylethanolamine (PE) derivatized with PEG or PEG stearate have significant increases in blood circulation half-lives. Blume et al. (1029 Biochim. Biophys. Acta 1029, (1990)), extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. 0 445 131 B1 and WO 90/04384 to Fisher.

Liposome compositions containing 1 mol % to 20 mol % of PE derivatized with PEG, and methods of use thereof, are described by Woodle et al. (U.S. Pat. No. 5,013,556; No. 5,356,633) and Martin et al. (U.S. Pat. No. 5,213,804; European Patent No. 0 496813 B1). Liposomes comprising a number of other lipid-polymer conjugates are disclosed in WO 91/05545 and U.S. Pat. No. 5,225,212 and in WO 94/20073. Liposomes comprising PEG-modified ceramide lipids are described in WO 96/10391. U.S. Pat. No. 5,540,935 and No. 5,556,948 describe PEG-containing liposomes that can be further derivatized with functional moieties on their surfaces. Methods and compositions relating to liposomes comprising PEG can be found in, e.g., U.S. Pat. No. 6,049,094; No. 6,224,903; No. 6,270,806; No. 6,471,326; No. 6,958,241.

As noted above, liposomes can, optionally, be prepared to contain surface groups, such as antibodies or antibody fragments, small effector molecules for interacting with cell-surface receptors, antigens, and other like compounds, and these groups can facilitate delivery of liposomes and their contents to specific cell populations. Such ligands can be included in the liposomes by including in the liposomal lipids a lipid derivatized with the targeting molecule, or a lipid having a polar-head chemical group that can be derivatized with the targeting molecule in preformed liposomes. Alternatively, a targeting moiety can be inserted into preformed liposomes by incubating the preformed liposomes with a ligand-polymer-lipid conjugate.

Lipids can be derivatized using a variety of targeting moieties, such as ligands, cell surface receptors, glycoproteins, vitamins (e.g., riboflavin) and monoclonal antibodies by covalently attaching the ligand to the free distal end of a hydrophilic polymer chain, which is attached at its proximal end to a vesicle-forming lipid. There are a wide variety of techniques for attaching a selected hydrophilic polymer to a selected lipid and activating the free, unattached end of the polymer for reaction with a selected ligand, and as noted above, the hydrophilic polymer polyethyleneglycol (PEG) has been studied widely. Allen et al., 1237 Biochem. Biophys. Acta 99-108 (1995); Zalipsky, 4 Bioconj. Chem. 296-99 (1993); Zalipsky et al., 353 FEBS Lett. 1-74 (1994); Zalipsky et al., Bioconj. Chem. 705-08 (1995); Zalipsky, in STEALTH LIPOSOMES (Lasic & Martin, eds. CRC Press, Boca Raton, Fla., 1995).

A number of liposomes comprising nucleic acids are known in the art, such as methods for encapsulating high molecular weight nucleic acids in liposomes. WO 96/40062. U.S. Pat. No. 5,264,221 to Tagawa et al. discloses protein-bonded liposomes and asserts that the contents of such liposomes can include a dsRNA. U.S. Pat. No. 5,665,710 describes certain methods of encapsulating oligodeoxynucleotides in liposomes. WO 97/04787 refers to liposomes comprising dsRNAs targeted to the raf gene. In addition, methods for preparing a liposome composition comprising a nucleic acid can be found in, e.g., U.S. Pat. No. 6,011,020; No. 6,074,667; No. 6,110,490; No. 6,147,204; No. 6,271,206; No. 6,312,956; No. 6,465,188; No. 6,506,564; No. 6,750,016; No. 7,112,337.

Transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes are adaptable to the environment in which they are used, e.g., they are self-optimizing, self-repairing, frequently reach their targets without fragmenting, and often self-loading. To make transfersomes it is possible to add surface edge-activators, usually surfactants, to a standard liposomal composition.

Encapsulated nanoparticles can also be used for delivery of RNA effector molecules. Examples of such encapsulated nanoparticles include those created using yeast cell wall particles (YCWP). For example, glucan-encapsulated siRNA particles (GeRPs) are payload delivery systems made up of a yeast cell wall particle (YCWP) exterior and a multilayered nanoparticle interior, wherein the multilayered nanoparticle interior has a core comprising a payload complexed with a trapping agent. Glucan-encapsulated delivery systems, such as those described in U.S. patent application Ser. No. 12/260,998, filed Oct. 29, 2008, can be used to deliver siRNA duplexes to achieve silencing in vitro and in vivo.

Emulsions

The compositions of the present invention can be prepared and formulated as emulsions. Emulsions are typically heterogenous systems of one liquid dispersed in another in the form of droplets usually exceeding 0.1 μm in diameter. See, e.g., Ansel's PHARM. DOSAGE FORMS & DRUG DELIV. SYS. (8th ed. Allen et al., eds., Lippincott Williams & Wilkins, NY, 2004); Idson, in 1 PHARM. DOSAGE FORMS 199 (Lieberman et al., eds., Marcel Dekker, Inc., NY, 1988); Rosoff, in 1 PHARM. DOSAGE FORMS 245 (Lieberman et al., eds., Marcel Dekker, Inc., NY, 1988); Block in 2 PHARM. DOSAGE FORMS 335 (Lieberman et al., eds., Marcel Dekker, Inc., NY, 1988); Higuchi et al., in REMINGTON'S PHARM. SCI. 301 (Mack Publishing Co., Easton, Pa., 1985). Emulsions are often biphasic systems comprising two immiscible liquid phases intimately mixed and dispersed with each other.

In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution in either the aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids. See, e.g., ANSEL'S PHARM. DOSAGE FORMS & DRUG DELIV. SYS., 2004; Idson, in PHARM. DOSAGE FORMS, 1988.

Synthetic surfactants, also known as surface active agents, have found wide applicability in the formulation of emulsions and have been reviewed in the literature. See, e.g., ANSEL'S PHARM. DOSAGE FORMS & DRUG DELIV. SYS., 2004; Idson, in PHARM. DOSAGE FORMS, 1988; Rieger, in PHARM. DOSAGE FORMS, 1988. Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric. See, e.g., ANSEL'S PHARM. DOSAGE FORMS & DRUG DELIV. SYS., 2004; Idson, in PHARM. DOSAGE FORMS, 1988; Rieger, in PHARM. DOSAGE FORMS, 1988.

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants. Block, in 1 PHARM. DOSAGE FORMS 335 (Lieberman et al., eds., Marcel Dekker, Inc., NY, 1988); Idson, in PHARM. DOSAGE FORMS (1988).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Because emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

In one embodiment, the compositions of RNA effector molecules and nucleic acids are formulated as microemulsions. A microemulsion can be defined as a system of water, oil and amphiphile which is a single optically isotropic and thermodynamically stable liquid solution. See, e.g., ANSEL'S PHARM. DOSAGE FORMS & DRUG DELIV. SYS. (8th ed., Allen et al, eds., Lippincott Williams & Wilkins, NY, 2004); Rosoff, in PHARM. DOSAGE FORMS, 1988. Typically, microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules. Leung & Shah, in CONTROLLED RELEASE DRUGS: POLYMERS & AGGREGATE SYS. 185-215 (Rosoff, ed., VCH Publishers, NY, 1989). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules. Schott, in REMINGTON'S PHARM. SCI. 271 (1985).

The phenomenological approach utilizing phase diagrams has been extensively studied and has yielded a comprehensive knowledge, to one skilled in the art, of how to formulate microemulsions. See, e.g., ANSEL'S PHARM. DOSAGE FORMS & DRUG DELIV. SYS. (8th ed., Allen et al, eds., Lippincott Williams & Wilkins, NY, 2004); Rosoff, 1988; Block, 1988. Compared to conventional emulsions, microemulsions offer the advantage of solubilizing water-insoluble drugs in a formulation of thermodynamically stable droplets that are formed spontaneously.

Microemulsions can include surfactants, discussed further herein, not limited to ionic surfactants, non-ionic surfactants, Brij 96, polyoxyethylene oleyl ethers, polyglycerol fatty acid esters, tetraglycerol monolaurate (ML310), tetraglycerol monooleate (M0310), hexaglycerol monooleate (PO310), hexaglycerol pentaoleate (PO500), decaglycerol monocaprate (MCA750), decaglycerol monooleate (M0750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions afford advantages of better drug solubilization, protection of drug from enzymatic hydrolysis, possible enhancement of drug absorption due to surfactant-induced alterations in membrane fluidity and permeability, ease of preparation, and decreased toxicity. See, e.g., U.S. Pat. No. 6,191,105; No. 7,063,860; No. 7,070,802; No. 7,157,099; Constantinides et al., 11 Pharm. Res. 1385 (1994); Ho et al., 85 J. Pharm. Sci. 138-43 (1996). Often, microemulsions can form spontaneously when their components are brought together at ambient temperature. This can be particularly advantageous when formulating thermolabile drugs, peptides or RNA effector molecules.

Microemulsions of the present invention can also contain additional components and additives such as sorbitan monostearate (Grill 3), Labrasol, and penetration enhancers to improve the properties of the formulation and to enhance the absorption of the RNA effector molecules and nucleic acids of the present invention. Penetration enhancers used in the microemulsions of the present invention can be classified as belonging to one of five broad categories—surfactants, fatty acids, bile salts, chelating agents, and non-chelating non-surfactants. Lee et al., Crit. Rev. Therapeutic Drug Carrier Sys. 92 (1991).

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Surfactants

In some embodiments, RNA effector molecules featured in the invention are formulated in conjunction with one or more penetration enhancers, surfactants and/or chelators. Suitable surfactants include fatty acids and/or esters or salts thereof, bile acids and/or salts thereof. Suitable bile acids/salts include chenodeoxycholic acid (CDCA) and ursodeoxychenodeoxy-cholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations. See e.g., Malmsten, SURFACTANTS & POLYMERS IN DRUG DELIV. (Informa Health Care, NY, 2002); Rieger, in PHARM. DOSAGE FORMS 285 (Marcel Dekker, Inc., NY, 1988).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class. If the surfactant molecule has the ability to carry either a positive or negative charge, the surfactant is classified as amphoteric. Amphoteric surfactants include acrylic acid derivatives, substituted alkylamides, N-alkylbetaines and phosphatides.

Penetration Enhancers

In one embodiment, the present invention employs various penetration enhancers to effect the efficient delivery of nucleic acids, particularly RNA effector molecules, to the cell. Most drugs are present in solution in both ionized and nonionized forms. Usually, only lipid soluble or lipophilic drugs readily cross cell membranes. It has been discovered that even non-lipophilic drugs can cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers can be classified as belonging to one of five broad categories: surfactants, fatty acids, bile salts, chelating agents, and non-chelating non-surfactants. See, e.g., Malmsten, 2002; Lee et al., Crit. Rev. Therapeutic Drug Carrier Sys. 92 (1991).

In connection with the present invention, penetration enhancers include surfactants (or “surface-active agents”), which are chemical entities that, when dissolved in an aqueous solution, reduce the surface tension of the solution or the interfacial tension between the aqueous solution and another liquid, with the result that absorption of RNA effector molecules through cellular membranes and other biological barriers is enhanced. In addition to bile salts and fatty acids, these penetration enhancers include, for example, sodium lauryl sulfate, polyoxyethylene-9-lauryl ether and polyoxyethylene-20-cetyl ether) (see, e.g., Malmsten, 2002; Lee et al., 1991); and perfluorochemical emulsions, such as FC-43 (Takahashi et al., 40 J. Pharm. Pharmacol. 252 (1988)).

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacyclo-heptan-2-one, acylcarnitines, acylcholines, C1-20 alkyl esters thereof (e.g., methyl, isopropyl and t-butyl), and mono- and di-glycerides thereof (i.e., oleate, laurate, caprate, myristate, palmitate, stearate, linoleate, etc.). See, e.g., Touitou et al., ENHANCEMENT IN DRUG DELIV. (CRC Press, Danvers, Mass., 2006); Lee et al., 1991; Muranishi, 7 Crit. Rev. Therapeutic Drug Carrier Sys. 1-33 (1990); E1 Hariri et al., 44 J. Pharm. Pharmacol. 651-54 (1992).

The physiological role of bile includes the facilitation of dispersion and absorption of lipids and fat-soluble vitamins. See, e.g., Malmsten, 2002; Brunton, Chapt. 38 in GOODMAN & GILMAN'S PHARMACOLOGICAL BASIS THERAPEUTICS, 9TH ED. 934-35 (Hardman et al., eds., McGraw-Hill, NY, 1996). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (see e.g., Malmsten, 2002; Lee et al., 1991; Swinyard, Chapt. 39 in REMINGTON'S PHARM. SCI., 18th Ed. 782-83 (Gennaro, ed., Mack Publishing Co., Easton, Pa., 1990); Muranishi, 1990; Yamamoto et al., 263 J. Pharm. Exp. Ther. 25 (1992); Yamashita et al., 79 J. Pharm. Sci. 579-83 (1990).

Chelating agents, as used in connection with the present invention, can be defined as compounds that remove metallic ions from solution by forming complexes therewith, with the result that absorption of RNA effector molecules through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents. Jarrett, 618 J. Chromatogr. 315-39 (1993). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines). See, e.g., Katdare et al., EXCIPIENT DEVEL. PHARM. BIOTECH. & DRUG DELIV. (CRC Press, Danvers, Mass., 2006); Lee et al., 1991; Muranishi, 1990; Buur et al., 14 J. Control Rel. 43-51 (1990).

As used herein, non-chelating non-surfactant penetration enhancing compounds can be defined as compounds that demonstrate insignificant activity as chelating agents or as surfactants but that nonetheless enhance absorption of RNA effector molecules through the alimentary mucosa. See e.g., Muranishi, 1990. This class of penetration enhancers include, for example, unsaturated cyclic ureas, 1-alkyl- and 1-alkenylazacyclo-alkanone derivatives (Lee et al., 1991); and non-steroidal anti-inflammatory agents such as diclofenac sodium, indomethacin and phenylbutazone (Yamashita et al., 1987).

Agents that enhance uptake of RNA effector molecules at the cellular level can also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), are also known to enhance the cellular uptake of dsRNAs. Examples of commercially available transfection reagents include, for example LIPOFECTAMINE™, LIPOFECTAMINE 2000™, 293FECTIN™, CELLFECTIN™, DMRIE-C™, FREESTYLE™ MAX, LIPOFECTAMINE™2000 CD, LIPOFECTAMINE™, RNAiMAX, OLIGOFECTAMINE™, and OPTIFECT™ transfection reagents (each from Invitrogen); and X-tremeGENE Q2 Transfection Reagent (Roche Applied Science; Grenzacherstrasse, Switzerland), DOTAP Liposomal Transfection Reagent (Avante Polar Lipids, Inc., Alabaster, Ala.), DOSPER Liposomal Transfection Reagent (Roche), or FuGENE® (Promega; Madison, Wis.) or TRANSFECTAM® Reagent (Promega), TRANSFAST™ Transfection Reagent (Promega), TFX™-20 Reagent (Promega), TFX™-50 Reagent (Promega); DREAMFECT™ (OZ Biosciences; Marseille, France), EcoTransfect (OZ Biosciences); TRANSPASS® D1 Transfection Reagent (New England Biolabs; Ipswich, Mass.); LYOVEC™/LIPOGENT™ (InvivoGen; San Diego, Calif.); PerFectin Transfection Reagent (Genlantis; San Diego, Calif.), NEUROPORTER Transfection Reagent (Genlantis), GENEPORTER Transfection reagent (Genlanti), GENEPORTER 2 Transfection reagent (Genlantis), CYTOFECTIN Transfection Reagent (Genlantis), BACULOPORTER Transfection Reagent (Genlantis), TROGANPORTER™ transfection reagent (Genlantis); RIBOFECT (Bioline; Taunton, Mass., U.S.), PLASFECT (Bioline), UNIFECTOR (B-Bridge International; Mountain View, Calif.), SUREFECTOR (B-Bridge International), or HIFECT™ (B-Bridge Int'l), among others.

Additional Carriers

Other agents can be utilized to enhance the penetration of the administered nucleic acids, including glycols such as ethylene glycol and propylene glycol, pyrrols such as 2-pyrrol, azones, and terpenes such as limonene and menthone.

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal.

The compositions of the present invention can additionally contain other adjunct components so long as such materials, when added, do not unduly interfere with the biological activities of the components of the compositions of the present invention. The formulations can be sterilized and, if desired, mixed with auxiliary agents that do not deleteriously interact with the RNA effector molecules of the formulation.

Aqueous suspensions can contain substances which increase the viscosity of the suspension including, for example, sodium carboxymethylcellulose, sorbitol and/or dextran. The suspension can also contain stabilizers.

Toxicity and therapeutic efficacy of such compounds can be determined by standard pharmaceutical procedures in cell cultures or in cells, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are particularly useful. The data obtained from cell culture assays and animal studies can be used in formulating a range of dosages for use in the instant methods. The dosage of compositions featured in the invention lies generally within a range of concentrations that includes the ED₅₀ with little or no toxicity. The dosage can vary within this range depending upon the dosage form employed and the route of administration utilized.

In yet another aspect, the invention provides a method for inhibiting the expression of a target gene in a host cell by administering a composition featured in the invention to the host cell such that expression of the target gene is decreased for an extended duration, e.g., at least two, three, four days or more, e.g., one week, two weeks, three weeks, or four weeks or longer. The effect of the decreased expression of the target gene preferably results in a decrease in levels of the protein encoded by the target gene by at least 10%, at least 15%, at least 20%, at least 25%, at least 30%, at least 40%, at least 50%, or at least 60%, or more, as compared to pretreatment levels.

### VII. KITS AND ASSAYS

In some embodiments, kits are provided for testing the effect of a RNA effector molecule or a series of RNA effector molecules on the production of an immunogenic agent by the cell, where the kits comprise a substrate having one or more assay surfaces suitable for culturing cells under conditions that allow production of an immunogenic agent. In some embodiments, the exterior of the substrate comprises wells, indentations, demarcations, or the like at positions corresponding to the assay surfaces. In some embodiments, the wells, indentations, demarcations, or the like retain fluid, such as cell culture media, over the assay surfaces.

In some embodiments, the assay surfaces on the substrate are sterile and are suitable for culturing host cells under conditions representative of the culture conditions during large-scale (e.g., industrial scale) production of the immunogenic agent. Advantageously, kits provided herein offer a rapid, cost-effective means for testing a wide-range of agents and/or conditions on the production of an immunogenic agent, allowing the cell culture conditions to be established prior to full-scale production of the immunogenic agent.

In some embodiments, one or more assay surfaces of the substrate comprise a concentrated test agent, such as a RNA effector molecule, such that the addition of suitable media to the assay surfaces results in a desired concentration of the RNA effector molecule surrounding the assay surface. In some embodiments, the RNA effector molecules can be printed or ingrained onto the assay surface, or provided in a lyophilized form, e.g., within wells, such that the effector molecules can be reconstituted upon addition of an appropriate amount of media. In some embodiments, the RNA effector molecules are reconstituted by plating cells onto assay surfaces of the substrate.

In some embodiments, kits provided herein further comprise cell culture media suitable for culturing a cell under conditions allowing for the production of an immunogenic agent of interest. The media can be in a ready to use form or can be concentrated (e.g., as a stock solution), lyophilized, or provided in another reconstitutable form.

In further embodiments, kits provided herein further comprise one or more reagents suitable for detecting production of the immunogenic agent by the cell, cell culture, or tissue culture. In further embodiments, the reagent(s) are suitable for detecting a property of the cell, such as maximum cell density, cell viability, or the like, which is indicative of production of the desired immunogenic agent. In some embodiments, the reagent(s) are suitable for detecting the immunogenic agent or a property thereof, such as the in vitro or in vivo biological activity, homogeneity, or structure of the immunogenic agent.

In some embodiments, one or more assay surfaces of the substrate further comprise a carrier for which facilitates uptake of RNA effector molecules by cells. Carriers for RNA effector molecules are known in the art and are described herein. For example, in some embodiments, the carrier is a lipid formulation such as LIPOFECTAMINE™ transfection reagent (Invitrogen; Carlsbad, Calif.) or a related formulation. Examples of such carrier formulations are described herein. In some embodiments, the reagent that facilitates RNA effector molecule uptake comprises a charged lipid, an emulsion, a liposome, a cationic or non-cationic lipid, an anionic lipid, a transfection reagent or a penetration enhancer as described throughout the application herein. In particular embodiments, the reagent that facilitates RNA effector molecule uptake comprises a charged lipid as described in U.S. Application Ser. No. 61/267,419, filed on Dec. 7, 2009.

In some embodiments, one or more assay surfaces of the substrate comprise a RNA effector molecule or series of RNA effector molecules and a carrier, each in concentrated form, such that plating test cells onto the assay surface(s) results in a concentration the RNA effector molecule(s) and the carrier effective for facilitating uptake of the RNA effector molecule(s) by the cells and modulation of the expression of one or more genes targeted by the RNA effector molecules.

In some embodiments, the substrate further comprises a matrix which facilitates 3-dimensional (3-D) cell growth and/or production of the immunogenic agent by the cells. In further embodiments, the matrix facilitates anchorage-dependent growth of cells. Non-limiting examples of matrix materials suitable for use with various kits described herein include agar, agarose, methylcellulose, alginate hydrogel (e.g., 5% alginate+5% collagen type I), chitosan, hydroactive hydrocolloid polymer gels, polyvinyl alcohol-hydrogel (PVA-H), polylactide-co-glycolide (PLGA), collagen vitrigel, PHEMA (poly(2-hydroxylmethacrylate)) hydrogels, PVP/PEO hydrogels, BD PURAMATRIX™ hydrogels, and copolymers of 2-methacryloyloxyethyl phosphorylcholine (MPC).

In some embodiments, the substrate comprises a microarray plate, a biochip, or the like which allows for the high-throughput, automated testing of a range of test agents, conditions, and/or combinations thereof on the production of an immunogenic agent by cultured cells. For example, the substrate can comprise a 2-dimensional microarray plate or biochip having m columns and n rows of assay surfaces (e.g., residing within wells) which allow for the testing of m×n combinations of test agents and/or conditions (e.g., on a 24, 96 or 384-well microarray plate). The microarray substrates are preferably designed such that all necessary positive and negative controls can be carried out in parallel with testing of the agents and/or conditions.

In further embodiments, kits are provided comprising one or more microarray substrates seeded with a set of RNA effector molecules designed to modulate a particular pathway, function, or property of a cell which affects the production of the immunogenic agent. For example, in some embodiments, the RNA effector molecules are directed against target genes comprising a pathway involved in the expression, folding, secretion, or post-translational modification of a recombinant immunogenic agent by the cell.

In further embodiments, kits are provided herein comprising one or more microarray substrates seeded with a set of RNA effector molecules designed to address a particular problem or class of problems associated with the production of an immunogenic agent in cell-based systems. For example, in some embodiments, the RNA effector molecules are directed against target genes expressed by latent or endogenous viruses; or involved in cell processes, such as cell cycle progression, cell metabolism or apoptosis which inhibit or interfere production or purification of the immunogenic agent. In further embodiments, the RNA effector molecules are directed against target genes that mediate enzymatic degradation, aggregation, misfolding, or other processes that reduce the activity, homogeneity, stability, and/or other qualities of the immunogenic agent. In yet further embodiments, the effector molecules are directed against target genes that affect the infectivity of exogenous or adventitious contaminating microbes. In one embodiment, the immunogenic agent includes a glycoprotein, and the RNA effector molecules are directed against target genes involved in glycosylation (e.g., fucosylation) and/or proteolytic processing of glycoproteins by the host cell. In another embodiment, the immunogenic agent is a multi-subunit recombinant protein and the RNA effector molecules are directed against target genes involved in the folding and/or secretion of the protein by the host cell. In another embodiment, the RNA effector molecules are directed against target genes involved in post-translation modification of the immunogenic agent in the cells, such as methionine oxidation, glycosylation, disulfide bond formation, pyroglutamation and/or protein deamidation.

In some embodiments, kits provided herein allow for the selection or optimization of a combination o two or more factors in production of the immunogenic agent. For example, the kits can allow for the selection of a suitable RNA effector molecule from among a series of candidate RNA effector molecules as well as a concentration of the RNA effector molecule. In further embodiments, kits provided herein allow for the selection of a first RNA effector molecule from a first series of candidate RNA effector molecules and a second RNA effector molecule from a second series of candidate RNA effector molecules. In some embodiments, the first and/or second series of candidate RNA effector molecules are directed against a common target gene. In further embodiments, the first and/or second series of RNA effector molecules are directed against two or more functionally related target genes or two or more target genes of a common host cell pathway.

In another embodiment, a kit for enhancing production of an immunogenic agent in a cell, comprising at least a first RNA effector molecule, a portion of which is complementary to at least a first target gene of a latent or endogenous virus; a second RNA effector molecule, a portion of which is complementary to at least a secon target gene of the cellular immune response; and, optionally, a third RNA effector molecule, a portion of which is complementary to at least a third target gene of a cellular process. For example, the first target gene is an ERV env gene, the second target gene is a IFNAR1 or IFNB gene, and the third target gene is a PTEN, BAK1, FN1, or LDHA gene. The kit can further comprise at least additional RNA effector molecule that targets a cellular process including, but not limited to, carbon metabolism and transport, apoptosis, RNAi uptake and/or efficiency, reactive oxygen species production, cell cycle control, protein folding, pyroglutamation protein modification, deamidase, glycosylation, disulfide bond formation, protein secretion, gene amplification, viral replication, viral infection, viral particle release, control of cellular pH, and protein production.

In yet another aspect, the invention provides a method for inhibiting the expression of a target gene in a cell. The method includes administering a composition featured in the invention to the cell such that expression of the target gene is decreased, such as for an extended duration, e.g., at least two, three, four days or more. The RNA effector molecules useful for the methods and compositions featured in the invention specifically target RNAs (primary or processed) of the target gene. Compositions and methods for inhibiting the expression of these target genes using RNA effector molecules can be prepared and performed as described herein.

The present invention may be as defined in any one of the following numbered paragraphs:

1. A method for producing an immunogenic agent in a large scale host cell culture, comprising: (a) contacting a host cell in a large scale host cell culture with at least a first RNA effector molecule, a portion of which is complementary to at least one target gene of a host cell, (b) maintaining the host cell culture for a time sufficient to modulate expression of the at least one first target gene, wherein the modulation of expression improves production of an immunogenic agent in the host cell; (c) isolating the immunogenic agent from the host cell; wherein the large scale host cell culture is at least 1 Liter in size, and wherein the host cell is contacted with at least a first RNA effector molecule by addition of the RNA effector molecule to a culture medium of the large scale host cell culture such that the target gene expression is transiently inhibited.

2. A method for producing an immunogenic agent in a large scale host cell culture, comprising: (a) contacting a host cell in a large scale host cell culture with at least a first RNA effector molecule, a portion of which is complementary to at least one target gene of a host cell; (b) maintaining the host cell culture for a time sufficient to modulate expression of the at least one first target gene, wherein the modulation of expression improves production of an immunogenic agent in the host cell; (c) isolating the immunogenic agent from the host cell; wherein the host cell is contacted with at least a first RNA effector molecule by addition of the RNA effector molecule to a culture medium of the large scale host cell culture multiple times throughout production of the immunogenic agent such that the target gene expression is transiently inhibited.

3. The method of paragraph 1 or 2, wherein the host cell in the large scale host cell culture is contacted with a plurality of RNA effector molecules, wherein the plurality of RNA effector molecules modulate expression of at least one target gene, at least two target genes, or a plurality of target genes.

4. A method for production of an immunogenic agent in a cell, the method comprising: (a) contacting a host cell with a plurality of RNA effector molecules, wherein the two or more RNA effector molecules modulate expression of a plurality of target genes; (b) maintaining the cell for a time sufficient to modulate expression of the plurality of target genes, wherein the modulation of expression improves production of the immunogenic agent in the cell; and (c) isolating the immunogenic agent from the cell, wherein the plurality of target genes comprises at least Bax, Bak, and LDH.

5. The method of paragraph 4, wherein the host cell is contacted with the plurality of RNA effector molecules by addition of the RNA effector molecule to a culture medium of the large scale host cell culture such that the target gene expression is transiently inhibited.

6. The method of paragraphs 1 to 5, wherein the RNA effector molecule, or plurality of RNA effector molecules, comprises a double-stranded ribonucleic acid (dsRNA), wherein said dsRNA comprises at least two sequences that are complementary to each other and wherein a sense strand comprises a first sequence and an antisense strand comprises a second sequence comprising a region of complementarity which is substantially complementary to at least part of a target gene, and wherein said region of complementarity is 10-30 nucleotides in length.

7. The method of any of paragraphs 1 to 6, wherein the contacting step is performed by continuous infusion of the RNA effector molecule, or plurality of RNA effector molecules, into the culture medium used for maintaining the host cell culture to produce the immunogenic agent.

8. The method of any of paragraphs 1 to 7, wherein the modulation of expression is inhibition of expression, and wherein the inhibition is a partial inhibition.

9. The method of paragraph 7, wherein the partial inhibition is no greater than a percent inhibition selected from the group consisting of: 10%, 15%, 20%, 25%, 30%, 35%, 40%, 45%, 50%, 55%, 60%, 65%, 70%, 75%, 80%, and 85%.

10. The method of any of paragraphs 1 to 6 or 8-9, wherein the contacting step is repeated at least once.

11. The method of any of paragraphs 1 to 6 or 8-9, wherein the contacting step is repeated multiple times at a frequency selected from the group consisting of: 6 hr, 12 hr, 24 hr, 36 hr, 48 hr, 72 hr, 84 hr, 96 hr, and 108 hr.

12. The method of any of paragraphs 1 to 11, wherein the modulation of expression is inhibition of expression and wherein the contacting step is repeated multiple times, or continuously infused, to maintain an average percent inhibition of at least 50% for the target gene(s) throughout the production of the immunogenic agent.

13. The method of paragraph 12, wherein the average percent inhibition is selected from the group consisting of at least 50%, at least 60%, at least 70%, at least 80%, at least 90%, at least 95%, at least 99%, or 100%.

14. The method of any of paragraphs 1 to 13, wherein the RNA effector molecule is contacted at a concentration of less than 100 nM.

15. The method of any of paragraphs 1 to 14, wherein the RNA effector molecule is contacted at a concentration of less than 20 nM.

16. The method of any of paragraphs 1 to 15, wherein said contacting a host cell in a large scale host cell culture with a RNA effector molecule is done at least 6 hr, at least 12 hr, at least 18 hr, at least 36 hr, at least 48 hr, at least 60 hr, at least 72 hr, at least 96 hr, or at least 120 hr, or at least 1 week, before isolation of the immunogenic agent or prior to harvesting the supernatant.

17. The method of any of paragraphs 1 to 16, wherein the RNA effector molecule is composition formulated in a lipid formulation.

18. The method of any of paragraphs 1 to 17, wherein the RNA effector molecule is a composition formulated in a non-lipid formulation.

19. The method of any of paragraphs 1 to 18, wherein the RNA effector molecule is not shRNA.

20. The method of any of paragraphs 1 to 19, wherein the RNA effector molecule is siRNA.

21. The method of any of paragraphs 1 to 20, wherein the RNA effector molecule is chemically modified.

22. The method of any of paragraphs 1 to 21, wherein the RNA effector molecule is not chemically modified.

23. The method of any of paragraphs 1 to 22, further comprising monitoring at least one measurable parameter selected from the group consisting of cell density, medium pH, oxygen levels, glucose levels, lactic acid levels, temperature, and protein production.

24. The method of any of paragraphs 2 to 23, wherein each of the plurality of different RNA effector molecules is added simultaneously or at different times.

25. The method of any of paragraphs 2 to 23, wherein each of the plurality of different RNA effector molecules is added at the same or different concentrations.

26. The method of any of paragraphs 2 to 6 or 8 to 25, wherein the plurality of different RNA effector molecules is added at the same or different frequencies.

27. The method of any of paragraphs 1 to 26, further comprising contacting the cell with a second agent.

28. The method of paragraph 27, wherein the second agent is selected from the group consisting of: an antibody, a growth factor, an apoptosis inhibitor, a kinase inhibitor, a phosphatase inhibitor, a protease inhibitor, and a histone demethylating agent.

29. The method of paragraph 28, wherein the kinase inhibitor is selected from the group consisting of: a MAP kinase inhibitor, a CDK inhibitor, and K252a.

30. The method of paragraph 28, wherein the phosphatase inhibitor is selected from the group consisting of: sodium vanadate and okadaic acid.

31. The method of paragraph 28, wherein the histone demethylating agent is 5-azacytidine.

32. The method of any of paragraphs 1 to 31, wherein the immunogenic agent is a polypeptide.

33. The method of any of paragraphs 1 to 31, wherein the immunogenic agent is a virus.

34. The method of paragraph 33, wherein the virus is PCV.

35. The method of any of paragraphs 1 to 34, wherein the cell is contacted with the RNA effector molecule at a phase of cell growth selected from the group consisting of: stationary phase, early log phase, mid-log phase, late-log phase, lag phase, and death phase.

36. The method of any of paragraphs 1 to 35, wherein the at least first RNA effector molecule, or at least one of the plurality of RNA effector molecules, comprises a duplex region.

37. The method of any of paragraphs 1 to 36, wherein the at least first RNA effector molecule, or at least one of the plurality of RNA effector molecules, is 15 to 30 nucleotides in length.

38. The method of any of paragraphs 1 to 37, the at least first RNA effector molecule, or at least one of the plurality of RNA effector molecules, is 17 to 28 nucleotides in length.

39. The method of any one of paragraphs 1 to 38, wherein the at least first RNA effector molecule, or at least one of the plurality of RNA effector molecules, comprises at least one modified nucleotide.

40. The method of any of paragraphs 1 to 39, wherein the cell is a plant cell, a fungal cell, or an animal cell.

41. The method of any of paragraphs 1 to 40, wherein the cell is a mammalian cell.

42. The method of paragraph 41, wherein the mammalian cell is a human cell.

43. The method of paragraph 42, wherein the human cell is an adherent cell selected from the group consisting of: SH-SY5Y cells, IMR32 cells, LANS cells, HeLa cells, MCFlOA cells, 293T cells, and SK-BR3 cells.

44. The method of paragraph 42, wherein the human cell is a primary cell selected from the group consisting of: HuVEC cells, HuASMC cells, HKB-II cells, and hMSC cells.

45. The method of paragraph 42, wherein the human cell is selected from the group consisting of: U293 cells, HEK 293 cells, PERC6® cells, Jurkat cells, HT-29 cells, LNCap.FGC cells, A549 cells, MDA MB453 cells, HepG2 cells, THP-I cells, MCF7 cells, BxPC-3 cells, Capan-1 cells, DU145 cells, and PC-3 cells.

46. The method of paragraph 41, wherein the mammalian cell is a rodent cell selected from the group consisting of: BHK21 cells, BHK(TK−) cells, NS0 cells, Sp2/0 cells, EL4 cells, CHO cells, CHO cell derivatives, NIH/3T3 cells, 3T3-L1 cells, ES-D3 cells, H9c2 cells, C2C12 cells, Madin Darby canine kidney (MDCK) cells and miMCD 3 cells.

47. The method of paragraph 46, wherein the CHO cell derivative is selected from the group consisting of: CHO-K1 cells, CHO-DUKX, CHO-DUKX B1, and CHO-DG44 cells.

48. The method of paragraph 42, wherein the cell is selected from the group consisting of: PERC6 cells, HT-29 cells, LNCaP-FGC cells A549 cells, MDA MB453 cells, HepG2 cells, THP-1 cells, miMCD-3 cells, HEK 293 cells, HeLaS3 cells, MCF7 cells, Cos-7 cells, BxPC-3 cells, DU145 cells, Jurkat cells, PC-3 cells, and Capan-1 cells,

49. The method of paragraph 41, wherein the cell is a rodent cell selected from the group consisting of: BHK21, BHK(TK−), NS0 cells, Sp2/0 cells, U293 cells, EL4 cells, CHO cells, and CHO cell derivatives.

50. The method of any of paragraphs 1 to 49, wherein the cell further comprises a genetic construct encoding the immunogenic agent.

51. The method of any of paragraphs 1 to 50, wherein the cell further comprises a genetic construct encoding a viral receptor.

52. The method of any of paragraphs 1 to 51, wherein the target gene encodes a protein that affects protein glycosylation.

53. The method of any of paragraphs 1 to 52, wherein the target gene encodes the immunogenic agent.

54. The method of any of paragraphs 1 to 53, wherein the at least first RNA effector molecule, or at least one of the plurality of RNA effector molecules, is added at a concentration selected from the group consisting of 0.1 nM, 0.5 nM, 0.75 nM, 1 nM, 2 nM, 5 nM, 10 nM, 20 nM, 30 nM, 40 nM, 50 nM, 75 nM, and 100 nM.

55. The method of any of paragraphs 1 to 53, wherein the at least first RNA effector molecule, or at least one of the plurality of RNA effector molecules, is added at an amount of 50 molecules per cell, 100 molecules/cell, 200 molecules/cell, 300 molecules/cell, 400 molecules/cell, 500 molecules/cell, 600 molecules/cell, 700 molecules/cell, 800 molecules/cell, 900 molecules/cell, 1000 molecules/cell, 2000 molecules/cell, or 5000 molecules/cell.

56. The method of any of paragraphs 1 to 53, wherein the at least first RNA effector molecule, or at least one of the plurality of RNA effector molecules, is added at a concentration selected from the group consisting of: 0.01 fmol/106 cells, 0.1 fmol/106 cells, 0.5 fmol/106 cells, 0.75 fmol/106 cells, 1 fmol/106 cells, 2 fmol/106 cells, 5 fmol/106 cells, 10 fmol/106 cells, 20 fmol/106 cells, 30 fmol/106 cells, 40 fmol/106 cells, 50 fmol/106 cells, 60 fmol/106 cells, 100 fmol/106 cells, 200 fmol/106 cells, 300 fmol/106 cells, 400 fmol/106 cells, 500 fmol/106 cells, 700 fmol/106 cells, 800 fmol/106 cells, 900 fmol/106 cells, and 1 pmol/106 cells.

57. The method of any of paragraphs 1 to 56, wherein the at least first RNA effector molecule, or at least one of the plurality of RNA effector molecules, is selected from the group consisting of siRNA, miRNA, dsRNA, saRNA, shRNA, piRNA, tkRNAi, eiRNA, pdRNA, a gapmer, an antagomir, a ribozyme, and any combination thereof.

58. The method of any of paragraphs 1 to 57, wherein the method further comprises contacting the cell with at least one additional RNA effector molecule, or agent, that modulates a cellular process selected from the group consisting of: carbon metabolism and transport, apoptosis, RNAi uptake and/or efficiency, reactive oxygen species production, control of cell cycle, protein folding, protein pyroglutamation, protein deamidation, protein glycosylation, disulfide bond formation, protein secretion, gene amplification, viral replication, viral infection, viral particle release, control of cellular pH, and protein production.

59. The method of any of paragraphs 1 to 3, or 6 to 58, wherein the at least one target gene, is selected from the group consisting of: GLUT1, GLUT2, GLUT3, GLUT4, phosphatidylinositol-3,4,5-trisphosphate 3-phosphatase (PTEN), and lactate dehydrogenase (LDH), and wherein the modulation of expression improves production of a immunogenic agent in the cell by modulating carbon metabolism or transport in the cell.

60. The method of any of paragraphs 1 to 3, or 6 to 58, wherein the at least one target gene is lactate dehydrogenase (LDH) and the RNA effector molecule comprises a sequence selected from SEQ ID NOs:3152540-3152603.

61. The method of any of paragraphs 1 to 3, or 6 to 58, wherein the at least one target gene selected from the group consisting of: Bcl-G, Bax, Bak, Bok, Bad, Bid, Bik, Blk, Hrk, BNIP3, PUMA, NOXA, BimL, Bcl-2, Bcl-xL, Bcl-B, Bcl-w, Boo, Mcl-1, CASP2, CASP3, CASP6, CASP7, CASP8, CASP9, and CASP10; and wherein the modulation of expression improves production of the immunogenic agent in the cell by modulating apoptosis of the cell.

62. The method of claim any of paragraphs 1 to 3, or 6 to 58, wherein the at least one target gene is Bak and the RNA effector molecule comprises a sequence selected from SEQ ID NOs:3152412-3152475.

63. The method of any of paragraphs 1 to 3, or 6 to 58, wherein the at least one target gene is Bax and the RNA effector molecule comprises a sequence selected from SEQ ID NOs:3152476-3152539.

64. The method of paragraph 16 or 17, wherein the RNA effector molecule significantly decreases the fraction of cells that enter early apoptosis.

65. The method of paragraph 3, wherein the plurality of target genes are at least Bax and Bak.

66. The method of paragraph 3, wherein the plurality of target genes are at least Bax, Bac, and LDH.

67. The method of any of paragraphs 4, 5, 65, or 66, wherein the RNA effector molecule, a portion of which is complementary to Bax comprises a sequence selected from SEQ ID NOs:3152476-3152539, wherein the RNA effector molecule, a portion of which is complementary to Bak, comprises a sequence selected from SEQ ID NOs:3152412-3152475.

68. The method of paragraph 4 or 66, wherein the RNA effector molecule, a portion of which is complementary to LDH, comprises a sequence selected from SEQ ID NOs:3152540-3152603

69. The method of any of paragraphs 1 to 3, or 6 to 58, wherein the expression of at least two target genes is modulated and the at least two target genes are selected from the group consisting of: Bcl-G, Bax, Bak, Bok, Bad, Bid, Bik, Blk, Hrk, BNIP3, PUMA, NOXA, and BimL.

70. The method of claim any of paragraphs 1 to 3, 6 to 58, further comprising contacting the cell with a RNA effector molecule comprising a sequence complementary to lactate dehydrogenase (LDH).

71. The method of any of paragraphs 1 to 3, or 6 to 58, wherein the at least one target gene selected from the group consisting of: Ago1, Ago2, Ago3, Ago4, HIWI1, HIWI2, HIWI3, HILI, interferon receptor, ApoE, Eri1 and mannose/GalNAc-receptor, and wherein the modulation of expression improves production of the immunogenic agent in the cell by modulating RNAi uptake and/or efficacy in the cell.

72. The method of any of paragraphs 1 to 3, or 6 to 58, wherein the at least one target gene is selected from the group consisting of NAD(p)H oxidase, peroxidase, constitutive neuronal nitric oxide synthase (cnNOS), myeloperoxidase (MPO), xanthine oxidase (XO), 15-lipoxygenase-1, NADPH cytochrome c2 reductase, NAPH cytochrome c reductase, NADH cytochrome b5 reductase, and cytochrome P4502E1, and wherein the modulation of expression improves production of the immunogenic agent in the cell by inhibiting production of reactive oxygen species in the cell.

73. The method of any of paragraphs 1 to 3, or 6 to 58, wherein the at least one target gene is selected from the group consisting of: MuLV protein, MVM protein, Reo-3 protein, PRV protein, and vesivirus protein; and wherein the modulation of expression improves production of the immunogenic agent in the cell by inhibiting viral infection of the cell.

74. The method of any of paragraphs 1 to 3, or 6 to 58, wherein the at least one target gene is xylose transferase.

75. The method of paragraph 73, wherein the at least one target gene is a vesivirus protein and the at least one RNA effector molecule comprises at least one strand that comprises at least 16 contiguous nucleotides of an oligonucleotide having a sequence selected from SEQ ID NOs:3152604-3152713.

76. The method of any of paragraphs 1 to 3, or 6 to 58, wherein the at least one target gene is selected from the group consisting of: CCNA1, CCNA2, CCNB1, CCNB2, CCNB3, CCND1, CCND2, CCND3, CCNE1, CCNE2, cyclin B, cyclin D, cyclin E, CDK2, CDK4, P10, P21, P27, p53, P57, p161NK4a, P14ARF, and CDK4, and wherein the modulation of expression improves production of the immunogenic agent in the cell by modulating the cell cycle of the cell.

77. The method of any of paragraphs 1 to 3, or 6 to 58, wherein the at least one target gene is selected from the group consisting of: IRE1, PERK, ATF4, ATF6, eIF2alpha, GRP78, GRP94, Bip, Hsp40, HSP47, HSP60, Hsp70, HSP90, HSP100, protein disulfide isomerase, peptidyl prolyl isomerase, calreticulin, calnexin, Erp57, and BAG-1; and wherein the modulation of expression improves production of the protein in the cell by enhancing folding of the protein.

78. The method of any of paragraphs 1 to 3, or 6 to 58, wherein the at least one target gene is a methionine sulfoxide reductase gene in the host cell, and wherein the modulation of expression improves production of the protein in the cell by inhibiting modification of the protein by methionine oxidation.

79. The method of any of paragraphs 1 to 3, or 6 to 58, wherein the target gene is a glutaminyl cyclase gene in the host cell, and wherein the modulation of expression improves production of the protein in the cell by inhibiting modification of the protein by pyroglutamation.

80. The method of any of paragraphs 1 to 3, or 6 to 58, wherein the at least one target gene is selected from the group consisting of: asparagine deamidase and glutamine deamidase; and wherein the modulation of expression improves production of the protein in the cell by inhibiting modification of the protein by deamidation.

81. The method of any of paragraphs 1 to 3, or 6 to 58, wherein the at least one target gene is selected from the group consisting of dolichyl-diphosphooligosaccharide-protein glycosyltransferase, UDP glycosyltransferase, UDP-Gal:βGlcNAcβ1,4-galactosyltransferase, UDP-galactose-ceramide galactosyltransferase, fucosyltransferase, protein O-fucosyltransferase, N-acetylgalactosaminytransferase T-4, O-GlcNAc transferase, oligosaccharyl transferase, O-linked N-acetylglucosamine transferase, α-galactosidase, and β-galactosidase; and wherein the modulation of expression improves production of the protein in the cell by modulating glycosylation of the protein.

82. The method of any of paragraphs 1 to 3, or 6 to 58, wherein the at least one target gene is selected from the group consisting of protein disulfide isomerase and sulfhydryl oxidase; and wherein the modulation of expression improves production of the protein in the cell by modulating disulfide bond formation in the protein.

83. The method of any of paragraphs 1 to 3, or 6 to 58, wherein the at least one target gene is selected from the group consisting of gamma-secretase, p115, a signal recognition particle (SRP) protein, secretin, and a kinase; and wherein the modulation of expression improves production of the protein in the cell by modulating secretion of the protein.

84. The method of any of paragraphs 1 to 3, or 6 to 58, wherein the at least one target gene is a dehydrofolate reductase gene in the host cell, wherein the modulation of expression improves production of the protein in the cell by enhancing gene amplification in the cell.

85. The method of any of paragraphs 1 to 3, or 6 to 58, wherein the at least one target gene is a gene of a virus or a target gene of a cell, thereby producing an immunogenic agent from a host cell having a reduced viral load.

86. The method of paragraph 85, wherein said virus is selected from the group consisting of: vesivirus, MMV, MuLV, PRV, and Reo-3.

87. The method of paragraph 85, wherein said at least one target gene encodes a viral protein.

88. The method of paragraph 85, wherein said at least one target gene encodes a non-viral protein.

89. The method of any of paragraphs 1 to 3, or 6 to 58, wherein the at least one target gene is selected from the group consisting of: pro-oxidant enzymes, BIK, BAD, BIM, HRK, BCLG, HR, NOXA, PUMA, BOK, BOO, BCLB, CASP2, CASP3, CASP6, CASP7, CASP8, CASP9, CASP10, BAX, BAK, BCL2, p53, APAFI, and HSP70; and wherein the modulation of expression improves production of the immunogenic agent in the cell by enhancing the viability of the cell.

90. The method of any of paragraphs 1 to 3, or 6 to 58, wherein the at least one target gene is selected from the group consisting of: CCNA1, CCNA2, CCNB1, CCNB2, CCNB3, CCND1, CCND2, CCND3, CCNE1, CCNE2, cyclin B, cyclin D, cyclin E, CDK2, CDK4, P10, P21, P27, p53, P57, p161NK4a, P14ARF, CDK4, Bcl-G, Bax, Bak, Bok, Bad, Bid, Bik, Blk, Hrk, BNIP3, PUMA, NOXA, BimL, Bcl-2, Bcl-xL, Bcl-B, Bcl-w, Boo, Mcl-1, A1, CASP2, CASP3, CASP6, CASP7, CASP8, CASP9, CASP10, GLUT1, GLUT2, GLUT3, GLUT4, phosphatidylinositol-3,4,5-trisphosphate 3-phosphatase (PTEN), and lactate dehydrogenase (LDH); and wherein the modulation of expression improves production of the immunogenic agent in the cell by enhancing the specific productivity of the cell.

91. The method of any of paragraphs 1 to 3, or 6 to 58, wherein the at least one target gene is selected from the group consisting of: GLUT1, GLUT2, GLUT3, GLUT4, phosphatidylinositol-3,4,5-trisphosphate 3-phosphatase (PTEN), lactate dehydrogenase (LDH), CCNA1, CCNA2, CCNB1, CCNB2, CCNB3, CCND1, CCND2, CCND3, CCNE1, CCNE2, cyclin B, cyclin D, cyclin E, CDK2, CDK4, P10, P21, P27, p53, P57, p161NK4a, P14ARF, and CDK4; wherein the modulation of expression improves production of the immunogenic agent in the cell by modulating nutrient requirements of the cell.

92. The method of any of paragraphs 1 to 3, or 6 to 58, wherein the at least one target gene is selected from the group consisting of: lactate dehydrogenase and lysosomal V-type ATPase; and wherein the modulation of expression improves production of the immunogenic agent in the cell by modulating the pH of the cell.

93. The method of any of paragraphs 1 to 3, or 6 to 58, wherein the at least one target gene is selected from the group consisting of: cytoplasmic actin capping protein (CapZ), Ezrin (VIL2), Laminin A, and Cofilin (CFL1); and wherein the modulation of gene expression improves production of the immunogenic agent in the cell by modulating actin dynamics of the cell

94. The method of paragraph 93, wherein at least one RNA effector molecule inhibits expression of the target gene Cofilin.

95. The method of paragraph 93, wherein at least one RNA effector molecule increases expression of a target gene selected from the group consisting of: cytoplasmic actin capping protein (CapZ), Ezrin (VIL2), and Laminin A.

96. The method of any of paragraphs 1 to 3, or 6 to 58, wherein the at least one target gene is a gene of a host cell latent virus, an adventitious virus, a host cell endogenous retrovirus, or a host cell binding-ligand of such virus.

97. The method of paragraph 96, wherein the target gene is a gene of an endogenous retrovirus (ERV) selected from HERV-K, pt01-Chr10r-17119458, pt01-Chr5-53871501, BaEV, GaLV, HERV-T, ERV-3, HERV-E, HERV-ADP, HERV-I, MER4like, HERV-FRD, HERV-W, HERVH-RTVLH2, HERVH-RGH2, HERV-Hconsensus, HERV-Fcl, hg15-chr3-152465283, HERVL66, HSRV, HFV, HERV-S, HERV-L, HERVL40, HERVL74, HTLV-1, HTLV-2, HIV-1, HIV-2, MPMV, MMTV, HML1, HML2, HML3, HML4, HML7, HML8, HML5, HML10, HML6, HML9, MMTV, FLV, PERV, BLV, EIAV, JSRV, gg01-chr7-7163462, gg01-chrU-52190725, gg01-Chr4-48130894, ALV, gg01-chr1-15168845, gg01-chr4-77338201, gg01-ChrU-163504869, gg01-chr7-5733782, Python-molurus, WDSV, SnRV, Xen1, Gypsy, and Tyl.

98. The method of paragraph 96, wherein the target gene is a gene of a latent virus selected from the group consisting of C serotype adenovirus, avian adenovirus, avian adenovirus-associated virus, human herpesvirus-4 (EBV), and circovirus.

99. The method of paragraph 98, wherein the latent virus is a circovirus, and the target gene is the rep gene of porcine circovirus type 1 (PCV1) or circovirus type 2 (PCV2).

100. The method of paragraph 98, wherein the latent virus is EBV and the target gene is latent membrane protein (LMP)-2A.

101. The method of paragraph 96, wherein the target gene is a gene of an adventitious virus selected from the group consisting of: exogenous retrovirus, human immunodeficiency virus type 1 (HIV-1), HIV-2, human T-cell lymphotropic virus type I (HTLV-I), HTLV-II, human hepatitis A (HHA), HHB, HHC, human cytomegalovirus, EBV, herpesvirus, human herpesvirus 6 (HHV6), HHV7, HHV8, human parvovirus B19, reovirus, polyoma (JC/BK) virus, SV40, human coronavirus, papillomavirus, human papillomavirus, influenza A, B, and C viruses, human enterovirus, human parainfluenza virus, human respiratory syncytial virus, vesivirus, porcine circovirus, lymphocytic choriomeningitis virus (LCMV), lactate dehydrogenase virus, porcine parvovirus, adeno-associated virus, reovirus, rabies virus, leporipoxviruse, avian leukosis virus (ALV), hantaan virus, Marburg virus, SV20, Semliki Forest virus, feline sarcoma virus, porcine parvovirus, mouse hepatitis virus (MHV), murine leukemia virus (MuLV), pneumonia virus of mice (PVM), Theiler's encephalomyelitis virus, murine minute virus, mouse adenovirus (MAV); mouse cytomegalovirus, mouse rotavirus (EDIM), Kilham rat virus, Toolan's H-1 virus, Sendai virus, rat coronavirus, pseudorabies virus, Cache Valley virus, bovine viral diarrhoea virus, bovine parainfluenza virus type 3, bovine respiratory syncytial virus, bovine adenovirus, bovine parvovirus, infectious bovine rhinotracheitis virus, bovine herpesvirus, bovine reovirus, bluetongue virus, bovine polyoma virus, bovine circovirus, vaccinia, orthopoxviruses other than vaccinia, pseudocowpox virus, and leporipoxvirus.

102. The method of paragraph 96, wherein target gene is a host cell binding ligand for an endogenous virus, a latent virus, or an adventitious virus.

103. The method of paragraph 102, wherein the target gene is SLC35A1, Gne, Cmas, B4GalT1, or B4GalT6.

104. The method of any of paragraphs 1 to 3, or 6 to 58, wherein the at least one target gene is selected from the group consisting of FUT8, TSTA3, and GMDS; and wherein the modulation of expression improves production of the immunogenic agent in the cell by modulating fucosylation.

105. The method of paragraph 104, further comprising contacting a host cell with at least one RNA effector molecule that targets a gene that encodes a sialytransferase.

106. The method of paragraph 105, wherein the sialytransferase is selected from the group consisting of ST3 β-galactoside α-2,3-sialyltransferase 1, ST3 β-galactoside α-2,3-sialyltransferase 4, ST3 β-galactoside α-2,3-sialyltransferase 3, ST3 β-galactoside α-2,3-sialyltransferase 5, ST6 (α-N-acetyl-neuraminyl-2,3-β-galactosyl-1,3)-N-acetylgalactosaminide α-2,6-sialyltransferase 6, and ST3 β-galactoside α-2,3-sialyltransferase 2.

107. The method of any of paragraphs 1 to 3, or 6 to 58, wherein the at least one target gene is selected from the group consisting of glutaminase and glutamine dehydrogenase; and wherein the modulation of expression improves production of the immunogenic agent in the cell by modulating ammonia buildup.

108. The method of any of paragraphs 1 to 108, further comprising contacting the host cell with at least one RNA effector molecule that modulates expression of glutaminase.

109. The method of any of paragraphs 1 to 108, further comprising contacting the host cell with at least one RNA effector molecule that modulates expression of glutamine synthetase.

110. A composition comprising: at least one RNA effector molecule, a portion of which is complementary to at least one target gene of a host cell, and a cell medium suitable for culturing the host cell, wherein the RNA effector molecule is capable of modulating expression of the target gene and the modulation of expression enhances production of an immunogenic agent, wherein the at least one RNA effector molecule is an siRNA that comprises an antisense strand comprising at least 16 contiguous nucleotides of the nucleotide sequence selected from the group consisting of SEQ ID NOs:9772-3152339 and SEQ ID NOs:3161121-3176783.

111. The composition of paragraph 110, comprising two or more RNA effector molecules, wherein the two or more RNA effector molecules are each complementary to different target genes.

112. A composition comprising: a plurality of RNA effector molecules, wherein a portion of each RNA effector molecule is complementary to at least one target gene of a host cell, and wherein the composition is capable of modulating expression of Bax, Bak, and LDH, and the modulation of expression enhances production of an immunogenic agent.

113. The composition of paragraph 110 or 112, further comprising at least one additional RNA effector molecule or agent

114. The composition of paragraph 110 or 112, wherein the at least one RNA effector molecule is siRNA.

115. The composition of paragraph 110 or 112, wherein the at least one RNA effector molecule comprises a duplex region.

116. The composition of paragraph 110 or 112, wherein the at least one RNA effector molecule is 15-30 nucleotides in length.

117. The composition of paragraph 110 or 112, wherein the at least one RNA effector molecule is 17-28 nucleotides in length.

118. The composition of paragraph 110 or 112, wherein the at least one RNA effector molecule comprises a modified nucleotide.

119. The composition of paragraph 110, wherein the cell medium is a serum-free medium.

120. The composition of any of paragraphs 110 to 119, wherein the composition is formulated in a non-lipid formulation.

121. The composition of any of paragraphs 110 to 119, wherein the composition is formulated in a lipid formulation.

122. The composition of paragraph 121, wherein the lipid in the formulation comprises a cationic or non-ionic lipid.

123. The composition of any of paragraphs 110 to 122, wherein the composition further comprises one or more cell culture media supplements.

124. The composition of any of paragraphs 110 to 123, wherein the at least one RNA effector molecule comprises a double-stranded ribonucleic acid (dsRNA), wherein said dsRNA comprises at least two sequences that are complementary to each other and wherein a sense strand comprises a first sequence and an antisense strand comprises a second sequence comprising a region of complementarity which is substantially complementary to at least part of a target gene, and wherein said region of complementarity is 10 to 30 nucleotides in length.

125. A kit for enhancing production of an immunogenic agent by a cultured cell, comprising: (a) a substrate comprising one or more assay surfaces suitable for culturing the cell under conditions in which the immunogenic agent is produced; (b) one or more RNA effector molecules, wherein at least a portion of each RNA effector molecule is complementary to a target gene; and (c) a reagent for detecting the immunogenic agent or production thereof by the cell, wherein the one or more RNA effector molecules is an siRNA comprising an antisense strand that comprises at least 16 contiguous nucleotides of the nucleotide sequence selected from the group consisting of: SEQ ID NOs:9772-3152339 and SEQ ID NOs:3161121-3176783.

126. The kit of paragraph 125, wherein the one or more assay surfaces further comprises a matrix for supporting the growth and maintenance of host cells.

127. The kit of paragraph 125, wherein the one or more RNA effector molecules are deposited on the substrate.

128. The kit of paragraph 125, further comprising a carrier for promoting uptake of the RNA effector molecules by the host cell.

129. The kit of paragraph 128, wherein the carrier comprises a cationic lipid composition.

130. The kit of paragraph 128, wherein the carrier is deposited on the substrate.

131. The kit of paragraph 125, further comprising cell culture media suitable for culturing the host cell.

132. The kit of paragraph 125, further comprising instructions for culturing a host cell in the presence of one or more RNA effector molecules and assaying the cell for production of the immunogenic agent.

133. A kit for optimizing production of an immunogenic agent by cultured cells, comprising: (a) a microarray substrate comprising a plurality of assay surfaces, the assay surfaces being suitable for culturing the cells under conditions in which the immunogenic agent is produced; (b) one or more RNA effector molecules, wherein at least a portion of each RNA effector molecule is complementary to a target gene; and (c) a reagent for detecting the effect of the one or more RNA effector molecules on production of the immunogenic agent, wherein the one or more RNA effector molecules is an siRNA comprising an antisense strand that comprises at least 16 contiguous nucleotides of the nucleotide sequence selected from the group consisting of SEQ ID NOs:9772-3152339 and SEQ ID NOs:3161121-3176783.

134. The kit of paragraph 133, wherein the substrate is a multi-well plate or biochip.

135. The kit of paragraph 133, wherein the substrate is a two-dimensional microarray plate or biochip.

136. The kit of paragraph 133, wherein the one or more RNA effector molecules are deposited on the assay surfaces of the substrate.

137. The kit of paragraph 135, wherein a plurality of different RNA effector molecules are deposited on assay surfaces across a first dimension of the microarray.

138. The kit of paragraph 137, wherein the plurality of RNA effector molecules are each complementary to a different target gene.

139. The kit of paragraph wherein the different target genes are Bax, Bak, and LDH.

140. The kit of paragraph 137, wherein a plurality of RNA effector molecules are each complementary to a different region of the same target gene.

141. The kit of paragraph 137, wherein each of the RNA effector molecules comprising the plurality is deposited at varying concentrations on assay surfaces along the second dimension of the microarray.

142. The method of any of paragraphs 1-109, wherein the RNA effector molecule, a portion of which is complementary to the target gene, is a corresponding siRNA that comprises an antisense strand comprising at least 16 contiguous nucleotides of a nucleotide sequence, wherein the nucleotide sequence is set forth in the tables herein.

143. The method of paragraph 121, wherein the lipid formulation comprises a lipid having the following formula:

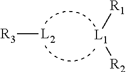

wherein:

R₁ and R₂ are each independently for each occurrence optionally substituted C₁₀-C₃₀ alkyl, optionally substituted C₁₀-C₃₀ alkoxy, optionally substituted C₁₀-C₁₀ alkenyl, optionally substituted C₁₀-C₃₀ alkenyloxy, optionally substituted C₁₀-C₃₀ alkynyl, optionally substituted C₁₀-C₃₀ alkynyloxy, or optionally substituted C₁₀-C₃₀ acyl;

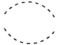

represents a connection between L₂ and L₁ which is:

(1) a single bond between one atom of L₂ and one atom of L₁, wherein
L₁ is C(Rₓ), O, S or N(Q);
L₂ is —CR₅R₆—, —O—, —S—, —N(Q)-, ═C(R₅)—, —C(O)N(Q)-, —C(O)O—, —N(O)C(Q)-, —OC(O)—, or —C(O)—;

(2) a double bond between one atom of L₂ and one atom of L₁; wherein

L₁ is C;
L₂ is —CR₅═, —N(Q)═, —N—, —O—N═, —N(O)—N═, or —C(O)N(Q)—N═;

(3) a single bond between a first atom of L₂ and a first atom of L₁, and a single bond between a second atom of L₂ and the first atom of L₁, wherein
L₁ is C;
L₂ has the formula

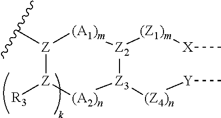

wherein
X is the first atom of L₂, Y is the second atom of L₂, - - - - - represents a single bond to the first atom of L₁, and X and Y are each, independently, selected from the group consisting of —O—, —S—, alkylene, —N(Q)—, —C(O)—, —O(CO)—, —OC(O)N(Q)—, —N(Q)C(O)O—, —C(O)O, —OC(O)O—, —OS(O)(Q₂)O—, and —OP(O)(Q₂)O—;
Z₁ and Z₄ are each, independently, —O—, —S—, —CH₂—, —CHR⁵—, or —CR⁵R⁵—;
Z₂ is CH or N;
Z₃ is CH or N;
or Z₂ and Z₃, taken together, are a single C atom;
Aₜ and A₂ are each, independently, —O—, —S—, —CH₂—, —CHR⁵—, or —CR⁵R⁵—;
each Z is N, C(R₅), or C(R₃);
k is 0, 1, or 2;
each m, independently, is 0 to 5;
each n, independently, is 0 to 5;

where m and n taken together result in a 3, 4, 5, 6, 7 or 8 member ring;

(4) a single bond between a first atom of L₂ and a first atom of L₁, and a single bond between the first atom of L₂ and a second atom of L₁, wherein

(A) L₁ has the formula:

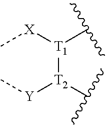

wherein
X is the first atom of L₁, Y is the second atom of L₁, - - - - - represents a single bond to the first atom of L₂, and X and Y are each, independently, selected from the group consisting of —O—, —S—, alkylene, —N(Q)—, —C(O)—, —O(CO)—, —OC(O)N(Q)-, —N(Q)C(O)O—, —C(O)O, —OC(O)O—, —S(O)(Q₂)O—, and —OP(O)(Q₂)O—;
T₁ is CH or N;
T₂ is CH or N;
or T₁ and T₂ taken together are C═C;
L₂ is CR₅; or

(B) L₁ has the formula:

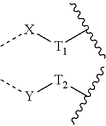

wherein

X is the first atom of L₁, Y is the second atom of L₁, - - - - - represents a single bond to the first atom of L₂, and X and Y are each, independently, selected from the group consisting of —O—, —S—, alkylene, —N(Q)—, —C(O)—, —O(CO)—, —OC(O)N(Q)-, —N(Q)C(O)O—, —C(O)O, —OC(O)O—, —S(O)(Q₂)O—, and —OP(O)(Q₂)O—;
T₁ is —CR₅R₅—, —N(Q)—, —O—, or —S—;
T₂ is —CR₅R₅—, —N(O)—, —O—, or —S—;
L₂ is CR₅ or N;

R₃ has the formula:

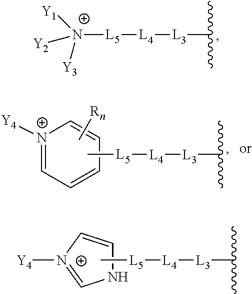

wherein

each of Y₁, Y₂, Y₃, and Y₄, independently, is alkyl, cycloalkyl, aryl, aralkyl, or alkynyl; or

any two of Y₁, Y₂, and Y₃ are taken together with the N atom to which they are attached to form a 3- to 8-member heterocycle; or

Y₁, Y₂, and Y₃ are all be taken together with the N atom to which they are attached to form a bicyclic 5- to 12-member heterocycle;

each Rₙ, independently, is H, halo, cyano, hydroxy, amino, alkyl, alkoxy, cycloalkyl, aryl, heteroaryl, or heterocyclyl;

L₃ is a bond, —N(Q)—, —O—, —S—, —(CR₅R₆)ₐ—, —C(O)—, or a combination of any two of these;

L₄ is a bond, —N(Q)—, —O—, —S—, —(CR₅R₆)ₐ—, —C(O)—, or a combination of any two of these;

L₅ is a bond, —N(Q)—, —O—, —S—, —(CR₅R₆)ₐ—, —C(O)—, or a combination of any two of these;

each occurrence of R₅ and R₆ is, independently, H, halo, cyano, hydroxy, amino, alkyl, alkoxy, cycloalkyl, aryl, heteroaryl, or heterocyclyl; or two R₅ groups on adjacent carbon atoms are taken together to form a double bond between their respective carbon atoms; or two R₅ groups on adjacent carbon atoms and two R₆ groups on the same adjacent carbon atoms are taken together to form a triple bond between their respective carbon atoms;

each a, independently, is 0, 1, 2, or 3;

wherein

an R₅ or R₆ substituent from any of L₃, L₄, or L₅ is optionally taken with an R₅ or R₆ substituent from any of L₃, L₄, or L₅ to form a 3- to 8-member cycloalkyl, heterocyclyl, aryl, or heteroaryl group; and

any one of Y₁, Y₂, or Y₃, is optionally taken together with an R₅ or R₆ group from any of L₃, L₄, and L₅, and atoms to which they are attached, to form a 3- to 8-member heterocyclyl group;

each Q, independently, is H, alkyl, acyl, cycloalkyl, alkenyl, alkynyl, aryl, heteroaryl or heterocyclyl; and

each Q₂, independently, is O, S, N(Q)(Q), alkyl or alkoxy.

### EXAMPLES

### Example 1

### RNA Effector Molecule Synthesis

Where the source of a reagent is not specifically given herein, such reagent can be obtained from any supplier of reagents for molecular biology at a quality/purity standard for application in molecular biology.

Oligonucleotide Synthesis:

All oligonucleotides are synthesized on an AKTAoligopilot synthesizer. Commercially available controlled pore glass solid support (dT-CPG, 500 Å, Prime Synthesis) and RNA phosphoramidites with standard protecting groups, 5′-O-dimethoxytrityl N6-benzoyl-2′-t-butyldimethylsilyl-adenosine-3′-O—N,N′-diisopropyl-2-cyanoethylphosphoramidite, 5′-O-dimethoxytrityl-N4-acetyl-2′-t-butyldimethylsilyl-cytidine-3′-O—N,N′-diisopropyl-2-cyanoethylphosphoramidite, 5′-O-dimethoxytrityl-N2-isobutyl-2′-t-butyldimethylsilyl-guanosine-3′-O—N,N′-diisopropyl-2-cyanoethylphosphoramidite, and 5′-O-dimethoxytrityl-2′-t-butyldimethylsilyl-uridine-3′-O—N,N′-diisopropyl-2-cyanoethylphosphoramidite (Pierce Nucleic Acids Technologies) were used for the oligonucleotide synthesis. The 2′-F phosphoramidites, 5′-O-dimethoxytrityl-N4-acetyl-2′-fluoro-cytidine-3′-O—N,N′-diisopropyl-2-cyanoethyl-phosphoramidite and 5′-O-dimethoxytrityl-2′-fluoro-uridine-3′-O—N,N′-diisopropyl-2-cyanoethyl-phosphoramidite are purchased from (Promega). All phosphoramidites are used at a concentration of 0.2M in acetonitrile (CH₃CN) except for guanosine which is used at 0.2M concentration in 10% THF/ANC (v/v). Coupling/recycling time of 16 min is used. The activator is 5-ethyl thiotetrazole (0.75M, American International Chemicals); for the PO-oxidation iodine/water/pyridine is used and for the PS-oxidation PADS (2%) in 2,6-lutidine/ACN (1:1 v/v) is used.

The 3′-ligand conjugated strands are synthesized using solid support containing the corresponding ligand. For example, the introduction of cholesterol unit in the sequence is performed from a hydroxyprolinol-cholesterol phosphoramidite. Cholesterol is tethered to trans-4-hydroxyprolinol via a 6-aminohexanoate linkage to obtain a hydroxyprolinol-cholesterol moiety. The 5′-end Cy-3 and Cy-5.5 (fluorophore) labeled RNA effector molecules are synthesized from the corresponding Quasar®570 indocarbocyanine Cy™3 phosphoramidite are purchased from Biosearch Technologies (Novato, Calif.). Conjugation of ligands to 5′-end and or internal position is achieved by using appropriately protected ligand-phosphoramidite building block. An extended 15 min coupling of 0.1 M solution of phosphoramidite in anhydrous CH₃CN in the presence of 5-(ethylthio)-1H-tetrazole activator to a solid-support-bound oligonucleotide. Oxidation of the internucleotide phosphite to the phosphate is carried out using standard iodine-water, as reported in the literature, or by treatment with tert-butyl hydroperoxide/acetonitrile/water (10:87:3) with 10 min oxidation wait time conjugated oligonucleotide. Phosphorothioate is introduced by the oxidation of phosphite to phosphorothioate by using a sulfur transfer reagent such as DDTT (purchased from AM Chemicals), PADS and or Beaucage reagent. The cholesterol phosphoramidite is synthesized in house and used at a concentration of 0.1 M in dichloromethane. Coupling time for the cholesterol phosphoramidite is 16 min.

Deprotection I (Nucleobase Deprotection):

After completion of synthesis, the support is transferred to a 100 mL glass bottle (VWR). The oligonucleotide is cleaved from the support with simultaneous deprotection of base and phosphate groups with 80 mL of a mixture of ethanolic ammonia [ammonia:ethanol (3:1)] for 6.5 h at 55° C. The bottle is cooled briefly on ice and then the ethanolic ammonia mixture is filtered into a new 250-mL bottle. The CPG is washed with 2×40 mL portions of ethanol/water (1:1 v/v). The volume of the mixture is then reduced to ˜30 mL by roto-vap. The mixture is then frozen on dry ice and dried under vacuum on a speed vac.

Deprotection II (Removal of 2′-TBDMS Group):

The dried residue is resuspended in 26 mL of triethylamine, triethylamine trihydrofluoride (TEA•3HF) or pyridine-HF and DMSO (3:4:6) and heated at 60° C. for 90 minutes to remove the tert-butyldimethylsilyl (TBDMS) groups at the 2′ position. The reaction is then quenched with 50 mL of 20 mM sodium acetate and the pH is adjusted to 6.5. Oligonucleotide is stored in a freezer until purification.

Analysis:

The oligonucleotides are analyzed by high-performance liquid chromatography (HPLC) prior to purification and selection of buffer and column depends on nature of the sequence and or conjugated ligand.

HPLC Purification:

The ligand-conjugated oligonucleotides are purified by reverse-phase preparative HPLC. The unconjugated oligonucleotides are purified by anion-exchange HPLC on a TSK gel column packed in house. The buffers are 20 mM sodium phosphate (pH 8.5) in 10% CH₃CN (buffer A); and 20 mM sodium phosphate (pH 8.5) in 10% CH₃CN, 1 M NaBr (buffer B). Fractions containing full-length oligonucleotides are pooled, desalted, and lyophilized. Approximately 0.15 OD of desalted oligonucleotides are diluted in water to 150 μL and then pipetted into special vials for CGE and LC/MS analysis. Compounds are then analyzed by LC-ESMS and CGE.

RNA Effector Molecule Preparation:

For the general preparation of RNA effector molecules, equimolar amounts of sense and antisense strand are heated in 1×PBS at 95° C. for 5 min and slowly cooled to room temperature. Integrity of the duplex is confirmed by HPLC analysis.

siRNAs designed to degrade hamster Bax, Bak, and LDH mRNA were synthesized based on publicly available sequence data. A set of approximately 32 siRNAs was designed and synthesized for each target. Each siRNA was added to cell media at 10 nM for 3 days to screen for effect. In a 96 well plate, 29.5 μL of CD CHO media (Gibco) was added to test wells and 47 μL to control wells. To this, 17.5 μL of 100 nM siRNAs in CD CHO media was added to the test wells. To all wells, 3 μL of Lipofectamine™ RNAiMAX transfection reagent (Invitrogen) diluted 1:10 in CD CHO media was added. The mixture was allowed to incubate at room temperature for 15 min and then 125 μL of CD CHO media containing 20,000-30,000 cells was added to all wells. The plates were then placed in a 37° C. CO₂ incubator for 3 days.

After three days, cells were visually inspected for toxicity and then RNA was extracted using a MagMAX™ 96-well RNA extraction kit (Applied Biosys./Ambion®, Austin, Tex.) following manufacturer's instructions. cDNA was made from the RNA using a High Capacity cDNA Reverse Transcription Kit (Applied Biosys.) according to manufacturer's instructions. Finally, qPCR was used to quantify a 25-fold dilution of the target cDNA with a Roche Lightcycler 480 PCR instrument and Roche PCR Probes master mix. Relative knockdown of target genes was calculated using the ΔΔCt method using GAPDH as the internal standard.

For qPCR the following primers and probes were used:

| | | |
|---|---|---|
| Bax | | |
| Forward primer | 5′-GGAGCAGCTCGGAGGCG-3′ | (SEQ ID NO: 3152400) |
| Reverse primer | 5′-AAAAGGCCCCTGTCTTCATGA-3′ | (SEQ ID NO: 3152401) |
| Probe | 5′-6FAM-CGGGCCCACCAGCTCTGAGCA-TAMRA-3′ | (SEQ ID NO: 3152402) |
| Bak | | |
| Forward primer | 5′-CCTCCTAGGCAGGACTGTGA-3′ | (SEQ ID NO: 3152403) |
| Reverse primer | 5′-CCAAGATGCTGTTGGGTTCT-3′ | (SEQ ID NO: 3152404) |
| Probe | 5′-6FAM-TCAGGAACAAGAGACCCAGG-TAMRA-3′ | (SEQ ID NO: 3152405) |
| LDH | | |
| Forward primer | 5′-TCTGTCTGTGGCTGACTTGG-3′ | (SEQ ID NO: 3152406) |
| Reverse primer | 5′-TCACAACATCGGAGATTCCA-3′ | (SEQ ID NO: 3152407) |
| Probe | 5′-6FAM-TGAAGAATCTTAGGCGGGTG-TAMRA-3′ | (SEQ ID NO: 3152408) |
| GAPDH | | |
| Forward primer | 5′-TGGCTACAGCAACAGAGTGG-3′ | (SEQ ID NO: 3152409) |
| Reverse primer | 5′-GTGAGGGAGATGATCGGTGT-3′ | (SEQ ID NO: 3152410) |
| Probe | 5′-VIC-AGTCCCTGTCCAATAACCCC-TAMRA-3′ | (SEQ ID NO: 3152411) |

Following the initial screen at 10 nM, the most potent siRNAs were further tested at concentrations ranging from 100 nM to 1 μM under identical conditions as described above except that the concentrations of siRNAs in the 17.5 μL CD CHO media was modified as needed to obtain the desired final concentration.

An LDH activity assay kit (Cayman Chemical, Ann Arbor, Mich.) was used to test for reduced levels of LDH after 3 to 4 days of treatment with LDH siRNAs. To lyse cells in the 175 μL of media in the 96-well plate wells, 20 μL of 1% TritonX-100 was added and the plates shaken for 10 min at room temperature. The assay was carried out according to manufacturer's protocol.

TABLE 22
dsRNA against hamster Bak1

| start | SEQ | | | SEQ |
|---|---|---|---|---|
| pos. | ID NO | sense (5′-3′) | antisense (5′-3′) | ID NO |
| 89 | | AGGAGGUCUUUCGAAGCUA | UAGCUUCGAAAGACCUCCU | 2260032 |
| 90 | | GGAGGUCUUUCGAAGCUAU | AUAGCUUCGAAAGACCUCC | 2259864 |
| 93 | | GGUCUUUCGAAGCUAUGUU | AACAUAGCUUCGAAAGACC | 2259871 |
| 95 | | UCUUUCGAAGCUAUGUUUU | AAAACAUAGCUUCGAAAGA | |
| 99 | | UCGAAGCUAUGUUUUCCAU | AUGGAAAACAUAGCUUCGA | 2259966 |
| 163 | | AACCCCGAGAUGGACAAUU | AAUUGUCCAUCUCGGGGUU | |
| 185 | | UCCUAGAACCCAACAGCAU | AUGCUGUUGGGUUCUAGGA | |
| 188 | | UAGAACCCAACAGCAUCUU | AAGAUGCUGUUGGGUUCUA | |
| 232 | | AUCAUUGGAGAUGACAUUA | UAAUGUCAUCUCCAAUGAU | |
| 241 | | GAUGACAUUAACCGGAGAU | AUCUCCGGUUAAUGUCAUC | 2260016 |
| 262 | | GACACAGAGUUCCAGAAUU | AAUUCUGGAACUCUGUGUC | |
| 318 | | CGAACUCUUCACCAAGAUU | AAUCUUGGUGAAGAGUUCG | 2259868 |
| 331 | | AAGAUUGCCUCCAGCCUAU | AUAGGCUGGAGGCAAUCUU | 2259985 |
| 333 | | GAUUGCCUCCAGCCUAUUU | AAAUAGGCUGGAGGCAAUC | 2259918 |
| 334 | | AUUGCCUCCAGCCUAUUUA | UAAAUAGGCUGGAGGCAAU | 2259976 |
| 335 | | UUGCCUCCAGCCUAUUUAA | UUAAAUAGGCUGGAGGCAA | 2259895 |
| 415 | | UAUGUCUACCAACGUGGUU | AACCACGUUGGUAGACAUA | 2260083 |
| 476 | | UCAUACUGCACCAUUGCAU | AUGCAAUGGUGCAGUAUGA | |
| 546 | | CAGAGACCCAAUCCUGAUU | AAUCAGGAUUGGGUCUCUG | |
| 551 | | ACCCAAUCCUGAUUGUGAU | AUCACAAUCAGGAUUGGGU | 2259907 |
| 561 | | GAUUGUGAUGACAAUUCUU | AAGAAUUGUCAUCACAAUC | 2259857 |
| 599 | | AGUACGUGGUACACAGAUU | AAUCUGUGUACCACGUACU | 2259943 |
| 607 | | GUACACAGAUUCUUCAGAU | AUCUGAAGAAUCUGUGUAC | |

Exemplary dsRNA sequences against hamster (Cricetulus griseus) Bax are disclosed herein as SEQ ID NOs:3152476-3152539, wherein the even numbered SEQ ID NOs (e.g., NO:3152476) represent the sense strand and the odd numbered SEQ ID NOs (e.g., NO:3152477) represent the complementary antisense strand; in embodiments described herein, the RNA effector molecule can comprise at least 16 contiguous nucleotides of these sequences.

Exemplary dsRNA sequences against hamster (Cricetulus griseus) LDH-A are disclosed herein as SEQ ID NOs:3152540-3152603, wherein the even numbered SEQ ID NOs (e.g., NO:3152540) represent the sense strand and the odd numbered SEQ ID NOs (e.g., NO:3152541) represent the complementary antisense strand; in embodiments described herein, the RNA effector molecule can comprise at least 16 contiguous nucleotides of these sequences.

TABLE 25
Sense and antisense exemplary dsRNA against hamster Bax, Bak, and LDH-A.

| | | | | | |
|---|---|---|---|---|---|
| | SEQ | | | SEQ | IC₅₀ |
| Target | ID NO | Sense (3′ to 5′) | AntiSense (5′ to 3′) | ID NO | (nM) |

Bax Duplex

| | | | | | |
|---|---|---|---|---|---|
| A7 | 3152794 | CCGUCUACCAAGAAGUU | UCAACUUCUUGGUAGAC | 3152795 | 0.38 |
| | | GAdTdT | GGdTdT | | |
| B2 | 3152796 | CAGCUGACAUGUUUGCU | UCAGCAAACAUGUCAGC | 3152797 | 1.46 |
| | | GAdTdT | UGdTdT | | |
| B4 | 3152798 | GUUGUUGCCCUUUUCUA | AGUAGAAAAGGGCAAC | 3152799 | 0.08 |
| | | CUdTdT | AACdTdT | | |
| B11 | 3152800 | GACAGUGACUAUCUUUG | CACAAAGAUAGUCACUG | 3152801 | 0.22 |
| | | UGdTdT | UCdTdT | | |
| C6 | 3152802 | AGCUCUGAGCAGAUCAU | UCAUGAUCUGCUCAGAG | 3152803 | 0.17 |
| | | GAdTdT | CUdTdT | | |

Bak Duplex

| | | | | | |
|---|---|---|---|---|---|
| A2 | 3152804 | GUCUUUCGAAGCUAUGU | AAACAUAGCUUCGAAA | 3152805 | 0.07 |
| | | UUdTdT | GACdTdT | | |
| A10 | 3152806 | GCAGCUUGCUAUCAUUG | UCCAAUGAUAGCAAGCU | 3152807 | 0.38 |
| | | GAdTdT | GCdTdT | | |
| A11 | 3152808 | GCUAUCAUUGGAGAUGA | UGUCAUCUCCAAUGAUA | 3152809 | 0.14 |
| | | CAdTdT | GCdTdT | | |
| B9 | 3152810 | GCCUAUUUAAGAGCGGC | AUGCCGCUCUUAAAUAG | 3152811 | 0.08 |
| | | AUdTdT | GCdTdT | | |
| C7 | 3152812 | CGUGGUACACAGAUUCU | GAAGAAUCUGUGUACC | 3152813 | 0.04 |
| | | UCdTdT | ACGdTdT | | |

LDH Duplex

| | | | | | |
|---|---|---|---|---|---|
| C10 | 3152814 | CUACUUAAGGAAGAACA | UCUGUUCUUCCUUAAGU | 3152815 | 0.06 |
| | | GAdTdT | AGdTdT | | |
| D5 | 3152816 | CAAGCUGGUCAUUGUCA | UGUGACAAUGACCAGCU | 3152817 | 0.06 |
| | | CAdTdT | UGdTdT | | |
| D7 | 3152818 | UCAUCAUUCCCAACGUU | ACAACGUUGGGAAUGA | 3152819 | 0.13 |
| | | GUdTdT | UGAdTdT | | |
| E2 | 3152820 | GAGUGGAGUGAAUGUAG | AGCUACAUUCACUCCAC | 3152821 | 0.40 |
| | | CUdTdT | UCdTdT | | |
| E4 | 3152822 | ACAAGGAGCAGUGGAAU | UCAUUCCACUGCUCCUU | 3152823 | 0.15 |
| | | GAdTdT | GUdTdT | | |

### Example 2

### Enhanced Production of Glucocerebrosidase in Human HT-1080 Cells

The production of human glucocerebrosidase is enhanced in human HT-1080 cells in which the glucocerebrosidase gene has been activated as described in U.S. Pat. No. 5,641,670 (Gene-Activated® GCB (GA-GCB)) by contacting the cells with one or more RNA effector molecules, wherein at least a portion of each RNA effector molecule is complementary to a target gene encoding a host cell mannosidase. The RNA effector molecules inhibits expression of target genes encoding class 1 processing and/or class 2 processing mannosidases, such as Golgi mannosidase IA, Golgi mannosidase IB, Golgi mannosidase IC, and/or Golgi mannosidase II. The coding strand sequences of various mannosidases have been disclosed. See, e.g., Bause, 217 Eur. J. Biochem. 535-40 (1993); Gonzalez et al., 274 J. Biol. Chem. 21375-86 (1999); Misago et al., 92 PNAS 11766-70 (1995); Tremblay et al., 8 Glycobio. 585-95 (1998); Tremblay et al., 275 J. Biol. Chem. 31655-60 (2000). RNA effector molecules targeting the mannosidases can be designed according to the rules of Watson and Crick base pairing and other considerations as disclosed herein, or otherwise known in the art.

Effect of RNA Effector Molecules on GA-GCB Glycoforms:

HT-1080 cells producing GA-GCB are plated and the Production Medium is adjusted to provide RNA effector molecule concentrations ranging from 0 (no drug) to 10 ng/mL. The medium is harvested and the cells are re-fed every 24 hr for 3 days. Samples from the third day are subjected to isoelectric focusing (IEF) analysis to assay the effect of the RNA effector molecules on the expressed glucocerebrosidase. The apparent isoelectric point (pI) of the protein increases in a concentration dependent manner with the concentration of the RNA effector molecules. The RNA effector molecule(s) showing the steepest increase in pI are identified as preferred RNA effector molecules for enhancing production of glucocerebrosidase.

Effect of RNA Effector Molecules on GA-GCB Production:

Ten roller bottles (surface area, 1700 cm2 each) are seeded in Growth Medium (DMEM with 10% calf serum) with HT-1080 cells producing GA-GCB. Following 2 weeks of growth, the medium is aspirated and 200 mL of fresh Production Medium (DMEM/F12, 0% calf serum) is added to three sets of roller bottles. Two sets of four roller bottles are treated with ˜1 ng/mL of the RNA effector molecules. The third group of two roller bottles receives no drug treatment. After about 24 hr, the medium from each roller bottle is harvested and pooled, and a sample is taken for GA-GCB enzymatic activity analysis. The enzyme activity analysis is performed as follows: test article is mixed with the enzyme substrate (4-methylumbelliferyl-β-D-glucopyranoside) and incubated for 1 hr at 37° C. The reaction is stopped by the addition of NaOH/Glycine buffer and fluorescence is quantified by the use of a fluorescence spectrophotometer. Specific activities are expressed as 2,500 Units/mg, where one unit is defined as the conversion of 1 μMole of substrate in 1 hr at 37° C. The entire procedure is repeated for 7 days. Stable production of GA-GCB is observed for all roller bottles throughout the seven daily harvests. Absolute levels of the enzyme, however, may vary according to RNA effector molecule treatment group.

Purification and Characterization of hmGCB:

HmGCB is purified from the culture medium of human fibroblasts grown in the presence of RNA effector molecules. The four N-linked glycans present on hmGCB are released by peptide N-glycosidase F and purified using a Sep-pak C18 cartridge. Oligosaccharides eluting in the 5% acetic acid fraction are permethylated using sodium hydroxide and methyl iodide, dissolved in methanol:water (80:20), and portions of the permethylated glycan mixture are analyzed by matrix-assisted laser desorption ionization time-of-flight mass spectroscopy (MALDI-TOF-MS). The sample is analyzed on a VOYAGER™ STR BIOSPECTROMETRY™ Research Station laser-desorption mass spectrometer (Applied Biosys.) coupled with Delayed Extraction using a matrix of 2,5-dihydroxybenzoic acid. A pattern of pseudomolecular ions is seen in the range m/z 1500-2500, indicating the presence of high-mannose glycans ranging from Man₅GlcNAc₂ to Man₉GlcNAc₂.

The most abundant high mannose glycans present are Man₉GlcNAc₂ and Man₈GlcNAc₂, with decreasing abundances of Man₇GlcNAc₂, Man₆GlcNAc₂, and Man₅GlcNAc₂. A trace amount of a fucosylated biantennary complex glycan containing two sialic acid residues is observed. An approximate indication of the relative abundancy of each glycan is obtained by measuring the peak heights. A more accurate assessment of the average chain length of the high mannose glycans is obtained by MALDI-TOF-MS analysis of the intact glycoprotein. A sharp peak is obtained at about m/z 62,483.1 due to the homogeneity of the glycan chains. The mass of the mature peptide calculated from the amino acid sequence is about 55,577.6, indicating the four N-linked glycan chains contribute 6905.5 to the total mass of hmGCB. From this number, it can be calculated that the average glycan length is 8.15 mannose residues.

Effect of RNA Effector Molecules on GA-GCB Uptake into Macrophages:

GA-GCB produced in HT-1080 cells is used in an in vitro assay to determine uptake efficiency in a mouse macrophage cell line. The specific objective of the experiment is to determine the absolute and mannose receptor-specific uptake of GA-GCB in mouse J774E cells. One day prior to assay, J774E cells are plated at 50,000 cells/cm² in 12-well plates in Growth Medium. For the assay, 0.5 mL of Production Medium (DMEM/F12, 0% calf serum) containing 50 nM vitamin D3 (1, 2-5, Dihydroxy vitamin D3) is added to the cells. Unpurified GA-GCB is added to the test wells at a final concentration of 10 μg/mL in the presence or absence of 2 μg/mL mannan (a competitor for the mannose receptor).

The following forms of GA-GCB are used: GA-GCB from cells treated with a RNA effector molecule (1 μg/mL) and GA-GCB (1 μg/mL) from untreated cells. Control wells receive no GA-GCB. The wells are incubated for 4 hr at 37° C., and then are washed extensively in buffered saline, scraped into GA-GCB enzyme reaction buffer, passed through two freeze/thaw cycles, and clarified by centrifugation. The supernatant is then quantitatively tested for enzyme activity and total protein. Enzyme activity is determined as follows: sample is mixed with the enzyme substrate (4-methylumbelliferyl-β-D-glucopyranoside) and incubated for 1 hr at 37° C. The reaction is stopped by the addition of NaOH/Glycine buffer. Fluorescence is quantified by the use of a fluorescence spectrophotometer. Total protein is determined in freeze/thaw cell lysates by bicinchoninic acid (BCA). Activity is reported as units/mg total protein, where one Unit is defined as the conversion of 1 μMole of substrate in 1 hr at 37° C. Cells treated with a RNA effector molecule will receive the RNA effector molecule in the presence or absence of mannan (2 μg/mL). Internalization of GA-GCB into mouse J744E cells is reported as Units/mg of cell lysates.

The results demonstrate that uptake of GA-GCB from RNA effector molecule treated cells is about 7-fold to 14-fold over background and about 67%-73% inhibitable by mannan. In addition, they also demonstrate that uptake of GA-GCB from untreated cells is about 3-fold over background and 53% inhibitable by mannan. Thus, the inhibition of intracellular mannosidases by RNA effector molecules results in GA-GCB that can be transported into cells efficiently via the mannose receptor. Improvement in targeting of GA-GCB to cells via mannose receptors can therefore be optimized by production of GA-GCB in the presence of one or more RNA effector molecules.

### Example 3

### Growth Curves of Suspended CHO—S Cells Treated with Different siRNAs

Flasks were set up with approximately 400,000 cells/mL in 50 mL of total volume. First, 2.5 μL of 20 μM Invitrogen Stealth FITC-siRNA or 50 μL of 1 μM Bax siRNA and 50 μL of 1 μM Bak siRNA or 50 μL of 1 μM LDH siRNA were added to three different 14.3 mL volumes of CD CHO media (GIBCO). The solutions were gently mixed and then 85.5 μL of LIPOFECTAMINE™ RNAiMAX transfection reagent (Invitrogen) was added to each and the solutions gently mixed again. The solutions were allowed to incubate at room temperature for 15 min. After 15 min, 32.8 mL of warmed media was added to each solution. Finally, 2.9 mL of media with 7,000,000 cells/mL was added and the flasks put on a shaker plate set at 160 rpm in a 37° C. CO₂ incubator. Each following day an aliquot was taken from the media to count cells and determine their viability in a Beckman-Coulter cell counter.

On days 2 and 4, additional siRNAs were added. To do this, 25 mL was removed from each flask and spun at ˜400×g for 5 minutes to pellet the cells. Then, 14.3 mL of the cell-free media was removed to a separate tube and siRNAs and LIPOFECTAMINE™ RNAiMAX reagent were added as above. The solutions were gently mixed and allowed to incubate at room temperature for 15 min. The solutions were added back to their respective cell pellet, mixed with a pipette to break up cell clumps and then introduced back to their original flasks.

### Example 4

### Inhibition of Bax, Bak and LDH Enhances Viability of Cells in Culture

Bax and Bak are members of the mitochondrial-regulating BCL-2 protein family that play pivotal pro-apoptotic (capable of inducing programmed cell death) roles. As described herein, potent siRNAs directed against Bax and Bak with IC₅₀s in the low pico molar range were added at periodic intervals to CHO cells grown in a 1 L bioreactor. In addition, an siRNA directed against lactate dehydrogenase (LDH) was also included in the siRNA formulation. LDH catalyzes the conversion of pyruvate to lactate during times of anaerobic stress. Lactate is a major metabolic waste product produced in cells grown in culture and has been shown to inhibit both cell growth and metabolic pathways. Because the activation of the Bax/Bak and LDH pathways is thought to limit the growth potential of cells in culture, the effect of adding potent siRNAs directed against these genes to CHO cells grown in suspension under 1 L bioprocessing-like conditions was evaluated. When compared to CHO cells treated with a non-specific FITC-labeled siRNA, the Bax/Bak/LDH siRNA-treated cells grew to a cell density that was 90% greater than the control with a corresponding 2-fold decreased apoptotic death rate.

Materials and Methods:

Suspension-adapted CHO cells were obtained from Invitrogen and were grown (0.2×10⁶ cells/mL seed density) in a 1 L disposable bioreactor (Sartorius, Bohemia, N.Y.) at 37° C. and 5.5% CO₂ using DG44 chemically defined media (Invitrogen; #12610-010) with constant stirring at a rate suggested by the manufacturer. Starting on day-4 following seeding, the cell cultures were supplemented with 5% culture volume (30 mL) using CHO CD Efficient Feed media (Invitrogen; 10234, 10240). The cultures were then fed every 48 hr using the same feed media and volume.

Bax, Bak, and LDH siRNA sequences are provided in Table 10 and synthesized initially at small scale without modification (except for 3′ dTdT) by RLD small scale synthesis followed by medium scale synthesis. Control siRNA was purchased from Invitrogen (FITC-labeled oligo; #44-2926). Each siRNA was added to the 1 L bioreactor at a final concentration of 1 nM and formulated for transfection using Lipofectamine RNAiMax transfection reagent (Invitrogen). Bax, Bak, and LDH siRNAs were formulated together for a final combined siRNA concentration of 3 nM. The control siRNA formulation contained 6 mL DG44 media, 240 μL LIPOFECTAMINE™ RNAiMax reagent, and 30 μL FITC-labeled oligo (20 μM stock concentration). The experimental siRNA formulation contained 6 mL DG44 media, 240 μL LIPOFECTAMINE™ RNAiMax reagent, and 6 uL of each Bax, Bak, and LDH siRNA (100 μM stock concentrations). Both control and experimental siRNAs were incubated at room temperature for 15 min prior to addition to the culture media starting on day 0 and dosed again at similar concentrations every 48 hr for a total of six doses. Each day, 5 mL culture samples were removed, the cells counted and viability determined using Trypan blue dye (Sigma Aldrich) exclusion with a hemocytometer. All cell samples were taken before any further addition of siRNA or nutrient feeds. The remaining cells were aliquoted, spun down to form a cell pellet and frozen at −70° C. until needed for the following assays: qPCR, lactate, glucose, LDH, and caspase 3.

TABLE 26
Oligo sense strand sequences of siRNAs

| Target | SEQ ID NO | Strand | IC₅₀ | Sequence 5′ to 3′ |
|---|---|---|---|---|
| LDH C10 | 3152814 | sense | 16 pM | CUACUUAAGGAAGAACAGAdTdT |
| Bak A2 A-54123.1 | 3152804 | sense | 70 pM | GUCUUUCGAAGCUAUGUUUdTdT |
| Bax B4 A-54091.1 | 3152798 | sense | 80 pM | GUUGUUGCCCUUUUCUACUdTdT |

Results:

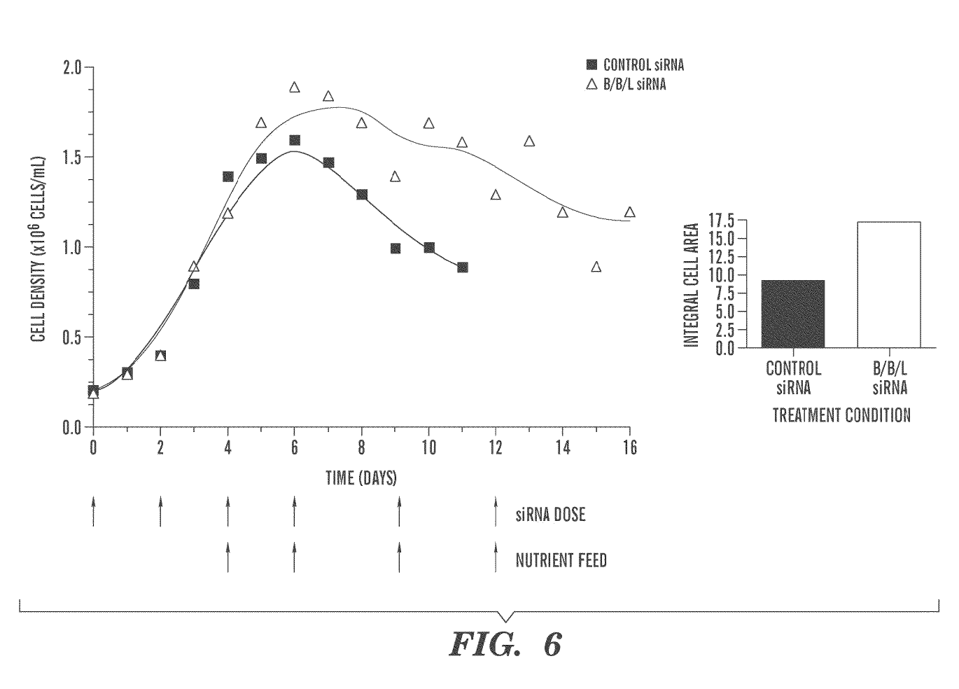

The addition of Bax/Bak/LDH siRNAs to CHO cell cultures improves viable cell density by approximately 2-fold (FIG. 6) when compared to a control transfection using a non-specific FITC-labeled siRNA. The control cell population reached a maximum cell density of ˜1.5×10⁶ cells per mL on day 6; whereas, the Bax/Bak/LDH siRNA-treated cells achieved a maximum cell density of ˜1.8×10⁶ cells per mL on day 7. The integral cell area (IGA) for the Bax/Bak/LDH-treated cells increased ˜90% over the control siRNA-treated cells (FIG. 6, inset).

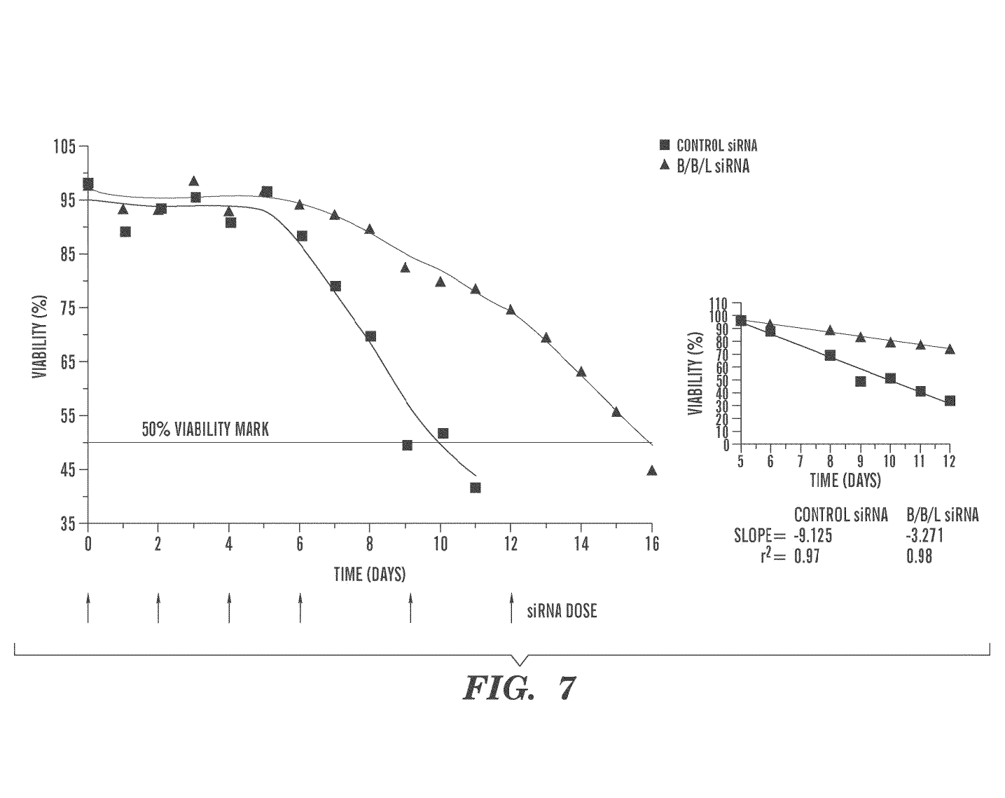

Fifty percent viability of the control cells was observed on day 10 and on day 16 for the Bax/Bak/LDH-treated cells (FIG. 7). Both samples exhibited comparably high viability starting on day-0 until day-5. Cell viability started to decay below 90% starting on day 6 for the control-treated sample and on day 7 for the experimental. Cell death rates are directly proportional to the slope of the percent viability response curve. Sharper slopes indicate faster apoptotic death rates compared to shallower slopes. The rate of apoptotic cell death was 2.8-fold faster for the control compared to the Bax/Bak/LDH siRNA-treated culture (FIG. 7, inset).

These data strongly support the concept that soluble siRNAs when added to CHO cells grown in suspension in a 1 L bioreactor can have a positive effect on both cell density and viability when compared to a non-specific control siRNA.

Both lactate dehydrogenase enzyme activity and lactate levels are decreased in CHO cells following Bax/Bak/LDH siRNA treatment.

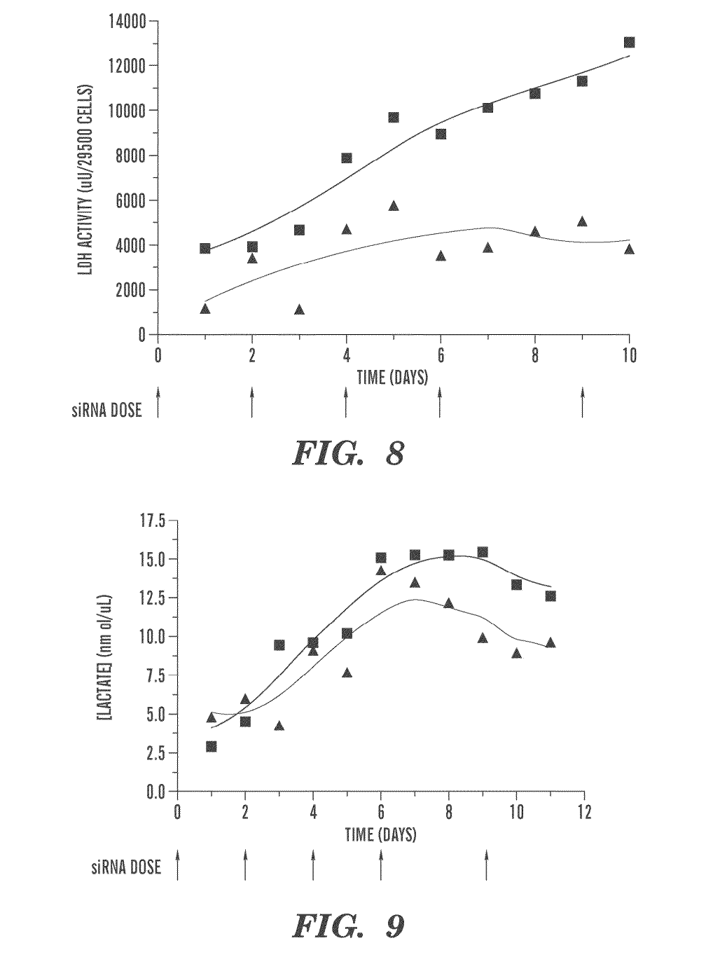

Lactate dehydrogenase enzyme activity was followed during the course of the cell growth curve (FIG. 8). Area under the curve (AUC) analysis indicated a 67% decrease in enzymatic activity in the Bax/Bak/LDH siRNA-treated cells compared to the control siRNA-treated cells. A corresponding decrease in lactate levels was observed (FIG. 9). The observed lactate level decrease in the Bax/Bak/LDH siRNA-treated culture as determined by AUC analysis was approximately 33%, about one-half that observed for the enzyme activity decrease, suggesting the LDH pyruvate to lactate conversion rate increased to compensate for decreased enzyme concentrations.

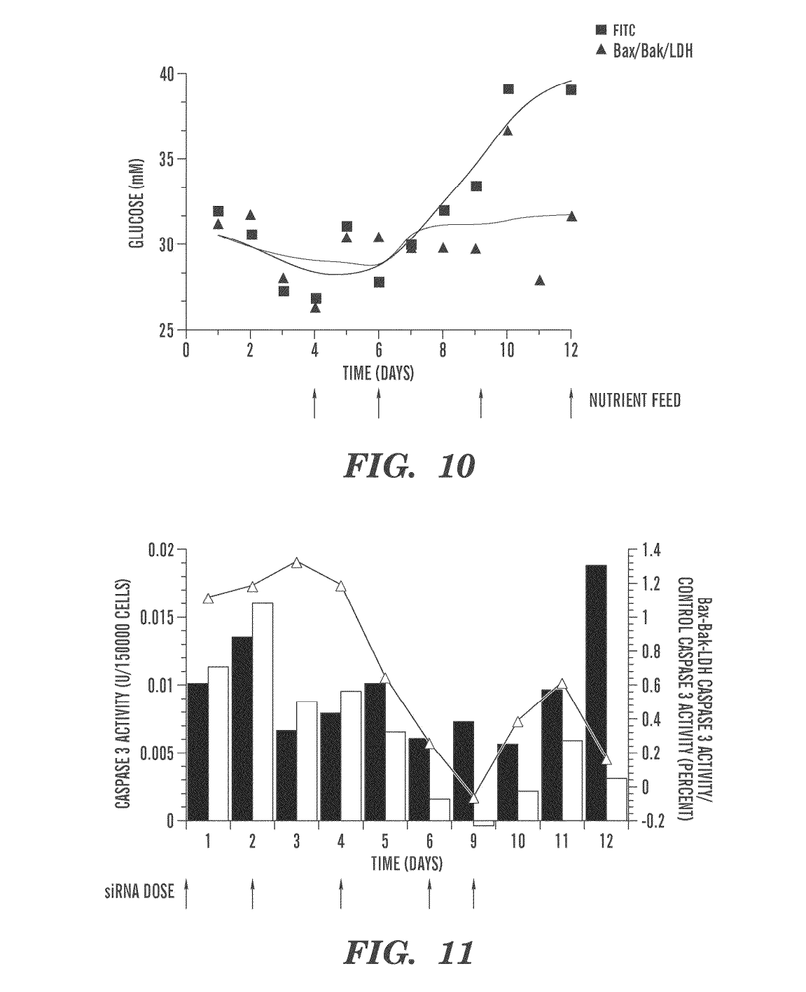

Glucose consumption in control siRNA-treated cells decreases following day 7 of the growth curve. Glucose was used as part of the culture feeding strategy and monitored throughout the growth curve. Prior to day 7, both the control and experimental cultures utilized glucose to the same extent (FIG. 10). After day 7, the Bax/Bak/LDH siRNA-treated cells continued to use glucose as they did prior to day 7 but the control cell population appeared to decrease their glucose consumption.

These data demonstrate that Bax/Bax/LDH siRNAs, when added to 1 L CHO bioprocessing cultures, promote glucose utilization post log phase growth compared to the control siRNA-treated culture that does not suggesting the control cells are dead or incapable of glucose metabolism.

Bax/Bak/LDH siRNAs when added to 1 L CHO bioprocessing cultures significantly decrease Caspase 3 activity compared to the control siRNA. Caspase 3 activation is the penultimate step that leads to DNA degradation in cells undergoing apoptotic death. Since both Bax and Bak proteins are upstream of this process, it is expected that a Bax/Bak knockdown would decrease Caspase 3 activity as well. A biphasic Caspase 3 activity response was observed (FIG. 11) for both the control and experimental conditions. During log phase growth, both the Bax/Bak/LDH-siRNA-treated and control siRNA-treated cell cultures had similar Caspase 3 levels. The reason for active Caspase 3 in non-apoptotic cells is uncertain; but during post log phase, the Bax/Bak/LDH siRNA-treated cell culture had markedly less Caspase 3 activity compared to the control cell population with no Caspase 3 activity observed on day 9 and <10% activity present the experimental cell population on day 12 compared to control.

These data demonstrate the Bax/Bak/LDH siRNAs block the ability of Bax and Bak to activate mitochondrial-induced apoptosis, confirming the appropriate target pathway has been affected.

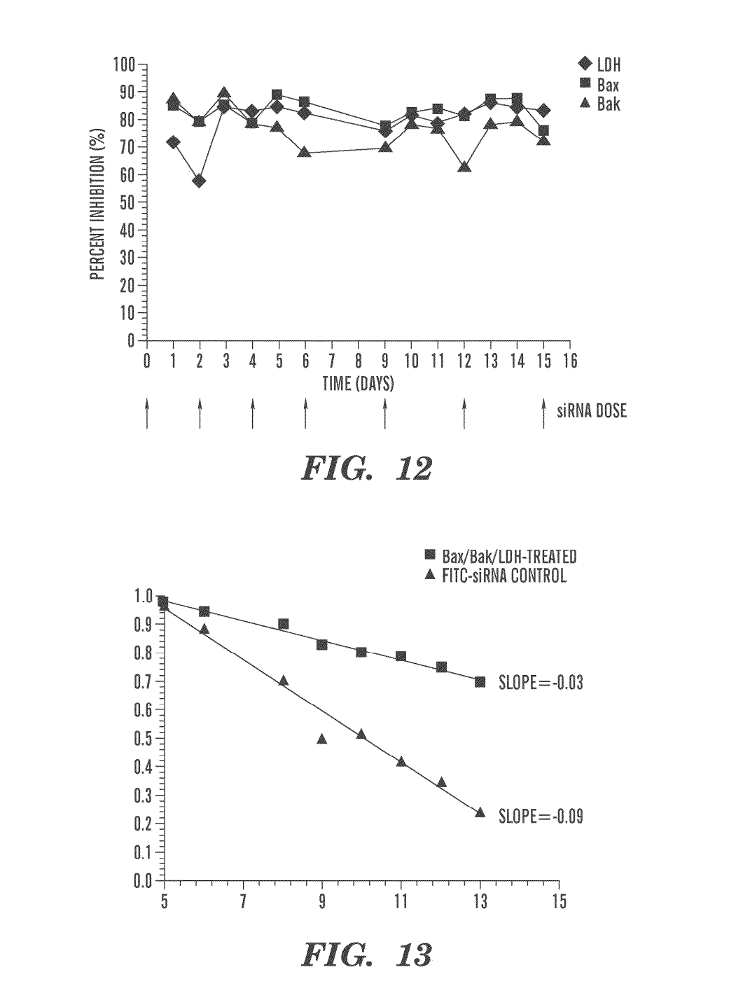

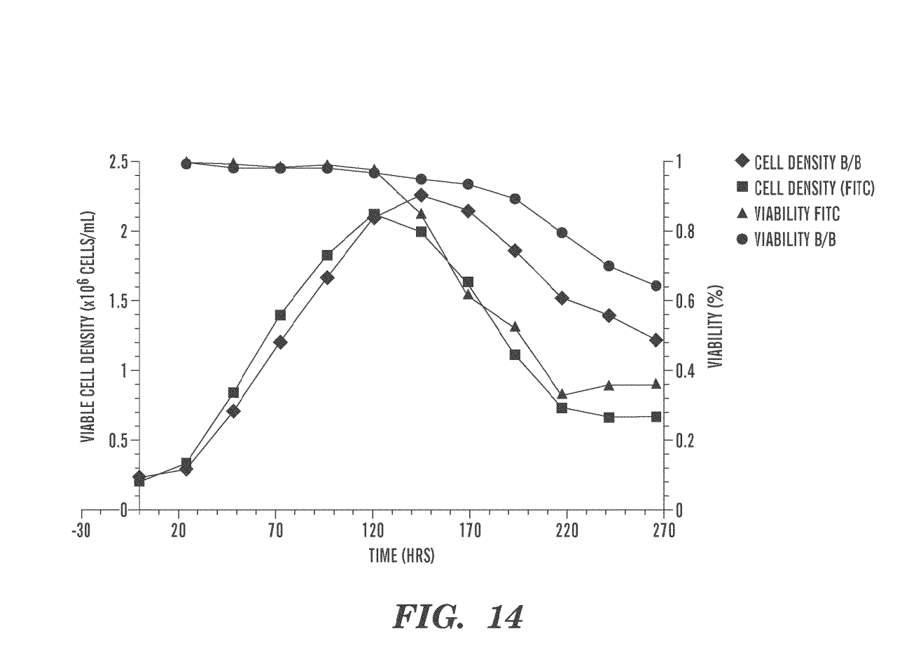

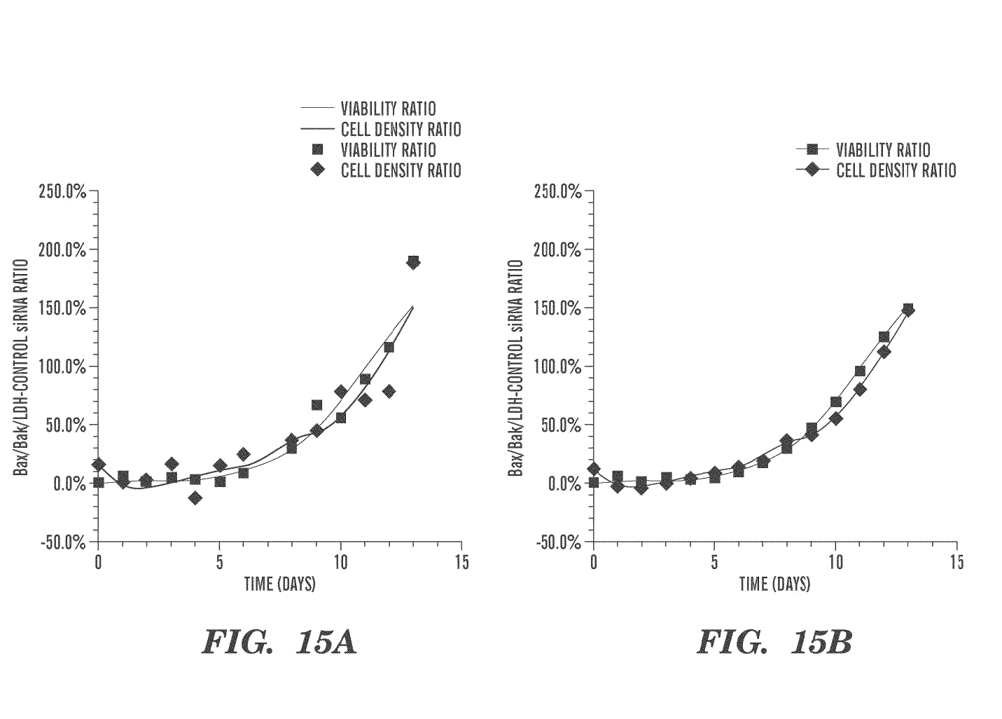

Bax/Bak/LDH siRNAs, when dosed multiple times over a 2-week time course, can maintain >80% mRNA knockdown. A recent publication has reported that both Bax and Bak mRNA should be comparably knocked down to maintain a maximum block of apoptosis (Lim et al., 8 Metabolic Eng. 509-22 (2006)), although another group suggested >80% mRNA knockdown was sufficient for LDH (Kim & Lee, 74 Appl. Microbiol. Biotech. 152-59 (2007)) to reduce LDH activity. Therefore, the aim of multiple siRNA doses was to keep the percent knockdown for all three genes to be >80%. Bax and LDH message knockdown through most of the time course was in fact >80% (FIG. 12). The Bak mRNA knockdown hovered above and below the 80% mark through the time course. This siRNA appeared to benefit most from the multiple doses as suggested by the zigzag response pattern that seems to correlate with each new dose. A zigzag effect is also observed with the other siRNAs, but not as dramatic as the Bak siRNA.

These data demonstrate that all three siRNAs used in this study maintained target mRNA knockdown throughout the two week time course. Even though the message knockdown IC₅₀ for the Bak siRNA was similar to Bax, the mRNA knockdown maintenance during the time course was not comparable. The reason for this is uncertain but suggests that other Bak siRNAs should be evaluated.

Summary:

Silencing RNAs, directed against the apoptotic regulators Bax and Bak, in combination with an siRNA directed against a key metabolic enzyme, lactate dehydrogenase, were evaluated for knockdown activity in Chinese Hamster Ovary cells during a two week time course using a 1 L bioreactor. The results presented herein clearly support the concept that silencing RNAs can be appropriately formulated for efficient uptake into CHO cells grown in suspension under bioprocessing-like conditions. Bax/Bak/LDH siRNAs when dosed multiple times over the two week time course maintained >80% mRNA knockdown which was sufficient to lower both Caspase 3 and LDH activities resulting in increased cell density and viability compared to a non-specific siRNA control. Furthermore, these data demonstrate that multiple siRNAs (at least three) can be added simultaneously with multiple doses in suspension cell cultures with each having its desired knockdown effect and that transfection reagents can be identified that are well tolerated by CHO cells with minimal effect on viability.

### Example 5

### Improved ADCC of Antibodies by Use of RNA Effectors

Many therapeutic antibodies, particularly anticancer therapeutic antibodies, require antibody-dependent cellular cytotoxicity (ADCC) for efficacy. In order to achieve high ADCC, it is believed that proper glycosylation of the antibody is necessary. For example, antibodies lacking the core fucose of the Fc oligosaccharides have been found to exhibit much higher ADCC in humans than their fucosylated counterparts. In addition, extensive α 2,6-sialation of N-linked oligosaccharides in antibodies is also thought to reduce ADCC.

Therefore, it is desirable to produce antibodies with substantially reduced amounts of fucosylation, as well as reduced a 2,6-sialation.

Fucosylation, particularly a 1,6-fucosylation of antibodies is achieved through a number of enzymatic steps, including:

(i) GDP-mannose 4,6 dehydratase (encoded by GMDS), catalyzing the conversion of GDP-mannose to GDP-4-keto-6-deoxymannose;

(ii) GDP-4-keto-6-deoxy-D-mannose epimerase reductase (encoded by TSTA3), which catalyzes the two step epimerase and the reductase reactions in GDP-D-mannose metabolism, converting GDP-4-keto-6-D-deoxymannose to GDP-L-fucose, GDP-L-fucose is the substrate of several fucosyltransferases; and

(iii) Fucosyltransferase 8 (1,6 fucosyltransferase) (encoded by FUT8), which catalyzes the transfer of fucose from GDP-fucose to N-linked type complex glycopeptides.

Cells which are deleted or deficient in the 1,6, fucosyltransferases have been isolated, and are currently used to produce antibodies with reduced fucosylation. However, the cells have a slow doubling time, and require special conditions to grow. Furthermore, the cells are not available in many genetic backgrounds.

High sialation of antibodies has also been suggested to result in reduced ADCC. Sialation occurs through the action of sialyltransferases such as those described herein.

Therefore, increased ADCC of antibodies is achieved by producing the antibody in host cells using the methods described herein. For example, host cells expressing antibodies are contacted with siRNAs directed against any one of:
FUT8: Antisense sequence containing at least 16 contiguous nucleotides from SEQ ID NOs:209841-210227; or siRNAs comprising at least one strand selected from SEQ ID NOs:3152714-3152753, or those described herein;
GMDS: dsRNA comprising an antisense strand comprising at least 16 contiguous nucleotides (e.g., at least 17, at least 18, at least 19 nucleotides) of the oligonucleotide having a nucleotide sequence selected from the group consisting of SEQ ID NOs:1688202-1688519; and SEQ ID NOs:3152754-3152793;
TSTA3: a dsRNA molecule targeting TSTA3 can comprise an antisense strand comprising at least 16 contiguous nucleotides (e.g., at least 17, at least 18, at least 19 nucleotides) of an oligonucleotide molecule selected from the group consisting of SEQ ID NOs:1839578-1839937.

Twelve separate cultures CHO cells expressing a human anti-CD20 antibody are grown in culture flasks, initially seeded on day 1 at a density of ˜200,000 cells/ml, and on day 2 are given the following treatments:
Flask A: Transfection agent only;
Flask B: Transfection agent containing 1 nM (final concentration after addition)

Luciferase dsRNA as negative control;
Flask C: 1 nM FUT8 dsRNA in transfection reagent;
Flask D: 1 nM TSTA3 dsRNA in transfection reagent;
Flask E: 1 nM GMDS dsRNA in transfection reagent;
Flask F: 1 nM TSTA3 dsRNA+1 nM FUT8 dsRNAs in transfection reagent;
Flask G: 1 nM GMDS dsRNA+1 nM FUT8 dsRNAs in transfection reagent;
Flask H: 1 nM TSTA3 dsRNA+1 nM GMDS dsRNAs in transfection reagent;
Flask I: 1 nM St6GalNac6 dsRNA+1 nM FUT8 dsRNAs in transfection reagent;
Flask J: 1 nM St6GalNac6 dsRNA+1 nM GMDS dsRNAs in transfection reagent;
Flask K: 1 nM St6GalNac6 dsRNA+1 nM TSTA3 dsRNAs in transfection reagent;
Flask L: 1 nM St6GalNac6 dsRNA+1 nM FUT8 dsRNAs+1 nM GMDS dsRNA in transfection reagent;

Cells are grown for an additional 4 days, and supernatant of each flask is collected. Antibodies are isolated from the supernatant using protein A-sepharose chromatography. The partially purified antibodies are characterized for overall yield (by ELISA using anti-human Ab), antigen binding (e.g., CD20 binding), and for ADCC (using, for example, the lactate dehydrogenase release assay). The oligosaccharide structure of the antibodies isolated from the different cells are characterized MALDI-TOF mass spectrometry in positive-ion mode.

Exemplary dsRNA sequences against hamster (Cricetulus griseus) fucosyltransferase (FUT8) are disclosed herein as SEQ ID NOs:3152714-3152753, wherein the even numbered SEQ ID NOs (e.g., NO:3152714) represent the sense strand and the odd numbered SEQ ID NOs (e.g., NO:3152715) represent the complementary antisense strand; in embodiments described herein, the RNA effector molecule can comprise at least 16 contiguous nucleotides.

TABLE 43
Screen of FUT8 siRNAs at 1 nM with 1 day incubation on adherent DG44 cells

| SEQ ID NO. | Name | antisense sequence | % mRNA knockdown (1 nM) |
|---|---|---|---|
| 3157117 | AD-25348 | AUGCCCGCAUUUUCAGAGUdTdT | 94.8 |
| 209866 | AD-25349 | UAAUCCAACGCCAGGAACCdTdT | 96.6 |
| 3157118 | AD-25350 | AAAAGAAUGAGCAUAAUCCdTdT | 93.0 |
| 3157119 | AD-25351 | AAAUGACCACCUAUAUAAAdTdT | 28.9 |
| 3157120 | AD-25352 | AACCAAAUGACCACCUAUAdTdT | 83.8 |
| 3157121 | AD-25353 | UAUCUCGAACCAAAUGACCdTdT | 94.3 |
| 209898 | AD-25354 | UUAUCUCGAACCAAAUGACdTdT | 87.9 |
| 3157122 | AD-25357 | AACCAGAGCUCUUUAGCUCdTdT | 93.2 |
| 3157123 | AD-25358 | AUCUGUCAUGAUAGACCUUdTdT | 66.0 |
| 210049 | AD-25359 | UUAUUCUCCGCUGGACCAGdTdT | 80.3 |
| 3157124 | AD-25360 | AUGAGUGUUCGCUGGGUGCdTdT | 72.7 |
| 3157125 | AD-25361 | UUACAGGUCUAAACACAGUdTdT | 74.3 |
| 3157126 | AD-25362 | AACUGGAUGUUUGAAGCCAdTdT | 84.3 |
| 3157127 | AD-25363 | UUGGAGUACUUUGUCUUUGdTdT | 86.6 |
| 3157128 | AD-25364 | AAUUGGAGUACUUUGUCUUdTdT | 77.3 |
| 3125129 | AD-25365 | UAAUUGGAGUACUUUGUCUdTdT | 81.7 |
| 209878 | AD-25366 | AUAAUUGGAGUACUUUGUCdTdT | 77.9 |
| 209904 | AD-25367 | AAGUGUAUAUCCAGGAUCAdTdT | 81.7 |
| 3125130 | AD-25355 | UUGCAAGAAUCUUGGAGAGdTdT | 92.0 |
| 209885 | AD-25356 | AAAACACGGACUCUUCCUGdTdT | 93.3 |

TABLE 44
Dose-response of FUT8 siRNAs in DG44 cells

| | % mRNA knockdown | | | | |
|---|---|---|---|---|---|
| | 100 nM | 10 nM | 1 nM | 100 pM | 10 picom |
| AD-25348 | 97.4 | 93.7 | 83.0 | 47.6 | 24.6 |
| AD-25349 | 99.2 | 97.4 | 87.0 | 74.3 | 22.8 |
| AD-25353 | 96.5 | 97.0 | 89.9 | 57.2 | 67.2 |
| AD-25357 | 94.0 | 91.5 | 55.1 | 51.9 | 14.9 |
| AD-25356 | 96.1 | 95.6 | 92.7 | 75.0 | 26.7 |

### Example 6

### Use of Bax/Bak in High-Glucose Culture

In general, inclusion of high concentrations of glucose (e.g., at least 15 mM) during growth of cells in bioprocessing results in accumulation of lactic acid in the growth media which can be deleterious to cell growth. Lactic acid accumulation results in premature apoptosis. Since providing high levels of a carbon source such as glucose would be otherwise highly advantageous, a method of growing cells in high glucose without triggering lactic acid accumulation and subsequent apoptosis would be highly desirable.

In this example, a RNA effector molecule targeting pro-apoptotic genes are used to allow cells to grow at higher glucose concentrations of at least 10 mM (for example, at least 15 mM, at least 20 mM, at least 25 mM, at least 30 mM or more) in the growth medium without undergoing apoptosis.

On day 0, host cells capable of expressing the immunogenic agent are contacted with 1 nM each of RNA effectors targeting Bax and Bak (optionally also with 1 nM dsRNA targeting LDH) in growth medium containing normal levels (˜4-6 mM) of glucose. Approximately 24 hr afterwards, cells are switched to media containing 15 mM glucose. Subsequently, RNA effectors targeting Bax and Bak are further provided at 1 nM every 3-5 days. Protein production in these cells is compared with those from cells not transfected with RNA effector molecules (or transfected with an unspecific control RNA effector).

Other RNA effectors useful to permit growth in high glucose can include those targeting any pro-apoptotic genes, including those described herein. Other examples include RNA effector molecules comprising an an antisense strand comprising at least 16 contiguous nucleotides (e.g., at least 17, at least 18, at least 19 nucleotides) of an oligonucleotide nucleotide having a sequence selected from the group consisting of the following:

TABLE 30
Caspase

| SEQ | | | Avg | siRNA |
|---|---|---|---|---|
| ID NO: | consL | Descrption | Coverage | SEQ ID NOs: |
| 5854 | 1136 | caspase 1 | 2.306 | 1964106-1964500 |
| 8056 | 612 | caspase 2 | 1.166 | 2718675-2719039 |
| 5746 | 1157 | caspase 3 | 11.813 | 1924836-1925195 |
| 7120 | 855 | caspase 6 | 4.965 | 2408466-2408843 |
| 6798 | 926 | caspase 7 | 0.436 | 2301618-2301960 |
| 8917 | 414 | caspase 8 | 0.2 | 2995593-2995870 |
| 4250 | 1492 | caspase 9 | 1.769 | 1412589-1412860 |
| 5608 | 1188 | caspase 12 | 0.856 | 1875252-1875646 |

### Example 7

### Efficacy of siRNA's in PK15 Cells and in DG44 Cells

siRNA Screening in DG44 Cells:

siRNAs against CHO targets of interest are designed and synthesized. Sets of siRNAs (duplex) to be screened are added to cell media at between 100 μM and 10 nM for between 1 and 4 days for effect. In a 96 well plate, 29.5 μL of CD DG44 media (GIBCO™ Invitrogen) supplemented with 8 mM L-glutamine and 0.18% PLURONIC F68® is added to test wells and 47 μL to control wells. To this, 17.5 μL of siRNA at 10 times the final desired concentration in CD DG44 media is added to the test wells. To all wells, 3 μL of LIPOFECTAMINE® transfection reagent RNAiMAx (Invitrogen) diluted 1:10 in CD DG44 media is added. The mixture is allowed to incubate at room temperature for 15 min and then 125 μL of CD DG44 media containing approximately 20,000 DG44 cells is added to all wells. The plates are then placed in a 37° C. CO₂ incubator for between 1 and 4 days.

After incubation, cells are visually inspected for toxicity and RNA extracted using a MagMax 96-well RNA extraction kit (Ambion, Life Technologies Corp., Carlsbad, Calif.) following the manufacturer's instructions. cDNA is made from the RNA using a High Capacity cDNA Reverse Transcription Kit (Applied Biosystems, Life Technologies Corp.) according to the manufacturer's instructions. Finally, qPCR is used to quantify an appropriate dilution of the target cDNA with a Roche Lightcycler 480 PCR instrument and Roche PCR Probes master mix. Relative knockdown of target genes was calculated using the ΔΔCt method using GAPDH as the internal standard. The % mRNA knockdown for target genes cofilinl, LDLR, GNE, SLC35A1,GALE, FUT8, GMDS, and XYLAT are shown elsewhere herein.

The most potent siRNAs are tested further in a range of concentrations. The method for this testing was the same as above except that a range of siRNA concentrations were tested simultaneously.

siRNA Screening in PK15 Cells:

siRNAs against PCV 1 targets of interest are designed and synthesized. Sets of siRNAs to be screened are added to cell media at 10 nM for 1 day for effect. In a 96-well plate, 29.5 μL of Minimum Essential Medium, Eagle's, with Earle's Balanced Salt (EMEM) media (ATCC) are added to test wells and 47 μL to control wells. To this, 17.5 μL of siRNA at 100 nM in CD DG44 media is added to the test wells. To all wells, 4 μL of LIPOFECTAMINE® RNAiMAx reagent (Invitrogen) diluted 1:10 in EMEM media is added. The mixture is allowed to incubate at room temperature for 15 min and then 125 μL of EMEM media containing approximately 20,000 PK15 cells is added to all wells. The plates were then placed in a 37° C. CO₂ incubator for 1 day.

After incubation, cells are visually inspected for toxicity and then RNA is extracted using a MagMax 96-well RNA extraction kit (Ambion) following the manufacturer's instructions. cDNA was made from the RNA using a High Capacity cDNA Reverse Transcription Kit (Applied Biosystems) according to the manufacturer's instructions. Finally, qPCR is used to quantify an appropriate dilution of the target cDNA with a Roche Lightcycler 480 PCR instrument and Roche PCR Probes master mix. Relative knockdown of target genes is calculated using the ΔΔCt method using GAPDH as the internal standard.

TABLE 31
Screen of cofilin1 siRNAs in DG44 cells

| | | | % mRNA | % mRNA |
|---|---|---|---|---|
| SEQ | | | knockdown | knockdown |
| ID NO. | Name | antisense sequence | (1 nM) | (100 pM) |
| 1914036 | AD-30721 | UUCAUUUACACACAGAACUdTdT | 95.2 | 89.3 |
| 1914037 | AD-30722 | GAAUCAGAGGAUCAGAAGCdTdT | 96.4 | 74.2 |
| 1914038 | AD-30723 | AAGAGCACUGCCUUCUUGCdTdT | 76.7 | 42.6 |
| 1914039 | AD-30724 | UGUAAUUCGUGCUUGAUUCdTdT | 96.8 | 82.2 |
| 1914040 | AD-30725 | UUAGAAGUUGGCAGCAUGGdTdT | 87.5 | 66.3 |
| 1914041 | AD-30726 | UCCGCUUCAACCCAAGAGGdTdT | 95.7 | 71.1 |
| 1914042 | AD-30727 | AAGGGGUGCACUCUCAGGGdTdT | 92.6 | 60.5 |
| 1914043 | AD-30728 | UGUGGUUAGAAGUUGGCAGdTdT | 88.8 | 70.0 |
| 1914044 | AD-30729 | UUCCGUUUAAGUAGGAGCCdTdT | 17.4 | 16.5 |
| 1914045 | AD-30730 | GACACCAUCAGAGACAGCCdTdT | 90.8 | 48.6 |
| 1914046 | AD-30731 | UAUUGUGGUUAGAAGUUGGdTdT | 92.4 | 75.4 |
| 1914047 | AD-30732 | ACUUGGUCCGCUUCAACCCdTdT | 91.8 | 75.2 |
| 1914048 | AD-30733 | AAAGGCUUGCCCUCCAGAGdTdT | 40.2 | 9.0 |
| 1914050 | AD-30734 | CUUCCGUUUAAGUAGGAGCdTdT | −18.8 | −5.6 |
| 1914051 | AD-30735 | AGCACAGUCACUAUUGUGGdTdT | 93.8 | 71.2 |
| 1914052 | AD-30736 | UUGAACACUUUGAUGACACdTdT | 88.2 | 73.9 |
| 1914053 | AD-30737 | UUUGCUUGUAAUUCGUGCUdTdT | 91.6 | 76.1 |
| 1914054 | AD-30738 | UUGACAAAAGUGGUGUAGGdTdT | 83.9 | 49.9 |
| 1914055 | AD-30739 | AUAGCGGCAGUCCUUGUCGdTdT | 77.9 | 45.1 |
| 1914056 | AD-30740 | UAAAUUGAAGGUCCCUUACdTdT | 94.3 | 84.5 |
| 1914057 | AD-30741 | UAUCAUUGAACACUUUGAUdTdT | 83.9 | 51.6 |
| 1914058 | AD-30742 | UCGUAGAGAGCAUAGCGGCdTdT | 77.3 | 14.8 |
| 1914060 | AD-30743 | UGACAAAAGUGGUGUAGGGdTdT | 85.6 | 58.6 |
| 1914061 | AD-30744 | UGCAACACCCAUGAGCAGGdTdT | 81.4 | 39.5 |

TABLE 32
Dose-response of cofilin1 siRNAs in DG44 cells

| | % mRNA knockdown | | | | | |
|---|---|---|---|---|---|---|
| | 10 nM | 1 nM | 100 pM | 10 picom | 1 picom | 0.1 picom |
| AD-30721 | 99.2 | 98.8 | 97.4 | 92.9 | 63.4 | 7.7 |
| AD-30724 | 99.3 | 99.0 | 99.0 | 95.5 | 62.1 | 9.0 |
| AD-30731 | 97.1 | 98.7 | 97.5 | 92.7 | 58.7 | −3.1 |
| AD-30737 | 99.3 | 99.4 | 98.8 | 87.7 | 36.3 | −1.7 |
| AD-30740 | 97.9 | 97.6 | 96.2 | 94.6 | 76.8 | 33.1 |

TABLE 33
Screen of LDLR siRNAs in DG44 cells at 1 nM

| SEQ ID | | | % mRNA knockdown |
|---|---|---|---|
| NO. | Name | antisense sequence | (1 nM) |
| 1522123 | AD-29793 | AAAUAUAUAAAACAGAGCCdTdT | 57.7 |
| 1522125 | AD-29794 | AACAGUGCAAUCUAAGAGCdTdT | 64.9 |
| 1522126 | AD-29795 | AUAUCUACACCAUUCAGGCdTdT | 33.5 |
| 1522127 | AD-29796 | GAAUCAACCCAAUAGAGGCdTdT | 40.8 |
| 1522128 | AD-29797 | CUUGCUUUGAGCAACACAGdTdT | 26.9 |
| 1522129 | AD-29798 | ACCAAAUUAACAUCUGAGCdTdT | 23.0 |
| 1522130 | AD-29799 | UCAUUUAUGACAUCCGUCCdTdT | 71.7 |
| 1522131 | AD-29800 | UCAUCUUCCAAAAUGGUCUdTdT | 71.3 |
| 1522132 | AD-29801 | ACUGGAAGCCGACUGAUCCdTdT | 57.7 |
| 1522133 | AD-29802 | AGUGUGAUGCCAUUUGGCCdTdT | 64.9 |
| 1522134 | AD-29803 | AAUUGUGGCAAUUCUAAGUdTdT | 33.5 |
| 1522135 | AD-29804 | AAAAUGGUCUUCCGAUUGCdTdT | 40.8 |
| 1522136 | AD-29805 | UCUUAUCUUGCUUUGAGCAdTdT | 78.1 |
| 1522137 | AD-29806 | AGGGUGAUGGACAAGACCCdTdT | 6.9 |
| 1522138 | AD-29807 | UGUUCAUGCCACAUCAUCCdTdT | 67.5 |
| 1522139 | AD-29808 | UAAAUUGUGGCAAUUCUAAdTdT | 3.2 |
| 1522140 | AD-29809 | AAAUUAACAUCUGAGCCUGdTdT | 16.3 |
| 1522141 | AD-29810 | AAGGAGGAUGACCAGUGCGdTdT | 23.0 |
| 1522142 | AD-29811 | UCUAAGAGCACAGAUGCAGdTdT | 81.3 |
| 1522143 | AD-29812 | AAGAGAAGGUUCUCACAGCdTdT | 48.0 |
| 1522145 | AD-29813 | UUGGAAUCAACCCAAUAGAdTdT | 36.6 |
| 1522146 | AD-29814 | GUGAUCUCAGGCCUGAUGGdTdT | 39.8 |
| 1522148 | AD-29815 | AAAGUGCGAAGAGAGAAUCdTdT | 66.7 |

TABLE 34
Dose response of LDLR siRNAs in DG44 cells

| | % mRNA knockdown | | | | | |
|---|---|---|---|---|---|---|
| | 10 nM | 1 nM | 100 pM | 10 picom | 1 picom | 0.1 picom |
| AD-29799 | 91.9 | 91.3 | 83.0 | 43.5 | 17.6 | −8.7 |
| AD-29800 | 87.9 | 84.4 | 66.5 | 9.8 | −10.3 | −18.6 |
| AD-29805 | 89.1 | 87.4 | 83.6 | 55.2 | 15.6 | −11.4 |
| AD-29807 | 95.8 | 93.2 | 83.1 | 45.3 | 4.4 | −15.3 |
| AD-29811 | 93.8 | 89.1 | 85.7 | 69.1 | 33.3 | 3.4 |

TABLE 35
Screen of GNE siRNAs in DG44 cells at 1 nM

| SEQ | | | % mRNA knockdown |
|---|---|---|---|
| ID NO. | Name | antisense sequence | (1 nM) |
| 420095 | AD-29121 | UACUGACUCUACCAUGGCUdTdT | 89.2 |
| 419972 | AD-29122 | UCCAUGAACAAUCAUGAUGdTdT | 94.7 |
| 420052 | AD-29123 | AGUUUGUCAUAGGAAGGGCdTdT | 85.7 |
| 419975 | AD-29124 | AGCAGUUUGUCAUAGGAAGdTdT | 97.5 |
| 420033 | AD-29125 | AUCCGAAUGAUGCUCAUAUdTdT | 80.0 |
| 420029 | AD-29126 | AGUGCAACAAUGUAAUCUUdTdT | 38.4 |
| 420005 | AD-29127 | AAUGAGAUAAGUGCAUCCAdTdT | 66.8 |
| 420224 | AD-29128 | ACAUGCUUGACUGCACGAAdTdT | 28.3 |
| 420003 | AD-29129 | AAAUGGGACAUGCUUGACUdTdT | 95.8 |
| 420090 | AD-29130 | AUAAACUGGUCAAAUGGGAdTdT | 88.3 |
| 419965 | AD-29131 | UAUAAACUGGUCAAAUGGGdTdT | 70.1 |
| 420219 | AD-29132 | AUCAUACAACCAGCAUGGGdTdT | 51.7 |
| 420140 | AD-29134 | UUAUCUUGGGUGUCAGCAUdTdT | 90.7 |
| 419984 | AD-29135 | UUUAUCUUGGGUGUCAGCAdTdT | 91.9 |
| 420214 | AD-29136 | AGGUGGAGCGCUUGUAAUAdTdT | −55.8 |
| 419970 | AD-29137 | UUUACCGAACUGAAGGUGGdTdT | 81.6 |
| 420077 | AD-29138 | UACUGUUUACCGAACUGAAdTdT | 29.3 |
| 420096 | AD-29139 | UCUUAACUAUUUCACCCUUdTdT | 45.3 |
| 420025 | AD-29140 | UUCUUAACUAUUUCACCCUdTdT | 79.1 |
| 420098 | AD-29141 | UCUGCAGGAUUAAACUAAUdTdT | 26.8 |
| 420186 | AD-29142 | AAAUGCCUACUCCCAGAAUdTdT | 44.1 |
| 420121 | AD-29143 | UUGGCCAAACUUCCUUUCUdTdT | 83.5 |
| 419964 | AD-29144 | UGUAAUGAGUGUCACAAAGdTdT | 87.5 |
| 420243 | AD-29145 | UGCCUGUAAUGAGUGUCACdTdT | 79.1 |
| 420011 | AD-29146 | AUAUUCUGGGCCUUCACGUdTdT | 83.8 |
| 419974 | AD-29147 | UGUUCGUAGGAUAUUCUGGdTdT | 79.6 |
| 420084 | AD-29148 | AGCUGUUCGUAGGAUAUUCdTdT | 77.3 |
| 420143 | AD-29149 | ACGUCCUUGACAAUGUGGAdTdT | 87.3 |
| 420275 | AD-29150 | AGGGUCAACCAAGUCUGAAdTdT | 10.5 |
| 420017 | AD-29151 | UGAAGAAGUACUGAUCUAAdTdT | 86.1 |
| 420297 | AD-29133 | UCUUCCUAUCUGGCGUGUUdTdT | 74.5 |
| 420064 | AD-29152 | AGAUCCACCUUUAAUCUAGdTdT | −167.6 |

TABLE 36
Dose-response of GNE siRNAs in DG44 cells

| | % mRNA knockdown | | | | | |
|---|---|---|---|---|---|---|
| | 10 nM | 1 nM | 100 pM | 10 picom | 1 picom | 0.1 pM |
| AD-29122 | 95.5 | 93.6 | 75.9 | 28.8 | 47.7 | 17.9 |
| AD-29124 | 94.4 | 89.6 | 63.3 | 7.3 | −3.2 | −13.3 |
| AD-29129 | 97.7 | 90.7 | 87.0 | 36.9 | −18.5 | −20.2 |
| AD-29134 | 94.6 | 89.3 | 66.1 | −5.0 | −43.4 | −3.5 |
| AD-29135 | 89.9 | 90.6 | 80.5 | 12.3 | −4.6 | −38.0 |

TABLE 37
Screen of SLC35A1 siRNAs at 1 nM on DG44 cells for 3 days

| SEQ | | | % mRNA knockdown |
|---|---|---|---|
| ID NO. | Name | antisense sequence | (1 nM) |
| 1368055 | AD-29063 | AAGCUACGGUAUAAGCUGCdTdT | 92.0 |
| 1367961 | AD-29064 | AAAGCUACGGUAUAAGCUGdTdT | 83.6 |
| 1368040 | AD-29065 | UGUGUAUCUUAAAGCUACGdTdT | 79.3 |
| 1367983 | AD-29066 | UUGUGUAUCUUAAAGCUACdTdT | 90.5 |
| 1367981 | AD-29067 | ACUUUAUAACUUCUGUGACdTdT | 94.4 |
| 1368010 | AD-29068 | AAUCUACCCAAACUUCCAGdTdT | 99.5 |
| 1367999 | AD-29069 | UUAAAUCUACCCAAACUUCdTdT | 99.5 |
| 1368000 | AD-29070 | AGAUGCCUUAAAUCUACCCdTdT | 99.3 |
| 1368085 | AD-29071 | AAGAUGCCUUAAAUCUACCdTdT | 97.9 |
| 1367995 | AD-29072 | ACUAAGAGCUAGGAAAGCCdTdT | 99.5 |
| 1368045 | AD-29073 | AUACUAAGAGCUAGGAAAGdTdT | 99.4 |
| 1368017 | AD-29074 | UAGGUAACCUGGUAUACUGdTdT | 99.4 |
| 1368068 | AD-29075 | ACCCAUGUAAUUUGCUGAGdTdT | 75.3 |
| 1367960 | AD-29076 | AUAGCUAUUGCACCAAAGCdTdT | 99.6 |
| 1367978 | AD-29077 | AAUACAGCAAUAGCUAUUGdTdT | 98.4 |
| 1368053 | AD-29078 | AUCCAGAGCACAAUACAGCdTdT | 99.3 |
| 1367953 | AD-29079 | AAUAAACUCCUGCAAAUCCdTdT | 97.9 |
| 1367957 | AD-29080 | AAGUGAAUGUUUCUCACCCdTdT | 96.2 |
| 1368159 | AD-29081 | UAGUCGUUAUAGGAGUAUCdTdT | 99.2 |
| 1368033 | AD-29082 | AGUUUAGUCGUUAUAGGAGdTdT | 99.5 |
| 1367956 | AD-29083 | AUUAUUGACAGUUUAGUCGdTdT | 99.1 |
| 1368012 | AD-29084 | UAUUAUUGACAGUUUAGUCdTdT | 99.7 |
| 1367967 | AD-29085 | UGUUUAAGCUACCAUCUGGdTdT | 98.1 |
| 1367977 | AD-29086 | UAUUGUUUAAGCUACCAUCdTdT | 98.7 |
| 1368065 | AD-29087 | UGAUAUUGUUUAAGCUACCdTdT | 98.0 |
| 1367973 | AD-29088 | UUGAUAUUGUUUAAGCUACdTdT | 99.3 |
| 1368038 | AD-29089 | UUGAAUAUUGUAGUUUCACdTdT | 98.3 |
| 1368089 | AD-29090 | ACUUGAACCUUCAGAUACCdTdT | 98.0 |
| 1368077 | AD-29091 | UACCUGAACGAGAGAACAGdTdT | 98.7 |
| 1368067 | AD-29092 | UCUCUUAUUCAUUCUUCACdTdT | 98.6 |
| 1368039 | AD-29093 | UUACCCAGACAGAAGUCAGdTdT | 99.1 |
| 1367952 | AD-29094 | AAGUAUAUCAGCUAACAGCdTdT | 99.7 |
| 1367965 | AD-29095 | AGUUCACAAAUUGAGAGCCdTdT | 98.6 |
| 1367963 | AD-29096 | AAGUUCACAAAUUGAGAGCdTdT | 97.2 |

TABLE 38
Dose-response of SLC35A1 siRNAs in DG44 cells

| | % mRNA knockdown | | | | | |
|---|---|---|---|---|---|---|
| | 10 nM | 1 nM | 100 pM | 10 picom | 1 picom | 0.1 pM |
| AD-29068 | 99.7 | 99.3 | 95.3 | 51.8 | 34.9 | 39.5 |
| AD-29069 | 94.8 | 94.1 | 88.8 | 56.9 | 59.8 | 60.1 |
| AD-29076 | 99.9 | 99.4 | 96.1 | 76.7 | 55.8 | 30.2 |
| AD-29084 | 99.9 | 99.7 | 99.0 | 95.4 | 54.9 | 1.6 |
| AD-29094 | 99.8 | 99.6 | 99.5 | 97.4 | 54.1 | −9.1 |

TABLE 39
Screen of SLC35A2 siRNAs at 1 nM on DG44 cells for 3 days

| SEQ | | | % mRNA knockdown |
|---|---|---|---|
| ID NO. | Name | antisense sequence | (1 nM) |
| 464723 | AD-29097 | UAUCGGAUGCUAAGGAUGAdTdT | 86.9 |
| 464762 | AD-29098 | AUAUCGGAUGCUAAGGAUGdTdT | 89.9 |
| 464953 | AD-29099 | UACGAGCAUAUCGGAUGCUdTdT | 80.2 |
| 464679 | AD-29100 | AAAUAAUGGGUCCAGGUGAdTdT | 90.8 |
| 464814 | AD-29101 | UUAUGGCUUUGACUGCACUdTdT | 92.9 |
| 464729 | AD-29102 | UAUCCCUCUAGAAGUGUGGdTdT | 92.2 |
| 464852 | AD-29103 | AUAUCCCUCUAGAAGUGUGdTdT | 87.8 |
| 464750 | AD-29104 | UUCCCAAAGAGGUUAGCCUdTdT | 81.4 |
| 464833 | AD-29105 | AUUAGUCGUUACUGAAGAAdTdT | 75.6 |
| 464844 | AD-29106 | UUACAACAGGCCGAUCUUCdTdT | 83.0 |
| 464676 | AD-29107 | AAGUAAAUGGUGCUUAUUGdTdT | 88.3 |
| 464859 | AD-29108 | AUCACAAAUGCCCGACAUAdTdT | 85.4 |
| 464748 | AD-29109 | UAUCACAAAUGCCCGACAUdTdT | 92.4 |
| 464820 | AD-29110 | AUAUCACAAAUGCCCGACAdTdT | 90.4 |
| 464675 | AD-29111 | AACCUGAUAUCACAAAUGCdTdT | 92.2 |
| 464701 | AD-29112 | AAUUCUGACACCGCCAUGAdTdT | 49.8 |
| 464847 | AD-29113 | AUCAAUUCUGACACCGCCAdTdT | 83.4 |
| 464702 | AD-29114 | UAAGGAGUUAGUAAGCUUUdTdT | 86.4 |
| 464778 | AD-29115 | UACAGUUAAGGAGUUAGUAdTdT | 79.5 |
| 464881 | AD-29116 | AUACAGUUAAGGAGUUAGUdTdT | 90.1 |
| 464961 | AD-29117 | AUCCUGACAUAUGUUCAUUdTdT | 66.0 |
| 464804 | AD-29118 | UAUCCUGACAUAUGUUCAUdTdT | 83.7 |
| 464726 | AD-29119 | UUGGCAUUGGGUAUCCUGAdTdT | 83.2 |
| 464799 | AD-29120 | UUAUUUGGCAUUGGGUAUCdTdT | 89.2 |

TABLE 40
Dose-response of SLC35A2 siRNAs in DG44 cells

| | % mRNA knockdown | | | | | |
|---|---|---|---|---|---|---|
| | 10 nM | 1 nM | 100 pM | 10 picom | 1 picom | 0.1 pM |
| AD-29101 | 66.4 | 60.3 | 16.4 | 24.1 | −0.1 | 19.5 |
| AD-29102 | 92.6 | 87.4 | 81.8 | −4.3 | 66.5 | 53.3 |
| AD-29109 | 85.7 | 84.2 | 79.5 | 37.5 | −2.2 | −31.6 |
| AD-29110 | 74.4 | 84.4 | 77.3 | −20.3 | −63.2 | −51.2 |
| AD-29111 | 97.3 | 86.1 | 80.9 | 27.2 | −15.8 | −2.9 |

TABLE 41
Screen of GALE siRNAs at 1 nM with 3 days incubation on adherant
DG44 cells

| SEQ | | | % mRNA knockdown |
|---|---|---|---|
| ID NO. | Name | antisense sequence | (1 nM) |
| 1888656 | AD-28691 | UCUAUAAUAAUCCAGAGGCdTdT | 92.1 |
| 1888657 | AD-28692 | AACACGAGAUUCUUCACCCdTdT | 88.8 |
| 1888660 | AD-28693 | AAAGCUGUGCUUCUUAAAGdTdT | 70.3 |
| 1888659 | AD-28694 | AUUGAAGUAGCGUAGCAGCdTdT | 87.4 |
| 1888663 | AD-28695 | UGUAUCAUAGUCACCACCAdTdT | 92.6 |
| 1888662 | AD-28696 | UCCACGAAUGGCAUUAUGGdTdT | 86.4 |
| 1888665 | AD-28697 | ACCACAUGAAUGUAAUCCCdTdT | 90.1 |
| 1888674 | AD-28698 | AACUCUAUAAUAAUCCAGAdTdT | 66.3 |
| 1888673 | AD-28699 | ACUUGGACUUUCCAUAGGGdTdT | 78.1 |
| 1888689 | AD-28700 | UAUGGGAUCUUCUUCCCUGdTdT | 73.7 |
| 1888690 | AD-28701 | UAUAAUAAUCCAGAGGCUUdTdT | 89.3 |
| 1888698 | AD-28702 | UCCUCUGUAUCAUAGUCACdTdT | 90.5 |
| 1888695 | AD-28703 | AUGAAUGUAAUCCCUUACAdTdT | 86.5 |
| 1888703 | AD-28704 | UGUAAUCCCUUACACCUGUdTdT | 89.8 |
| 1888702 | AD-28705 | AGAACUUGGACUUUCCAUAdTdT | 93.3 |
| 1888706 | AD-28706 | AACCACAUUGCUCCUUCAGdTdT | 65.7 |
| 1888707 | AD-28707 | AUAAUAAUCCAGAGGCUUCdTdT | 73.0 |
| 1888705 | AD-28708 | ACAGCCUUAAAGCUGUGCUdTdT | 73.7 |
| 1888719 | AD-28709 | UAAUCCCUUACACCUGUGCdTdT | 91.3 |
| 1888708 | AD-28710 | UUCGUAAGGAGGUCUUUAGdTdT | 95.2 |
| 1888701 | AD-28711 | UCGUAAGGAGGUCUUUAGGdTdT | 94.0 |
| 1888710 | AD-28712 | UCUUAAAGAGGUGCUGUAGdTdT | 91.3 |
| 1888716 | AD-28713 | UAAGGAGGUCUUUAGGCCUdTdT | 82.4 |
| 1888738 | AD-28714 | UCCCUGUUAGGUUAACUCUdTdT | 92.2 |
| 1888735 | AD-28715 | UUUUGGUCCUUCGUAAGGAdTdT | 89.7 |
| 1888723 | AD-28716 | UUGAAGUAGCGUAGCAGCAdTdT | 66.8 |
| 1888769 | AD-28717 | UAAAGCUGUGCUUCUUAAAdTdT | 25.5 |
| 1888781 | AD-28718 | UGAACACGAGAUUCUUCACdTdT | 93.6 |
| 1888774 | AD-28719 | AAGUGGAUGACAGCCUUAAdTdT | 60.9 |

TABLE 42
Dose-response of GALE siRNAs in DG44 cells

| | % mRNA knockdown | | | | |
|---|---|---|---|---|---|
| | 10 nM | 1 nM | 100 pM | 10 picoM | 1 picoM |
| AD-28695 | 99.1 | 97.8 | 92.1 | 75.5 | 32.8 |
| AD-28705 | 96.4 | 94.9 | 94.2 | 76.7 | 45.4 |
| AD-28710 | 97.1 | 97.8 | 94.9 | 88.3 | 42.3 |
| AD-28711 | 98.7 | 97.8 | 95.2 | 75.6 | 24.9 |
| AD-28718 | 98.6 | 98.3 | 93.6 | 61.4 | 3.6 |

TABLE 45
Screen of GMDS siRNAs at 10 nM with 1 day incubation on adherent
DG44 cells

| SEQ | | | % mRNA knockdown |
|---|---|---|---|
| ID NO. | Name | antisense sequence | (10 nM) |
| 1688259 | AD-25328 | UUCGACCUGUAUUAAAUGAdTdT | 93.1 |
| 1688271 | AD-25329 | AAUUCGACCUGUAUUAAAUdTdT | 93.9 |
| 1688246 | AD-25331 | UAUAAAUGUUCAAUUCGACdTdT | 91.3 |
| 3157131 | AD-25332 | UUCAUGUUUCCUUCAAUAUdTdT | 78.9 |
| 1688245 | AD-25333 | AGUGCAACUUCAUGUUUCCdTdT | 89.8 |
| 3157132 | AD-25334 | UCCAAGGUAAAUCUUAGCUdTdT | 90.6 |
| 3157133 | AD-25338 | AUAGUCCUUGGCAUGGCCCdTdT | 87.9 |
| 3157134 | AD-25340 | UUCCUUCCCACACAAUGGUdTdT | 89.9 |
| 3157135 | AD-25342 | UUGCCGGUCUCUUUACAUCdTdT | 85.1 |
| 1688220 | AD-25344 | AAGUAAAAUGAGUAUGUGAdTdT | 92.7 |
| 1688283 | AD-25345 | UAGUGACAUAAUUUCAAGUdTdT | 92.7 |
| 3157136 | AD-25346 | UUGUCUAGUGACAUAAUUUdTdT | 87.1 |
| 3157137 | AD-25347 | AAAAACAAUCUCAAGACUCdTdT | 91.2 |
| 1688483 | AD-25330 | AUGUUCAAUUCGACCUGUAdTdT | 81.4 |
| 3157138 | AD-25335 | UGUCCAAGGUAAAUCUUAGdTdT | 77.3 |
| 3157139 | AD-25336 | UUGUCCAAGGUAAAUCUUAdTdT | 83.3 |
| 3157140 | AD-25337 | UUGGCAUGGCCCCAGUCUCdTdT | 63.0 |
| 1688295 | AD-25339 | UUCCCCAGUAGCUAUGACAdTdT | 80.8 |
| 1688307 | AD-25341 | UCUCUUUACAUCUGCCCACdTdT | 90.8 |
| 1688317 | AD-25343 | AAAGGCAACGCGGGGCUUCdTdT | 80.9 |

TABLE 46
Dose-response of GMDS siRNAs in DG44 cells

| | % mRNA knockdown | | | | |
|---|---|---|---|---|---|
| | 100 nM | 10 nM | 1 nM | 100 pM | 10 picom |
| AD-25328 | 94.9 | 89.4 | 75.9 | 49.5 | −3.3 |
| AD-25329 | 92.1 | 89.1 | 80.1 | 49.9 | 12.6 |
| AD-25331 | 94.2 | 88.9 | 87.6 | 73.8 | 27.5 |
| AD-25344 | 96.4 | 91.2 | 85.8 | 63.8 | 21.4 |
| AD-25345 | 92.9 | 86.3 | 78.0 | 45.1 | 26.7 |

TABLE 47
Screen of XYLT2 siRNAs at 100 pM with 4 days incubation on adherant
DG44 cells

| SEQ ID | | | % mRNA knockdown |
|---|---|---|---|
| NO. | Name | antisense sequence | (10 nM) |
| 1554777 | AD-28123 | AUUAGCAGUAAGUAGUGAGdTdT | 50.5 |
| 1554779 | AD-28124 | AAGUAGUGAGCACUACACCdTdT | −18.5 |
| 3157141 | AD-28125 | UUUCCUGAGAGGUAGUUUGdTdT | 84.1 |
| 1554785 | AD-28126 | UCUUAGGUCUGCUUGGAGCdTdT | 65.3 |
| 3157142 | AD-28127 | UCAGUGUCCUCAUCUACCGdTdT | 52.9 |
| 3157143 | AD-28128 | AGGUUGGAUCAAUAGGGCCdTdT | −77.2 |
| 3157144 | AD-28129 | AGAACUGAAGCAAUCGAACdTdT | 76.1 |
| 3157145 | AD-28130 | UGCGGUUGAAGGUCAAUGGdTdT | 43.0 |
| 1554792 | AD-28131 | AGACAAAACCUCUCCAGAGdTdT | 34.5 |
| 1554793 | AD-28132 | ACUUCUUAGGUCUGCUUGGdTdT | 62.6 |
| 1554795 | AD-28133 | UGUCAUAUGAUGUGGCCACdTdT | 1.7 |
| 1554806 | AD-28134 | ACCGUGAUGUCAUAUGAUGdTdT | 67.5 |
| 1554808 | AD-28135 | AAAGAAGGUGGGUCUGGAGdTdT | 62.9 |
| 1554809 | AD-28136 | UCUACCGUGAUGUCAUAUGdTdT | 72.2 |
| 1554815 | AD-28137 | UAGGUUGGAUCAAUAGGGCdTdT | −109.9 |
| 1554821 | AD-28138 | ACGAUGUGUUUGUACUGGCdTdT | 70.0 |
| 3157146 | AD-28139 | AGCAGUAAGUAGUGAGCACdTdT | 72.4 |
| 3157147 | AD-28140 | UACGGUUCCAGUUGGUGACdTdT | 81.1 |
| 1554825 | AD-28141 | ACAAGGAAGCGAAUCUCGCdTdT | 76.0 |
| 3157148 | AD-28142 | AGCACGAACCAGUCAGAACdTdT | 61.6 |
| 1554838 | AD-28143 | AUGUAUUCAUUGUGGGGUGdTdT | 39.3 |
| 1554860 | AD-28144 | ACAGCCCACUUCUUAGGUCdTdT | 54.0 |
| 1554886 | AD-28145 | UGACACGCAAGUUGUUGUCdTdT | 2.4 |

TABLE 48
Dose-response of XYLT2 siRNAs in DG44 cells

| | % mRNA knockdown | | | | |
|---|---|---|---|---|---|
| | 10 nM | 1 nM | 100 pM | 10 picom | 1 picom |
| AD-28125 | 89.0 | 91.3 | 77.2 | 42.0 | 29.3 |
| AD-28129 | 93.3 | 88.1 | 81.6 | 50.9 | 24.5 |
| AD-28139 | 92.0 | 92.6 | 68.5 | 34.7 | 15.9 |
| AD-28140 | 96.0 | 96.4 | 75.7 | 53.7 | 13.8 |
| AD-28141 | 94.9 | 92.5 | 81.6 | 60.8 | 34.7 |

TABLE 49
siRNAs against PCV1 Rep screened at 10 nM overnight in PCV1 infected
PK15 cells

| SEQ | | | % mRNA knockdown |
|---|---|---|---|
| ID NO: | Name | antisense sequence | (10 nM) |
| 3290845 | AD-36165.2 | AAcACCcACCUCUuAUGGGdTsdT | −3.5 |
| 3290846 | AD-36171.2 | uAAGGGUGAAcACCcACCUdTsdT | 11.4 |
| 3290847 | AD-36177.2 | UuAAGGGUGAAcACCcACCdTsdT | 17.6 |
| 3290848 | AD-36183.2 | AUuAAGGGUGAAcACCcACdTsdT | 23.4 |
| 3290849 | AD-36189.2 | uAUuAAGGGUGAAcACCcAdTsdT | −35.7 |
| 3290850 | AD-36195.2 | UuAUuAAGGGUGAAcACCCdTsdT | 3.4 |
| 3290851 | AD-36201.2 | AAGCUCCCGuAUUUUGUUUdTsdT | −5.7 |
| 3290852 | AD-36207.2 | AAGGGAGAUUGGAAGCUCCdTsdT | −17.3 |
| 3290853 | AD-36166.2 | UUCCUCUCCGcAAAcAAAAdTsdT | 29.3 |
| 3290854 | AD-36172.2 | AAACCUUCCUCUCCGcAAAdTsdT | 63.0 |
| 3290855 | AD-36178.2 | UUCcAAACCUUCCUCUCCGdTsdT | 49.8 |
| 3290856 | AD-36184.2 | uACCCUCUUCcAAACCUUCdTsdT | −36.1 |
| 3290857 | AD-36190.2 | UUCuACCCUCUUCcAAACCdTsdT | −11.3 |
| 3290858 | AD-36196.2 | AAUUCGcAAACCCCUGGAGdTsdT | 22.6 |
| 3290859 | AD-36202.2 | AAAUUCGcAAACCCCUGGAdTsdT | 28.3 |
| 3290860 | AD-36208.2 | uAGcAAAAUUCGcAAACCCdTsdT | 48.2 |
| 3290861 | AD-36167.2 | UUCUuAGcAAAAUUCGcAAdTsdT | 16.5 |
| 3290862 | AD-36173.2 | AAGUCUGCUUCUuAGcAAAdTsdT | 63.5 |
| 3290863 | AD-36179.2 | AAAGUCUGCUUCUuAGcAAdTsdT | 42.0 |
| 3290864 | AD-36185.2 | AAAAGUCUGCUUCUuAGcAdTsdT | 57.8 |
| 3290865 | AD-36191.2 | uAAAAGUCUGCUUCUuAGCdTsdT | 61.2 |
| 3290866 | AD-36197.2 | UuAAAAGUCUGCUUCUuAGdTsdT | 55.4 |
| 3290867 | AD-36203.2 | UUcACCUUGUuAAAAGUCUdTsdT | 64.8 |
| 3290868 | AD-36209.2 | uACcACUUcACCUUGUuAAdTsdT | 66.2 |
| 3290869 | AD-36168.2 | AuACcACUUcACCUUGUuAdTsdT | 21.8 |
| 3290870 | AD-36174.2 | AAuACcACUUcACCUUGUUdTsdT | 30.3 |
| 3290871 | AD-36180.2 | AAAuACcACUUcACCUUGUdTsdT | 48.9 |
| 3290872 | AD-36186.2 | UUCGCUUUCUCGAUGUGGCdTsdT | 51.0 |
| 3290873 | AD-36192.2 | UUCCUUUCGCUUUCUCGAUdTsdT | 53.3 |
| 3290874 | AD-36198.2 | UuAUUCUGCUGGUCGGUUCdTsdT | 17.1 |
| 3290875 | AD-36204.2 | UUCUUuAUUCUGCUGGUCGdTsdT | 13.2 |
| 3290876 | AD-36210.2 | uACUGcAGuAUUCUUuAUUdTsdT | 61.8 |
| 3290877 | AD-36169.2 | UuACUGcAGuAUUCUUuAUdTsdT | 35.4 |
| 3290878 | AD-36175.2 | UUuACUGcAGuAUUCUUuAdTsdT | 0.3 |
| 3290879 | AD-36181.2 | AUGUGGCCUUCUUuACUGCdTsdT | −20.2 |
| 3290880 | AD-36187.2 | uAUGUGGCCUUCUUuACUGdTsdT | 18.2 |
| 3290881 | AD-36193.2 | AAGuAUGUGGCCUUCUUuAdTsdT | −218.2 |
| 3290882 | AD-36199.2 | uAAGuAUGUGGCCUUCUUUdTsdT | 47.1 |
| 3290883 | AD-36205.2 | AuAAGuAUGUGGCCUUCUUdTsdT | 41.0 |
| 3290884 | AD-36211.2 | uACUcAcAGcAGuAGAcAGdTsdT | −30.6 |
| 3290885 | AD-36170.2 | AAAGGGuACUcAcAGcAGUdTsdT | 23.7 |
| 3290886 | AD-36176.2 | AAAAGGGuACUcAcAGcAGdTsdT | 27.6 |
| 3290887 | AD-36182.2 | AACUGCUCGGCuAcAGUcAdTsdT | 31.7 |
| 3290888 | AD-36188.2 | uACGUuAcAGGGAACUGCUdTsdT | 40.3 |
| 3290889 | AD-36194.2 | UUCUcAcAuACGUuAcAGGdTsdT | 53.3 |
| 3290890 | AD-36200.2 | AAUUUCUcAcAuACGUuACdTsdT | 58.2 |
| 3290891 | AD-36206.2 | AAAUUUCUcAcAuACGUuAdTsdT | 63.5 |
| 3290892 | AD-36212.2 | UUCCCGCUcACUUUcAAAAdTsdT | 58.5 |
| 3290893 | AD-36213.1 | AUCUUCCCGCUcACUUUcAdTsdT | −100.7 |
| 3290894 | AD-36219.1 | AUcACGCUGCUGcAUCUUCdTsdT | 16.5 |
| 3290895 | AD-36225.1 | uAcAGCUGUCUUCcAAUcAdTsdT | 36.7 |
| 3290896 | AD-36231.1 | AAAAUuACGGGCCcACUGGdTsdT | 20.2 |
| 3290897 | AD-36237.1 | uAGGCUcAGcAAAAUuACGdTsdT | 24.0 |
| 3290898 | AD-36243.1 | UUCcAGuAGGUGUCGCuAGdTsdT | 41.0 |
| 3290899 | AD-36249.1 | UUCuACuAGGCUUCcAGuAdTsdT | 64.0 |
| 3290900 | AD-36255.1 | UUUCuACuAGGCUUCcAGUdTsdT | 63.7 |
| 3290901 | AD-36214.1 | AUUUCuACuAGGCUUCcAGdTsdT | 43.4 |
| 3290902 | AD-36220.1 | AUCCcACcACUuAUUUCuAdTsdT | 11.7 |
| 3290903 | AD-36226.1 | AUCcAUCCcACcACUuAUUdTsdT | 18.5 |
| 3290904 | AD-36232.1 | uAUCcAUCCcACcACUuAUdTsdT | 24.5 |
| 3290905 | AD-36238.1 | AuAUCcAUCCcACcACUuAdTsdT | 40.1 |
| 3290906 | AD-36244.1 | AUGAuAUCcAUCCcACcACdTsdT | 29.3 |
| 3290907 | AD-36250.2 | UUCUCcAUGAuAUCcAUCCdTsdT | 50.7 |
| 3290908 | AD-36256.1 | UUCUUCUCcAUGAuAUCcAdTsdT | 22.1 |
| 3290909 | AD-36215.1 | AACUUCUUCUCcAUGAuAUdTsdT | 27.3 |
| 3290910 | AD-36221.1 | AAcAACUUCUUCUCcAUGAdTsdT | 46.0 |
| 3290911 | AD-36227.2 | AAcAAcAACUUCUUCUCcAdTsdT | 52.4 |
| 3290912 | AD-36233.1 | AAAcAAcAACUUCUUCUCCdTsdT | 55.1 |
| 3290913 | AD-36239.2 | AAAAcAAcAACUUCUUCUCdTsdT | 46.6 |
| 3290914 | AD-36245.1 | AUCcAAAAcAAcAACUUCUdTsdT | 56.9 |
| 3290915 | AD-36251.2 | AUcAUCcAAAAcAAcAACUdTsdT | 25.8 |
| 3290916 | AD-36257.1 | AAUcAUCcAAAAcAAcAACdTsdT | 76.0 |
| 3290917 | AD-36222.1 | AAGGuAACcAGCcAuAAAAdTsdT | 53.0 |
| 3290918 | AD-36228.2 | AUCCcAAGGuAACcAGCcAdTsdT | −5.0 |
| 3290919 | AD-36234.2 | AUcAUCCcAAGGuAACcAGdTsdT | 30.3 |
| 3290920 | AD-36240.1 | AuACCGGUcAcAcAGUCUCdTsdT | 16.8 |
| 3290921 | AD-36246.1 | AUGGAuACCGGUcAcAcAGdTsdT | 39.5 |
| 3290922 | AD-36252.1 | AAUGGAuACCGGUcAcAcAdTsdT | 29.0 |
| 3290923 | AD-36258.1 | uAcAGUcAAUGGAuACCGGdTsdT | 14.4 |
| 3290924 | AD-36217.1 | uAGUCUCuAcAGUcAAUGGdTsdT | −5.7 |
| 3290925 | AD-36223.1 | UUGCUGGuAAUcAAAAuACdTsdT | 33.3 |
| 3290926 | AD-36229.1 | AUUGCUGGuAAUcAAAAuAdTsdT | 38.2 |
| 3290927 | AD-36235.1 | UUGAGGAGuACcAUUCCUGdTsdT | 18.5 |
| 3290928 | AD-36241.1 | uAGAGAGCUUCuAcAGCUGdTsdT | 16.5 |
| 3290929 | AD-36247.2 | AuAGAGAGCUUCuAcAGCUdTsdT | 58.5 |
| 3290930 | AD-36253.1 | AAGuAGuAAUCCUCCGAuAdTsdT | −20.6 |
| 3290931 | AD-36259.1 | AAAGuAGuAAUCCUCCGAUdTsdT | 10.2 |
| 3290932 | AD-36218.1 | UUGcAAAGuAGuAAUCCUCdTsdT | 22.1 |
| 3290933 | AD-36224.2 | AUUGcAAAGuAGuAAUCCUdTsdT | 36.3 |
| 3290934 | AD-36230.2 | UUCcAAAAUUGcAAAGuAGdTsdT | −16.0 |
| 3290935 | AD-36236.1 | UUCUCcAGcAGUCUUCcAAdTsdT | −1.0 |
| 3290936 | AD-36242.1 | AUUGUUCUCcAGcAGUCUUdTsdT | 22.3 |
| 3290937 | AD-36248.1 | uACCUCCGUGGAUUGUUCUdTsdT | 14.5 |
| 3290938 | AD-36254.1 | UUcAAAUCGGCCUUCGGGUdTsdT | 58.1 |
| 3290939 | AD-36260.1 | UUuAuAUGGGAAAAGGGcAdTsdT | 27.4 |

TABLE 50
siRNAs against PCV1 Cap screened at 10 nM overnight in PCV1 infected
PK15 cells

| SEQ | | | % mRNA knockdown |
|---|---|---|---|
| ID NO. | Name | antisense sequence | (10 nM) |
| | AD-35779.1 | AUGUUUCcAAGAUGGCUGCdTsdT | 8.2 |
| | AD-35785.1 | UUCUCCGGAGGAUGUUUCCdTsdT | 61.9 |
| | AD-35791.1 | uAUGGUCUUCUCCGGAGGAdTsdT | −1.8 |
| | AD-35797.1 | AuAUGGUCUUCUCCGGAGGdTsdT | 43.5 |
| | AD-35803.1 | AAuAUGGUCUUCUCCGGAGdTsdT | 52.8 |
| | AD-35809.1 | AAAuAUGGUCUUCUCCGGAdTsdT | 27.0 |
| | AD-35815.1 | uAACGGUUUCUGAAGGCGGdTsdT | 1.6 |
| | AD-35821.1 | AUCUGuAACGGUUUCUGAAdTsdT | −226.3 |
| | AD-35780.1 | AAGAuACCCGUCUUUCGGCdTsdT | 5.6 |
| | AD-35786.1 | UUGAAGAuACCCGUCUUUCdTsdT | 42.3 |
| | AD-35792.1 | AUUGAAGAuACCCGUCUUUdTsdT | −15.4 |
| | AD-35798.1 | AAUUGAAGAuACCCGUCUUdTsdT | 6.3 |
| | AD-35810.1 | UUCUCuAGAAAGGCGGGAAdTsdT | −761.2 |
| | AD-35816.1 | AAUUCUCuAGAAAGGCGGGdTsdT | 52.5 |
| | AD-35822.1 | AAAUUCUCuAGAAAGGCGGdTsdT | 66.4 |
| | AD-35781.1 | uAcAAAUUCUCuAGAAAGGdTsdT | 36.0 |
| | AD-35787.1 | AUGGUGAGuAcAAAUUCUCdTsdT | 43.9 |
| | AD-35793.1 | uAUGGUGAGuAcAAAUUCUdTsdT | −15.4 |
| | AD-35799.1 | UUCcAAGAUGGCUGCGAGUdTsdT | 13.2 |
| | AD-35805.1 | AUUCcAAGAUGGCUGCGAGdTsdT | 15.0 |
| | AD-35811.1 | AAcAUUCcAAGAUGGCUGCdTsdT | −16.2 |
| | AD-35817.1 | uAAcAUUCcAAGAUGGCUGdTsdT | 12.0 |
| | AD-35823.1 | UuAAcAUUCcAAGAUGGCUdTsdT | −135.6 |
| | AD-35782.1 | uAUUGGAAAGGuAGGGGuAdTsdT | 30.4 |
| | AD-35788.1 | AuACGGuAGuAUUGGAAAGdTsdT | 41.9 |
| | AD-35794.1 | AAuACGGuAGuAUUGGAAAdTsdT | 19.5 |
| | AD-35800.1 | uAAuACGGuAGuAUUGGAAdTsdT | 22.8 |
| | AD-35806.1 | UUCuAAuACGGuAGuAUUGdTsdT | −9.9 |
| | AD-35812.1 | UUUCuAAuACGGuAGuAUUdTsdT | 49.4 |
| | AD-35818.1 | uAGCCUUUCuAAuACGGuAdTsdT | 29.5 |
| | AD-35824.1 | UuAGCCUUUCuAAuACGGUdTsdT | 38.2 |
| | AD-35783.1 | UUuAGCCUUUCuAAuACGGdTsdT | 52.8 |
| | AD-35789.1 | AUUuAGCCUUUCuAAuACGdTsdT | 63.7 |
| | AD-35795.1 | uAUUuAGCCUUUCuAAuACdTsdT | 30.4 |
| | AD-35801.1 | UUcAuAUUuAGCCUUUCuAdTsdT | 52.8 |
| | AD-35807.1 | AUUcAuAUUuAGCCUUUCUdTsdT | −35.3 |
| | AD-35813.1 | AAUUcAuAUUuAGCCUUUCdTsdT | 30.9 |
| | AD-35819.1 | UUGAUuAGAGGUGAUGGGGdTsdT | −17.0 |
| | AD-35825.1 | UUUGAUuAGAGGUGAUGGGdTsdT | 54.7 |
| | AD-35784.1 | AAcACCUCUUUGAUuAGAGdTsdT | 40.3 |
| | AD-35790.1 | AAcAGUGGACCcAAcACCUdTsdT | 20.7 |
| | AD-35796.1 | AAcAAcAGUGGACCcAAcAdTsdT | 27.5 |
| | AD-35802.1 | uAAcAAcAGUGGACCcAACdTsdT | 6.3 |
| | AD-35808.1 | AuAAcAAcAGUGGACCcAAdTsdT | −71.3 |
| | AD-35814.1 | AAGAuAAcAAcAGUGGACCdTsdT | 9.5 |
| | AD-35820.1 | AUCcAAGAuAAcAAcAGUGdTsdT | 24.9 |
| | AD-35826.1 | UUGGcAUCcAAGAuAAcAAdTsdT | 7.6 |
| | AD-35833.1 | AAAGUUGGcAUCcAAGAuAdTsdT | 46.2 |
| | AD-35839.1 | uAcAAAGUUGGcAUCcAAGdTsdT | 37.3 |
| | AD-35845.1 | UuAcAAAGUUGGcAUCcAAdTsdT | −31.6 |
| | AD-35851.1 | uAGGGGUcAuAGGCcAAGUdTsdT | −29.8 |
| | AD-35857.1 | uAuAGGGGUcAuAGGCcAAdTsdT | 10.1 |
| | AD-35863.1 | AuAuAGGGGUcAuAGGCcAdTsdT | 35.5 |
| | AD-35869.1 | AAuAuAGGGGUcAuAGGCCdTsdT | 54.1 |
| | AD-35828.1 | uAAuAuAGGGGUcAuAGGCdTsdT | 36.0 |
| | AD-35834.1 | UuAAuAuAGGGGUcAuAGGdTsdT | 28.0 |
| | AD-35840.1 | AAAGGGCUGCCUuAUGGUGdTsdT | 28.0 |
| | AD-35846.1 | uAAAGGGCUGCCUuAUGGUdTsdT | −20.3 |
| | AD-35852.1 | uAGGuAAAGGGCUGCCUuAdTsdT | −21.1 |
| | AD-35858.1 | uACCUGGAGUGGuAGGuAAdTsdT | −28.0 |
| | AD-35864.1 | AAGuACCUGGAGUGGuAGGdTsdT | 0.3 |
| | AD-35870.1 | UUGGUCcAGCUcAGGUUUGdTsdT | 30.0 |
| | AD-35829.1 | UUUGGUCcAGCUcAGGUUUdTsdT | 49.8 |
| | AD-35835.1 | UUGUUUGGUCcAGCUcAGGdTsdT | −4.7 |
| | AD-35841.1 | AUGGAGCcAcAGCUGGUUUdTsdT | 33.7 |
| | AD-35847.1 | AAAUGGAGCcAcAGCUGGUdTsdT | −456.5 |
| | AD-35853.1 | uAAAUGGAGCcAcAGCUGGdTsdT | 15.5 |
| | AD-35859.1 | UuAAAUGGAGCcAcAGCUGdTsdT | −18.6 |
| | AD-35865.1 | UUuAAAUGGAGCcAcAGCUdTsdT | 62.7 |
| | AD-35871.1 | AUUuAAAUGGAGCcAcAGCdTsdT | 41.9 |
| | AD-35830.1 | uAUUuAAAUGGAGCcAcAGdTsdT | 56.3 |
| | AD-35836.1 | UUGGUGUGGGuAUUuAAAUdTsdT | 29.0 |
| | AD-35842.1 | AUUGGUGUGGGuAUUuAAAdTsdT | 36.4 |
| | AD-35848.1 | UUUUGGAGCGcAuAGCCGAdTsdT | −41.1 |
| | AD-35854.1 | AUUUUGGAGCGcAuAGCCGdTsdT | −6.2 |
| | AD-35860.1 | UUGGGCUGUGGCUGcAUUUdTsdT | −6.2 |
| | AD-35866.1 | UUUGGGCUGUGGCUGcAUUdTsdT | 29.0 |
| | AD-35872.1 | AAUUUUGGGCUGUGGCUGCdTsdT | 41.5 |
| | AD-35831.1 | uAAUUUUGGGCUGUGGCUGdTsdT | 44.3 |
| | AD-35837.1 | AuAAUUUUGGGCUGUGGCUdTsdT | 23.9 |
| | AD-35843.1 | uACcAcAuAAUUUUGGGCUdTsdT | 37.3 |
| | AD-35849.1 | UuACcAcAuAAUUUUGGGCdTsdT | 12.6 |
| | AD-35855.1 | uAGUcAGCCUuACcAcAuAdTsdT | 21.2 |
| | AD-35861.1 | AuAGUcAGCCUuACcAcAUdTsdT | −23.7 |
| | AD-35867.1 | AAuAGUcAGCCUuACcAcAdTsdT | 50.5 |
| | AD-35873.1 | AAAuAGUcAGCCUuACcACdTsdT | 34.2 |
| | AD-35832.1 | uAAAuAGUcAGCCUuACcAdTsdT | 78.3 |
| | AD-35838.1 | uAcAuAAAuAGUcAGCCUUdTsdT | 41.6 |
| | AD-35844.1 | UUGuAcAuAAAuAGUcAGCdTsdT | 26.8 |
| | AD-35850.1 | AUUGuAcAuAAAuAGUcAGdTsdT | 66.3 |
| | AD-35856.1 | UUCUCUGAAUUGuAcAuAAdTsdT | 80.4 |
| | AD-35862.1 | AUUCUCUGAAUUGuAcAuAdTsdT | 43.0 |
| | AD-35868.1 | AAAUUCUCUGAAUUGuAcAdTsdT | 75.6 |
| | AD-35874.1 | uAAAUUCUCUGAAUUGuACdTsdT | 67.5 |

### Example 8

Transiently transfected siRNAs in DG44 suspension cultures show significant and long term interference for up to 18 days at concentrations as low as 0.1 nM.

RNA Interference of Suspension Cultures Grown at Different Temperatures:

GFP expressing CHO DG44 cells that are stably transfected with a CMV-GFP construct (Stratagene, Santa Clara, Calif.) were seeded at day 0 in wells of 96 well microtiter plates (at 2×10⁴ cells per well for 37° C. cells, and 10⁵ cells per well for 28° C. cells), and were transiently transfected with siRNAs against GFP at 0.1, 1, and 10 nM (formulated with LIPOFECTAMINE® RNAiMax reagent), also at day 0. GFP expression was measured fluorometrically; inhibition of expression (expressed as % of expression compared to RNAiMax only controls at the respective temperatures and times). Inhibition of expression was monitored for up to 18 days after the initial siRNA transfection.

Control Experiments:

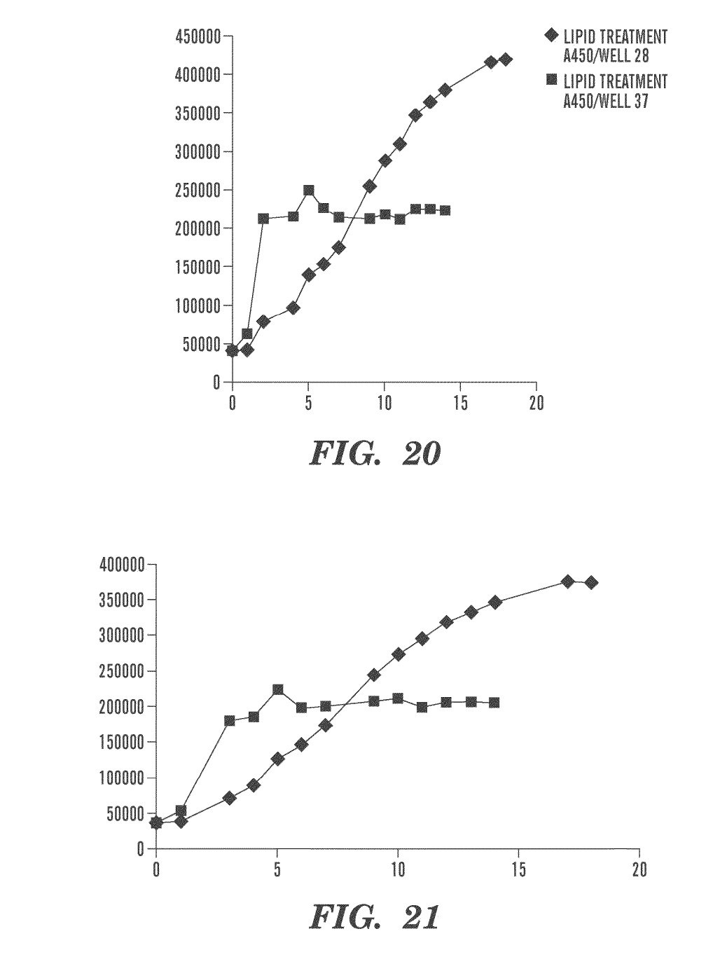

Expression of GFP in the CHO DG44 cells that were either untreated or RNAiMax only treated were monitored over time. The results are shown if FIG. 20 (untreated) and FIG. 21 (treated with lipid (RnaiMax only, no siRNAs). GFP is expressed over the course of the entire time period; however, expression of GFP in the 28° C. cells eventually became much higher, indicating continued protein expression, even in the absence of cell division (FIGS. 20 and 21).

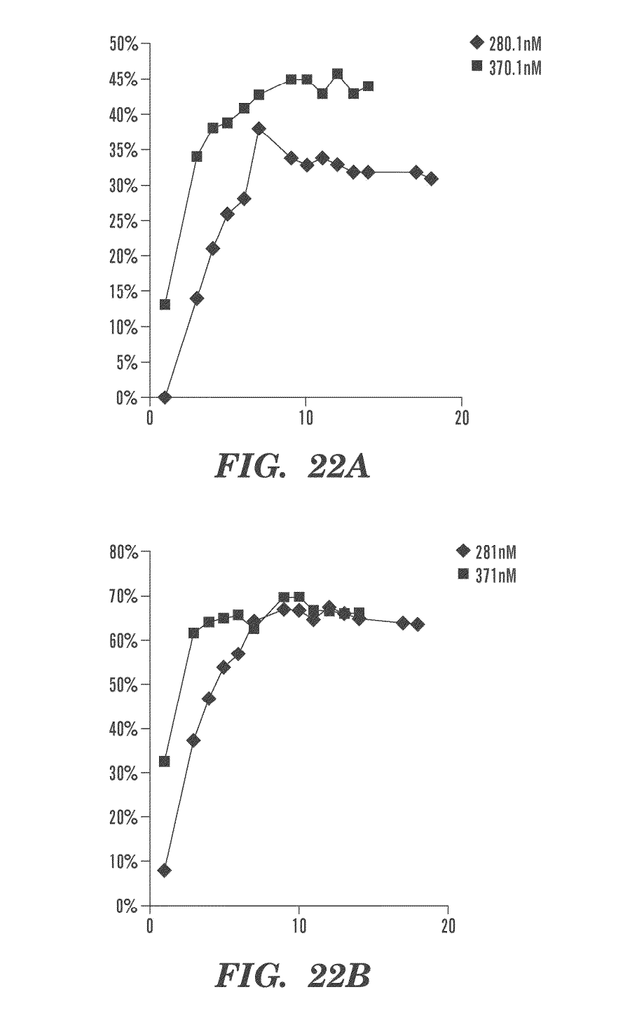

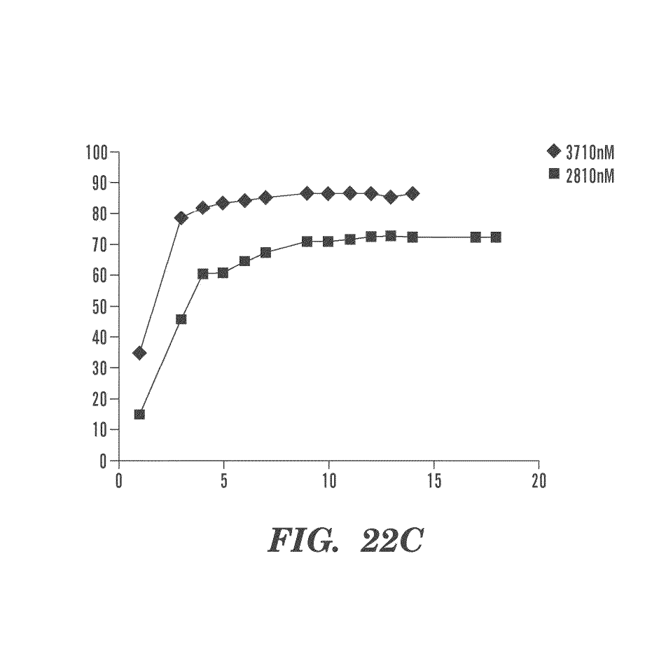

The lipid treated controls (FIG. 20) were used as controls for measuring efficacy of RNA interference. The graphs in FIG. 22A-22C show significant inhibition of expression of GFP at siRNA concentrations as low as 0.1 nM (FIG. 22A). Furthermore, inhibition of expression was maintained as long as the measurements were taken (i.e., in some cases, up to 18 days after initial expression)

### Example 13

### Scalable siRNA Uptake Protocol for CHO Cells Grown in a 40 L Bioreactor

As known to those of skill in the art liposome mediated delivery of siRNA using lipid polynucleotide carriers is commonly used in research applications, however, as described in PCT publication WO 2009/012173 (filed Jul. 11, 2008), the use of lipid polynucleotide carriers, e.g., common liposome transfection reagents, has been found to be detrimental when used in bioprocessing of protein. Polynucleotide carriers have been reported to be deleterious to the growth of host cells at the concentrations typically used presumably due to toxicity such that they impair the ability of host cells to produce the desired biological material on an industrial level. In addition polynucleotide carriers have been observed to cause adverse and unwanted changes in the phenotype of host cells, e.g., CHO cells, compromising the ability of the host cells to produce the biological product of interest. Accordingly, the artisan would expect that the use of such polynucleotide carriers would hinder a cells ability to produce a desired protein. Surprisingly, we have found, as described throughout herein, that RNA effector molecules (e.g., targeting BAX, BAC and/or LDH) can be delivered transiently to host cells in culture by using polynucleotide carriers (e.g., liposome mediated delivery) during the bioprocessing procedure in large scale cultures (e.g., 1 L and, e.g., 40 L) without detrimental effects on the cells under conditions tested on the cells, e.g., cell viability and density is maintained. Thus, large scale production of biological products can be done on an industrial scale using lipid reagents to facilitate RNA effector uptake in cells when they are in culture (e.g., suspension culture), for example, to result in effective transient modulation of gene expression that improve production of biological products (e.g., polypeptides).

Furthermore, we have studied various lipid compositions to identify efficient uptake enhancing reagents that promote efficient siRNA uptake into production cell lines with minimal impact on cell growth and viability. We had earlier demonstrated greater than 90% reduction in LDH activity (using siRNA directed against LDH) in 96-well plate cultures while screening a panel of quaternary cationic lipid formulations (data not shown). In this example, we show that siRNA formulated with P8 as an uptake inducer (see, e.g., Table 19) is better tolerated than commercial RNAiMax with respect to the respective formulations effect on cell density and cell viability in 50 ml cultures. We scaled up our cultures to a large scale bioreactor and found that using P8 formulated siRNA directed against LDH achieved 80%-90% reduction in LDH activity for 6 days with a single 1 nM dose. We then scaled up our cultures to 3 L and 40 L. We found that formulation P8 promoted efficient uptake of an siRNA directed against lactate dehydrogenase (LDH-A) and resulted in >90% of LDH reduction of LDH activity in CHO cells grown in either a 3 L or 40 L bioreactor. Surprisingly, in scale-up experiments comparing 3 L to 40 L cultures, there is perfect linearity of silencing efficiency. The results are shown herein.

### Materials/Methods

Formulation of Transfection Reagents:

Cationic lipid and colipids (e.g., cholesterol and DOPE) in chloroform were dried by a N₂ stream followed by vacuum-desiccation to remove residual organic solvent. The dried lipid film was hydrated using 10 mM HEPES buffer, pH 7.4 at 37° C. The formed liposomes were extruded to yield an average particle size of ˜200 nm.

Testing of Transfection Reagents on Plated GFP-HO Cells:

Nine different proprietary transfection formulations (see e.g., Table 19) and Lipofectamine RNAiMax (Invitrogen) were used to deliver 1 nM of a potent siRNA against GFP to a GFP-CHO cell line. RNAiMax was tested at 0.4 μL/mL and the nine formulations were used at 0.5, 1, and 2.5 μg/mL. Mixtures of transfection reagents and siRNA were made in black optical bottom 96 well plates and then cells were added. After 2 days, the relative GFP intensities were measured using a fluorescent plate reader.

Testing of Transfection Reagents on Suspended DG44 CHO Cells:

The three most active transfection agents (K8, L8 and P8) from the GFP-CHO testing were used to transfect suspended CHO cells. Aliquots of 5 μL, of 10 μM LDH-A siRNA were added to a tube and 500 μL CD DG44 media added to it. Transfection reagent was added to the mixture, the tube mixed by pipette aspiration and incubated at room temperature for 15 min. Then the mixture was added to 49.5 mL of media containing 200,000 cells/mL. The flask was incubated and shaken at 120 rpm for several days. LDH activity was measured by VetTest 8008 slide analyzer.

40 L Transfection:

DG44 cells were grown in Invitrogen CD DG44 media. To seed the 40 L bioreactor, cells were taken from four 1 L disposable bioreactors. The starting cell density in the 40 L of culture was 120,000 cells/mL. The bioreactor was allowed to equilibrate with the cells added for 1 hr prior to transfection. For transfection, 400 μL, of LDH-A siRNA (pair of SEQ ID NO:3152560 and NO:3152561) (100 uM stock solution) was added to 400 mL of media and mixed. Then 32 mL of 1 mg/mL P8 reagent was added and again mixed. This was allowed to incubate for 15 min at room temperature and then added to the 40 L bioreactor. Cell density and viability were measured using a Vi-Cell cell counter, and to determine the efficiency of transfection, LDH activity was measured using a VetTest 8008 slide analyzer.

### Results and Discussion

Evaluation of Nine Cationic Lipid Formulations for Uptake Efficiency in CHO Cells in Shake Flasks:

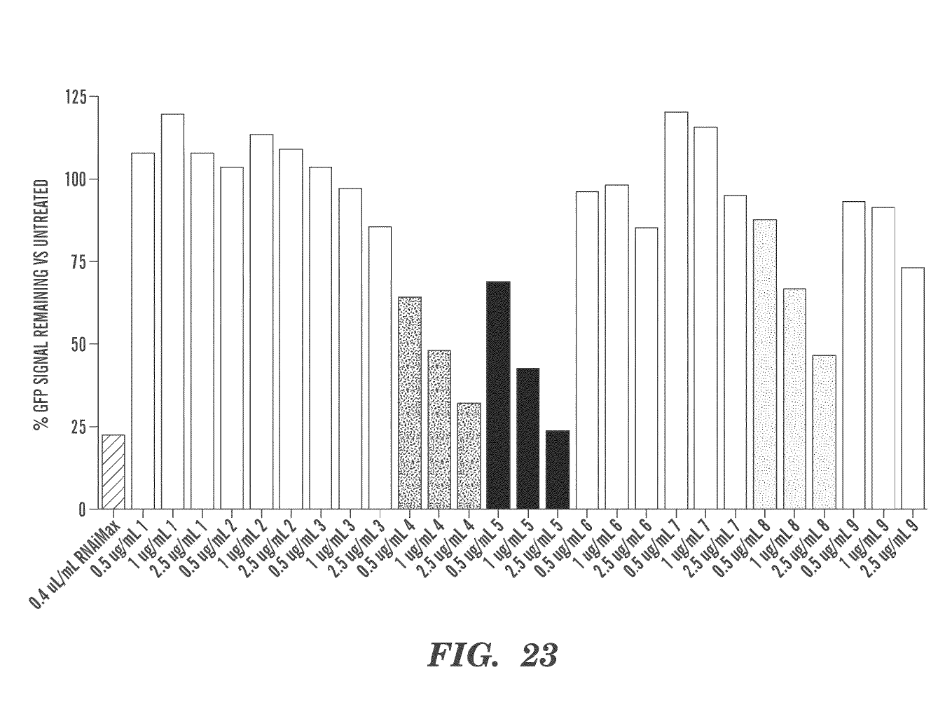

To gauge the effectiveness of the lipid formulations, they were used with a potent GFP siRNA in GFP-CHO cells. Compared with an effective concentration of LIPOFECTAMINE® RNAiMAx reagent, three compounds were active (FIG. 23). These formulations were designated K8, L8, and P8. No obvious cytotoxicity was observed at the concentrations tested of any formulation.

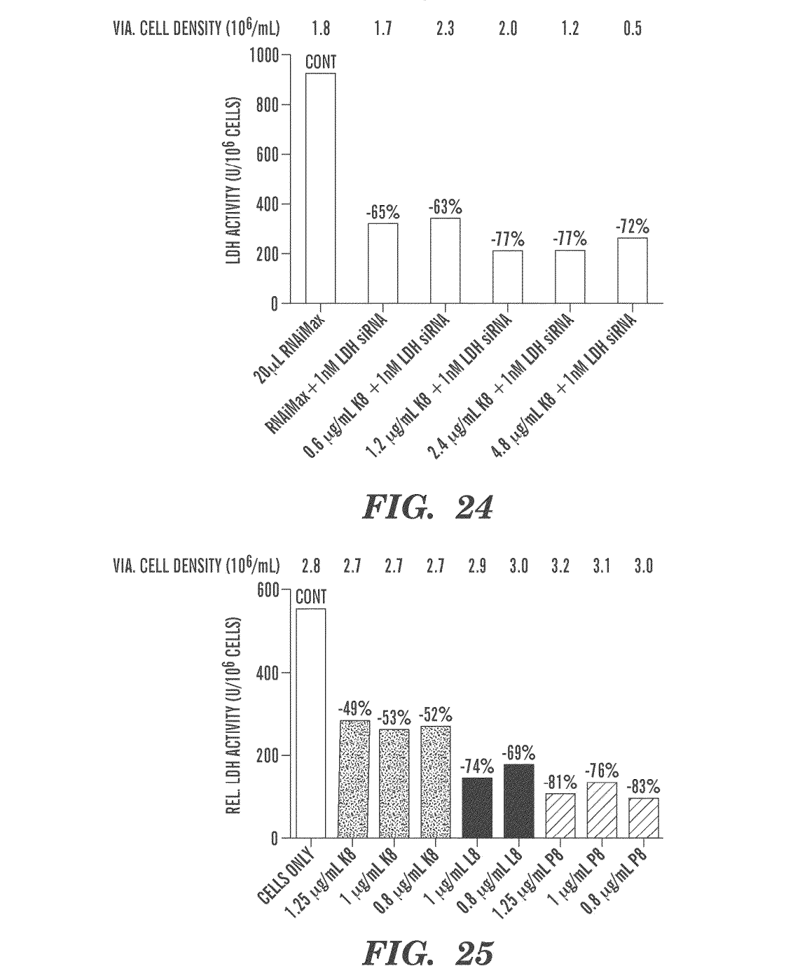

Because K8 was the most active formulation in the GFP-CHO cells, it was tested using DG44 CHO cells in 50 mL of culture in a 250 mL shake flask and a potent LDH siRNA. A range of K8 concentrations was tested along with an effective concentration of LIPOFECTAMINE® RNAiMAx transfection reagent. After 3 days, LDH activity was lower in cultures where K8 was used (FIG. 24). There was also a higher cell density in flasks that had 0.6 ng/mL or 1.2 ng/mL of K8 compared to RNAiMAx reagent. It appears that RNAiMAx reagent inhibited growth of CHO cell in suspension when compared to K8-treated cells. The highest concentrations of K8 reduced the cell density, even though the LDH activity was still reduced.

Because some transfection reagents didn not seem to have the same activity in shake flasks as in a 96-well plate, the three most active formulations were tested similarly in 50 mL of DG44 culture in 250 mL shake flasks. Surprisingly, formulation P8, which was only marginally active against GFP-CHO cells, performed the best using suspended DG44 cell culture (FIG. 25). After 5 days, 0.8 μg/mL of P8 resulted in the most LDH activity knockdown. Also, it is significant that the cell density in the presence of P8 was greater than or equal to control cells without transfection reagent added. P8 at a final concentration of 0.8 μg/mL has been used numerous times in smaller bioreactors and (data not shown) and was tested in a 40 L system.

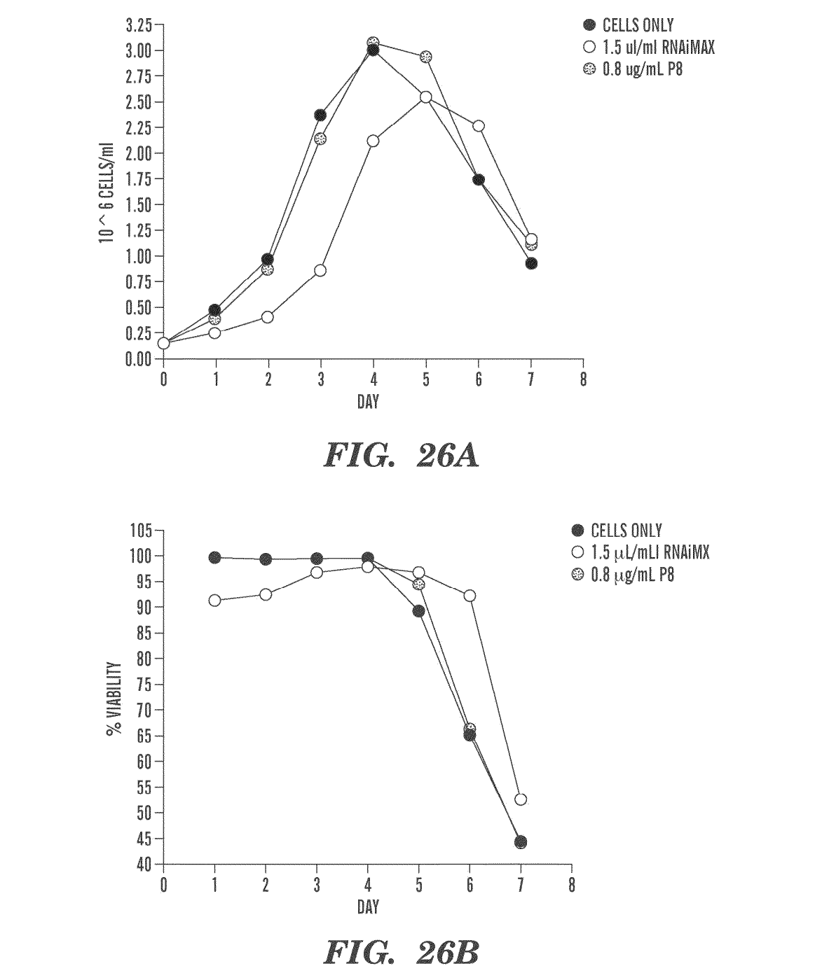

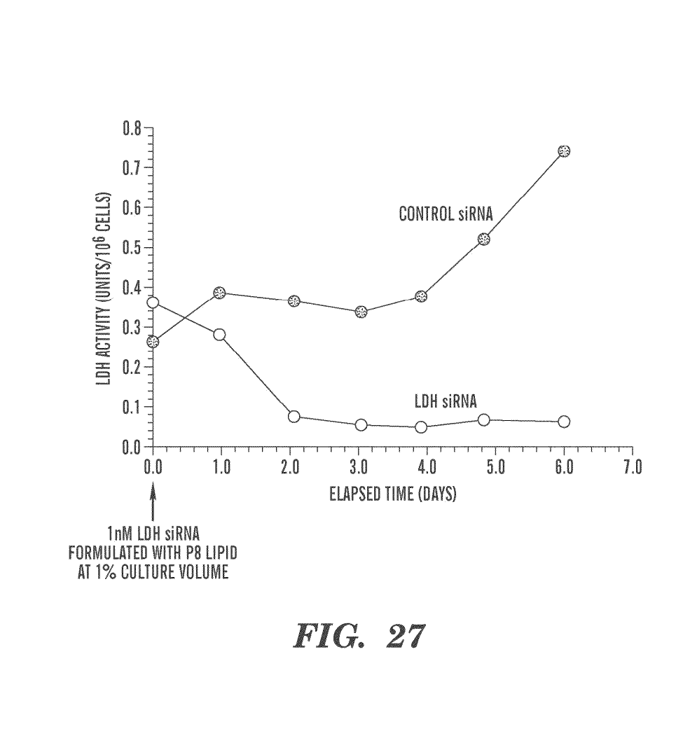

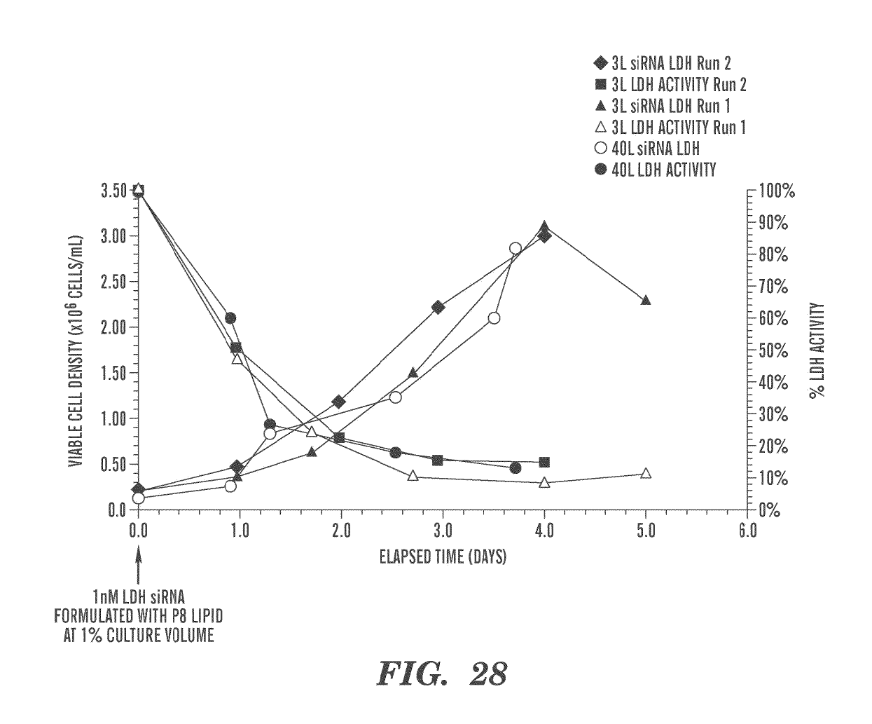

FIG. 26 shows cell density (FIG. 26A) or % cell viability (FIG. 26B) over time in suspension CHO cell 50 mL shake flasks using P8 formulation or commercial formulation RNAiMax at the recommended concentration. Lipid formulations were dosed onto cells at day 0. P8 was found to be better tolerated than commercial RNAiMax. FIG. 30 is a graph that shows that when using the P8 formulated siRNA directed against Lactate Dehydrogenase (LDH) achieves 80%-90% knockdown of LDH activity for 6 days with a single 1 nM dose in a 1 L bioreactor. Knockdown of LDH activity was found to be durable, with effects lasting over 6 days.

Evaluation of Cationic Lipid Formulation P8 for Uptake Efficiency in a 3 L Vs 40 L Bioreactor:

FIG. 28 shows the results of a single dose of an 1 nM LDH siRNA formulated with P8 lipid on viable cell density and % LDH activity over an elapsed time of 6 days in 3 L and 40 L cultures. Surprisingly, in scale-up experiments comparing 3 L to 40 L cultures, there is perfect linearity of silencing efficiency indicating success at even larger scales. Multiple dose protocols can be used to extend the duration of effect.

Evaluation of Cationic Lipid Formulation P8 for Uptake Efficiency in a 40 L Bioreactor:

After seeding the 40 L bioreactor, the cells generally grew with a doubling time of approximately 24 hr and the cell viability was over 98% (FIG. 26B). The cells reached a peak concentration of 3.1×10⁶ cells/mL at day 5 and then began to decline. As expected in this unfed batch culture, by day 6 the cells were in decline.

The LDH activity of the siRNA treated cells was reduced as the cells were growing following seeding and transfection. The LDH activity was reduced ˜80% even as the cells had doubled over 3 times (FIG. 30). There was diminished LDH activity through the entire experiment. Based on the significantly diminished LDH activity, the transfection was successful with no detectable toxicity in the CHO cells.

These experiments show that transfection of cells in culture with siRNAs can work in the large volumes necessary for biological production.

### Example 14

### Use of RNA Effectors to Titrate Expression of Target Genes

Unlike cells with stably transfected shRNA, use of dsRNA molecules allows modulation of expression of practically any target gene within a host cell without the need for cell engineering. In addition, as mentioned previously, cells with constitutively inhibited target genes may not grow well and may display unwanted characteristics (e.g., need for special growth media or other growth conditions, increased rate of mutation, etc). Having the ability to modulate expression of a target gene at the desired point during growth of a cell or production of a biologic is therefore highly desirable.

Yet another advantage of using RNA effector molecules such as dsRNA agents that do not rely on stable transfection is the potential ability to fine-tune expression of a given target gene. In some cases it may be important to regulate expression of a target gene such that its expression level is only moderately altered (e.g., decreased by ˜50% from the untreated state) so as to avoid unwanted phenotypes or to improve the quality of biologic production. As such, we performed experiments to find conditions in which expression of a given target gene could be titrated.

On day 0, CHO DG-44 cells grown in CD DG44 media (Invitrogen), were transfected with dsRNA targeting the LDHA gene (as described herein; see e.g., Table 62) at 0 nM, 10 pM, 50 pM, 100 pM, 500 pM, 1 nM and 5 nM (final concentrations in 25 mL of culture), in a formulation containing the Lipid P, in formulation 8 (i.e., formulation “P8”; see Table 19) in a 500 μL volume. The dsRNA duplex used has an apparent EC₅₀ of ˜50 pM under similar conditions. After transfection, cells were added to a flask containing 24.5 mL of media (at a cell density of 200,000 cells/mL) and grown at 37° C. After 3 days, LDH activity was measured and normalized to cell density.

The LDH activity is shown in Table 62 below:

TABLE 62
LDH activity

| | | Via | LDH | (LDH activity/ | % |
|---|---|---|---|---|---|
| | dsRNA | den- | activ- | 106 cells)* | knock- |
| Flask | concentration | sity | ity | mL dil. | down |
| 1 | 0 LDH siRNA | 2.11 | 1489 | 776.3 | |
| 2 | 10 pM LDH siRNA | 2.08 | 1248 | 660.0 | 15.0 |
| 3 | 50 pM LDH siRNA | 2.08 | 754 | 398.8 | 48.6 |
| 4 | 100 pM LDH siRNA | 2.22 | 560 | 277.5 | 64.3 |
| 5 | 500 pM LDH siRNA | 2.22 | 335 | 166.0 | 78.6 |
| 6 | 1 nM LDH siRNA | 2.16 | 335 | 170.6 | 78.0 |
| 7 | 5 nM LDH siRNA | 2.21 | 363 | 180.7 | 76.7 |

The results show that LDH activity can be modulated to a range between 15% to greater than 75% inhibition by titrating the concentration of dsRNA. Therefore, use of RNA effector molecules such as the dsRNAs shown herein can be used to achieve a desired expression level of the target gene. In addition, based on earlier experiments (not shown), cells treated at concentrations in which partial inhibition is achieved (for example, at 10-100 pM) are expected to recover from RNA interference more rapidly than those treated at higher concentrations. As such, where it is desirable to have cells recover from inhibition of a target gene faster (i.e., inhibition of gene expression will persist for a shorter period of time), then one can provide a lower concentration of RNA effector molecule (e.g., 3× of the apparent EC₅₀ or less, for example 2× the apparent EC50, 1× the apparent EC50, etc).

The following tables exemplify target genes and siRNA sequences useful with the methods and compositions described herein.

TABLE 51
Target genes

| | | siRNA |
|---|---|---|
| Target | Description | SEQ ID NOs |
| 15-lipoxygenase-1 | arachidonate lipoxygenase 3 | 2480018-2480362 |
| Ago2 | eukaryotic translation initiation factor 2C, 2 | 255154-255411 |
| Ago3 | eukaryotic translation initiation factor 2C, 3 | 3103755-3103973 |
| Ago4 | eukaryotic translation initiation factor 2C, 4 | 1326374-1326705 |
| APAFI | apoptotic peptidase activating factor 1 | 2262408-2262743 |
| ApoE | apolipoprotein E | 3172384-3172483 |
| asparagine deamidase | N-terminal Asn amidase | 1999410-1999756 |
| glutamine deamidase | WDYHV motif containing 1; aka Protein | 2478078-2478376 |
| | NH2-terminal glutamine deamidase | |
| ATF4 | activating transcription factor 4 | 1552067-1552460 |
| ATF6 | activating transcription factor 6 | 570138-570498 |
| ATF6P | activating transcription factor 6 beta | 471680-472070 |
| B4GalT1 | UDP-Gal: βGlcNAc β1,4-galactosyltransferase, | 2528454-2528763 |
| | polypeptide 1 | additional |
| | | galactosyl & |
| | | galactosaminyl- |
| | | transferases |
| | | elsewhere herein |
| BAD | BCL2-associated agonist of cell death | 3049436-3049721 |
| BAG-1 | BCL2-associated athanogene | 1683576-1683895 |
| Bcl-w | BCL2-like 2 | 477629-477999 |
| Bcl-xL | BCL2-like 1 | 728838-729216 |
| Bid | BH3 interacting domain death agonist | 2582517-2582823 |
| Bik | BCL2-interacting killer (apoptosis-inducing) | 2899985-2900289 |
| BIM/BimL | BCL2-like 11 (apoptosis facilitator) | 1960442-1960764 |
| BNIP3 | BCL2/adenovirus E1B 19 kDa interacting protein 3 | 1740754-1741152 |
| calnexin | calnexin | 61559-61785 |
| calreticulin | Calreticulin | 895691-896051 |
| CASP2 | Caspase 2 | 2718675-2719039 |
| CASP3 | Caspase 3 | 1924836-1925195 |
| CASP6 | Caspase 6 | 2408466-2408843 |
| CASP7 | Caspase 7 | 2301618-2301960 |
| CASP8 | Caspase 8 | 2995593-2995870 |
| CASP9 | Caspase 9 | 1412589-1412860 |
| CCNA2/Cyclin A2 | cyclin A2 | 1151948-1152332 |
| CCNB1/Cyclin B1 | Cyclin B1 | 1298863-1299236 |
| CCNB2/Cyclin B2 | Cyclin B2 | 1489394-1489722 |
| CCND1/Cyclin D1 | cyclin D1 | 139242-139629 |
| CCND2/Cyclin D2 | cyclin D2 | 960077-960401 |
| CCND3/Cyclin D3 | Cyclin D3 | 1040554-1040910 |
| CCNE1/Cyclin E1 | cyclin E1 | 1980613-1981009 |
| CCNE2/Cyclin E2 | Cyclin E2 | 2904183-2904530 |
| CDK2 | cyclin-dependent kinase 2 | 1193336-1193684 |
| CDK4 | cyclin-dependent kinase 4 | 1609522-1609852 |
| Cmas | cytidine monophosphate N-acetylneuraminic | 1633101-1633406 |
| | acid synthetase | |
| Cofilin (CFL1) | Cofilin | 1914036-1914356 |
| cytochrome P4502E1 | | |
| cytoplasmic actin | capping protein (actin filament) muscle Z-line, | 235917-236159 |
| capping protein (CapZ) | α1 | |
| dihydrofolate reductase | | 1739672-1740059 |
| Eri1 | exoribonuclease 1 | 3244117-3244216 |
| Ezrin (VIL2) | Ezrin | 339220-339540 |
| fucosyltransferase/ | Fucosyltransferases | FUT8 dsRNA: |
| FUT8 | | 209841-210227 |
| | | additional seqs |
| | | elsewhere herein |
| GLUT1 | solute carrier family 2 (facilitated glucose | 438155-438490 |
| | transporter), member 1 | additional seqs |
| | | elsewhere herein |
| glutaminase | | 105170-105438 |
| GMDS | GDP mannose dehydratase | 1688202-1688519 |
| Gne | glucosamine (UDP-N-acetyl)-2-epimerase/ | 2073971-2074368 |
| | N-acetylmannosamine kinase; | |
| | UDP-N-acetylglucosamine-2-epimerase/ | |
| | N-acetylmannosamine kinase | |
| GRP94 | heat shock protein 90 kDa β(Grp94), member 1 | 180574-180954 |
| HR | Hairless | 1110794-1111079 |
| Hsp40 | DnaJ (Hsp40) homolog | dsRNA sequences |
| | | targeting Hsp40 |
| | | elsewhere herein |
| interferon receptor | IFNAR1 | 2436536-2436863 |
| IRE1 | endoplasmic reticulum (ER) to nucleus signaling 1 | 3179284-3179383 |
| Laminin A | | 5: 48814-49139 |
| | | 2: 2954307-2954650 |
| | | 3: 3160721-3160820 |
| lysosomal V-type | | For sequences of |
| ATPase | | the various |
| | | subunits please see |
| | | table below |
| Mcl-1 | myeloid cell leukemia sequence 1 (BCL2-related) | 312684-312913 |
| N-acetylgalactosaminy- | | 2876241-2876595, |
| transferase T-4 | | see also, e.g., |
| | | Table 6 |
| NAD(p)H oxidase | | See table |
| | | elsewhere herein |
| | | for cytochrome |
| | | reductases |
| NADH cytochrome | | |
| b5 reductase | | |
| NADPH cytochrome | | |
| c2 reductase | | |
| NAPH cytochrome | | |
| c reductase | | |
| B4GalT6. | and UDP-Gal: βGlcNAc β1,4- | 3154201-3154224 |
| | galactosyltransferase, polypeptide 6 | (sense) and |
| | | 3154225-3154248 |
| P10 | S100 calcium binding protein A10 (calpactin) | 3013998-3014274 |
| p115 | USO1 vesicle docking protein homolog (yeast) | 89340-89737 |
| P14ARF/p16INK4a | cyclin-dependent kinase inhibitor 2A | 2B: 2895015-2895359 |
| | (melanoma, p16, inhibits CDK4) | 2C: 1969649-1970047 |
| | | 2D: 1990790-1991181 |
| P21 | cyclin-dependent kinase inhibitor 1A (p21, | 2659502-2659871 |
| | Cip1) | |
| P27 | proteasome (prosome, macropain) 26S subunit, | 3199397-3199496 |
| | non-ATPase, 9 | |
| p53 | tumor protein p53; | 1649857-1650157 |
| | transformation related protein p53 | |
| P57 | cyclin-dependent kinase inhibitor 1C (p57, | 1A: 2659502-2659871 |
| | Kip2) | 1B: 2731076-2731440 |
| peptidyl prolyl | peptidylprolyl isomerase | 1074139-1074475, |
| isomerase | | 1085316-1085607, |
| | | 1127061-1127426, |
| | | 1649170-1649515, |
| | | 1780604-1780923, |
| | | 2197146-2197532, |
| | | 2253978-2254373, |
| | | 2261765-2262058, |
| | | 2275330-2275633, |
| | | 2579547-2579908, |
| | | 2857424-2857802, |
| | | 3136158-3136181, |
| | | 3262205-3262304 |
| PERK | eukaryotic translation initiation factor | 1396283-1396617; |
| | 2-kinase 3 | Kinase 4: |
| | | 582987-583297; |
| | | Kinase 1: |
| | | 1037660-1038052 |
| peroxidase | | siRNAs targeting |
| | | Glutathione |
| | | peroxidases |
| | | include: |
| | | 2439217-2439612 |
| | | 2560559-2560895 |
| | | 2703865-2704225 |
| | | 3151589-3151685 |
| | | See table below |
| | | for enzymes |
| | | possessing |
| | | peroxidase activity |
| phosphatidylinositol- | phosphatase and tensin homolog | 69091-69404 |
| 3,4,5-trisphosphate | | |
| 3-phosphatase (PTEN) | | |
| protein disulfide | | These siRNAs |
| isomerase | | target genes that |
| | | have protein |
| | | disulfide |
| | | isomerase activity: |
| | | 72748-72996 |
| | | 335875-336225 |
| | | 488676-489039 |
| | | 774355-774677 |
| | | 898511-898822 |
| | | 966735-967056 |
| protein | protein O-fucosyltransferase 1 | 2321807-2322122 |
| O-fucosyltransferase | | |
| PUMA | BCL2 binding component 3 | 1712045-1712425 |
| SLC35A1 | solute carrier family 35 (CMP-sialic acid | 3154345-3154368; |
| | transporter), member 1 | 1367952-1368265 |
| ST3 β-galactoside α- | | 681105-681454 |
| 2,3-sialyltransferase 1 | | |
| ST3 β-galactoside α- | | 1435989-1436317 |
| 2,3-sialyltransferase 2. | | |
| ST3 β-galactoside α- | | 1131123-1131445 |
| 2,3-sialyltransferase 3 | | |
| ST3 β-galactoside α- | | 707535-707870 |
| 2,3-sialyltransferase 4 | | |
| ST3 β-galactoside α- | | 1155324-1155711 |
| 2,3-sialyltransferase 5 | | |
| ST6 (a-N-acetyl- | | 1391079-1391449 |
| neuraminy 1-2,3-β- | | |
| galactosyl-1,3)-N- | | |
| acetylgalactosaminide α- | | |
| 2,6-sialyltransferase 6 | | |
| TSTA3 | tissue specific transplantation antigen P35B | 1839578-1839937 |
| xanthine oxidase (XO) | Aka xanthine dehydrogenase | 374846-375216 |
| xylose transferase | Xylosyltansferase II | 1554774-1555054 |
| α galactosidase | | 1600968-1601288 |
| β-galactosidase | | 690601-690989 |

TABLE 52
GLUTs (glucose transporters)

| SEQ | | siRNA |
|---|---|---|
| ID NO: | Description | SEQ ID NOs: |
| 1375 | solute carrier family 2 (facilitated | 438155-438490 |
| | glucose transporter), member 1 | |
| 6869 | solute carrier family 2, (facilitated | 2325698-2325997 |
| | glucose transporter), member 8 | |
| 7909 | solute carrier family 2 (facilitated | 2669929-2670303 |
| | glucose transporter), member 13 | |

TABLE 53
Fucosyltransferases

| SEQ | | | siRNA |
|---|---|---|---|
| ID NO: | consL | Description | SEQ ID NOs: |
| 676 | 2680 | fucosyltransferase 8 | 209841-210227 |
| 2783 | 1861 | protein O-fucosyltransferase 2 | 916726-917035 |
| 6857 | 913 | protein O-fucosyltransferase 1 | 2321807-2322122 |
| 8126 | 593 | fucosyltransferase 11 | 2740650-2740952 |

TABLE 54
DnaJ (Hsp40) homologs

| SEQ | | | Avg | siRNA |
|---|---|---|---|---|
| ID NO: | consL | Description | Coverage | SEQ ID NOs: |
| 1932 | 2102 | Subfamily A, member 1 | 18.764 | 628385-628725 |
| 893 | 2541 | Subfamily A, member 2 | 15.853 | 276519-276904 |
| 1925 | 2104 | Subfamily A, member 3 | 15.15 | 625909-626254 |
| 3157871 | 528 | Subfamily A, member 4 | 0.656 | 3215391-3215490 |
| 2076 | 2052 | Subfamily B, member 1 | 9.75 | 677203-677558 |
| 5350 | 1247 | Subfamily B, member 11 | 17.061 | 1784585-1784897 |
| 5347 | 1248 | Subfamily B, member 12 | 3.209 | 1783440-1783810 |
| 9545 | 230 | Subfamily B, member 13 | 0.22 | 3133435-3133598 |
| 3157418 | 441 | Subfamily B, member 14 | 0.238 | 3228617-3228716 |
| 4158 | 1511 | Subfamily B, member 2 | 5.045 | 1381610-1381931 |
| 3158137 | 878 | Subfamily B, member 3 | 1.052 | 3283549-3283648 |
| 5405 | 1236 | Subfamily B, member 4 | 1.568 | 1804161-1804465 |
| 8128 | 593 | Subfamily B, member 5 | 0.47 | 2741242-2741540 |
| 2619 | 1902 | Subfamily B, member 6 | 14.116 | 860762-861101 |
| 5149 | 1289 | Subfamily B, member 9 | 0.929 | 1715305-1715623 |
| 4159 | 1510 | Subfamily C, member 1 | 3.933 | 1381932-1382211 |
| 546 | 2787 | Subfamily C, member 10 | 22.023 | 171304-171555 |
| 1143 | 2405 | Subfamily C, member 11 | 15.429 | 360296-360688 |
| 3157835 | 1640 | Subfamily C, member 13 | 0.983 | 3240717-3240816 |
| 412 | 2946 | Subfamily C, member 14 | 7.271 | 133746-134002 |
| 9442 | 267 | Subfamily C, member 15 | 0.656 | 3117145-3117332 |
| 1960 | 2089 | Subfamily C, member 16 | 1.225 | 637892-638209 |
| 6631 | 962 | Subfamily C, member 17 | 1.346 | 2243108-2243387 |
| 7277 | 817 | Subfamily C, member 18 | 0.36 | 2460206-2460591 |
| 9036 | 381 | Subfamily C, member 19 | 1.461 | 3027351-3027657 |
| 2513 | 1930 | Subfamily C, member 2 | 34.4 | 825067-825402 |
| 2721 | 1878 | Subfamily C, member 21 | 8.299 | 895321-895690 |
| 5660 | 1176 | Subfamily C, member 22 | 4.382 | 1893667-1894030 |
| 8661 | 464 | Subfamily C, member 24 | 2.068 | 2917681-2918006 |
| 6150 | 1068 | Subfamily C, member 25 | 0.929 | 2072060-2072449 |
| 8171 | 583 | Subfamily C, member 27 | 0.773 | 2754733-2755101 |
| 3157934 | 1241 | Subfamily C, member 28 | 2.604 | 3271096-3271195 |
| 1054 | 2449 | Subfamily C, member 3 | 10.89 | 330430-330812 |
| 6648 | 959 | Subfamily C, member 30 | 1.456 | 2249119-2249439 |
| 7348 | 800 | Subfamily C, member 4 | 4.236 | 2483678-2484063 |
| 2403 | 1958 | Subfamily C, member 5 | 5.417 | 787385-787676 |
| 9017 | 388 | Subfamily C, member 6 | 0.078 | 3022706-3022949 |
| 3188 | 1749 | Subfamily C, member 7 | 20.562 | 1055444-1055806 |
| 5052 | 1312 | Subfamily C, member 8 | 41.714 | 1682260-1682641 |
| 7247 | 827 | Subfamily C, member 9 | 4.989 | 2450765-2451126 |

TABLE 55
Heat Shock proteins

| SEQ | | | Avg | siRNA |
|---|---|---|---|---|
| ID NO: | consL | Description | Cov | SEQ ID NOs: |
| 444 | 2900 | heat shock protein 4 | 15.95 | 142820-143094 |
| 476 | 2865 | heat shock 105 kDa/110 kDa protein 1 | 19.863 | 151195-151420 |
| 485 | 2858 | AHA1, activator of heat shock protein | 16.103 | 153831-154084 |
| | | ATPase homolog 2 (yeast) | | |
| 579 | 2758 | heat shock protein 90, beta (Grp94), member 1 | 606.207 | 180574-180954 |
| 594 | 2744 | heat shock protein 90, alpha (cytosolic), class | 93.844 | 184698-184927 |
| | | A member 1 | | |
| 827 | 2572 | heat shock protein 9 | 28.56 | 255926-256325 |
| 941 | 2519 | heat shock protein 5 | 729.81 | 292590-292837 |
| 977 | 2496 | heat shock protein 90 alpha (cytosolic), class | 609.471 | 304274-304591 |
| | | B member 1 | | |
| 1543 | 2232 | heat shock protein 1 (chaperonin) | 134.366 | 494743-495086 |
| 2029 | 2068 | heat shock protein 8 | 891.015 | 660889-661277 |
| 2272 | 1990 | heat shock factor 2 | 2.598 | 743398-743788 |
| 2756 | 1869 | heat shock factor 1 | 25.227 | 907582-907889 |
| 2974 | 1807 | heat shock protein 2 | 5.538 | 982428-982785 |
| 3063 | 1776 | heat shock protein 8 | 38.69 | 1012333-1012621 |
| 3765 | 1608 | heat shock protein 14 | 20.386 | 1250279-1250587 |
| 4038 | 1541 | heat shock protein 70 family, member 13 | 2.835 | 1341514-1341853 |
| 4337 | 1473 | HSPA (heat shock 70 kDa) binding protein, | 11.687 | 1441933-1442264 |
| | | cytoplasmic cochaperone 1 | | |
| 5002 | 1323 | AHA1, activator of heat shock protein | 93.621 | 1665415-1665746 |
| | | ATPase homolog 1 (yeast) | | |
| 5756 | 1155 | heat shock factor binding protein 1 | 28.266 | 1928608-1928970 |
| 7697 | 715 | heat shock protein, -crystallin-related, B6 | 1.268 | 2598077-2598438 |
| 8336 | 539 | heat shock protein 1 | 3.124 | 2809108-2809434 |
| 8405 | 517 | heat shock protein 1 (chaperonin 10) | 4.477 | 2833031-2833420 |
| 9679 | 173 | heat shock protein 1B | 0.091 | 3147029-3147080 |

TABLE 56
Lysosomal V-type ATPase subunits

| SEQ | | siRNA |
|---|---|---|
| ID NO: | Description | SEQ ID NOs: |
| 198 | ATPase, H+ transporting, lysosomal V1 subunit A | 71796-72111 |
| 1027 | ATPase, H+ transporting, lysosomal V1 subunit B2 | 321673-321927 |
| 1796 | ATPase, H+ transporting, lysosomal accessory protein 2 | 582376-582610 |
| 2296 | ATPase, H+ transporting, lysosomal V0 subunit A1 | 751583-751949 |
| 2532 | ATPase, H+ transporting, lysosomal accessory protein 1 | 831609-831895 |
| 2762 | ATPase, H+ transporting, lysosomal V1 subunit H | 909697-910010 |
| 3329 | T-cell, immune regulator 1, ATPase, H+ transporting, | 1103103-1103418 |
| | lysosomal V0 protein A3 | |
| 4324 | ATPase, H+ transporting, lysosomal V0 subunit D1 | 1437602-1437944 |
| 4347 | ATPase, H+ transporting, lysosomal V0 subunit A2 | 1445278-1445615 |
| 5454 | ATPase, H+ transporting, lysosomal V1 subunit E1 | 1821367-1821755 |
| 5620 | ATPase, H+ transporting, lysosomal V1 subunit D | 1879531-1879860 |
| 5788 | ATPase, H+ transporting, lysosomal V0 subunit C | 1940302-1940675 |
| 5816 | ATPase, H+ transporting, lysosomal V1 subunit C1 | 1950210-1950528 |
| 6117 | ATPase, H+ transporting, lysosomal V1 subunit G1 | 2059770-2060150 |
| 6486 | ATPase, H+ transporting, lysosomal V0 subunit B | 2192145-2192538 |
| 7910 | ATPase, H+ transporting, lysosomal V0 subunit E2 | 2670304-2670626 |
| 7976 | ATPase, H+ transporting, lysosomal V1 subunit F | 2692263-2692620 |
| 7987 | ATPase, H+ transporting, lysosomal V0 subunit E | 2695797-2696168 |
| 8582 | ATPase, H+ transporting, lysosomal V1 subunit G2 | 2890746-2891087 |
| 3157707 | ATPase, H+ transporting, lysosomal V0 subunit D2 | 3281849-3281948 |

TABLE 57
Peroxidase

| SEQ | | | Avg | siRNA SEQ |
|---|---|---|---|---|
| ID NO: | consL | Description | Cov | ID NOs: |
| 442 | 2901 | heterogeneous nuclear ribonucleoprotein L-like | 11.591 | 142215-142508 |
| 1706 | 2173 | catalase | 18.084 | 551058-551444 |
| 3107 | 1768 | prostaglandin-endoperoxide synthase 2 | 0.699 | 1027449-1027832 |
| 6122 | 1074 | peroxiredoxin 3 | 15.819 | 2061664-2062027 |
| 6608 | 967 | peroxiredoxin 4 | 81.791 | 2235293-2235671 |
| 6741 | 937 | peroxiredoxin 6 | 9.666 | 2281128-2281515 |
| 6816 | 921 | peroxidasin homolog (Drosophila) | 0.334 | 2307638-2308007 |
| 7213 | 835 | glutathione peroxidase 1 | 10.976 | 2439217-2439612 |
| 7386 | 792 | peroxiredoxin 1 | 1.522 | 2496217-2496481 |
| 7582 | 743 | glutathione peroxidase 4 | 73.452 | 2560559-2560895 |
| 7749 | 702 | peroxiredoxin 2 | 15.903 | 2616024-2616366 |
| 8011 | 630 | glutathione peroxidase 8 (putative) | 15.42 | 2703865-2704225 |
| 8179 | 582 | peroxiredoxin 5 | 3.766 | 2757414-2757689 |
| 8565 | 482 | glutathione S-transferase kappa 1 | 1.46 | 2885542-2885890 |
| 8687 | 461 | iodotyrosine deiodinase | 0.299 | 2926039-2926366 |
| 9756 | 131 | glutathione peroxidase 3 | 0.087 | 3151589-3151685 |

TABLE 58
Protein Disulfide Isomerase Activity

| SEQ | | | Avg | siRNA |
|---|---|---|---|---|
| ID NO: | consL | Description | Cov | SEQ ID NOs: |
| 201 | 3342 | thioredoxin-related transmembrane protein 3 | 6.308 | 72748-72996 |
| 1071 | 2440 | prolyl 4-hydroxylase, beta polypeptide | 262.952 | 335875-336225 |
| 1525 | 2239 | protein disulfide isomerase associated 4 | 31.944 | 488676-489039 |
| 2364 | 1967 | protein disulfide isomerase associated 3 | 173.819 | 774355-774677 |
| 2730 | 1874 | protein disulfide isomerase associated 6 | 699.725 | 898511-898822 |
| 2929 | 1822 | protein disulfide isomerase associated 5 | 42.884 | 966735-967056 |

TABLE 59
Signal Recognition Particle

| SEQ | | | Avg | siRNA |
|---|---|---|---|---|
| ID NO: | consL | Description | Cov | SEQ ID NOs: |
| 348 | 3031 | signal recognition particle 72 | 13.053 | 115319-115586 |
| 498 | 2844 | signal recognition particle receptor (docking protein) | 23.636 | 157648-157932 |
| 1200 | 2382 | signal recognition particle 68 | 40.31 | 379331-379670 |
| 1535 | 2235 | signal recognition particle 54a | 4.713 | 492211-492502 |
| 2108 | 2042 | signal recognition particle 54b | 7.508 | 687895-687922 |
| 3277 | 1725 | signal recognition particle 54C | 3.004 | 1085608-1085800 |
| 6222 | 1053 | signal recognition particle 9 | 6.194 | 2097989-2098388 |
| 6901 | 903 | signal recognition particle receptor, B subunit | 8.479 | 2335474-2335804 |
| 7846 | 677 | signal recognition particle 14 | 2.01 | 2648705-2649066 |
| 9140 | 355 | signal recognition particle 19 | 0.4 | 3053860-3054133 |
| 8427 | 513 | retinitis pigmentosa 9 (human) | 0.65 | 2840748-2841112 |

TABLE 60
Example kinase targets

| SEQ | | siRNA |
|---|---|---|
| ID NO: | Description | SEQ ID NOs: |
| 2 | TAO kinase 1 | 10148-10532 |
| 16 | homeodomain interacting protein kinase 1 | 14439-14801 |
| 26 | dual-specificity tyrosine-(Y)-phosphorylation | 17461-17750 |
| | regulated kinase 1a | |
| 67 | casein kinase 2, alpha 1 polypeptide | 30901-31248 |
| 74 | mitogen-activated protein kinase kinase kinase | 33333-33668 |
| | kinase 4 | |
| 80 | Rho-associated coiled-coil containing protein kinase 2 | 35242-35563 |
| 92 | calcium/calmodulin-dependent serine protein kinase | 39068-39431 |
| | (MAGUK family) | |
| 105 | cDNA sequence BC033915 | 43314-43658 |
| 131 | mitogen-activated protein kinase 9 | 51635-51907 |
| 135 | Braf transforming gene | 52754-53026 |
| 153 | serine/arginine-rich protein specific kinase 2 | 57998-58262 |
| 160 | ribosomal protein S6 kinase, polypeptide 1 | 60208-60510 |
| 199 | protein kinase C, alpha | 72112-72439 |
| 211 | AP2 associated kinase 1 | 75589-75893 |
| 215 | AXL receptor tyrosine kinase | 76768-77080 |
| 249 | discoidin domain receptor family, member 2 | 86688-86974 |
| 272 | Rho-associated coiled-coil containing protein kinase 1 | 94052-94292 |
| 301 | MAP/microtubule affinity-regulating kinase 1 | 102310-102609 |
| 345 | glycogen synthase kinase 3 beta | 114424-114743 |
| 349 | adrenergic receptor kinase, beta 1 | 115587-115982 |
| 378 | tousled-like kinase 1 | 124295-124551 |
| 416 | PCTAIRE-motif protein kinase 1 | 134792-135023 |
| 420 | MAP/microtubule affinity-regulating kinase 2 | 135926-136274 |
| 432 | cyclin D1 | 139242-139629 |
| 434 | mitogen-activated protein kinase kinase kinase 7 | 139905-140195 |
| 448 | casein kinase 1, delta | 144005-144272 |
| 454 | PFTAIRE protein kinase 1 | 145534-145792 |
| 455 | PRP4 pre-mRNA processing factor 4 homolog B (yeast) | 145793-146023 |
| 459 | serine/threonine kinase 39, STE20/SPS1 | 146854-147131 |
| | homolog (yeast) | |
| 490 | Fyn proto-oncogene | 155354-155611 |
| 510 | calcium/calmodulin-dependent protein kinase II γ | 161048-161267 |
| 543 | Janus kinase 2 | 170408-170768 |
| 559 | carbamoyl-phosphate synthetase 2, aspartate | 174646-174897 |
| | transcarbamylase, and dihydroorotase | |
| 600 | casein kinase 1, gamma 1 | 186716-187114 |
| 634 | leucine-rich repeat kinase 1 | 197327-197719 |
| 644 | mitogen-activated protein kinase 6 | 200294-200550 |
| 662 | calcium/calmodulin-dependent protein kinase II, δ | 205498-205717 |
| 681 | MAP/microtubule affinity-regulating kinase 3 | 211317-211594 |
| 689 | budding uninhibited by benzimidazoles 1 homolog | 213750-213996 |
| | (S. cerevisiae) | |
| 725 | LIM motif-containing protein kinase 2 | 224252-224614 |
| 729 | homeodomain interacting protein kinase 3 | 225660-225908 |
| 730 | microtubule associated serine/threonine kinase 2 | 225909-226275 |
| 732 | transforming growth factor, beta receptor I | 226652-227037 |
| 829 | protein kinase, cAMP dependent, catalytic, beta | 256726-256960 |
| 836 | mitogen-activated protein kinase kinase kinase 12 | 258825-259201 |
| 864 | intestinal cell kinase | 267348-267605 |
| 870 | mitogen-activated protein kinase kinase kinase 3 | 269115-269501 |
| 871 | nemo like kinase | 269502-269739 |
| 873 | cyclin G associated kinase | 270072-270372 |
| 878 | mitogen-activated protein kinase 3 | 271504-271774 |
| 907 | G protein-coupled receptor kinase 6 | 281096-281476 |
| 929 | Rous sarcoma oncogene | 288625-288989 |
| 969 | thymoma viral proto-oncogene 2 | 301570-301889 |
| 1006 | large tumor suppressor 2 | 314156-314545 |
| 1049 | casein kinase 1, gamma 3 | 328602-328958 |
| 1057 | serine/threonine kinase 38 | 331497-331885 |
| 1074 | MAP kinase-activated protein kinase 2 | 336742-337085 |
| 1082 | tousled-like kinase 2 (Arabidopsis) | 339541-339778 |
| 1083 | serine/threonine kinase 40 | 339779-340105 |
| 1094 | SCY1-like 1 (S. cerevisiae) | 343589-343905 |
| 1098 | PCTAIRE-motif protein kinase 2 | 344918-345284 |
| 1105 | triple functional domain (PTPRF interacting) | 347214-347540 |
| 1158 | protein kinase N2 | 365489-365727 |
| 1173 | v-erb-b2 erythroblastic leukemia viral oncogene homolog | 370312-370704 |
| | 2, neuro/glioblastoma derived oncogene homolog (avian) | |
| 1188 | WEE 1 homolog 1 (S. pombe) | 375593-375982 |
| 1205 | mitogen-activated protein kinase-activated protein | 380855-381192 |
| | kinase 3 | |
| 1223 | conserved helix-loop-helix ubiquitous kinase | 386803-387186 |
| 1230 | mitogen-activated protein kinase 8 | 388975-389185 |
| 1245 | bone morphogenetic protein receptor, type 1A | 393916-394306 |
| 1248 | tripartite motif-containing 28 | 394982-395338 |
| 1283 | serine/arginine-rich protein specific kinase 1 | 406749-407114 |
| 1310 | mitogen-activated protein kinase kinase 4 | 415843-416086 |
| 1320 | platelet derived growth factor receptor, β polypeptide | 419363-419724 |
| 1360 | receptor-like tyrosine kinase | 433042-433431 |
| 1440 | TANK-binding kinase 1 | 460287-460685 |
| 1452 | DNA segment, Chr 8, ERATO Doi 82, expressed | 464366-464673 |
| 1472 | v-raf murine sarcoma 3611 viral oncogene homolog | 471108-471446 |
| 1496 | CDC-like kinase 3 | 479192-479450 |
| 1498 | casein kinase 1, epsilon | 479802-480166 |
| 1507 | serine/threonine kinase 24 (STE20 homolog, yeast) | 482939-483243 |
| 1534 | protein kinase D1 | 491875-492210 |
| 1615 | interleukin-1 receptor-associated kinase 2 | 519606-519900 |
| 1623 | v-raf-leukemia viral oncogene 1 | 522454-522805 |
| 1638 | polo-like kinase 2 (Drosophila) | 527681-527996 |
| 1640 | p21 protein (Cdc42/Rac)-activated kinase 2 | 528351-528713 |
| 1688 | serine/threonine kinase 16 | 544970-545325 |
| 1696 | ribosomal protein S6 kinase polypeptide 1 | 547863-548141 |
| 1700 | transforming growth factor, beta receptor II | 549106-549395 |
| 1719 | ataxia telangiectasia and Rad3 related | 555695-555945 |
| 1791 | insulin-like growth factor I receptor | 580583-580928 |
| 1793 | thymoma viral proto-oncogene 1 | 581286-581643 |
| 1798 | eukaryotic translation initiation factor 2 kinase 4 | 582987-583297 |
| 1802 | cyclin-dependent kinase 8 | 584337-584730 |
| 1821 | ribosomal protein S6 kinase, polypeptide 4 | 590773-591132 |
| 1822 | polo-like kinase 1 (Drosophila) | 591133-591528 |
| 1838 | proviral integration site 3 | 596508-596892 |
| 1839 | WNK lysine deficient protein kinase 1 | 596893-597187 |
| 1842 | MAP kinase-interacting serine/threonine kinase 2 | 597880-598207 |
| 1849 | NIMA-related expressed kinase 6 | 600327-600624 |
| 1853 | BMP2 inducible kinase | 601662-602044 |
| 1873 | protein kinase C, delta | 608454-608757 |
| 1874 | NIMA-related expressed kinase 9 | 608758-609143 |
| 1885 | interleukin-1 receptor-associated kinase 1 | 612534-612817 |
| 1953 | CDC42 binding protein kinase beta | 635482-635834 |
| 1956 | mitogen-activated protein kinase kinase 3 | 636446-636831 |
| 1967 | serum/glucocorticoid regulated kinase 1 | 640401-640729 |
| 1982 | mitogen-activated protein kinase kinase kinase 4 | 645415-645811 |
| 1985 | serine/threonine kinase 4 | 646540-646922 |
| 2022 | p21 protein (Cdc42/Rac)-activated kinase 1 | 658646-658945 |
| 2040 | STE20-like kinase (yeast) | 664580-664973 |
| 2058 | PX domain containing serine/threonine kinase | 670668-671043 |
| 2064 | TAO kinase 3 | 672877-673175 |
| 2074 | SH3-binding kinase 1 | 676411-676808 |
| 2089 | nuclear receptor binding protein 1 | 681455-681766 |
| 2094 | polo-like kinase 3 (Drosophila) | 683175-683550 |
| 2096 | mitogen-activated protein kinase 14 | 683848-684174 |
| 2157 | macrophage stimulating 1 receptor (c-met-related | 704139-704461 |
| | tyrosine kinase) | |
| 2224 | protein kinase N1 | 726766-727146 |
| 2252 | mitogen-activated protein kinase kinase kinase 5 | 736639-737018 |
| 2281 | casein kinase 1, alpha 1 | 746332-746692 |
| 2313 | testis specific protein kinase 1 | 757254-757624 |
| 2321 | U2AF homology motif (UHM) kinase 1 | 759990-760335 |
| 2348 | casein kinase 1, gamma 2 | 769048-769436 |
| 2371 | activin A receptor, type 1 | 776681-777035 |
| 2391 | TYRO3 protein tyrosine kinase 3 | 783438-783823 |
| 2395 | platelet derived growth factor receptor, polypeptide | 784759-785127 |
| 2429 | SNF related kinase | 796332-796725 |
| 2433 | met proto-oncogene | 797652-798038 |
| 2434 | mitogen-activated protein kinase kinase 1 | 798039-798333 |
| 2450 | receptor (TNFRSF)-interacting serine-threonine kinase 1 | 803414-803712 |
| 2453 | cell division cycle 2-like 5 (cholinesterase-related | 804372-804761 |
| | cell division controller) | |
| 2498 | SCY1-like 2 (S. cerevisiae) | 819902-820288 |
| 2500 | Eph receptor A2 | 820644-820974 |
| 2530 | misshapen-like kinase 1 (zebrafish) | 830880-831232 |
| 2567 | Unc-51 like kinase 1 (C. elegans) | 843486-843843 |
| 2569 | cyclin-dependent kinase 7 (homolog of Xenopus | 844194-844512 |
| | MO15 cdk-activating kinase) | |
| 2605 | protein serine kinase H1 | 856267-856572 |
| 2606 | NIMA-related expressed kinase 7 | 856573-856901 |
| 2609 | Janus kinase 1 | 857488-857805 |
| 2615 | c-mer proto-oncogene tyrosine kinase | 859390-859712 |
| 2649 | serine/threonine kinase 25 (yeast) | 870722-871034 |
| 2656 | maternal embryonic leucine zipper kinase | 873142-873499 |
| 2660 | transforming growth factor beta regulated gene 4 | 874486-874847 |
| 2678 | mitogen-activated protein kinase kinase kinase 6 | 880782-881178 |
| 2685 | c-src tyrosine kinase | 883213-883509 |
| 2690 | protein kinase, cAMP dependent, catalytic, alpha | 884918-885283 |
| 2697 | RIKEN cDNA C230081A13 gene | 887214-887504 |
| 2727 | mitogen-activated protein kinase 1 | 897474-897851 |
| 2728 | STE20-related kinase adaptor alpha | 897852-898184 |
| 2739 | LIM-domain containing, protein kinase | 901587-901936 |
| 2767 | mitogen-activated protein kinase kinase kinase 10 | 911247-911607 |
| 2797 | mitogen-activated protein kinase 10 | 921494-921818 |
| 2815 | serine/threonine kinase 3 (Ste20, yeast homolog) | 927749-928072 |
| 2821 | protein kinase N3 | 929703-929953 |
| 2844 | large tumor suppressor | 937654-937969 |
| 2854 | leucine-rich repeat kinase 2 | 940941-941325 |
| 2917 | phosphatidylinositol 3 kinase, regulatory subunit, | 962493-962788 |
| | polypeptide 4, p150 | |
| 2965 | protein kinase, DNA activated, catalytic polypeptide | 979242-979576 |
| 2966 | doublecortin-like kinase 1 | 979577-979919 |
| 3005 | activin receptor IIA | 993008-993293 |
| 3016 | Unc-51 like kinase 2 (C. elegans) | 996609-996900 |
| 3028 | branched chain ketoacid dehydrogenase kinase | 1000498-1000839 |
| 3066 | mitogen-activated protein kinase 3 | 1013377-1013718 |
| 3072 | p21 protein (Cdc42/Rac)-activated kinase 4 | 1015266-1015566 |
| 3110 | protein kinase, membrane associated | 1028441-1028755 |
| | tyrosine/threonine 1 | |
| 3137 | eukaryotic translation initiation factor 2 kinase 1 | 1037660-1038052 |
| 3141 | PAS domain containing serine/threonine kinase | 1039167-1039558 |
| 3145 | cyclin D3 | 1040554-1040910 |
| 3170 | PTK2 protein tyrosine kinase 2 | 1049366-1049709 |
| 3215 | c-abl oncogene 1, receptor tyrosine kinase | 1064790-1065134 |
| 3234 | FAST kinase domains 5 | 1071097-1071485 |
| 3264 | ribosomal protein S6 kinase polypeptide 3 | 1081273-1081650 |
| 3293 | glycogen synthase kinase 3 alpha | 1091000-1091318 |
| 3302 | integrin linked kinase | 1094162-1094466 |
| 3325 | fer (fms/fps related) protein kinase, testis specific 2 | 1101741-1102066 |
| 3390 | cell division cycle 2-like 1 | 1124002-1124331 |
| 3497 | CDC-like kinase 2 | 1159741-1160065 |
| 3517 | aarF domain containing kinase 1 | 1166401-1166741 |
| 3551 | RIKEN cDNA B230120H23 gene | 1177903-1178190 |
| 3583 | checkpoint kinase 1 homolog (S. pombe) | 1188354-1188736 |
| 3598 | cyclin-dependent kinase 2 | 1193336-1193684 |
| 3636 | vaccinia related kinase 3 | 1206468-1206770 |
| 3672 | MAP kinase-activated protein kinase 5 | 1218590-1218943 |
| 3697 | tyrosine kinase, non-receptor, 2 | 1227011-1227293 |
| 3752 | calcium/calmodulin-dependent protein kinase 2, β | 1245765-1246095 |
| 3761 | ataxia telangiectasia mutated homolog (human) | 1248864-1249255 |
| 3792 | salt inducible kinase 1 | 1259549-1259840 |
| 3803 | phosphoinositide-3-kinase, class 3 | 1263190-1263540 |
| 3810 | aarF domain containing kinase 2 | 1265631-1265906 |
| 3818 | tripartite motif-containing 24 | 1268181-1268568 |
| 3839 | MAP kinase-interacting serine/threonine kinase 1 | 1275270-1275564 |
| 3946 | polo-like kinase 4 (Drosophila) | 1310666-1311034 |
| 4001 | mitogen-activated protein kinase kinase 2 | 1329109-1329497 |
| 4017 | Janus kinase 3 | 1334368-1334721 |
| 4043 | CDC like kinase 4 | 1343146-1343482 |
| 4045 | SCY1-like 3 (S. cerevisiae) | 1343876-1344245 |
| 4071 | NIMA-related expressed kinase 2 | 1352509-1352861 |
| 4151 | vaccinia related kinase 2 | 1379213-1379553 |
| 4171 | casein kinase 2, alpha prime polypeptide | 1385888-1386249 |
| 4193 | mitogen-activated protein kinase 1 | 1393467-1393856 |
| 4201 | eukaryotic translation initiation factor 2 kinase 3 | 1396283-1396617 |
| 4255 | budding uninhibited by benzimidazoles 1 homolog, | 1414236-1414628 |
| | beta (S. cerevisiae) | |
| 4264 | vaccinia related kinase 1 | 1417312-1417688 |
| 4268 | STE20-related kinase adaptor beta | 1418669-1418996 |
| 4275 | FAST kinase domains 2 | 1421149-1421474 |
| 4299 | cyclin-dependent kinase 9 (CDC2-related kinase) | 1429472-1429796 |
| 4365 | lemur tyrosine kinase 2 | 1451144-1451458 |
| 4404 | Yamaguchi sarcoma viral (v-yes) oncogene homolog 1 | 1464339-1464640 |
| 4414 | cyclin-dependent kinase 5 | 1467595-1467925 |
| 4488 | bone morphogenic protein receptor, type II | 1492190-1492490 |
| | (serine/threonine kinase) | |
| 4502 | testis-specific kinase 2 | 1496336-1496660 |
| 4632 | cell division cycle 7 (S. cerevisiae) | 1539427-1539781 |
| 4652 | mitogen-activated protein kinase kinase 5 | 1545970-1546310 |
| 4686 | mitogen-activated protein kinase 1 | 1557428-1557817 |
| 4715 | ribonuclease L (2′,5′-oligoisoadenylate | 1567391-1567708 |
| | synthetase-dependent) | |
| 4744 | fibroblast growth factor receptor 1 | 1577052-1577365 |
| 4770 | protein kinase D3 | 1585680-1585976 |
| 4839 | cyclin-dependent kinase 4 | 1609522-1609852 |
| 4856 | protein kinase C, iota | 1615321-1615627 |
| 4867 | ribosomal protein S6 kinase, polypeptide 2 | 1618874-1619239 |
| 4903 | tyrosine kinase 2 | 1631375-1631670 |
| 4904 | FAST kinase domains 3 | 1631671-1632058 |
| 4928 | Phosphorylase kinase, gamma 2 (testis) | 1639845-1640227 |
| 4947 | protein kinase, AMP-activated, β 1 non-catalytic subunit | 1646526-1646858 |
| 4952 | tribbles homolog 3 (Drosophila) | 1648199-1648515 |
| 4980 | natriuretic peptide receptor 2 | 1658017-1658362 |
| 5012 | NIMA-related expressed kinase 8 | 1668806-1669200 |
| 5119 | protein kinase, X-linked | 1705097-1705372 |
| 5127 | interleukin-1 receptor-associated kinase 4 | 1707814-1708142 |
| 5155 | protein kinase, AMP-activated, γ 1 non-catalytic subunit | 1717347-1717743 |
| 5205 | serine/threonine kinase 10 | 1734723-1735086 |
| 5258 | protein kinase C, eta | 1752699-1753060 |
| 5260 | receptor (TNFRSF)-interacting serine-threonine kinase 2 | 1753377-1753673 |
| 5303 | protein kinase, AMP-activated, γ 2 non-catalytic subunit | 1767887-1768173 |
| 5443 | CHK2 checkpoint homolog (S. pombe) | 1817364-1817648 |
| 5466 | dual-specificity tyrosine-(Y)-phosphorylation | 1825671-1825984 |
| | regulated kinase 3 | |
| 5513 | NIMA-related expressed kinase 1 | 1842362-1842733 |
| 5526 | PDZ binding kinase | 1846866-1847240 |
| 5543 | Ttk protein kinase | 1852758-1853100 |
| 5580 | cell division cycle 2 homolog A (S. pombe) | 1865374-1865693 |
| 5636 | mitogen-activated protein kinase 7 | 1885325-1885696 |
| 5698 | aurora kinase A | 1907469-1907831 |
| 5753 | Eph receptor B3 | 1927508-1927885 |
| 5812 | oxidative-stress responsive 1 | 1948788-1949181 |
| 5833 | cyclin H | 1956302-1956671 |
| 5892 | inhibitor of kappaB kinase epsilon | 1978013-1978395 |
| 5902 | cell cycle related kinase | 1981792-1982170 |
| 5944 | serine/threonine kinase 38 like | 1997111-1997478 |
| 5974 | tribbles homolog 1 (Drosophila) | 2008081-2008383 |
| 6029 | mixed lineage kinase domain-like | 2027899-2028286 |
| 6121 | discoidin domain receptor family, member 1 | 2061270-2061663 |
| 6141 | aurora kinase B | 2068620-2068994 |
| 6178 | mitogen-activated protein kinase kinase kinase 14 | 2081730-2082108 |
| 6215 | RIKEN cDNA E130304F04 gene | 2095357-2095740 |
| 6281 | cyclin-dependent kinase-like 2 (CDC2-related kinase) | 2118747-2119146 |
| 6305 | dual-specificity tyrosine-(Y)-phosphorylation | 2127434-2127800 |
| | regulated kinase 2 | |
| 6404 | cyclin-dependent kinase (CDC2-like) 10 | 2162918-2163302 |
| 6480 | cyclin-dependent kinase 6 | 2189891-2190242 |
| 6633 | protein kinase D2 | 2243758-2244155 |
| 6653 | WNK lysine deficient protein kinase 4 | 2250760-2251118 |
| 6731 | G protein-coupled receptor kinase 5 | 2278131-2278499 |
| 6882 | aurora kinase C | 2329723-2330035 |
| 6891 | cyclin-dependent kinase-like 1 (CDC2-related kinase) | 2332108-2332434 |
| 6929 | RIKEN cDNA 4930444A02 gene | 2344573-2344930 |
| 6980 | p21 protein (Cdc42/Rac)-activated kinase 3 | 2361621-2361941 |
| 7029 | ribosomal protein S6 kinase, polypeptide 5 | 2378152-2378437 |
| 7063 | CDC-like kinase 1 | 2389819-2390124 |
| 7073 | PDLIM1 interacting kinase 1 like | 2393123-2393501 |
| 7086 | salt inducible kinase 2 | 2397231-2397606 |
| 7124 | homeodomain interacting protein kinase 2 | 2409808-2410107 |
| 7144 | serum/glucocorticoid regulated kinase 3 | 2416403-2416787 |
| 7151 | germ cell-specific gene 2 | 2418878-2419222 |
| 7165 | cyclin-dependent kinase-like 3 | 2423119-2423482 |
| 7167 | fibroblast growth factor receptor 3 | 2423777-2424112 |
| 7224 | NIMA-related expressed kinase 4 | 2443004-2443301 |
| 7242 | hormonally upregulated Neu-associated kinase | 2449048-2449437 |
| 7289 | inhibitor of kappaB kinase beta | 2464074-2464378 |
| 7487 | serum/glucocorticoid regulated kinase 2 | 2529508-2529774 |
| 7501 | 3-phosphoinositide dependent protein kinase-1 | 2534260-2534622 |
| 7507 | lymphocyte protein tyrosine kinase | 2536052-2536408 |
| 7604 | microtubule associated serine/threonine kinase-like | 2567713-2568021 |
| 7630 | serine/threonine kinase 11 | 2575716-2576017 |
| 7661 | MAP/microtubule affinity-regulating kinase 4 | 2585629-2585955 |
| 7781 | proviral integration site 1 | 2626615-2627001 |
| 7784 | serine/threonine kinase 17b (apoptosis-inducing) | 2627742-2628087 |
| 7797 | protein kinase C, epsilon | 2632117-2632509 |
| 7808 | myosin, light polypeptide kinase 2, skeletal muscle | 2635957-2636283 |
| 7841 | NIMA-related expressed kinase 3 | 2646895-2647246 |
| 7917 | PTK2 protein tyrosine kinase 2 beta | 2672668-2672997 |
| 7980 | endothelial-specific receptor tyrosine kinase | 2693563-2693919 |
| 8109 | thymoma viral proto-oncogene 3 | 2735270-2735575 |
| 8123 | citron | 2740025-2740319 |
| 8173 | NUAK family, SNF1-like kinase, 1 | 2755489-2755818 |
| 8206 | activin A receptor, type 1B | 2766172-2766565 |
| 8328 | FAST kinase domains 1 | 2806153-2806512 |
| 8469 | activin receptor IIB | 2854148-2854509 |
| 8556 | serine/threonine kinase 30 | 2882719-2883094 |
| 8662 | death-associated protein kinase 3 | 2918007-2918383 |
| 8760 | testis-specific serine kinase 6 | 2949013-2949363 |
| 8792 | RIKEN cDNA A630047E20 gene | 2959129-2959498 |
| 8890 | testis-specific serine kinase 4 | 2988076-2988379 |
| 8946 | G protein-coupled receptor kinase 1 | 3003705-3003945 |
| 9035 | PAN3 polyA specific ribonuclease subunit homolog | 3027117-3027350 |
| | (S. cerevisiae) | |
| 9149 | mitogen-activated protein kinase 2 | 3055949-3056195 |
| 9202 | calcium/calmodulin-dependent protein kinase IV | 3067906-3067965 |
| 9218 | ribosomal protein S6 kinase polypeptide 1 | 3070827-3071085 |
| 9232 | apoptosis-associated tyrosine kinase | 3074031-3074270 |
| 9252 | Eph receptor B4 | 3078422-3078630 |
| 9266 | serine/threonine/tyrosine kinase 1 | 3081287-3081520 |
| 9338 | testis-specific serine kinase 1 | 3097427-3097661 |
| 9460 | G protein-coupled receptor kinase 4 | 3120208-3120400 |
| 9526 | NUAK family, SNF1-like kinase, 2 | 3130443-3130616 |
| 9577 | FMS-like tyrosine kinase 1 | 3137414-3137564 |
| 9643 | testis-specific serine kinase 5 | 3143809-3143951 |
| 9672 | calcium/calmodulin-dependent protein kinase 1, | 3146563-3146684 |
| 9688 | tyrosine kinase, non-receptor, 1 | 3147699-3147819 |
| 9721 | phosphorylase kinase gamma 1 | 3149851-3149854 |
| 9722 | mitogen-activated protein kinase 7 | 3149855-3149946 |
| 3157213 | mitogen-activated protein kinase 5 | 3233617-3233716 |
| 3157247 | endoplasmic reticulum (ER) to nucleus signaling 1 | 3179284-3179383 |
| 3157267 | mitogen-activated protein kinase kinase kinase 2 | 3185971-3186070 |
| 3157347 | fibroblast growth factor receptor 4 | 3276349-3276448 |
| 3157427 | dual serine/threonine and tyrosine protein kinase | 3163684-3163783 |
| 3157453 | testis expressed gene 14 | 3276149-3276248 |
| 3157487 | NIMA-related expressed kinase 11 | 3167184-3167283 |
| 3157527 | NIMA-related expressed kinase 5 | 3275849-3275948 |
| 3157545 | death-associated protein kinase 2 | 3254417-3254516 |
| 3157639 | spleen tyrosine kinase | 3259705-3259804 |
| 3157684 | doublecortin-like kinase 2 | 3170684-3170783 |
| 3157692 | myosin, light polypeptide kinase | 3220991-3221090 |
| 3157728 | NA | 3235317-3235416 |
| 3157785 | TRAF2 and NCK interacting kinase | 3264805-3264904 |
| 3157794 | tribbles homolog 2 (Drosophila) | 3204997-3205096 |
| 3157808 | unc-51-like kinase 3 (C. elegans) | 3229117-3229216 |
| 3157827 | insulin receptor | 3239817-3239916 |
| 3157880 | PTK7 protein tyrosine kinase 7 | 3277549-3277648 |
| 3157993 | epidermal growth factor receptor | 3166784-3166883 |
| 3158134 | anaplastic lymphoma kinase | 3247317-3247416 |
| 3158136 | receptor tyrosine kinase-like orphan receptor 1 | 3228817-3228916 |
| 3158179 | NA | 3252117-3252216 |
| 3158184 | calcium/calmodulin-dependent protein kinase II | 3257905-3258004 |
| 3158194 | NA | 3255705-3255804 |
| 3158209 | fibroblast growth factor receptor 2 | 3207458-3207557 |
| 3158279 | unc-51-like kinase 4 (C. elegans) | 3273396-3273495 |
| 3158375 | megakaryocyte-associated tyrosine kinase | 3204897-3204996 |
| 3158394 | bone morphogenetic protein receptor, type 1B | 3218291-3218390 |

TABLE 61
Cytochrome reductases

| SEQ | | Avg | siRNA SEQ |
|---|---|---|---|
| ID NO: | Description | Cov | ID NOs: |
| 1124 | P450 (cytochrome) oxidoreductase | 18.96 | 353642-353994 |
| 1759 | cytochrome b5 reductase 4 | 14.829 | 569460-569777 |
| 2330 | ubiquinol-cytochrome c reductase complex | 18.852 | 763043-763396 |
| | chaperone, CBP3 homolog (yeast) | | |
| 3795 | ubiquinol-cytochrome c reductase core protein 1 | 109.161 | 1260523-1260890 |
| 3799 | cytochrome b5 reductase 3 | 78.623 | 1261910-1262218 |
| 3897 | cytochrome b reductase 1 | 1.445 | 1294703-1295101 |
| 4548 | ubiquinol cytochrome c reductase core protein 2 | 74.045 | 1511637-1511998 |
| 5706 | ubiquinol-cytochrome c reductase, Rieske iron- | 78.928 | 1910358-1910701 |
| | sulfur polypeptide 1 | | |
| 6495 | cytochrome b5 reductase 1 | 7.465 | 2195311-2195681 |
| 8631 | ubiquinol-cytochrome c reductase hinge protein | 4.546 | 2907991-2908330 |
| 8675 | ubiquinol-cytochrome c reductase binding protein | 3.239 | 2922032-2922391 |
| 9127 | ubiquinol-cytochrome c reductase, complex III | 2.023 | 3050777-3051054 |
| | subunit VII | | |

TABLE 21
Ubiquitin-thiolesterases

| SEQ | | | Avg | siRNA SEQ |
|---|---|---|---|---|
| ID NO: | consL | Description | Cov | ID NOs: |
| | 7293 | ubiquitin specific peptidase 9, X chromosome | 6.127 | 9772-10147 |
| 93 | 3839 | ubiquitin specific peptidase 48 | 5.077 | 39432-39822 |
| 95 | 3832 | ubiquitin specific peptidase 7 | 16.622 | 40175-40559 |
| 103 | 3754 | ubiquitin specific peptidase 40 | 2.183 | 42743-43018 |
| 273 | 3151 | ubiquitin specific peptidase 47 | 11.289 | 94293-94582 |
| 276 | 3145 | cylindromatosis (turban tumor syndrome) | 5.717 | 95119-95374 |
| 335 | 3057 | ubiquitin specific peptidase 8 | 10.752 | 111384-111738 |
| 514 | 2833 | ubiquitin specific peptidase 10 | 11.689 | 161987-162319 |
| 598 | 2741 | ubiquitin specific peptidase 15 | 10.777 | 185975-186368 |
| 625 | 2714 | ubiquitin specific peptidase 25 | 1.533 | 194182-194422 |
| 834 | 2567 | ubiquitin specific peptidase 38 | 3.655 | 258184-258571 |
| 931 | 2523 | ubiquitin specific peptidase 4 | 13.735 | 289262-289658 |
| | | (proto-oncogene) | | |
| 965 | 2501 | ubiquitin specific peptidase 16 | 11.237 | 300334-300663 |
| 980 | 2494 | ubiquitin specific peptidase 28 | 6.027 | 305222-305581 |
| 1311 | 2331 | ubiquitin specific peptidase 12 | 3.674 | 416087-416477 |
| 1499 | 2245 | ubiquitin specific peptidase 33 | 3.642 | 480167-480565 |
| 1502 | 2244 | ubiquitin specific peptidase 19 | 7.049 | 481244-481580 |
| 1541 | 2233 | ubiquitin specific peptidase 1 | 1.24 | 494093-494468 |
| 1612 | 2205 | OTU domain containing 7B | 0.437 | 518572-518901 |
| 1660 | 2188 | ubiquitin specific peptidase 54 | 0.655 | 535568-535921 |
| 1941 | 2098 | ubiquitin specific peptidase 11 | 3.914 | 631257-631579 |
| 2267 | 1990 | ubiquitin specific peptidase 14 | 2.01 | 741691-741995 |
| 2275 | 1989 | ubiquitin specific peptidase 39 | 11.625 | 744331-744665 |
| 2303 | 1982 | ubiquitin specific peptidase 46 | 1.193 | 753953-754261 |
| 2460 | 1942 | Brca1 associated protein 1 | 3.462 | 806747-807089 |
| 2596 | 1909 | ubiquitin specific peptidase 21 | 10.965 | 853543-853866 |
| 2634 | 1899 | ubiquitin specific peptidase 22 | 1.692 | 865729-866104 |
| 3030 | 1785 | ubiquitin specific peptidase 5 (isopeptidase T) | 13.894 | 1001194-1001562 |
| 3074 | 1774 | BRCA1/BRCA2-containing complex, subunit 3 | 1.488 | 1015902-1016231 |
| 3536 | 1662 | ubiquitin specific peptidase 27, | 0.685 | 1172962-1173239 |
| | | X chromosome | | |
| 3558 | 1654 | ubiquitin specific peptidase 52 | 3.654 | 1180058-1180445 |
| 3714 | 1620 | ubiquitin specific peptidase 30 | 0.966 | 1232956-1233353 |
| 3842 | 1586 | myb-like, SWIRM and MPN domains 1 | 0.676 | 1276194-1276510 |
| 3915 | 1570 | ubiquitin specific peptidase 3 | 6.65 | 1300512-1300831 |
| 4057 | 1535 | ubiquitin specific peptidase 18 | 3.571 | 1347935-1348245 |
| 4072 | 1530 | proteasome (prosome, macropain) 26S | 67.811 | 1352862-1353184 |
| | | subunit, non-ATPase, 14 | | |
| 4107 | 1522 | ubiquitin carboxyl-terminal esterase L5 | 10.895 | 1364288-1364643 |
| 4509 | 1434 | ubiquitin specific peptidase 20 | 0.904 | 1498598-1498950 |
| 4875 | 1353 | OTU domain containing 5 | 3.986 | 1621572-1621944 |
| 5615 | 1187 | ubiquitin specific peptidase like 1 | 1.464 | 1877785-1878169 |
| 5649 | 1178 | STAM binding protein | 2.283 | 1889758-1890088 |
| 6996 | 881 | ubiquitin carboxyl-terminal esterase L3 | 2.405 | 2367046-2367358 |
| | | (ubiquitin thiolesterase) | | |
| 8860 | 427 | ubiquitin carboxyl-terminal esterase L4 | 0.446 | 2979143-2979234 |
| 8992 | 395 | ataxin 3 | 0.087 | 3016154-3016402 |
| 9384 | 291 | ubiquitin specific peptidase 53 | 0.073 | 3106251-3106450 |
| 3157441 | 263 | ubiquitin specific peptidase 50 | 0.152 | 3267405-3267504 |
| 3157521 | 192 | ubiquitin specific peptidase 37 | 0.027 | 3170784-3170883 |
| 3157574 | 1203 | ubiquitin specific petidase 45 | 0.416 | 3242017-3242116 |

TABLE 23
E3 Ubiquitin Protein ligases

| SEQ | | | Avg | siRNA SEQ |
|---|---|---|---|---|
| ID NO: | consL | Description | Cov | ID NOs: |
| 9 | 4809 | ubiquitin protein ligase E3 component n-recognin 3 | 2.236 | 12279-12498 |
| 48 | 4159 | SMAD specific E3 ubiquitin protein ligase 2 | 7.495 | 24792-25162 |
| 64 | 3999 | itchy, E3 ubiquitin protein ligase | 4.833 | 29919-30278 |
| 101 | 3757 | ubiquitin protein ligase E3 component n-recognin 5 | 9.431 | 42166-42421 |
| 143 | 3560 | ubiquitin protein ligase E3C | 11.999 | 55140-55421 |
| 936 | 2521 | ubiquitin protein ligase E3 component n-recognin 2 | 3.175 | 290987-291365 |
| 1355 | 2311 | HECT domain and ankyrin repeat containing, | 11.689 | 431371-431703 |
| | | E3 ubiquitin protein ligase 1 | | |
| 2414 | 1956 | SMAD specific E3 ubiquitin protein ligase 1 | 0.804 | 791272-791663 |
| 3279 | 1724 | ubiquitin protein ligase E3 component n- | 2.904 | 1086176-1086492 |
| | | recognin 7 (putative) | | |
| 3531 | 1663 | ubiquitin protein ligase E3B | 1.82 | 1171311-1171631 |
| 3906 | 1573 | WW domain containing E3 ubiquitin protein ligase 2 | 1.581 | 1297605-1297894 |
| 4078 | 1528 | WW domain containing E3 ubiquitin protein ligase 1 | 0.308 | 1354729-1355093 |
| 6165 | 1066 | G2/M-phase specific E3 ubiquitin ligase | 0.358 | 2077605-2078002 |
| 6645 | 960 | ubiquitin protein ligase E3 component n-recognin 1 | 0.266 | 2248043-2248415 |
| 6760 | 934 | ubiquitin protein ligase E3A | 0.576 | 2287890-2288245 |
| 3157485 | 2014 | ubiquitin protein ligase E3 component n-recognin 4 | 0.639 | 3209658-3209757 |
| 3157673 | 192 | HECT, C2 and WW domain containing E3 | 0.017 | 3269496-3269595 |
| | | ubiquitin protein ligase 2 | | |

TABLE 24
STATs

| SEQ | | | Avg | siRNA SEQ |
|---|---|---|---|---|
| ID NO: | consL | Description | Cov | ID NOs: |
| 540 | 2799 | signal transducer & activator of | 1.323 | 169415-169753 |
| | | transcription 5B | | |
| 887 | 2543 | signal transducer & activator of transcription 1 | 5.548 | 274540-274924 |
| 2234 | 2001 | signal transducer & activator of transcription 6 | 2.945 | 730267-730586 |
| 2249 | 1997 | signal transducer & activator of transcription 3 | 0.987 | 735545-735924 |
| 3913 | 1571 | signal transducer & activator of | 1.268 | 1299843-1300222 |
| | | transcription 5A | | |
| 3157484 | 433 | signal transducer & activator of transcription 2 | 0.099 | 3168284-3168383 |
| 3157597 | 252 | signal transducer & activator of transcription 4 | 0.087 | 3226517-3226616 |

TABLE 27
Stress Response Genes

| SEQ | | | Avg | siRNA SEQ |
|---|---|---|---|---|
| ID NO: | consL | Description | Cov | ID NOs: |
| 74 | 3956 | mitogen-activated protein kinase kinase | 10.121 | 33333-33668 |
| | | kinase kinase 4 | | |
| 221 | 3285 | hypoxia up-regulated 1 | 59.506 | 78625-79007 |
| 279 | 3139 | methyl CpG binding protein 2 | 1.23 | 95910-96141 |
| 345 | 3034 | glycogen synthase kinase 3 beta | 0.647 | 114424-114743 |
| 444 | 2900 | heat shock protein 4 | 15.95 | 142820-143094 |
| 476 | 2865 | heat shock 105 kDa/110 kDa protein 1 | 19.863 | 151195-151420 |
| 485 | 2858 | AHA1, activator of heat shock protein | 16.103 | 153831-154084 |
| | | ATPase homolog 2 (yeast) | | |
| 579 | 2758 | heat shock protein 90, β(Grp94), member 1 | 606.207 | 180574-180954 |
| 594 | 2744 | heat shock protein 90, (cytosolic), class A | 93.844 | 184698-184927 |
| | | member 1 | | |
| 827 | 2572 | heat shock protein 9 | 28.56 | 255926-256325 |
| 977 | 2496 | heat shock protein 90 alpha (cytosolic), class | 609.5 | 304274-304591 |
| | | B member 1 | | |
| 1384 | 2293 | TNF receptor-associated protein 1 | 66.2 | 441242-441639 |
| 1489 | 2250 | mitogen-activated protein kinase associated | 9.725 | 476915-477307 |
| | | protein 1 | | |
| 1798 | 2143 | eukaryotic translation initiation factor 2 alpha | 2.779 | 582987-583297 |
| | | kinase 4 | | |
| 1842 | 2130 | MAP kinase-interacting serine/threonine | 2.895 | 597880-598207 |
| | | kinase 2 | | |
| 1967 | 2087 | serum/glucocorticoid regulated kinase 1 | 4.001 | 640401-640729 |
| 1979 | 2085 | histone deacetylase 5 | 7.779 | 644628-644970 |
| 2076 | 2052 | DnaJ (Hsp40) homolog, subfamily B, | 9.75 | 677203-677558 |
| | | member 1 | | |
| 2096 | 2045 | mitogen-activated protein kinase 14 | 7.294 | 683848-684174 |
| 2272 | 1990 | heat shock factor 2 | 2.598 | 743398-743788 |
| 2297 | 1984 | protein phosphatase 3, catalytic subunit, | 4.715 | 751950-752267 |
| | | alpha isoform | | |
| 2372 | 1964 | Ser (or Cys) peptidase inhibitor clade H member 1 | 125.59 | 777036-777317 |
| 2530 | 1925 | misshapen-like kinase 1 (zebrafish) | 1.615 | 830880-831232 |
| 2756 | 1869 | heat shock factor 1 | 25.227 | 907582-907889 |
| 2779 | 1862 | homocysteine-inducible, endoplasmic reticulum | 19.826 | 915394-915727 |
| | | stress-inducible, ubiquitin-like domain member 1 | | |
| 2929 | 1822 | protein disulfide isomerase associated 5 | 42.884 | 966735-967056 |
| 2974 | 1807 | heat shock protein 2 | 5.538 | 982428-982785 |
| 3063 | 1776 | heat shock protein 8 | 38.69 | 1012333-1012621 |
| 3137 | 1761 | eukaryotic translation initiation factor 2 alpha | 9.682 | 1037660-1038052 |
| | | kinase 1 | | |
| 3151 | 1757 | cancer susceptibility candidate 3 | 6.742 | 1042529-1042877 |
| 3589 | 1647 | calmodulin binding transcription activator 2 | 0.784 | 1190341-1190653 |
| 3699 | 1623 | transforming, acidic coiled-coil containing | 13.073 | 1227651-1228044 |
| | | protein 3 | | |
| 3754 | 1611 | isocitrate dehydrogenase 2 (NADP+), | 8.177 | 1246485-1246791 |
| | | mitochondrial | | |
| 3839 | 1586 | MAP kinase-interacting serine/threonine | 2.216 | 1275270-1275564 |
| | | kinase 1 | | |
| 3943 | 1563 | eukaryotic translation initiation factor 2, subnt 1 | 14.063 | 1309599-1309969 |
| 4201 | 1500 | eukaryotic translation initiation factor 2 | 2.46 | 1396283-1396617 |
| | | kinase 3 | | |
| 4434 | 1453 | protein kinase, interferon inducible double | 5.527 | 1474052-1474353 |
| | | stranded RNA dependent activator | | |
| 4947 | 1338 | protein kinase, AMP-activated, beta 1 non- | 5.753 | 1646526-1646858 |
| | | catalytic subunit | | |
| 5002 | 1323 | AHA1, activator of heat shock protein | 93.621 | 1665415-1665746 |
| | | ATPase homolog 1 (yeast) | | |
| 5155 | 1287 | protein kinase, AMP-activated, gamma 1 | 12.934 | 1717347-1717743 |
| | | non-catalytic subunit | | |
| 5251 | 1271 | antigenic determinant of rec-A protein | 1.928 | 1750245-1750559 |
| 5295 | 1259 | nuclear receptor subfamily 4, group A, | 0.73 | 1765734-1766070 |
| | | member 2 | | |
| 5303 | 1258 | protein kinase, AMP-activated, gamma 2 | 0.729 | 1767887-1768173 |
| | | non-catalytic subunit | | |
| 5406 | 1236 | cold inducible RNA binding protein | 32.931 | 1804466-1804836 |
| 5424 | 1231 | SMT3 suppressor of mif two 3 homolog 1 | 10.803 | 1810772-1811128 |
| | | (yeast) | | |
| 6622 | 965 | pyrroline-5-carboxylate reductase 1 | 0.9 | 2239835-2240228 |
| 7418 | 785 | myeloid differentiation primary response | 2.514 | 2506840-2507215 |
| | | gene 116 | | |
| 7981 | 638 | Parkinson disease (autosomal recessive, | 47.839 | 2693920-2694252 |
| | | early onset) 7 | | |
| 8048 | 615 | RIKEN cDNA 2310016C08 gene | 1.503 | 2715913-2716256 |
| 8085 | 605 | protein phosphatase 1, regulatory (inhibitor) | 0.176 | 2727942-2728269 |
| | | subunit 15b | | |
| 8155 | 587 | sphingomyelin phosphodiesterase 3, neutral | 0.179 | 2750331-2750645 |
| 8336 | 539 | heat shock protein 1 | 3.124 | 2809108-2809434 |
| 8405 | 517 | heat shock protein 1 (chaperonin 10) | 4.477 | 2833031-2833420 |
| 8780 | 444 | HIG1 domain family, member 1A | 0.685 | 2955263-2955620 |
| 8954 | 403 | junction-mediating and regulatory protein | 0.09 | 3005715-3006035 |
| 9679 | 173 | heat shock protein 1B | 0.091 | 3147029-3147080 |
| 9722 | 149 | mitogen-activated protein kinase kinase 7 | 0.089 | 3149855-3149946 |
| 3157247 | 594 | endoplasmic reticulum (ER) to nucleus | 0.18 | 3179284-3179383 |
| | | signaling 1 | | |
| 3157505 | 644 | crystallin, alpha B | 0.99 | 3280749-3280848 |
| 3157706 | 999 | family with sequence similarity 129, member A | 0.792 | 3219891-3219990 |
| 3158121 | 3735 | transformation related protein 53 inducible | 2.567 | 3197071-3197170 |
| | | nuclear protein 1 | | |
| 3158350 | 787 | response to stress | 0.417 | 3201697-3201796 |

TABLE 28
Glycosyltransferases

| SEQ | | | Avg | siRNA SEQ |
|---|---|---|---|---|
| ID NO: | consL | Description | Cov | ID NOs: |
| 150 | 3549 | UDP-N-acetyl-alpha-D-galactosamine:polypeptide | 11.757 | 57147-57422 |
| | | N-acetylgalactosaminyltransferase 1 | | |
| 178 | 3411 | UDP-N-acetyl-alpha-D-galactosamine:polypeptide | 22.835 | 65737-65999 |
| | | N-acetylgalactosaminyltransferase 2 | | |
| 270 | 3158 | UDP-GalNAc:betaGlcNAc beta 1,3- | 4.224 | 93348-93655 |
| | | galactosaminyltransferase, polypeptide 2 | | |
| 310 | 3102 | nicotinamide phosphoribosyltransferase | 5.348 | 104902-105169 |
| 439 | 2903 | poly (ADP-ribose) polymerase family, member 1 | 23.907 | 141472-141718 |
| 676 | 2680 | fucosyltransferase 8 | 9.927 | 209841-210227 |
| 818 | 2576 | poly (ADP-ribose) polymerase family, member 8 | 6.624 | 253302-253609 |
| 1075 | 2439 | TCDD-inducible poly(ADP-ribose) polymerase | 8.079 | 337086-337454 |
| 1172 | 2394 | exostoses (multiple) 1 | 13.888 | 370087-370311 |
| 1284 | 2341 | WD repeat and FYVE domain containing 3 | 0.277 | 407115-407476 |
| 1580 | 2217 | beta 1,3-galactosyltransferase-like | 3.289 | 507336-507709 |
| 1671 | 2185 | phosphatidylinositol glycan anchor | 2.327 | 539094-539385 |
| | | biosynthesis, class Q | | |
| 1720 | 2167 | protein-O-mannosyltransferase 2 | 1.099 | 555946-556293 |
| 1813 | 2138 | poly (ADP-ribose) polymerase family, member 16 | 4.303 | 588191-588503 |
| 1869 | 2123 | O-linked N-acetylglucosamine (GlcNAc) | 0.839 | 607012-607348 |
| | | transferase (UDP-N- | | |
| | | acetylglucosamine:polypeptide-N- | | |
| | | acetylglucosaminyl transferase) | | |
| 1899 | 2113 | glycogen synthase 1, muscle | 2.695 | 617021-617381 |
| 1998 | 2081 | exostoses (multiple)-like 3 | 0.53 | 650808-651119 |
| 2056 | 2058 | liver glycogen phosphorylase | 4.632 | 670012-670314 |
| 2088 | 2048 | ST3 beta-galactoside alpha-2,3-sialyltransferase 1 | 5.651 | 681105-681454 |
| 2167 | 2021 | ST3 beta-galactoside alpha-2,3- | 13.01 | 707535-707870 |
| | | sialyltransferase 4 | | |
| 2174 | 2019 | brain glycogen phosphorylase | 3.301 | 709790-710087 |
| 2211 | 2008 | glycosyltransferase-like domain containing 1 | 3.796 | 722365-722668 |
| 2254 | 1995 | mannoside acetylglucosaminyltransferase 4, | 27.246 | 737377-737697 |
| | | isoenzyme B | | |
| 2363 | 1967 | exostoses (multiple) 2 | 12.067 | 774056-774354 |
| 2417 | 1954 | mannoside acetylglucosaminyltransferase 2 | 5.098 | 792371-792746 |
| 2557 | 1918 | UDP-glucose ceramide glucosyltransferase | 1.94 | 840181-840538 |
| 2589 | 1909 | UDP-Gal:betaGlcNAc beta 1,4- | 18.933 | 851115-851489 |
| | | galactosyltransferase, polypeptide 3 | | |
| 2597 | 1909 | UDP-GlcNAc:betaGal beta-1,3-N- | 2.935 | 853867-854128 |
| | | acetylglucosaminyltransferase 9 | | |
| 2696 | 1886 | glycosyltransferase 25 domain containing 1 | 29.095 | 886942-887213 |
| 2783 | 1861 | protein O-fucosyltransferase 2 | 28.156 | 916726-917035 |
| 2830 | 1851 | asparagine-linked glycosylation 12 homolog | 9.883 | 932756-933070 |
| | | (yeast, alpha-1,6-mannosyltransferase) | | |
| 2920 | 1824 | asparagine-linked glycosylation 8 homolog | 6.563 | 963558-963865 |
| | | (yeast, alpha-1,3-glucosyltransferase) | | |
| 3065 | 1776 | UDP-N-acetyl-alpha-D- | 1.546 | 1013002-1013376 |
| | | galactosamine:polypeptide N- | | |
| | | acetylgalactosaminyltransferase 10 | | |
| 3249 | 1736 | UDP-GlcNAc:betaGal beta-1,3-N- | 5.258 | 1075997-1076374 |
| | | acetylglucosaminyltransferase 2 | | |
| 3332 | 1709 | UDP-GlcNAc:betaGal beta-1,3-N- | 24.577 | 1104024-1104401 |
| | | acetylglucosaminyltransferase 1 | | |
| 3411 | 1689 | ST3 beta-galactoside alpha-2,3- | 3.964 | 1131123-1131445 |
| | | sialyltransferase 3 | | |
| 3472 | 1674 | glycogenin | 12.806 | 1151366-1151643 |
| 3484 | 1672 | ST3 beta-galactoside alpha-2,3- | 21.148 | 1155324-1155711 |
| | | sialyltransferase 5 | | |
| 3594 | 1646 | phosphatidylinositol glycan anchor | 0.64 | 1191982-1192311 |
| | | biosynthesis, class M | | |
| 3711 | 1621 | nicotinate phosphoribosyltransferase domain | 9.212 | 1231855-1232201 |
| | | containing 1 | | |
| 3731 | 1616 | glucan (1,4-alpha-), branching enzyme 1 | 2.847 | 1238609-1238920 |
| 3887 | 1577 | UDP-Gal:betaGlcNAc beta 1,4- | 5.414 | 1291326-1291668 |
| | | galactosyltransferase, polypeptide 2 | | |
| 3937 | 1565 | exostoses (multiple)-like 2 | 2.123 | 1307522-1307889 |
| 4007 | 1548 | protein-O-mannosyltransferase 1 | 1.418 | 1331135-1331436 |
| 4105 | 1522 | UDP-N-acetyl-alpha-D-galactosamine:polypeptide | 1.816 | 1363583-1363970 |
| | | N-acetylgalactosaminyltransferase 11 | | |
| 4177 | 1507 | RIKEN cDNA A130022J15 gene | 1.007 | 1387950-1388266 |
| 4186 | 1504 | ST6 (alpha-N-acetyl-neuraminyl-2,3-beta- | 5.237 | 1391079-1391449 |
| | | galactosyl-1,3)-N-acetylgalactosaminide | | |
| | | alpha-2,6-sialyltransferase 6 | | |
| 4319 | 1476 | ST3 beta-galactoside alpha-2,3-sialyltransferase 2 | 1.043 | 1435989-1436317 |
| 4391 | 1460 | dolichyl-phosphate (UDP-N- | 10.516 | 1460002-1460374 |
| | | acetylglucosamine) | | |
| | | acetylglucosaminephosphotransferase 1 | | |
| | | (GlcNAc-1-P transferase) | | |
| 4654 | 1402 | UDP-N-acetyl-alpha-D-galactosamine: | 0.782 | 1546609-1546999 |
| | | polypeptide N-acetylgalactosaminyltransferase 7 | | |
| 4671 | 1399 | UDP-Gal:betaGlcNAc beta 1,4- | 3.652 | 1552461-1552728 |
| | | galactosyltransferase, polypeptide 4 | | |
| 4673 | 1398 | phosphatidylinositol glycan anchor | 0.875 | 1553085-1553453 |
| | | biosynthesis, class V | | |
| 4701 | 1392 | UDP-Gal:betaGlcNAc beta 1,4- | 2.241 | 1562813-1563108 |
| | | galactosyltransferase, polypeptide 5 | | |
| 4795 | 1370 | asparagine-linked glycosylation 1 homolog | 4.698 | 1594394-1594762 |
| | | (yeast, beta-1,4-mannosyltransferase) | | |
| 4883 | 1350 | glycosyltransferase 8 domain containing 1 | 12.347 | 1624267-1624637 |
| 4914 | 1345 | UDP-Gal:betaGlcNAc beta 1,4- | 0.514 | 1635173-1635561 |
| | | galactosyltransferase, polypeptide 6 | | |
| 4945 | 1339 | mannoside acetylglucosaminyltransferase 5 | 0.5 | 1645857-1646201 |
| 5003 | 1323 | poly (ADP-ribose) polymerase family, member 6 | 1.689 | 1665747-1666131 |
| 5314 | 1256 | phosphatidylinositol glycan anchor | 1.768 | 1771843-1772168 |
| | | biosynthesis, class A | | |
| 5410 | 1235 | queuine tRNA-ribosyltransferase 1 | 3.554 | 1805877-1806240 |
| 5523 | 1206 | xylosylprotein beta1,4-galactosyltransferase, | 4.56 | 1845828-1846182 |
| | | polypeptide 7 (galactosyltransferase I) | | |
| 5541 | 1201 | phosphatidylinositol glycan anchor | 1.816 | 1852108-1852474 |
| | | biosynthesis, class C | | |
| 5577 | 1195 | poly (ADP-ribose) polymerase family, member 2 | 2.269 | 1864411-1864683 |
| 5594 | 1191 | mannoside acetylglucosaminyltransferase 1 | 3.072 | 1870192-1870557 |
| 5596 | 1190 | uridine monophosphate synthetase | 2.109 | 1870945-1871338 |
| 5603 | 1189 | like-glycosyltransferase | 1.088 | 1873387-1873696 |
| 5740 | 1158 | protein O-linked mannose beta1,2-N- | 2.323 | 1922712-1923111 |
| | | acetylglucosaminyltransferase | | |
| 5782 | 1148 | UDP-Gal:betaGal beta 1,3- | 2.721 | 1938009-1938394 |
| | | galactosyltransferase, polypeptide 6 | | |
| 5811 | 1143 | UDP-GalNAc:betaGlcNAc beta 1,3- | 1.658 | 1948459-1948787 |
| | | galactosaminyltransferase, polypeptide 1 | | |
| 6018 | 1098 | phosphatidylinositol glycan anchor | 0.881 | 2023895-2024261 |
| | | biosynthesis, class B | | |
| 6204 | 1057 | methylthioadenosine phosphorylase | 15.667 | 2091342-2091736 |
| 6220 | 1053 | asparagine-linked glycosylation 5 homolog (yeast, | 4.737 | 2097263-2097647 |
| | | dolichyl-phosphate beta-glucosyltransferase) | | |
| 6257 | 1043 | UDP-GlcNAc:betaGal beta-1,3-N- | 0.564 | 2110626-2111006 |
| | | acetylglucosaminyltransferase-like 1 | | |
| 6374 | 1019 | asparagine-linked glycosylation 11 homolog | 1.981 | 2151968-2152316 |
| | | (yeast, alpha-1,2-mannosyltransferase) | | |
| 6415 | 1008 | glycosyltransferase 8 domain containing 3 | 0.363 | 2166772-2167170 |
| 6428 | 1006 | core 1 synthase, glycoprotein-N- | 0.85 | 2171365-2171714 |
| | | acetylgalactosamine 3-β-galactosyltransferase, 1 | | |
| 6531 | 983 | hypoxanthine guanine phosphoribosyl | 40.474 | 2207724-2208109 |
| | | transferase 1 | | |
| 6806 | 924 | purine-nucleoside phosphorylase 1 | 10.99 | 2304356-2304474 |
| 6857 | 913 | protein O-fucosyltransferase 1 | 0.441 | 2321807-2322122 |
| 6893 | 904 | asparagine-linked glycosylation 2 homolog | 0.997 | 2332768-2333127 |
| | | (yeast, alpha-1,3-mannosyltransferase) | | |
| 6925 | 899 | dolichol-phosphate (beta-D) | 3.276 | 2343195-2343568 |
| | | mannosyltransferase 1 | | |
| 6955 | 891 | asparagine-linked glycosylation 9 homolog | 1.514 | 2353366-2353756 |
| | | (yeast, alpha 1,2 mannosyltransferase) | | |
| 7217 | 834 | poly (ADP-ribose) polymerase family, member 14 | 0.115 | 2440491-2440873 |
| 7484 | 767 | UDP-Gal:betaGlcNAc beta 1,4- | 0.387 | 2528454-2528763 |
| | | galactosyltransferase, polypeptide 1 | | |
| 7778 | 694 | RFNG O-fucosylpeptide 3-beta-N- | 1.377 | 2625536-2625911 |
| | | acetylglucosaminyltransferase | | |
| 7893 | 663 | phosphatidylinositol glycan anchor | 5.595 | 2664400-2664764 |
| | | biosynthesis, class P | | |
| 8007 | 632 | asparagine-linked glycosylation 6 homolog | 1.15 | 2702432-2702775 |
| | | (yeast, alpha-1,3,-glucosyltransferase) | | |
| 8072 | 608 | dolichol-phosphate (beta-D) | 1.511 | 2724089-2724407 |
| | | mannosyltransferase 2 | | |
| 8110 | 598 | LFNG O-fucosylpeptide 3-beta-N- | 0.277 | 2735576-2735965 |
| | | acetylglucosaminyltransferase | | |
| 8126 | 593 | fucosyltransferase 11 | 0.72 | 2740650-2740952 |
| 8137 | 591 | asparagine-linked glycosylation 13 homolog | 1.131 | 2744301-2744619 |
| | | (S. cerevisiae) | | |
| 8277 | 553 | adenine phosphoribosyl transferase | 7.251 | 2789152-2789451 |
| 8302 | 547 | poly (ADP-ribose) polymerase family, | 0.182 | 2797670-2797988 |
| | | member 11 | | |
| 8323 | 541 | ADP-ribosyltransferase 3 | 0.457 | 2804437-2804812 |
| 8510 | 493 | UDP-Gal:betaGlcNAc beta 1,3- | 0.099 | 2867869-2868208 |
| | | galactosyltransferase, polypeptide 1 | | |
| 8536 | 489 | UDP-N-acetyl-alpha-D- | 0.096 | 2876241-2876595 |
| | | galactosamine:polypeptide N- | | |
| | | acetylgalactosaminyltransferase 4 | | |
| 8900 | 417 | UDP glucuronosyltransferase 1 family, | 0.382 | 2990930-2991111 |
| | | polypeptide A6B | | |
| 9154 | 351 | UDP glucuronosyltransferase 1 family, | 0.106 | 3057120-3057211 |
| | | polypeptide A6A | | |
| 9275 | 322 | UDP-GlcNAc:betaGal beta-1,3-N- | 0.228 | 3083416-3083607 |
| | | acetylglucosaminyltransferase 4 | | |
| 3157421 | 431 | glycosyltransferase 8 domain containing 2 | 0.2 | 3173684-3173783 |
| 3157495 | 1014 | phosphatidylinositol glycan anchor biosynthesis, | 1.147 | 3183184-3183283 |
| | | class H | | |
| 3157929 | 501 | ADP-ribosyltransferase 2b | 0.579 | 3280549-3280648 |
| 3157944 | 155 | beta-1,4-N-acetyl-galactosaminyl transferase 2 | 0.038 | 3175384-3175483 |
| 3157960 | 2282 | ST8-N-acetyl-neuraminide-2,8-sialyltransferase 4 | 1.629 | 3246817-3246916 |
| 3158019 | 362 | ABO blood group (transferase A, 1-3-N- | 0.204 | 3185571-3185670 |
| | | acetylgalactosaminyltransferase, transferase B, 1- | | |
| | | 3-galactosyltransferase) | | |
| 3158211 | 343 | ST6 (-N-acetyl-neuraminyl-2,3-β-galactosyl-1,3)- | 0.282 | 3260605-3260704 |
| | | N-acetylgalactosaminide-2,6-sialyltransferase 4 | | |
| 3158222 | 726 | asparagine-linked glycosylation 10 homolog B | 0.262 | 3163121-3163220 |
| | | (yeast, -1,2-glucosyltransferase) | | |

TABLE 29
GTPase activators

| SEQ | | | Avg | siRNA SEQ |
|---|---|---|---|---|
| ID NO: | consL | Description | Cov | ID NOs: |
| 15 | 4557 | RIKEN cDNA B230339M05 gene | 3.965 | 14108-14438 |
| 58 | 4061 | TBC1 domain family, member 2B | 20.58 | 27984-28289 |
| 102 | 3754 | neurofibromatosis 1 | 1.523 | 42422-42742 |
| 128 | 3628 | regulator of G-protein signaling 17 | 3.266 | 50726-50999 |
| 231 | 3253 | SLIT-ROBO Rho GTPase activating protein 2 | 3.644 | 81642-81883 |
| 288 | 3130 | ArfGAP w/ SH# domain, ankyrin repeat & | 5.511 | 98329-98712 |
| | | PH domain 1 | | |
| 339 | 3047 | active BCR-related gene | 7.246 | 112574-112969 |
| 382 | 2979 | breakpoint cluster region | 3.754 | 125289-125540 |
| 385 | 2977 | GTPase activating RANGAP domain-like 1 | 1.897 | 126120-126355 |
| 422 | 2926 | Rho GTPase activating protein 18 | 15.948 | 136578-136825 |
| 469 | 2875 | ralA binding protein 1 | 6.921 | 149400-149662 |
| 574 | 2762 | GTPase activating protein & VPS9 domains 1 | 3.958 | 179030-179286 |
| 651 | 2697 | USP6 N-terminal like | 3.097 | 202215-202493 |
| 743 | 2635 | signal-induced proliferation-associated 1 like 1 | 5.005 | 230159-230551 |
| 766 | 2610 | Rho GTPase activating protein 21 | 4.9 | 236928-237164 |
| 872 | 2550 | ArfGAP with GTPase domain, ankyrin repeat | 0.806 | 269740-270071 |
| | | and PH domain 1 | | |
| 877 | 2547 | Rho GTPase activating protein 22 | 46.084 | 271221-271503 |
| 919 | 2530 | IQ motif containing GTPase activating protein 2 | 7.731 | 285026-285361 |
| 1013 | 2474 | ArfGAP with FG repeats 1 | 7.52 | 316623-316999 |
| 1019 | 2471 | TBC1 domain family, member 1 | 3.523 | 318888-319270 |
| 1021 | 2471 | Rho GTPase activating protein 24 | 7.769 | 319636-320032 |
| 1180 | 2390 | G protein-coupled receptor kinase-interactor 1 | 9.642 | 372750-373054 |
| 1202 | 2381 | G protein-coupled receptor kinase-interactor 2 | 1.721 | 380043-380305 |
| 1209 | 2378 | tuberous sclerosis 2 | 2.396 | 382192-382530 |
| 1231 | 2366 | ADP-ribosylation factor GTPase activating | 13 | 389186-389510 |
| | | protein 2 | | |
| 1237 | 2364 | rabaptin, RAB GTPase binding effector protein 1 | 1.86 | 391313-391594 |
| 1251 | 2357 | G-protein signalling modulator 2 (AGS3- | 25.263 | 396073-396448 |
| | | like, C. elegans) | | |
| 1391 | 2292 | ecotropic viral integration site 5 like | 2.064 | 443532-443866 |
| 1408 | 2285 | TBC1 domain family, member 15 | 5.501 | 449214-449575 |
| 1410 | 2285 | Rho GTPase activating protein 12 | 1.14 | 449921-450284 |
| 1449 | 2267 | guanosine diphosphate (GDP) dissociation | 25.652 | 463287-463618 |
| | | inhibitor 1 | | |
| 1479 | 2253 | TBC1 domain family, member 10b | 11.038 | 473445-473815 |
| 1513 | 2240 | RAN GTPase activating protein 1 | 12.173 | 484741-485095 |
| 1562 | 2226 | small G protein signaling modulator 3 | 9.371 | 501162-501548 |
| 1634 | 2197 | Rho GTPase activating protein 29 | 3.76 | 526292-526588 |
| 1642 | 2193 | IQ motif containing GTPase activating protein 1 | 0.799 | 529103-529460 |
| 1649 | 2191 | ADP-ribosylation factor GTPase activating | 17.61 | 531693-532043 |
| | | protein 1 | | |
| 1752 | 2158 | ArfGAP with GTPase domain, ankyrin repeat | 11.364 | 567066-567372 |
| | | and PH domain 3 | | |
| 1803 | 2141 | Rho GTPase activating protein 17 | 3.223 | 584731-585028 |
| 1858 | 2125 | TBC1 domain family, member 9B | 5.288 | 603350-603639 |
| 1886 | 2116 | ArfGAP with RhoGAP domain, ankyrin | 4.242 | 612818-613159 |
| | | repeat and PH domain 3 | | |
| 1922 | 2106 | stromal membrane-associated protein 1 | 12.305 | 624987-625343 |
| 1926 | 2104 | Rho guanine nucleotide exchange factor (GEF) 1 | 5.042 | 626255-626602 |
| 2031 | 2067 | TBC1 domain family, member 25 | 6.666 | 661569-661914 |
| 2165 | 2022 | RIKEN cDNA A230067G21 gene | 0.566 | 706803-707157 |
| 2223 | 2004 | Ras and Rab interactor 2 | 5.703 | 726472-726765 |
| 2289 | 1985 | amyotrophic lateral sclerosis 2 (juvenile) | 0.792 | 749132-749432 |
| | | homolog (human) | | |
| 2291 | 1985 | TBC1 domain family, member 17 | 6.336 | 749822-750199 |
| 2301 | 1983 | ADP-ribosylation factor GTPase activating | 5.309 | 753270-753612 |
| | | protein 3 | | |
| 2309 | 1981 | RAB GTPase activating protein 1-like | 1.389 | 755934-756259 |
| 2365 | 1966 | stromal membrane-associated GTPase- | 7.748 | 774678-775049 |
| | | activating protein 2 | | |
| 2419 | 1954 | RAB3 GTPase activating protein subunit 1 | 1.494 | 793063-793349 |
| 2479 | 1938 | oligophrenin 1 | 2.039 | 813214-813607 |
| 2534 | 1925 | signal-induced proliferation associated gene 1 | 3.696 | 832257-832632 |
| 2559 | 1917 | guanosine diphosphate (GDP) dissociation | 4.745 | 840859-841143 |
| | | inhibitor 2 | | |
| 2621 | 1902 | Rac GTPase-activating protein 1 | 19.316 | 861408-861766 |
| 2622 | 1902 | RAS p21 protein activator 3 | 2.103 | 861767-862055 |
| 2695 | 1886 | TBC1 domain family, member 22a | 1.294 | 886641-886941 |
| 2854 | 1845 | leucine-rich repeat kinase 2 | 1.495 | 940941-941325 |
| 2862 | 1841 | ArfGAP w/ coiled-coil, ankyrin repeat & PH | 1.693 | 943650-943952 |
| | | domains 2 | | |
| 3038 | 1783 | Rho GDP dissociation inhibitor (GDI) alpha | 85.766 | 1003934-1004232 |
| 3084 | 1771 | myosin IXb | 1.071 | 1019313-1019670 |
| 3134 | 1761 | resistance to inhibitors of cholinesterase 8 | 5.191 | 1036587-1036933 |
| | | homolog (C. elegans) | | |
| 3144 | 1759 | disabled homolog 2 (Drosophila) interacting | 1.484 | 1040220-1040553 |
| | | protein | | |
| 3163 | 1754 | rabaptin, RAB GTPase binding effector | 3.591 | 1046902-1047174 |
| | | protein 2 | | |
| 3512 | 1667 | RAS p21 protein activator 4 | 1.866 | 1164603-1164943 |
| 3637 | 1637 | Rho GTPase activating protein 25 | 4.095 | 1206771-1207157 |
| 3644 | 1635 | phosphatidylinositol-3,4,5-trisphosphate- | 2.011 | 1209078-1209429 |
| | | dependent Rac exchange factor 2 | | |
| 3676 | 1627 | ArfGAP with RhoGAP domain, ankyrin | 0.823 | 1219997-1220302 |
| | | repeat and PH domain 1 | | |
| 3750 | 1612 | SLIT-ROBO Rho GTPase activating protein 3 | 1.234 | 1245082-1245453 |
| 3760 | 1610 | TBC1 domain family, member 20 | 6.983 | 1248542-1248863 |
| 3805 | 1596 | signal-induced proliferation-associated 1 like 2 | 1.048 | 1263925-1264323 |
| 3902 | 1573 | GIPC PDZ domain containing family, | 31.917 | 1296244-1296541 |
| | | member 1 | | |
| 3911 | 1571 | TBC1 domain family, member 23 | 0.785 | 1299237-1299523 |
| 4133 | 1516 | ALS2 C-terminal like | 1.264 | 1373305-1373600 |
| 4479 | 1441 | DEP domain containing 1B | 2.389 | 1489111-1489393 |
| 4536 | 1428 | Rho GTPase activating protein 1 | 2.875 | 1507506-1507890 |
| 4552 | 1425 | Rho GTPase activating protein 6 | 0.435 | 1512969-1513333 |
| 4775 | 1373 | ecotropic viral integration site 5 | 1.536 | 1587335-1587660 |
| 4892 | 1348 | ADP-ribosylation factor-like 2 binding protein | 13.977 | 1627434-1627798 |
| 4971 | 1331 | WD repeat domain 67 | 0.743 | 1654864-1655263 |
| 5128 | 1294 | TBC1 domain family, member 10c | 1.46 | 1708143-1708504 |
| 5234 | 1274 | TBC1 domain family, member 4 | 0.291 | 1744511-1744853 |
| 5247 | 1272 | choroidermia | 0.842 | 1749109-1749507 |
| 5475 | 1218 | DEP domain containing 1a | 0.524 | 1828873-1829271 |
| 5704 | 1165 | Rho GTPase activating protein 10 | 4.456 | 1909622-1909976 |
| 5893 | 1127 | RIKEN cDNA 4933428G20 gene | 0.385 | 1978396-1978755 |
| 6057 | 1091 | TBC1 domain family, member 7 | 8.529 | 2037948-2038347 |
| 6189 | 1061 | SH3-domain binding protein 1 | 2.587 | 2085849-2086155 |
| 6387 | 1016 | development & differentiation enhancing factor 2 | 0.44 | 2156641-2157022 |
| 6449 | 1001 | TBC1 domain family, member 14 | 0.485 | 2178913-2179271 |
| 6597 | 969 | G-protein signalling modulator 3 (AGS3- | 10.243 | 2231290-2231663 |
| | | like, C. elegans) | | |
| 6629 | 963 | ankyrin repeat domain 27 (VPS9 domain) | 0.299 | 2242342-2242728 |
| 6789 | 928 | Rho GTPase activating protein 19 | 0.204 | 2298285-2298665 |
| 7012 | 877 | TBC1 domain family, member 24 | 0.285 | 2372442-2372763 |
| 7028 | 874 | T-cell lymphoma invasion and metastasis 2 | 0.333 | 2377813-2378151 |
| 7443 | 777 | TBC1 domain family, member 8 | 0.219 | 2515218-2515579 |
| 7553 | 749 | choroideremia-like | 0.203 | 2551113-2551507 |
| 7888 | 664 | StAR-related lipid transfer (START) domain | 0.12 | 2662630-2662978 |
| | | containing 13 | | |
| 7967 | 642 | RNA binding protein 1 | 2.033 | 2689665-2689951 |
| 8020 | 627 | Rho GTPase-activating protein | 0.115 | 2706942-2707263 |
| 8021 | 626 | RAS p21 protein activator 2 | 0.141 | 2707264-2707590 |
| 8342 | 537 | Rho GTPase activating protein 28 | 0.108 | 2811107-2811423 |
| 8393 | 522 | proline rich 5 (renal) | 0.462 | 2828643-2828993 |
| 8701 | 458 | TBC1 domain family, member 13 | 0.867 | 2930463-2930783 |
| 8702 | 458 | family with sequence similarity 13, member B | 0.273 | 2930784-2931124 |
| 8792 | 441 | RIKEN cDNA A630047E20 gene | 0.215 | 2959129-2959498 |
| 8865 | 425 | CDC42 GTPase-activating protein | 0.055 | 2980492-2980833 |
| 8990 | 396 | GTPase activating RANGAP domain-like 3 | 0.117 | 3015728-3016007 |
| 9085 | 369 | glucocorticoid receptor DNA binding factor 1 | 0.084 | 3040212-3040461 |
| 9146 | 354 | ArfGAP with FG repeats 2 | 0.239 | 3055161-3055411 |
| 9161 | 349 | RAB GTPase activating protein 1 | 0.083 | 3058415-3058689 |
| 9322 | 307 | Ras and Rab interactor 1 | 0.073 | 3093895-3094135 |
| 9483 | 252 | G-protein signalling modulator 1 (AGS3- | 0.075 | 3123987-3124129 |
| | | like, C. elegans) | | |
| 9653 | 186 | Rho GTPase activating protein 27 | 0.054 | 3144717-3144852 |
| 9665 | 179 | regulator of G-protein signaling 2 | 0.059 | 3145905-3146047 |
| 3157157 | 1019 | Rho guanine nucleotide exchange factor | 0.439 | 3188671-3188770 |
| | | (GEF) 19 | | |
| 3157282 | 1366 | regulator of G protein signaling 7 | 1.027 | 3244017-3244116 |
| 3157556 | 1034 | SLIT-ROBO Rho GTPase activating protein 1 | 0.398 | 3185171-3185270 |
| 3157624 | 439 | Rho GTPase activating protein 9 | 0.242 | 3186071-3186170 |
| 3157647 | 369 | Rho GTPase activating protein 20 | 0.059 | 3273896-3273995 |
| 3157800 | 319 | Ras and Rab interactor 3 | 0.082 | 3255305-3255404 |
| 3157893 | 356 | muscle-related coiled-coil protein | 0.173 | 3166984-3167083 |
| 3158205 | 1690 | TBC1D12: TBC1 domain family, member 12 | 2.618 | 3168384-3168483 |
| 3158329 | 1467 | synapse defective 1, Rho GTPase, homolog 1 | 1.092 | 3213858-3213957 |
| | | (C. elegans) | | |
| 3158404 | 1495 | NA | 0.581 | 3227117-3227216 |

TABLE 65
GTPases

| SEQ | | | Avg | siRNA SEQ |
|---|---|---|---|---|
| ID NO: | consL | Description | Covg | ID NOs: |
| 47 | 4181 | eukaryotic translation initiation factor 5B | 7.249 | 24507-24791 |
| 121 | 3653 | G1 to S phase transition 1 | 4.531 | 48461-48813 |
| 309 | 3103 | guanine nucleotide binding protein (G | 308.482 | 104658-104901 |
| | | protein), beta 1 | | |
| 333 | 3061 | guanine nucleotide binding protein (G | 12.233 | 110847-111128 |
| | | protein), alpha inhibiting 3 | | |
| 491 | 2850 | eukaryotic translation elongation factor 2 | 331.312 | 155612-155855 |
| 498 | 2844 | signal recognition particle receptor | 23.636 | 157648-157932 |
| | | (‘docking protein’) | | |
| 758 | 2618 | elongation factor Tu GTP binding domain | 10.74 | 234313-234699 |
| | | containing 1 | | |
| 869 | 2551 | Ras-related GTP binding C | 52.673 | 268890-269114 |
| 872 | 2550 | ArfGAP with GTPase domain, ankyrin | 0.806 | 269740-270071 |
| | | repeat and PH domain 1 | | |
| 874 | 2549 | elongation factor Tu GTP binding domain | 13.667 | 270373-270590 |
| | | containing 2 | | |
| 938 | 2520 | G elongation factor, mitochondrial 1 | 60.355 | 291757-292001 |
| 1104 | 2424 | ras homolog gene family, member Q | 4.127 | 346976-347213 |
| 1333 | 2322 | dynamin 2 | 10.975 | 423531-423830 |
| 1378 | 2296 | mitofusin 1 | 1.418 | 439108-439451 |
| 1397 | 2290 | RAS-related C3 botulinum substrate 1 | 73.806 | 445639-445879 |
| 1487 | 2250 | guanine nucleotide binding protein, alpha q | 1.455 | 476277-476517 |
| | | polypeptide | | |
| 1520 | 2239 | optic atrophy 1 homolog (human) | 2.52 | 487010-487405 |
| 1709 | 2172 | guanine nucleotide binding protein | 70.605 | 552132-552530 |
| | | (G protein), alpha inhibiting 2 | | |
| 1769 | 2154 | EH-domain containing 1 | 11.604 | 572945-573251 |
| 1816 | 2137 | dynamin 1-like | 4.171 | 589195-589429 |
| 1944 | 2097 | cell division cycle 42 homolog | 189.607 | 632324-632630 |
| | | (S. cerevisiae) | | |
| 2009 | 2076 | guanine nucleotide binding protein, 13 | 2.993 | 654543-654775 |
| 2124 | 2034 | ras homolog gene family, member A | 135.612 | 693012-693333 |
| 2488 | 1937 | GTP binding protein 2 | 5.681 | 816419-816817 |
| 2525 | 1927 | myxovirus (influenza virus) resistance 2 | 8.118 | 829145-829432 |
| 2560 | 1916 | EH-domain containing 2 | 3.355 | 841144-841487 |
| 2575 | 1913 | Hbs1-like (S. cerevisiae) | 2.621 | 846230-846577 |
| 2700 | 1885 | GUF1 GTPase homolog (S. cerevisiae) | 4.872 | 888158-888500 |
| 2834 | 1851 | neuroblastoma ras oncogene | 2.46 | 934198-934494 |
| 2857 | 1844 | guanine nucleotide binding protein, alpha | 2.474 | 942072-942447 |
| | | transducing 1 | | |
| 2918 | 1824 | eukaryotic translation initiation factor 2, | 14.911 | 962789-963172 |
| | | subunit 3, structural gene X-linked | | |
| 3009 | 1792 | eukaryotic elongation factor, | 3.035 | 994350-994678 |
| | | selenocysteine-tRNA-specific | | |
| 3041 | 1782 | GTP binding protein 1 | 3.109 | 1004869-1005198 |
| 3244 | 1737 | tubulin, alpha 1B | 543.754 | 1074476-1074632 |
| 3372 | 1701 | guanine nucleotide binding protein | 5.812 | 1117711-1118107 |
| | | (G protein), beta 4 | | |
| 3427 | 1687 | RAB5A, member RAS oncogene family | 21.29 | 1136305-1136633 |
| 3455 | 1678 | guanine nucleotide binding protein, alpha O | 0.792 | 1145689-1145997 |
| 3661 | 1631 | tubulin, beta 5 | 61.529 | 1214795-1215127 |
| 3670 | 1628 | atlastin GTPase 3 | 1.006 | 1217824-1218196 |
| 3715 | 1620 | tubulin, gamma 1 | 36.02 | 1233354-1233745 |
| 3812 | 1594 | tubulin, alpha 1A | 100.894 | 1266291-1266518 |
| 3829 | 1591 | guanine nucleotide binding protein | 74.137 | 1271846-1272244 |
| | | (G protein) β2 | | |
| 3862 | 1582 | guanine nucleotide binding protein, 11 | 4.154 | 1282881-1283160 |
| 3992 | 1550 | tubulin, 1C | 191.248 | 1326132-1326373 |
| 4044 | 1539 | tubulin, β2C | 81.933 | 1343483-1343875 |
| 4168 | 1509 | G elongation factor, mitochondrial 2 | 1.773 | 1384797-1385138 |
| 4180 | 1507 | mitofusin 2 | 4.551 | 1389006-1389340 |
| 4212 | 1498 | RAB5C, member RAS oncogene family | 34.285 | 1400104-1400434 |
| 4325 | 1476 | eukaryotic translation elongation factor 1 2 | 3.269 | 1437945-1438305 |
| 4398 | 1459 | tubulin, alpha 4A | 8.154 | 1462310-1462667 |
| 4458 | 1447 | GTP binding protein 3 | 3.549 | 1482368-1482685 |
| 4496 | 1437 | GNAS (guanine nucleotide binding protein, | 670.983 | 1494365-1494682 |
| | | alpha stimulating) complex locus | | |
| 4559 | 1425 | RAS-related protein-1a | 12.202 | 1515193-1515550 |
| 4689 | 1394 | mitochondrial translational initiation factor 2 | 1.08 | 1558510-1558896 |
| 4774 | 1373 | guanylate binding protein 2 | 1.175 | 1586947-1587334 |
| 4912 | 1345 | v-Ki-ras2 Kirsten rat sarcoma viral | 3.151 | 1634477-1634773 |
| | | oncogene homolog | | |
| 5185 | 1283 | Tu translation elongation factor, | 19.719 | 1727760-1728085 |
| | | mitochondrial | | |
| 5399 | 1238 | RAN, member RAS oncogene family | 61.287 | 1802120-1802418 |
| 5621 | 1186 | ras homolog gene family, member G | 3.894 | 1879861-1880245 |
| 5703 | 1165 | guanine nucleotide binding protein, 12 | 3.153 | 1909321-1909621 |
| 5909 | 1123 | RAB34, member of RAS oncogene family | 14.774 | 1984213-1984547 |
| 6358 | 1022 | EH-domain containing 3 | 1.748 | 2146156-2146534 |
| 6499 | 987 | guanine nucleotide binding protein | 0.658 | 2196424-2196754 |
| | | (G protein), inhibiting 1 | | |
| 6599 | 969 | epsilon-tubulin 1 | 0.387 | 2232061-2232442 |
| 6669 | 955 | ras homolog gene family, member U | 0.296 | 2256530-2256915 |
| 6843 | 915 | RAB13, member RAS oncogene family | 14.169 | 2316888-2317279 |
| 7557 | 748 | ADP-ribosylation factor related protein 1 | 2.613 | 2552598-2552994 |
| 7670 | 721 | myxovirus (influenza virus) resistance 1 | 0.687 | 2588615-2588951 |
| 7944 | 647 | ras homolog gene family, member J | 0.275 | 2681897-2682206 |
| 7975 | 639 | tubulin, beta 3 | 2.093 | 2692217-2692262 |
| 8248 | 561 | tubulin, beta 4 | 0.376 | 2779208-2779248 |
| 8318 | 541 | T-cell specific GTPase | 0.193 | 2802893-2803167 |
| 8330 | 540 | atlastin GTPase 2 | 0.154 | 2806902-2807275 |
| 8367 | 530 | ADP-ribosylation factor-like 4A | 0.247 | 2819826-2820225 |
| 8407 | 517 | guanine nucleotide binding protein, alpha | 0.16 | 2833728-2833995 |
| | | stimulating, olfactory type | | |
| 8423 | 514 | guanine nucleotide binding prot (G protein), β 3 | 0.305 | 2839381-2839711 |
| 8694 | 459 | tubulin, alpha 3A | 0.369 | 2928173-2928397 |
| 8711 | 456 | guanylate binding protein 5 | 0.254 | 2933557-2933829 |
| 8739 | 451 | tubulin, alpha 8 | 0.315 | 2942782-2943028 |
| 9004 | 392 | tubulin, beta 2A | 2.629 | 3019264-3019285 |
| 9250 | 330 | tubulin, beta 2B | 0.402 | 3078180-3078203 |
| 9400 | 283 | guanine nucl binding prot. (G protein), γ 3 | 0.168 | 3109391-3109586 |
| 9520 | 237 | RAS-like, family 2, locus 9 | 0.34 | 3129635-3129703 |
| 9605 | 205 | RAS-related C3 botulinum substrate 2 | 0.555 | 3140533-3140548 |
| 3157235 | 968 | Ras-like without CAAX 2 | 1.124 | 3191071-3191170 |
| 3157288 | 395 | RAB5B, member RAS oncogene family | 0.422 | 3248917-3249016 |
| 3157535 | 325 | interferon inducible GTPase 1 | 0.11 | 3164284-3164383 |
| 3157709 | 573 | guanine nucl binding prot, transducing 2 | 0.496 | 3265005-3265104 |
| 3157755 | 370 | interferon gamma induced GTPase | 0.182 | 3283049-3283148 |
| 3157887 | 312 | dynamin 3 | 0.068 | 3263705-3263804 |
| 3158025 | 336 | RAB37, member of RAS oncogene family | 0.154 | 3216491-3216590 |
| 3158055 | 477 | tubulin, alpha-like 3 | 0.154 | 3281749-3281848 |
| 3158311 | 545 | RAS, dexamethasone-induced 1 | 0.595 | 3272596-3272695 |

TABLE 66
Cytoskeleton

| SEQ | | | Avg | siRNA SEQ |
|---|---|---|---|---|
| ID NO: | consL | Description | Covg | ID NOs: |
| 19 | 4458 | platelet-activating factor acetylhydrolase, | 4.915 | 15430-15711 |
| | | isoform 1b, subunit 1 | | |
| 33 | 4278 | bicaudal D homolog 2 (Drosophila) | 8.144 | 19767-20155 |
| 38 | 4237 | hook homolog 3 (Drosophila) | 2.298 | 21465-21748 |
| 44 | 4201 | SH3-domain kinase binding protein 1 | 6.615 | 23443-23756 |
| 60 | 4019 | MYC binding protein 2 | 2.593 | 28599-28954 |
| 76 | 3941 | neuron navigator 1 | 0.619 | 34046-34322 |
| 125 | 3643 | eukaryotic translation initiation factor 3, | 40.603 | 49822-50120 |
| | | subunit A | | |
| 140 | 3579 | actinin, alpha 1 | 23.486 | 54297-54516 |
| 146 | 3553 | microtubule-actin crosslinking factor 1 | 3.329 | 56027-56372 |
| 165 | 3467 | radixin | 17.836 | 61786-62020 |
| 173 | 3427 | protein phosphatase 4, regulatory subunit 2 | 12.016 | 64291-64567 |
| 174 | 3423 | myosin phosphatase Rho interacting protein | 10.215 | 64568-64841 |
| 214 | 3308 | microtubule-associated protein, RP/EB | 9.685 | 76455-76767 |
| | | family, member 2 | | |
| 269 | 3164 | filamin, beta | 2.477 | 93056-93347 |
| 272 | 3152 | Rho-associated coiled-coil containing | 3.17 | 94052-94292 |
| | | protein kinase 1 | | |
| 278 | 3141 | abl-interactor 1 | 4.255 | 95601-95909 |
| 284 | 3135 | fermitin family homolog 2 (Drosophila) | 27.426 | 97199-97521 |
| 289 | 3129 | testis specific gene A14 | 3.473 | 98713-99031 |
| 301 | 3110 | MAP/microtubule affinity-regulating kinase 1 | 3.607 | 102310-102609 |
| 322 | 3079 | plectin 1 | 3.784 | 108038-108268 |
| 324 | 3075 | parvin, alpha | 6.971 | 108553-108801 |
| 380 | 2981 | topoisomerase (DNA) II binding protein 1 | 7.196 | 124761-125048 |
| 400 | 2954 | moesin | 56.571 | 130562-130798 |
| 408 | 2949 | protein Tyr phosphatase, non-receptor type 14 | 0.65 | 132677-132944 |
| 446 | 2899 | actinin alpha 4 | 23.469 | 143309-143623 |
| 451 | 2894 | kinetochore associated 1 | 2.501 | 144746-145029 |
| 477 | 2865 | spindlin 1 | 18.581 | 151421-151677 |
| 480 | 2863 | erythrocyte protein band 4.1-like 2 | 9.783 | 152372-152645 |
| 505 | 2836 | myosin, heavy polypeptide 9, non-muscle | 1.62 | 159627-159938 |
| 508 | 2836 | filamin, alpha | 21.729 | 160437-160654 |
| 518 | 2828 | FERM domain containing 4A | 2.061 | 163151-163399 |
| 527 | 2815 | spectrin beta 2 | 2.13 | 165820-166071 |
| 528 | 2814 | spastin | 4.005 | 166072-166288 |
| 543 | 2791 | Janus kinase 2 | 4.149 | 170408-170768 |
| 562 | 2773 | uveal autoantigen with coiled-coil domains | 14.958 | 175535-175851 |
| | | and ankyrin repeats | | |
| 573 | 2763 | calmodulin 1 | 15.152 | 178775-179029 |
| 581 | 2754 | tubulin, gamma complex associated protein 5 | 3.189 | 181260-181476 |
| 617 | 2725 | dynactin 1 | 7.088 | 191583-191944 |
| 621 | 2718 | catenin (cadherin associated protein), alpha 1 | 30.996 | 192742-193116 |
| 659 | 2690 | kinesin family member 15 | 9.379 | 204514-204832 |
| 664 | 2688 | tetratricopeptide repeat domain 21B | 5.893 | 205946-206342 |
| 666 | 2688 | adducin 1 (alpha) | 12.01 | 206742-207131 |
| 706 | 2661 | retinoic acid induced 14 | 5.867 | 218315-218707 |
| 730 | 2639 | microtubule associated serine/threonine kinase 2 | 9.069 | 225909-226275 |
| 749 | 2626 | WD repeat domain 1 | 18.988 | 231778-232036 |
| 763 | 2614 | capping protein (actin filament) muscle Z- | 5.404 | 235917-236159 |
| | | line, alpha 1 | | |
| 766 | 2610 | Rho GTPase activating protein 21 | 4.9 | 236928-237164 |
| 806 | 2584 | spermatid perinuclear RNA binding protein | 1.175 | 249509-249890 |
| 811 | 2582 | ajuba | 12.735 | 251195-251502 |
| 812 | 2581 | outer dense fiber of sperm tails 2 | 4.42 | 251503-251729 |
| 830 | 2569 | Fgfr1 oncogene partner | 3.18 | 256961-257251 |
| 831 | 2569 | microtubule-associated protein 7 domain | 16.621 | 257252-257564 |
| | | containing 1 | | |
| 841 | 2564 | kinesin family member 5B | 3.218 | 260377-260685 |
| 848 | 2561 | ARP1 actin-related protein 1 homolog A, | 27.559 | 262622-262828 |
| | | centractin alpha (yeast) | | |
| 860 | 2555 | cortactin | 45.514 | 265970-266319 |
| 889 | 2543 | katanin p80 (WD40-containing) subunit B 1 | 12.112 | 275290-275634 |
| 905 | 2536 | ADP-ribosylation factor-like 8B | 2.122 | 280457-280707 |
| 962 | 2503 | expressed sequence AW555464 | 2.283 | 299312-299692 |
| 972 | 2498 | ARP3 actin-related protein 3 homolog (yeast) | 166.603 | 302483-302872 |
| 993 | 2485 | ARP2 actin-related protein 2 homolog (yeast) | 26.066 | 309842-310230 |
| 997 | 2483 | abl-interactor 2 | 1.097 | 311332-311712 |
| 1006 | 2477 | large tumor suppressor 2 | 3.379 | 314156-314545 |
| 1021 | 2471 | Rho GTPase activating protein 24 | 7.769 | 319636-320032 |
| 1026 | 2466 | FERM domain containing 4B | 2.223 | 321426-321672 |
| 1062 | 2447 | septin 2 | 12.767 | 333080-333462 |
| 1069 | 2440 | dishevelled, dsh homolog 1 (Drosophila) | 21.381 | 335136-335489 |
| 1081 | 2436 | ezrin | 31.498 | 339220-339540 |
| 1085 | 2434 | Wiskott-Aldrich syndrome-like (human) | 1.492 | 340449-340691 |
| 1094 | 2430 | SCY1-like 1 (S. cerevisiae) | 12.863 | 343589-343905 |
| 1097 | 2428 | sarcolemma associated protein | 1.377 | 344524-344917 |
| 1103 | 2426 | dystonin | 1.863 | 346599-346975 |
| 1117 | 2418 | drebrin 1 | 25.781 | 351269-351567 |
| 1126 | 2412 | spectrin alpha 2 | 4.506 | 354395-354680 |
| 1133 | 2410 | Rpgrip1-like | 0.564 | 357006-357378 |
| 1175 | 2393 | inositol polyphosphate phosphatase-like 1 | 3.628 | 371083-371386 |
| 1189 | 2386 | talin 1 | 2.605 | 375983-376311 |
| 1208 | 2379 | CDC14 cell division cycle 14 homolog A | 2.141 | 381807-382191 |
| | | (S. cerevisiae) | | |
| 1247 | 2359 | microtubule-associated protein, RP/EB | 18.63 | 394632-394981 |
| | | family, member 1 | | |
| 1255 | 2354 | centrosomal protein 110 | 0.814 | 397494-397774 |
| 1278 | 2344 | FERM domain containing 6 | 4.1 | 404997-405390 |
| 1279 | 2343 | TRIO and F-actin binding protein | 34.395 | 405391-405739 |
| 1296 | 2335 | amyloid beta (A4) precursor protein-binding, | 13.338 | 411043-411389 |
| | | family B, member 1 interacting protein | | |
| 1314 | 2327 | centrosomal protein 135 | 0.683 | 417267-417520 |
| 1327 | 2324 | centrosomal protein 55 | 19.363 | 421520-421872 |
| 1333 | 2322 | dynamin 2 | 10.975 | 423531-423830 |
| 1349 | 2315 | erythrocyte protein band 4.1-like 1 | 3.165 | 429287-429612 |
| 1353 | 2311 | centrosomal protein 63 | 8.32 | 430642-430998 |
| 1359 | 2308 | smoothelin | 3.137 | 432687-433041 |
| 1366 | 2303 | cofilin 2, muscle | 5.323 | 435213-435610 |
| 1392 | 2291 | tubulin folding cofactor E-like | 2.632 | 443867-444266 |
| 1404 | 2287 | A kinase (PRKA) anchor protein (gravin) 12 | 7.444 | 447806-448127 |
| 1434 | 2273 | expressed sequence AI314180 | 9.004 | 458166-458524 |
| 1458 | 2262 | Alstrom syndrome 1 homolog (human) | 0.712 | 466342-466731 |
| 1466 | 2259 | coiled-coil domain containing 85B | 25.622 | 469052-469416 |
| 1505 | 2243 | growth arrest-specific 2 like 1 | 14.146 | 482255-482606 |
| 1506 | 2243 | WAS/WASL interacting protein family, | 5.215 | 482607-482938 |
| | | member 1 | | |
| 1513 | 2240 | RAN GTPase activating protein 1 | 12.173 | 484741-485095 |
| 1519 | 2239 | KRIT1, ankyrin repeat containing | 9.236 | 486770-487009 |
| 1530 | 2238 | tropomodulin 3 | 3.292 | 490452-490783 |
| 1531 | 2237 | twinfilin, actin-binding protein, homolog 1 | 14.634 | 490784-491124 |
| | | (Drosophila) | | |
| 1557 | 2227 | annexin A11 | 55.567 | 499580-499921 |
| 1558 | 2227 | parvin, beta | 4.466 | 499922-500228 |
| 1565 | 2224 | ZW10 homolog (Drosophila), | 12.629 | 502292-502621 |
| | | centromere/kinetochore protein | | |
| 1566 | 2224 | coronin, actin binding protein 1C | 4.605 | 502622-502971 |
| 1577 | 2218 | transforming, acidic coiled-coil containing | 1.821 | 506354-506697 |
| | | protein 2 | | |
| 1582 | 2214 | family with sequence similarity 83, member D | 12.849 | 508106-508316 |
| 1593 | 2210 | rho/rac guanine nucleotide exchange factor | 3.451 | 511846-512237 |
| | | (GEF) 2 | | |
| 1610 | 2205 | ankyrin 2, brain | 0.639 | 517928-518264 |
| 1673 | 2184 | adducin 3 (gamma) | 5.724 | 539781-540145 |
| 1682 | 2180 | microtubule associated monoxygenase, | 6.737 | 542784-543124 |
| | | calponin and LIM domain containing 1 | | |
| 1767 | 2154 | programmed cell death 6 interacting protein | 24.668 | 572196-572546 |
| 1820 | 2137 | slingshot homolog 3 (Drosophila) | 2.567 | 590404-590772 |
| 1831 | 2134 | dystroglycan 1 | 3.205 | 594147-594505 |
| 1850 | 2128 | nephronophthisis 4 (juvenile) homolog (human) | 2.545 | 600625-600911 |
| 1886 | 2116 | ArfGAP with RhoGAP domain, ankyrin | 4.242 | 612818-613159 |
| | | repeat and PH domain 3 | | |
| 1903 | 2112 | nuclear distribution gene E-like homolog 1 | 8.837 | 618380-618725 |
| | | (A. nidulans) | | |
| 1910 | 2110 | macrophage erythroblast attacher | 48.23 | 620748-621108 |
| 1939 | 2098 | leucine zipper, putative tumor suppressor 2 | 14.187 | 630655-630915 |
| 1940 | 2098 | kinesin family member C3 | 6.785 | 630916-631256 |
| 2010 | 2075 | myosin XVIIIA | 1.283 | 654776-655088 |
| 2020 | 2072 | vasodilator-stimulated phosphoprotein | 13.006 | 658006-658374 |
| 2023 | 2072 | zyxin | 12.684 | 658946-659253 |
| 2033 | 2066 | nucleoporin 85 | 17.448 | 662254-662568 |
| 2050 | 2061 | engulfment and cell motility 2, ced-12 | 7.176 | 668000-668354 |
| | | homolog (C. elegans) | | |
| 2095 | 2045 | CAP, adenylate cyclase-associated protein 1 | 88.915 | 683551-683847 |
| | | (yeast) | | |
| 2105 | 2042 | CD2-associated protein | 0.744 | 686855-687170 |
| 2124 | 2034 | ras homolog gene family, member A | 135.612 | 693012-693333 |
| 2136 | 2031 | midline 2 | 0.659 | 697033-697389 |
| 2160 | 2024 | lethal giant larvae homolog 1 (Drosophila) | 2.293 | 705082-705399 |
| 2175 | 2018 | dishevelled 2, dsh homolog (Drosophila) | 3.722 | 710088-710457 |
| 2191 | 2013 | ARP8 actin-related protein 8 homolog | 8.289 | 715461-715836 |
| | | (S. cerevisiae) | | |
| 2204 | 2010 | actin filament associated protein 1 | 1.126 | 719890-720203 |
| 2205 | 2010 | CDC42 effector protein (Rho | 4.544 | 720204-720579 |
| | | GTPase binding) 1 | | |
| 2212 | 2008 | thyroid hormone receptor interactor 10 | 30.196 | 722669-723012 |
| 2220 | 2005 | tropomyosin 4 | 428.406 | 725519-725834 |
| 2232 | 2001 | gene model 114 | 3.412 | 729587-729910 |
| 2235 | 2001 | septin 7 | 3.112 | 730587-730976 |
| 2295 | 1984 | microcephaly, primary autosomal recessive 1 | 0.629 | 751244-751582 |
| 2346 | 1973 | calmodulin 3 | 14.014 | 768392-768693 |
| 2354 | 1970 | protein phosphatase 1, regulatory subunit 9B | 2.194 | 771093-771432 |
| 2375 | 1964 | amyloid beta precursor protein (cytoplasmic | 13.369 | 778032-778283 |
| | | tail) binding protein 2 | | |
| 2379 | 1963 | protein regulator of cytokinesis 1 | 14.63 | 779205-779513 |
| 2387 | 1962 | intraflagellar transport 80 homolog | 0.991 | 782001-782399 |
| | | (Chlamydomonas) | | |
| 2416 | 1954 | kinesin family member C1 | 16.341 | 792040-792370 |
| 2430 | 1949 | anillin, actin binding protein | 2.848 | 796726-797054 |
| 2441 | 1946 | CLIP associating protein 2 | 1.013 | 800461-800731 |
| 2466 | 1941 | centrosomal protein 170 | 0.772 | 808772-809083 |
| 2479 | 1938 | oligophrenin 1 | 2.039 | 813214-813607 |
| 2482 | 1938 | leucine rich repeat containing 49 | 3.959 | 814326-814699 |
| 2506 | 1931 | Mid1 interacting protein 1 (gastrulation | 129.96 | 822665-823028 |
| | | specific G12-like (zebrafish)) | | |
| 2510 | 1930 | Bardet-Biedl syndrome 4 (human) | 5.356 | 824110-824394 |
| 2512 | 1930 | formin homology 2 domain containing 1 | 2.963 | 824760-825066 |
| 2520 | 1929 | drebrin-like | 40.695 | 827385-827727 |
| 2543 | 1922 | beclin 1, autophagy related | 22.681 | 835365-835694 |
| 2546 | 1921 | actin, gamma, cytoplasmic 1 | 284.261 | 836348-836704 |
| 2551 | 1919 | coiled-coil and C2 domain containing 2A | 0.604 | 838097-838446 |
| 2578 | 1912 | hook homolog 2 (Drosophila) | 4.1 | 847312-847598 |
| 2583 | 1910 | inner centromere protein | 4.499 | 848988-849386 |
| 2605 | 1907 | protein serine kinase H1 | 2.142 | 856267-856572 |
| 2609 | 1905 | Janus kinase 1 | 7.769 | 857488-857805 |
| 2621 | 1902 | Rac GTPase-activating protein 1 | 19.316 | 861408-861766 |
| 2644 | 1897 | protein phosphatase 2 (formerly 2A), | 46.955 | 869071-869380 |
| | | catalytic subunit, alpha isoform | | |
| 2691 | 1887 | growth arrest specific 2 | 2.282 | 885284-885579 |
| 2745 | 1872 | mitotic arrest deficient 1-like 1 | 4.132 | 903571-903958 |
| 2764 | 1866 | DDB1 and CUL4 associated factor 12 | 5.371 | 910380-910622 |
| 2775 | 1862 | actin, beta | 57.391 | 913994-914315 |
| 2805 | 1856 | enabled homolog (Drosophila) | 2.768 | 924287-924598 |
| 2816 | 1854 | coronin, actin binding protein 1B | 56.328 | 928073-928458 |
| 2841 | 1849 | tubulin, gamma complex associated protein 3 | 3.057 | 936562-936886 |
| 2844 | 1847 | large tumor suppressor | 0.394 | 937654-937969 |
| 2880 | 1837 | actin related protein 2/3 complex, subunit 5 | 60.269 | 949749-950130 |
| 2896 | 1832 | centromere protein E | 1.871 | 955437-955745 |
| 2943 | 1818 | LIM and SH3 protein 1 | 13.57 | 971615-971919 |
| 3042 | 1782 | sphingosine-1-phosphate phosphatase 1 | 3.922 | 1005199-1005578 |
| 3050 | 1780 | centrosomal protein 68 | 0.822 | 1007927-1008310 |
| 3069 | 1775 | centlein, centrosomal protein | 0.588 | 1014347-1014609 |
| 3082 | 1772 | pleckstrin homology domain containing, | 3.954 | 1018621-1018991 |
| | | family H (with MyTH4 domain) member 3 | | |
| 3084 | 1771 | myosin IXb | 1.071 | 1019313-1019670 |
| 3104 | 1768 | capping protein (actin filament) muscle Z- | 15.011 | 1026343-1026702 |
| | | line, 2 | | |
| 3147 | 1758 | dynein cytoplasmic 2 heavy chain 1 | 0.317 | 1041205-1041524 |
| 3170 | 1752 | PTK2 protein tyrosine kinase 2 | 5.096 | 1049366-1049709 |
| 3172 | 1752 | FYVE, RhoGEF and PH domain containing 1 | 5.286 | 1050013-1050360 |
| 3199 | 1747 | vimentin | 514.871 | 1059326-1059717 |
| 3207 | 1744 | ring finger protein 19A | 1.513 | 1062111-1062496 |
| 3211 | 1742 | phosphodiesterase 4D interacting prot | 1.285 | 1063460-1063768 |
| | | (myomegalin) | | |
| 3215 | 1742 | c-abl oncogene 1, receptor tyrosine kinase | 0.436 | 1064790-1065134 |
| 3223 | 1741 | CDC42 effector prot (Rho GTPase binding) 3 | 6.317 | 1067503-1067844 |
| 3230 | 1739 | destrin | 50.913 | 1069789-1070099 |
| 3263 | 1730 | tubulin-specific chaperone E | 13.488 | 1080945-1081272 |
| 3306 | 1717 | CLIP associating protein 1 | 0.948 | 1095379-1095748 |
| 3341 | 1708 | sorbin and SH3 domain containing 3 | 7.794 | 1107024-1107409 |
| 3502 | 1669 | microtubule-associated protein 6 | 3.649 | 1161307-1161624 |
| 3505 | 1668 | katanin p60 (ATPase-containing) subunit A1 | 32.182 | 1162218-1162611 |
| 3541 | 1661 | membrane protein, palmitoylated | 15.267 | 1174530-1174867 |
| 3577 | 1650 | cell division cycle 25 homolog B (S. pombe) | 1.866 | 1186395-1186715 |
| 3583 | 1649 | checkpoint kinase 1 homolog (S. pombe) | 3.146 | 1188354-1188736 |
| 3590 | 1647 | capping protein (actin filament) muscle Z- | 60.716 | 1190654-1190998 |
| | | line, beta | | |
| 3593 | 1647 | serologically defined colon cancer antigen 8 | 3.621 | 1191627-1191981 |
| 3609 | 1642 | tubulin, delta 1 | 13.501 | 1197043-1197421 |
| 3643 | 1635 | metastasis suppressor 1 | 0.4 | 1208709-1209077 |
| 3692 | 1625 | family with sequence similarity 82, member A2 | 4.761 | 1225295-1225616 |
| 3715 | 1620 | tubulin, gamma 1 | 36.02 | 1233354-1233745 |
| 3720 | 1619 | CDK5 regulatory subunit associated protein 2 | 1.713 | 1235062-1235355 |
| 3724 | 1618 | catenin (cadherin associated protein), -like 1 | 0.699 | 1236365-1236728 |
| 3774 | 1605 | family with sequence similarity 110, | 2.345 | 1253240-1253580 |
| | | member B | | |
| 3781 | 1603 | profilin 2 | 23.497 | 1255751-1256078 |
| 3796 | 1599 | phosphatidylinositol transfer protein, | 0.365 | 1260891-1261174 |
| | | membrane-associated 2 | | |
| 3846 | 1585 | centrosomal protein 72 | 5.434 | 1277509-1277820 |
| 3850 | 1585 | actin related protein 2/3 complex, subunit 1A | 46.114 | 1278978-1279372 |
| 3898 | 1574 | twinfilin, actin-binding protein, homolog 2 | 27.133 | 1295102-1295393 |
| | | (Drosophila) | | |
| 3901 | 1574 | FYVE, RhoGEF and PH domain containing 6 | 0.595 | 1295966-1296243 |
| 3910 | 1572 | cyclin B1 | 25.641 | 1298863-1299236 |
| 3933 | 1566 | ARP10 actin-related protein 10 homolog | 11.257 | 1306422-1306806 |
| | | (S. cerevisiae) | | |
| 3946 | 1562 | polo-like kinase 4 (Drosophila) | 2.986 | 1310666-1311034 |
| 3949 | 1562 | Ena-vasodilator stimulated phosphoprotein | 3.874 | 1311646-1311940 |
| 4009 | 1547 | ELMO domain containing 2 | 0.601 | 1331721-1332074 |
| 4014 | 1545 | protein phosphatase 2 (formerly 2A), | 82.162 | 1333415-1333732 |
| | | catalytic subunit, beta isoform | | |
| 4017 | 1545 | Janus kinase 3 | 1.252 | 1334368-1334721 |
| 4036 | 1541 | diaphanous homolog 1 (Drosophila) | 1.436 | 1340818-1341199 |
| 4088 | 1525 | ectodermal-neural cortex 1 | 2.166 | 1357871-1358264 |
| 4103 | 1522 | HAUS augmin-like complex, subunit 4 | 20.991 | 1362890-1363204 |
| 4160 | 1510 | fibronectin type 3 and SPRY domain- | 2.066 | 1382212-1382607 |
| | | containing protein | | |
| 4163 | 1510 | glycophorin C | 7.299 | 1383318-1383614 |
| 4176 | 1507 | WASP family 1 | 1.25 | 1387619-1387949 |
| 4180 | 1507 | mitofusin 2 | 4.551 | 1389006-1389340 |
| 4181 | 1507 | protein Tyr phosphatase, non-receptor type 13 | 0.677 | 1389341-1389646 |
| 4199 | 1500 | cytoskeleton associated protein 2 | 1.674 | 1395624-1396011 |
| 4202 | 1500 | intraflagellar transport 57 homolog | 4.102 | 1396618-1396929 |
| | | (Chlamydomonas) | | |
| 4220 | 1496 | centrosomal protein 57 | 2.62 | 1402802-1403129 |
| 4238 | 1493 | nucleoporin 62 | 5.816 | 1408710-1409085 |
| 4239 | 1493 | tripartite motif-containing 54 | 10.739 | 1409086-1409394 |
| 4251 | 1492 | UBX domain protein 6 | 21.107 | 1412861-1413233 |
| 4257 | 1491 | LIM domain and actin binding 1 | 0.916 | 1414950-1415263 |
| 4260 | 1489 | TRAF3 interacting protein 1 | 1.646 | 1415915-1416305 |
| 4285 | 1483 | dynactin 4 | 1.16 | 1424542-1424937 |
| 4370 | 1466 | shroom family member 3 | 0.482 | 1452845-1453240 |
| 4386 | 1462 | growth arrest specific 8 | 4.02 | 1458247-1458599 |
| 4408 | 1457 | influenza virus NS1A binding protein | 0.809 | 1465620-1465977 |
| 4457 | 1447 | erythrocyte protein band 4.1 | 0.529 | 1481994-1482367 |
| 4484 | 1440 | sarcoglycan, epsilon | 14.957 | 1490848-1491203 |
| 4498 | 1437 | slingshot homolog 1 (Drosophila) | 1.043 | 1494976-1495267 |
| 4503 | 1435 | ARP1 actin-related protein 1 homolog B, | 3.69 | 1496661-1496992 |
| | | centractin beta (yeast) | | |
| 4550 | 1426 | PDZ and LIM domain 1 (elfin) | 53.065 | 1512325-1512634 |
| 4552 | 1425 | Rho GTPase activating protein 6 | 0.435 | 1512969-1513333 |
| 4564 | 1423 | paxillin | 2.436 | 1517047-1517389 |
| 4570 | 1422 | coactosin-like 1 (Dictyostelium) | 22.98 | 1519097-1519422 |
| 4604 | 1415 | CAP-GLY domain containing linker protein 2 | 1.613 | 1530649-1531015 |
| 4612 | 1413 | cysteine and glycine-rich protein 1 | 17.093 | 1533366-1533652 |
| 4616 | 1412 | microtubule associated monoxygenase, | 0.37 | 1534571-1534894 |
| | | calponin and LIM domain containing-like 1 | | |
| 4667 | 1399 | family with sequence similarity 110, | 4.673 | 1551090-1551435 |
| | | member A | | |
| 4729 | 1387 | regulator of chromosome condensation 2 | 9.39 | 1571986-1572324 |
| 4732 | 1386 | sirtuin 2 (silent mating type information | 9.325 | 1573015-1573411 |
| | | regulation 2, homolog) 2 (S. cerevisiae) | | |
| 4775 | 1373 | ecotropic viral integration site 5 | 1.536 | 1587335-1587660 |
| 4778 | 1373 | tropomyosin 1, alpha | 14.432 | 1588306-1588667 |
| 4811 | 1366 | coiled-coil domain containing 99 | 1.214 | 1599899-1600288 |
| 4852 | 1357 | syntrophin, basic 2 | 0.315 | 1614004-1614358 |
| 4869 | 1354 | transforming growth factor beta 1 induced | 2.305 | 1619537-1619815 |
| | | transcript 1 | | |
| 4892 | 1348 | ADP-ribosylation factor-like 2 binding protein | 13.977 | 1627434-1627798 |
| 4903 | 1347 | tyrosine kinase 2 | 0.405 | 1631375-1631670 |
| 4907 | 1346 | CDC42 small effector 2 | 1.468 | 1632810-1633100 |
| 4913 | 1345 | ninein-like | 0.788 | 1634774-1635172 |
| 4941 | 1339 | catenin (cadherin associated protein), beta 1 | 0.495 | 1644372-1644747 |
| 4956 | 1336 | ADP-ribosylation factor-like 6 interacting | 32.187 | 1649516-1649856 |
| | | protein 5 | | |
| 4987 | 1326 | actin related protein 2/3 complex, subunit 4 | 80.61 | 1660460-1660839 |
| 5010 | 1321 | protein phosphatase 4, catalytic subunit | 90.194 | 1668127-1668488 |
| 5024 | 1319 | pre-B-cell leukemia transcription factor | 2.045 | 1672920-1673252 |
| | | interacting protein 1 | | |
| 5066 | 1310 | centrosomal protein 97 | 0.234 | 1687017-1687411 |
| 5084 | 1303 | Sfi1 homolog, spindle assembly associated | 0.686 | 1693415-1693807 |
| | | (yeast) | | |
| 5091 | 1302 | proline-serine-threonine phosphatase- | 12.697 | 1695903-1696267 |
| | | interacting protein 1 | | |
| 5104 | 1300 | nuclear distribution gene C homolog | 102.462 | 1699971-1700369 |
| | | (Aspergillus) | | |
| 5108 | 1299 | actin, alpha 2, smooth muscle, aorta | 3.284 | 1701331-1701660 |
| 5171 | 1285 | fuzzy homolog (Drosophila) | 6.367 | 1722855-1723213 |
| 5186 | 1283 | neurofibromatosis 2 | 0.719 | 1728086-1728462 |
| 5198 | 1282 | centrosomal protein 120 | 2.153 | 1732348-1732733 |
| 5208 | 1279 | nucleolar and spindle associated protein 1 | 2.386 | 1735724-1736042 |
| 5253 | 1270 | dynein cytoplasmic 2 light intermediate chain 1 | 9.834 | 1750866-1751258 |
| 5274 | 1266 | protein Tyr phosphatase, non-receptor type 21 | 0.472 | 1758166-1758517 |
| 5370 | 1243 | HAUS augmin-like complex, subunit 7 | 59.234 | 1791926-1792280 |
| 5402 | 1237 | myristoylated Ala rich protein kinase C | 3.148 | 1803092-1803482 |
| | | substrate | | |
| 5448 | 1226 | RIKEN cDNA F630043A04 gene | 2.085 | 1819143-1819511 |
| 5503 | 1212 | stomatin (Epb7.2)-like 2 | 17.579 | 1838816-1839204 |
| 5654 | 1177 | SMEK homolog 1, suppressor of mek1 | 0.871 | 1891648-1892008 |
| | | (Dictyostelium) | | |
| 5678 | 1171 | actin related protein 2/3 complex, subunit 2 | 15.986 | 1900301-1900676 |
| 5698 | 1166 | aurora kinase A | 16.855 | 1907469-1907831 |
| 5701 | 1166 | telomeric repeat binding factor 1 | 2.789 | 1908582-1908967 |
| 5716 | 1164 | cofilin 1, non-muscle | 107.826 | 1914036-1914356 |
| 5771 | 1151 | TNFRSF1A-associated via death domain | 11.061 | 1934043-1934332 |
| 5773 | 1151 | protein tyrosine phosphatase 4a1 | 0.279 | 1934685-1935079 |
| 5774 | 1151 | centrobin, centrosomal BRCA2 interacting prot | 1.021 | 1935080-1935410 |
| 5785 | 1148 | dynactin 5 | 5.2 | 1939165-1939526 |
| 5791 | 1147 | microtubule-associated protein 1S | 6.328 | 1941401-1941793 |
| 5864 | 1133 | kinesin family member 18A | 1.465 | 1967839-1968177 |
| 5882 | 1129 | calmodulin 2 | 263.807 | 1974401-1974748 |
| 5943 | 1115 | PDZ and LIM domain 7 | 17.513 | 1996784-1997110 |
| 5944 | 1115 | serine/threonine kinase 38 like | 0.267 | 1997111-1997478 |
| 6060 | 1089 | cell division cycle associated 8 | 7.204 | 2039068-2039461 |
| 6067 | 1087 | sorbin and SH3 domain containing 1 | 1.201 | 2041684-2042038 |
| 6068 | 1087 | tropomodulin 1 | 0.607 | 2042039-2042410 |
| 6084 | 1083 | bridging integrator 3 | 4.997 | 2047682-2048036 |
| 6099 | 1080 | actin related protein 2/3 complex, subunit 5-like | 28.479 | 2053256-2053599 |
| 6141 | 1070 | aurora kinase B | 6.311 | 2068620-2068994 |
| 6179 | 1062 | CDC42 effector prot (Rho GTPase binding) 4 | 0.921 | 2082109-2082464 |
| 6258 | 1043 | gene trap ROSA b-geo 22 | 28.608 | 2111007-2111388 |
| 6278 | 1038 | tubulin tyrosine ligase-like family, member 5 | 0.196 | 2117720-2118059 |
| 6291 | 1035 | formin binding protein 1 | 0.384 | 2122377-2122769 |
| 6318 | 1030 | centrin 2 | 4.69 | 2131765-2132103 |
| 6338 | 1027 | FERM domain containing 8 | 1.308 | 2139056-2139394 |
| 6354 | 1023 | centrosomal protein 70 | 0.548 | 2144764-2145134 |
| 6460 | 997 | ELMO/CED-12 domain containing 3 | 3.427 | 2182595-2182941 |
| 6467 | 995 | Leber congenital amaurosis 5 (human) | 0.247 | 2185145-2185497 |
| 6500 | 987 | phosphodiesterase 4D, cAMP specific | 0.47 | 2196755-2197145 |
| 6516 | 984 | dystrophin, muscular dystrophy | 0.119 | 2202415-2202812 |
| 6526 | 983 | huntingtin interacting protein 1 related | 0.441 | 2205988-2206301 |
| 6534 | 981 | discs, large (Drosophila) homolog- | 3.759 | 2208846-2209155 |
| | | associated protein 5 | | |
| 6553 | 976 | RIKEN cDNA 2810433K01 gene | 2.289 | 2215581-2215976 |
| 6581 | 971 | HAUS augmin-like complex, subunit 1 | 5.105 | 2225453-2225779 |
| 6599 | 969 | epsilon-tubulin 1 | 0.387 | 2232061-2232442 |
| 6626 | 964 | centrosomal protein 290 | 0.132 | 2241296-2241579 |
| 6651 | 958 | family with sequence similarity 82, member B | 0.84 | 2250023-2250412 |
| 6713 | 943 | centrosomal protein 250 | 0.433 | 2271720-2272085 |
| 6735 | 938 | tropomyosin 3, gamma | 2.656 | 2279590-2279877 |
| 6766 | 934 | family with sequence similarity 82, member A1 | 0.931 | 2290005-2290384 |
| 6782 | 929 | nuclear distribution gene E homolog 1 | 7.884 | 2295836-2296146 |
| | | (A. nidulans) | | |
| 6806 | 924 | purine-nucleoside phosphorylase 1 | 10.99 | 2304356-2304474 |
| 6822 | 920 | tropomyosin 2, beta | 4.355 | 2309610-2309944 |
| 6832 | 917 | RIKEN cDNA 2700060E02 gene | 9.838 | 2313045-2313404 |
| 6834 | 917 | v-abl Abelson MLV oncogene homolog 2 | 0.618 | 2313687-2314064 |
| | | (arg, Abelson-related gene) | | |
| 6882 | 908 | aurora kinase C | 14.218 | 2329723-2330035 |
| 6898 | 903 | spindle assembly 6 homolog (C. elegans) | 0.224 | 2334515-2334801 |
| 6953 | 892 | nucleotide binding protein 2 | 2.113 | 2352711-2353003 |
| 6991 | 884 | dynactin 6 | 9.052 | 2365341-2365718 |
| 7003 | 880 | neural precursor cell expressed, | 0.33 | 2369333-2369684 |
| | | developmentally down-regulated gene 1 | | |
| 7007 | 879 | diacylglycerol kinase, theta | 0.274 | 2370707-2371011 |
| 7040 | 872 | CDC42 small effector 1 | 2.926 | 2382000-2382296 |
| 7096 | 860 | slingshot homolog 2 (Drosophila) | 0.517 | 2400517-2400895 |
| 7125 | 853 | profilin 1 | 11.177 | 2410108-2410492 |
| 7146 | 848 | RIKEN cDNA 2410017P07 gene | 1.326 | 2417114-2417508 |
| 7175 | 841 | baculoviral IAP repeat-containing 5 | 0.966 | 2426437-2426713 |
| 7208 | 836 | leucine rich repeat and coiled-coil domain | 0.248 | 2437517-2437910 |
| | | containing 1 | | |
| 7236 | 829 | DNA segment, Chr 15, Wayne State | 0.268 | 2446945-2447339 |
| | | University 169, expressed | | |
| 7249 | 826 | RIKEN cDNA 4922501C03 gene | 0.438 | 2451461-2451761 |
| 7291 | 813 | HAUS augmin-like complex, subunit 2 | 3.496 | 2464662-2464966 |
| 7323 | 806 | dynein light chain LC8-type 1 | 4.71 | 2475322-2475644 |
| 7330 | 803 | MAD2L1 binding protein | 3.685 | 2477746-2478077 |
| 7365 | 796 | cDNA sequence BC023882 | 0.603 | 2489301-2489640 |
| 7368 | 795 | RIKEN cDNA 6720456B07 gene | 3.581 | 2490278-2490569 |
| 7378 | 793 | tubulin folding cofactor B | 8.324 | 2493603-2493993 |
| 7379 | 793 | ankyrin repeat, family A (RFXANK-like), 2 | 0.45 | 2493994-2494317 |
| 7426 | 782 | engulfment and cell motility 1, ced-12 | 0.528 | 2509515-2509793 |
| | | homolog (C. elegans) | | |
| 7472 | 769 | palladin, cytoskeletal associated protein | 0.53 | 2524218-2524585 |
| 7486 | 767 | melanophilin | 0.258 | 2529148-2529507 |
| 7488 | 766 | WAS protein family, member 2 | 0.188 | 2529775-2530138 |
| 7552 | 749 | mitogen-activated protein kinase 1 | 1.45 | 2550744-2551112 |
| | | interacting protein 1 | | |
| 7588 | 742 | vinculin | 0.23 | 2562670-2562963 |
| 7654 | 724 | dynamin binding protein | 0.201 | 2583481-2583787 |
| 7756 | 700 | Rap1 interacting factor 1 homolog (yeast) | 0.083 | 2618117-2618471 |
| 7794 | 691 | giant axonal neuropathy | 0.587 | 2631132-2631429 |
| 7826 | 683 | Mediterranean fever | 0.311 | 2642002-2642302 |
| 7889 | 664 | ubiquitously expressed transcript | 1.147 | 2662979-2663371 |
| 7899 | 659 | ADP-ribosylation factor-like 3 | 2.999 | 2666529-2666853 |
| 7902 | 658 | intraflagellar transport 20 homolog | 4.021 | 2667596-2667912 |
| | | (Chlamydomonas) | | |
| 7904 | 657 | gamma-aminobutyric acid receptor | 2.814 | 2668257-2668616 |
| | | associated protein | | |
| 7937 | 649 | trichoplein, keratin filament binding | 0.484 | 2679418-2679802 |
| 7975 | 639 | tubulin, beta 3 | 2.093 | 2692217-2692262 |
| 7978 | 639 | BCL2 modifying factor | 0.17 | 2692923-2693205 |
| 8020 | 627 | Rho GTPase-activating protein | 0.115 | 2706942-2707263 |
| 8031 | 622 | B9 protein domain 2 | 12.725 | 2710630-2711005 |
| 8052 | 613 | ARP6 actin-related protein 6 homolog | 0.57 | 2717297-2717635 |
| | | (yeast) | | |
| 8079 | 606 | ADP-ribosylation factor-like 2 | 4.584 | 2726337-2726723 |
| 8108 | 598 | thymosin, beta 4, X chromosome | 24.043 | 2734875-2735269 |
| 8123 | 594 | citron | 0.131 | 2740025-2740319 |
| 8147 | 588 | ankyrin 1, erythroid | 0.072 | 2747574-2747883 |
| 8176 | 583 | dynactin 3 | 1.37 | 2756466-2756744 |
| 8301 | 547 | UBX domain protein 11 | 0.465 | 2797362-2797669 |
| 8335 | 539 | par-3 (partitioning defective 3) homolog | 0.154 | 2808716-2809107 |
| | | (C. elegans) | | |
| 8358 | 532 | myomesin 1 | 0.149 | 2816775-2817100 |
| 8401 | 519 | erythrocyte protein band 4.1-like 5 | 0.187 | 2831535-2831924 |
| 8402 | 519 | ciliary rootlet coiled-coil, rootletin | 0.102 | 2831925-2832268 |
| 8451 | 506 | catenin (cadherin associated protein), alpha 2 | 0.122 | 2848720-2849102 |
| 8516 | 492 | filamin C, gamma | 0.062 | 2869737-2870106 |
| 8610 | 475 | erythrocyte protein band 4.1-like 4a | 0.123 | 2900673-2901022 |
| 8638 | 469 | formin 1 | 0.04 | 2910464-2910758 |
| 8660 | 465 | kinesin family member 2A | 0.256 | 2917368-2917680 |
| 8677 | 463 | pericentriolar material 1 | 0.077 | 2922701-2923049 |
| 8680 | 462 | 4HAUS augmin-like complex, subunit 8 | 1.099 | 2923769-2924049 |
| 8771 | 446 | calcium binding and coiled-coil domain 2 | 1.247 | 2952270-2952623 |
| 8808 | 439 | centrin 3 | 1.05 | 2963461-2963764 |
| 8846 | 430 | thymosin, beta 10 | 5.35 | 2975022-2975322 |
| 8852 | 429 | actin related protein M1 | 0.429 | 2976880-2977149 |
| 8881 | 421 | protein (peptidyl-prolyl cis/trans isomerase) | 1.474 | 2985485-2985777 |
| | | NIMA-interacting, 4 (parvulin) | | |
| 8907 | 416 | formin binding protein 1-like | 0.166 | 2992924-2993272 |
| 8931 | 410 | kinesin family member 1B | 0.059 | 2999526-2999824 |
| 8932 | 409 | SAC3 domain containing 1 | 0.452 | 2999825-3000109 |
| 9057 | 376 | FYVE, RhoGEF and PH domain containing 4 | 0.233 | 3032941-3033212 |
| 9073 | 372 | spectrin alpha 1 | 0.087 | 3037049-3037302 |
| 9085 | 369 | glucocorticoid receptor DNA binding factor 1 | 0.084 | 3040212-3040461 |
| 9137 | 356 | FERM, Rho GEF and pleckstrin domain | 0.091 | 3053199-3053462 |
| | | protein 2 | | |
| 9147 | 353 | tetratricopeptide repeat domain 8 | 0.154 | 3055412-3055707 |
| 9161 | 349 | RAB GTPase activating protein 1 | 0.083 | 3058415-3058689 |
| 9222 | 337 | inversin | 0.261 | 3071675-3071916 |
| 9322 | 307 | Ras and Rab interactor 1 | 0.073 | 3093895-3094135 |
| 9361 | 297 | tubulin cofactor A | 1.057 | 3102113-3102283 |
| 9367 | 295 | Fc receptor, IgG, low affinity IIb | 0.189 | 3103313-3103351 |
| 9473 | 256 | engulfment and cell motility 3, ced-12 | 0.119 | 3122400-3122588 |
| | | homolog (C. elegans) | | |
| 9486 | 251 | ninein | 0.027 | 3124390-3124544 |
| 9587 | 211 | tensin 4 | 0.089 | 3138556-3138633 |
| 9617 | 200 | tropomodulin 2 | 0.02 | 3141512-3141584 |
| 9677 | 173 | radial spoke head 9 homolog | 0.187 | 3146825-3146949 |
| | | (Chlamydomonas) | | |
| 9701 | 161 | Rho family GTPase 1 | 0.152 | 3148560-3148596 |
| 9750 | 135 | actin-binding LIM protein 2 | 0.039 | 3151347-3151451 |
| 9753 | 132 | actin, alpha 1, skeletal muscle | 0.093 | 3151503-3151520 |
| 3157155 | 394 | adducin 2 (beta) | 0.13 | 3183484-3183583 |
| 3157158 | 478 | envoplakin | 0.075 | 3268105-3268204 |
| 3157230 | 310 | LIM domain binding 3 | 0.065 | 3227217-3227316 |
| 3157293 | 264 | formin homology 2 domain containing 3 | 0.259 | 3166084-3166183 |
| 3157294 | 353 | family with sequence similarity 33, member A | 0.31 | 3266905-3267004 |
| 3157319 | 402 | HAUS augmin-like complex, subunit 5 | 0.183 | 3232617-3232716 |
| 3157357 | 735 | PDZ and LIM domain 2 | 1.175 | 3190471-3190570 |
| 3157369 | 1826 | RIKEN cDNA 2310014H01 gene | 2.285 | 3250317-3250416 |
| 3157379 | 252 | P140 gene | 0.045 | 3193771-3193870 |
| 3157397 | 305 | receptor-associated protein of the synapse | 0.188 | 3284249-3284348 |
| 3157398 | 818 | protein Tyr phosphatase, non-receptor type 4 | 0.196 | 3205097-3205196 |
| 3157429 | 809 | sarcoglycan, (dystrophin- | 2.184 | 3188371-3188470 |
| | | associated glycoprotein) | | |
| 3157432 | 395 | myosin VIIA | 0.054 | 3266305-3266404 |
| 3157485 | 2014 | ubiquitin protein ligase E3 component n- | 0.639 | 3209658-3209757 |
| | | recognin 4 | | |
| 3157505 | 644 | crystallin, alpha B | 0.99 | 3280749-3280848 |
| 3157523 | 803 | centromere protein V | 3.696 | 3267205-3267304 |
| 3157536 | 307 | FYVE, RhoGEF & PH domain containing 3 | 0.099 | 3205697-3205796 |
| 3157684 | 329 | doublecortin-like kinase 2 | 0.081 | 3170684-3170783 |
| 3157726 | 995 | desmin | 1.2 | 3159421-3159520 |
| 3157766 | 167 | radial spoke head 4 homolog A | 0.117 | 3201497-3201596 |
| | | (Chlamydomonas) | | |
| 3157768 | 326 | myosin regulatory light chain interacting protein | 0.109 | 3182784-3182883 |
| 3157794 | 2378 | tribbles homolog 2 (Drosophila) | 1.6 | 3204997-3205096 |
| 3157801 | 379 | tropomodulin 4 | 0.308 | 3221891-3221990 |
| 3157821 | 390 | tubulin tyrosine ligase-like family, member 11 | 0.203 | 3159121-3159220 |
| 3157887 | 312 | dynamin 3 | 0.068 | 3263705-3263804 |
| 3157957 | 150 | erythrocyte protein band 4.2 | 0.043 | 3187271-3187370 |
| 3157963 | 1734 | symplekin | 1.095 | 3217191-3217290 |
| 3158016 | 395 | FERM domain containing 5 | 0.095 | 3184871-3184970 |
| 3158035 | 383 | septin 1 | 0.279 | 3259205-3259304 |
| 3158061 | 191 | protein Tyr phosphatase, non-receptor type 3 | 0.03 | 3234617-3234716 |
| 3158125 | 849 | actin related protein 2/3 complex, subunit 3 | 23.491 | 3268805-3268904 |
| 3158127 | 427 | tubulin tyrosine ligase-like family, member 3 | 0.294 | 3262005-3262104 |
| 3158142 | 421 | myosin binding protein C, slow-type | 0.554 | 3202897-3202996 |
| 3158154 | 347 | microtubule-associated protein tau | 0.08 | 3245217-3245316 |
| 3158176 | 705 | IAP promoted placental gene | 0.395 | 3167984-3168083 |
| 3158226 | 185 | tripartite motif-containing 36 | 0.119 | 3176384-3176483 |
| 3158232 | 888 | Wiskott-Aldrich syndrome homolog (human) | 0.603 | 3278849-3278948 |
| 3158242 | 3612 | SMEK homolog 2, suppressor of mek1 | 9.065 | 3210258-3210357 |
| | | (Dictyostelium) | | |
| 3158328 | 1531 | RAB11 family interacting protein 3 (class II) | 0.975 | 3221991-3222090 |
| 3158344 | 550 | cortactin binding protein 2 | 0.187 | 3165384-3165483 |
| 3158346 | 482 | NA | 0.075 | 3269396-3269495 |
| 3158380 | 464 | tubulin Tyr ligase-like family, member 6 | 0.277 | 3185771-3185870 |
| 3158411 | 1049 | erythrocyte protein band 4.9 | 0.588 | 3254017-3254116 |

1. A method for producing an immunogenic agent in a large scale host cell culture, comprising:
(a) contacting a host cell in a large scale host cell culture with at least a first RNA effector molecule, a portion of which is complementary to at least one target gene of a host cell,
(b) maintaining the host cell culture for a time sufficient to modulate expression of the at least one first target gene, wherein the modulation of expression improves production of an immunogenic agent in the host cell;
(c) isolating the immunogenic agent from the host cell;
wherein the large scale host cell culture is at least 1 Liter in size, and wherein the host cell is contacted with at least a first RNA effector molecule by addition of the RNA effector molecule to a culture medium of the large scale host cell culture such that the target gene expression is transiently inhibited.

2. (canceled)

3. The method of claim 1, wherein the host cell in the large scale host cell culture is contacted with a plurality of RNA effector molecules, wherein the plurality of RNA effector molecules modulate expression of at least one target gene, at least two target genes, or a plurality of target genes.

4. A method for production of an immunogenic agent in a cell, the method comprising:
(a) contacting a host cell with a plurality of RNA effector molecules, wherein the two or more RNA effector molecules modulate expression of a plurality of target genes;
(b) maintaining the cell for a time sufficient to modulate expression of the plurality of target genes, wherein the modulation of expression improves production of the immunogenic agent in the cell; and
(c) isolating the immunogenic agent from the cell,
wherein the plurality of target genes comprises at least Bax, Bak, and LDH.

5. The method of claim 4, wherein the host cell is contacted with the plurality of RNA effector molecules by addition of the RNA effector molecule to a culture medium of the large scale host cell culture such that the target gene expression is transiently inhibited.

6. The method of claim 1, wherein the RNA effector molecule, comprises a double-stranded ribonucleic acid (dsRNA), wherein said dsRNA comprises at least two sequences that are complementary to each other and wherein a sense strand comprises a first sequence and an antisense strand comprises a second sequence comprising a region of complementarity which is substantially complementary to at least part of a target gene, and wherein said region of complementarity is 10-30 nucleotides in length.

7. The method of claim 1, wherein the contacting step is performed by continuous infusion of the RNA effector molecule into the culture medium used for maintaining the host cell culture to produce the immunogenic agent.

8. The method of claim 1 wherein the modulation of expression is inhibition of expression, and wherein the inhibition is a partial inhibition.

9. The method of claim 8, wherein the partial inhibition is no greater than a percent inhibition selected from the group consisting of: 10%, 15%, 20%, 25%, 30%, 35%, 40%, 45%, 50%, 55%, 60%, 65%, 70%, 75%, 80%, and 85%.

10. The method of claim 1 wherein the contacting step is repeated at least once.

11. The method of claim 1 wherein the contacting step is repeated multiple times at a frequency selected from the group consisting of: 6 hr, 12 hr, 24 hr, 36 hr, 48 hr, 72 hr, 84 hr, 96 hr, and 108 hr.

12. The method of claim 1 wherein the modulation of expression is inhibition of expression and wherein the contacting step is repeated multiple times, or continuously infused, to maintain an average percent inhibition of at least 50% for the target gene(s) throughout the production of the immunogenic agent.

13. The method of claim 12, wherein the average percent inhibition is selected from the group consisting of at least 50%, at least 60%, at least 70%, at least 80%, at least 90%, at least 95%, at least 99%, or 100%.

14. The method of claim 1 wherein the RNA effector molecule is contacted at a concentration of less than 100 nM.

15. The method of claim 1 wherein the RNA effector molecule is contacted at a concentration of less than 20 nM.

16. The method of claim 1 wherein said contacting a host cell in a large scale host cell culture with a RNA effector molecule is done at least 6 hr, at least 12 hr, at least 18 hr, at least 36 hr, at least 48 hr, at least 60 hr, at least 72 hr, at least 96 hr, or at least 120 hr, or at least 1 week, before isolation of the immunogenic agent or prior to harvesting the supernatant.

17. The method of claim 1 wherein the RNA effector molecule is composition formulated in a lipid formulation.

18. (canceled)

19. The method of claim 1 wherein the RNA effector molecule is not shRNA.

20. The method of claim 1 wherein the RNA effector molecule is siRNA.

21. The method of claim 1 wherein the RNA effector molecule is chemically modified.

22-143. (canceled)
